(19) United States
(12) Patent Application Publication
(10) Pub. No.: US 2009/0274682 A1
(43) Pub. Date: Nov. 5, 2009

(54) DEMETHYLATION AND INACTIVATION OF PROTEIN PHOSPHATASE 2A

(75) Inventors: Yigong Shi, Plainsboro, NJ (US); Yongna Xing, Plainsboro, NJ (US)

Correspondence Address: PEPPER HAMILTON LLP, ONE MELLON CENTER, 50TH FLOOR, 500 GRANT STREET, PITTSBURGH, PA, 15219, US

(73) Assignee: THE TRUSTEES OF PRINCETON UNIVERSITY, Princeton, NJ (US)

(21) Appl. No.: 12/365,532

(22) Filed: Feb. 4, 2009

Related U.S. Application Data
(60) Provisional application No. 61/063,636, filed on Feb. 5, 2008.

(51) Int. Cl. A61K 38/47; C12N 9/16; C12Q 1/42; G01N 33/573; G06F 19/00

(52) U.S. Cl. 424/94.61; 435/196; 435/21; 435/7.8; 702/27

(57) ABSTRACT

Embodiments of the present invention relate to atomic coordinates for PME-1 alone or in complex with PP2A, as well as methods for using these atomic coordinates to prepare inhibitors of PME-1 and/or PP2A and inhibitors prepared using such methods. Further embodiments relate to biochemical analyses of the interactions of PME-1 alone or in complex with PP2A. Further embodiments relate to compositions including mimetics and small molecules, optionally, secondary agents, which may be used to treat disorders in which PME-1 and/or PP2A activity plays a contributing role.

### CROSS-REFERENCE TO RELATED APPLICATIONS

This application claims priority to and benefit of U.S. Provisional Application No. 61/063,636 entitled “Structural Mechanism of Demethylation and Inactivation of Protein Phosphatase 2A”, filed on Feb. 5, 2008; the entire contents of which is hereby incorporated by reference in its entirety.

### GOVERNMENT INTERESTS

This invention was made with U.S. Government support (NIH Grant No. R01 CA123155) and the U.S. Government may therefore have certain rights in the invention.

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

1. Field of Invention

Not applicable

2. Description of Related Art

Not applicable

### BRIEF SUMMARY OF THE INVENTION

In some embodiments the present invention provides compositions comprising a crystal of PME-1. In some other embodiments, the present invention provides a composition comprising a crystal comprising PME-1 and PP2A.

In some embodiments, the present invention provides methods for preparing a PME-1 modulating compound comprising applying a three-dimensional molecular modeling algorithm to the atomic coordinates of at least a portion of PME-1 alone or in complex with PP2A; determining spatial coordinates of the at least a portion of PME-1 alone or in complex with PP2A; electronically screening stored spatial coordinates of candidate compounds against the spatial coordinates of the at least a portion of PME-1 alone or in complex with PP2A; identifying a compound that is substantially similar to the at least a portion of PME-1 alone or in complex with PP2A; and synthesizing the identified compound.

In some other embodiments, the present invention provides pharmaceutical compositions comprising an effective amount of a compound having a three-dimensional structure corresponding to atomic coordinates of at least a portion of PME-1 and a pharmaceutically acceptable excipient or carrier.

In some embodiments, the present invention provides for systems for identifying PME-1 and/or PP2A modulators comprising a processor; and a processor readable storage medium in communication with the processor readable storage medium comprising the atomic coordinates of at least a portion of PME-1 alone or in complex with PP2A.

In some embodiments, the present invention provides for PME-1 and/or PP2A binding compounds comprising a molecule having a three-dimensional structure corresponding to atomic coordinates derived from at least a portion of an atomic model of PME-1 alone or in complex with PP2A.

### DESCRIPTION OF DRAWINGS

For a fuller understanding of the nature and advantages of the present invention, reference should be made to the following detailed description taken in connection with the accompanying drawings. The file of this patent contains at least one drawing/photograph executed in color. Copies of this patent with color drawing(s)/photograph(s) will be provided to the USPTO upon request and payment of the necessary fee. All figures where structural representations are shown were prepared using MOLSCRIPT (Kraulis (1991) J Appl Crystallogr 24:946-950) and GRASP (Nicholls et al. (1991) Proteins: Struct Funct Genet. 11:281-296).

FIG. 1. Structure of the PP2A-specific methyl esterase-1 (PME-1). (A) Structure of PME-1 in stereo. The core structural elements are colored cyan and the cap domain in orange. The catalytic residue Ser156 is labeled. (B) Sequence alignment of PME-1 from human, frog, fruit fly, worm, and yeast. Secondary structural elements are indicated above the alignment.

FIG. 2. Structure of PME-1 bound to the PP2A core enzyme. (A) Structure of the PP2A-PME-1 complex in two views related by a vertical rotation of 90 degrees. The scaffold and catalytic subunits are colored green and blue, respectively. PME-1 is shown in yellow. The carboxy-terminal peptide is highlighted in magenta. The coloring scheme is preserved in FIGS. 3-5. (B) Structure overlay of the PP2A-PME-1 complex and the PP2A core enzyme. The A and C subunit of the PP2A core enzyme are colored pink and cyan, respectively. This analysis indicates that PME-1 does not directly bind to the A subunit. (C) A slice of the surface representation of PME-1 to show the recognition of the carboxy-terminal peptide of the C subunit. Leu309 is labeled.

FIG. 3. Interface between PME-1 and the C subunit of the PP2A core enzyme. (A) PME-1 and the C subunit form an S-shaped interface. A slice of the complex is shown. (B) A close-up view of the interface in stereo. Residues from PME-1 and the C subunit are shown in green and magenta, respectively. H-bonds are represented by dotted lines.

FIG. 4. Activation of PME-1. (A) Recognition of the carboxy-terminal peptide of the C subunit by PME-1. In this stereo view, 2Fo-Fc electron density is shown at 1.5σ level around the peptide (magenta). H-bonds are represented by dotted lines. (B) Structural comparison of free PME-1 (cyan) and the PP2A-bound PME-1 (yellow). Only regions around the active site of PME-1 are shown. The carboxy-terminal peptide of the C subunit is shown in magenta. (C) Stereo view of the conformational changes at the active site. d, Mutation of the catalytic triad residues resulted in marked reduction of the methylesterase activity of PME-1.

FIG. 5. Inactivation of PP2A by PME-1. (A) A stereo view of the PME-1-PP2A interface at the active site region of the C subunit. 2Fo-Fc electron density is shown at 1.5σ level surrounding side chains of Met335 in PME-1 and the residues that are involved in binding to metal atoms in the C subunit. (B) Structural overlay of the PME-1-PP2A interface with the active site of the C subunit in the PP2A core enzyme. The C subunit in the PP2A core enzyme is colored cyan, with the side chains shown in gold. The two manganese metal atoms and okadaic acid are colored grey.

FIG. 6. A structure-based model of PME-1 function in PP2A regulation. In this model, PME-1 has two roles: demethylation and inactivation of PP2A.

FIG. 7. Deletion of the HEAT repeats 2-10 in the scaffold subunit has no impact on the methylesterase activity of PME-1. In this assay, the PP2A core enzyme (AC heterodimer) was methylated by LCMT1 and used as a substrate for the methylesterase activity of PME-1. The esterase activity of PME-1 on the AC dimer involving the truncated A subunit (AΔN) was nearly identical to that on the AC dimer involving the full-length A subunit. This result suggests that deletion of HEAT repeats 2-10 in the A subunit has no detectable impact on the interaction between PME-1 and the PP2A core enzyme.

FIG. 8. Mutational analysis of PME-1 residues at the interface between PME-1 and PP2A. The impact of the mutations on the interaction between PME-1 and PP2A was evaluated by the methylesterase activity of the corresponding PME-1 mutants using methylated PP2A as a substrate. Since these mutations did not involve the residues at the active site of PME-1, a decrease in the methylesterase activity likely reflects a weakened binding affinity between PP2A and the specific PME-1 mutant. The side chain of Arg369 participates in multiple inter-molecular hydrogen bonds with PP2A. Its mutation to Asp is expected to reduce the binding affinity between PME-1 and PP2A. In contrast, the side chains of Gln334 and Gln336 in PME-1 are not involved in any interaction with PP2A; their mutations are predicted not to affect the PME-1-PP2A interaction. The experimental observations confirmed these predictions.

FIG. 9. PMSF only inactivated PME-1 that was pre-incubated with a PP2A core enzyme involving truncation of the carboxy-terminus of the catalytic subunit. PMSF does not inactivate PME-1 alone, because the active site of PME-1 is not in a productive conformation. When PME-1 is bound to the PP2A core enzyme, the active site of PME-1 is rearranged into a productive conformation, which is predicted to make the catalytic serine residue susceptible to PMSF inactivation. To confirm this structure-based prediction, we pre-incubated PME-1 with a variant of the PP2A core enzyme involving truncation of the carboxy-terminus of the catalytic subunit and then added freshly prepared PMSF. The catalytic subunit of the PP2A variant was digested by chymotrypsin to remove two amino acids at the carboxy-terminus. This truncated catalytic subunit was used so that PMSF could gain access to the active site of PME-1. The treated PME-1 was assayed for its methylesterase activity and compared with the untreated PME-1. This result shows that pre-incubation with the PP2A variant and PMSF led to marked reduction of PME-1-mediated methylesterase activity.

FIG. 10. PME-1-mediated metal removal and inactivation of PP2A. The PP2A-PME-1 complex was incubated on ice overnight in the presence of Mn²⁺ followed by fractionation using size exclusion chromatography in the absence of metal ions. (A) The levels of Mn²⁺ bound to PP2A or PP2A-PME-1 complex were quantified by ICP-MS and normalized to the metal level in PP2A alone. (B) The phosphatase activity of PP2A or the PP2A-PME-1 complex was measured using ³²P-labeled phosphorylase a in the presence or absence of manganese chloride (50 μM).

FIG. 11. PME-1-mediated inactivation of PP2A. (A) Phosphatase activity of PP2A in the presence of various concentrations of PME-1. The phosphatase activity was measured upon initial incubation of PP2A and PME-1. Phosphorylase a was used as a substrate. (B) Phosphatase activity of PP2A and the PP2A-PME-1 complex after incubation at 37° C. for the indicated duration. For comparison, EDTA treatment of PP2A also markedly reduced the phosphatase activity of PP2A, presumably due to chelation of the catalytic metal ions.

### DETAILED DESCRIPTION

It must be noted that, as used herein and in the appended claims, the singular forms “a,” “an,” and “the” include plural reference unless the context clearly dictates otherwise. Unless defined otherwise, all technical and scientific terms used herein, have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

The terms “mimetic,” “peptide mimetic,” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically produced peptides, recombinantly or chemically modified peptides, as well as non-peptide agents, such as small molecule drug mimetics as further described below. Mimetic compounds can have additional characteristics that enhance their therapeutic application, such as increased cell permeability, greater affinity and/or avidity, and prolonged biological half-life.

As used herein, the terms “pharmaceutically acceptable,” “physiologically tolerable,” and grammatical variations thereof, as they refer to compositions, carriers, diluents, and reagents, are used interchangeably and represent that the materials are capable of administration upon a mammal without the production of undesirable physiological effects such as nausea, dizziness, rash, or gastric upset.

“Providing,” when used in conjunction with a therapeutic, means to administer a therapeutic directly into or onto a target tissue, or to administer a therapeutic to a patient whereby the therapeutic positively impacts the tissue to which it is targeted.

As used herein, “subject,” “patient” or “individual” refers to an animal or mammal including, but not limited to, a human, dog, cat, horse, cow, pig, sheep, goat, chicken, monkey, rabbit, rat, or mouse, etc.

As used herein, “coordinates” or “atomic coordinates” refers to the coordinates of a molecular structure, such as a protein structure. The protein structure can be, for example, a complex. In some embodiments, the coordinates referred to herein can be, for example, the coordinates discloses in Appendix A and/or Appendix B.

As used herein, the term “therapeutic” means an agent utilized to treat, combat, ameliorate, prevent or improve an unwanted condition or disease of a patient. Embodiments of the present invention are directed to promote apoptosis and thus, cell death.

The terms “therapeutically effective amount” or “effective amount,” as used herein, may be used interchangeably and refer to an amount of a therapeutic compound component of the present invention. For example, a therapeutically effective amount of a therapeutic compound is a predetermined amount calculated to achieve the desired effect, i.e., to effectively modulate the activity of PME-1 and/or protein phosphatase 2A (PP2A).

“Inhibitor” means a compound which reduces or prevents a particular interaction or reaction. For example, an inhibitor may bind to PP2A C-subunit inactivating the C-subunit and inhibiting the phosphotyrosyl activity of PP2A. An inhibitor may also inhibit the interaction between PME-1 and PP2A. An inhibitor may also inhibit the enzymatic activity of PME-1.

“Pharmaceutically acceptable salts” include both acid and base addition salts. “Pharmaceutically acceptable acid addition salt” refers to those salts which retain the biological effectiveness and properties of the free bases and which are not biologically or otherwise undesirable and formed with inorganic acids, such as hydrochloric acid, hydrobromic acid, sulfuric acid, nitric acid, carbonic acid, phosphoric acid, and the like. Organic acids may be selected from aliphatic, cycloaliphatic, aromatic, araliphatic, heterocyclic, carboxylic, and sulfonic classes of organic acids, such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid, and the like.

Protein phosphatase 2A (PP2A) is a major serine/threonine phosphatase with complex compositions, and is involved in many essential aspects of cellular function. Deregulation of PP2A has been linked to many debilitating diseases such as cancer and Alzheimer's disease. The heterodimeric PP2A core enzyme consists of a 36-kD catalytic subunit, or C subunit, and a 65-kD scaffold subunit, or A subunit. To gain full activity toward specific substrates, the PP2A core enzyme associates with a variable regulatory subunit to form a heterotrimeric holoenzyme. The regulatory subunits are divided into four structurally distinct families, B (B55 or PR55), B′ (B56 or PR61), B″ (PR72), and B′″ (PR93/PR110).

Reversible methylation of the PP2A core enzyme is a conserved and essential regulatory mechanism. Methylation of the carboxy-terminal Leu309 in a conserved TPDYFL309 motif of the C subunit has been shown to enhance the affinity of the PP2A core enzyme for some, but not all, regulatory subunits. Intriguingly, changes in this peptide motif also affected interaction of the C subunit with the α4 protein, presumably through alteration of methylation, and led to a complex with distinct substrate specificity that is essential for cell survival. Thus changes in PP2A methylation appear to regulate formation of PP2A complexes and consequently shift the specificity of PP2A phosphatase activity in cells. Supporting this notion, blockade of PP2A methylation in yeast caused a set of phenotypes that are consistent with decreased formation of PP2A holoenzymes.

Reversible methylation of PP2A is catalyzed by two highly conserved enzymes, a 38-kD PP2A-specific leucine carboxyl methyltransferase (LCMT1) and a 42-kD PP2A-specific methylesterase (PME-1). PME-1 catalyzes removal of the methyl group, thus reversing the activity of LCMT1. Over-expression of PP2A methylesterase caused phenotypes similar to those associated with loss of the methyltransferase gene. It had also been demonstrated that methylation levels of PP2A changed during cell cycle, suggesting a critical role of methylation in cell cycle regulation.

Recent evidence hints at a broader role for PME-1 than just hydrolysis of a methylester bond. PME-1 was found to be associated with two inactive mutants of PP2A. A portion of cellular PP2A, which remained stably bound to PME-1, was found to be inactive; interestingly, this inactive portion of PP2A could be re-activated by PP2A phosphatase activator (PTPA), but not by LCMT1, ruling out the possibility that inactivation was solely caused by demethylation. These observations suggest that PME-1 might somehow sequester an inactive portion of PP2A. Surprisingly, however, the phosphatase activity of the re-activated PP2A was no longer subject to inhibition by PME-1. At present, the underlying molecular mechanisms of the interplay between PME-1 and PP2A remain largely unknown and there is a long-felt need for a better understanding regarding this interplay and the identification of modulators of this interaction and the enzymatic activity of PME-1 and/or PP2A.

Embodiments of the present invention fulfills these needs and others by better understanding the regulation of PP2A through the elucidation of the crystal structures of PME-1 by itself and in complex with the PP2A core enzyme. It has now been demonstrated that binding of PME-1 to PP2A results in two opposing consequences: activation of PME-1 through structural rearrangement and inactivation of PP2A through removal of catalytic metal atoms. The dual role of PME-1 likely couples methylation of PP2A with its activation and holoenzyme assembly.

In some embodiments, the polypeptide sequence of PME-1 is SEQ ID NO: 1. The sequence for PME-1 can also be found at Genbank accession number AAD44976 In some embodiments, the polypeptide sequence of the catalytic subunit of PP2A is SEQ ID NO. 2. The sequence for the catalytic subunit can also be found at Genbank accession number NP_{—}002706. In some embodiments, the polypeptide sequence of the A-subunit of PP2A is A-alpha. In some embodiments, the polypeptide sequence of the A-subunit is SEQ ID NO: 3. The sequence for the A-subunit can also be found at Genbank Accession number NP_{—}055040. The sequence of the B-subunit of PP2A can be SEQ ID NO: 4. The sequence of the B-subunit can also be found at GenBank accession number AAC37603. The B-subunit can also be referred to as B56-gamma1.

In some embodiments, the present invention is directed to the atomic coordinates defining PME1 alone and in complex with PP2A. Embodiments of the present invention are also directed to methods for using the atomic coordinates of PME-1 alone or in complex with PP2A, mimetics and small molecules prepared using such methods, and pharmaceutical compositions made from mimetics and small molecules so prepared.

As used throughout, the phrase “PME-1 alone or in complex with PP2A” refers to either PME-1 protein that is free of PP2A subunit or a PME-1 protein molecule that is associated with complex with at least one PP2A subunit. PME-1 that is associated with a PP2A subunit can be associated with the A-subunit of PP2A, B-subunit of PP2A, C-subunit of PP2A, or any combination thereof.

In some embodiments, the present invention is directed to a composition comprising a crystal of PME-1. In some embodiments, the crystal comprises the PME-1 core. As used herein, “PME-1 core” refers to the structural core of PME-1. The structure core of PME-1 may be generated by incubation of full-length PME-1 with a protease, such as, for example, trypsin. The protein can be treated with an effective amount of trypsin (e.g. 0.5 mg/ml) for 20 minutes on ice. After treating with a protease the structural core can be identified.

In certain embodiments, the structural or enzymatic core of PME-1 comprises residues 39-248 of SEQ ID NO: 1.

In further embodiments, the enzymatic or structural core comprises residues 273-386 of SEQ ID NO: 1. IN some embodiments the structural or enzymatic core of PME-1 comprises an N-terminal 38 residue truncation and/or an internal deletion of residues 239-283.

In further embodiments, the structural core comprises residues 39-248 and 273-386 of SEQ ID NO: 1. In further embodiments, residues 39-248 and 273-386 may be linked in a single polypeptide chain. In some embodiments, the linker is a peptide that is 1, 2, 3, 4, 5, amino acid residues. In some embodiments the linker is less than 5 residues, for example, 4 residues, 3 residues, 2 residues or 1 residue. The linker can be, for example, a polypeptide comprising 3 residues wherein the polypeptide has a sequence of EGK.

In various embodiments, the claimed invention relates to methods of preparing crystalline forms of PME-1 alone or in complex with PP2A by providing an aqueous solution comprising PME-1 alone or in complex with PP2A. A reservoir solution comprising a precipitant may be mixed with a volume of the PME-1 alone or in complex with PP2A solution and the resultant mixed volume is crystallized. In some embodiments, the crystals may be dissolved and recrystallized. The crystals can be dissolved with the precipitant in a small amount to minimize dilution effects of the other reagents and left to regrow for a period of time.

The proteins can be prepared by any method to isolate purified proteins, such as isolation from E. Coli that overexpress the proteins of interest. The proteins can then be purified to, for example, homogeneity, by gel filtration chromatography.

In various embodiments of the method of preparing crystalline forms of PME-1 alone or in complexes with PPSA, the concentration of the proteins the aqueous solution may vary, but can be, for example, about 1 to about 50 mg/ml, about 5 to about 15 mg/ml, or about 6 mg/ml. In some embodiments, the concentration of the proteins is about 6 mg/ml or about 15 mg/ml. Similarly, precipitants used in the invention may vary, and may be selected from any precipitant known in the art. Any concentration of precipitant may be used in the reservoir solution. For example, the concentration can be about 20 to 30%. In some embodiments, the concentration is about 24-26% Jeffamine-2001 (v/v) or about 23-25% PEG3350 (v/v). The solutions can also comprise ammonium citrate, for example, in concentrations of about 100 mM.

One skilled in the art will understand that each of these parameters can be varied without undue experimentation and acceptable crystals will still be obtained. In practice, once the appropriate precipitating agents, buffers, or other experimental variables are determined for any given growth method, any of these methods or any other methods can be used to grow the claimed crystals. One skilled in the art can determine the variables depending upon one's particular needs. Various methods of crystallization can be used in the claimed invention, including, for example, vapor diffusion, batch, liquid-bridge, or dialysis crystallization. See, e.g. McPherson et al., Preparation and Analysis of Protein Crystals, Glick, ed. (John Wiley & Co., 1982), pp. 82-159; Jancarik et al., J. Appl. Crystallogr., 24: 409-411 (1991).

In vapor diffusion crystallization, a small volume (i.e., a few milliliters) of protein solution is mixed with a solution containing a precipitant. This mixed volume is suspended over a well containing a small amount, i.e. about 1 ml, of precipitant. Vapor diffusion from the drop to the well will result in crystal formation in the drop.

The dialysis method of crystallization utilizes a semipermeable size-exclusion membrane that retains the protein but allows small molecules (i.e. buffers and precipitants) to diffuse in and out. In dialysis, rather than concentrating the protein and the precipitant by evaporation, the precipitant is allowed to slowly diffuse through the membrane and reduce the solubility of the protein while keeping the protein concentration fixed.

The batch methods generally involve the slow addition of a precipitant to an aqueous solution of protein until the solution just becomes turbid; at this point the container can be sealed and left undisturbed for a period of time until crystallization occurs.

The crystal structure was determined by combined anomalous scattering from intrinsic sulfur and fortuitous bromide ion as discussed in detail in the Example below. An example of a method to prepare crystals of PME-1 include, but is not limited to, hanging-drop vapor-diffusion method. In the hanging-drop vapor-diffusion method the protein may be mixed with an about equal volume of reservoir solution. The reservoir solution can, for example, comprise Jeffamine-2001, sodium chloride, and/or DTT. In some embodiments, the reservoir solution comprises 24-26% Jeffamine 2001 (v/v). In some embodiments, the reservoir solution comprises about 150-250 mM, about 175-225 mM, about 180-220 mM, about 190-210 mM, about 195-205 mM, or about 200 mM sodium chloride. The reservation solution can also comprise about 1-10 mM, about 2-9 mM, about 3-8 mM, about 4-7 mM, about 4-6 mM, about 5-10 mM, about 4.5 to 5.5 mM, or about 5 mM DTT. The reservoir solution can also comprise PEG3350. In some embodiments, the concentration of PEG3350 is about 23-25% (v/v). In some embodiments the reservoir solution comprises ammonium citrate. In some embodiments, the ammonium citrate is present at a concentration of about 50 to about 150 mM, about 75 to about 125 mM, or about 100 mM. In some embodiments, the ammonium citrate is present in amount of about 100 mM. In some embodiments, the method comprises allowing crystals to grow for about 1 week.

Once formed the crystals can be equilibrated in a cryoprotectant buffer containing the reservoir buffer. In some embodiments, the equilibration buffer comprises about 34% (v/v) Jeffamine-2001 or about 34% (v/v) PEG3350, about 20% glycerol (v/v), about 50 μl MnCl₂ or combinations thereof. In some embodiments, the crystals are dehydrated following equilibration. In some embodiments, the crystals are flash frozen after equilibration. The method can also comprise any variation as described in the Examples described herein.

In some embodiments, the crystal of PME-1 has space group P3₁21. In some embodiments, the crystal has unit cell dimensions, ±2%, of a=b=82.5 Å, c=90.8 Å, α=β=90°, γ=120°. The crystal can also comprise one protein molecule in each asymmetric unit.

Further embodiments of the present invention provide crystals comprising PME-1 alone or in complex with PP2A that can diffract for X-ray determination. The crystal can, for example, diffract X-rays for a determination of structure coordinates to a resolution of a value equal to or less than about 5.0, equal to or less than about 4.0, equal to or less than about 3.0, equal to or less than about 2.5 angstroms. The crystals can also, for example, diffract X-rays for a determination of structure coordinates to a resolution of a value equal to about 2.1 or about 2.8 angstroms. The crystals can also, for example, diffract X-rays for a determination of structure coordinates to a resolution of a value equal 2.1 or 2.8 angstroms.

Some embodiments of the present invention can also provide, in some embodiments, a crystal that has the structure that is defined by the coordinates disclosed in Appendix A and/or Appendix B.

In certain embodiments, the crystals comprising a protein, for example, PME-1 alone or in complex with PP2A, can comprise a methionine that is replaced with a selenomethionine.

Embodiments of the present invention provide a composition comprising a crystal of PME-1 complexed with PP2A. In some embodiments, the PP2A comprises the A subunit and the C-subunit of PP2A. The formation of a PP2A complex comprising an A subunit and a C subunit can be formed under conditions that are effective to form the complex. The PP2A A subunit can be a mutant A subunit, such as, for example, a deletion mutant. An example of a deletion mutant of PP2A A subunit is AΔN. In AΔN various HEAT repeats are deleted. In the AΔN the protein comprises HEAT repeats 1 and 11-15 and lacks repeats 2-10. The PP2A protein, for example the core enzyme involving the full-length C subunit (e.g. C_{α}) and PP2A A subunit (e.g. AΔN) can be assembled, for example, as the methods described in Xing et al., 2006, which is hereby incorporated by reference in its entirety.

In some embodiments, to form the complex of PME-1 and PP2A the proteins can be contacted with one another under conditions effective to form a complex. An example of conditions that are effective to form the complex include, but is not limited to, where PP2A is methylated. PP2A can be methylated by any enzyme including, but not limited to, PP2A-specific leucine carboxyl methyltransferase (LCMT1). LCMT1 and PP2A can be incubated in the presence of S=adenosyl methionine (SAM) to facilitate methylation. After methylation PP2A can be contacted with an excess amount of PME-1. In some embodiments, the PME-1 incubated with the methylated PP2A comprising an inactivating mutation that inactivates PME-1's catalytic activity. The mutation can be any mutation that inactivates the methylesterase activity of PME-1. An example of a mutation that inactivates the methylesterase activity of PME-1 includes, but is not limited to, where Serine 156 of PME-1 is mutated. In some embodiments, Serine 156 is mutated to an alanine.

In some embodiments, present invention also provides, in some embodiments, for a crystal comprising a complex of PP2A and PME-1 wherein the crystal has a space group C2. The crystal can, for example, have unit cell dimensions, ±2%, of α=129.3 Å, b=54.8 Å, c=125.2 Å, α=90°, β=111°, γ=90°. The unit cell can comprise, for example, one complex per asymmetric unit. The present invention can also provide in some embodiments a crystal that comprises a complex of PME-1 and PP2A with a structure that is defined by the coordinates of Appendix B.

In some embodiments of the present invention, the compositions can also comprise a crystal of PME-1 alone or in complex with PP2A comprising the properties described in Table 1. In some embodiments, the crystal comprising a complex of PME-1 and PP2A comprises a complex wherein PME-1 binds (i.e. has contact with) the C-subunit of PP2A.

In some embodiments of the present invention, the crystals can be used to generate diffraction data to determine the atomic coordinates of PME-1 alone or in complex with PP2A. The coordinates can be determined using any known method and the coordinates can be used, for example, to construct an atomic model of PME-1 alone or in complex with PP2A. For example, atomic coordinates of PME-1 alone or in complex with PP2A may be determined from crystallographic diffraction data collected using a combination of molecular replacement and single-wavelength anomalous dispersion. The diffraction and structural data described herein include atomic models for PME-1 alone or in complex with PP2A. The atomic model of the complex of PME-1 and PP2A can include, for example, a PP2A complex that comprises an A-subunit and/or a C subunit. The A-subunit can be the AΔN subunit and the C subunit can be the C, subunit.

Various embodiments of the invention are directed to the atomic coordinates of PME-1 alone or in complex with PP2A and the use of these atomic coordinates to design or identify molecules that specifically inhibit or activate PME-1, inhibit or activate PP2A, or inhibit or enhance the binding (e.g. formation of complex) between PME-1 and PP2A. For example, in one embodiment, the atomic coordinates of PME-1 alone or in complex with PP2A may be used to design and/or screen inhibitor molecules that bind to PME-1 and/or PP2A and disrupt or inhibit the binding of PME-1 to PP2A. In another embodiments, the atomic coordinates of PME-1 alone or in complex with PP2A may be used to design and/or screen inhibitor molecules that bind to PME-1 and/or PP2A C subunit and, for example, inhibit the ability of the C-subunit to bind with PME-1. In further embodiments, the atomic coordinates of PME-1 alone or in complex with PP2A may be used to design and/or screen molecules that inhibit the flexibility of PME-1, PP2A subunit A, and/or PP2A subunit C such that PME-1, PP2A subunit A, and or PP2A subunit C may not contact each other or a substrate protein cannot be brought into contact with the active site of PME-1 and/or the C-subunit of PP2A. In still other embodiments, the atomic coordinates of PME-1 alone or in complex with PP2A may be used to design and/or screen activators of PME-1 and/or PP2A by, for example, increasing the affinity of the C-subunit for its substrate or increasing the affinity of PME-1 for its substrate (e.g. PP2A). The atomic coordinates can be those as shown in Appendix A and/or Appendix B. The coordinates can also be found in PDB No. 3C5V and/or 3C5W, each of which are hereby incorporated by reference in their entirety.

Further embodiments comprise methods of designing and/or screening of molecules that inhibit PME-1 and/or PP2A activity. Such methods may include inhibiting the activity of PME-1 and/or PP2A C-subunit and/or inhibiting the ability of the PP2A C-subunit to bind to other components of PP2A core or PP2A holoenzyme. For example, in various embodiments, binding of an inhibitor molecule to PME-1 may selectively reduce or eliminate the activity of PME-1 by reducing the ability of PME-1 to bind to its substrate by, for example, interrupting the binding interface between PME-1 and its substrate. For example, the molecule may inhibit the interactions between PME-1 and the C-subunit of PP2A. In other embodiments, binding of an inhibitor molecule to PME-1 may reduce or eliminate modifications to the C-subunit, such as, for example, methylation by inhibiting binding or activity of activating methyl transferases. In additional embodiments, the atomic coordinates of PME-1 alone or in complex with PP2A described herein may be used to design and/or screen molecules that activate PP2A catalytic activity by, for example, modulating the methylation status of PP2A.

Such molecules as those described herein that for example, inhibit or enhance the binding of PME-1 and PP2A may be designed or screened using any method known in the art. For example, in certain embodiments, the atomic coordinates of PME-1 alone or in complex with PP2A may be identified, reconstituted and/or isolated in silico (i.e., using a computer processor, software, and a computer/user interface) and used to design or screen molecules that may fit within the interface wherein PME-1 binds to PP2A.

Compounds designed or identified using such methods may substantially mimic the shape, size, and/or charge of a portion of PME-1 or the interface of the C-subunit to which PME-1 binds to. For example, the molecule can mimic the structure formed by the carboxy-terminal six amino acids of the C-subunit. These residues can be, for example T₃₀₄PDYFL₃₀₉ (SEQ ID NO: 5) of the C-subunit of PP2A. As described herein, PME-1 binds directly to the active site of the C subunit of PP2A. This interaction is mediated by, for example, helix α9 and/or helix α10 of PME-1. Therefore, a molecule, can be designed to mimic the structure or a portion thereof of helix α9 and/or helix α10 of PME-1 to inhibit the interaction of PME-1 and PP2A.

For example, in one embodiment, a portion of the C-subunit encompassing the atomic coordinates of amino acids 59, 202, 212, 213, 214, 241, 243, 242, 260, or combinations thereof of the C subunit of PP2A (SEQ ID NO: 3) may be used to design and/or screen compounds that substantially mimic the structural features of portions of subunit C of PP2A. The coordinates can be, for example, those described in Appendix B. In some embodiments, a portion of PME-1 encompassing the atomic coordinates of amino acids 332, 334, 335, 336, 340, 369, and 370 may be used to design and/or screen compounds that substantially mimic the structural features of portions of PME-1 and are substantially complementary to the portions that mediate the interaction of PME-1 to the C-subunit of PP2A. Such compounds may bind to PME-1 and/or the C-subunit of PP2A and, for example, inhibit binding of PME-1 to the C-subunit of PP2A or interrupt interactions between the C-subunit and PME-1 thereby inhibiting the methylesterase activity of PME-1 as it is relates to the methylation status of PP2A. In other embodiments, portions of any of the interfaces described and illustrated in any of the figures or coordinates described herein may be used to design and/or screen compounds that may substantially mimic the shape, size, and/or charge of a portion of PME-1 and/or PP2A, including but the portion of PP2A A-subunit and C-subunit, which includes, for example, the interface between PME-1 and PP2A C-subunit.

In some embodiments, a portion of the atomic coordinates defining the C-subunit of PP2A encompassing a binding interface to PME-1 and/or A-subunit may be utilized to design and/or screen compounds that may inhibit PP2A activity or inhibit the interaction between PME-1 and the C-subunit of PP2A. For example, a portion of the atomic coordinates of the C-subunit encompassing any of the interfaces described and illustrated in the figures and coordinates described herein may be reconstituted and/or isolated in silico and used to identify compounds that substantially mimic a portion of the C-subunit and/or are substantially complementary to a portion of PME-1 at the interface between PME-1 and the C-subunit. Compounds identified in such embodiments may bind to PME-1 and inhibit binding of the C-subunit or interrupt interactions at the interface between the PME-1 and C-subunits thereby inhibiting PME-1 activity as it relates to the methylation status of PP2A.

Other embodiments of the invention include molecules designed and screened to bind to PME-1 and inhibit various aspects of PME-1 activity. For example, in one embodiment, an inhibitor may be designed or molecules may be screened and identified that binds to PME-1 in a similar manner to the C-subunit. For example, a molecule may be identified that binds to a portion of PME-1 encompassing the residues that can bind to the C-subunit. For example, residues 85, 157, 181, 194, 197, 198, 291, 298, 301, 325, 349 for a pocket that can encompass the side chains of residues 307 and 309 of the C subunit of PP2A. Therefore, a molecule can be identified that mimics these structural features or others described herein as it relates to the interaction between PME-1 and PP2A.

In still other embodiments, an inhibitor may be designed or a molecule may screened and identified that inhibits or reduces the flexibility of the C-subunit thereby, for example, reducing or eliminating the ability of the C-subunit to bind or interact with PME-1, thereby modulating the methylation status of PP2A. Embodiments including the design or screening of inhibitors which reduce flexibility of the C-subunit may include designing or screening any number of compounds which interact with the C-subunit in any number of ways.

In particular embodiments, the inhibitors may be identified or designed that bind to the groove of PME-1 that interacts with the C-subunit. This groove is defined by residues 194, 197, 198, 291, 298 and 301 of PME-1. In some embodiments the groove is defined by the coordinates of those residues as described in Appendix B. Such inhibitors may bind to the groove and inhibit the interaction of PME-1 to the C-subunit. For example, such an inhibitor may include a shape that is at least partially complementary to a portion of the concave side of the B-subunit. Such an inhibitor may also include one or more structural features associated with any number of substrate proteins. For example, substrate proteins may be aligned and structural features of portions of the substrate proteins having similarity, may be included as structural features in an inhibitor.

In any of the embodiments described above, a designed or identified inhibitor molecule may have a three-dimensional structure corresponding to at least a portion of PME-1 alone or in complex with PP2A. For example, an inhibitor may be identified by applying a three-dimensional modeling algorithm to the at least a portion of the atomic coordinates of PME-1 alone or in complex with PP2A encompassing, for example, a region of the C-subunit where the inhibitor binds or a region of one or more subunits involved in an interface with PME-1 and electronically screening stored spatial coordinates of candidate compounds against the atomic coordinates of PME-1 alone or in complex with PP2A or a portion thereof. Candidate compounds that are identified as substantially complementary to the portion of the PME-1 molecule alone or in complex with PP2A modeled, or designed to be substantially complementary to the portion of the PME-1 molecule alone or in complex with the PP2A molecule modeled. Candidate compounds so identified may be synthesized using known techniques and then tested for the ability to bind to PME-1 alone or in complex with PP2A. A compound that is found to effectively bind the PME-1 holoenzyme may be identified as an “inhibitor” of PME-1 activity if it can then be shown that the binding of the compound affects the methylation status, e.g. increased, of PP2A. Such “inhibitors” may then be used to modulate the activity of PP2A in vitro or in vivo. In still other embodiments, such “inhibitors” of PME-1 may be administered to a subject or used as part of a pharmaceutical composition to be administered to individuals in need thereof.

The terms “complementary” or “substantially complementary” as used herein, refers to a compound having a size, shape, charge or any combination of these characteristics that allow the compound to substantially fill contours created by applying an three-dimensional modeling algorithm to a portion of PME-1 alone or in complex with PP2A. A compound that substantially fills without overlapping portions of the various elements that make up PME-1 alone or in complex with PP2A, even if various portions of the space remain unfilled, may be considered “substantially complementary”.

The terms “similar” or “substantially similar” may be used to describe a compound having a size, shape, charge or any combination of these characteristics similar to a compound known to bind PME-1 alone or in complex with PP2A. For example, an identified compound having a similar size, shape, and/or charge to a portion of the C-subunit may be considered “substantially similar” to the C-subunit.

Any inhibitor identified using the techniques described herein, may bind to PME-1 alone or in complex with PP2A with at least about the same affinity of the protein which binds at a selected interface or a known inhibitor to a known binding site, and in certain embodiments, the inhibitor may have an affinity for PME-1 and/or PP2A that is greater than the affinity of the natural or known substrate for PME-1 and/or PP2A. Thus, such inhibitors may bind to PME-1 and/or PP2A and inhibit the activity of PME-1 and/or PP2A, thereby providing methods and compounds for modulating the activity of PME-1 and/or PP2A. Without wishing to be bound by theory, modulation of PP2A may reduce or PP2A mediated serine/threonine dephosphorylation, and modulating the activity of PP2A may provide the basis for treatment of various cell cycle modulation or proliferative disorders including, for example, cancer and autoimmune disease.

Determination of the atomic coordinates of any portion of PME-1 alone or in complex with PP2A may be carried out by any method known in the art. For example, the atomic coordinates provided in embodiments of the invention, or the atomic coordinates provided by other PP2A crystallographic or NMR structures including, but not limited to, crystallographic or NMR data for PME-1, PP2A core, PP2A holoenzyme or individual A, B or C components of PP2A, may be provided to a molecular modeling program and the various portions of PP2A holoenzyme described above may be visualized. In other embodiments, two or more sets of atomic coordinates corresponding to various portions of PME-1 alone or in complex with PP2A may be compared and composite coordinates representing the average of these coordinates may be used to model the structural features of the portion of PME-1 alone or in complex with PP2A under study. The atomic coordinates used in such embodiments may be derived from purified PME-1, PP2A holoenzyme, individual A, B or C subunits, or PP2A bound to other regulatory proteins, substrate proteins, accessory proteins, protein fragments or peptides. In general, atomic coordinates defining a three-dimensional structure of a crystal of PME-1 alone or in complex with PP2A holoenzyme that diffracts X-rays for the determination of atomic coordinates to a resolution of 5 Angstroms or better may be used.

Having defined the structural features of PME-1 alone or in complex with PP2A, mimetics or small molecules substantially complementary to various portions of the PME-1 alone or in complex with PP2A holoenzyme, such as those described above, may be designed. Various methods for molecular design are known in the art, and any of these may be used in embodiments of the invention. For example, in some embodiments, compounds may be specifically designed to fill contours of a portion of PME-1 and/or PP2A at the interfaces between PP2A and PME-1 or in portions of PME-1 and/or PP2A where other factors or substrate proteins interact. In other embodiments, random compounds may be generated and compared to the spatial coordinates such as a portion of PME-1 alone or in complex with PP2A. In still other embodiments, stored spatial coordinates of candidate compounds contained within a database may be compared to the spatial coordinates of a portion of PME-1 alone or in complex with PP2A. In certain embodiments, molecular design may be carried out in combination with molecular modeling.

Methods for performing structural comparisons of atomic coordinates of molecules including those derived from protein crystallography are well known in the art, and any such method may be used in various embodiments to test candidate PME-1 and/or PP2A binding compounds for the ability to bind a portion of PME-1 and/or PP2A. In such embodiments, atomic coordinates of designed, random or stored candidate compounds may be compared against a portion of PME-1 alone or in complex with PP2A or the atomic coordinates of a compound bound to PME-1 alone or in complex with PP2A. In other such embodiments, a designed, random or stored candidate compound may be brought into contact with a surface of PME-1 alone or in complex with PP2A, and simulated hydrogen bonding and/or van der Waals interactions may be used to evaluate or test the ability of the candidate compound to bind the surface of PME-1 alone or in complex with PP2A. Structural comparisons, such as those described in the preceding embodiments may be carried out using any method, such as, for example, a distance alignment matrix (DALI), Sequential Structure Alignment Program (SSAP), combinatorial extension (CE) or any such structural comparison algorithm. Compounds that appear to mimic a portion of the PME-1 alone or in complex with PP2A under study or a compound known to bind PME-1 alone or in complex with PP2A, such as, for example, a substrate protein, or that are substantially complementary and have a likelihood of forming sufficient interactions to bind to PME-1 alone or in complex with PP2A may be identified as a potential PP2A holoenzyme binding compound.

In some embodiments, compounds identified as described above may conform to a set of predetermined variables. For example, in one embodiment, the atomic coordinates of an identified PME-1 alone or in complex with PP2A binding compound when compared with a PME-1 alone or in complex with PP2A binding compound or a subunit of PME-1 alone or in complex with PP2A using one or more of the above structural comparison methods may deviate from an rmsd of less than about 10 angstroms. In another embodiment, the atomic coordinates of the compound may deviate from the atomic coordinates of PME-1 alone or in complex with PP2A by less than about 2 angstroms. In still another embodiment, the identified PME-1 alone or in complex with PP2A binding compound may include one or more specific structural features known to exist in a PME-1 alone or in complex with PP2A binding compound or a subunit of PME-1 alone or in complex with PP2A, such as, for example, a surface area, shape, charge distribution over the entire compound or a portion of the identified compound.

Compounds identified by the various methods embodied herein may be synthesized by any method known in the art. For example, identified compounds may be synthesized using manual techniques or by automation using in vitro methods such as, various solid state or liquid state synthesis methods. Direct peptide synthesis using solid-phase techniques is well known and utilized in the art (see, e.g., Stewart et al., Solid-Phase Peptide Synthesis, W. H. Freeman Co., San Francisco, Calif. (1969); Merrifield, J. Am. Chem. Soc., 85:2149-2154 (1963)). Automated synthesis may be accomplished, for example, using an Peptide Synthesizer using manufacturer's instructions. Additionally, in some embodiments, one or more portion of the PME-1 and/or PP2A modulators described herein may be synthesized separately and combined using chemical or enzymatic methods to produce a full length modulator.

Compounds identified using various methods of embodiments of the invention may be further tested for binding to PME-1 alone or in complex with PP2A and/or to determine the compound's ability to inhibit activity of PME-1 and/or PP2A or modulate the activity of PME-1 and/or PP2A by, for example, testing for pTyr activity or testing the candidate compound for binding to PME-1 and/or PP2A. Such testing may be carried out by any method. For example, such methods may include contacting a known substrate with an identified compound and detecting binding to PME-1 and/or PP2A by a change in fluorescence in a marker or by detecting the presence of the bound compound by isolating the PME-1 and/or PP2A candidate compound complex and testing for the presence of the compound. In other embodiments, PME-1 and/or PP2A activity may be tested by, for example, isolating a substrate peptide that has or has not been methylated or phosphorylated or isolating a PME-1 and/or PME-1 in complex with PP2A that has been contacted with the candidate compound. Such methods are well known in the art and may be carried out in vitro, in a cell-free assay, or in vivo, in a cell-culture assay.

Embodiments of the invention also include pharmaceutical compositions including inhibitors that bind to PME-1 and/or PP2A and inhibit PME-1 and/or PP2A activity or compounds that are identified using methods of embodiments described herein above and a pharmaceutically acceptable carrier or excipient. Such pharmaceutical compositions may be administered to an individual in an effective amount to alleviate conditions associated with PP2A activity.

Various embodiments of the invention also include a system for identifying a PME-1 and/or PP2A modulator. Such systems may include a processor and a computer readable medium in contact with the processor. The computer readable medium of such embodiments may at least contain the atomic coordinates of PME-1 alone or in complex with PP2A. In some embodiments, the computer readable medium may further contain one or more programming instructions for comparing at least a portion of the atomic coordinates of PME-1 alone or in complex with PP2A with atomic coordinates of candidate compounds included in a library of compounds. In other embodiments, the computer readable medium may further contain one or more programming instructions for designing a compound that mimics at least a portion of PME-1 alone or in complex with PP2A or that is substantially complementary to a portion of PME-1 alone or in complex with PP2A. In still other embodiments, the computer readable medium may contain one or more programming instructions for identifying candidate compounds or designing a compound that mimics a portion of PME-1 alone or in complex with PP2A within one or more user defined parameters. For example, in some embodiments, a compound may include a charged molecule at a particular position corresponding to one or more positions within the atomic coordinates of PME-1 alone or in complex with PP2A, and in other embodiments, the compound may deviate from the carbon backbone or surface model representation of PME-1 alone or in complex with PP2A by, for example, an rmsd of less than about 10 Å. In still other embodiments, a user may determine the size of a candidate compound or the portion of PME-1 alone or in complex with PP2A that is utilized in identifying mimetic candidate compounds. Further embodiments may include one or more programming instructions for simulating binding of an identified candidate compound to PME-1 alone or in complex with PP2A or a portion of PME-1 alone or in complex with PP2A. Such embodiments may be carried out using any method known in the art, and may provide an additional in silico method for testing identified candidate compounds.

Some embodiments of invention described herein may encompasses pharmaceutical compositions comprising a therapeutically effective amount of an inhibitor in dosage form and a pharmaceutically acceptable carrier, wherein the compound inhibits methylesterase activity of PME-1 and/or the phosphotyrosyl or phosphoserosyl activity of PP2A. In another embodiment, such compositions comprise a therapeutically effective amount of an inhibitor in dosage form and a pharmaceutically acceptable carrier in combination with a chemotherapeutic and/or radiotherapy, wherein the inhibitor inhibits the methylesterase activity of PME-1 and/or the phosphotyrosyl or phosphoserosyl activity of PP2A, promoting apoptosis and enhancing the effectiveness of the chemotherapeutic and/or radiotherapy. In various embodiments of the invention, a therapeutic composition for modulating PME-1 and/or PP2A activity comprises a therapeutically effective amount of a PP2A inhibitor.

Some embodiments of the invention also include methods for treating a patient having a condition characterized by aberrant cell growth, wherein administration of a therapeutically effective amount of a PME-1 and/or PP2A inhibitor is administered to the patient, and the inhibitor binds to PME-1 and/or PP2A modulates cell growth. The method may further include the concurrent administration of a chemotherapeutic agent, such as, but not limited to, alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-inflammatory compounds. As used herein, “concurrent administration” may be administration prior to, substantially simultaneous with, simultaneous with or following administration of the PME-1 and/or PPSA inhibitor.

The PME-1 and/or PP2A inhibitors of the invention may be administered in an effective amount. In certain embodiments, an “effective amount” is an amount of a preparation that alone, or together with further doses, produces the desired response. This may involve only slowing the progression of the disease temporarily, although it may involve halting the progression of the disease permanently or delaying the onset of or preventing the disease or condition from occurring. This can be monitored by routine methods known and practiced in the art. Generally, doses of active compounds may be from about 0.01 mg/kg per day to about 1000 mg/kg per day, and in some embodiments, the dosage may be from about 50-500 mg/kg. In various embodiments, the compounds of the invention may be administered intravenously, intramuscularly, or intradermally, and in one or several administrations per day. The administration of inhibitors can occur simultaneous with, subsequent to, or prior to chemotherapy or radiation.

In general, routine experimentation in clinical trials will determine specific ranges for optimal therapeutic effect for each therapeutic agent and each administrative protocol and administration to specific patients will be adjusted to within effective and safe ranges depending on the patient's condition and responsiveness to initial administrations. However, the ultimate administration protocol will be regulated according to the judgment of the attending clinician considering such factors as age, condition and size of the patient, the potency of the PME-1 and/or PP2A inhibitor administered, the duration of the treatment and the severity of the disease being treated. For example, a dosage regimen of a PME-1 and/or PP2A inhibitor to, for example, reduce cellular proliferation or induce apoptosis can be oral administration of from about 1 mg to about 2000 mg/day, preferably about 1 to about 1000 mg/day, more preferably about 50 to about 600 mg/day. In certain embodiments, the dosage may be administered once daily or in divided doses, such as in two, three to four divided doses. Intermittent therapy (e.g., one week out of three weeks or three out of four weeks) may also be used.

In the event that a response in a subject is insufficient at the initial doses applied, higher doses (or effectively higher doses by a different, more localized delivery route) may be employed to the extent that the patient's tolerance permits. Multiple doses per day are contemplated to achieve appropriate systemic levels of compounds. Generally, a maximum dose is used, that is, the highest safe dose according to sound medical judgment. However, an individual patient may insist upon a lower dose or tolerable dose for medical reasons, psychological reasons or for virtually any other reason.

Some embodiments of the invention also include a method of treating a patient with cancer or an autoimmune disease by promoting apoptosis, wherein administration of a therapeutically effective amount of one or more PME-1 and/or PP2A inhibitors, and the PME-1 and/or PP2A inhibitor inhibit the methylesterase activity of PME-1 and/or the phosphotyrosyl or phosphoserosyl activity of PP2A. The method may further include concurrent administration of a chemotherapeutic agent including, but not limited to, alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-inflammatory compounds.

A variety of administration routes are available. The particular mode selected will depend upon the severity of the condition being treated and the dosage required for therapeutic efficacy. The methods of the invention may be practiced using any mode of administration that is medically acceptable, meaning any mode that produces effective levels of active compounds without causing clinically unacceptable adverse effects. Such modes of administration include, but are not limited to, oral, rectal, topical, nasal, intradermal, inhalation, intra-peritoneal, or parenteral routes. The term “parenteral” includes subcutaneous, intravenous, intramuscular, or infusion. Intravenous or intramuscular routes may be particularly suitable for purposes of the present invention.

In one aspect of the invention, a PME-1 and/or PP2A inhibitor as described herein, with or without additional biological or chemotherapeutic agents or radiotherapy, does not adversely affect normal tissues while sensitizing aberrantly dividing cells to the additional chemotherapeutic/radiation protocols. While not wishing to be bound by theory because the PME-1 and/or PP2A inhibitors specifically target PME-1 and/or PP2A, marked and adverse side effects may be minimized. In certain embodiments, the composition or method may be designed to allow sensitization of the cell to chemotherapeutic agents or radiation therapy by administering the ATPase inhibitor prior to chemotherapeutic or radiation therapy.

The term “pharmaceutically-acceptable carrier” as used herein, means one or more compatible solid or liquid fillers, diluents or encapsulating substances which are suitable for administration into a human. The term “carrier” or “excipient” denotes an organic or inorganic ingredient, natural or synthetic, with which the active ingredient is combined to facilitate the application. The components of the pharmaceutical compositions are also capable of being co-mingled with the molecules of the present invention and with each other, in a manner such that there is no interaction which would substantially impair the desired pharmaceutical efficacy.

The delivery systems that may be used in embodiments of the invention are designed to include time-released, delayed release or sustained release delivery systems such that the delivery of the PP2A inhibitors occurs prior to, and with sufficient time, to cause sensitization of the site to be treated. For example, a PME-1 and/or PP2A inhibitor may be used in conjunction with radiation and/or additional anti-cancer chemical agents. Such systems can avoid repeated administrations of the PP2A inhibitor compound, increasing convenience to the subject and the physician, and may be particularly suitable for certain compositions of the present invention.

Many types of release delivery systems are available and known to those of ordinary skill in the art including, but not limited to, polymer base systems, such as, poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems including, for example: lipids including sterols, such as cholesterol, cholesterol esters and fatty acids or neutral fats, such as mono-, di- and tri-glycerides; hydrogel release systems; sylastic systems; peptide based systems; wax coatings; compressed tablets using conventional binders and excipients; partially fused implants; and the like. Specific examples include, but are not limited to: erosional systems in which the active compound is contained in a form within a matrix such as those described in U.S. Pat. Nos. 4,452,775, 4,667,014, 4,748,034, and 5,239,660 and diffusional systems in which an active component permeates at a controlled rate from a polymer, such as described in U.S. Pat. Nos. 3,832,253, and 3,854,480. In addition, pump-based hardware delivery systems can be used, some of which are adapted for implantation.

In certain embodiments, use of a long-term sustained release implant may be desirable. Long-term release is used herein, and means that the implant is constructed and arranged to deliver therapeutic levels of the active ingredient for at least about 30 days, and preferably about 60 days. Long-term sustained release implants are well-known to those of ordinary skill in the art and include some of the release systems described above.

The pharmaceutical compositions may conveniently be presented in unit dosage form and may be prepared by any of the methods well known in the art of pharmacy. All methods include the step of bringing the active agent into association with a carrier that constitutes one or more accessory ingredients. In general, the compositions may be prepared by uniformly and intimately bringing the active compound into association with a liquid carrier, a finely divided solid carrier, or both and then, if necessary, shaping the product.

Compositions suitable for parenteral administration conveniently include a sterile aqueous preparation of an ATPase inhibitor which is preferably isotonic with the blood of the recipient. This aqueous preparation may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile fixed oils are conventionally employed as a solvent or suspending medium. For this purpose, any bland fixed oil may be employed including synthetic mono- or di-glycerides. In addition, fatty acids, such as oleic acid, may be used in the preparation of injectables. Carrier formulation suitable for oral, subcutaneous, intravenous, intramuscular, etc. administrations can be found, for example, in Remington's Pharmaceutical Sciences, Mack Publishing Co., Easton, Pa. which is incorporated herein in its entirety by reference thereto.

### EXAMPLES

### Example 1

### Structure of PME-1

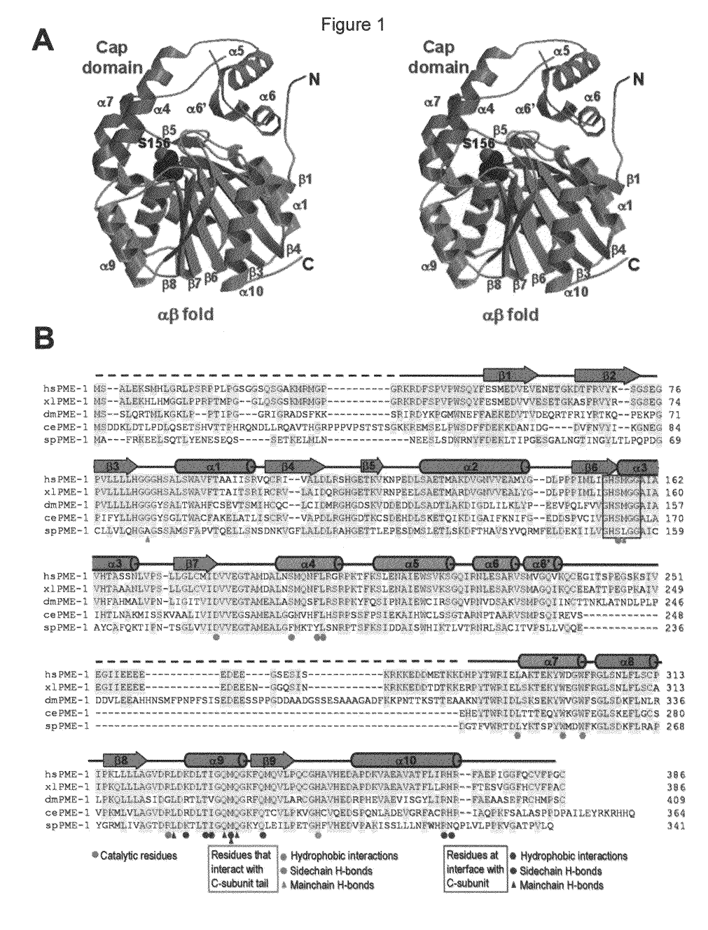

We first sought to determine the crystal structure of PME-1. The full-length PME-1 failed to crystallize, likely due to the flexible amino-terminal sequences and an internal Glu-rich loop. Removal of these flexible moieties by limited proteolysis generated a core enzyme that exhibited a similar activity as the full-length PME-1 (data not shown). We obtained crystals of this PME-1 core enzyme. The structure was determined by selenomethionine multi-wavelength anomalous dispersion (MAD) and refined to 2.1 Å resolution (FIG. 1 and Table 1). To our knowledge, PME-1 represents the first structure of a eukaryotic protein methylesterase. The overall structure of the PME-1 core enzyme belongs to the α/β hydroxylase superfamily. The α/β fold contains a central, 9-stranded β-sheet, surrounded by two α-helices on one side (α1 & α10) and four α-helices on the other (α2, α3, α8, and α9) (FIG. 1). The putative catalytic residue Ser156, from the conserved motif GxSxxG, is located at the turn between strand β6 and helix α3. Four additional α-helices form a cap domain above Ser156. The active site pocket is located between the cap domain and the α/β fold (FIG. 1A).

In the absence of PP2A, the active site of PME-1 appears to exist in an inactive conformation that may not support catalysis, because Ser156 is not within hydrogen bond distance of any histidine residue. Supporting this structural observation, prior treatment of PME-1 with PMSF, a serine protease inhibitor, did not result in any detectable reduction of methylesterase activity towards the PP2A core enzyme. In addition, PME-1 does not bind to and, exhibits no detectable methylesterase activity towards, a carboxylmethylatedZ peptide TPDYFL309 (Lee et al., 1996). Collectively, these observations suggest that PME-1 must undergo an activation process, which is likely mediated by binding to the PP2A core enzyme.

### Structure of the PP2A-PME-1 Heterotrimeric Complex

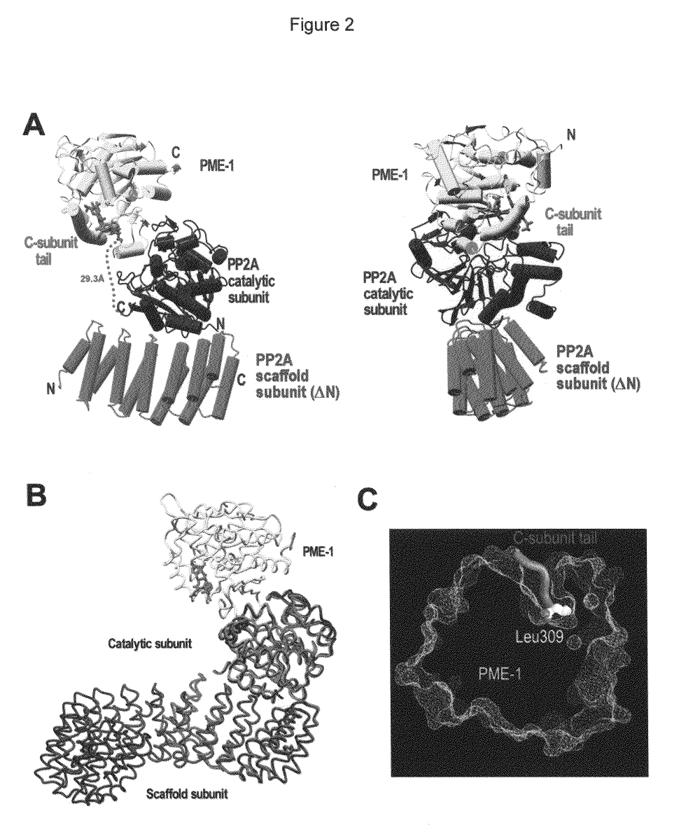

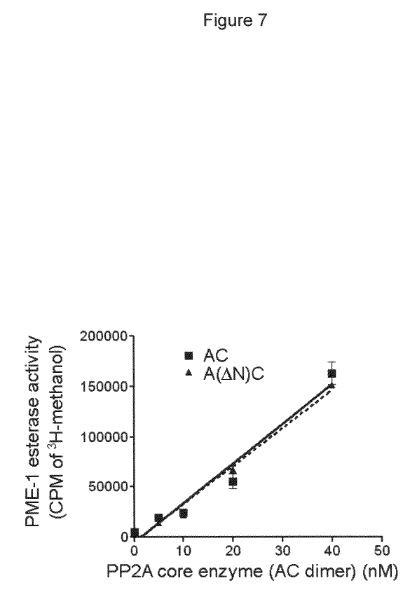

Despite extensive effort, a heterotrimeric complex between PME-1 and the full-length PP2A core enzyme eluded crystallization. This was likely due to the flexible nature of the A subunit, a HEAT repeat protein that is known for heterogeneous conformations. To alleviate this problem, we progressively deleted the amino-terminal HEAT repeats that are dispensable for binding to the C subunit and PME-1 (Ruediger et al., 1994; Ruediger et al., 1992; Xing et al., 2006). One specific variant of the A subunit, containing HEAT repeats 1 and 11-15 (AΔN), formed a stable heterotrimeric complex with the C subunit and PME-1. The methylesterase activity of PME-1 on the PP2A core enzyme involving the AΔN variant was nearly identical to that on the PP2A core enzyme involving the full-length A subunit (FIG. 7). This result suggests that deletion of HEAT repeats 2-10 in the A subunit has no detectable impact on the interaction between PME-1 and the PP2A core enzyme. The PP2A core enzyme involving AΔN was first methylated by LCMT1 and then incubated with the catalytic mutant of PME-1 (S156A) to reconstitute a heterotrimeric PP2A-PME-1 complex. The heterotrimeric complex was crystallized and the structure was determined by molecular replacement at 2.8 Å resolution (Table 1 and FIG. 2).

The structure of the PP2A-PME-1 complex showed that PME-1 binds exclusively to the C subunit of the PP2A core enzyme (FIG. 2A). Structural alignment of the PP2A/PME-1 complex with the PP2A core enzyme indicates that PME-1 makes no direct interactions with the A subunit (FIG. 2B). As previously observed, the C subunit sits on the ridge of HEAT repeats 11-15 in the A subunit (FIG. 2A). Inclusion of HEAT repeat 1 in the AΔN subunit proved to be essential for its solubility and crystallization of the heterotrimeric complex. In the crystals, HEAT repeat 1 stacks against HEAT repeat 11 to insulate the hydrophobic surfaces in both repeats. PME-1 binds to the structural elements surrounding the active site of the C subunit. This arrangement creates an extended, linear architecture, with a height of approximately 100 Å (FIG. 2A).

The active site pocket of PME-1 is located close to the C subunit, with an orientation that is conducive to binding by the carboxyl-methylated peptide from the C subunit. In the structure, the carboxy-terminal 6 amino acids T₃₀₄PDYFL₃₀₉ (SEQ ID NO: 5) of the C subunit are bound in the active site pocket of PME-1 (FIG. 2C). The flexible sequences spanning Pro293-Thr304 are disordered in the crystals and have no electron density. Pro293 is separated from Thr304 by approximately 30 Å (FIG. 2A); the 11 peptide bonds absent in the structure between these two residues could reach a distance of as much as 36 Å in an extended conformation.

### Interface Between PME-1 and the C Subunit

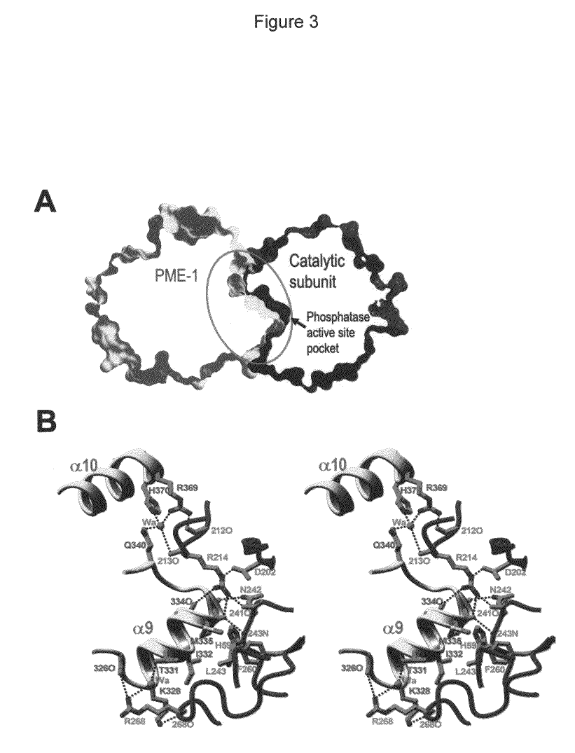

PME-1 appears to bind directly to the active site of the C subunit. These two proteins stack closely against each other, creating an S-shaped interface (FIG. 3A). This interface is dominated by hydrogen bonds (H-bonds), which are buttressed by additional van der Waals contacts. Together, these interactions result in the burial of approximately 1800 Å2 exposed surface area. Residues from helix α9 of PME-1, and to a lesser extent helix α10, mediate these interactions.

Examination of the interface revealed three networks of H-bonds (FIG. 3B). In the center of the interface, Arg214 of the C subunit donates two inter-molecular H-bonds, to the carbonyl oxygen atoms of residues 334 and 335 in PME-1, and two intramolecular H-bonds, one to Asp202 and the other to carbonyl oxygen of residue 241 in the C subunit. These interactions are strengthened by two additional inter-molecular H-bonds between carbonyl oxygen atoms of residues 335/336 in PME-1 and amide nitrogen atoms of residues 243/242 in the C subunit. These H-bonds are stabilized by a patch of van der Waals interactions, between residues Ile332 and Met335 in PME-1 and residues His59, Leu243 and Phe260 in the C subunit. Of these residues, His59 was shown to bind to metal ions in the active site of the PP2A core enzyme (Xing et al., 2006).

In the periphery of the interface, two H-bond networks stabilize both ends of the S-shaped interface. In one network, three H-bonds were formed between Arg268 of the C subunit and several residues at the amino-terminus of helix α9 in PME-1 (FIG. 3B). In the other network, a water molecule mediates hydrogen bonds among residue 213 in the C subunit and the side chains of Gln340, Arg369 and His370 in PME-1. Arg369 forms an additional hydrogen bond to residue 212 of the C subunit.

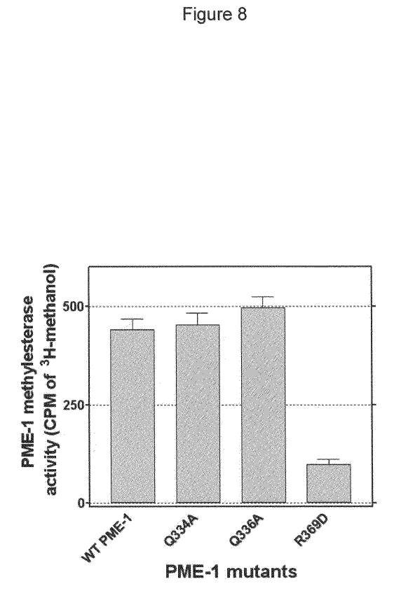

To corroborate these structural observations, we performed a mutational analysis on PME-1 FIG. 8. We generated three missense mutations in PME-1 and assayed their impact on the methylesterase activity of PME-1. Because these mutations do not involve residues at the active site of PME-1, a decrease in the methylesterase activity likely reflects a weakened binding affinity between PP2A and the specific PME-1 mutant. In the structure, the side chain of Arg369 participates in multiple inter-molecular hydrogen bonds with PP2A; thus its mutation to Asp is expected to reduce the binding affinity between PME-1 and PP2A. Consistent with this prediction, R369D in PME-1 resulted in marked reduction of its methylesterase activity FIG. 8. In contrast, the side chains of Gln334 and Gln336 in PME-1 do not appear to be involved in any interaction with PP2A; their mutations are predicted not to affect the PME-1-PP2A interaction and thus did not appear to have any effect on the methylesterase activity FIG. 8.

### Recognition of Substrate by PME-1

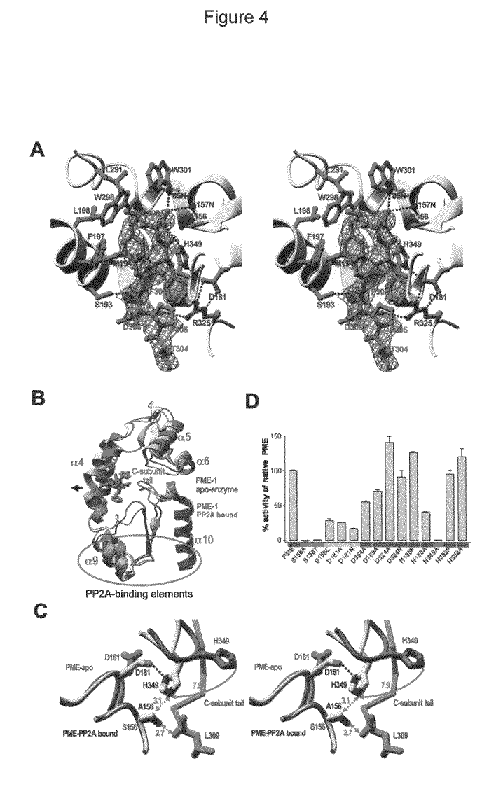

The carboxy-terminal residues of the C-subunit bind to the active site pocket of PME-1 (FIG. 4A). The interface contains extensive van der Waals contacts, with three inter molecular H-bonds contributing to the specificity of recognition. Collectively, these interactions result in the burial of approximately 1130 Å² exposed surface area.

On one side of the active site pocket, the side chains of Tyr307 and Leu309 in the C subunit are nestled in a hydrophobic groove formed by six residues in the cap domain of PME-1: Met194, Phe197 and Leu198 in helix α4, and Leu291, Trp298 and Trp301 in helix α7 (FIG. 4A). On the other side of the pocket, a network of H-bonds stabilizes the conformation of the catalytic triad and helps coordinate the substrate (FIG. 4A). These H-bonds involve residues Arg325, Asp181, His349, backbone amides of residues 85 and 157 of PME-1, and the carboxylate group of Leu309 in the C subunit. In particular, Asp181 accepts an H-bond from His 349. Ala156, which corresponds to the catalytic residue Ser156, is located close to the carboxylate group of Leu309 and within close proximity of His349 in PME-1. These observations suggest that, in contrast to free PME-1, the active site residues in the PP2A-bound PME-1 may exist in an active conformation, poised to catalyze the bound substrate.

### Activation of PME-1

Structural comparison between free PME-1 and the PP2A-bound PME-1 reveals that the substrate peptide would overlap significantly with helix α4 of the free PME-1 (FIG. 4B). This analysis could explain why free PME-1 does not bind to the isolated carboxylmethylated peptide TPDYFL₃₀₉ of the C subunit. This observation, together with the inactive conformation of the catalytic triad residues in free PME-1, could also explain why free PME-1 exhibits no detectable methylesterase activity for the isolated peptide substrate (Lee et al., 1996).

The interactions between PME-1 and PP2A result in two striking consequences. The first consequence is the activation of PME-1. The specific interactions between PME-1 and the C subunit induce marked conformational rearrangements surrounding the active site region of PME-1, resulting in its activation (FIG. 4C). In particular, extensive interactions of helix α9 in PME-1 with the C subunit cause α9 to shift towards helix α4, which forces α4 to bend (FIG. 4B). These structural changes result in the widening of the active site pocket in PME-1 that accommodates the substrate peptide.

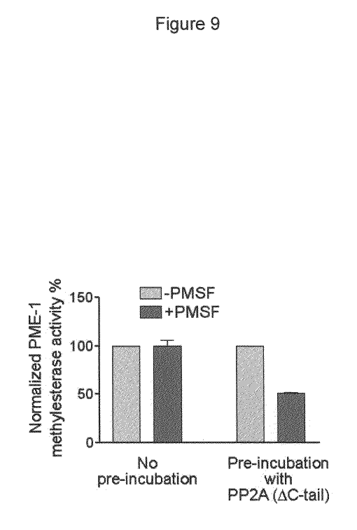

These changes also cause the catalytic triad residues to rearrange into an active conformation (FIG. 4C). Remarkably, the side chain of His349 swings approximately 8 Å to hydrogen bond with Asp181 and to be within H-bond distance of the catalytic residue 156. The carboxylate group of Leu309 in the substrate peptide is in close proximity of the catalytic residue and in an appropriate orientation for catalysis. Corroborating these structural observations, PME-1 was only inactivated by PMSF after PME-1 was pre-incubated with a PP2A core enzyme involving truncation of the carboxyterminus of the catalytic subunit FIG. 9.

Our structural analysis identifies Ser156, His349, and Asp181 as the catalytic triad residues. Consistent with this analysis, mutation of Ser156 or His349 to Ala abolished the methylesterase activity, whereas mutation of Asp181 markedly reduced the esterase activity by 75 percent or more (FIG. 4D). In contrast, mutations of other histidine and aspartate residues near the active site have much less impact on the esterase activity of PME-1 (FIG. 4D).

### Inactivation of PP2A by PME-1

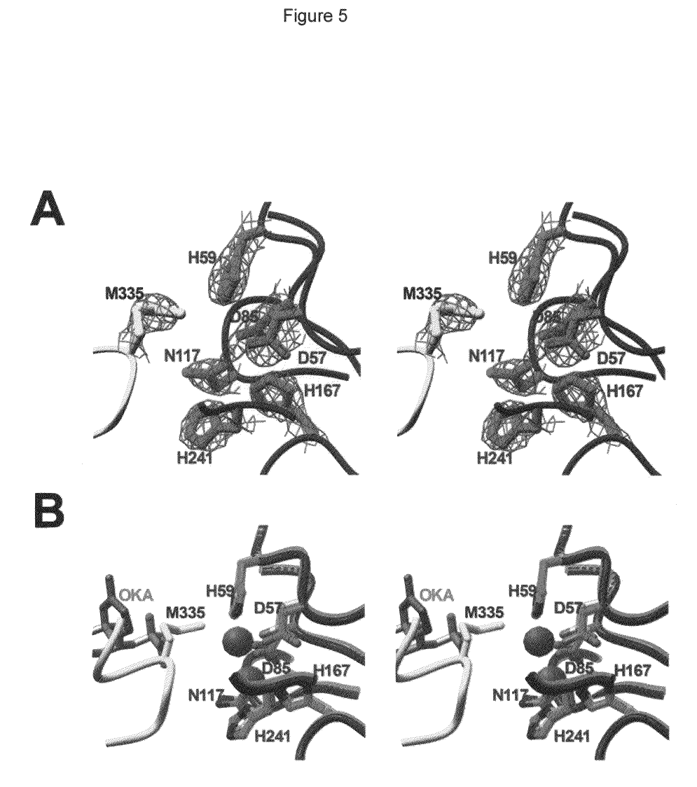

The second striking consequence following binding of PME-1 to the PP2A core enzyme is the displacement of the metal ions from the active site of the C subunit (FIG. 5A). The two manganese atoms in the C subunit of the PP2A core enzyme were dislodged and gone in the PP2A-PME-1 complex. Consequently, the PP2A core enzyme is inactivated by PME-1.

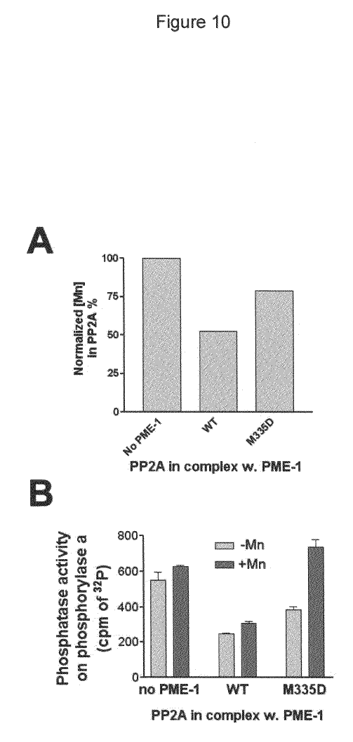

Met335 in PME-1, a hydrophobic amino acid highly conserved in other PME orthologs (FIG. 1B), appears to play an important role in displacing the metal atoms. Comparison with the structure of the PP2A core enzyme reveals that the side chain of Met335 penetrates deeper into the active site of the C subunit than the acidic head group of okadaic acid (FIG. 5B). The hydrophobic nature of Met335 may also discourage the re-loading of metal ions into the active site. Consistent with this interpretation, mutation of Met335 to aspartate in PME-1 led to decreased ability to remove the metal ions, and the PP2A-PME-1-M335D complex became readily activated by manganese (FIG. 10). Supporting our structural observation, an inactive population of PP2A was previously shown to be stably associated with PME-1 in cells that could not be activated by manganese (Longin et al., 2004).

### DISCUSSION

Despite intense study of PME-1 in the last decade, the mechanisms of substrate binding, catalysis, and function remained enigmatic. Our structural analysis of PME-1 by itself and in complex with PP2A showed that PME-1 appears to only be activated upon binding to PP2A. This property ensures the specificity of the PME-1-associated methylesterase activity for PP2A. Activation of PME-1 entails two major conformational rearrangements: opening of the active site pocket through bending of helix α4 and alignment of the catalytic triad residues into an active conformation. These observations provide a satisfactory explanation to the questions of why a synthetic peptide alone could not be the substrate or inhibitor of PME-1 and why the esterase activity could not be inhibited by PMSF treatment. The structural observation that PME-1 binds directly to the PP2A active site, overlapping the binding sites for phosphatase inhibitors okadaic acid and microcystin LR, explains why these inhibitors blocked the methylesterase activity of PME-1.

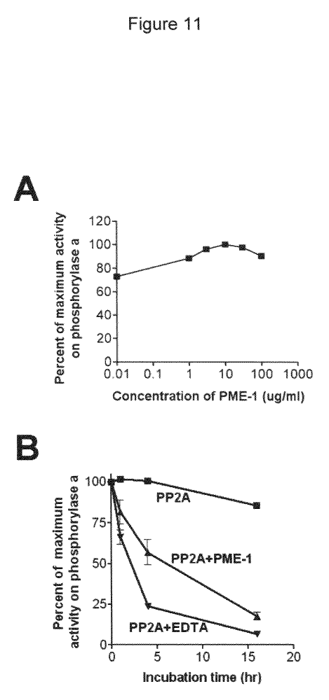

Our structural observation clearly indicates that PME-1 has the ability to inactivate the phosphatase activity of PP2A. However, previous studies showed that serine/threonine phosphatase activity of PP2A could not be blocked by PME-1. While not wishing to be bound by theory, we believe that these seemingly conflicting observations reflect the kinetic nature of the metal ion removal. Although binding of PME-1 to the PP2A core enzyme occurs instantaneously, removal of the metal atoms takes time. The PP2A core enzyme, upon initial incubation with various concentrations of PME-1, exhibited full phosphatase activity towards the substrate phosphorylase a (FIG. 11A). A prolonged incubation time was required for substantial loss of the phosphatase activity in vitro (FIG. 11B). It should be noted, however, that these experiments were performed in the presence of 100 μM manganese ion and the free metal ions may slow down the process of PME-1-mediated metal removal from the catalytic subunit of PP2A. In cells, the dislodged metal ions are presumably swept up by metal-binding proteins and no longer available for reloading into PP2A. This analysis suggests that the slow kinetics might be accelerated in cells. Consistent with this analysis, incubation of PP2A with EDTA resulted in marked reduction of phosphatase activity, presumably due to metal chelation (FIG. 11B).

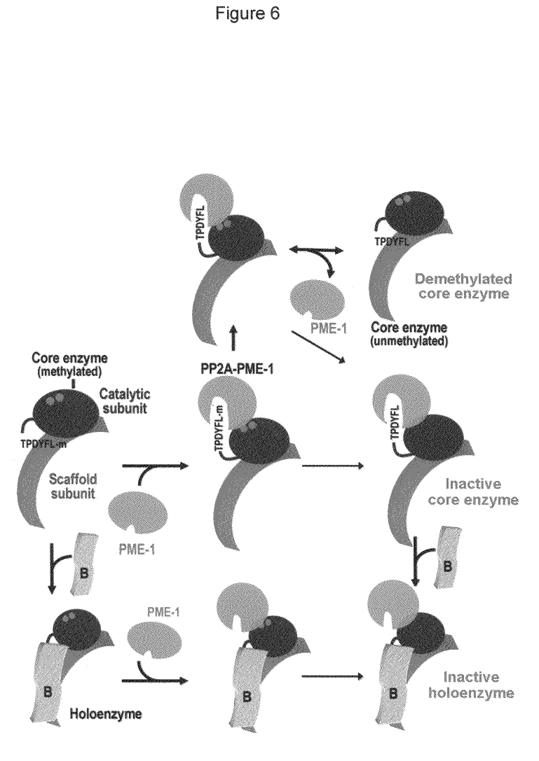

It is important to note that, because PME-1 does not bind to the A subunit, PME-1 binding to the PP2A core enzyme is unlikely to affect the interactions between the regulatory subunits and the PP2A core enzyme. In other words, PME-1 is unlikely to dissociate pre-assembled holoenzymes. Nonetheless, PME-1 might still be able to exert negative regulation on the pre-assembled holoenzyme through removal of its catalytic metal ions (FIG. 6).

Earlier experimental evidence showed that methylation of the carboxy-terminus of the C subunit may play an important role in the formation of PP2A holoenzymes. Recent observations suggest that methylation of the carboxy-terminal Leu309 in a conserved TPDYFL309 motif of the C subunit may enhance the affinity of the PP2A core enzyme for some, but not all, regulatory subunits. It should be noted, however, enhanced affinity only represents one strategy by which methylation may facilitate assembly of the PP2A holoenzymes. Alternatively, methylation could serve as a targeting signal to bring together all components that are required for holoenzyme assembly. Investigation of this hypothesis requires a separate mechanistic study.

The interaction between PME-1 and PP2A is subject to regulation by an array of other PP2A-binding Xing et al—15—factors, including but not limiting to LCMT1 and PTPA. The opposing roles of PME-1 and PTPA might form a regulatory circuit of PP2A inactivation and activation, presumably through metal removal and reloading. Furthermore, the stable complex between PP2A and PME-1 blocks LCMT1-catalyzed methylation (data not shown). Deletion of PTPA homologs in yeast, rrd1/rrd2, resulted in elevated levels of stable PP2A-PME-1 complexes, accompanied by decreased methylation. In this regard, our structure of the PP2A-PME-1 complex might represent an important intermediate of PP2A biogenesis that could only be methylated after activation in cells. The dual roles of PME-1 in counteracting the function of PTPA and LCTM1 provide a mechanism for coupling PP2A activation with methylation. Our structural observations provide mechanistic insights into the interplay among PP2A methylation, holoenzyme assembly, and its activation.

### Example 2

### Experimental Procedures

Protein preparation and assembly of PP2A-PME-1 complex All constructs and point mutations were generated using a standard PCR-based cloning strategy. Full-length PME1 (1-386) and all the mutants were cloned in pET15b vector (Invitrogen) and were overexpressed at room temperature in E. coli strain BL21(DE3). The soluble fraction of the E. coli cell lysate was purified by the Ni-NTA resin (Qiagen) to homogeneity and further fractionated by ion exchange chromatography (Source 15Q, Amersham). PME-1 with methionine residues replaced by selenium methionine was expressed by growing the bacteria in a minimum medium containing selenium methionine and purified in a similar way.

To facilitate crystallization of the PP2A-PME-1 complex, a PME-1 construct, containing residues 39-238, a small linker ‘EGK’ and residues 284-376, was made based on the crystal structure of PME-1, which reserves all the structural moieties for the esterase activity and PP2A recognition. Cloning, expression, purification of PP2A Aα subunit (AΔN), Cα subunit followed similar procedures as described previously. The PP2A core enzyme, involving the full-length Cα and AΔN, was assembled as described. The PP2A core enzyme was methylated by a PP2A-specific leucine carboxyl methyltransferase (LCMT1) in the presence of S-adenosyl methionine (SAM) (see below). Following complete methylation, the PP2A core enzyme was incubated with an excess amount of PME-1 containing an inactive mutation (S156A). The PP2A-PME-1 complex was purified to homogeneity by gel-filtration chromatography.

### Limited Proteolysis of PME-1 and Characterization of the PME-1 Enzyme Core

To facilitate crystallization of PME-1, a structural core of PME-1 was generated by incubation of the full length PME-1 with 0.5 mg/ml trypsin for 20 minutes on ice. The enzyme core that contains two fragments of PME-1, 28 and 8 kDa (residues 39-248 and 273-386, respectively) was purified by the anion exchange chromatography (Source 15Q, Amersham). Using the methylesterase activity assay (see below), this PME-1 enzyme core exhibited a similar activity as that of the full-length PME-1 (data not shown).

### Crystallization and Data Collection

The diffracting crystals of PME-1 enzyme core were grown at 4° C. by the hanging-drop vapor-diffusion method by mixing the enzyme (15 mg/ml) with an equal volume of reservoir solution containing 24-26% Jeffamine-2001 (v/v), 200 mM sodium chloride, and 5 mM DTT. The crystals appeared after 1-2 days and reached a maximum size within one week. The crystals are in the space group P3121, with unit cell parameters a=b=82.5 Å, c=90.8 Å, α=β=90°, γ=120°, and contain one protein molecule in each asymmetric unit. The crystals were equilibrated in a cryoprotectant buffer containing the reservoir buffer with 34% (v/v) Jeffamine-2001 and were flash frozen in a cold nitrogen stream at −170° C. A complete 2.0 Å native data set and a complete 2.6 Å selenomethionine MAD dataset were collected at NSLS beamline X25 and processed using the software Denzo and Scalepack (Otwinowski, 1997).

Diffracting crystals of the PP2A-PME-1 complex were grown at room temperature by the hanging-drop vapor-diffusion method by mixing the protein (6 mg/ml) with an equal volume of reservoir solution containing 23-25% PEG3350 (v/v), 100 mM ammonium citrate, and 5 mM DTT. The initial crystals appeared after 5 days, which was used for microseeding thereafter to facilitate nucleation; and the crystals were grown to a maximum size in 2-5 days. The crystals belong to the space group C2, with a=129.3 Å, b=54.8 Å, c=125.2 Å, α=90°, β=111°, γ=90°, and contain one complex per asymmetric unit. Crystals were equilibrated in a cryoprotectant buffer containing reservoir buffer with 34% PEG3350 (v/v), 20% glycerol (v/v) and 50 μM MnCl2, followed by dehydration for 2 hrs and flash frozen as described above. The native datasets were collected at NSLS beamline X29 and processed as described earlier.

### Structure Determination

The structure of human PME-1 was determined by selenium MAD (Hendrickson et al., 1988). Selenium atom locations and initial MAD phases were determined using the SHELX program suite (Sheldrick, 1991), and phases were subsequently improved with the program SHARP to a maximum resolution of 2.6 Å. The experimental map was of sufficient quality that it was possible to automatically build most of the structure using the program ARP/wARP (Perrakis, 1999) using the MAD phases with the incorporation of the native data to 2.0 Å resolution. The refinement was subsequently completed using several rounds of manual model building using the program 0 (Jones et al., 1991) and the refinement using program CNS (Brunger et al., 1998). The structure is refined to 2.1 Å resolution and free and working R factors are 21.9% and 18.2%, respectively.

The structure of human PP2A-PME-1 complex was determined by molecular replacement using three models: Cα (residues 6-293), Aα with heat repeats 11-15 (residues 400-589) from the structure of the PP2A core enzyme (accession code 2IE3) (Xing et al., 2006), and the structure of PME-1. The three proteins in the complex were located using the program PHASER (McCoy et al., 2005). Model building was performed using 0 (Jones et al., 1991) and refined using REFMAC restraints with TLS (Winn et al., 2003), and weights adjusted on the basis of R-free. Three TLS groups were used; one for each subunit; and the TLS parameters was refined in early cycles, and remained the same for later stage of refinements. The final atomic model of the PP2A-PME-1 complex has been refined to 2.8 Å resolution and free and working R factors are 26.4% and 19.5%, respectively.

### PME-1 Methylesterase Activity Assay

The methylesterase activity of PME-1 and its mutants was tested by a cycled methylation and demethylation assay. Briefly, radio-labeled 3H-SAM is mixed with 7.2 μM PP2A core enzyme and 0.2 μM LCMT1 to generate methylated PP2A. Release of H3-methanol was initiated by addition of the full-length PME-1, mutants or the PME-1 core. Concentration dependent PME enzyme activity was determined by the counts of H3-methanol after 2.5 hours or overnight incubation of the reaction mixture at 37° C.

### Methylation of PP2A Core Enzyme by LCMT1

LCMT1 and PP2A core enzyme, at a 1:2 molar ratio, was incubated on ice. Methylation was initiated by addition of SAM (S-adenosyl methionine) to a final concentration of 0.75 mM. The reaction was carried out at 30° C. and reached maximum after 1-2 hours. The methylated PP2A core enzyme was purified away from LCMT1 by anion exchange chromatography.

### PP2A Phosphatase Assay

The PP2A phosphatase assays were performed as described previously (Chao et al., 2006).

### Example 3

### Crystallographic Data and Refinement for PME-1 and PME-1JPP2A Crystal Structures

The following data was collected and characterized from the crystal of PME-1 and PME-1/PP2A. The atomic coordinates of PME by itself and in complex with PP2A have also been deposited in the Protein Data Bank with the accession codes 3C5V and 3C5W, respectively, each of which is hereby incorporated in its entirety. The data is reproduced in Appendix A and B respectively. Statistics regarding the crystals are presented in Table 1.

TABLE 1
Crystallographic data and refinement.

| Crystal | | | | |
|---|---|---|---|---|
| PME1 | PME1 | PME1 | PME1 | PP2A-PME1 |

Data collection and Phasing

| | | | | | |
|---|---|---|---|---|---|
| Data set | Native | SeMet | SeMet | SeMet | Native |
| | | Peak | Inflection | Remote | |
| Wavelength (Å) | 1.1000 | 0.9791 | 0.9794 | 0.9641 | 1.0809 |
| Resolution (Å) | 50-2.0 | 50-2.6 | 50-2.6 | 50-2.6 | 50-2.7 |
| Outer Shell (Å) | 2.07-2.00 | 2.70-2.60 | 2.70-2.60 | 2.70-2.60 | 2.80-2.70 |
| No. Observations | 106972 | 81583 | 81605 | 81394 | 83552 |
| No. unique | 25077 | 11458 | 11465 | 11460 | 22897 |
| reflections | | | | | |
| Redundancy | 4.3 | 7.1 | 7.1 | 7.1 | 3.6 |
| R-symm¹ | 0.074 (0.293) | 0.123 (0.329) | 0.125 (0.409) | 0.122 (0.359) | 0.111 (0.477) |
| Completeness (%) | 99.5 (97.3) | 99.3 (99.9) | 99.3 (99.9) | 99.3 (100.0) | 98.3 (89.4) |

Phasing

| | | | |
|---|---|---|---|
| Phasing Power | 1.03/1.23 | 0.91/— | 0.68/1.42 |
| (ano/iso) | | | |
| R-cullis (ano/iso) | 0.82/0.58 | 0.85/— | 0.90/0.57 |
| Figure of merit | | | 0.62 |

Refinement

| | | |
|---|---|---|
| Resolution (Å) | 50-2.0 | 50-2.8 |
| No. reflections | 24574 (1191) | 19235 (1018) |
| (free) | | |
| Completeness | 94.9 | 98.6 |
| (%) | | |
| R-factor (%)² | 18.2 | 19.5 |
| R-free (%)² | 21.9 | 26.4 |
| No. atoms | 2562 (283) | 6507 (62) |
| (waters) | | |
| RMSD bond | 0.006 | 0.007 |
| lengths (Å)³ | | |
| RMSD bond | 1.25 | 1.09 |
| angles (°)³ | | |
| RMSD b-factors | 2.9 | 0.43 |
| (Å²)⁴ | | |
| Ramachandran | | |
| plot: | | |
| most favored | 93.3 | 89.3 |
| (%) | | |
| additional (%) | 5.9 | 9.7 |
| generous (%) | 0.4 | 0.6 |
| disallowed (%) | 0.4 | 0.4 |

¹Rsym = ΣhΣi|Ih, i − Ih|/ΣhΣi Ih, i, where Ih is the mean intensity of the i observations of symmetry related reflections of h.
²R = Σ|Fobs − Fcalc|/ΣFobs, where Fobs = FP, and Fcalc is the calculated protein structure factor from the atomic model (Rfree was calculated with 5% of the observed reflections).
³RMSD (root-mean-square deviation) in bond lengths and angles are the deviations from ideal values.
⁴RMSD variation in B-factors across bonded atoms, with the value for PP2A-PME1 being the residual after TLS refinement.

APPENDIX A
COORDINATES OF PP2A-SPECIFIC METHYLESTERASE APO FORM

| HEADER | | HYDROLASE 01-FEB-08 3C5V |
|---|---|---|
| TITLE | | PP2A-SPECIFIC METHYLESTERASE APO FORM (PME) |
| COMPND | | MOL_ID: 1; |
| COMPND | 2 | MOLECULE: PROTEIN PHOSPHATASE METHYLESTERASE 1; |
| COMPND | 3 | CHAIN: A; |
| COMPND | 4 | SYNONYM: PME-1; |
| COMPND | 5 | ENGINEERED: YES |
| SOURCE | | MOL_ID: 1; |
| SOURCE | 2 | ORGANISM_SCIENTIFIC: HOMO SAPIENS; |
| SOURCE | 3 | ORGANISM_COMMON: HUMAN; |
| SOURCE | 4 | GENUS: HOMO; |
| SOURCE | 5 | GENE: PPME1, PME1; |
| SOURCE | 6 | EXPRESSION_SYSTEM: ESCHERICHIA COLI; |
| SOURCE | 7 | EXPRESSION_SYSTEM_GENUS: ESCHERICHIA |
| KEYWDS | | DEMETHYLASE, PP2A, ALTERNATIVE SPLICING, HYDROLASE, |
| KEYWDS | 2 | PHOSPHOPROTEIN, SERINE ESTERASE |
| EXPDTA | | X-RAY DIFFRACTION |
| AUTHOR | | Y. XING, Z. LI, Y. CHEN, J. STOCK, P. D. JEFFREY, Y. SHI |
| REVDAT | 2 | 10-JUN-08 3C5V 1 JRNL |
| REVDAT | 1 | 15-APR-08 3C5V 0 |

| | | |
|---|---|---|
| JRNL | AUTH | Y. XING, Z. LI, Y. CHEN, J. B. STOCK, P. D. JEFFREY, Y. SHI |
| JRNL | TITL | STRUCTURAL MECHANISM OF DEMETHYLATION AND |
| JRNL | TITL 2 | INACTIVATION OF PROTEIN PHOSPHATASE 2A. |
| JRNL | REF | CELL(CAMBRIDGE, MASS.) V. 133 154 2008 |
| JRNL | REFN | ASTM CELLB5 US ISSN 0092-8674 |

| REMARK | 1 | | |
|---|---|---|---|
| REMARK | 2 | | |
| REMARK | 2 | RESOLUTION. 2.00 ANGSTROMS. | |
| REMARK | 3 | | |
| REMARK | 3 | REFINEMENT. | |
| REMARK | 3 | PROGRAM | : CNS 1.2 |
| REMARK | 3 | AUTHORS | : BRUNGER, ADAMS, CLORE, DELANO, GROS, GROSSE- |
| REMARK | 3 | | : KUNSTLEVE, JIANG, KUSZEWSKI, NILGES, PANNU, |
| REMARK | 3 | | : READ, RICE, SIMONSON, WARREN |
| REMARK | 3 | | |

| REMARK | 3 | REFINEMENT TARGET: ENGH & HUBER | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS) | : 2.00 |
| REMARK | 3 | RESOLUTION RANGE LOW | (ANGSTROMS) | : 19.90 |
| REMARK | 3 | DATA CUTOFF | (SIGMA(F)) | : 0.000 |
| REMARK | 3 | DATA CUTOFF HIGH | (ABS(F)) | : 1749029.610 |
| REMARK | 3 | DATA CUTOFF LOW | (ABS(F)) | : 0.0000 |

| | | | | |
|---|---|---|---|---|
| REMARK | 3 | COMPLETENESS (WORKING + TEST) | (%) | : 99.7 |
| REMARK | 3 | NUMBER OF REFLECTIONS | | : 24574 |

| REMARK | 3 | | | |
|---|---|---|---|---|
| REMARK | 3 | FIT TO DATA USED IN REFINEMENT. | | |
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING SET) | : 0.183 |

| REMARK | 3 | FREE R VALUE | | : 0.220 |
|---|---|---|---|---|
| REMARK | 3 | FREE R VALUE TEST SET SIZE | (%) | : 4.800 |

| | | | |
|---|---|---|---|
| REMARK | 3 | FREE R VALUE TEST SET COUNT | : 1191 |
| REMARK | 3 | ESTIMATED ERROR OF FREE R VALUE | : 0.006 |
| REMARK | 3 | | |

| REMARK | 3 | FIT IN THE HIGHEST RESOLUTION BIN. | | |
|---|---|---|---|---|
| REMARK | 3 | TOTAL NUMBER OF BINS USED | | : 6 |
| REMARK | 3 | BIN RESOLUTION RANGE HIGH | (A) | : 2.00 |
| REMARK | 3 | BIN RESOLUTION RANGE LOW | (A) | : 2.13 |
| REMARK | 3 | BIN COMPLETENESS (WORKING + TEST) | (%) | : 99.10 |

| | | | | |
|---|---|---|---|---|
| REMARK | 3 | REFLECTIONS IN BIN | (WORKING SET) | : 3821 |
| REMARK | 3 | BIN R VALUE | (WORKING SET) | : 0.2110 |
| REMARK | 3 | BIN FREE R VALUE | | : 0.2550 |

| | | | | |
|---|---|---|---|---|
| REMARK | 3 | BIN FREE R VALUE TEST SET SIZE | (%) | : 4.90 |

| | | | |
|---|---|---|---|
| REMARK | 3 | BIN FREE R VALUE TEST SET COUNT | : 196 |
| REMARK | 3 | ESTIMATED ERROR OF BIN FREE R VALUE | : 0.018 |
| REMARK | 3 | | |

| REMARK | 3 | NUMBER OF NON-HYDROGEN ATOMS USED IN REFINEMENT. | |
|---|---|---|---|
| REMARK | 3 | PROTEIN ATOMS | : 2278 |
| REMARK | 3 | NUCLEIC ACID ATOMS | : 0 |
| REMARK | 3 | HETEROGEN ATOMS | : 0 |
| REMARK | 3 | SOLVENT ATOMS | : 283 |
| REMARK | 3 | | |
| REMARK | 3 | B VALUES. | |

| | | | | |
|---|---|---|---|---|
| REMARK | 3 | FROM WILSON PLOT | (A**2) | : 16.80 |
| REMARK | 3 | MEAN B VALUE | (OVERALL, A**2) | : 25.10 |

| REMARK | 3 | OVERALL ANISOTROPIC B VALUE. | | |
|---|---|---|---|---|
| REMARK | 3 | B11 (A**2) : −1.43000 | | |
| REMARK | 3 | B22 (A**2) : −1.43000 | | |
| REMARK | 3 | B33 (A**2) : 2.87000 | | |
| REMARK | 3 | B12 (A**2) : 0.00000 | | |
| REMARK | 3 | B13 (A**2) : 0.00000 | | |
| REMARK | 3 | B23 (A**2) : 0.00000 | | |
| REMARK | 3 | | | |
| REMARK | 3 | ESTIMATED COORDINATE ERROR. | | |
| REMARK | 3 | ESD FROM LUZZATI PLOT | (A) | : 0.20 |
| REMARK | 3 | ESD FROM SIGMAA | (A) | : 0.11 |
| REMARK | 3 | LOW RESOLUTION CUTOFF | (A) | : 5.00 |
| REMARK | 3 | | | |
| REMARK | 3 | CROSS-VALIDATED ESTIMATED COORDINATE ERROR. | | |
| REMARK | 3 | ESD FROM C-V LUZZATI PLOT | (A) | : 0.25 |
| REMARK | 3 | ESD FROM C-V SIGMAA | (A) | : 0.17 |
| REMARK | 3 | | | |

| REMARK | 3 | RMS DEVIATIONS FROM IDEAL VALUES. | | |
|---|---|---|---|---|
| REMARK | 3 | BOND LENGTHS | (A) | : 0.007 |
| REMARK | 3 | BOND ANGLES | (DEGREES) | : 1.30 |
| REMARK | 3 | DIHEDRAL ANGLES | (DEGREES) | : 22.70 |
| REMARK | 3 | IMPROPER ANGLES | (DEGREES) | : 0.78 |
| REMARK | 3 | | | |

| REMARK | 3 | ISOTROPIC THERMAL MODEL: RESTRAINED | | | |
|---|---|---|---|---|---|
| REMARK | 3 | | | | |
| REMARK | 3 | ISOTROPIC THERMAL FACTOR RESTRAINTS. | | RMS | SIGMA |
| REMARK | 3 | MAIN-CHAIN BOND | (A**2) | : 2.160 | ; 2.500 |
| REMARK | 3 | MAIN-CHAIN ANGLE | (A**2) | : 3.010 | ; 3.000 |
| REMARK | 3 | SIDE-CHAIN BOND | (A**2) | : 3.500 | ; 3.000 |
| REMARK | 3 | SIDE-CHAIN ANGLE | (A**2) | : 4.880 | ; 3.500 |
| REMARK | 3 | | | | |
| REMARK | 3 | BULK SOLVENT MODELING. | | | |

| | | | |
|---|---|---|---|
| REMARK | 3 | METHOD USED | : FLAT MODEL |
| REMARK | 3 | KSOL | : 0.40 |
| REMARK | 3 | BSOL | : 60.34 |
| REMARK | 3 | | |

| REMARK | 3 | NCS MODEL : NULL | | | |
|---|---|---|---|---|---|
| REMARK | 3 | | | | |
| REMARK | 3 | NCS RESTRAINTS. | | RMS | SIGMA/WEIGHT |
| REMARK | 3 | GROUP 1 POSITIONAL | (A) | : NULL | ; NULL |
| REMARK | 3 | GROUP 1 B-FACTOR | (A**2) | : NULL | ; NULL |
| REMARK | 3 | | | | |

| | | | |
|---|---|---|---|
| REMARK | 3 | PARAMETER FILE 1 | : PROTEIN_REP.PARAM |
| REMARK | 3 | PARAMETER FILE 2 | : DNA-RNA_REP.PARAM |
| REMARK | 3 | PARAMETER FILE 3 | : WATER_REP.PARAM |
| REMARK | 3 | PARAMETER FILE 4 | : ION.PARAM |
| REMARK | 3 | PARAMETER FILE 5 | : NULL |
| REMARK | 3 | TOPOLOGY FILE 1 | : PROTEIN.TOP |
| REMARK | 3 | TOPOLOGY FILE 2 | : NULL |
| REMARK | 3 | TOPOLOGY FILE 3 | : NULL |
| REMARK | 3 | TOPOLOGY FILE 4 | : NULL |
| REMARK | 3 | TOPOLOGY FILE 5 | : NULL |
| REMARK | 3 | | |

| REMARK | 3 | OTHER REFINEMENT REMARKS: BULK SOLVENT MODEL USED | | |
|---|---|---|---|---|
| REMARK | 4 | | | |
| REMARK | 4 | 3C5V COMPLIES WITH FORMAT V. 3.1, 01-AUG-2007 | | |
| REMARK | 100 | | | |
| REMARK | 100 | THIS ENTRY HAS BEEN PROCESSED BY RCSB. | | |
| REMARK | 100 | THE RCSB ID CODE IS RCSB046379. | | |
| REMARK | 200 | | | |
| REMARK | 200 | EXPERIMENTAL DETAILS | | |
| REMARK | 200 | EXPERIMENT TYPE | | : X-RAY DIFFRACTION |
| REMARK | 200 | DATE OF DATA COLLECTION | | : 10-MAR-2005; 18-MAR-2005 |
| REMARK | 200 | TEMPERATURE | (KELVIN) | : 100; 100 |
| REMARK | 200 | PH | | : 7.50 |
| REMARK | 200 | NUMBER OF CRYSTALS USED | | : 1 |
| REMARK | 200 | | | |
| REMARK | 200 | SYNCHROTRON | (Y/N) | : Y; Y |
| REMARK | 200 | RADIATION SOURCE | | : NSLS; NSLS |
| REMARK | 200 | BEAMLINE | | : X25; X25 |

| REMARK | 200 | X-RAY GENERATOR MODEL | | : NULL |
|---|---|---|---|---|
| REMARK | 200 | MONOCHROMATIC OR LAUE | (M/L) | : M; M |
| REMARK | 200 | WAVELENGTH OR RANGE | (A) | : 1.10; 0.97945, 0.97910, |
| REMARK | 200 | | | 0.96410 |
| REMARK | 200 | MONOCHROMATOR | | : SI(111); SI(111) |
| REMARK | 200 | OPTICS | | : NULL |
| REMARK | 200 | | | |
| REMARK | 200 | DETECTOR TYPE | | : CCD; CCD |
| REMARK | 200 | DETECTOR MANUFACTURER | | : ADSC QUANTUM 315; ADSC |
| REMARK | 200 | | | QUANTUM 315 |
| REMARK | 200 | INTENSITY-INTEGRATION SOFTWARE | | : DENZO |
| REMARK | 200 | DATA SCALING SOFTWARE | | : SCALEPACK |
| REMARK | 200 | | | |
| REMARK | 200 | NUMBER OF UNIQUE REFLECTIONS | | : 25077 |
| REMARK | 200 | RESOLUTION RANGE HIGH | (A) | : 2.000 |
| REMARK | 200 | RESOLUTION RANGE LOW | (A) | : 100.000 |

| | | | | |
|---|---|---|---|---|
| REMARK | 200 | REJECTION CRITERIA | (SIGMA(I)) | : −3.000 |
| REMARK | 200 | | | |
| REMARK | 200 | OVERALL. | | |

| | | | | |
|---|---|---|---|---|
| REMARK | 200 | COMPLETENESS FOR RANGE | (%) | : 99.5 |
| REMARK | 200 | DATA REDUNDANCY | | : 4.300 |
| REMARK | 200 | R MERGE | (I) | : NULL |
| REMARK | 200 | R SYM | (I) | : 0.07400 |

| REMARK | 200 | <I/SIGMA(I)> FOR THE DATA SET | | : NULL |
|---|---|---|---|---|
| REMARK | 200 | | | |
| REMARK | 200 | IN THE HIGHEST RESOLUTION SHELL. | | |
| REMARK | 200 | HIGHEST RESOLUTION SHELL, RANGE HIGH (A) : 2.00 | | |
| REMARK | 200 | HIGHEST RESOLUTION SHELL, RANGE LOW (A) : 2.07 | | |
| REMARK | 200 | COMPLETENESS FOR SHELL | (%) | : 97.3 |
| REMARK | 200 | DATA REDUNDANCY IN SHELL | | : NULL |
| REMARK | 200 | R MERGE FOR SHELL | (I) | : 0.29300 |
| REMARK | 200 | R SYM FOR SHELL | (I) | : NULL |
| REMARK | 200 | <I/SIGMA(I)> FOR SHELL | | : NULL |
| REMARK | 200 | | | |

| REMARK | 200 | DIFFRACTION PROTOCOL: SINGLE WAVELENGTH; MAD | |
|---|---|---|---|
| REMARK | 200 | METHOD USED TO DETERMINE THE STRUCTURE: MAD | |
| REMARK | 200 | SOFTWARE USED: SHELXS | |
| REMARK | 200 | STARTING MODEL: NULL | |
| REMARK | 200 | | |
| REMARK | 200 | REMARK: NULL | |
| REMARK | 280 | | |
| REMARK | 280 | CRYSTAL | |
| REMARK | 280 | SOLVENT CONTENT, VS (%): 52.13 | |
| REMARK | 280 | MATTHEWS COEFFICIENT, VM (ANGSTROMS**3/DA): 2.57 | |
| REMARK | 280 | | |
| REMARK | 280 | CRYSTALLIZATION CONDITIONS: 26% V/V JEFFAMINE-2001, 200MM | |
| REMARK | 280 | SODIUM CHLORIDE, 5MM DTT, PH 7.5, VAPOR DIFFUSION, HANGING | |
| REMARK | 280 | DROP, TEMPERATURE 277K | |
| REMARK | 290 | | |
| REMARK | 290 | CRYSTALLOGRAPHIC SYMMETRY | |
| REMARK | 290 | SYMMETRY OPERATORS FOR SPACE GROUP: P 31 2 1 | |
| REMARK | 290 | | |
| REMARK | 290 | SYMOP | SYMMETRY |
| REMARK | 290 | NNNMMM | OPERATOR |
| REMARK | 290 | 1555 | X, Y, Z |
| REMARK | 290 | 2555 | −Y, X − Y, 1/3 + Z |
| REMARK | 290 | 3555 | −X + Y, −X, 2/3 + Z |
| REMARK | 290 | 4555 | Y, X, −Z |
| REMARK | 290 | 5555 | X − Y, −Y, 2/3 − Z |
| REMARK | 290 | 6555 | −X, −X + Y, 1/3 − Z |
| REMARK | 290 | | |

| | | | |
|---|---|---|---|
| REMARK | 290 | WHERE | NNN -> OPERATOR NUMBER |
| REMARK | 290 | | MMM -> TRANSLATION VECTOR |
| REMARK | 290 | | |

| REMARK | 290 | CRYSTALLOGRAPHIC SYMMETRY TRANSFORMATIONS | | | | | |
|---|---|---|---|---|---|---|---|
| REMARK | 290 | THE FOLLOWING TRANSFORMATIONS OPERATE ON THE ATOM/HETATM | | | | | |
| REMARK | 290 | RECORDS IN THIS ENTRY TO PRODUCE CRYSTALLOGRAPHICALLY | | | | | |
| REMARK | 290 | RELATED MOLECULES. | | | | | |
| REMARK | 290 | SMTRY1 | 1 | 1.000000 | 0.000000 | 0.000000 | 0.00000 |
| REMARK | 290 | SMTRY2 | 1 | 0.000000 | 1.000000 | 0.000000 | 0.00000 |
| REMARK | 290 | SMTRY3 | 1 | 0.000000 | 0.000000 | 1.000000 | 0.00000 |
| REMARK | 290 | SMTRY1 | 2 | −0.500000 | −0.866025 | 0.000000 | 0.00000 |
| REMARK | 290 | SMTRY2 | 2 | 0.866025 | −0.500000 | 0.000000 | 0.00000 |
| REMARK | 290 | SMTRY3 | 2 | 0.000000 | 0.000000 | 1.000000 | 30.28100 |
| REMARK | 290 | SMTRY1 | 3 | −0.500000 | 0.866025 | 0.000000 | 0.00000 |
| REMARK | 290 | SMTRY2 | 3 | −0.866025 | −0.500000 | 0.000000 | 0.00000 |
| REMARK | 290 | SMTRY3 | 3 | 0.000000 | 0.000000 | 1.000000 | 60.56200 |
| REMARK | 290 | SMTRY1 | 4 | −0.500000 | 0.866025 | 0.000000 | 0.00000 |
| REMARK | 290 | SMTRY2 | 4 | 0.866025 | 0.500000 | 0.000000 | 0.00000 |
| REMARK | 290 | SMTRY3 | 4 | 0.000000 | 0.000000 | −1.000000 | 0.00000 |
| REMARK | 290 | SMTRY1 | 5 | 1.000000 | 0.000000 | 0.000000 | 0.00000 |
| REMARK | 290 | SMTRY2 | 5 | 0.000000 | −1.000000 | 0.000000 | 0.00000 |
| REMARK | 290 | SMTRY3 | 5 | 0.000000 | 0.000000 | −1.000000 | 60.56200 |
| REMARK | 290 | SMTRY1 | 6 | −0.500000 | −0.866025 | 0.000000 | 0.00000 |
| REMARK | 290 | SMTRY2 | 6 | −0.866025 | 0.500000 | 0.000000 | 0.00000 |
| REMARK | 290 | SMTRY3 | 6 | 0.000000 | 0.000000 | −1.000000 | 30.28100 |
| REMARK | 290 | | | | | | |

| REMARK | 290 | REMARK: NULL | | | | | |
|---|---|---|---|---|---|---|---|
| REMARK | 300 | | | | | | |
| REMARK | 300 | BIOMOLECULE: 1 | | | | | |
| REMARK | 300 | SEE REMARK 350 FOR THE AUTHOR PROVIDED AND/OR PROGRAM | | | | | |
| REMARK | 300 | GENERATED ASSEMBLY INFORMATION FOR THE STRUCTURE IN | | | | | |
| REMARK | 300 | THIS ENTRY. THE REMARK MAY ALSO PROVIDE INFORMATION ON | | | | | |
| REMARK | 300 | BURIED SURFACE AREA. | | | | | |
| REMARK | 350 | | | | | | |
| REMARK | 350 | COORDINATES FOR A COMPLETE MULTIMER REPRESENTING THE KNOWN | | | | | |
| REMARK | 350 | BIOLOGICALLY SIGNIFICANT OLIGOMERIZATION STATE OF THE | | | | | |
| REMARK | 350 | MOLECULE CAN BE GENERATED BY APPLYING BIOMT TRANSFORMATIONS | | | | | |
| REMARK | 350 | GIVEN BELOW. BOTH NON-CRYSTALLOGRAPHIC AND | | | | | |
| REMARK | 350 | CRYSTALLOGRAPHIC OPERATIONS ARE GIVEN. | | | | | |
| REMARK | 350 | | | | | | |
| REMARK | 350 | BIOMOLECULE: 1 | | | | | |
| REMARK | 350 | AUTHOR DETERMINED BIOLOGICAL UNIT: MONOMERIC | | | | | |
| REMARK | 350 | SOFTWARE DETERMINED QUATERNARY STRUCTURE: MONOMERIC | | | | | |
| REMARK | 350 | SOFTWARE USED: PISA | | | | | |
| REMARK | 350 | APPLY THE FOLLOWING TO CHAINS: A | | | | | |
| REMARK | 350 | BIOMT1 | 1 | 1.000000 | 0.000000 | 0.000000 | 0.00000 |
| REMARK | 350 | BIOMT2 | 1 | 0.000000 | 1.000000 | 0.000000 | 0.00000 |
| REMARK | 350 | BIOMT3 | 1 | 0.000000 | 0.000000 | 1.000000 | 0.00000 |
| REMARK | 465 | | | | | | |

| REMARK | 465 | MISSING RESIDUES | | | |
|---|---|---|---|---|---|
| REMARK | 465 | THE FOLLOWING RESIDUES WERE NOT LOCATED IN THE | | | |
| REMARK | 465 | EXPERIMENT. (M = MODEL NUNBER; RES = RESIDUE NAME; C = CHAIN | | | |
| REMARK | 465 | IDENTIFIER; SSEQ = SEQUENCE NUMBER; I = INSERTION CODE.) | | | |
| REMARK | 465 | | | | |
| REMARK | 465 | M RES | | C | SSEQI |
| REMARK | 465 | | GLY | A | 240 |
| REMARK | 465 | | ILE | A | 241 |
| REMARK | 465 | | THR | A | 242 |
| REMARK | 465 | | SER | A | 243 |
| REMARK | 465 | | PRO | A | 244 |
| REMARK | 465 | | GLU | A | 245 |
| REMARK | 465 | | GLY | A | 246 |
| REMARK | 465 | | SER | A | 247 |
| REMARK | 465 | | LYS | A | 248 |
| REMARK | 465 | | LYS | A | 281 |
| REMARK | 465 | | ASP | A | 282 |
| REMARK | 465 | | HIS | A | 283 |
| REMARK | 465 | | GLY | A | 377 |
| REMARK | 465 | | GLY | A | 378 |
| REMARK | 465 | | PHE | A | 379 |
| REMARK | 465 | | GLN | A | 380 |
| REMARK | 465 | | CYS | A | 381 |
| REMARK | 465 | | VAL | A | 382 |
| REMARK | 465 | | PHE | A | 383 |
| REMARK | 465 | | PRO | A | 384 |
| REMARK | 465 | | GLY | A | 385 |
| REMARK | 465 | | CYS | A | 386 |
| REMARK | 500 | | | | |

| REMARK | 500 | GEOMETRY AND STEREOCHEMISTRY | | | | |
|---|---|---|---|---|---|---|
| REMARK | 500 | SUBTOPIC: TORSION ANGLES | | | | |
| REMARK | 500 | | | | | |
| REMARK | 500 | TORSION ANGLES OUTSIDE THE EXPECTED RAMACHANDRAN REGIONS: | | | | |
| REMARK | 500 | (M = MODEL NUMBER; RES = RESIDUE NAME; C = CHAIN IDENTIFIER; | | | | |
| REMARK | 500 | SSEQ = SEQUENCE NUMBER; I = INSERTION CODE). | | | | |
| REMARK | 500 | | | | | |
| REMARK | 500 | STANDARD TABLE: | | | | |
| REMARK | 500 | FORMAT: (10X, I3, 1X, A3, 1X, A1, I4, A1, 4X, F7.2, 3X, F7.2) | | | | |
| REMARK | 500 | | | | | |
| REMARK | 500 | EXPECTED VALUES: GJ KLEYWEGT AND TA JONES (1996). PHI/PSI- | | | | |
| REMARK | 500 | CHOLOGY: RAMACHANDRAN REVISITED. STRUCTURE 4, 1395-1400 | | | | |
| REMARK | 500 | | | | | |
| REMARK | 500 | M | RES | CSSEQI | PSI | PHI |

| | | | | | | |
|---|---|---|---|---|---|---|
| REMARK | 500 | HIS | A | 87 | −157.60 | −100.92 |
| REMARK | 500 | SER | A | 156 | −117.52 | 47.69 |
| REMARK | 500 | ALA | A | 321 | −76.33 | −140.75 |
| REMARK | 500 | HIS | A | 349 | −108.23 | −107.72 |
| REMARK | 500 | | | | | |

| REMARK | 500 | REMARK: NULL | | | | | | | |
|---|---|---|---|---|---|---|---|---|---|
| REMARK | 900 | | | | | | | | |
| REMARK | 900 | RELATED ENTRIES | | | | | | | |
| REMARK | 900 | RELATED ID: 3C5W RELATED DB: PDB | | | | | | | |
| REMARK | 999 | | | | | | | | |
| REMARK | 999 | SEQUENCE | | | | | | | |
| REMARK | 999 | PME-1 WITH INTERNAL PROTEOLYTIC DELETION (DELTA 249-280) | | | | | | | |
| DBREF | 3C5V | A | 39 | 248 | UNP | Q9Y570 | PPME1_HUMAN | 39 | 248 |
| DBREF | 3C5V | A | 281 | 386 | UNP | Q9Y570 | PPME1_HUMAN | 281 | 386 |

| | | | | | | | | | | | | | | | | |
|---|---|---|---|---|---|---|---|---|---|---|---|---|---|---|---|---|
| SEQRES | 1 | A | 316 | ARG | ASP | PHE | SER | PRO | VAL | PRO | TRP | SER | GLN | TYR | PHE | GLU |
| SEQRES | 2 | A | 316 | SER | MET | GLU | ASP | VAL | GLU | VAL | GLU | ASN | GLU | THR | GLY | LYS |
| SEQRES | 3 | A | 316 | ASP | THR | PHE | ARG | VAL | TYR | LYS | SER | GLY | SER | GLU | GLY | PRO |
| SEQRES | 4 | A | 316 | VAL | LEU | LEU | LEU | LEU | HIS | GLY | GLY | HIS | SER | ALA | LEU | |
| SEQRES | 5 | A | 316 | SER | TRP | ALA | VAL | PHE | THR | ALA | ALA | ILE | ILE | SER | ARG | VAL |
| SEQRES | 6 | A | 316 | GLN | CYS | ARG | ILE | VAL | ALA | LEU | ASP | LEU | ARG | SER | HIS | GLY |
| SEQRES | 7 | A | 316 | GLU | THR | LYS | VAL | LYS | ASN | PRO | GLU | ASP | LEU | SER | ALA | GLU |
| SEQRES | 8 | A | 316 | THR | MET | ALA | LYS | ASP | VAL | GLY | ASN | VAL | VAL | GLU | ALA | MET |
| SEQRES | 9 | A | 316 | TYR | GLY | ASP | LEU | PRO | PRO | PRO | ILE | MET | LEU | ILE | GLY | HIS |
| SEQRES | 10 | A | 316 | SER | MET | GLY | GLY | ALA | ILE | ALA | VAL | HIS | THR | ALA | SER | SER |
| SEQRES | 11 | A | 316 | ASN | LEU | VAL | PRO | SER | LEU | LEU | GLY | LEU | CYS | MET | ILE | ASP |
| SEQRES | 12 | A | 316 | VAL | VAL | GLU | GLY | THR | ALA | MET | ASP | ALA | LEU | ASN | SER | MET |
| SEQRES | 13 | A | 316 | GLN | ASN | PHE | LEU | ARG | GLY | ARG | PRO | LYS | THR | PHE | LYS | SER |
| SEQRES | 14 | A | 316 | LEU | GLU | ASN | ALA | ILE | GLU | TRP | SER | VAL | LYS | SER | GLY | GLN |
| SEQRES | 15 | A | 316 | ILE | ARG | ASN | LEU | GLU | SER | ALA | ARG | VAL | SER | MET | VAL | GLY |
| SEQRES | 16 | A | 316 | GLN | VAL | LYS | GLN | CYS | GLU | GLY | ILE | THR | SER | PRO | GLU | GLY |
| SEQRES | 17 | A | 316 | SER | LYS | LYS | ASP | HIS | PRO | TYR | THR | TRP | ARG | ILE | GLU | LEU |
| SEQRES | 18 | A | 316 | ALA | LYS | THR | GLU | LYS | TYR | TRP | ASP | GLY | TRP | PHE | ARG | GLY |
| SEQRES | 19 | A | 316 | LEU | SER | ASN | LEU | PHE | LEU | SER | CYS | PRO | ILE | PRO | LYS | LEU |
| SEQRES | 20 | A | 316 | LEU | LEU | LEU | ALA | GLY | VAL | ASP | ARG | LEU | ASP | LYS | ASP | LEU |
| SEQRES | 21 | A | 316 | THR | ILE | GLY | GLN | MET | GLN | GLY | LYS | PHE | GLN | MET | GLN | VAL |
| SEQRES | 22 | A | 316 | LEU | PRO | GLN | CYS | GLY | HIS | ALA | VAL | HIS | GLU | ASP | ALA | PRO |
| SEQRES | 23 | A | 316 | ASP | LYS | VAL | ALA | GLU | ALA | VAL | ALA | THR | PHE | LEU | ILE | ARG |
| SEQRES | 24 | A | 316 | HIS | ARG | PHE | ALA | GLU | PRO | ILE | GLY | GLY | PHE | GLN | CYS | VAL |
| SEQRES | 25 | A | 316 | PHE | PRO | GLY | CYS | | | | | | | | | |

| | | | |
|---|---|---|---|
| FORMUL | 2 | HOH | *283(H2O) |

| | | | | | | | | | | |
|---|---|---|---|---|---|---|---|---|---|---|
| HELIX | 1 | 1 | PRO | A | 45 | TYR | A | 49 | 5 | 5 |
| HELIX | 2 | 2 | SER | A | 88 | SER | A | 91 | 5 | 4 |
| HELIX | 3 | 3 | TRP | A | 92 | SER | A | 101 | 1 | 10 |
| HELIX | 4 | 4 | SER | A | 127 | GLY | A | 144 | 1 | 18 |
| HELIX | 5 | 5 | SER | A | 156 | SER | A | 168 | 1 | 13 |
| HELIX | 6 | 6 | VAL | A | 183 | ARG | A | 201 | 1 | 19 |
| HELIX | 7 | 7 | SER | A | 207 | SER | A | 218 | 1 | 12 |
| HELIX | 8 | 8 | ASN | A | 223 | GLN | A | 234 | 1 | 12 |
| HELIX | 9 | 9 | GLU | A | 290 | LYS | A | 293 | 5 | 4 |
| HELIX | 10 | 10 | THR | A | 294 | ARG | A | 303 | 1 | 10 |
| HELIX | 11 | 11 | GLY | A | 304 | CYS | A | 312 | 1 | 9 |
| HELIX | 12 | 12 | ASP | A | 327 | GLN | A | 336 | 1 | 10 |
| HELIX | 13 | 13 | ALA | A | 350 | ALA | A | 355 | 1 | 6 |
| HELIX | 14 | 14 | ALA | A | 355 | HIS | A | 370 | 1 | 16 |

| | | | | | | | | | | | | | | | | | | |
|---|---|---|---|---|---|---|---|---|---|---|---|---|---|---|---|---|---|---|
| SHEET | 1 | A | 8 | SER | A | 52 | ASN | A | 60 | 0 | | | | | | | | |
| SHEET | 2 | A | 8 | GLY | A | 63 | SER | A | 72 | −1 | O | ASP | A | 65 | N | VAL | A | 58 |
| SHEET | 3 | A | 8 | ARG | A | 106 | LEU | A | 110 | −1 | O | ALA | A | 109 | N | TYR | A | 70 |
| SHEET | 4 | A | 8 | VAL | A | 78 | LEU | A | 82 | 1 | N | LEU | A | 79 | O | ARG | A | 106 |
| SHEET | 5 | A | 8 | ILE | A | 150 | HIS | A | 155 | 1 | O | ILE | A | 153 | N | LEU | A | 82 |
| SHEET | 6 | A | 8 | LEU | A | 174 | ILE | A | 180 | 1 | O | LEU | A | 175 | N | ILE | A | 150 |
| SHEET | 7 | A | 8 | LYS | A | 316 | LEU | A | 320 | 1 | O | LEU | A | 317 | N | LEU | A | 177 |
| SHEET | 8 | A | 8 | GLN | A | 340 | VAL | A | 343 | 1 | O | GLN | A | 340 | N | LYS | A | 316 |
| SHEET | 1 | B | 2 | VAL | A | 235 | GLN | A | 237 | 0 | | | | | | | | |
| SHEET | 2 | B | 2 | TYR | A | 285 | TRP | A | 287 | −1 | O | THR | A | 286 | N | LYS | A | 236 |

| | | | | | | | | |
|---|---|---|---|---|---|---|---|---|
| CRYST1 | 82.512 | 82.512 | 90.843 | 90.00 | 90.00 | 120.00 | P 31 2 1 | 6 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.012119 | 0.006997 | 0.000000 | 0.00000 |
| SCALE2 | 0.000000 | 0.013994 | 0.000000 | 0.00000 |
| SCALE3 | 0.000000 | 0.000000 | 0.011008 | 0.00000 |

| | | | | | | | | | | | |
|---|---|---|---|---|---|---|---|---|---|---|---|
| ATOM | 1 | N | ARG | A | 39 | 36.930 | −18.509 | −15.684 | 1.00 | 33.68 | N |
| ATOM | 2 | CA | ARG | A | 39 | 36.640 | −17.391 | −14.740 | 1.00 | 29.93 | C |
| ATOM | 3 | C | ARG | A | 39 | 37.399 | −16.138 | −15.158 | 1.00 | 27.15 | C |
| ATOM | 4 | O | ARG | A | 39 | 38.383 | −16.220 | −15.892 | 1.00 | 26.98 | O |
| ATOM | 5 | CB | ARG | A | 39 | 37.033 | −17.786 | −13.309 | 1.00 | 32.28 | C |
| ATOM | 6 | CG | ARG | A | 39 | 36.291 | −19.008 | −12.789 | 1.00 | 36.94 | C |
| ATOM | 7 | CD | ARG | A | 39 | 36.341 | −19.126 | −11.267 | 1.00 | 39.34 | C |
| ATOM | 8 | NE | ARG | A | 39 | 37.657 | −19.464 | −10.725 | 1.00 | 45.43 | N |
| ATOM | 9 | CZ | ARG | A | 39 | 38.353 | −20.555 | −11.041 | 1.00 | 48.80 | C |
| ATOM | 10 | NH1 | ARG | A | 39 | 37.871 | −21.433 | −11.912 | 1.00 | 52.17 | N |
| ATOM | 11 | NH2 | ARG | A | 39 | 39.528 | −20.781 | −10.465 | 1.00 | 46.98 | N |
| ATOM | 12 | N | ASP | A | 40 | 36.935 | −14.981 | −14.689 | 1.00 | 25.30 | N |
| ATOM | 13 | CA | ASP | A | 40 | 37.568 | −13.700 | −15.008 | 1.00 | 22.36 | C |
| ATOM | 14 | C | ASP | A | 40 | 38.526 | −13.291 | −13.883 | 1.00 | 23.44 | C |
| ATOM | 15 | O | ASP | A | 40 | 38.098 | −13.044 | −12.755 | 1.00 | 20.86 | O |
| ATOM | 16 | CB | ASP | A | 40 | 36.492 | −12.627 | −15.185 | 1.00 | 26.23 | C |
| ATOM | 17 | CG | ASP | A | 40 | 37.032 | −11.345 | −15.781 | 1.00 | 26.49 | C |
| ATOM | 18 | OD1 | ASP | A | 40 | 38.266 | −11.153 | −15.803 | 1.00 | 25.14 | O |
| ATOM | 19 | OD2 | ASP | A | 40 | 36.207 | −10.521 | −16.223 | 1.00 | 33.06 | O |
| ATOM | 20 | N | PHE | A | 41 | 39.815 | −13.202 | −14.207 | 1.00 | 21.41 | N |
| ATOM | 21 | CA | PHE | A | 41 | 40.860 | −12.849 | −13.241 | 1.00 | 22.56 | C |
| ATOM | 22 | C | PHE | A | 41 | 41.450 | −11.452 | −13.438 | 1.00 | 20.13 | C |
| ATOM | 23 | O | PHE | A | 41 | 42.469 | −11.118 | −12.831 | 1.00 | 22.05 | O |
| ATOM | 24 | CB | PHE | A | 41 | 41.997 | −13.879 | −13.315 | 1.00 | 20.82 | C |
| ATOM | 25 | CG | PHE | A | 41 | 41.579 | −15.272 | −12.945 | 1.00 | 23.74 | C |
| ATOM | 26 | CD1 | PHE | A | 41 | 41.428 | −15.633 | −11.613 | 1.00 | 20.61 | C |
| ATOM | 27 | CD2 | PHE | A | 41 | 41.327 | −16.224 | −13.932 | 1.00 | 22.16 | C |
| ATOM | 28 | CE1 | PHE | A | 41 | 41.035 | −16.925 | −11.267 | 1.00 | 23.50 | C |
| ATOM | 29 | CE2 | PHE | A | 41 | 40.932 | −17.517 | −13.599 | 1.00 | 23.54 | C |
| ATOM | 30 | CZ | PHE | A | 41 | 40.787 | −17.870 | −12.266 | 1.00 | 23.87 | C |
| ATOM | 31 | N | SER | A | 42 | 40.820 | −10.647 | −14.290 | 1.00 | 20.37 | N |
| ATOM | 32 | CA | SER | A | 42 | 41.295 | −9.295 | −14.558 | 1.00 | 21.13 | C |
| ATOM | 33 | C | SER | A | 42 | 41.187 | −8.433 | −13.302 | 1.00 | 19.48 | C |
| ATOM | 34 | O | SER | A | 42 | 40.303 | −8.642 | −12.468 | 1.00 | 22.49 | O |
| ATOM | 35 | CB | SER | A | 42 | 40.493 | −8.662 | −15.704 | 1.00 | 20.81 | C |
| ATOM | 36 | OG | SER | A | 42 | 39.108 | −8.625 | −15.404 | 1.00 | 23.36 | O |
| ATOM | 37 | N | PRO | A | 43 | 42.094 | −7.459 | −13.153 | 1.00 | 18.84 | N |
| ATOM | 38 | CA | PRO | A | 43 | 42.151 | −6.538 | −12.014 | 1.00 | 20.04 | C |
| ATOM | 39 | C | PRO | A | 43 | 40.904 | −5.673 | −11.871 | 1.00 | 21.88 | C |
| ATOM | 40 | O | PRO | A | 43 | 40.330 | −5.227 | −12.864 | 1.00 | 20.75 | O |
| ATOM | 41 | CB | PRO | A | 43 | 43.372 | −5.679 | −12.319 | 1.00 | 22.03 | C |
| ATOM | 42 | CG | PRO | A | 43 | 44.178 | −6.511 | −13.266 | 1.00 | 20.85 | C |
| ATOM | 43 | CD | PRO | A | 43 | 43.146 | −7.133 | −14.129 | 1.00 | 21.83 | C |
| ATOM | 44 | N | VAL | A | 44 | 40.502 | −5.445 | −10.627 | 1.00 | 21.12 | N |
| ATOM | 45 | CA | VAL | A | 44 | 39.353 | −4.608 | −10.306 | 1.00 | 22.89 | C |
| ATOM | 46 | C | VAL | A | 44 | 39.916 | −3.464 | −9.463 | 1.00 | 23.62 | C |
| ATOM | 47 | O | VAL | A | 44 | 40.629 | −3.699 | −8.486 | 1.00 | 21.21 | O |
| ATOM | 48 | CB | VAL | A | 44 | 38.302 | −5.393 | −9.498 | 1.00 | 23.75 | C |
| ATOM | 49 | CG1 | VAL | A | 44 | 37.220 | −4.441 | −8.972 | 1.00 | 24.01 | C |
| ATOM | 50 | CG2 | VAL | A | 44 | 37.676 | −6.473 | −10.379 | 1.00 | 17.83 | C |
| ATOM | 51 | N | PRO | A | 45 | 39.614 | −2.208 | −9.836 | 1.00 | 23.17 | N |
| ATOM | 52 | CA | PRO | A | 45 | 40.111 | −1.040 | −9.102 | 1.00 | 21.35 | C |
| ATOM | 53 | C | PRO | A | 45 | 39.443 | −0.751 | −7.755 | 1.00 | 17.32 | C |
| ATOM | 54 | O | PRO | A | 45 | 38.308 | −1.147 | −7.505 | 1.00 | 17.26 | O |
| ATOM | 55 | CB | PRO | A | 45 | 39.916 | 0.094 | −10.106 | 1.00 | 23.95 | C |
| ATOM | 56 | CG | PRO | A | 45 | 38.658 | −0.299 | −10.780 | 1.00 | 25.71 | C |
| ATOM | 57 | CD | PRO | A | 45 | 38.826 | −1.791 | −11.010 | 1.00 | 25.99 | C |
| ATOM | 58 | N | TRP | A | 46 | 40.166 | −0.035 | −6.902 | 1.00 | 21.73 | N |
| ATOM | 59 | CA | TRP | A | 46 | 39.679 | 0.325 | −5.579 | 1.00 | 20.36 | C |
| ATOM | 60 | C | TRP | A | 46 | 38.336 | 1.047 | −5.616 | 1.00 | 21.07 | C |
| ATOM | 61 | O | TRP | A | 46 | 37.527 | 0.915 | −4.700 | 1.00 | 19.23 | O |
| ATOM | 62 | CB | TRP | A | 46 | 40.696 | 1.223 | −4.867 | 1.00 | 19.64 | C |
| ATOM | 63 | CG | TRP | A | 46 | 40.938 | 2.551 | −5.543 | 1.00 | 23.61 | C |
| ATOM | 64 | CD1 | TRP | A | 46 | 41.937 | 2.854 | −6.425 | 1.00 | 25.25 | C |
| ATOM | 65 | CD2 | TRP | A | 46 | 40.159 | 3.748 | −5.393 | 1.00 | 22.71 | C |
| ATOM | 66 | NE1 | TRP | A | 46 | 41.830 | 4.165 | −6.832 | 1.00 | 26.40 | N |
| ATOM | 67 | CE2 | TRP | A | 46 | 40.747 | 4.735 | −6.215 | 1.00 | 24.55 | C |
| ATOM | 68 | CE3 | TRP | A | 46 | 39.020 | 4.081 | −4.644 | 1.00 | 22.53 | C |
| ATOM | 69 | CZ2 | TRP | A | 46 | 40.234 | 6.039 | −6.310 | 1.00 | 25.27 | C |
| ATOM | 70 | CZ3 | TRP | A | 46 | 38.509 | 5.379 | −4.739 | 1.00 | 22.61 | C |
| ATOM | 71 | CH2 | TRP | A | 46 | 39.120 | 6.339 | −5.567 | 1.00 | 22.52 | C |
| ATOM | 72 | N | SER | A | 47 | 38.101 | 1.803 | −6.682 | 1.00 | 20.14 | N |
| ATOM | 73 | CA | SER | A | 47 | 36.875 | 2.581 | −6.813 | 1.00 | 20.31 | C |
| ATOM | 74 | C | SER | A | 47 | 35.591 | 1.774 | −6.921 | 1.00 | 22.48 | C |
| ATOM | 75 | O | SER | A | 47 | 34.497 | 2.335 | −6.826 | 1.00 | 21.22 | O |
| ATOM | 76 | CB | SER | A | 47 | 36.995 | 3.544 | −7.998 | 1.00 | 21.19 | C |
| ATOM | 77 | OG | SER | A | 47 | 37.363 | 2.849 | −9.166 | 1.00 | 22.04 | O |
| ATOM | 78 | N | GLN | A | 48 | 35.706 | 0.466 | −7.128 | 1.00 | 19.87 | N |
| ATOM | 79 | CA | GLN | A | 48 | 34.513 | −0.369 | −7.202 | 1.00 | 18.97 | C |
| ATOM | 80 | C | GLN | A | 48 | 34.042 | −0.665 | −5.781 | 1.00 | 20.14 | C |
| ATOM | 81 | O | GLN | A | 48 | 32.922 | −1.140 | −5.576 | 1.00 | 18.58 | O |
| ATOM | 82 | CB | GLN | A | 48 | 34.809 | −1.700 | −7.901 | 1.00 | 23.36 | C |
| ATOM | 83 | CG | GLN | A | 48 | 34.856 | −1.643 | −9.426 | 1.00 | 30.89 | C |
| ATOM | 84 | CD | GLN | A | 48 | 33.587 | −1.059 | −10.024 | 1.00 | 32.29 | C |
| ATOM | 85 | OE1 | GLN | A | 48 | 33.492 | 0.148 | −10.248 | 1.00 | 34.34 | O |
| ATOM | 86 | NE2 | GLN | A | 48 | 32.600 | −1.912 | −10.269 | 1.00 | 29.56 | N |
| ATOM | 87 | N | TYR | A | 49 | 34.898 | −0.375 | −4.802 | 1.00 | 19.02 | N |
| ATOM | 88 | CA | TYR | A | 49 | 34.577 | −0.659 | −3.406 | 1.00 | 17.48 | C |
| ATOM | 89 | C | TYR | A | 49 | 34.639 | 0.519 | −2.444 | 1.00 | 19.58 | C |
| ATOM | 90 | O | TYR | A | 49 | 33.851 | 0.585 | −1.500 | 1.00 | 23.32 | O |
| ATOM | 91 | CB | TYR | A | 49 | 35.489 | −1.779 | −2.907 | 1.00 | 20.66 | C |
| ATOM | 92 | CG | TYR | A | 49 | 35.213 | −3.093 | −3.594 | 1.00 | 19.12 | C |
| ATOM | 93 | CD1 | TYR | A | 49 | 34.159 | −3.908 | −3.179 | 1.00 | 19.24 | C |
| ATOM | 94 | CD2 | TYR | A | 49 | 35.966 | −3.494 | −4.699 | 1.00 | 20.83 | C |
| ATOM | 95 | CE1 | TYR | A | 49 | 33.856 | −5.091 | −3.850 | 1.00 | 19.50 | C |
| ATOM | 96 | CE2 | TYR | A | 49 | 35.673 | −4.679 | −5.379 | 1.00 | 21.27 | C |
| ATOM | 97 | CZ | TYR | A | 49 | 34.618 | −5.470 | −4.951 | 1.00 | 18.76 | C |
| ATOM | 98 | OH | TYR | A | 49 | 34.309 | −6.633 | −5.625 | 1.00 | 20.53 | O |
| ATOM | 99 | N | PHE | A | 50 | 35.576 | 1.436 | −2.670 | 1.00 | 17.55 | N |
| ATOM | 100 | CA | PHE | A | 50 | 35.732 | 2.608 | −1.807 | 1.00 | 20.78 | C |
| ATOM | 101 | C | PHE | A | 50 | 35.252 | 3.861 | −2.537 | 1.00 | 21.98 | C |
| ATOM | 102 | O | PHE | A | 50 | 35.425 | 3.981 | −3.749 | 1.00 | 20.64 | O |
| ATOM | 103 | CB | PHE | A | 50 | 37.204 | 2.791 | −1.422 | 1.00 | 19.08 | C |
| ATOM | 104 | CG | PHE | A | 50 | 37.777 | 1.646 | −0.620 | 1.00 | 21.16 | C |
| ATOM | 105 | CD1 | PHE | A | 50 | 37.538 | 1.546 | 0.745 | 1.00 | 20.35 | C |
| ATOM | 106 | CD2 | PHE | A | 50 | 38.573 | 0.685 | −1.229 | 1.00 | 21.24 | C |
| ATOM | 107 | CE1 | PHE | A | 50 | 38.087 | 0.508 | 1.495 | 1.00 | 16.95 | C |
| ATOM | 108 | CE2 | PHE | A | 50 | 39.128 | −0.359 | −0.490 | 1.00 | 17.59 | C |
| ATOM | 109 | CZ | PHE | A | 50 | 38.885 | −0.445 | 0.875 | 1.00 | 18.83 | C |
| ATOM | 110 | N | GLU | A | 51 | 34.661 | 4.796 | −1.800 | 1.00 | 23.58 | N |
| ATOM | 111 | CA | GLU | A | 51 | 34.165 | 6.035 | −2.396 | 1.00 | 23.08 | C |
| ATOM | 112 | C | GLU | A | 51 | 35.291 | 6.986 | −2.777 | 1.00 | 20.93 | C |
| ATOM | 113 | O | GLU | A | 51 | 35.176 | 7.738 | −3.745 | 1.00 | 23.51 | O |
| ATOM | 114 | CB | GLU | A | 51 | 33.202 | 6.735 | −1.435 | 1.00 | 24.18 | C |
| ATOM | 115 | CG | GLU | A | 51 | 31.823 | 6.105 | −1.390 | 1.00 | 32.18 | C |
| ATOM | 116 | CD | GLU | A | 51 | 30.997 | 6.589 | −0.211 | 1.00 | 36.69 | C |
| ATOM | 117 | OE1 | GLU | A | 51 | 31.040 | 7.797 | 0.089 | 1.00 | 38.40 | O |
| ATOM | 118 | OE2 | GLU | A | 51 | 30.298 | 5.763 | 0.411 | 1.00 | 41.29 | O |
| ATOM | 119 | N | SER | A | 52 | 36.382 | 6.955 | −2.025 | 1.00 | 20.19 | N |
| ATOM | 120 | CA | SER | A | 52 | 37.507 | 7.832 | −2.324 | 1.00 | 19.57 | C |
| ATOM | 121 | C | SER | A | 52 | 38.801 | 7.279 | −1.765 | 1.00 | 21.80 | C |
| ATOM | 122 | O | SER | A | 52 | 38.803 | 6.308 | −1.011 | 1.00 | 20.53 | O |
| ATOM | 123 | CB | SER | A | 52 | 37.270 | 9.221 | −1.731 | 1.00 | 22.60 | C |
| ATOM | 124 | OG | SER | A | 52 | 37.258 | 9.168 | −0.312 | 1.00 | 25.39 | O |
| ATOM | 125 | N | MET | A | 53 | 39.904 | 7.910 | −2.141 | 1.00 | 19.67 | N |
| ATOM | 126 | CA | MET | A | 53 | 41.210 | 7.505 | −1.658 | 1.00 | 24.12 | C |
| ATOM | 127 | C | MET | A | 53 | 42.022 | 8.776 | −1.475 | 1.00 | 24.37 | C |
| ATOM | 128 | O | MET | A | 53 | 41.755 | 9.787 | −2.119 | 1.00 | 23.06 | O |
| ATOM | 129 | CB | MET | A | 53 | 41.894 | 6.590 | −2.667 | 1.00 | 26.76 | C |
| ATOM | 130 | CG | MET | A | 53 | 42.326 | 7.299 | −3.917 | 1.00 | 31.35 | C |
| ATOM | 131 | SD | MET | A | 53 | 44.070 | 7.025 | −4.195 | 1.00 | 43.65 | S |
| ATOM | 132 | CE | MET | A | 53 | 44.754 | 8.218 | −3.146 | 1.00 | 22.42 | C |
| ATOM | 133 | N | GLU | A | 54 | 43.013 | 8.727 | −0.597 | 1.00 | 23.48 | N |
| ATOM | 134 | CA | GLU | A | 54 | 43.830 | 9.899 | −0.348 | 1.00 | 26.36 | C |
| ATOM | 135 | C | GLU | A | 54 | 45.236 | 9.485 | 0.044 | 1.00 | 26.22 | C |
| ATOM | 136 | O | GLU | A | 54 | 45.462 | 8.386 | 0.549 | 1.00 | 20.75 | O |
| ATOM | 137 | CB | GLU | A | 54 | 43.212 | 10.725 | 0.788 | 1.00 | 30.48 | C |
| ATOM | 138 | CG | GLU | A | 54 | 43.218 | 12.229 | 0.566 | 1.00 | 41.31 | C |
| ATOM | 139 | CD | GLU | A | 54 | 42.316 | 12.669 | −0.581 | 1.00 | 43.97 | C |
| ATOM | 140 | OE1 | GLU | A | 54 | 41.084 | 12.445 | −0.508 | 1.00 | 46.24 | O |
| ATOM | 141 | OE2 | GLU | A | 54 | 42.842 | 13.242 | −1.560 | 1.00 | 48.79 | O |
| ATOM | 142 | N | ASP | A | 55 | 46.188 | 10.363 | −0.223 | 1.00 | 23.19 | N |
| ATOM | 143 | CA | ASP | A | 55 | 47.559 | 10.122 | 0.163 | 1.00 | 24.59 | C |
| ATOM | 144 | C | ASP | A | 55 | 47.766 | 11.146 | 1.272 | 1.00 | 24.99 | C |
| ATOM | 145 | O | ASP | A | 55 | 47.902 | 12.339 | 1.016 | 1.00 | 23.61 | O |
| ATOM | 146 | CB | ASP | A | 55 | 48.500 | 10.346 | −1.024 | 1.00 | 28.10 | C |
| ATOM | 147 | CG | ASP | A | 55 | 48.491 | 9.169 | −1.993 | 1.00 | 31.19 | C |
| ATOM | 148 | OD1 | ASP | A | 55 | 48.698 | 8.024 | −1.537 | 1.00 | 28.71 | O |
| ATOM | 149 | OD2 | ASP | A | 55 | 48.276 | 9.378 | −3.203 | 1.00 | 34.84 | O |
| ATOM | 150 | N | VAL | A | 56 | 47.740 | 10.670 | 2.512 | 1.00 | 22.37 | N |
| ATOM | 151 | CA | VAL | A | 56 | 47.875 | 11.534 | 3.676 | 1.00 | 19.40 | C |
| ATOM | 152 | C | VAL | A | 56 | 49.320 | 11.889 | 4.003 | 1.00 | 22.05 | C |
| ATOM | 153 | O | VAL | A | 56 | 50.143 | 11.020 | 4.286 | 1.00 | 17.46 | O |
| ATOM | 154 | CB | VAL | A | 56 | 47.210 | 10.874 | 4.906 | 1.00 | 19.69 | C |
| ATOM | 155 | CG1 | VAL | A | 56 | 47.265 | 11.803 | 6.100 | 1.00 | 18.07 | C |
| ATOM | 156 | CG2 | VAL | A | 56 | 45.764 | 10.513 | 4.571 | 1.00 | 18.83 | C |
| ATOM | 157 | N | GLU | A | 57 | 49.620 | 13.183 | 3.973 | 1.00 | 20.44 | N |
| ATOM | 158 | CA | GLU | A | 57 | 50.962 | 13.648 | 4.259 | 1.00 | 23.32 | C |
| ATOM | 159 | C | GLU | A | 57 | 51.214 | 13.710 | 5.753 | 1.00 | 23.64 | C |
| ATOM | 160 | O | GLU | A | 57 | 50.382 | 14.208 | 6.511 | 1.00 | 21.70 | O |
| ATOM | 161 | CB | GLU | A | 57 | 51.182 | 15.036 | 3.654 | 1.00 | 29.72 | C |
| ATOM | 162 | CG | GLU | A | 57 | 51.239 | 15.072 | 2.142 | 1.00 | 39.00 | C |
| ATOM | 163 | CD | GLU | A | 57 | 51.476 | 16.477 | 1.614 | 1.00 | 45.70 | C |
| ATOM | 164 | OE1 | GLU | A | 57 | 50.568 | 17.327 | 1.742 | 1.00 | 48.76 | O |
| ATOM | 165 | OE2 | GLU | A | 57 | 52.577 | 16.736 | 1.083 | 1.00 | 49.39 | O |
| ATOM | 166 | N | VAL | A | 58 | 52.365 | 13.197 | 6.170 | 1.00 | 21.75 | N |
| ATOM | 167 | CA | VAL | A | 58 | 52.750 | 13.213 | 7.573 | 1.00 | 25.41 | C |
| ATOM | 168 | C | VAL | A | 58 | 54.212 | 13.625 | 7.658 | 1.00 | 31.04 | C |
| ATOM | 169 | O | VAL | A | 58 | 55.064 | 13.070 | 6.966 | 1.00 | 30.03 | O |
| ATOM | 170 | CB | VAL | A | 58 | 52.563 | 11.819 | 8.230 | 1.00 | 24.66 | C |
| ATOM | 171 | CG1 | VAL | A | 58 | 53.158 | 11.811 | 9.627 | 1.00 | 26.67 | C |
| ATOM | 172 | CG2 | VAL | A | 58 | 51.080 | 11.477 | 8.299 | 1.00 | 21.86 | C |
| ATOM | 173 | N | GLU | A | 59 | 54.497 | 14.619 | 8.490 | 1.00 | 39.82 | N |
| ATOM | 174 | CA | GLU | A | 59 | 55.864 | 15.089 | 8.665 | 1.00 | 51.34 | C |
| ATOM | 175 | C | GLU | A | 59 | 56.350 | 14.556 | 10.005 | 1.00 | 56.01 | C |
| ATOM | 176 | O | GLU | A | 59 | 55.939 | 15.035 | 11.061 | 1.00 | 59.42 | O |
| ATOM | 177 | CB | GLU | A | 59 | 55.909 | 16.620 | 8.660 | 1.00 | 53.10 | C |
| ATOM | 178 | CG | GLU | A | 59 | 55.071 | 17.261 | 9.754 | 1.00 | 62.22 | C |
| ATOM | 179 | CD | GLU | A | 59 | 55.238 | 18.766 | 9.822 | 1.00 | 67.40 | C |
| ATOM | 180 | OE1 | GLU | A | 59 | 56.393 | 19.233 | 9.938 | 1.00 | 70.37 | O |
| ATOM | 181 | OE2 | GLU | A | 59 | 54.213 | 19.481 | 9.769 | 1.00 | 69.86 | O |
| ATOM | 182 | N | ASN | A | 60 | 57.215 | 13.551 | 9.966 | 1.00 | 61.02 | N |
| ATOM | 183 | CA | ASN | A | 60 | 57.724 | 12.975 | 11.199 | 1.00 | 66.15 | C |
| ATOM | 184 | C | ASN | A | 60 | 59.226 | 13.166 | 11.385 | 1.00 | 69.12 | C |
| ATOM | 185 | O | ASN | A | 60 | 59.895 | 13.805 | 10.568 | 1.00 | 71.01 | O |
| ATOM | 186 | CB | ASN | A | 60 | 57.362 | 11.486 | 11.273 | 1.00 | 67.86 | C |
| ATOM | 187 | CG | ASN | A | 60 | 57.777 | 10.717 | 10.033 | 1.00 | 69.50 | C |
| ATOM | 188 | OD1 | ASN | A | 60 | 57.471 | 9.532 | 9.897 | 1.00 | 70.34 | O |
| ATOM | 189 | ND2 | ASN | A | 60 | 58.478 | 11.385 | 9.122 | 1.00 | 71.26 | N |
| ATOM | 190 | N | GLU | A | 61 | 59.737 | 12.605 | 12.477 | 1.00 | 71.01 | N |
| ATOM | 191 | CA | GLU | A | 61 | 61.148 | 12.694 | 12.835 | 1.00 | 72.72 | C |
| ATOM | 192 | C | GLU | A | 61 | 62.111 | 12.414 | 11.682 | 1.00 | 71.45 | C |
| ATOM | 193 | O | GLU | A | 61 | 63.182 | 13.019 | 11.607 | 1.00 | 71.13 | O |
| ATOM | 194 | CB | GLU | A | 61 | 61.434 | 11.734 | 13.992 | 1.00 | 74.95 | C |
| ATOM | 195 | CG | GLU | A | 61 | 60.508 | 11.937 | 15.182 | 1.00 | 79.18 | C |
| ATOM | 196 | CD | GLU | A | 61 | 60.677 | 10.873 | 16.247 | 1.00 | 81.59 | C |
| ATOM | 197 | OE1 | GLU | A | 61 | 59.937 | 10.918 | 17.253 | 1.00 | 83.73 | O |
| ATOM | 198 | OE2 | GLU | A | 61 | 61.549 | 9.994 | 16.081 | 1.00 | 83.17 | O |
| ATOM | 199 | N | THR | A | 62 | 61.734 | 11.504 | 10.787 | 1.00 | 68.87 | N |
| ATOM | 200 | CA | THR | A | 62 | 62.589 | 11.162 | 9.654 | 1.00 | 66.12 | C |
| ATOM | 201 | C | THR | A | 62 | 62.289 | 11.986 | 8.405 | 1.00 | 63.79 | C |
| ATOM | 202 | O | THR | A | 62 | 62.886 | 11.762 | 7.351 | 1.00 | 63.65 | O |
| ATOM | 203 | CB | THR | A | 62 | 62.480 | 9.658 | 9.291 | 1.00 | 66.23 | C |
| ATOM | 204 | OG1 | THR | A | 62 | 61.128 | 9.339 | 8.940 | 1.00 | 66.65 | O |
| ATOM | 205 | CG2 | THR | A | 62 | 62.921 | 8.790 | 10.461 | 1.00 | 66.32 | C |
| ATOM | 206 | N | GLY | A | 63 | 61.369 | 12.940 | 8.519 | 1.00 | 60.91 | N |
| ATOM | 207 | CA | GLY | A | 63 | 61.041 | 13.771 | 7.375 | 1.00 | 56.45 | C |
| ATOM | 208 | C | GLY | A | 63 | 59.562 | 13.861 | 7.050 | 1.00 | 54.28 | C |
| ATOM | 209 | O | GLY | A | 63 | 58.724 | 13.987 | 7.945 | 1.00 | 54.17 | O |
| ATOM | 210 | N | LYS | A | 64 | 59.244 | 13.790 | 5.761 | 1.00 | 50.45 | N |
| ATOM | 211 | CA | LYS | A | 64 | 57.864 | 13.881 | 5.295 | 1.00 | 46.99 | C |
| ATOM | 212 | C | LYS | A | 64 | 57.410 | 12.609 | 4.579 | 1.00 | 42.15 | C |
| ATOM | 213 | O | LYS | A | 64 | 57.894 | 12.284 | 3.496 | 1.00 | 41.59 | O |
| ATOM | 214 | CB | LYS | A | 64 | 57.721 | 15.079 | 4.357 | 1.00 | 47.78 | C |
| ATOM | 215 | CG | LYS | A | 64 | 56.295 | 15.412 | 3.969 | 1.00 | 52.37 | C |
| ATOM | 216 | CD | LYS | A | 64 | 56.250 | 16.756 | 3.261 | 1.00 | 56.94 | C |
| ATOM | 217 | CE | LYS | A | 64 | 54.826 | 17.248 | 3.063 | 1.00 | 60.14 | C |
| ATOM | 218 | NZ | LYS | A | 64 | 54.803 | 18.609 | 2.449 | 1.00 | 61.23 | N |
| ATOM | 219 | N | ASP | A | 65 | 56.467 | 11.899 | 5.186 | 1.00 | 35.57 | N |
| ATOM | 220 | CA | ASP | A | 65 | 55.957 | 10.665 | 4.607 | 1.00 | 32.12 | C |
| ATOM | 221 | C | ASP | A | 65 | 54.539 | 10.804 | 4.077 | 1.00 | 28.91 | C |
| ATOM | 222 | O | ASP | A | 65 | 53.832 | 11.756 | 4.392 | 1.00 | 29.02 | O |
| ATOM | 223 | CB | ASP | A | 65 | 56.006 | 9.542 | 5.644 | 1.00 | 30.47 | C |
| ATOM | 224 | CG | ASP | A | 65 | 57.413 | 9.270 | 6.138 | 1.00 | 31.78 | C |
| ATOM | 225 | OD1 | ASP | A | 65 | 58.355 | 9.364 | 5.321 | 1.00 | 31.86 | O |
| ATOM | 226 | OD2 | ASP | A | 65 | 57.581 | 8.954 | 7.333 | 1.00 | 33.41 | O |
| ATOM | 227 | N | THR | A | 66 | 54.129 | 9.842 | 3.262 | 1.00 | 28.97 | N |
| ATOM | 228 | CA | THR | A | 66 | 52.790 | 9.848 | 2.704 | 1.00 | 24.78 | C |
| ATOM | 229 | C | THR | A | 66 | 52.159 | 8.477 | 2.893 | 1.00 | 22.94 | C |
| ATOM | 230 | O | THR | A | 66 | 52.757 | 7.460 | 2.541 | 1.00 | 19.72 | O |
| ATOM | 231 | CB | THR | A | 66 | 52.821 | 10.207 | 1.214 | 1.00 | 26.41 | C |
| ATOM | 232 | OG1 | THR | A | 66 | 53.378 | 11.519 | 1.061 | 1.00 | 34.86 | O |
| ATOM | 233 | CG2 | THR | A | 66 | 51.424 | 10.202 | 0.641 | 1.00 | 28.88 | C |
| ATOM | 234 | N | PHE | A | 67 | 50.953 | 8.463 | 3.458 | 1.00 | 21.35 | N |
| ATOM | 235 | CA | PHE | A | 67 | 50.221 | 7.229 | 3.717 | 1.00 | 17.00 | C |
| ATOM | 236 | C | PHE | A | 67 | 48.950 | 7.142 | 2.876 | 1.00 | 19.27 | C |
| ATOM | 237 | O | PHE | A | 67 | 48.125 | 8.064 | 2.853 | 1.00 | 17.76 | O |
| ATOM | 238 | CB | PHE | A | 67 | 49.872 | 7.135 | 5.209 | 1.00 | 17.11 | C |
| ATOM | 239 | CG | PHE | A | 67 | 51.067 | 6.901 | 6.098 | 1.00 | 18.86 | C |
| ATOM | 240 | CD1 | PHE | A | 67 | 51.450 | 5.605 | 6.446 | 1.00 | 15.08 | C |
| ATOM | 241 | CD2 | PHE | A | 67 | 51.831 | 7.973 | 6.560 | 1.00 | 18.93 | C |
| ATOM | 242 | CE1 | PHE | A | 67 | 52.578 | 5.383 | 7.238 | 1.00 | 16.49 | C |
| ATOM | 243 | CE2 | PHE | A | 67 | 52.961 | 7.762 | 7.351 | 1.00 | 18.93 | C |
| ATOM | 244 | CZ | PHE | A | 67 | 53.335 | 6.463 | 7.690 | 1.00 | 18.60 | C |
| ATOM | 245 | N | ARG | A | 68 | 48.790 | 6.022 | 2.185 | 1.00 | 14.45 | N |
| ATOM | 246 | CA | ARG | A | 68 | 47.617 | 5.821 | 1.347 | 1.00 | 17.78 | C |
| ATOM | 247 | C | ARG | A | 68 | 46.440 | 5.301 | 2.167 | 1.00 | 17.62 | C |
| ATOM | 248 | O | ARG | A | 68 | 46.594 | 4.385 | 2.975 | 1.00 | 19.72 | O |
| ATOM | 249 | CB | ARG | A | 68 | 47.953 | 4.832 | 0.221 | 1.00 | 17.37 | C |
| ATOM | 250 | CG | ARG | A | 68 | 46.759 | 4.286 | −0.565 | 1.00 | 17.48 | C |
| ATOM | 251 | CD | ARG | A | 68 | 46.002 | 5.395 | −1.292 | 1.00 | 17.19 | C |
| ATOM | 252 | NE | ARG | A | 68 | 46.848 | 6.100 | −2.247 | 1.00 | 20.90 | N |
| ATOM | 253 | CZ | ARG | A | 68 | 47.107 | 5.679 | −3.482 | 1.00 | 24.34 | C |
| ATOM | 254 | NH1 | ARG | A | 68 | 46.580 | 4.545 | −3.933 | 1.00 | 23.76 | N |
| ATOM | 255 | NH2 | ARG | A | 68 | 47.898 | 6.396 | −4.270 | 1.00 | 22.07 | N |
| ATOM | 256 | N | VAL | A | 69 | 45.271 | 5.908 | 1.985 | 1.00 | 17.74 | N |
| ATOM | 257 | CA | VAL | A | 69 | 44.066 | 5.443 | 2.667 | 1.00 | 15.54 | C |
| ATOM | 258 | C | VAL | A | 69 | 42.949 | 5.385 | 1.640 | 1.00 | 16.48 | C |
| ATOM | 259 | O | VAL | A | 69 | 42.917 | 6.179 | 0.691 | 1.00 | 17.13 | O |
| ATOM | 260 | CB | VAL | A | 69 | 43.608 | 6.368 | 3.846 | 1.00 | 16.02 | C |
| ATOM | 261 | CG1 | VAL | A | 69 | 44.617 | 6.325 | 4.976 | 1.00 | 12.33 | C |
| ATOM | 262 | CG2 | VAL | A | 69 | 43.394 | 7.797 | 3.357 | 1.00 | 13.62 | C |
| ATOM | 263 | N | TYR | A | 70 | 42.055 | 4.420 | 1.807 | 1.00 | 14.29 | N |
| ATOM | 264 | CA | TYR | A | 70 | 40.904 | 4.274 | 0.926 | 1.00 | 16.48 | C |
| ATOM | 265 | C | TYR | A | 70 | 39.740 | 4.505 | 1.864 | 1.00 | 18.13 | C |
| ATOM | 266 | O | TYR | A | 70 | 39.704 | 3.939 | 2.957 | 1.00 | 17.21 | O |
| ATOM | 267 | CB | TYR | A | 70 | 40.870 | 2.873 | 0.316 | 1.00 | 17.29 | C |
| ATOM | 268 | CG | TYR | A | 70 | 42.055 | 2.615 | −0.591 | 1.00 | 16.17 | C |
| ATOM | 269 | CD1 | TYR | A | 70 | 42.054 | 3.048 | −1.919 | 1.00 | 17.10 | C |
| ATOM | 270 | CD2 | TYR | A | 70 | 43.195 | 1.972 | −0.109 | 1.00 | 18.55 | C |
| ATOM | 271 | CE1 | TYR | A | 70 | 43.167 | 2.845 | −2.748 | 1.00 | 16.75 | C |
| ATOM | 272 | CE2 | TYR | A | 70 | 44.313 | 1.761 | −0.923 | 1.00 | 17.03 | C |
| ATOM | 273 | CZ | TYR | A | 70 | 44.292 | 2.200 | −2.239 | 1.00 | 19.08 | C |
| ATOM | 274 | OH | TYR | A | 70 | 45.400 | 1.997 | −3.032 | 1.00 | 19.77 | O |
| ATOM | 275 | N | LYS | A | 71 | 38.785 | 5.329 | 1.446 | 1.00 | 16.21 | N |
| ATOM | 276 | CA | LYS | A | 71 | 37.681 | 5.670 | 2.327 | 1.00 | 19.31 | C |
| ATOM | 277 | C | LYS | A | 71 | 36.279 | 5.477 | 1.779 | 1.00 | 18.27 | C |
| ATOM | 278 | O | LYS | A | 71 | 36.076 | 5.352 | 0.577 | 1.00 | 16.60 | O |
| ATOM | 279 | CB | LYS | A | 71 | 37.862 | 7.121 | 2.783 | 1.00 | 21.06 | C |
| ATOM | 280 | CG | LYS | A | 71 | 39.269 | 7.399 | 3.306 | 1.00 | 25.14 | C |
| ATOM | 281 | CD | LYS | A | 71 | 39.452 | 8.839 | 3.741 | 1.00 | 27.58 | C |
| ATOM | 282 | CE | LYS | A | 71 | 39.397 | 9.781 | 2.563 | 1.00 | 30.28 | C |
| ATOM | 283 | NZ | LYS | A | 71 | 39.426 | 11.205 | 3.020 | 1.00 | 33.24 | N |
| ATOM | 284 | N | SER | A | 72 | 35.310 | 5.454 | 2.689 | 1.00 | 19.07 | N |
| ATOM | 285 | CA | SER | A | 72 | 33.917 | 5.296 | 2.310 | 1.00 | 20.59 | C |
| ATOM | 286 | C | SER | A | 72 | 32.984 | 5.733 | 3.437 | 1.00 | 22.87 | C |
| ATOM | 287 | O | SER | A | 72 | 33.335 | 5.638 | 4.617 | 1.00 | 18.52 | O |
| ATOM | 288 | CB | SER | A | 72 | 33.640 | 3.837 | 1.945 | 1.00 | 22.15 | C |
| ATOM | 289 | OG | SER | A | 72 | 32.335 | 3.697 | 1.429 | 1.00 | 31.23 | O |
| ATOM | 290 | N | GLY | A | 73 | 31.805 | 6.224 | 3.059 | 1.00 | 23.66 | N |
| ATOM | 291 | CA | GLY | A | 73 | 30.815 | 6.654 | 4.033 | 1.00 | 21.53 | C |
| ATOM | 292 | C | GLY | A | 73 | 30.935 | 8.093 | 4.501 | 1.00 | 23.18 | C |
| ATOM | 293 | O | GLY | A | 73 | 31.956 | 8.747 | 4.294 | 1.00 | 21.87 | O |
| ATOM | 294 | N | SER | A | 74 | 29.880 | 8.590 | 5.142 | 1.00 | 22.60 | N |
| ATOM | 295 | CA | SER | A | 74 | 29.887 | 9.956 | 5.651 | 1.00 | 23.44 | C |
| ATOM | 296 | C | SER | A | 74 | 29.449 | 10.044 | 7.108 | 1.00 | 23.88 | C |
| ATOM | 297 | O | SER | A | 74 | 29.808 | 10.993 | 7.809 | 1.00 | 23.86 | O |
| ATOM | 298 | CB | SER | A | 74 | 28.987 | 10.845 | 4.789 | 1.00 | 26.12 | C |
| ATOM | 299 | OG | SER | A | 74 | 27.674 | 10.316 | 4.704 | 1.00 | 28.07 | O |
| ATOM | 300 | N | GLU | A | 75 | 28.688 | 9.056 | 7.571 | 1.00 | 21.45 | N |
| ATOM | 301 | CA | GLU | A | 75 | 28.205 | 9.073 | 8.947 | 1.00 | 23.87 | C |
| ATOM | 302 | C | GLU | A | 75 | 28.517 | 7.813 | 9.737 | 1.00 | 21.78 | C |
| ATOM | 303 | O | GLU | A | 75 | 28.844 | 6.766 | 9.171 | 1.00 | 20.86 | O |
| ATOM | 304 | CB | GLU | A | 75 | 26.694 | 9.307 | 8.964 | 1.00 | 24.77 | C |
| ATOM | 305 | CG | GLU | A | 75 | 26.276 | 10.568 | 8.244 | 1.00 | 39.31 | C |
| ATOM | 306 | CD | GLU | A | 75 | 24.810 | 10.886 | 8.438 | 1.00 | 43.49 | C |
| ATOM | 307 | OE1 | GLU | A | 75 | 24.436 | 11.315 | 9.552 | 1.00 | 46.86 | O |
| ATOM | 308 | OE2 | GLU | A | 75 | 24.035 | 10.697 | 7.476 | 1.00 | 46.55 | O |
| ATOM | 309 | N | GLY | A | 76 | 28.391 | 7.919 | 11.054 | 1.00 | 18.65 | N |
| ATOM | 310 | CA | GLY | A | 76 | 28.666 | 6.783 | 11.906 | 1.00 | 15.76 | C |
| ATOM | 311 | C | GLY | A | 76 | 30.137 | 6.689 | 12.254 | 1.00 | 16.03 | C |
| ATOM | 312 | O | GLY | A | 76 | 30.952 | 7.493 | 11.795 | 1.00 | 16.95 | O |
| ATOM | 313 | N | PRO | A | 77 | 30.515 | 5.695 | 13.061 | 1.00 | 16.80 | N |
| ATOM | 314 | CA | PRO | A | 77 | 31.911 | 5.524 | 13.463 | 1.00 | 16.71 | C |
| ATOM | 315 | C | PRO | A | 77 | 32.829 | 5.175 | 12.311 | 1.00 | 12.58 | C |
| ATOM | 316 | O | PRO | A | 77 | 32.430 | 4.520 | 11.354 | 1.00 | 17.71 | O |
| ATOM | 317 | CB | PRO | A | 77 | 31.837 | 4.390 | 14.479 | 1.00 | 13.95 | C |
| ATOM | 318 | CG | PRO | A | 77 | 30.742 | 3.543 | 13.921 | 1.00 | 19.44 | C |
| ATOM | 319 | CD | PRO | A | 77 | 29.689 | 4.572 | 13.540 | 1.00 | 16.68 | C |
| ATOM | 320 | N | VAL | A | 78 | 34.065 | 5.632 | 12.417 | 1.00 | 12.58 | N |
| ATOM | 321 | CA | VAL | A | 78 | 35.067 | 5.320 | 11.418 | 1.00 | 12.80 | C |
| ATOM | 322 | C | VAL | A | 78 | 35.712 | 4.009 | 11.852 | 1.00 | 14.86 | C |
| ATOM | 323 | O | VAL | A | 78 | 36.186 | 3.877 | 12.988 | 1.00 | 13.63 | O |
| ATOM | 324 | CB | VAL | A | 78 | 36.154 | 6.419 | 11.348 | 1.00 | 14.09 | C |
| ATOM | 325 | CG1 | VAL | A | 78 | 37.443 | 5.857 | 10.740 | 1.00 | 18.51 | C |
| ATOM | 326 | CG2 | VAL | A | 78 | 35.649 | 7.586 | 10.503 | 1.00 | 13.93 | C |
| ATOM | 327 | N | LEU | A | 79 | 35.688 | 3.032 | 10.961 | 1.00 | 12.76 | N |
| ATOM | 328 | CA | LEU | A | 79 | 36.321 | 1.744 | 11.216 | 1.00 | 12.01 | C |
| ATOM | 329 | C | LEU | A | 79 | 37.701 | 1.872 | 10.561 | 1.00 | 12.22 | C |
| ATOM | 330 | O | LEU | A | 79 | 37.808 | 1.903 | 9.341 | 1.00 | 14.90 | O |
| ATOM | 331 | CB | LEU | A | 79 | 35.498 | 0.623 | 10.561 | 1.00 | 13.99 | C |
| ATOM | 332 | CG | LEU | A | 79 | 34.277 | 0.076 | 11.320 | 1.00 | 22.29 | C |
| ATOM | 333 | CD1 | LEU | A | 79 | 33.531 | 1.167 | 12.052 | 1.00 | 24.24 | C |
| ATOM | 334 | CD2 | LEU | A | 79 | 33.376 | −0.669 | 10.341 | 1.00 | 17.32 | C |
| ATOM | 335 | N | LEU | A | 80 | 38.743 | 2.001 | 11.380 | 1.00 | 12.50 | N |
| ATOM | 336 | CA | LEU | A | 80 | 40.110 | 2.153 | 10.892 | 1.00 | 13.66 | C |
| ATOM | 337 | C | LEU | A | 80 | 40.693 | 0.748 | 10.750 | 1.00 | 13.79 | C |
| ATOM | 338 | O | LEU | A | 80 | 41.026 | 0.094 | 11.741 | 1.00 | 13.47 | O |
| ATOM | 339 | CB | LEU | A | 80 | 40.914 | 3.003 | 11.886 | 1.00 | 11.31 | C |
| ATOM | 340 | CG | LEU | A | 80 | 42.369 | 3.357 | 11.569 | 1.00 | 13.76 | C |
| ATOM | 341 | CD1 | LEU | A | 80 | 42.493 | 4.004 | 10.192 | 1.00 | 10.56 | C |
| ATOM | 342 | CD2 | LEU | A | 80 | 42.885 | 4.300 | 12.658 | 1.00 | 15.29 | C |
| ATOM | 343 | N | LEU | A | 81 | 40.812 | 0.302 | 9.504 | 1.00 | 13.43 | N |
| ATOM | 344 | CA | LEU | A | 81 | 41.262 | −1.057 | 9.194 | 1.00 | 16.16 | C |
| ATOM | 345 | C | LEU | A | 81 | 42.747 | −1.202 | 8.905 | 1.00 | 14.83 | C |
| ATOM | 346 | O | LEU | A | 81 | 43.298 | −0.519 | 8.033 | 1.00 | 13.15 | O |
| ATOM | 347 | CB | LEU | A | 81 | 40.452 | −1.586 | 8.006 | 1.00 | 12.00 | C |
| ATOM | 348 | CG | LEU | A | 81 | 38.968 | −1.193 | 8.008 | 1.00 | 14.67 | C |
| ATOM | 349 | CD1 | LEU | A | 81 | 38.291 | −1.712 | 6.744 | 1.00 | 13.16 | C |
| ATOM | 350 | CD2 | LEU | A | 81 | 38.276 | −1.753 | 9.256 | 1.00 | 12.99 | C |
| ATOM | 351 | N | LEU | A | 82 | 43.378 | −2.126 | 9.626 | 1.00 | 13.59 | N |
| ATOM | 352 | CA | LEU | A | 82 | 44.807 | −2.383 | 9.494 | 1.00 | 13.68 | C |
| ATOM | 353 | C | LEU | A | 82 | 45.050 | −3.814 | 9.000 | 1.00 | 14.41 | C |
| ATOM | 354 | O | LEU | A | 82 | 44.738 | −4.782 | 9.690 | 1.00 | 13.60 | O |
| ATOM | 355 | CB | LEU | A | 82 | 45.493 | −2.169 | 10.847 | 1.00 | 11.55 | C |
| ATOM | 356 | CG | LEU | A | 82 | 45.242 | −0.827 | 11.556 | 1.00 | 12.51 | C |
| ATOM | 357 | CD1 | LEU | A | 82 | 46.134 | −0.745 | 12.799 | 1.00 | 10.88 | C |
| ATOM | 358 | CD2 | LEU | A | 82 | 45.563 | 0.346 | 10.608 | 1.00 | 12.49 | C |
| ATOM | 359 | N | HIS | A | 83 | 45.612 | −3.917 | 7.803 | 1.00 | 11.46 | N |
| ATOM | 360 | CA | HIS | A | 83 | 45.900 | −5.184 | 7.141 | 1.00 | 12.10 | C |
| ATOM | 361 | C | HIS | A | 83 | 47.076 | −5.951 | 7.753 | 1.00 | 12.73 | C |
| ATOM | 362 | O | HIS | A | 83 | 47.810 | −5.434 | 8.606 | 1.00 | 10.15 | O |
| ATOM | 363 | CB | HIS | A | 83 | 46.200 | −4.910 | 5.667 | 1.00 | 12.39 | C |
| ATOM | 364 | CG | HIS | A | 83 | 47.421 | −4.068 | 5.458 | 1.00 | 15.10 | C |
| ATOM | 365 | ND1 | HIS | A | 83 | 48.697 | −4.558 | 5.632 | 1.00 | 15.34 | N |
| ATOM | 366 | CD2 | HIS | A | 83 | 47.559 | −2.752 | 5.172 | 1.00 | 13.07 | C |
| ATOM | 367 | CE1 | HIS | A | 83 | 49.569 | −3.578 | 5.468 | 1.00 | 12.74 | C |
| ATOM | 368 | NE2 | HIS | A | 83 | 48.904 | −2.472 | 5.190 | 1.00 | 15.28 | N |
| ATOM | 369 | N | GLY | A | 84 | 47.254 | −7.182 | 7.281 | 1.00 | 15.18 | N |
| ATOM | 370 | CA | GLY | A | 84 | 48.325 | −8.026 | 7.765 | 1.00 | 14.68 | C |
| ATOM | 371 | C | GLY | A | 84 | 49.607 | −7.875 | 6.970 | 1.00 | 15.78 | C |
| ATOM | 372 | O | GLY | A | 84 | 49.658 | −7.159 | 5.964 | 1.00 | 15.42 | O |
| ATOM | 373 | N | GLY | A | 85 | 50.644 | −8.572 | 7.416 | 1.00 | 15.22 | N |
| ATOM | 374 | CA | GLY | A | 85 | 51.932 | −8.487 | 6.750 | 1.00 | 15.84 | C |
| ATOM | 375 | C | GLY | A | 85 | 51.889 | −8.855 | 5.285 | 1.00 | 16.48 | C |
| ATOM | 376 | O | GLY | A | 85 | 51.273 | −9.851 | 4.906 | 1.00 | 16.92 | O |
| ATOM | 377 | N | GLY | A | 86 | 52.533 | −8.038 | 4.460 | 1.00 | 15.35 | N |
| ATOM | 378 | CA | GLY | A | 86 | 52.582 | −8.309 | 3.034 | 1.00 | 17.54 | C |
| ATOM | 379 | C | GLY | A | 86 | 51.307 | −7.998 | 2.277 | 1.00 | 20.45 | C |
| ATOM | 380 | O | GLY | A | 86 | 51.256 | −8.164 | 1.058 | 1.00 | 21.59 | O |
| ATOM | 381 | N | HIS | A | 87 | 50.271 | −7.559 | 2.986 | 1.00 | 14.88 | N |
| ATOM | 382 | CA | HIS | A | 87 | 49.010 | −7.226 | 2.337 | 1.00 | 14.07 | C |
| ATOM | 383 | C | HIS | A | 87 | 48.889 | −5.715 | 2.167 | 1.00 | 16.32 | C |
| ATOM | 384 | O | HIS | A | 87 | 49.898 | −5.012 | 2.158 | 1.00 | 15.25 | O |
| ATOM | 385 | CB | HIS | A | 87 | 47.843 | −7.784 | 3.151 | 1.00 | 18.94 | C |
| ATOM | 386 | CG | HIS | A | 87 | 47.765 | −9.279 | 3.139 | 1.00 | 27.39 | C |
| ATOM | 387 | ND1 | HIS | A | 87 | 46.745 | −9.979 | 3.748 | 1.00 | 32.21 | N |
| ATOM | 388 | CD2 | HIS | A | 87 | 48.579 | −10.210 | 2.585 | 1.00 | 31.04 | C |
| ATOM | 389 | CE1 | HIS | A | 87 | 46.932 | −11.275 | 3.567 | 1.00 | 30.28 | C |
| ATOM | 390 | NE2 | HIS | A | 87 | 48.038 | −11.442 | 2.865 | 1.00 | 31.40 | N |
| ATOM | 391 | N | SER | A | 88 | 47.663 | −5.216 | 2.028 | 1.00 | 16.80 | N |
| ATOM | 392 | CA | SER | A | 88 | 47.436 | −3.782 | 1.852 | 1.00 | 15.90 | C |
| ATOM | 393 | C | SER | A | 88 | 46.011 | −3.456 | 2.271 | 1.00 | 14.50 | C |
| ATOM | 394 | O | SER | A | 88 | 45.242 | −4.352 | 2.607 | 1.00 | 16.33 | O |
| ATOM | 395 | CB | SER | A | 88 | 47.612 | −3.383 | 0.387 | 1.00 | 16.04 | C |
| ATOM | 396 | OG | SER | A | 88 | 46.449 | −3.724 | −0.358 | 1.00 | 19.36 | O |
| ATOM | 397 | N | ALA | A | 89 | 45.658 | −2.173 | 2.230 | 1.00 | 14.46 | N |
| ATOM | 398 | CA | ALA | A | 89 | 44.315 | −1.741 | 2.602 | 1.00 | 15.56 | C |
| ATOM | 399 | C | ALA | A | 89 | 43.280 | −2.325 | 1.651 | 1.00 | 12.96 | C |
| ATOM | 400 | O | ALA | A | 89 | 42.108 | −2.465 | 2.005 | 1.00 | 15.37 | O |
| ATOM | 401 | CB | ALA | A | 89 | 44.229 | −0.210 | 2.591 | 1.00 | 13.65 | C |
| ATOM | 402 | N | LEU | A | 90 | 43.706 | −2.664 | 0.440 | 1.00 | 13.51 | N |
| ATOM | 403 | CA | LEU | A | 90 | 42.781 | −3.228 | −0.541 | 1.00 | 14.11 | C |
| ATOM | 404 | C | LEU | A | 90 | 42.256 | −4.607 | −0.130 | 1.00 | 17.34 | C |
| ATOM | 405 | O | LEU | A | 90 | 41.275 | −5.097 | −0.688 | 1.00 | 13.98 | O |
| ATOM | 406 | CB | LEU | A | 90 | 43.455 | −3.286 | −1.916 | 1.00 | 16.93 | C |
| ATOM | 407 | CG | LEU | A | 90 | 43.812 | −1.884 | −2.427 | 1.00 | 14.59 | C |
| ATOM | 408 | CD1 | LEU | A | 90 | 44.569 | −1.974 | −3.747 | 1.00 | 18.15 | C |
| ATOM | 409 | CD2 | LEU | A | 90 | 42.533 | −1.078 | −2.596 | 1.00 | 18.35 | C |
| ATOM | 410 | N | SER | A | 91 | 42.911 | −5.230 | 0.848 | 1.00 | 16.73 | N |
| ATOM | 411 | CA | SER | A | 91 | 42.470 | −6.532 | 1.352 | 1.00 | 17.00 | C |
| ATOM | 412 | C | SER | A | 91 | 41.121 | −6.372 | 2.056 | 1.00 | 14.98 | C |
| ATOM | 413 | O | SER | A | 91 | 40.409 | −7.350 | 2.275 | 1.00 | 15.03 | O |
| ATOM | 414 | CB | SER | A | 91 | 43.492 | −7.101 | 2.349 | 1.00 | 16.36 | C |
| ATOM | 415 | OG | SER | A | 91 | 44.693 | −7.464 | 1.684 | 1.00 | 25.32 | O |
| ATOM | 416 | N | TRP | A | 92 | 40.780 | −5.134 | 2.405 | 1.00 | 12.45 | N |
| ATOM | 417 | CA | TRP | A | 92 | 39.513 | −4.837 | 3.084 | 1.00 | 14.71 | C |
| ATOM | 418 | C | TRP | A | 92 | 38.396 | −4.377 | 2.133 | 1.00 | 15.24 | C |
| ATOM | 419 | O | TRP | A | 92 | 37.303 | −4.009 | 2.577 | 1.00 | 15.17 | O |
| ATOM | 420 | CB | TRP | A | 92 | 39.725 | −3.733 | 4.130 | 1.00 | 13.38 | C |
| ATOM | 421 | CG | TRP | A | 92 | 40.726 | −4.073 | 5.187 | 1.00 | 11.92 | C |
| ATOM | 422 | CD1 | TRP | A | 92 | 42.028 | −3.671 | 5.246 | 1.00 | 13.66 | C |
| ATOM | 423 | CD2 | TRP | A | 92 | 40.487 | −4.840 | 6.369 | 1.00 | 11.51 | C |
| ATOM | 424 | NE1 | TRP | A | 92 | 42.614 | −4.133 | 6.401 | 1.00 | 12.06 | N |
| ATOM | 425 | CE2 | TRP | A | 92 | 41.690 | −4.855 | 7.108 | 1.00 | 14.44 | C |
| ATOM | 426 | CE3 | TRP | A | 92 | 39.369 | −5.515 | 6.878 | 1.00 | 13.39 | C |
| ATOM | 427 | CZ2 | TRP | A | 92 | 41.809 | −5.521 | 8.337 | 1.00 | 13.95 | C |
| ATOM | 428 | CZ3 | TRP | A | 92 | 39.486 | −6.179 | 8.107 | 1.00 | 14.39 | C |
| ATOM | 429 | CH2 | TRP | A | 92 | 40.699 | −6.173 | 8.817 | 1.00 | 13.93 | C |
| ATOM | 430 | N | ALA | A | 93 | 38.662 | −4.390 | 0.834 | 1.00 | 15.61 | N |
| ATOM | 431 | CA | ALA | A | 93 | 37.668 | −3.925 | −0.135 | 1.00 | 17.49 | C |
| ATOM | 432 | C | ALA | A | 93 | 36.304 | −4.614 | −0.053 | 1.00 | 19.18 | C |
| ATOM | 433 | O | ALA | A | 93 | 35.279 | −3.950 | 0.128 | 1.00 | 16.64 | O |
| ATOM | 434 | CB | ALA | A | 93 | 38.227 | −4.040 | −1.550 | 1.00 | 16.12 | C |
| ATOM | 435 | N | VAL | A | 94 | 36.277 | −5.937 | −0.178 | 1.00 | 17.88 | N |
| ATOM | 436 | CA | VAL | A | 94 | 35.002 | −6.647 | −0.133 | 1.00 | 18.64 | C |
| ATOM | 437 | C | VAL | A | 94 | 34.334 | −6.537 | 1.238 | 1.00 | 16.99 | C |
| ATOM | 438 | O | VAL | A | 94 | 33.109 | −6.462 | 1.338 | 1.00 | 16.70 | O |
| ATOM | 439 | CB | VAL | A | 94 | 35.170 | −8.132 | −0.526 | 1.00 | 17.56 | C |
| ATOM | 440 | CG1 | VAL | A | 94 | 33.809 | −8.811 | −0.591 | 1.00 | 21.76 | C |
| ATOM | 441 | CG2 | VAL | A | 94 | 35.847 | −8.225 | −1.884 | 1.00 | 21.82 | C |
| ATOM | 442 | N | PHE | A | 95 | 35.142 | −6.509 | 2.290 | 1.00 | 17.05 | N |
| ATOM | 443 | CA | PHE | A | 95 | 34.616 | −6.382 | 3.642 | 1.00 | 14.67 | C |
| ATOM | 444 | C | PHE | A | 95 | 33.895 | −5.047 | 3.786 | 1.00 | 17.00 | C |
| ATOM | 445 | O | PHE | A | 95 | 32.838 | −4.960 | 4.417 | 1.00 | 15.98 | O |
| ATOM | 446 | CB | PHE | A | 95 | 35.753 | −6.431 | 4.665 | 1.00 | 13.58 | C |
| ATOM | 447 | CG | PHE | A | 95 | 35.363 | −5.918 | 6.027 | 1.00 | 12.98 | C |
| ATOM | 448 | CD1 | PHE | A | 95 | 34.644 | −6.720 | 6.910 | 1.00 | 12.18 | C |
| ATOM | 449 | CD2 | PHE | A | 95 | 35.686 | −4.613 | 6.412 | 1.00 | 14.18 | C |
| ATOM | 450 | CE1 | PHE | A | 95 | 34.248 | −6.229 | 8.162 | 1.00 | 16.35 | C |
| ATOM | 451 | CE2 | PHE | A | 95 | 35.296 | −4.112 | 7.656 | 1.00 | 15.12 | C |
| ATOM | 452 | CZ | PHE | A | 95 | 34.575 | −4.923 | 8.534 | 1.00 | 17.40 | C |
| ATOM | 453 | N | THR | A | 96 | 34.495 | −4.013 | 3.205 | 1.00 | 17.47 | N |
| ATOM | 454 | CA | THR | A | 96 | 33.961 | −2.657 | 3.265 | 1.00 | 17.86 | C |
| ATOM | 455 | C | THR | A | 96 | 32.549 | −2.576 | 2.702 | 1.00 | 17.34 | C |
| ATOM | 456 | O | THR | A | 96 | 31.667 | −1.980 | 3.319 | 1.00 | 17.25 | O |
| ATOM | 457 | CB | THR | A | 96 | 34.891 | −1.667 | 2.512 | 1.00 | 17.90 | C |
| ATOM | 458 | OG1 | THR | A | 96 | 36.159 | −1.590 | 3.187 | 1.00 | 17.94 | O |
| ATOM | 459 | CG2 | THR | A | 96 | 34.280 | −0.274 | 2.468 | 1.00 | 16.30 | C |
| ATOM | 460 | N | ALA | A | 97 | 32.335 | −3.178 | 1.536 | 1.00 | 19.75 | N |
| ATOM | 461 | CA | ALA | A | 97 | 31.019 | −3.167 | 0.905 | 1.00 | 21.84 | C |
| ATOM | 462 | C | ALA | A | 97 | 30.024 | −3.923 | 1.776 | 1.00 | 21.63 | C |
| ATOM | 463 | O | ALA | A | 97 | 28.876 | −3.505 | 1.934 | 1.00 | 21.71 | O |
| ATOM | 464 | CB | ALA | A | 97 | 31.094 | −3.805 | −0.485 | 1.00 | 21.02 | C |
| ATOM | 465 | N | ALA | A | 98 | 30.474 | −5.034 | 2.344 | 1.00 | 19.20 | N |
| ATOM | 466 | CA | ALA | A | 98 | 29.627 | −5.848 | 3.200 | 1.00 | 19.89 | C |
| ATOM | 467 | C | ALA | A | 98 | 29.221 | −5.132 | 4.491 | 1.00 | 18.39 | C |
| ATOM | 468 | O | ALA | A | 98 | 28.042 | −5.126 | 4.845 | 1.00 | 23.28 | O |
| ATOM | 469 | CB | ALA | A | 98 | 30.330 | −7.166 | 3.533 | 1.00 | 16.92 | C |
| ATOM | 470 | N | ILE | A | 99 | 30.174 | −4.514 | 5.188 | 1.00 | 17.25 | N |
| ATOM | 471 | CA | ILE | A | 99 | 29.843 | −3.840 | 6.447 | 1.00 | 16.23 | C |
| ATOM | 472 | C | ILE | A | 99 | 28.992 | −2.581 | 6.232 | 1.00 | 18.92 | C |
| ATOM | 473 | O | ILE | A | 99 | 28.064 | −2.305 | 7.003 | 1.00 | 20.37 | O |
| ATOM | 474 | CB | ILE | A | 99 | 31.131 | −3.500 | 7.273 | 1.00 | 16.19 | C |
| ATOM | 475 | CG1 | ILE | A | 99 | 30.744 | −3.013 | 8.673 | 1.00 | 20.91 | C |
| ATOM | 476 | CG2 | ILE | A | 99 | 31.958 | −2.424 | 6.578 | 1.00 | 13.46 | C |
| ATOM | 477 | CD1 | ILE | A | 99 | 30.109 | −4.075 | 9.546 | 1.00 | 22.10 | C |
| ATOM | 478 | N | ILE | A | 100 | 29.294 | −1.830 | 5.177 | 1.00 | 19.39 | N |
| ATOM | 479 | CA | ILE | A | 100 | 28.540 | −0.622 | 4.854 | 1.00 | 22.82 | C |
| ATOM | 480 | C | ILE | A | 100 | 27.059 | −0.940 | 4.616 | 1.00 | 23.64 | C |
| ATOM | 481 | O | ILE | A | 100 | 26.186 | −0.112 | 4.887 | 1.00 | 22.19 | O |
| ATOM | 482 | CB | ILE | A | 100 | 29.101 | 0.069 | 3.579 | 1.00 | 26.63 | C |
| ATOM | 483 | CG1 | ILE | A | 100 | 30.468 | 0.691 | 3.873 | 1.00 | 25.14 | C |
| ATOM | 484 | CG2 | ILE | A | 100 | 28.116 | 1.119 | 3.073 | 1.00 | 26.45 | C |
| ATOM | 485 | CD1 | ILE | A | 100 | 30.439 | 1.807 | 4.875 | 1.00 | 33.00 | C |
| ATOM | 486 | N | SER | A | 101 | 26.777 | −2.138 | 4.116 | 1.00 | 20.74 | N |
| ATOM | 487 | CA | SER | A | 101 | 25.402 | −2.528 | 3.842 | 1.00 | 23.15 | C |
| ATOM | 488 | C | SER | A | 101 | 24.666 | −3.015 | 5.086 | 1.00 | 23.36 | C |
| ATOM | 489 | O | SER | A | 101 | 23.459 | −3.244 | 5.042 | 1.00 | 23.81 | O |
| ATOM | 490 | CB | SER | A | 101 | 25.362 | −3.623 | 2.772 | 1.00 | 27.39 | C |
| ATOM | 491 | OG | SER | A | 101 | 25.783 | −4.873 | 3.294 | 1.00 | 28.19 | O |
| ATOM | 492 | N | ARG | A | 102 | 25.386 | −3.159 | 6.196 | 1.00 | 23.24 | N |
| ATOM | 493 | CA | ARG | A | 102 | 24.782 | −3.646 | 7.435 | 1.00 | 22.62 | C |
| ATOM | 494 | C | ARG | A | 102 | 24.609 | −2.578 | 8.509 | 1.00 | 22.41 | C |
| ATOM | 495 | O | ARG | A | 102 | 23.633 | −2.593 | 9.249 | 1.00 | 24.35 | O |
| ATOM | 496 | CB | ARG | A | 102 | 25.621 | −4.788 | 8.005 | 1.00 | 23.58 | C |
| ATOM | 497 | CG | ARG | A | 102 | 25.933 | −5.877 | 7.007 | 1.00 | 27.42 | C |
| ATOM | 498 | CD | ARG | A | 102 | 26.825 | −6.934 | 7.627 | 1.00 | 31.55 | C |
| ATOM | 499 | NE | ARG | A | 102 | 26.038 | −7.985 | 8.251 | 1.00 | 41.62 | N |
| ATOM | 500 | CZ | ARG | A | 102 | 25.563 | −9.037 | 7.600 | 1.00 | 40.96 | C |
| ATOM | 501 | NH1 | ARG | A | 102 | 25.808 | −9.178 | 6.307 | 1.00 | 43.94 | N |
| ATOM | 502 | NH2 | ARG | A | 102 | 24.831 | −9.938 | 8.238 | 1.00 | 45.08 | N |
| ATOM | 503 | N | VAL | A | 103 | 25.570 | −1.666 | 8.613 | 1.00 | 21.74 | N |
| ATOM | 504 | CA | VAL | A | 103 | 25.496 | −0.606 | 9.612 | 1.00 | 19.51 | C |
| ATOM | 505 | C | VAL | A | 103 | 25.916 | 0.724 | 9.015 | 1.00 | 20.12 | C |
| ATOM | 506 | O | VAL | A | 103 | 26.676 | 0.766 | 8.045 | 1.00 | 20.27 | O |
| ATOM | 507 | CB | VAL | A | 103 | 26.418 | −0.890 | 10.827 | 1.00 | 21.02 | C |
| ATOM | 508 | CG1 | VAL | A | 103 | 25.955 | −2.138 | 11.549 | 1.00 | 19.96 | C |
| ATOM | 509 | CG2 | VAL | A | 103 | 27.868 | −1.052 | 10.362 | 1.00 | 19.70 | C |
| ATOM | 510 | N | GLN | A | 104 | 25.410 | 1.808 | 9.596 | 1.00 | 17.40 | N |
| ATOM | 511 | CA | GLN | A | 104 | 25.752 | 3.146 | 9.140 | 1.00 | 20.71 | C |
| ATOM | 512 | C | GLN | A | 104 | 27.133 | 3.429 | 9.707 | 1.00 | 17.94 | C |
| ATOM | 513 | O | GLN | A | 104 | 27.292 | 3.566 | 10.919 | 1.00 | 15.90 | O |
| ATOM | 514 | CB | GLN | A | 104 | 24.749 | 4.165 | 9.684 | 1.00 | 26.52 | C |
| ATOM | 515 | CG | GLN | A | 104 | 24.933 | 5.578 | 9.151 | 1.00 | 35.97 | C |
| ATOM | 516 | CD | GLN | A | 104 | 24.607 | 5.685 | 7.672 | 1.00 | 45.56 | C |
| ATOM | 517 | OE1 | GLN | A | 104 | 23.554 | 5.224 | 7.221 | 1.00 | 49.23 | O |
| ATOM | 518 | NE2 | GLN | A | 104 | 25.504 | 6.302 | 6.908 | 1.00 | 47.64 | N |
| ATOM | 519 | N | CYS | A | 105 | 28.134 | 3.510 | 8.837 | 1.00 | 16.49 | N |
| ATOM | 520 | CA | CYS | A | 105 | 29.492 | 3.756 | 9.299 | 1.00 | 16.92 | C |
| ATOM | 521 | C | CYS | A | 105 | 30.377 | 4.300 | 8.201 | 1.00 | 17.62 | C |
| ATOM | 522 | O | CYS | A | 105 | 29.959 | 4.418 | 7.049 | 1.00 | 18.81 | O |
| ATOM | 523 | CB | CYS | A | 105 | 30.110 | 2.459 | 9.825 | 1.00 | 18.03 | C |
| ATOM | 524 | SG | CYS | A | 105 | 30.377 | 1.190 | 8.546 | 1.00 | 21.68 | S |
| ATOM | 525 | N | ARG | A | 106 | 31.603 | 4.641 | 8.583 | 1.00 | 15.64 | N |
| ATOM | 526 | CA | ARG | A | 106 | 32.606 | 5.146 | 7.657 | 1.00 | 16.90 | C |
| ATOM | 527 | C | ARG | A | 106 | 33.762 | 4.162 | 7.703 | 1.00 | 17.48 | C |
| ATOM | 528 | O | ARG | A | 106 | 33.913 | 3.405 | 8.663 | 1.00 | 16.88 | O |
| ATOM | 529 | CB | ARG | A | 106 | 33.092 | 6.545 | 8.069 | 1.00 | 15.73 | C |
| ATOM | 530 | CG | ARG | A | 106 | 32.135 | 7.673 | 7.666 | 1.00 | 18.66 | C |
| ATOM | 531 | CD | ARG | A | 106 | 32.535 | 9.017 | 8.284 | 1.00 | 15.95 | C |
| ATOM | 532 | NE | ARG | A | 106 | 32.109 | 9.115 | 9.680 | 1.00 | 17.87 | N |
| ATOM | 533 | CZ | ARG | A | 106 | 32.316 | 10.171 | 10.461 | 1.00 | 17.57 | C |
| ATOM | 534 | NH1 | ARG | A | 106 | 32.951 | 11.240 | 9.999 | 1.00 | 15.67 | N |
| ATOM | 535 | NH2 | ARG | A | 106 | 31.879 | 10.160 | 11.712 | 1.00 | 22.56 | N |
| ATOM | 536 | N | ILE | A | 107 | 34.571 | 4.170 | 6.656 | 1.00 | 20.27 | N |
| ATOM | 537 | CA | ILE | A | 107 | 35.707 | 3.272 | 6.575 | 1.00 | 18.86 | C |
| ATOM | 538 | C | ILE | A | 107 | 36.966 | 4.029 | 6.185 | 1.00 | 16.86 | C |
| ATOM | 539 | O | ILE | A | 107 | 36.940 | 4.910 | 5.327 | 1.00 | 15.74 | O |
| ATOM | 540 | CB | ILE | A | 107 | 35.501 | 2.178 | 5.486 | 1.00 | 20.39 | C |
| ATOM | 541 | CG1 | ILE | A | 107 | 34.218 | 1.378 | 5.745 | 1.00 | 24.65 | C |
| ATOM | 542 | CG2 | ILE | A | 107 | 36.724 | 1.263 | 5.433 | 1.00 | 22.55 | C |
| ATOM | 543 | CD1 | ILE | A | 107 | 34.266 | 0.472 | 6.950 | 1.00 | 23.53 | C |
| ATOM | 544 | N | VAL | A | 108 | 38.064 | 3.699 | 6.846 | 1.00 | 14.40 | N |
| ATOM | 545 | CA | VAL | A | 108 | 39.355 | 4.264 | 6.488 | 1.00 | 13.87 | C |
| ATOM | 546 | C | VAL | A | 108 | 40.298 | 3.061 | 6.533 | 1.00 | 14.20 | C |
| ATOM | 547 | O | VAL | A | 108 | 40.654 | 2.588 | 7.609 | 1.00 | 14.76 | O |
| ATOM | 548 | CB | VAL | A | 108 | 39.830 | 5.363 | 7.462 | 1.00 | 13.67 | C |
| ATOM | 549 | CG1 | VAL | A | 108 | 41.234 | 5.834 | 7.065 | 1.00 | 13.75 | C |
| ATOM | 550 | CG2 | VAL | A | 108 | 38.873 | 6.563 | 7.414 | 1.00 | 12.02 | C |
| ATOM | 551 | N | ALA | A | 109 | 40.639 | 2.537 | 5.354 | 1.00 | 14.51 | N |
| ATOM | 552 | CA | ALA | A | 109 | 41.552 | 1.399 | 5.222 | 1.00 | 15.60 | C |
| ATOM | 553 | C | ALA | A | 109 | 42.931 | 1.975 | 4.921 | 1.00 | 14.81 | C |
| ATOM | 554 | O | ALA | A | 109 | 43.129 | 2.635 | 3.894 | 1.00 | 15.27 | O |
| ATOM | 555 | CB | ALA | A | 109 | 41.097 | 0.484 | 4.089 | 1.00 | 13.34 | C |
| ATOM | 556 | N | LEU | A | 110 | 43.878 | 1.712 | 5.813 | 1.00 | 13.25 | N |
| ATOM | 557 | CA | LEU | A | 110 | 45.239 | 2.242 | 5.712 | 1.00 | 14.57 | C |
| ATOM | 558 | C | LEU | A | 110 | 46.350 | 1.299 | 5.237 | 1.00 | 16.87 | C |
| ATOM | 559 | O | LEU | A | 110 | 46.477 | 0.177 | 5.732 | 1.00 | 14.00 | O |
| ATOM | 560 | CB | LEU | A | 110 | 45.638 | 2.802 | 7.086 | 1.00 | 14.39 | C |
| ATOM | 561 | CG | LEU | A | 110 | 47.068 | 3.283 | 7.338 | 1.00 | 16.68 | C |
| ATOM | 562 | CD1 | LEU | A | 110 | 47.361 | 4.513 | 6.493 | 1.00 | 16.15 | C |
| ATOM | 563 | CD2 | LEU | A | 110 | 47.235 | 3.620 | 8.817 | 1.00 | 16.09 | C |
| ATOM | 564 | N | ASP | A | 111 | 47.151 | 1.767 | 4.280 | 1.00 | 16.65 | N |
| ATOM | 565 | CA | ASP | A | 111 | 48.307 | 1.004 | 3.803 | 1.00 | 16.41 | C |
| ATOM | 566 | C | ASP | A | 111 | 49.380 | 1.323 | 4.830 | 1.00 | 14.94 | C |
| ATOM | 567 | O | ASP | A | 111 | 49.809 | 2.470 | 4.944 | 1.00 | 16.64 | O |
| ATOM | 568 | CB | ASP | A | 111 | 48.811 | 1.497 | 2.442 | 1.00 | 17.13 | C |
| ATOM | 569 | CG | ASP | A | 111 | 48.033 | 0.921 | 1.283 | 1.00 | 18.22 | C |
| ATOM | 570 | OD1 | ASP | A | 111 | 47.278 | −0.046 | 1.494 | 1.00 | 17.56 | O |
| ATOM | 571 | OD2 | ASP | A | 111 | 48.196 | 1.434 | 0.152 | 1.00 | 19.22 | O |
| ATOM | 572 | N | LEU | A | 112 | 49.804 | 0.326 | 5.591 | 1.00 | 14.27 | N |
| ATOM | 573 | CA | LEU | A | 112 | 50.817 | 0.563 | 6.601 | 1.00 | 15.35 | C |
| ATOM | 574 | C | LEU | A | 112 | 52.184 | 0.801 | 5.963 | 1.00 | 16.26 | C |
| ATOM | 575 | O | LEU | A | 112 | 52.370 | 0.592 | 4.766 | 1.00 | 17.33 | O |
| ATOM | 576 | CB | LEU | A | 112 | 50.867 | −0.620 | 7.571 | 1.00 | 16.09 | C |
| ATOM | 577 | CG | LEU | A | 112 | 49.587 | −0.788 | 8.395 | 1.00 | 16.55 | C |
| ATOM | 578 | CD1 | LEU | A | 112 | 49.535 | −2.174 | 9.023 | 1.00 | 13.54 | C |
| ATOM | 579 | CD2 | LEU | A | 112 | 49.531 | 0.309 | 9.461 | 1.00 | 17.88 | C |
| ATOM | 580 | N | ARG | A | 113 | 53.122 | 1.277 | 6.769 | 1.00 | 15.99 | N |
| ATOM | 581 | CA | ARG | A | 113 | 54.481 | 1.553 | 6.323 | 1.00 | 19.30 | C |
| ATOM | 582 | C | ARG | A | 113 | 55.025 | 0.389 | 5.482 | 1.00 | 20.64 | C |
| ATOM | 583 | O | ARG | A | 113 | 54.870 | −0.777 | 5.850 | 1.00 | 19.59 | O |
| ATOM | 584 | CB | ARG | A | 113 | 55.367 | 1.788 | 7.557 | 1.00 | 17.93 | C |
| ATOM | 585 | CG | ARG | A | 113 | 56.850 | 1.939 | 7.296 | 1.00 | 25.89 | C |
| ATOM | 586 | CD | ARG | A | 113 | 57.591 | 2.294 | 8.592 | 1.00 | 26.06 | C |
| ATOM | 587 | NE | ARG | A | 113 | 57.174 | 3.596 | 9.112 | 1.00 | 24.73 | N |
| ATOM | 588 | CZ | ARG | A | 113 | 57.940 | 4.683 | 9.127 | 1.00 | 27.38 | C |
| ATOM | 589 | NH1 | ARG | A | 113 | 59.186 | 4.641 | 8.653 | 1.00 | 22.36 | N |
| ATOM | 590 | NH2 | ARG | A | 113 | 57.454 | 5.821 | 9.607 | 1.00 | 26.74 | N |
| ATOM | 591 | N | SER | A | 114 | 55.644 | 0.728 | 4.354 | 1.00 | 19.79 | N |
| ATOM | 592 | CA | SER | A | 114 | 56.238 | −0.239 | 3.428 | 1.00 | 22.49 | C |
| ATOM | 593 | C | SER | A | 114 | 55.263 | −1.193 | 2.748 | 1.00 | 22.79 | C |
| ATOM | 594 | O | SER | A | 114 | 55.674 | −2.212 | 2.191 | 1.00 | 21.77 | O |
| ATOM | 595 | CB | SER | A | 114 | 57.317 | −1.053 | 4.140 | 1.00 | 23.99 | C |
| ATOM | 596 | OG | SER | A | 114 | 58.308 | −0.194 | 4.663 | 1.00 | 28.32 | O |
| ATOM | 597 | N | HIS | A | 115 | 53.976 | −0.865 | 2.788 | 1.00 | 17.94 | N |
| ATOM | 598 | CA | HIS | A | 115 | 52.965 | −1.705 | 2.157 | 1.00 | 16.51 | C |
| ATOM | 599 | C | HIS | A | 115 | 52.122 | −0.874 | 1.205 | 1.00 | 17.14 | C |
| ATOM | 600 | O | HIS | A | 115 | 52.030 | 0.350 | 1.347 | 1.00 | 21.41 | O |
| ATOM | 601 | CB | HIS | A | 115 | 52.046 | −2.341 | 3.208 | 1.00 | 16.90 | C |
| ATOM | 602 | CG | HIS | A | 115 | 52.704 | −3.405 | 4.032 | 1.00 | 14.40 | C |
| ATOM | 603 | ND1 | HIS | A | 115 | 53.636 | −3.123 | 5.007 | 1.00 | 14.85 | N |
| ATOM | 604 | CD2 | HIS | A | 115 | 52.566 | −4.752 | 4.021 | 1.00 | 12.32 | C |
| ATOM | 605 | CE1 | HIS | A | 115 | 54.043 | −4.250 | 5.564 | 1.00 | 15.05 | C |
| ATOM | 606 | NE2 | HIS | A | 115 | 53.409 | −5.253 | 4.984 | 1.00 | 15.51 | N |
| ATOM | 607 | N | GLY | A | 116 | 51.501 | −1.543 | 0.241 | 1.00 | 16.83 | N |
| ATOM | 608 | CA | GLY | A | 116 | 50.654 | −0.848 | −0.713 | 1.00 | 18.77 | C |
| ATOM | 609 | C | GLY | A | 116 | 51.298 | 0.369 | −1.354 | 1.00 | 17.87 | C |
| ATOM | 610 | O | GLY | A | 116 | 52.442 | 0.311 | −1.801 | 1.00 | 17.79 | O |
| ATOM | 611 | N | GLU | A | 117 | 50.564 | 1.480 | −1.382 | 1.00 | 20.44 | N |
| ATOM | 612 | CA | GLU | A | 117 | 51.049 | 2.714 | −1.990 | 1.00 | 19.63 | C |
| ATOM | 613 | C | GLU | A | 117 | 51.633 | 3.725 | −1.003 | 1.00 | 20.15 | C |
| ATOM | 614 | O | GLU | A | 117 | 51.900 | 4.869 | −1.367 | 1.00 | 18.55 | O |
| ATOM | 615 | CB | GLU | A | 117 | 49.917 | 3.383 | −2.777 | 1.00 | 23.47 | C |
| ATOM | 616 | CG | GLU | A | 117 | 49.407 | 2.594 | −3.983 | 1.00 | 29.04 | C |
| ATOM | 617 | CD | GLU | A | 117 | 50.449 | 2.444 | −5.079 | 1.00 | 34.14 | C |
| ATOM | 618 | OE1 | GLU | A | 117 | 51.240 | 3.386 | −5.286 | 1.00 | 31.01 | O |
| ATOM | 619 | OE2 | GLU | A | 117 | 50.469 | 1.388 | −5.747 | 1.00 | 38.27 | O |
| ATOM | 620 | N | THR | A | 118 | 51.833 | 3.315 | 0.244 | 1.00 | 19.24 | N |
| ATOM | 621 | CA | THR | A | 118 | 52.393 | 4.220 | 1.245 | 1.00 | 19.66 | C |
| ATOM | 622 | C | THR | A | 118 | 53.891 | 4.413 | 1.003 | 1.00 | 22.22 | C |
| ATOM | 623 | O | THR | A | 118 | 54.603 | 3.459 | 0.697 | 1.00 | 21.02 | O |
| ATOM | 624 | CB | THR | A | 118 | 52.127 | 3.670 | 2.658 | 1.00 | 18.29 | C |
| ATOM | 625 | OG1 | THR | A | 118 | 50.774 | 3.983 | 3.027 | 1.00 | 16.35 | O |
| ATOM | 626 | CG2 | THR | A | 118 | 53.093 | 4.246 | 3.664 | 1.00 | 15.97 | C |
| ATOM | 627 | N | LYS | A | 119 | 54.358 | 5.654 | 1.131 | 1.00 | 21.87 | N |
| ATOM | 628 | CA | LYS | A | 119 | 55.766 | 5.982 | 0.898 | 1.00 | 22.93 | C |
| ATOM | 629 | C | LYS | A | 119 | 56.406 | 6.679 | 2.098 | 1.00 | 23.46 | C |
| ATOM | 630 | O | LYS | A | 119 | 56.120 | 7.850 | 2.362 | 1.00 | 21.76 | O |
| ATOM | 631 | CB | LYS | A | 119 | 55.892 | 6.906 | −0.317 | 1.00 | 25.48 | C |
| ATOM | 632 | CG | LYS | A | 119 | 55.142 | 6.437 | −1.551 | 1.00 | 29.17 | C |
| ATOM | 633 | CD | LYS | A | 119 | 55.686 | 5.109 | −2.056 | 1.00 | 32.86 | C |
| ATOM | 634 | CE | LYS | A | 119 | 54.934 | 4.646 | −3.296 | 1.00 | 36.54 | C |
| ATOM | 635 | NZ | LYS | A | 119 | 55.342 | 3.284 | −3.725 | 1.00 | 36.39 | N |
| ATOM | 636 | N | VAL | A | 120 | 57.270 | 5.966 | 2.816 | 1.00 | 22.14 | N |
| ATOM | 637 | CA | VAL | A | 120 | 57.955 | 6.535 | 3.976 | 1.00 | 23.27 | C |
| ATOM | 638 | C | VAL | A | 120 | 59.443 | 6.694 | 3.671 | 1.00 | 26.40 | C |
| ATOM | 639 | O | VAL | A | 120 | 59.975 | 6.046 | 2.765 | 1.00 | 25.34 | O |
| ATOM | 640 | CB | VAL | A | 120 | 57.808 | 5.636 | 5.221 | 1.00 | 23.46 | C |
| ATOM | 641 | CG1 | VAL | A | 120 | 56.331 | 5.396 | 5.513 | 1.00 | 19.68 | C |
| ATOM | 642 | CG2 | VAL | A | 120 | 58.546 | 4.315 | 5.002 | 1.00 | 25.86 | C |
| ATOM | 643 | N | LYS | A | 121 | 60.122 | 7.545 | 4.432 | 1.00 | 27.85 | N |
| ATOM | 644 | CA | LYS | A | 121 | 61.543 | 7.764 | 4.199 | 1.00 | 33.03 | C |
| ATOM | 645 | C | LYS | A | 121 | 62.390 | 6.596 | 4.712 | 1.00 | 32.36 | C |
| ATOM | 646 | O | LYS | A | 121 | 63.433 | 6.290 | 4.140 | 1.00 | 33.67 | O |
| ATOM | 647 | CB | LYS | A | 121 | 61.988 | 9.082 | 4.850 | 1.00 | 37.98 | C |
| ATOM | 648 | CG | LYS | A | 121 | 62.711 | 8.933 | 6.180 | 1.00 | 46.79 | C |
| ATOM | 649 | CD | LYS | A | 121 | 64.188 | 8.580 | 5.984 | 1.00 | 54.25 | C |
| ATOM | 650 | CE | LYS | A | 121 | 64.881 | 8.289 | 7.311 | 1.00 | 56.45 | C |
| ATOM | 651 | NZ | LYS | A | 121 | 66.333 | 7.999 | 7.127 | 1.00 | 60.59 | N |
| ATOM | 652 | N | ASN | A | 122 | 61.944 | 5.942 | 5.781 | 1.00 | 31.59 | N |
| ATOM | 653 | CA | ASN | A | 122 | 62.689 | 4.818 | 6.339 | 1.00 | 32.03 | C |
| ATOM | 654 | C | ASN | A | 122 | 61.822 | 3.563 | 6.347 | 1.00 | 30.59 | C |
| ATOM | 655 | O | ASN | A | 122 | 61.335 | 3.129 | 7.391 | 1.00 | 27.09 | O |
| ATOM | 656 | CB | ASN | A | 122 | 63.158 | 5.154 | 7.753 | 1.00 | 35.19 | C |
| ATOM | 657 | CG | ASN | A | 122 | 64.031 | 4.074 | 8.346 | 1.00 | 41.23 | C |
| ATOM | 658 | OD1 | ASN | A | 122 | 64.555 | 3.215 | 7.628 | 1.00 | 45.50 | O |
| ATOM | 659 | ND2 | ASN | A | 122 | 64.208 | 4.114 | 9.665 | 1.00 | 41.49 | N |
| ATOM | 660 | N | PRO | A | 123 | 61.638 | 2.953 | 5.170 | 1.00 | 28.38 | N |
| ATOM | 661 | CA | PRO | A | 123 | 60.831 | 1.746 | 4.971 | 1.00 | 29.19 | C |
| ATOM | 662 | C | PRO | A | 123 | 61.313 | 0.473 | 5.661 | 1.00 | 29.16 | C |
| ATOM | 663 | O | PRO | A | 123 | 60.591 | −0.522 | 5.700 | 1.00 | 29.24 | O |
| ATOM | 664 | CB | PRO | A | 123 | 60.808 | 1.609 | 3.454 | 1.00 | 29.53 | C |
| ATOM | 665 | CG | PRO | A | 123 | 62.161 | 2.119 | 3.069 | 1.00 | 30.05 | C |
| ATOM | 666 | CD | PRO | A | 123 | 62.275 | 3.365 | 3.906 | 1.00 | 29.80 | C |
| ATOM | 667 | N | GLU | A | 124 | 62.520 | 0.500 | 6.214 | 1.00 | 29.09 | N |
| ATOM | 668 | CA | GLU | A | 124 | 63.063 | −0.678 | 6.882 | 1.00 | 29.07 | C |
| ATOM | 669 | C | GLU | A | 124 | 62.557 | −0.822 | 8.316 | 1.00 | 28.09 | C |
| ATOM | 670 | O | GLU | A | 124 | 62.476 | −1.932 | 8.847 | 1.00 | 23.75 | O |
| ATOM | 671 | CB | GLU | A | 124 | 64.592 | −0.615 | 6.872 | 1.00 | 32.56 | C |
| ATOM | 672 | CG | GLU | A | 124 | 65.296 | −1.837 | 7.451 | 1.00 | 44.00 | C |
| ATOM | 673 | CD | GLU | A | 124 | 65.257 | −1.886 | 8.970 | 1.00 | 48.21 | C |
| ATOM | 674 | OE1 | GLU | A | 124 | 65.545 | −0.846 | 9.600 | 1.00 | 51.97 | O |
| ATOM | 675 | OE2 | GLU | A | 124 | 64.950 | −2.962 | 9.531 | 1.00 | 48.81 | O |
| ATOM | 676 | N | ASP | A | 125 | 62.201 | 0.300 | 8.928 | 1.00 | 24.64 | N |
| ATOM | 677 | CA | ASP | A | 125 | 61.728 | 0.313 | 10.309 | 1.00 | 25.01 | C |
| ATOM | 678 | C | ASP | A | 125 | 60.267 | −0.108 | 10.496 | 1.00 | 24.87 | C |
| ATOM | 679 | O | ASP | A | 125 | 59.362 | 0.721 | 10.447 | 1.00 | 23.97 | O |
| ATOM | 680 | CB | ASP | A | 125 | 61.950 | 1.710 | 10.901 | 1.00 | 22.77 | C |
| ATOM | 681 | CG | ASP | A | 125 | 61.418 | 1.846 | 12.316 | 1.00 | 25.58 | C |
| ATOM | 682 | OD1 | ASP | A | 125 | 61.176 | 0.816 | 12.982 | 1.00 | 26.75 | O |
| ATOM | 683 | OD2 | ASP | A | 125 | 61.256 | 2.996 | 12.770 | 1.00 | 26.71 | O |
| ATOM | 684 | N | LEU | A | 126 | 60.038 | −1.400 | 10.716 | 1.00 | 22.44 | N |
| ATOM | 685 | CA | LEU | A | 126 | 58.679 | −1.883 | 10.938 | 1.00 | 22.87 | C |
| ATOM | 686 | C | LEU | A | 126 | 58.451 | −2.289 | 12.395 | 1.00 | 23.25 | C |
| ATOM | 687 | O | LEU | A | 126 | 57.716 | −3.237 | 12.685 | 1.00 | 21.64 | O |
| ATOM | 688 | CB | LEU | A | 126 | 58.365 | −3.052 | 10.002 | 1.00 | 27.05 | C |
| ATOM | 689 | CG | LEU | A | 126 | 57.572 | −2.671 | 8.741 | 1.00 | 33.52 | C |
| ATOM | 690 | CD1 | LEU | A | 126 | 58.334 | −1.635 | 7.944 | 1.00 | 35.30 | C |
| ATOM | 691 | CD2 | LEU | A | 126 | 57.312 | −3.905 | 7.893 | 1.00 | 32.86 | C |
| ATOM | 692 | N | SER | A | 127 | 59.080 | −1.562 | 13.313 | 1.00 | 20.54 | N |
| ATOM | 693 | CA | SER | A | 127 | 58.927 | −1.835 | 14.738 | 1.00 | 20.80 | C |
| ATOM | 694 | C | SER | A | 127 | 57.490 | −1.499 | 15.142 | 1.00 | 23.21 | C |
| ATOM | 695 | O | SER | A | 127 | 56.861 | −0.624 | 14.544 | 1.00 | 19.57 | O |
| ATOM | 696 | CB | SER | A | 127 | 59.901 | −0.974 | 15.550 | 1.00 | 21.93 | C |
| ATOM | 697 | OG | SER | A | 127 | 59.617 | 0.406 | 15.386 | 1.00 | 21.08 | O |
| ATOM | 698 | N | ALA | A | 128 | 56.967 | −2.193 | 16.146 | 1.00 | 21.78 | N |
| ATOM | 699 | CA | ALA | A | 128 | 55.604 | −1.935 | 16.597 | 1.00 | 22.51 | C |
| ATOM | 700 | C | ALA | A | 128 | 55.463 | −0.455 | 16.950 | 1.00 | 22.66 | C |
| ATOM | 701 | O | ALA | A | 128 | 54.488 | 0.203 | 16.590 | 1.00 | 18.71 | O |
| ATOM | 702 | CB | ALA | A | 128 | 55.283 | −2.792 | 17.817 | 1.00 | 22.86 | C |
| ATOM | 703 | N | GLU | A | 129 | 56.461 | 0.056 | 17.655 | 1.00 | 21.49 | N |
| ATOM | 704 | CA | GLU | A | 129 | 56.485 | 1.443 | 18.089 | 1.00 | 23.38 | C |
| ATOM | 705 | C | GLU | A | 129 | 56.285 | 2.427 | 16.930 | 1.00 | 21.47 | C |
| ATOM | 706 | O | GLU | A | 129 | 55.415 | 3.299 | 16.971 | 1.00 | 19.37 | O |
| ATOM | 707 | CB | GLU | A | 129 | 57.821 | 1.731 | 18.778 | 1.00 | 30.80 | C |
| ATOM | 708 | CG | GLU | A | 129 | 58.085 | 0.940 | 20.072 | 1.00 | 36.96 | C |
| ATOM | 709 | CD | GLU | A | 129 | 58.038 | −0.586 | 19.907 | 1.00 | 42.22 | C |
| ATOM | 710 | OE1 | GLU | A | 129 | 58.703 | −1.137 | 18.990 | 1.00 | 28.12 | O |
| ATOM | 711 | OE2 | GLU | A | 129 | 57.335 | −1.236 | 20.720 | 1.00 | 41.15 | O |
| ATOM | 712 | N | THR | A | 130 | 57.097 | 2.284 | 15.894 | 1.00 | 20.54 | N |
| ATOM | 713 | CA | THR | A | 130 | 57.017 | 3.177 | 14.748 | 1.00 | 20.81 | C |
| ATOM | 714 | C | THR | A | 130 | 55.697 | 3.056 | 13.994 | 1.00 | 19.05 | C |
| ATOM | 715 | O | THR | A | 130 | 55.078 | 4.063 | 13.652 | 1.00 | 18.54 | O |
| ATOM | 716 | CB | THR | A | 130 | 58.201 | 2.920 | 13.786 | 1.00 | 22.03 | C |
| ATOM | 717 | OG1 | THR | A | 130 | 59.424 | 3.238 | 14.459 | 1.00 | 22.20 | O |
| ATOM | 718 | CG2 | THR | A | 130 | 58.083 | 3.779 | 12.536 | 1.00 | 23.68 | C |
| ATOM | 719 | N | MET | A | 131 | 55.261 | 1.824 | 13.750 | 1.00 | 15.95 | N |
| ATOM | 720 | CA | MET | A | 131 | 54.017 | 1.594 | 13.031 | 1.00 | 16.89 | C |
| ATOM | 721 | C | MET | A | 131 | 52.807 | 2.137 | 13.794 | 1.00 | 17.15 | C |
| ATOM | 722 | O | MET | A | 131 | 51.900 | 2.707 | 13.190 | 1.00 | 18.70 | O |
| ATOM | 723 | CB | MET | A | 131 | 53.845 | 0.095 | 12.742 | 1.00 | 18.80 | C |
| ATOM | 724 | CG | MET | A | 131 | 54.930 | −0.474 | 11.810 | 1.00 | 19.73 | C |
| ATOM | 725 | SD | MET | A | 131 | 54.659 | −2.213 | 11.356 | 1.00 | 23.66 | S |
| ATOM | 726 | CE | MET | A | 131 | 53.448 | −2.010 | 10.072 | 1.00 | 13.32 | C |
| ATOM | 727 | N | ALA | A | 132 | 52.787 | 1.970 | 15.115 | 1.00 | 16.11 | N |
| ATOM | 728 | CA | ALA | A | 132 | 51.665 | 2.480 | 15.902 | 1.00 | 17.86 | C |
| ATOM | 729 | C | ALA | A | 132 | 51.662 | 4.008 | 15.815 | 1.00 | 17.53 | C |
| ATOM | 730 | O | ALA | A | 132 | 50.613 | 4.632 | 15.674 | 1.00 | 17.64 | O |
| ATOM | 731 | CB | ALA | A | 132 | 51.786 | 2.031 | 17.357 | 1.00 | 18.40 | C |
| ATOM | 732 | N | LYS | A | 133 | 52.851 | 4.597 | 15.893 | 1.00 | 16.62 | N |
| ATOM | 733 | CA | LYS | A | 133 | 53.014 | 6.047 | 15.805 | 1.00 | 16.89 | C |
| ATOM | 734 | C | LYS | A | 133 | 52.458 | 6.533 | 14.458 | 1.00 | 16.16 | C |
| ATOM | 735 | O | LYS | A | 133 | 51.772 | 7.561 | 14.392 | 1.00 | 15.82 | O |
| ATOM | 736 | CB | LYS | A | 133 | 54.502 | 6.401 | 15.951 | 1.00 | 17.44 | C |
| ATOM | 737 | CG | LYS | A | 133 | 54.863 | 7.868 | 15.736 | 1.00 | 30.01 | C |
| ATOM | 738 | CD | LYS | A | 133 | 54.884 | 8.674 | 17.034 | 1.00 | 34.29 | C |
| ATOM | 739 | CE | LYS | A | 133 | 53.491 | 8.820 | 17.626 | 1.00 | 39.05 | C |
| ATOM | 740 | NZ | LYS | A | 133 | 53.394 | 9.769 | 18.777 | 1.00 | 33.22 | N |
| ATOM | 741 | N | ASP | A | 134 | 52.737 | 5.783 | 13.392 | 1.00 | 14.44 | N |
| ATOM | 742 | CA | ASP | A | 134 | 52.235 | 6.125 | 12.061 | 1.00 | 16.17 | C |
| ATOM | 743 | C | ASP | A | 134 | 50.710 | 6.233 | 12.077 | 1.00 | 16.87 | C |
| ATOM | 744 | O | ASP | A | 134 | 50.138 | 7.207 | 11.582 | 1.00 | 16.15 | O |
| ATOM | 745 | CB | ASP | A | 134 | 52.629 | 5.061 | 11.021 | 1.00 | 19.21 | C |
| ATOM | 746 | CG | ASP | A | 134 | 54.121 | 5.061 | 10.696 | 1.00 | 21.28 | C |
| ATOM | 747 | OD1 | ASP | A | 134 | 54.786 | 6.099 | 10.900 | 1.00 | 20.40 | O |
| ATOM | 748 | OD2 | ASP | A | 134 | 54.625 | 4.018 | 10.216 | 1.00 | 20.37 | O |
| ATOM | 749 | N | VAL | A | 135 | 50.051 | 5.215 | 12.625 | 1.00 | 13.61 | N |
| ATOM | 750 | CA | VAL | A | 135 | 48.595 | 5.209 | 12.694 | 1.00 | 13.99 | C |
| ATOM | 751 | C | VAL | A | 135 | 48.090 | 6.470 | 13.416 | 1.00 | 15.22 | C |
| ATOM | 752 | O | VAL | A | 135 | 47.180 | 7.145 | 12.937 | 1.00 | 15.49 | O |
| ATOM | 753 | CB | VAL | A | 135 | 48.086 | 3.946 | 13.425 | 1.00 | 16.07 | C |
| ATOM | 754 | CG1 | VAL | A | 135 | 46.555 | 3.964 | 13.515 | 1.00 | 12.82 | C |
| ATOM | 755 | CG2 | VAL | A | 135 | 48.555 | 2.695 | 12.671 | 1.00 | 13.26 | C |
| ATOM | 756 | N | GLY | A | 136 | 48.687 | 6.785 | 14.560 | 1.00 | 15.02 | N |
| ATOM | 757 | CA | GLY | A | 136 | 48.271 | 7.970 | 15.296 | 1.00 | 18.63 | C |
| ATOM | 758 | C | GLY | A | 136 | 48.496 | 9.232 | 14.476 | 1.00 | 16.49 | C |
| ATOM | 759 | O | GLY | A | 136 | 47.634 | 10.116 | 14.416 | 1.00 | 17.55 | O |
| ATOM | 760 | N | ASN | A | 137 | 49.660 | 9.316 | 13.840 | 1.00 | 16.58 | N |
| ATOM | 761 | CA | ASN | A | 137 | 50.003 | 10.470 | 13.012 | 1.00 | 19.78 | C |
| ATOM | 762 | C | ASN | A | 137 | 49.031 | 10.634 | 11.854 | 1.00 | 17.81 | C |
| ATOM | 763 | O | ASN | A | 137 | 48.665 | 11.749 | 11.492 | 1.00 | 17.37 | O |
| ATOM | 764 | CB | ASN | A | 137 | 51.420 | 10.332 | 12.445 | 1.00 | 19.08 | C |
| ATOM | 765 | CG | ASN | A | 137 | 52.490 | 10.526 | 13.493 | 1.00 | 22.27 | C |
| ATOM | 766 | OD1 | ASN | A | 137 | 52.229 | 11.049 | 14.574 | 1.00 | 22.31 | O |
| ATOM | 767 | ND2 | ASN | A | 137 | 53.713 | 10.120 | 13.170 | 1.00 | 18.38 | N |
| ATOM | 768 | N | VAL | A | 138 | 48.620 | 9.519 | 11.260 | 1.00 | 16.34 | N |
| ATOM | 769 | CA | VAL | A | 138 | 47.684 | 9.581 | 10.148 | 1.00 | 16.39 | C |
| ATOM | 770 | C | VAL | A | 138 | 46.335 | 10.141 | 10.602 | 1.00 | 15.96 | C |
| ATOM | 771 | O | VAL | A | 138 | 45.774 | 11.018 | 9.948 | 1.00 | 16.42 | O |
| ATOM | 772 | CB | VAL | A | 138 | 47.480 | 8.191 | 9.505 | 1.00 | 16.09 | C |
| ATOM | 773 | CG1 | VAL | A | 138 | 46.263 | 8.207 | 8.591 | 1.00 | 14.79 | C |
| ATOM | 774 | CG2 | VAL | A | 138 | 48.726 | 7.811 | 8.703 | 1.00 | 15.29 | C |
| ATOM | 775 | N | VAL | A | 139 | 45.816 | 9.636 | 11.717 | 1.00 | 15.07 | N |
| ATOM | 776 | CA | VAL | A | 139 | 44.531 | 10.116 | 12.234 | 1.00 | 13.85 | C |
| ATOM | 777 | C | VAL | A | 139 | 44.623 | 11.610 | 12.544 | 1.00 | 16.36 | C |
| ATOM | 778 | O | VAL | A | 139 | 43.698 | 12.375 | 12.268 | 1.00 | 16.20 | O |
| ATOM | 779 | CB | VAL | A | 139 | 44.130 | 9.354 | 13.520 | 1.00 | 14.99 | C |
| ATOM | 780 | CG1 | VAL | A | 139 | 42.916 | 10.023 | 14.185 | 1.00 | 15.39 | C |
| ATOM | 781 | CG2 | VAL | A | 139 | 43.796 | 7.898 | 13.171 | 1.00 | 17.30 | C |
| ATOM | 782 | N | GLU | A | 140 | 45.748 | 12.019 | 13.117 | 1.00 | 16.03 | N |
| ATOM | 783 | CA | GLU | A | 140 | 45.960 | 13.417 | 13.459 | 1.00 | 18.64 | C |
| ATOM | 784 | C | GLU | A | 140 | 45.943 | 14.293 | 12.209 | 1.00 | 19.28 | C |
| ATOM | 785 | O | GLU | A | 140 | 45.319 | 15.352 | 12.187 | 1.00 | 19.34 | O |
| ATOM | 786 | CB | GLU | A | 140 | 47.296 | 13.571 | 14.186 | 1.00 | 21.19 | C |
| ATOM | 787 | CG | GLU | A | 140 | 47.652 | 14.999 | 14.539 | 1.00 | 31.19 | C |
| ATOM | 788 | CD | GLU | A | 140 | 48.831 | 15.074 | 15.486 | 1.00 | 40.04 | C |
| ATOM | 789 | OE1 | GLU | A | 140 | 49.876 | 14.440 | 15.201 | 1.00 | 46.79 | O |
| ATOM | 790 | OE2 | GLU | A | 140 | 48.716 | 15.771 | 16.515 | 1.00 | 45.39 | O |
| ATOM | 791 | N | ALA | A | 141 | 46.626 | 13.843 | 11.163 | 1.00 | 18.52 | N |
| ATOM | 792 | CA | ALA | A | 141 | 46.684 | 14.603 | 9.919 | 1.00 | 18.24 | C |
| ATOM | 793 | C | ALA | A | 141 | 45.345 | 14.646 | 9.195 | 1.00 | 18.01 | C |
| ATOM | 794 | O | ALA | A | 141 | 44.984 | 15.658 | 8.600 | 1.00 | 15.85 | O |
| ATOM | 795 | CB | ALA | A | 141 | 47.748 | 14.017 | 9.002 | 1.00 | 17.64 | C |
| ATOM | 796 | N | MET | A | 142 | 44.602 | 13.549 | 9.247 | 1.00 | 17.45 | N |
| ATOM | 797 | CA | MET | A | 142 | 43.317 | 13.480 | 8.565 | 1.00 | 17.77 | C |
| ATOM | 798 | C | MET | A | 142 | 42.207 | 14.314 | 9.180 | 1.00 | 18.24 | C |
| ATOM | 799 | O | MET | A | 142 | 41.384 | 14.872 | 8.457 | 1.00 | 16.13 | O |
| ATOM | 800 | CB | MET | A | 142 | 42.820 | 12.031 | 8.498 | 1.00 | 18.63 | C |
| ATOM | 801 | CG | MET | A | 142 | 43.577 | 11.129 | 7.536 | 1.00 | 16.24 | C |
| ATOM | 802 | SD | MET | A | 142 | 42.836 | 9.485 | 7.504 | 1.00 | 18.13 | S |
| ATOM | 803 | CE | MET | A | 142 | 41.300 | 9.835 | 6.693 | 1.00 | 20.76 | C |
| ATOM | 804 | N | TYR | A | 143 | 42.181 | 14.403 | 10.507 | 1.00 | 16.78 | N |
| ATOM | 805 | CA | TYR | A | 143 | 41.103 | 15.118 | 11.189 | 1.00 | 20.30 | C |
| ATOM | 806 | C | TYR | A | 143 | 41.507 | 16.249 | 12.122 | 1.00 | 19.68 | C |
| ATOM | 807 | O | TYR | A | 143 | 40.645 | 16.967 | 12.630 | 1.00 | 19.22 | O |
| ATOM | 808 | CB | TYR | A | 143 | 40.271 | 14.120 | 11.995 | 1.00 | 19.03 | C |
| ATOM | 809 | CG | TYR | A | 143 | 39.901 | 12.866 | 11.240 | 1.00 | 17.81 | C |
| ATOM | 810 | CD1 | TYR | A | 143 | 38.971 | 12.900 | 10.202 | 1.00 | 19.04 | C |
| ATOM | 811 | CD2 | TYR | A | 143 | 40.447 | 11.636 | 11.596 | 1.00 | 17.85 | C |
| ATOM | 812 | CE1 | TYR | A | 143 | 38.587 | 11.732 | 9.542 | 1.00 | 18.53 | C |
| ATOM | 813 | CE2 | TYR | A | 143 | 40.072 | 10.465 | 10.943 | 1.00 | 17.78 | C |
| ATOM | 814 | CZ | TYR | A | 143 | 39.141 | 10.520 | 9.920 | 1.00 | 16.45 | C |
| ATOM | 815 | OH | TYR | A | 143 | 38.756 | 9.361 | 9.285 | 1.00 | 18.91 | O |
| ATOM | 816 | N | GLY | A | 144 | 42.801 | 16.395 | 12.370 | 1.00 | 17.72 | N |
| ATOM | 817 | CA | GLY | A | 144 | 43.250 | 17.450 | 13.255 | 1.00 | 24.51 | C |
| ATOM | 818 | C | GLY | A | 144 | 42.694 | 17.306 | 14.659 | 1.00 | 29.99 | C |
| ATOM | 819 | O | GLY | A | 144 | 42.811 | 16.246 | 15.276 | 1.00 | 29.28 | O |
| ATOM | 820 | N | ASP | A | 145 | 42.067 | 18.371 | 15.155 | 1.00 | 33.36 | N |
| ATOM | 821 | CA | ASP | A | 145 | 41.508 | 18.380 | 16.505 | 1.00 | 36.16 | C |
| ATOM | 822 | C | ASP | A | 145 | 40.063 | 17.897 | 16.597 | 1.00 | 34.84 | C |
| ATOM | 823 | O | ASP | A | 145 | 39.500 | 17.834 | 17.687 | 1.00 | 32.16 | O |
| ATOM | 824 | CB | ASP | A | 145 | 41.601 | 19.790 | 17.104 | 1.00 | 40.41 | C |
| ATOM | 825 | CG | ASP | A | 145 | 43.033 | 20.282 | 17.221 | 1.00 | 45.06 | C |
| ATOM | 826 | OD1 | ASP | A | 145 | 43.834 | 19.630 | 17.928 | 1.00 | 47.90 | O |
| ATOM | 827 | OD2 | ASP | A | 145 | 43.356 | 21.322 | 16.605 | 1.00 | 45.98 | O |
| ATOM | 828 | N | LEU | A | 146 | 39.455 | 17.557 | 15.467 | 1.00 | 30.84 | N |
| ATOM | 829 | CA | LEU | A | 146 | 38.075 | 17.093 | 15.497 | 1.00 | 29.36 | C |
| ATOM | 830 | C | LEU | A | 146 | 37.910 | 15.724 | 14.831 | 1.00 | 26.68 | C |
| ATOM | 831 | O | LEU | A | 146 | 37.309 | 15.604 | 13.755 | 1.00 | 25.52 | O |
| ATOM | 832 | CB | LEU | A | 146 | 37.173 | 18.135 | 14.825 | 1.00 | 31.55 | C |
| ATOM | 833 | CG | LEU | A | 146 | 35.747 | 18.245 | 15.372 | 1.00 | 36.16 | C |
| ATOM | 834 | CD1 | LEU | A | 146 | 35.103 | 19.542 | 14.868 | 1.00 | 32.10 | C |
| ATOM | 835 | CD2 | LEU | A | 146 | 34.938 | 17.023 | 14.960 | 1.00 | 35.55 | C |
| ATOM | 836 | N | PRO | A | 147 | 38.450 | 14.668 | 15.463 | 1.00 | 24.51 | N |
| ATOM | 837 | CA | PRO | A | 147 | 38.338 | 13.321 | 14.895 | 1.00 | 22.06 | C |
| ATOM | 838 | C | PRO | A | 147 | 36.995 | 12.668 | 15.192 | 1.00 | 21.66 | C |
| ATOM | 839 | O | PRO | A | 147 | 36.375 | 12.928 | 16.227 | 1.00 | 21.03 | O |
| ATOM | 840 | CB | PRO | A | 147 | 39.481 | 12.574 | 15.566 | 1.00 | 22.16 | C |
| ATOM | 841 | CG | PRO | A | 147 | 39.474 | 13.173 | 16.948 | 1.00 | 23.75 | C |
| ATOM | 842 | CD | PRO | A | 147 | 39.312 | 14.664 | 16.660 | 1.00 | 22.86 | C |
| ATOM | 843 | N | PRO | A | 148 | 36.523 | 11.813 | 14.281 | 1.00 | 20.97 | N |
| ATOM | 844 | CA | PRO | A | 148 | 35.244 | 11.123 | 14.483 | 1.00 | 18.56 | C |
| ATOM | 845 | C | PRO | A | 148 | 35.478 | 9.938 | 15.430 | 1.00 | 17.46 | C |
| ATOM | 846 | O | PRO | A | 148 | 36.627 | 9.600 | 15.736 | 1.00 | 17.12 | O |
| ATOM | 847 | CB | PRO | A | 148 | 34.896 | 10.653 | 13.079 | 1.00 | 20.13 | C |
| ATOM | 848 | CG | PRO | A | 148 | 36.251 | 10.307 | 12.523 | 1.00 | 25.63 | C |
| ATOM | 849 | CD | PRO | A | 148 | 37.109 | 11.471 | 12.971 | 1.00 | 21.17 | C |
| ATOM | 850 | N | PRO | A | 149 | 34.399 | 9.313 | 15.928 | 1.00 | 14.78 | N |
| ATOM | 851 | CA | PRO | A | 149 | 34.598 | 8.167 | 16.824 | 1.00 | 14.97 | C |
| ATOM | 852 | C | PRO | A | 149 | 35.295 | 7.115 | 15.958 | 1.00 | 16.48 | C |
| ATOM | 853 | O | PRO | A | 149 | 34.955 | 6.960 | 14.781 | 1.00 | 17.11 | O |
| ATOM | 854 | CB | PRO | A | 149 | 33.168 | 7.744 | 17.187 | 1.00 | 13.90 | C |
| ATOM | 855 | CG | PRO | A | 149 | 32.354 | 8.995 | 16.956 | 1.00 | 19.14 | C |
| ATOM | 856 | CD | PRO | A | 149 | 32.967 | 9.561 | 15.694 | 1.00 | 15.22 | C |
| ATOM | 857 | N | ILE | A | 150 | 36.253 | 6.392 | 16.526 | 1.00 | 16.75 | N |
| ATOM | 858 | CA | ILE | A | 150 | 36.981 | 5.398 | 15.748 | 1.00 | 16.04 | C |
| ATOM | 859 | C | ILE | A | 150 | 37.056 | 4.017 | 16.385 | 1.00 | 15.36 | C |
| ATOM | 860 | O | ILE | A | 150 | 37.251 | 3.894 | 17.588 | 1.00 | 15.24 | O |
| ATOM | 861 | CB | ILE | A | 150 | 38.433 | 5.868 | 15.495 | 1.00 | 15.32 | C |
| ATOM | 862 | CG1 | ILE | A | 150 | 38.435 | 7.069 | 14.540 | 1.00 | 16.68 | C |
| ATOM | 863 | CG2 | ILE | A | 150 | 39.280 | 4.714 | 14.934 | 1.00 | 17.80 | C |
| ATOM | 864 | CD1 | ILE | A | 150 | 39.802 | 7.718 | 14.381 | 1.00 | 13.54 | C |
| ATOM | 865 | N | MET | A | 151 | 36.885 | 2.984 | 15.566 | 1.00 | 13.65 | N |
| ATOM | 866 | CA | MET | A | 151 | 37.028 | 1.617 | 16.042 | 1.00 | 16.21 | C |
| ATOM | 867 | C | MET | A | 151 | 38.183 | 1.001 | 15.268 | 1.00 | 14.33 | C |
| ATOM | 868 | O | MET | A | 151 | 38.133 | 0.915 | 14.037 | 1.00 | 15.33 | O |
| ATOM | 869 | CB | MET | A | 151 | 35.782 | 0.772 | 15.795 | 1.00 | 17.23 | C |
| ATOM | 870 | CG | MET | A | 151 | 35.974 | −0.650 | 16.349 | 1.00 | 24.70 | C |
| ATOM | 871 | SD | MET | A | 151 | 34.629 | −1.783 | 16.061 | 1.00 | 28.64 | 5 |
| ATOM | 872 | CE | MET | A | 151 | 35.436 | −3.384 | 16.437 | 1.00 | 27.80 | C |
| ATOM | 873 | N | LEU | A | 152 | 39.234 | 0.603 | 15.977 | 1.00 | 12.82 | N |
| ATOM | 874 | CA | LEU | A | 152 | 40.371 | −0.025 | 15.322 | 1.00 | 14.22 | C |
| ATOM | 875 | C | LEU | A | 152 | 40.044 | −1.491 | 15.038 | 1.00 | 16.47 | C |
| ATOM | 876 | O | LEU | A | 152 | 39.460 | −2.177 | 15.880 | 1.00 | 13.94 | O |
| ATOM | 877 | CB | LEU | A | 152 | 41.617 | 0.055 | 16.216 | 1.00 | 15.28 | C |
| ATOM | 878 | CG | LEU | A | 152 | 42.475 | 1.319 | 16.105 | 1.00 | 16.96 | C |
| ATOM | 879 | CD1 | LEU | A | 152 | 43.529 | 1.350 | 17.218 | 1.00 | 13.19 | C |
| ATOM | 880 | CD2 | LEU | A | 152 | 43.138 | 1.338 | 14.730 | 1.00 | 16.02 | C |
| ATOM | 881 | N | ILE | A | 153 | 40.403 | −1.952 | 13.843 | 1.00 | 15.43 | N |
| ATOM | 882 | CA | ILE | A | 153 | 40.196 | −3.346 | 13.440 | 1.00 | 13.29 | C |
| ATOM | 883 | C | ILE | A | 153 | 41.459 | −3.797 | 12.714 | 1.00 | 14.11 | C |
| ATOM | 884 | O | ILE | A | 153 | 41.832 | −3.213 | 11.702 | 1.00 | 14.42 | O |
| ATOM | 885 | CB | ILE | A | 153 | 39.001 | −3.500 | 12.488 | 1.00 | 12.86 | C |
| ATOM | 886 | CG1 | ILE | A | 153 | 37.717 | −3.028 | 13.183 | 1.00 | 11.95 | C |
| ATOM | 887 | CG2 | ILE | A | 153 | 38.856 | −4.959 | 12.063 | 1.00 | 13.54 | C |
| ATOM | 888 | CD1 | ILE | A | 153 | 36.485 | −3.151 | 12.318 | 1.00 | 9.35 | C |
| ATOM | 889 | N | GLY | A | 154 | 42.123 | −4.831 | 13.227 | 1.00 | 14.30 | N |
| ATOM | 890 | CA | GLY | A | 154 | 43.349 | −5.269 | 12.585 | 1.00 | 13.14 | C |
| ATOM | 891 | C | GLY | A | 154 | 43.512 | −6.763 | 12.423 | 1.00 | 14.41 | C |
| ATOM | 892 | O | GLY | A | 154 | 43.078 | −7.543 | 13.268 | 1.00 | 14.96 | O |
| ATOM | 893 | N | HIS | A | 155 | 44.141 | −7.160 | 11.323 | 1.00 | 14.83 | N |
| ATOM | 894 | CA | HIS | A | 155 | 44.382 | −8.569 | 11.053 | 1.00 | 17.51 | C |
| ATOM | 895 | C | HIS | A | 155 | 45.866 | −8.892 | 11.210 | 1.00 | 16.39 | C |
| ATOM | 896 | O | HIS | A | 155 | 46.724 | −8.196 | 10.667 | 1.00 | 16.68 | O |
| ATOM | 897 | CB | HIS | A | 155 | 43.921 | −8.927 | 9.638 | 1.00 | 19.28 | C |
| ATOM | 898 | CG | HIS | A | 155 | 44.306 | −10.308 | 9.216 | 1.00 | 24.58 | C |
| ATOM | 899 | ND1 | HIS | A | 155 | 45.405 | −10.567 | 8.427 | 1.00 | 24.49 | N |
| ATOM | 900 | CD2 | HIS | A | 155 | 43.767 | −11.513 | 9.519 | 1.00 | 25.75 | C |
| ATOM | 901 | CE1 | HIS | A | 155 | 45.527 | −11.872 | 8.262 | 1.00 | 27.15 | C |
| ATOM | 902 | NE2 | HIS | A | 155 | 44.547 | −12.468 | 8.915 | 1.00 | 26.75 | N |
| ATOM | 903 | N | SER | A | 156 | 46.163 | −9.943 | 11.969 | 1.00 | 19.40 | N |
| ATOM | 904 | CA | SER | A | 156 | 47.541 | −10.375 | 12.190 | 1.00 | 21.83 | C |
| ATOM | 905 | C | SER | A | 156 | 48.471 | −9.216 | 12.567 | 1.00 | 19.27 | C |
| ATOM | 906 | O | SER | A | 156 | 48.282 | −8.592 | 13.605 | 1.00 | 21.48 | O |
| ATOM | 907 | CB | SER | A | 156 | 48.061 | −11.093 | 10.939 | 1.00 | 23.54 | C |
| ATOM | 908 | OG | SER | A | 156 | 49.302 | −11.729 | 11.203 | 1.00 | 31.79 | O |
| ATOM | 909 | N | MET | A | 157 | 49.478 | −8.937 | 11.739 | 1.00 | 18.33 | N |
| ATOM | 910 | CA | MET | A | 157 | 50.400 | −7.837 | 12.024 | 1.00 | 15.43 | C |
| ATOM | 911 | C | MET | A | 157 | 49.596 | −6.569 | 12.335 | 1.00 | 16.03 | C |
| ATOM | 912 | O | MET | A | 157 | 49.931 | −5.824 | 13.254 | 1.00 | 17.36 | O |
| ATOM | 913 | CB | MET | A | 157 | 51.328 | −7.573 | 10.830 | 1.00 | 14.89 | C |
| ATOM | 914 | CG | MET | A | 157 | 52.155 | −6.274 | 10.945 | 1.00 | 15.34 | C |
| ATOM | 915 | SD | MET | A | 157 | 53.063 | −5.816 | 9.430 | 1.00 | 19.33 | 5 |
| ATOM | 916 | CE | MET | A | 157 | 51.724 | −5.130 | 8.415 | 1.00 | 16.62 | C |
| ATOM | 917 | N | GLY | A | 158 | 48.538 | −6.340 | 11.558 | 1.00 | 15.84 | N |
| ATOM | 918 | CA | GLY | A | 158 | 47.691 | −5.174 | 11.757 | 1.00 | 15.07 | C |
| ATOM | 919 | C | GLY | A | 158 | 46.957 | −5.222 | 13.083 | 1.00 | 16.23 | C |
| ATOM | 920 | O | GLY | A | 158 | 46.657 | −4.186 | 13.675 | 1.00 | 14.15 | O |
| ATOM | 921 | N | GLY | A | 159 | 46.651 | −6.429 | 13.545 | 1.00 | 13.52 | N |
| ATOM | 922 | CA | GLY | A | 159 | 45.979 | −6.571 | 14.822 | 1.00 | 14.38 | C |
| ATOM | 923 | C | GLY | A | 159 | 46.933 | −6.160 | 15.932 | 1.00 | 15.49 | C |
| ATOM | 924 | O | GLY | A | 159 | 46.557 | −5.461 | 16.870 | 1.00 | 15.86 | O |
| ATOM | 925 | N | ALA | A | 160 | 48.180 | −6.597 | 15.834 | 1.00 | 14.37 | N |
| ATOM | 926 | CA | ALA | A | 160 | 49.169 | −6.225 | 16.839 | 1.00 | 14.66 | C |
| ATOM | 927 | C | ALA | A | 160 | 49.370 | −4.701 | 16.829 | 1.00 | 14.27 | C |
| ATOM | 928 | O | ALA | A | 160 | 49.432 | −4.069 | 17.885 | 1.00 | 13.28 | O |
| ATOM | 929 | CB | ALA | A | 160 | 50.500 | −6.931 | 16.554 | 1.00 | 15.42 | C |
| ATOM | 930 | N | ILE | A | 161 | 49.470 | −4.111 | 15.638 | 1.00 | 13.65 | N |
| ATOM | 931 | CA | ILE | A | 161 | 49.673 | −2.662 | 15.541 | 1.00 | 15.57 | C |
| ATOM | 932 | C | ILE | A | 161 | 48.466 | −1.934 | 16.116 | 1.00 | 15.43 | C |
| ATOM | 933 | O | ILE | A | 161 | 48.604 | −0.893 | 16.755 | 1.00 | 16.75 | O |
| ATOM | 934 | CB | ILE | A | 161 | 49.899 | −2.200 | 14.076 | 1.00 | 15.47 | C |
| ATOM | 935 | CG1 | ILE | A | 161 | 51.148 | −2.887 | 13.498 | 1.00 | 14.09 | C |
| ATOM | 936 | CG2 | ILE | A | 161 | 50.089 | −0.667 | 14.027 | 1.00 | 13.70 | C |
| ATOM | 937 | CD1 | ILE | A | 161 | 52.398 | −2.783 | 14.397 | 1.00 | 13.26 | C |
| ATOM | 938 | N | ALA | A | 162 | 47.283 | −2.487 | 15.880 | 1.00 | 15.15 | N |
| ATOM | 939 | CA | ALA | A | 162 | 46.056 | −1.900 | 16.403 | 1.00 | 15.44 | C |
| ATOM | 940 | C | ALA | A | 162 | 46.118 | −1.814 | 17.929 | 1.00 | 13.95 | C |
| ATOM | 941 | O | ALA | A | 162 | 45.762 | −0.793 | 18.513 | 1.00 | 15.13 | O |
| ATOM | 942 | CB | ALA | A | 162 | 44.850 | −2.736 | 15.974 | 1.00 | 13.15 | C |
| ATOM | 943 | N | VAL | A | 163 | 46.575 | −2.887 | 18.571 | 1.00 | 13.90 | N |
| ATOM | 944 | CA | VAL | A | 163 | 46.674 | −2.925 | 20.032 | 1.00 | 10.62 | C |
| ATOM | 945 | C | VAL | A | 163 | 47.732 | −1.964 | 20.560 | 1.00 | 12.69 | C |
| ATOM | 946 | O | VAL | A | 163 | 47.509 | −1.262 | 21.548 | 1.00 | 13.53 | O |
| ATOM | 947 | CB | VAL | A | 163 | 47.001 | −4.358 | 20.530 | 1.00 | 16.05 | C |
| ATOM | 948 | CG1 | VAL | A | 163 | 47.184 | −4.365 | 22.043 | 1.00 | 16.70 | C |
| ATOM | 949 | CG2 | VAL | A | 163 | 45.876 | −5.301 | 20.150 | 1.00 | 13.71 | C |
| ATOM | 950 | N | HIS | A | 164 | 48.894 | −1.938 | 19.919 | 1.00 | 12.48 | N |
| ATOM | 951 | CA | HIS | A | 164 | 49.952 | −1.036 | 20.354 | 1.00 | 13.79 | C |
| ATOM | 952 | C | HIS | A | 164 | 49.472 | 0.411 | 20.250 | 1.00 | 15.11 | C |
| ATOM | 953 | O | HIS | A | 164 | 49.797 | 1.240 | 21.089 | 1.00 | 17.01 | O |
| ATOM | 954 | CB | HIS | A | 164 | 51.205 | −1.259 | 19.505 | 1.00 | 15.78 | C |
| ATOM | 955 | CG | HIS | A | 164 | 51.942 | −2.516 | 19.854 | 1.00 | 17.84 | C |
| ATOM | 956 | ND1 | HIS | A | 164 | 52.729 | −2.627 | 20.980 | 1.00 | 16.28 | N |
| ATOM | 957 | CD2 | HIS | A | 164 | 51.972 | −3.729 | 19.253 | 1.00 | 18.37 | C |
| ATOM | 958 | CE1 | HIS | A | 164 | 53.215 | −3.854 | 21.057 | 1.00 | 20.03 | C |
| ATOM | 959 | NE2 | HIS | A | 164 | 52.770 | −4.543 | 20.021 | 1.00 | 16.71 | N |
| ATOM | 960 | N | THR | A | 165 | 48.685 | 0.700 | 19.219 | 1.00 | 14.49 | N |
| ATOM | 961 | CA | THR | A | 165 | 48.138 | 2.040 | 19.006 | 1.00 | 14.57 | C |
| ATOM | 962 | C | THR | A | 165 | 47.139 | 2.402 | 20.122 | 1.00 | 16.33 | C |
| ATOM | 963 | O | THR | A | 165 | 47.240 | 3.458 | 20.751 | 1.00 | 16.52 | O |
| ATOM | 964 | CB | THR | A | 165 | 47.438 | 2.101 | 17.630 | 1.00 | 14.92 | C |
| ATOM | 965 | OG1 | THR | A | 165 | 48.409 | 1.853 | 16.610 | 1.00 | 15.37 | O |
| ATOM | 966 | CG2 | THR | A | 165 | 46.795 | 3.472 | 17.386 | 1.00 | 11.95 | C |
| ATOM | 967 | N | ALA | A | 166 | 46.182 | 1.513 | 20.367 | 1.00 | 13.32 | N |
| ATOM | 968 | CA | ALA | A | 166 | 45.183 | 1.720 | 21.406 | 1.00 | 17.41 | C |
| ATOM | 969 | C | ALA | A | 166 | 45.823 | 1.810 | 22.790 | 1.00 | 20.49 | C |
| ATOM | 970 | O | ALA | A | 166 | 45.462 | 2.661 | 23.607 | 1.00 | 20.53 | O |
| ATOM | 971 | CB | ALA | A | 166 | 44.176 | 0.582 | 21.379 | 1.00 | 14.38 | C |
| ATOM | 972 | N | SER | A | 167 | 46.781 | 0.924 | 23.036 | 1.00 | 20.05 | N |
| ATOM | 973 | CA | SER | A | 167 | 47.480 | 0.857 | 24.307 | 1.00 | 22.75 | C |
| ATOM | 974 | C | SER | A | 167 | 48.263 | 2.131 | 24.618 | 1.00 | 23.77 | C |
| ATOM | 975 | O | SER | A | 167 | 48.328 | 2.563 | 25.767 | 1.00 | 23.48 | O |
| ATOM | 976 | CB | SER | A | 167 | 48.434 | −0.340 | 24.299 | 1.00 | 24.06 | C |
| ATOM | 977 | OG | SER | A | 167 | 49.088 | −0.460 | 25.548 | 1.00 | 35.28 | O |
| ATOM | 978 | N | SER | A | 168 | 48.862 | 2.726 | 23.594 | 1.00 | 20.41 | N |
| ATOM | 979 | CA | SER | A | 168 | 49.640 | 3.942 | 23.781 | 1.00 | 22.58 | C |
| ATOM | 980 | C | SER | A | 168 | 48.760 | 5.184 | 23.692 | 1.00 | 21.06 | C |
| ATOM | 981 | O | SER | A | 168 | 49.254 | 6.307 | 23.717 | 1.00 | 22.11 | O |
| ATOM | 982 | CB | SER | A | 168 | 50.764 | 4.005 | 22.745 | 1.00 | 25.82 | C |
| ATOM | 983 | OG | SER | A | 168 | 51.612 | 2.875 | 22.876 | 1.00 | 23.26 | O |
| ATOM | 984 | N | ASN | A | 169 | 47.454 | 4.966 | 23.590 | 1.00 | 20.29 | N |
| ATOM | 985 | CA | ASN | A | 169 | 46.479 | 6.049 | 23.519 | 1.00 | 23.72 | C |
| ATOM | 986 | C | ASN | A | 169 | 46.836 | 7.082 | 22.452 | 1.00 | 24.41 | C |
| ATOM | 987 | O | ASN | A | 169 | 46.775 | 8.292 | 22.693 | 1.00 | 21.39 | O |
| ATOM | 988 | CB | ASN | A | 169 | 46.363 | 6.708 | 24.901 | 1.00 | 25.88 | C |
| ATOM | 989 | CG | ASN | A | 169 | 45.176 | 7.642 | 25.010 | 1.00 | 27.35 | C |
| ATOM | 990 | OD1 | ASN | A | 169 | 44.116 | 7.395 | 24.432 | 1.00 | 32.18 | O |
| ATOM | 991 | ND2 | ASN | A | 169 | 45.342 | 8.715 | 25.775 | 1.00 | 27.13 | N |
| ATOM | 992 | N | LEU | A | 170 | 47.197 | 6.593 | 21.267 | 1.00 | 19.66 | N |
| ATOM | 993 | CA | LEU | A | 170 | 47.563 | 7.462 | 20.156 | 1.00 | 20.12 | C |
| ATOM | 994 | C | LEU | A | 170 | 46.361 | 7.971 | 19.368 | 1.00 | 21.48 | C |
| ATOM | 995 | O | LEU | A | 170 | 46.484 | 8.918 | 18.591 | 1.00 | 20.57 | O |
| ATOM | 996 | CB | LEU | A | 170 | 48.526 | 6.730 | 19.222 | 1.00 | 19.95 | C |
| ATOM | 997 | CG | LEU | A | 170 | 49.835 | 6.339 | 19.913 | 1.00 | 21.90 | C |
| ATOM | 998 | CD1 | LEU | A | 170 | 50.733 | 5.569 | 18.954 | 1.00 | 23.18 | C |
| ATOM | 999 | CD2 | LEU | A | 170 | 50.528 | 7.602 | 20.408 | 1.00 | 22.80 | C |
| ATOM | 1000 | N | VAL | A | 171 | 45.206 | 7.340 | 19.565 | 1.00 | 17.59 | N |
| ATOM | 1001 | CA | VAL | A | 171 | 43.976 | 7.745 | 18.882 | 1.00 | 18.70 | C |
| ATOM | 1002 | C | VAL | A | 171 | 42.963 | 8.139 | 19.951 | 1.00 | 20.85 | C |
| ATOM | 1003 | O | VAL | A | 171 | 42.214 | 7.301 | 20.459 | 1.00 | 21.66 | O |
| ATOM | 1004 | CB | VAL | A | 171 | 43.382 | 6.598 | 18.031 | 1.00 | 18.46 | C |
| ATOM | 1005 | CG1 | VAL | A | 171 | 42.058 | 7.037 | 17.416 | 1.00 | 15.74 | C |
| ATOM | 1006 | CG2 | VAL | A | 171 | 44.369 | 6.195 | 16.930 | 1.00 | 13.27 | C |
| ATOM | 1007 | N | PRO | A | 172 | 42.911 | 9.434 | 20.283 | 1.00 | 19.62 | N |
| ATOM | 1008 | CA | PRO | A | 172 | 42.020 | 10.017 | 21.294 | 1.00 | 22.13 | C |
| ATOM | 1009 | C | PRO | A | 172 | 40.554 | 9.597 | 21.237 | 1.00 | 20.16 | C |
| ATOM | 1010 | O | PRO | A | 172 | 39.947 | 9.323 | 22.275 | 1.00 | 20.65 | O |
| ATOM | 1011 | CB | PRO | A | 172 | 42.180 | 11.522 | 21.075 | 1.00 | 22.42 | C |
| ATOM | 1012 | CG | PRO | A | 172 | 43.549 | 11.647 | 20.518 | 1.00 | 25.62 | C |
| ATOM | 1013 | CD | PRO | A | 172 | 43.629 | 10.493 | 19.554 | 1.00 | 22.25 | C |
| ATOM | 1014 | N | SER | A | 173 | 39.989 | 9.542 | 20.034 | 1.00 | 16.26 | N |
| ATOM | 1015 | CA | SER | A | 173 | 38.574 | 9.192 | 19.873 | 1.00 | 17.32 | C |
| ATOM | 1016 | C | SER | A | 173 | 38.260 | 7.698 | 19.756 | 1.00 | 15.28 | C |
| ATOM | 1017 | O | SER | A | 173 | 37.147 | 7.319 | 19.380 | 1.00 | 12.95 | O |
| ATOM | 1018 | CB | SER | A | 173 | 38.004 | 9.920 | 18.653 | 1.00 | 16.36 | C |
| ATOM | 1019 | OG | SER | A | 173 | 38.663 | 9.505 | 17.466 | 1.00 | 16.37 | O |
| ATOM | 1020 | N | LEU | A | 174 | 39.224 | 6.849 | 20.090 | 1.00 | 16.85 | N |
| ATOM | 1021 | CA | LEU | A | 174 | 39.024 | 5.403 | 19.998 | 1.00 | 16.43 | C |
| ATOM | 1022 | C | LEU | A | 174 | 37.879 | 4.920 | 20.889 | 1.00 | 17.58 | C |
| ATOM | 1023 | O | LEU | A | 174 | 37.867 | 5.189 | 22.091 | 1.00 | 19.84 | O |
| ATOM | 1024 | CB | LEU | A | 174 | 40.309 | 4.680 | 20.398 | 1.00 | 14.64 | C |
| ATOM | 1025 | CG | LEU | A | 174 | 40.326 | 3.170 | 20.177 | 1.00 | 17.25 | C |
| ATOM | 1026 | CD1 | LEU | A | 174 | 40.278 | 2.870 | 18.683 | 1.00 | 17.09 | C |
| ATOM | 1027 | CD2 | LEU | A | 174 | 41.589 | 2.584 | 20.786 | 1.00 | 20.10 | C |
| ATOM | 1028 | N | LEU | A | 175 | 36.917 | 4.205 | 20.316 | 1.00 | 15.47 | N |
| ATOM | 1029 | CA | LEU | A | 175 | 35.813 | 3.695 | 21.120 | 1.00 | 17.56 | C |
| ATOM | 1030 | C | LEU | A | 175 | 35.743 | 2.167 | 21.168 | 1.00 | 17.45 | C |
| ATOM | 1031 | O | LEU | A | 175 | 34.948 | 1.605 | 21.914 | 1.00 | 15.99 | O |
| ATOM | 1032 | CB | LEU | A | 175 | 34.477 | 4.262 | 20.624 | 1.00 | 16.45 | C |
| ATOM | 1033 | CG | LEU | A | 175 | 34.087 | 4.175 | 19.145 | 1.00 | 20.84 | C |
| ATOM | 1034 | CD1 | LEU | A | 175 | 34.148 | 2.742 | 18.641 | 1.00 | 19.05 | C |
| ATOM | 1035 | CD2 | LEU | A | 175 | 32.677 | 4.731 | 18.984 | 1.00 | 21.90 | C |
| ATOM | 1036 | N | GLY | A | 176 | 36.576 | 1.495 | 20.378 | 1.00 | 17.50 | N |
| ATOM | 1037 | CA | GLY | A | 176 | 36.554 | 0.040 | 20.358 | 1.00 | 17.08 | C |
| ATOM | 1038 | C | GLY | A | 176 | 37.772 | −0.546 | 19.669 | 1.00 | 16.67 | C |
| ATOM | 1039 | O | GLY | A | 176 | 38.426 | 0.124 | 18.871 | 1.00 | 14.79 | O |
| ATOM | 1040 | N | LEU | A | 177 | 38.072 | −1.806 | 19.955 | 1.00 | 12.49 | N |
| ATOM | 1041 | CA | LEU | A | 177 | 39.251 | −2.438 | 19.376 | 1.00 | 15.26 | C |
| ATOM | 1042 | C | LEU | A | 177 | 38.984 | −3.884 | 18.988 | 1.00 | 16.88 | C |
| ATOM | 1043 | O | LEU | A | 177 | 38.437 | −4.639 | 19.780 | 1.00 | 15.39 | O |
| ATOM | 1044 | CB | LEU | A | 177 | 40.385 | −2.378 | 20.400 | 1.00 | 15.15 | C |
| ATOM | 1045 | CG | LEU | A | 177 | 41.671 | −3.168 | 20.163 | 1.00 | 18.03 | C |
| ATOM | 1046 | CD1 | LEU | A | 177 | 42.391 | −2.644 | 18.925 | 1.00 | 15.04 | C |
| ATOM | 1047 | CD2 | LEU | A | 177 | 42.559 | −3.041 | 21.400 | 1.00 | 14.78 | C |
| ATOM | 1048 | N | CYS | A | 178 | 39.372 | −4.268 | 17.773 | 1.00 | 14.83 | N |
| ATOM | 1049 | CA | CYS | A | 178 | 39.165 | −5.640 | 17.310 | 1.00 | 15.74 | C |
| ATOM | 1050 | C | CYS | A | 178 | 40.429 | −6.228 | 16.690 | 1.00 | 17.69 | C |
| ATOM | 1051 | O | CYS | A | 178 | 41.091 | −5.569 | 15.881 | 1.00 | 13.80 | O |
| ATOM | 1052 | CB | CYS | A | 178 | 38.040 | −5.698 | 16.261 | 1.00 | 14.15 | C |
| ATOM | 1053 | SG | CYS | A | 178 | 37.884 | −7.321 | 15.417 | 1.00 | 15.97 | S |
| ATOM | 1054 | N | MET | A | 179 | 40.757 | −7.460 | 17.082 | 1.00 | 15.81 | N |
| ATOM | 1055 | CA | MET | A | 179 | 41.912 | −8.181 | 16.541 | 1.00 | 15.78 | C |
| ATOM | 1056 | C | MET | A | 179 | 41.363 | −9.394 | 15.810 | 1.00 | 15.69 | C |
| ATOM | 1057 | O | MET | A | 179 | 40.507 | −10.096 | 16.345 | 1.00 | 12.43 | O |
| ATOM | 1058 | CB | MET | A | 179 | 42.818 | −8.719 | 17.645 | 1.00 | 19.20 | C |
| ATOM | 1059 | CG | MET | A | 179 | 43.454 | −7.702 | 18.540 | 1.00 | 23.80 | C |
| ATOM | 1060 | SD | MET | A | 179 | 44.485 | −8.604 | 19.698 | 1.00 | 24.27 | S |
| ATOM | 1061 | CE | MET | A | 179 | 45.956 | −8.646 | 18.812 | 1.00 | 23.07 | C |
| ATOM | 1062 | N | ILE | A | 180 | 41.861 | −9.660 | 14.609 | 1.00 | 15.03 | N |
| ATOM | 1063 | CA | ILE | A | 180 | 41.396 | −10.821 | 13.866 | 1.00 | 16.16 | C |
| ATOM | 1064 | C | ILE | A | 180 | 42.516 | −11.850 | 13.745 | 1.00 | 17.17 | C |
| ATOM | 1065 | O | ILE | A | 180 | 43.583 | −11.562 | 13.214 | 1.00 | 14.25 | O |
| ATOM | 1066 | CB | ILE | A | 180 | 40.905 | −10.430 | 12.456 | 1.00 | 15.35 | C |
| ATOM | 1067 | CG1 | ILE | A | 180 | 39.770 | −9.407 | 12.583 | 1.00 | 13.75 | C |
| ATOM | 1068 | CG2 | ILE | A | 180 | 40.429 | −11.675 | 11.710 | 1.00 | 16.03 | C |
| ATOM | 1069 | CD1 | ILE | A | 180 | 39.193 | −8.916 | 11.259 | 1.00 | 15.26 | C |
| ATOM | 1070 | N | ASP | A | 181 | 42.265 | −13.047 | 14.263 | 1.00 | 16.63 | N |
| ATOM | 1071 | CA | ASP | A | 181 | 43.241 | −14.132 | 14.208 | 1.00 | 20.76 | C |
| ATOM | 1072 | C | ASP | A | 181 | 44.617 | −13.794 | 14.753 | 1.00 | 21.80 | C |
| ATOM | 1073 | O | ASP | A | 181 | 45.625 | −14.169 | 14.165 | 1.00 | 27.74 | O |
| ATOM | 1074 | CB | ASP | A | 181 | 43.388 | −14.645 | 12.773 | 1.00 | 22.23 | C |
| ATOM | 1075 | CG | ASP | A | 181 | 42.514 | −15.848 | 12.499 | 1.00 | 24.36 | C |
| ATOM | 1076 | OD1 | ASP | A | 181 | 42.043 | −16.465 | 13.486 | 1.00 | 22.02 | O |
| ATOM | 1077 | OD2 | ASP | A | 181 | 42.315 | −16.187 | 11.310 | 1.00 | 22.17 | O |
| ATOM | 1078 | N | VAL | A | 182 | 44.661 | −13.080 | 15.871 | 1.00 | 21.36 | N |
| ATOM | 1079 | CA | VAL | A | 182 | 45.931 | −12.725 | 16.494 | 1.00 | 24.38 | C |
| ATOM | 1080 | C | VAL | A | 182 | 45.940 | −13.218 | 17.927 | 1.00 | 20.35 | C |
| ATOM | 1081 | O | VAL | A | 182 | 45.194 | −12.709 | 18.766 | 1.00 | 25.82 | O |
| ATOM | 1082 | CB | VAL | A | 182 | 46.172 | −11.195 | 16.556 | 1.00 | 25.60 | C |
| ATOM | 1083 | CG1 | VAL | A | 182 | 47.479 | −10.920 | 17.296 | 1.00 | 30.82 | C |
| ATOM | 1084 | CG2 | VAL | A | 182 | 46.234 | −10.609 | 15.177 | 1.00 | 22.44 | C |
| ATOM | 1085 | N | VAL | A | 183 | 46.773 | −14.214 | 18.209 | 1.00 | 17.87 | N |
| ATOM | 1086 | CA | VAL | A | 183 | 46.881 | −14.734 | 19.565 | 1.00 | 16.85 | C |
| ATOM | 1087 | C | VAL | A | 183 | 48.364 | −14.841 | 19.886 | 1.00 | 15.62 | C |
| ATOM | 1088 | O | VAL | A | 183 | 49.067 | −15.676 | 19.319 | 1.00 | 13.79 | O |
| ATOM | 1089 | CB | VAL | A | 183 | 46.189 | −16.119 | 19.706 | 1.00 | 16.62 | C |
| ATOM | 1090 | CG1 | VAL | A | 183 | 46.481 | −16.718 | 21.080 | 1.00 | 18.17 | C |
| ATOM | 1091 | CG2 | VAL | A | 183 | 44.676 | −15.960 | 19.531 | 1.00 | 15.93 | C |
| ATOM | 1092 | N | GLU | A | 184 | 48.823 | −13.980 | 20.794 | 1.00 | 13.60 | N |
| ATOM | 1093 | CA | GLU | A | 184 | 50.226 | −13.928 | 21.195 | 1.00 | 16.20 | C |
| ATOM | 1094 | C | GLU | A | 184 | 50.882 | −15.285 | 21.353 | 1.00 | 15.45 | C |
| ATOM | 1095 | O | GLU | A | 184 | 51.944 | −15.530 | 20.780 | 1.00 | 16.15 | O |
| ATOM | 1096 | CB | GLU | A | 184 | 50.400 | −13.158 | 22.514 | 1.00 | 13.63 | C |
| ATOM | 1097 | CG | GLU | A | 184 | 51.827 | −13.251 | 23.056 | 1.00 | 16.39 | C |
| ATOM | 1098 | CD | GLU | A | 184 | 52.035 | −12.500 | 24.358 | 1.00 | 19.46 | C |
| ATOM | 1099 | OE1 | GLU | A | 184 | 51.058 | −11.928 | 24.882 | 1.00 | 19.20 | O |
| ATOM | 1100 | OE2 | GLU | A | 184 | 53.184 | −12.488 | 24.856 | 1.00 | 17.55 | O |
| ATOM | 1101 | N | GLY | A | 185 | 50.252 | −16.151 | 22.143 | 1.00 | 14.82 | N |
| ATOM | 1102 | CA | GLY | A | 185 | 50.798 | −17.473 | 22.400 | 1.00 | 17.66 | C |
| ATOM | 1103 | C | GLY | A | 185 | 51.294 | −18.244 | 21.190 | 1.00 | 19.85 | C |
| ATOM | 1104 | O | GLY | A | 185 | 52.325 | −18.912 | 21.264 | 1.00 | 21.38 | O |
| ATOM | 1105 | N | THR | A | 186 | 50.571 | −18.150 | 20.078 | 1.00 | 18.40 | N |
| ATOM | 1106 | CA | THR | A | 186 | 50.934 | −18.876 | 18.862 | 1.00 | 18.70 | C |
| ATOM | 1107 | C | THR | A | 186 | 51.304 | −17.976 | 17.679 | 1.00 | 18.95 | C |
| ATOM | 1108 | O | THR | A | 186 | 51.573 | −18.470 | 16.582 | 1.00 | 19.04 | O |
| ATOM | 1109 | CB | THR | A | 186 | 49.775 | −19.790 | 18.413 | 1.00 | 17.57 | C |
| ATOM | 1110 | OG1 | THR | A | 186 | 48.570 | −19.016 | 18.327 | 1.00 | 19.50 | O |
| ATOM | 1111 | CG2 | THR | A | 186 | 49.569 | −20.938 | 19.404 | 1.00 | 20.04 | C |
| ATOM | 1112 | N | ALA | A | 187 | 51.336 | −16.668 | 17.903 | 1.00 | 14.83 | N |
| ATOM | 1113 | CA | ALA | A | 187 | 51.640 | −15.721 | 16.834 | 1.00 | 15.69 | C |
| ATOM | 1114 | C | ALA | A | 187 | 52.921 | −16.024 | 16.061 | 1.00 | 16.49 | C |
| ATOM | 1115 | O | ALA | A | 187 | 52.904 | −16.068 | 14.827 | 1.00 | 17.76 | O |
| ATOM | 1116 | CB | ALA | A | 187 | 51.691 | −14.302 | 17.396 | 1.00 | 15.11 | C |
| ATOM | 1117 | N | MET | A | 188 | 54.029 | −16.231 | 16.768 | 1.00 | 15.84 | N |
| ATOM | 1118 | CA | MET | A | 188 | 55.295 | −16.525 | 16.097 | 1.00 | 18.16 | C |
| ATOM | 1119 | C | MET | A | 188 | 55.205 | −17.791 | 15.253 | 1.00 | 17.55 | C |
| ATOM | 1120 | O | MET | A | 188 | 55.716 | −17.837 | 14.135 | 1.00 | 17.70 | O |
| ATOM | 1121 | CB | MET | A | 188 | 56.428 | −16.676 | 17.116 | 1.00 | 17.89 | C |
| ATOM | 1122 | CG | MET | A | 188 | 56.820 | −15.388 | 17.809 | 1.00 | 27.24 | C |
| ATOM | 1123 | SD | MET | A | 188 | 57.403 | −14.157 | 16.638 | 1.00 | 32.68 | S |
| ATOM | 1124 | CE | MET | A | 188 | 58.979 | −14.843 | 16.168 | 1.00 | 33.64 | C |
| ATOM | 1125 | N | ASP | A | 189 | 54.567 | −18.823 | 15.792 | 1.00 | 17.44 | N |
| ATOM | 1126 | CA | ASP | A | 189 | 54.429 | −20.074 | 15.061 | 1.00 | 16.98 | C |
| ATOM | 1127 | C | ASP | A | 189 | 53.553 | −19.896 | 13.831 | 1.00 | 20.15 | C |
| ATOM | 1128 | O | ASP | A | 189 | 53.795 | −20.508 | 12.790 | 1.00 | 14.82 | O |
| ATOM | 1129 | CB | ASP | A | 189 | 53.855 | −21.163 | 15.969 | 1.00 | 18.19 | C |
| ATOM | 1130 | CG | ASP | A | 189 | 54.836 | −21.593 | 17.043 | 1.00 | 20.16 | C |
| ATOM | 1131 | OD1 | ASP | A | 189 | 56.059 | −21.514 | 16.791 | 1.00 | 20.81 | O |
| ATOM | 1132 | OD2 | ASP | A | 189 | 54.389 | −22.020 | 18.127 | 1.00 | 20.99 | O |
| ATOM | 1133 | N | ALA | A | 190 | 52.533 | −19.054 | 13.945 | 1.00 | 18.32 | N |
| ATOM | 1134 | CA | ALA | A | 190 | 51.659 | −18.797 | 12.806 | 1.00 | 17.80 | C |
| ATOM | 1135 | C | ALA | A | 190 | 52.476 | −18.143 | 11.683 | 1.00 | 18.03 | C |
| ATOM | 1136 | O | ALA | A | 190 | 52.369 | −18.534 | 10.522 | 1.00 | 20.23 | O |
| ATOM | 1137 | CB | ALA | A | 190 | 50.495 | −17.886 | 13.227 | 1.00 | 15.90 | C |
| ATOM | 1138 | N | LEU | A | 191 | 53.299 | −17.156 | 12.027 | 1.00 | 16.49 | N |
| ATOM | 1139 | CA | LEU | A | 191 | 54.109 | −16.480 | 11.016 | 1.00 | 16.44 | C |
| ATOM | 1140 | C | LEU | A | 191 | 55.191 | −17.374 | 10.415 | 1.00 | 20.86 | C |
| ATOM | 1141 | O | LEU | A | 191 | 55.486 | −17.293 | 9.221 | 1.00 | 19.66 | O |
| ATOM | 1142 | CB | LEU | A | 191 | 54.748 | −15.222 | 11.594 | 1.00 | 19.57 | C |
| ATOM | 1143 | CG | LEU | A | 191 | 53.818 | −14.004 | 11.666 | 1.00 | 23.21 | C |
| ATOM | 1144 | CD1 | LEU | A | 191 | 54.602 | −12.788 | 12.126 | 1.00 | 27.24 | C |
| ATOM | 1145 | CD2 | LEU | A | 191 | 53.226 | −13.737 | 10.300 | 1.00 | 26.58 | C |
| ATOM | 1146 | N | ASN | A | 192 | 55.793 | −18.221 | 11.236 | 1.00 | 19.99 | N |
| ATOM | 1147 | CA | ASN | A | 192 | 56.825 | −19.115 | 10.732 | 1.00 | 23.44 | C |
| ATOM | 1148 | C | ASN | A | 192 | 56.249 | −20.157 | 9.777 | 1.00 | 21.64 | C |
| ATOM | 1149 | O | ASN | A | 192 | 56.877 | −20.503 | 8.776 | 1.00 | 18.67 | O |
| ATOM | 1150 | CB | ASN | A | 192 | 57.543 | −19.784 | 11.899 | 1.00 | 24.76 | C |
| ATOM | 1151 | CG | ASN | A | 192 | 58.543 | −18.861 | 12.552 | 1.00 | 30.64 | C |
| ATOM | 1152 | OD1 | ASN | A | 192 | 58.751 | −18.900 | 13.764 | 1.00 | 36.54 | O |
| ATOM | 1153 | ND2 | ASN | A | 192 | 59.181 | −18.025 | 11.742 | 1.00 | 29.76 | N |
| ATOM | 1154 | N | SER | A | 193 | 55.049 | −20.648 | 10.068 | 1.00 | 21.78 | N |
| ATOM | 1155 | CA | SER | A | 193 | 54.438 | −21.641 | 9.192 | 1.00 | 26.02 | C |
| ATOM | 1156 | C | SER | A | 193 | 54.207 | −21.032 | 7.810 | 1.00 | 27.62 | C |
| ATOM | 1157 | O | SER | A | 193 | 54.207 | −21.741 | 6.807 | 1.00 | 30.36 | O |
| ATOM | 1158 | CB | SER | A | 193 | 53.103 | −22.131 | 9.762 | 1.00 | 27.30 | C |
| ATOM | 1159 | OG | SER | A | 193 | 52.116 | −21.119 | 9.702 | 1.00 | 32.12 | O |
| ATOM | 1160 | N | MET | A | 194 | 54.023 | −19.716 | 7.768 | 1.00 | 30.59 | N |
| ATOM | 1161 | CA | MET | A | 194 | 53.781 | −18.996 | 6.516 | 1.00 | 35.42 | C |
| ATOM | 1162 | C | MET | A | 194 | 55.022 | −18.814 | 5.649 | 1.00 | 34.89 | C |
| ATOM | 1163 | O | MET | A | 194 | 54.945 | −18.892 | 4.423 | 1.00 | 34.91 | O |
| ATOM | 1164 | CB | MET | A | 194 | 53.185 | −17.616 | 6.814 | 1.00 | 37.77 | C |
| ATOM | 1165 | CG | MET | A | 194 | 51.665 | −17.545 | 6.790 | 1.00 | 46.51 | C |
| ATOM | 1166 | SD | MET | A | 194 | 50.983 | −17.509 | 5.106 | 1.00 | 49.88 | S |
| ATOM | 1167 | CE | MET | A | 194 | 51.286 | −15.797 | 4.668 | 1.00 | 52.59 | C |
| ATOM | 1168 | N | GLN | A | 195 | 56.163 | −18.571 | 6.284 | 1.00 | 36.93 | N |
| ATOM | 1169 | CA | GLN | A | 195 | 57.409 | −18.341 | 5.554 | 1.00 | 35.62 | C |
| ATOM | 1170 | C | GLN | A | 195 | 57.748 | −19.379 | 4.495 | 1.00 | 33.07 | C |
| ATOM | 1171 | O | GLN | A | 195 | 58.267 | −19.040 | 3.436 | 1.00 | 27.58 | O |
| ATOM | 1172 | CB | GLN | A | 195 | 58.572 | −18.201 | 6.537 | 1.00 | 39.35 | C |
| ATOM | 1173 | CG | GLN | A | 195 | 58.459 | −16.963 | 7.404 | 1.00 | 45.87 | C |
| ATOM | 1174 | CD | GLN | A | 195 | 58.421 | −15.683 | 6.584 | 1.00 | 50.75 | C |
| ATOM | 1175 | OE1 | GLN | A | 195 | 58.043 | −14.620 | 7.083 | 1.00 | 52.42 | O |
| ATOM | 1176 | NE2 | GLN | A | 195 | 58.821 | −15.778 | 5.319 | 1.00 | 51.40 | N |
| ATOM | 1177 | N | ASN | A | 196 | 57.460 | −20.642 | 4.774 | 1.00 | 33.80 | N |
| ATOM | 1178 | CA | ASN | A | 196 | 57.746 | −21.705 | 3.821 | 1.00 | 33.15 | C |
| ATOM | 1179 | C | ASN | A | 196 | 56.961 | −21.439 | 2.531 | 1.00 | 29.59 | C |
| ATOM | 1180 | O | ASN | A | 196 | 57.501 | −21.517 | 1.434 | 1.00 | 32.98 | O |
| ATOM | 1181 | CB | ASN | A | 196 | 57.355 | −23.051 | 4.433 | 1.00 | 41.24 | C |
| ATOM | 1182 | CG | ASN | A | 196 | 57.808 | −23.183 | 5.885 | 1.00 | 45.64 | C |
| ATOM | 1183 | OD1 | ASN | A | 196 | 59.004 | −23.140 | 6.181 | 1.00 | 51.23 | O |
| ATOM | 1184 | ND2 | ASN | A | 196 | 56.853 | −23.335 | 6.793 | 1.00 | 44.46 | N |
| ATOM | 1185 | N | PHE | A | 197 | 55.686 | −21.106 | 2.677 | 1.00 | 28.16 | N |
| ATOM | 1186 | CA | PHE | A | 197 | 54.833 | −20.797 | 1.534 | 1.00 | 24.02 | C |
| ATOM | 1187 | C | PHE | A | 197 | 55.347 | −19.545 | 0.836 | 1.00 | 23.54 | C |
| ATOM | 1188 | O | PHE | A | 197 | 55.508 | −19.512 | −0.386 | 1.00 | 19.96 | O |
| ATOM | 1189 | CB | PHE | A | 197 | 53.400 | −20.542 | 2.003 | 1.00 | 22.54 | C |
| ATOM | 1190 | CG | PHE | A | 197 | 52.536 | −19.849 | 0.976 | 1.00 | 26.22 | C |
| ATOM | 1191 | CD1 | PHE | A | 197 | 52.111 | −20.523 | −0.166 | 1.00 | 25.70 | C |
| ATOM | 1192 | CD2 | PHE | A | 197 | 52.152 | −18.520 | 1.152 | 1.00 | 25.09 | C |
| ATOM | 1193 | CE1 | PHE | A | 197 | 51.312 | −19.885 | −1.120 | 1.00 | 28.43 | C |
| ATOM | 1194 | CE2 | PHE | A | 197 | 51.352 | −17.872 | 0.203 | 1.00 | 24.28 | C |
| ATOM | 1195 | CZ | PHE | A | 197 | 50.931 | −18.558 | −0.934 | 1.00 | 22.54 | C |
| ATOM | 1196 | N | LEU | A | 198 | 55.608 | −18.508 | 1.624 | 1.00 | 24.69 | N |
| ATOM | 1197 | CA | LEU | A | 198 | 56.080 | −17.249 | 1.065 | 1.00 | 26.35 | C |
| ATOM | 1198 | C | LEU | A | 198 | 57.344 | −17.417 | 0.227 | 1.00 | 27.36 | C |
| ATOM | 1199 | O | LEU | A | 198 | 57.433 | −16.890 | −0.885 | 1.00 | 28.29 | O |
| ATOM | 1200 | CB | LEU | A | 198 | 56.305 | −16.228 | 2.184 | 1.00 | 24.11 | C |
| ATOM | 1201 | CG | LEU | A | 198 | 55.047 | −15.889 | 2.995 | 1.00 | 25.48 | C |
| ATOM | 1202 | CD1 | LEU | A | 198 | 55.396 | −14.913 | 4.118 | 1.00 | 26.12 | C |
| ATOM | 1203 | CD2 | LEU | A | 198 | 53.982 | −15.292 | 2.077 | 1.00 | 27.31 | C |
| ATOM | 1204 | N | ARG | A | 199 | 58.316 | −18.160 | 0.738 | 1.00 | 25.61 | N |
| ATOM | 1205 | CA | ARG | A | 199 | 59.555 | −18.350 | −0.005 | 1.00 | 28.56 | C |
| ATOM | 1206 | C | ARG | A | 199 | 59.364 | −19.126 | −1.310 | 1.00 | 28.28 | C |
| ATOM | 1207 | O | ARG | A | 199 | 60.100 | −18.915 | −2.274 | 1.00 | 29.35 | O |
| ATOM | 1208 | CB | ARG | A | 199 | 60.586 | −19.058 | 0.869 | 1.00 | 35.49 | C |
| ATOM | 1209 | CG | ARG | A | 199 | 60.126 | −20.398 | 1.403 | 1.00 | 44.92 | C |
| ATOM | 1210 | CD | ARG | A | 199 | 61.090 | −20.920 | 2.457 | 1.00 | 52.08 | C |
| ATOM | 1211 | NE | ARG | A | 199 | 61.359 | −19.907 | 3.475 | 1.00 | 55.31 | N |
| ATOM | 1212 | CZ | ARG | A | 199 | 61.991 | −20.143 | 4.619 | 1.00 | 56.54 | C |
| ATOM | 1213 | NE1 | ARG | A | 199 | 62.421 | −21.367 | 4.898 | 1.00 | 58.45 | N |
| ATOM | 1214 | NH2 | ARG | A | 199 | 62.201 | −19.154 | 5.480 | 1.00 | 54.10 | N |
| ATOM | 1215 | N | GLY | A | 200 | 58.370 | −20.007 | −1.347 | 1.00 | 26.20 | N |
| ATOM | 1216 | CA | GLY | A | 200 | 58.135 | −20.796 | −2.546 | 1.00 | 23.08 | C |
| ATOM | 1217 | C | GLY | A | 200 | 57.294 | −20.152 | −3.637 | 1.00 | 23.22 | C |
| ATOM | 1218 | O | GLY | A | 200 | 57.165 | −20.712 | −4.727 | 1.00 | 20.45 | O |
| ATOM | 1219 | N | ARG | A | 201 | 56.721 | −18.983 | −3.366 | 1.00 | 20.79 | N |
| ATOM | 1220 | CA | ARG | A | 201 | 55.882 | −18.304 | −4.354 | 1.00 | 19.19 | C |
| ATOM | 1221 | C | ARG | A | 201 | 56.670 | −17.841 | −5.573 | 1.00 | 18.75 | C |
| ATOM | 1222 | O | ARG | A | 201 | 57.898 | −17.743 | −5.534 | 1.00 | 19.66 | O |
| ATOM | 1223 | CB | ARG | A | 201 | 55.210 | −17.067 | −3.734 | 1.00 | 19.79 | C |
| ATOM | 1224 | CG | ARG | A | 201 | 54.295 | −17.339 | −2.553 | 1.00 | 20.14 | C |
| ATOM | 1225 | CD | ARG | A | 201 | 53.595 | −16.056 | −2.092 | 1.00 | 21.35 | C |
| ATOM | 1226 | NE | ARG | A | 201 | 54.550 | −15.006 | −1.725 | 1.00 | 21.79 | N |
| ATOM | 1227 | CZ | ARG | A | 201 | 54.200 | −13.812 | −1.252 | 1.00 | 24.82 | C |
| ATOM | 1228 | NH1 | ARG | A | 201 | 52.918 | −13.508 | −1.082 | 1.00 | 24.12 | N |
| ATOM | 1229 | NH2 | ARG | A | 201 | 55.128 | −12.922 | −0.951 | 1.00 | 25.35 | N |
| ATOM | 1230 | N | PRO | A | 202 | 55.971 | −17.561 | −6.684 | 1.00 | 18.13 | N |
| ATOM | 1231 | CA | PRO | A | 202 | 56.679 | −17.093 | −7.878 | 1.00 | 22.02 | C |
| ATOM | 1232 | C | PRO | A | 202 | 57.381 | −15.789 | −7.484 | 1.00 | 25.18 | C |
| ATOM | 1233 | O | PRO | A | 202 | 56.906 | −15.077 | −6.594 | 1.00 | 24.64 | O |
| ATOM | 1234 | CB | PRO | A | 202 | 55.550 | −16.854 | −8.870 | 1.00 | 23.33 | C |
| ATOM | 1235 | CG | PRO | A | 202 | 54.559 | −17.919 | −8.501 | 1.00 | 25.37 | C |
| ATOM | 1236 | CD | PRO | A | 202 | 54.560 | −17.858 | −6.991 | 1.00 | 21.24 | C |
| ATOM | 1237 | N | LYS | A | 203 | 58.505 | −15.477 | −8.118 | 1.00 | 23.45 | N |
| ATOM | 1238 | CA | LYS | A | 203 | 59.221 | −14.249 | −7.785 | 1.00 | 27.01 | C |
| ATOM | 1239 | C | LYS | A | 203 | 58.715 | −13.052 | −8.582 | 1.00 | 26.77 | C |
| ATOM | 1240 | O | LYS | A | 203 | 58.913 | −11.911 | −8.175 | 1.00 | 30.72 | O |
| ATOM | 1241 | CB | LYS | A | 203 | 60.728 | −14.425 | −8.002 | 1.00 | 28.81 | C |
| ATOM | 1242 | CG | LYS | A | 203 | 61.333 | −15.481 | −7.092 | 1.00 | 35.13 | C |
| ATOM | 1243 | CD | LYS | A | 203 | 62.852 | −15.455 | −7.095 | 1.00 | 38.60 | C |
| ATOM | 1244 | CE | LYS | A | 203 | 63.400 | −16.559 | −6.201 | 1.00 | 40.89 | C |
| ATOM | 1245 | NZ | LYS | A | 203 | 62.785 | −16.522 | −4.835 | 1.00 | 41.02 | N |
| ATOM | 1246 | N | THR | A | 204 | 58.066 | −13.315 | −9.714 | 1.00 | 24.72 | N |
| ATOM | 1247 | CA | THR | A | 204 | 57.522 | −12.254 | −10.560 | 1.00 | 25.00 | C |
| ATOM | 1248 | C | THR | A | 204 | 56.312 | −12.778 | −11.314 | 1.00 | 25.61 | C |
| ATOM | 1249 | O | THR | A | 204 | 56.102 | −13.984 | −11.403 | 1.00 | 28.60 | O |
| ATOM | 1250 | CB | THR | A | 204 | 58.539 | −11.768 | −11.623 | 1.00 | 26.69 | C |
| ATOM | 1251 | OG1 | THR | A | 204 | 58.763 | −12.811 | −12.579 | 1.00 | 26.83 | O |
| ATOM | 1252 | CG2 | THR | A | 204 | 59.862 | −11.389 | −10.978 | 1.00 | 25.37 | C |
| ATOM | 1253 | N | PHE | A | 205 | 55.520 | −11.863 | −11.857 | 1.00 | 24.51 | N |
| ATOM | 1254 | CA | PHE | A | 205 | 54.343 | −12.219 | −12.636 | 1.00 | 25.30 | C |
| ATOM | 1255 | C | PHE | A | 205 | 54.370 | −11.390 | −13.909 | 1.00 | 26.59 | C |
| ATOM | 1256 | O | PHE | A | 205 | 54.766 | −10.228 | −13.888 | 1.00 | 27.61 | O |
| ATOM | 1257 | CB | PHE | A | 205 | 53.064 | −11.924 | −11.850 | 1.00 | 23.47 | C |
| ATOM | 1258 | CG | PHE | A | 205 | 52.874 | −12.814 | −10.651 | 1.00 | 21.98 | C |
| ATOM | 1259 | CD1 | PHE | A | 205 | 52.439 | −14.130 | −10.808 | 1.00 | 22.18 | C |
| ATOM | 1260 | CD2 | PHE | A | 205 | 53.161 | −12.349 | −9.373 | 1.00 | 20.88 | C |
| ATOM | 1261 | CE1 | PHE | A | 205 | 52.295 | −14.971 | −9.704 | 1.00 | 20.77 | C |
| ATOM | 1262 | CE2 | PHE | A | 205 | 53.019 | −13.183 | −8.258 | 1.00 | 18.86 | C |
| ATOM | 1263 | CZ | PHE | A | 205 | 52.585 | −14.496 | −8.428 | 1.00 | 20.26 | C |
| ATOM | 1264 | N | LYS | A | 206 | 53.948 | −11.991 | −15.014 | 1.00 | 27.54 | N |
| ATOM | 1265 | CA | LYS | A | 206 | 53.937 | −11.302 | −16.293 | 1.00 | 33.32 | C |
| ATOM | 1266 | C | LYS | A | 206 | 52.981 | −10.131 | −16.275 | 1.00 | 31.89 | C |
| ATOM | 1267 | O | LYS | A | 206 | 53.277 | −9.074 | −16.826 | 1.00 | 35.12 | O |
| ATOM | 1268 | CB | LYS | A | 206 | 53.542 | −12.271 | −17.403 | 1.00 | 37.69 | C |
| ATOM | 1269 | CG | LYS | A | 206 | 54.461 | −13.470 | −17.488 | 1.00 | 45.20 | C |
| ATOM | 1270 | CD | LYS | A | 206 | 53.945 | −14.504 | −18.467 | 1.00 | 49.95 | C |
| ATOM | 1271 | CE | LYS | A | 206 | 54.837 | −15.731 | −18.449 | 1.00 | 54.82 | C |
| ATOM | 1272 | NZ | LYS | A | 206 | 54.981 | −16.272 | −17.064 | 1.00 | 57.12 | N |
| ATOM | 1273 | N | SER | A | 207 | 51.834 | −10.320 | −15.633 | 1.00 | 29.48 | N |
| ATOM | 1274 | CA | SER | A | 207 | 50.832 | −9.268 | −15.553 | 1.00 | 26.41 | C |
| ATOM | 1275 | C | SER | A | 207 | 50.114 | −9.316 | −14.218 | 1.00 | 28.00 | C |
| ATOM | 1276 | O | SER | A | 207 | 50.290 | −10.248 | −13.436 | 1.00 | 23.33 | O |
| ATOM | 1277 | CB | SER | A | 207 | 49.796 | −9.437 | −16.661 | 1.00 | 27.57 | C |
| ATOM | 1278 | OG | SER | A | 207 | 49.043 | −10.621 | −16.459 | 1.00 | 23.25 | O |
| ATOM | 1279 | N | LEU | A | 208 | 49.288 | −8.307 | −13.972 | 1.00 | 25.72 | N |
| ATOM | 1280 | CA | LEU | A | 208 | 48.519 | −8.239 | −12.742 | 1.00 | 26.71 | C |
| ATOM | 1281 | C | LEU | A | 208 | 47.465 | −9.341 | −12.783 | 1.00 | 25.60 | C |
| ATOM | 1282 | O | LEU | A | 208 | 47.159 | −9.963 | −11.767 | 1.00 | 21.75 | O |
| ATOM | 1283 | CB | LEU | A | 208 | 47.858 | −6.868 | −12.624 | 1.00 | 29.05 | C |
| ATOM | 1284 | CG | LEU | A | 208 | 47.131 | −6.514 | −11.331 | 1.00 | 32.02 | C |
| ATOM | 1285 | CD1 | LEU | A | 208 | 47.982 | −6.880 | −10.118 | 1.00 | 33.86 | C |
| ATOM | 1286 | CD2 | LEU | A | 208 | 46.831 | −5.022 | −11.342 | 1.00 | 32.11 | C |
| ATOM | 1287 | N | GLU | A | 209 | 46.922 | −9.593 | −13.969 | 1.00 | 19.86 | N |
| ATOM | 1288 | CA | GLU | A | 209 | 45.917 | −10.633 | −14.122 | 1.00 | 20.88 | C |
| ATOM | 1289 | C | GLU | A | 209 | 46.481 | −12.003 | −13.771 | 1.00 | 19.27 | C |
| ATOM | 1290 | O | GLU | A | 209 | 45.782 | −12.837 | −13.180 | 1.00 | 19.24 | O |
| ATOM | 1291 | CB | GLU | A | 209 | 45.384 | −10.669 | −15.553 | 1.00 | 23.64 | C |
| ATOM | 1292 | CG | GLU | A | 209 | 44.239 | −11.645 | −15.721 | 1.00 | 31.06 | C |
| ATOM | 1293 | CD | GLU | A | 209 | 43.612 | −11.583 | −17.099 | 1.00 | 37.11 | C |
| ATOM | 1294 | OE1 | GLU | A | 209 | 43.336 | −10.464 | −17.577 | 1.00 | 40.01 | O |
| ATOM | 1295 | OE2 | GLU | A | 209 | 43.383 | −12.654 | −17.697 | 1.00 | 41.34 | O |
| ATOM | 1296 | N | ASN | A | 210 | 47.737 | −12.250 | −14.133 | 1.00 | 17.89 | N |
| ATOM | 1297 | CA | ASN | A | 210 | 48.327 | −13.549 | −13.831 | 1.00 | 20.24 | C |
| ATOM | 1298 | C | ASN | A | 210 | 48.597 | −13.687 | −12.345 | 1.00 | 18.45 | C |
| ATOM | 1299 | O | ASN | A | 210 | 48.492 | −14.783 | −11.799 | 1.00 | 18.42 | O |
| ATOM | 1300 | CB | ASN | A | 210 | 49.627 | −13.784 | −14.601 | 1.00 | 25.33 | C |
| ATOM | 1301 | CG | ASN | A | 210 | 50.169 | −15.198 | −14.393 | 1.00 | 34.23 | C |
| ATOM | 1302 | OD1 | ASN | A | 210 | 49.500 | −16.186 | −14.714 | 1.00 | 32.18 | O |
| ATOM | 1303 | ND2 | ASN | A | 210 | 51.374 | −15.298 | −13.843 | 1.00 | 33.31 | N |
| ATOM | 1304 | N | ALA | A | 211 | 48.941 | −12.580 | −11.687 | 1.00 | 19.97 | N |
| ATOM | 1305 | CA | ALA | A | 211 | 49.200 | −12.616 | −10.245 | 1.00 | 19.59 | C |
| ATOM | 1306 | C | ALA | A | 211 | 47.896 | −12.975 | −9.545 | 1.00 | 18.35 | C |
| ATOM | 1307 | O | ALA | A | 211 | 47.871 | −13.782 | −8.609 | 1.00 | 17.43 | O |
| ATOM | 1308 | CB | ALA | A | 211 | 49.713 | −11.251 | −9.752 | 1.00 | 15.58 | C |
| ATOM | 1309 | N | ILE | A | 212 | 46.806 | −12.369 | −10.007 | 1.00 | 18.11 | N |
| ATOM | 1310 | CA | ILE | A | 212 | 45.496 | −12.635 | −9.434 | 1.00 | 16.80 | C |
| ATOM | 1311 | C | ILE | A | 212 | 45.110 | −14.089 | −9.671 | 1.00 | 17.28 | C |
| ATOM | 1312 | O | ILE | A | 212 | 44.637 | −14.775 | −8.758 | 1.00 | 15.80 | O |
| ATOM | 1313 | CB | ILE | A | 212 | 44.414 | −11.711 | −10.051 | 1.00 | 18.07 | C |
| ATOM | 1314 | CG1 | ILE | A | 212 | 44.650 | −10.266 | −9.599 | 1.00 | 17.58 | C |
| ATOM | 1315 | CG2 | ILE | A | 212 | 43.016 | −12.188 | −9.646 | 1.00 | 17.28 | C |
| ATOM | 1316 | CD1 | ILE | A | 212 | 43.900 | −9.222 | −10.416 | 1.00 | 19.46 | C |
| ATOM | 1317 | N | GLU | A | 213 | 45.317 | −14.570 | −10.891 | 1.00 | 17.02 | N |
| ATOM | 1318 | CA | GLU | A | 213 | 44.960 | −15.946 | −11.191 | 1.00 | 19.02 | C |
| ATOM | 1319 | C | GLU | A | 213 | 45.784 | −16.893 | −10.325 | 1.00 | 19.15 | C |
| ATOM | 1320 | O | GLU | A | 213 | 45.268 | −17.884 | −9.809 | 1.00 | 18.97 | O |
| ATOM | 1321 | CB | GLU | A | 213 | 45.179 | −16.252 | −12.674 | 1.00 | 19.13 | C |
| ATOM | 1322 | CG | GLU | A | 213 | 44.655 | −17.622 | −13.099 | 1.00 | 17.86 | C |
| ATOM | 1323 | CD | GLU | A | 213 | 44.530 | −17.747 | −14.606 | 1.00 | 23.74 | C |
| ATOM | 1324 | OE1 | GLU | A | 213 | 44.871 | −16.776 | −15.310 | 1.00 | 25.42 | O |
| ATOM | 1325 | OE2 | GLU | A | 213 | 44.088 | −18.811 | −15.085 | 1.00 | 24.12 | O |
| ATOM | 1326 | N | TRP | A | 214 | 47.064 | −16.585 | −10.155 | 1.00 | 20.08 | N |
| ATOM | 1327 | CA | TRP | A | 214 | 47.921 | −17.434 | −9.334 | 1.00 | 19.85 | C |
| ATOM | 1328 | C | TRP | A | 214 | 47.464 | −17.411 | −7.871 | 1.00 | 20.83 | C |
| ATOM | 1329 | O | TRP | A | 214 | 47.401 | −18.457 | −7.215 | 1.00 | 15.58 | O |
| ATOM | 1330 | CB | TRP | A | 214 | 49.379 | −16.969 | −9.411 | 1.00 | 19.54 | C |
| ATOM | 1331 | CG | TRP | A | 214 | 50.278 | −17.757 | −8.496 | 1.00 | 21.03 | C |
| ATOM | 1332 | CD1 | TRP | A | 214 | 50.927 | −18.923 | −8.787 | 1.00 | 23.98 | C |
| ATOM | 1333 | CD2 | TRP | A | 214 | 50.515 | −17.505 | −7.105 | 1.00 | 21.14 | C |
| ATOM | 1334 | NE1 | TRP | A | 214 | 51.544 | −19.417 | −7.664 | 1.00 | 22.88 | N |
| ATOM | 1335 | CE2 | TRP | A | 214 | 51.306 | −18.567 | −6.617 | 1.00 | 22.59 | C |
| ATOM | 1336 | CE3 | TRP | A | 214 | 50.129 | −16.487 | −6.223 | 1.00 | 19.22 | C |
| ATOM | 1337 | CZ2 | TRP | A | 214 | 51.719 | −18.643 | −5.283 | 1.00 | 22.34 | C |
| ATOM | 1338 | CZ3 | TRP | A | 214 | 50.540 | −16.564 | −4.895 | 1.00 | 22.70 | C |
| ATOM | 1339 | CH2 | TRP | A | 214 | 51.327 | −17.635 | −4.441 | 1.00 | 20.80 | C |
| ATOM | 1340 | N | SER | A | 215 | 47.143 | −16.221 | −7.361 | 1.00 | 17.81 | N |
| ATOM | 1341 | CA | SER | A | 215 | 46.730 | −16.086 | −5.965 | 1.00 | 17.79 | C |
| ATOM | 1342 | C | SER | A | 215 | 45.506 | −16.935 | −5.639 | 1.00 | 17.96 | C |
| ATOM | 1343 | O | SER | A | 215 | 45.348 | −17.397 | −4.503 | 1.00 | 16.46 | O |
| ATOM | 1344 | CB | SER | A | 215 | 46.449 | −14.620 | −5.615 | 1.00 | 19.02 | C |
| ATOM | 1345 | OG | SER | A | 215 | 45.211 | −14.184 | −6.151 | 1.00 | 18.45 | O |
| ATOM | 1346 | N | VAL | A | 216 | 44.642 | −17.137 | −6.628 | 1.00 | 13.74 | N |
| ATOM | 1347 | CA | VAL | A | 216 | 43.445 | −17.944 | −6.427 | 1.00 | 15.24 | C |
| ATOM | 1348 | C | VAL | A | 216 | 43.735 | −19.427 | −6.616 | 1.00 | 16.13 | C |
| ATOM | 1349 | O | VAL | A | 216 | 43.406 | −20.249 | −5.757 | 1.00 | 16.30 | O |
| ATOM | 1350 | CB | VAL | A | 216 | 42.310 | −17.549 | −7.409 | 1.00 | 16.18 | C |
| ATOM | 1351 | CG1 | VAL | A | 216 | 41.141 | −18.509 | −7.265 | 1.00 | 17.25 | C |
| ATOM | 1352 | CG2 | VAL | A | 216 | 41.837 | −16.132 | −7.119 | 1.00 | 17.33 | C |
| ATOM | 1353 | N | LYS | A | 217 | 44.368 | −19.772 | −7.733 | 1.00 | 14.91 | N |
| ATOM | 1354 | CA | LYS | A | 217 | 44.637 | −21.179 | −8.006 | 1.00 | 16.31 | C |
| ATOM | 1355 | C | LYS | A | 217 | 45.639 | −21.822 | −7.050 | 1.00 | 16.46 | C |
| ATOM | 1356 | O | LYS | A | 217 | 45.618 | −23.035 | −6.844 | 1.00 | 18.54 | O |
| ATOM | 1357 | CB | LYS | A | 217 | 45.054 | −21.344 | −9.463 | 1.00 | 16.74 | C |
| ATOM | 1358 | CG | LYS | A | 217 | 43.872 | −21.129 | −10.408 | 1.00 | 20.53 | C |
| ATOM | 1359 | CD | LYS | A | 217 | 44.251 | −21.207 | −11.879 | 1.00 | 21.64 | C |
| ATOM | 1360 | CE | LYS | A | 217 | 43.007 | −21.088 | −12.762 | 1.00 | 22.17 | C |
| ATOM | 1361 | NZ | LYS | A | 217 | 43.326 | −21.086 | −14.225 | 1.00 | 22.62 | N |
| ATOM | 1362 | N | SER | A | 218 | 46.488 | −21.004 | −6.439 | 1.00 | 17.20 | N |
| ATOM | 1363 | CA | SER | A | 218 | 47.477 | −21.498 | −5.483 | 1.00 | 17.38 | C |
| ATOM | 1364 | C | SER | A | 218 | 46.792 | −21.778 | −4.154 | 1.00 | 19.97 | C |
| ATOM | 1365 | O | SER | A | 218 | 47.316 | −22.514 | −3.319 | 1.00 | 18.84 | O |
| ATOM | 1366 | CB | SER | A | 218 | 48.555 | −20.447 | −5.248 | 1.00 | 18.17 | C |
| ATOM | 1367 | OG | SER | A | 218 | 48.007 | −19.334 | −4.566 | 1.00 | 18.66 | O |
| ATOM | 1368 | N | GLY | A | 219 | 45.625 | −21.171 | −3.961 | 1.00 | 18.80 | N |
| ATOM | 1369 | CA | GLY | A | 219 | 44.896 | −21.347 | −2.719 | 1.00 | 18.78 | C |
| ATOM | 1370 | C | GLY | A | 219 | 45.164 | −20.221 | −1.734 | 1.00 | 21.18 | C |
| ATOM | 1371 | O | GLY | A | 219 | 44.667 | −20.243 | −0.607 | 1.00 | 20.35 | O |
| ATOM | 1372 | N | GLN | A | 220 | 45.941 | −19.221 | −2.140 | 1.00 | 17.94 | N |
| ATOM | 1373 | CA | GLN | A | 220 | 46.241 | −18.121 | −1.225 | 1.00 | 19.00 | C |
| ATOM | 1374 | C | GLN | A | 220 | 44.994 | −17.293 | −0.919 | 1.00 | 21.56 | C |
| ATOM | 1375 | O | GLN | A | 220 | 44.729 | −16.962 | 0.238 | 1.00 | 20.12 | O |
| ATOM | 1376 | CB | GLN | A | 220 | 47.331 | −17.211 | −1.799 | 1.00 | 20.29 | C |
| ATOM | 1377 | CG | GLN | A | 220 | 47.782 | −16.143 | −0.816 | 1.00 | 27.01 | C |
| ATOM | 1378 | CD | GLN | A | 220 | 48.696 | −15.105 | −1.438 | 1.00 | 32.50 | C |
| ATOM | 1379 | OE1 | GLN | A | 220 | 49.804 | −15.418 | −1.880 | 1.00 | 34.54 | O |
| ATOM | 1380 | NE2 | GLN | A | 220 | 48.235 | −13.857 | −1.472 | 1.00 | 30.52 | N |
| ATOM | 1381 | N | ILE | A | 221 | 44.236 | −16.955 | −1.958 | 1.00 | 19.10 | N |
| ATOM | 1382 | CA | ILE | A | 221 | 43.010 | −16.177 | −1.796 | 1.00 | 20.55 | C |
| ATOM | 1383 | C | ILE | A | 221 | 41.883 | −16.976 | −2.452 | 1.00 | 23.76 | C |
| ATOM | 1384 | O | ILE | A | 221 | 41.783 | −17.030 | −3.677 | 1.00 | 21.11 | O |
| ATOM | 1385 | CB | ILE | A | 221 | 43.127 | −14.787 | −2.472 | 1.00 | 22.38 | C |
| ATOM | 1386 | CG1 | ILE | A | 221 | 44.366 | −14.054 | −1.951 | 1.00 | 18.19 | C |
| ATOM | 1387 | CG2 | ILE | A | 221 | 41.880 | −13.945 | −2.167 | 1.00 | 19.30 | C |
| ATOM | 1388 | CD1 | ILE | A | 221 | 44.670 | −12.769 | −2.690 | 1.00 | 21.60 | C |
| ATOM | 1389 | N | ARG | A | 222 | 41.052 | −17.605 | −1.628 | 1.00 | 22.93 | N |
| ATOM | 1390 | CA | ARG | A | 222 | 39.948 | −18.425 | −2.114 | 1.00 | 28.46 | C |
| ATOM | 1391 | C | ARG | A | 222 | 38.860 | −17.634 | −2.822 | 1.00 | 27.87 | C |
| ATOM | 1392 | O | ARG | A | 222 | 38.289 | −18.085 | −3.819 | 1.00 | 30.68 | O |
| ATOM | 1393 | CB | ARG | A | 222 | 39.300 | −19.175 | −0.953 | 1.00 | 32.06 | C |
| ATOM | 1394 | CG | ARG | A | 222 | 39.757 | −20.595 | −0.772 | 1.00 | 41.00 | C |
| ATOM | 1395 | CD | ARG | A | 222 | 41.142 | −20.683 | −0.180 | 1.00 | 43.56 | C |
| ATOM | 1396 | NE | ARG | A | 222 | 41.438 | −22.070 | 0.164 | 1.00 | 52.35 | N |
| ATOM | 1397 | CZ | ARG | A | 222 | 42.516 | −22.471 | 0.825 | 1.00 | 51.78 | C |
| ATOM | 1398 | NH1 | ARG | A | 222 | 43.422 | −21.589 | 1.220 | 1.00 | 53.87 | N |
| ATOM | 1399 | NH2 | ARG | A | 222 | 42.678 | −23.759 | 1.103 | 1.00 | 52.96 | N |
| ATOM | 1400 | N | ASN | A | 223 | 38.573 | −16.459 | −2.286 | 1.00 | 23.29 | N |
| ATOM | 1401 | CA | ASN | A | 223 | 37.535 | −15.599 | −2.814 | 1.00 | 19.16 | C |
| ATOM | 1402 | C | ASN | A | 223 | 38.008 | −14.810 | −4.032 | 1.00 | 17.48 | C |
| ATOM | 1403 | O | ASN | A | 223 | 38.734 | −13.824 | −3.913 | 1.00 | 14.74 | O |
| ATOM | 1404 | CB | ASN | A | 223 | 37.063 | −14.668 | −1.690 | 1.00 | 21.65 | C |
| ATOM | 1405 | CG | ASN | A | 223 | 35.905 | −13.787 | −2.101 | 1.00 | 25.87 | C |
| ATOM | 1406 | OD1 | ASN | A | 223 | 35.256 | −14.023 | −3.118 | 1.00 | 29.42 | O |
| ATOM | 1407 | ND2 | ASN | A | 223 | 35.632 | −12.766 | −1.299 | 1.00 | 27.88 | N |
| ATOM | 1408 | N | LEU | A | 224 | 37.594 | −15.261 | −5.212 | 1.00 | 17.00 | N |
| ATOM | 1409 | CA | LEU | A | 224 | 37.975 | −14.600 | −6.455 | 1.00 | 17.76 | C |
| ATOM | 1410 | C | LEU | A | 224 | 37.677 | −13.104 | −6.431 | 1.00 | 16.47 | C |
| ATOM | 1411 | O | LEU | A | 224 | 38.502 | −12.295 | −6.857 | 1.00 | 19.10 | O |
| ATOM | 1412 | CB | LEU | A | 224 | 37.247 | −15.246 | −7.639 | 1.00 | 18.32 | C |
| ATOM | 1413 | CG | LEU | A | 224 | 37.343 | −14.544 | −9.000 | 1.00 | 16.11 | C |
| ATOM | 1414 | CD1 | LEU | A | 224 | 38.792 | −14.419 | −9.442 | 1.00 | 19.05 | C |
| ATOM | 1415 | CD2 | LEU | A | 224 | 36.537 | −15.350 | −10.023 | 1.00 | 18.28 | C |
| ATOM | 1416 | N | GLU | A | 225 | 36.503 | −12.735 | −5.928 | 1.00 | 17.12 | N |
| ATOM | 1417 | CA | GLU | A | 225 | 36.126 | −11.324 | −5.888 | 1.00 | 18.64 | C |
| ATOM | 1418 | C | GLU | A | 225 | 37.162 | −10.495 | −5.147 | 1.00 | 17.51 | C |
| ATOM | 1419 | O | GLU | A | 225 | 37.618 | −9.459 | −5.640 | 1.00 | 15.19 | O |
| ATOM | 1420 | CB | GLU | A | 225 | 34.752 | −11.136 | −5.228 | 1.00 | 18.68 | C |
| ATOM | 1421 | CG | GLU | A | 225 | 34.242 | −9.691 | −5.349 | 1.00 | 21.39 | C |
| ATOM | 1422 | CD | GLU | A | 225 | 32.949 | −9.427 | −4.596 | 1.00 | 21.27 | C |
| ATOM | 1423 | OE1 | GLU | A | 225 | 32.350 | −10.382 | −4.060 | 1.00 | 20.97 | O |
| ATOM | 1424 | OE2 | GLU | A | 225 | 32.531 | −8.250 | −4.546 | 1.00 | 22.28 | O |
| ATOM | 1425 | N | SER | A | 226 | 37.546 | −10.958 | −3.963 | 1.00 | 16.16 | N |
| ATOM | 1426 | CA | SER | A | 226 | 38.530 | −10.237 | −3.171 | 1.00 | 18.33 | C |
| ATOM | 1427 | C | SER | A | 226 | 39.888 | −10.211 | −3.869 | 1.00 | 16.37 | C |
| ATOM | 1428 | O | SER | A | 226 | 40.552 | −9.180 | −3.904 | 1.00 | 20.10 | O |
| ATOM | 1429 | CB | SER | A | 226 | 38.660 | −10.875 | −1.786 | 1.00 | 18.62 | C |
| ATOM | 1430 | OG | SER | A | 226 | 39.433 | −10.054 | −0.932 | 1.00 | 26.62 | O |
| ATOM | 1431 | N | ALA | A | 227 | 40.303 | −11.341 | −4.434 | 1.00 | 16.98 | N |
| ATOM | 1432 | CA | ALA | A | 227 | 41.596 | −11.400 | −5.118 | 1.00 | 16.67 | C |
| ATOM | 1433 | C | ALA | A | 227 | 41.742 | −10.382 | −6.249 | 1.00 | 16.21 | C |
| ATOM | 1434 | O | ALA | A | 227 | 42.794 | −9.760 | −6.392 | 1.00 | 15.56 | O |
| ATOM | 1435 | CB | ALA | A | 227 | 41.837 | −12.809 | −5.666 | 1.00 | 16.04 | C |
| ATOM | 1436 | N | ARG | A | 228 | 40.696 | −10.206 | −7.055 | 1.00 | 15.90 | N |
| ATOM | 1437 | CA | ARG | A | 228 | 40.773 | −9.277 | −8.184 | 1.00 | 15.82 | C |
| ATOM | 1438 | C | ARG | A | 228 | 41.088 | −7.846 | −7.773 | 1.00 | 18.90 | C |
| ATOM | 1439 | O | ARG | A | 228 | 41.725 | −7.100 | −8.526 | 1.00 | 18.60 | O |
| ATOM | 1440 | CB | ARG | A | 228 | 39.470 | −9.298 | −8.991 | 1.00 | 14.59 | C |
| ATOM | 1441 | CG | ARG | A | 228 | 39.140 | −10.644 | −9.615 | 1.00 | 17.61 | C |
| ATOM | 1442 | CD | ARG | A | 228 | 37.823 | −10.580 | −10.377 | 1.00 | 17.75 | C |
| ATOM | 1443 | NE | ARG | A | 228 | 37.933 | −9.796 | −11.604 | 1.00 | 19.73 | N |
| ATOM | 1444 | CZ | ARG | A | 228 | 36.899 | −9.263 | −12.253 | 1.00 | 24.60 | C |
| ATOM | 1445 | NH1 | ARG | A | 228 | 35.668 | −9.424 | −11.789 | 1.00 | 23.83 | N |
| ATOM | 1446 | NH2 | ARG | A | 228 | 37.094 | −8.573 | −13.370 | 1.00 | 22.48 | N |
| ATOM | 1447 | N | VAL | A | 229 | 40.649 | −7.461 | −6.579 | 1.00 | 16.27 | N |
| ATOM | 1448 | CA | VAL | A | 229 | 40.902 | −6.110 | −6.103 | 1.00 | 16.16 | C |
| ATOM | 1449 | C | VAL | A | 229 | 42.079 | −6.012 | −5.132 | 1.00 | 19.33 | C |
| ATOM | 1450 | O | VAL | A | 229 | 42.849 | −5.053 | −5.190 | 1.00 | 17.37 | O |
| ATOM | 1451 | CB | VAL | A | 229 | 39.622 | −5.506 | −5.439 | 1.00 | 18.46 | C |
| ATOM | 1452 | CG1 | VAL | A | 229 | 39.134 | −6.405 | −4.310 | 1.00 | 15.29 | C |
| ATOM | 1453 | CG2 | VAL | A | 229 | 39.911 | −4.095 | −4.914 | 1.00 | 19.06 | C |
| ATOM | 1454 | N | SER | A | 230 | 42.252 | −7.005 | −4.262 | 1.00 | 17.65 | N |
| ATOM | 1455 | CA | SER | A | 230 | 43.336 | −6.927 | −3.281 | 1.00 | 17.49 | C |
| ATOM | 1456 | C | SER | A | 230 | 44.742 | −7.212 | −3.794 | 1.00 | 18.87 | C |
| ATOM | 1457 | O | SER | A | 230 | 45.701 | −6.625 | −3.299 | 1.00 | 16.90 | O |
| ATOM | 1458 | CB | SER | A | 230 | 43.066 | −7.850 | −2.089 | 1.00 | 17.68 | C |
| ATOM | 1459 | OG | SER | A | 230 | 43.267 | −9.203 | −2.437 | 1.00 | 24.08 | O |
| ATOM | 1460 | N | MET | A | 231 | 44.881 | −8.100 | −4.773 | 1.00 | 16.59 | N |
| ATOM | 1461 | CA | MET | A | 231 | 46.220 | −8.423 | −5.264 | 1.00 | 22.01 | C |
| ATOM | 1462 | C | MET | A | 231 | 47.028 | −7.240 | −5.777 | 1.00 | 21.30 | C |
| ATOM | 1463 | O | MET | A | 231 | 48.250 | −7.237 | −5.669 | 1.00 | 21.33 | O |
| ATOM | 1464 | CB | MET | A | 231 | 46.166 | −9.512 | −6.337 | 1.00 | 23.22 | C |
| ATOM | 1465 | CG | MET | A | 231 | 46.230 | −10.921 | −5.765 | 1.00 | 27.57 | C |
| ATOM | 1466 | SD | MET | A | 231 | 47.737 | −11.241 | −4.770 | 1.00 | 23.22 | S |
| ATOM | 1467 | CE | MET | A | 231 | 48.943 | −11.238 | −6.038 | 1.00 | 23.66 | C |
| ATOM | 1468 | N | VAL | A | 232 | 46.364 | −6.227 | −6.317 | 1.00 | 23.41 | N |
| ATOM | 1469 | CA | VAL | A | 232 | 47.111 | −5.083 | −6.821 | 1.00 | 27.60 | C |
| ATOM | 1470 | C | VAL | A | 232 | 47.871 | −4.427 | −5.658 | 1.00 | 25.01 | C |
| ATOM | 1471 | O | VAL | A | 232 | 48.903 | −3.798 | −5.862 | 1.00 | 28.86 | O |
| ATOM | 1472 | CB | VAL | A | 232 | 46.175 | −4.048 | −7.530 | 1.00 | 29.36 | C |
| ATOM | 1473 | CG1 | VAL | A | 232 | 44.996 | −4.770 | −8.183 | 1.00 | 35.08 | C |
| ATOM | 1474 | CG2 | VAL | A | 232 | 45.700 | −3.003 | −6.565 | 1.00 | 31.05 | C |
| ATOM | 1475 | N | GLY | A | 233 | 47.370 | −4.598 | −4.436 | 1.00 | 25.39 | N |
| ATOM | 1476 | CA | GLY | A | 233 | 48.038 | −4.020 | −3.277 | 1.00 | 17.34 | C |
| ATOM | 1477 | C | GLY | A | 233 | 49.222 | −4.842 | −2.784 | 1.00 | 19.59 | C |
| ATOM | 1478 | O | GLY | A | 233 | 50.088 | −4.336 | −2.068 | 1.00 | 20.02 | O |
| ATOM | 1479 | N | GLN | A | 234 | 49.272 | −6.116 | −3.165 | 1.00 | 18.78 | N |
| ATOM | 1480 | CA | GLN | A | 234 | 50.361 | −6.983 | −2.730 | 1.00 | 18.45 | C |
| ATOM | 1481 | C | GLN | A | 234 | 51.564 | −6.954 | −3.661 | 1.00 | 19.54 | C |
| ATOM | 1482 | O | GLN | A | 234 | 52.655 | −7.364 | −3.271 | 1.00 | 16.32 | O |
| ATOM | 1483 | CB | GLN | A | 234 | 49.871 | −8.427 | −2.586 | 1.00 | 23.72 | C |
| ATOM | 1484 | CG | GLN | A | 234 | 48.853 | −8.616 | −1.475 | 1.00 | 24.97 | C |
| ATOM | 1485 | CD | GLN | A | 234 | 48.396 | −10.051 | −1.339 | 1.00 | 28.25 | C |
| ATOM | 1486 | OE1 | GLN | A | 234 | 49.210 | −10.968 | −1.256 | 1.00 | 28.36 | O |
| ATOM | 1487 | NE2 | GLN | A | 234 | 47.081 | −10.253 | −1.308 | 1.00 | 34.75 | N |
| ATOM | 1488 | N | VAL | A | 235 | 51.370 | −6.471 | −4.885 | 1.00 | 21.64 | N |
| ATOM | 1489 | CA | VAL | A | 235 | 52.463 | −6.415 | −5.849 | 1.00 | 23.67 | C |
| ATOM | 1490 | C | VAL | A | 235 | 52.787 | −5.005 | −6.331 | 1.00 | 29.50 | C |
| ATOM | 1491 | O | VAL | A | 235 | 52.078 | −4.044 | −6.022 | 1.00 | 30.25 | O |
| ATOM | 1492 | CB | VAL | A | 235 | 52.163 | −7.292 | −7.080 | 1.00 | 22.83 | C |
| ATOM | 1493 | CG1 | VAL | A | 235 | 52.069 | −8.751 | −6.663 | 1.00 | 20.90 | C |
| ATOM | 1494 | CG2 | VAL | A | 235 | 50.860 | −6.849 | −7.733 | 1.00 | 25.12 | C |
| ATOM | 1495 | N | LYS | A | 236 | 53.877 | −4.901 | −7.082 | 1.00 | 27.66 | N |
| ATOM | 1496 | CA | LYS | A | 236 | 54.337 | −3.637 | −7.643 | 1.00 | 29.97 | C |
| ATOM | 1497 | C | LYS | A | 236 | 55.117 | −3.974 | −8.905 | 1.00 | 32.61 | C |
| ATOM | 1498 | O | LYS | A | 236 | 55.608 | −5.095 | −9.059 | 1.00 | 27.44 | O |
| ATOM | 1499 | CB | LYS | A | 236 | 55.268 | −2.922 | −6.666 | 1.00 | 28.70 | C |
| ATOM | 1500 | CG | LYS | A | 236 | 56.556 | −3.681 | −6.404 | 1.00 | 33.02 | C |
| ATOM | 1501 | CD | LYS | A | 236 | 57.491 | −2.909 | −5.494 | 1.00 | 36.34 | C |
| ATOM | 1502 | CE | LYS | A | 236 | 58.665 | −3.771 | −5.070 | 1.00 | 40.55 | C |
| ATOM | 1503 | NZ | LYS | A | 236 | 59.391 | −4.310 | −6.250 | 1.00 | 44.48 | N |
| ATOM | 1504 | N | GLN | A | 237 | 55.237 | −3.010 | −9.808 | 1.00 | 35.12 | N |
| ATOM | 1505 | CA | GLN | A | 237 | 55.982 | −3.244 | −11.035 | 1.00 | 40.38 | C |
| ATOM | 1506 | C | GLN | A | 237 | 57.469 | −3.355 | −10.727 | 1.00 | 42.26 | C |
| ATOM | 1507 | O | GLN | A | 237 | 58.011 | −2.560 | −9.962 | 1.00 | 41.72 | O |
| ATOM | 1508 | CB | GLN | A | 237 | 55.734 | −2.113 | −12.024 | 1.00 | 41.59 | C |
| ATOM | 1509 | CG | GLN | A | 237 | 54.290 | −2.008 | −12.446 | 1.00 | 46.95 | C |
| ATOM | 1510 | CD | GLN | A | 237 | 54.105 | −1.073 | −13.614 | 1.00 | 51.77 | C |
| ATOM | 1511 | OE1 | GLN | A | 237 | 54.418 | 0.117 | −13.528 | 1.00 | 53.87 | O |
| ATOM | 1512 | NE2 | GLN | A | 237 | 53.598 | −1.606 | −14.723 | 1.00 | 50.39 | N |
| ATOM | 1513 | N | CYS | A | 238 | 58.118 | −4.350 | −11.322 | 1.00 | 45.06 | N |
| ATOM | 1514 | CA | CYS | A | 238 | 59.543 | −4.581 | −11.114 | 1.00 | 50.31 | C |
| ATOM | 1515 | C | CYS | A | 238 | 60.391 | −3.347 | −11.401 | 1.00 | 55.20 | C |
| ATOM | 1516 | O | CYS | A | 238 | 60.204 | −2.674 | −12.416 | 1.00 | 54.14 | O |
| ATOM | 1517 | CB | CYS | A | 238 | 60.026 | −5.731 | −11.998 | 1.00 | 49.81 | C |
| ATOM | 1518 | SG | CYS | A | 238 | 59.284 | −7.324 | −11.624 | 1.00 | 46.31 | S |
| ATOM | 1519 | N | GLU | A | 239 | 61.326 | −3.066 | −10.495 | 1.00 | 59.74 | N |
| ATOM | 1520 | CA | GLU | A | 239 | 62.228 | −1.927 | −10.628 | 1.00 | 63.53 | C |
| ATOM | 1521 | C | GLU | A | 239 | 61.469 | −0.606 | −10.589 | 1.00 | 64.73 | C |
| ATOM | 1522 | O | GLU | A | 239 | 61.270 | −0.026 | −9.520 | 1.00 | 66.94 | O |
| ATOM | 1523 | CB | GLU | A | 239 | 63.012 | −2.037 | −11.936 | 1.00 | 65.26 | C |
| ATOM | 1524 | CG | GLU | A | 239 | 63.757 | −3.352 | −12.085 | 1.00 | 68.22 | C |
| ATOM | 1525 | CD | GLU | A | 239 | 64.385 | −3.517 | −13.453 | 1.00 | 71.01 | C |
| ATOM | 1526 | OE1 | GLU | A | 239 | 65.042 | −4.555 | −13.679 | 1.00 | 72.89 | O |
| ATOM | 1527 | OE2 | GLU | A | 239 | 64.220 | −2.612 | −14.302 | 1.00 | 72.11 | O |
| ATOM | 1528 | N | PRO | A | 284 | 56.254 | −6.241 | −15.788 | 1.00 | 36.86 | N |
| ATOM | 1529 | CA | PRO | A | 284 | 56.103 | −7.405 | −14.907 | 1.00 | 35.63 | C |
| ATOM | 1530 | C | PRO | A | 284 | 56.015 | −6.975 | −13.446 | 1.00 | 32.22 | C |
| ATOM | 1531 | O | PRO | A | 284 | 56.575 | −5.943 | −13.059 | 1.00 | 30.38 | O |
| ATOM | 1532 | CB | PRO | A | 284 | 57.363 | −8.211 | −15.195 | 1.00 | 35.88 | C |
| ATOM | 1533 | CG | PRO | A | 284 | 58.382 | −7.129 | −15.375 | 1.00 | 37.97 | C |
| ATOM | 1534 | CD | PRO | A | 284 | 57.650 | −6.114 | −16.245 | 1.00 | 38.20 | C |
| ATOM | 1535 | N | TYR | A | 285 | 55.323 | −7.768 | −12.637 | 1.00 | 28.55 | N |
| ATOM | 1536 | CA | TYR | A | 285 | 55.164 | −7.445 | −11.228 | 1.00 | 26.70 | C |
| ATOM | 1537 | C | TYR | A | 285 | 55.925 | −8.357 | −10.286 | 1.00 | 27.36 | C |
| ATOM | 1538 | O | TYR | A | 285 | 56.246 | −9.502 | −10.613 | 1.00 | 25.87 | O |
| ATOM | 1539 | CB | TYR | A | 285 | 53.688 | −7.485 | −10.832 | 1.00 | 29.86 | C |
| ATOM | 1540 | CG | TYR | A | 285 | 52.834 | −6.405 | −11.448 | 1.00 | 31.99 | C |
| ATOM | 1541 | CD1 | TYR | A | 285 | 52.493 | −6.444 | −12.801 | 1.00 | 35.00 | C |
| ATOM | 1542 | CD2 | TYR | A | 285 | 52.358 | −5.343 | −10.675 | 1.00 | 32.72 | C |
| ATOM | 1543 | CE1 | TYR | A | 285 | 51.697 | −5.452 | −13.373 | 1.00 | 36.58 | C |
| ATOM | 1544 | CE2 | TYR | A | 285 | 51.560 | −4.344 | −11.236 | 1.00 | 36.29 | C |
| ATOM | 1545 | CZ | TYR | A | 285 | 51.234 | −4.406 | −12.585 | 1.00 | 38.11 | C |
| ATOM | 1546 | OH | TYR | A | 285 | 50.453 | −3.424 | −13.148 | 1.00 | 41.99 | O |
| ATOM | 1547 | N | THR | A | 286 | 56.202 | −7.831 | −9.101 | 1.00 | 21.82 | N |
| ATOM | 1548 | CA | THR | A | 286 | 56.878 | −8.588 | −8.069 | 1.00 | 23.68 | C |
| ATOM | 1549 | C | THR | A | 286 | 56.232 | −8.192 | −6.745 | 1.00 | 24.47 | C |
| ATOM | 1550 | O | THR | A | 286 | 55.433 | −7.249 | −6.696 | 1.00 | 20.75 | O |
| ATOM | 1551 | CB | THR | A | 286 | 58.379 | −8.287 | −8.033 | 1.00 | 22.89 | C |
| ATOM | 1552 | OG1 | THR | A | 286 | 59.020 | −9.204 | −7.137 | 1.00 | 24.10 | O |
| ATOM | 1553 | CG2 | THR | A | 286 | 58.624 | −6.866 | −7.572 | 1.00 | 23.37 | C |
| ATOM | 1554 | N | TRP | A | 287 | 56.566 | −8.907 | −5.677 | 1.00 | 22.96 | N |
| ATOM | 1555 | CA | TRP | A | 287 | 55.983 | −8.620 | −4.374 | 1.00 | 24.08 | C |
| ATOM | 1556 | C | TRP | A | 287 | 56.382 | −7.265 | −3.813 | 1.00 | 21.86 | C |
| ATOM | 1557 | O | TRP | A | 287 | 57.543 | −6.871 | −3.862 | 1.00 | 18.89 | O |
| ATOM | 1558 | CB | TRP | A | 287 | 56.338 | −9.734 | −3.387 | 1.00 | 25.13 | C |
| ATOM | 1559 | CG | TRP | A | 287 | 55.859 | −11.066 | −3.875 | 1.00 | 23.94 | C |
| ATOM | 1560 | CD1 | TRP | A | 287 | 56.614 | −12.046 | −4.454 | 1.00 | 24.56 | C |
| ATOM | 1561 | CD2 | TRP | A | 287 | 54.500 | −11.518 | −3.934 | 1.00 | 24.51 | C |
| ATOM | 1562 | NE1 | TRP | A | 287 | 55.807 | −13.077 | −4.876 | 1.00 | 24.88 | N |
| ATOM | 1563 | CE2 | TRP | A | 287 | 54.506 | −12.779 | −4.569 | 1.00 | 23.99 | C |
| ATOM | 1564 | CE3 | TRP | A | 287 | 53.276 | −10.978 | −3.514 | 1.00 | 22.28 | C |
| ATOM | 1565 | CZ2 | TRP | A | 287 | 53.336 | −13.510 | −4.796 | 1.00 | 23.34 | C |
| ATOM | 1566 | CZ3 | TRP | A | 287 | 52.113 | −11.704 | −3.737 | 1.00 | 23.54 | C |
| ATOM | 1567 | CH2 | TRP | A | 287 | 52.153 | −12.958 | −4.374 | 1.00 | 24.68 | C |
| ATOM | 1568 | N | ARG | A | 288 | 55.398 | −6.544 | −3.287 | 1.00 | 23.70 | N |
| ATOM | 1569 | CA | ARG | A | 288 | 55.646 | −5.229 | −2.713 | 1.00 | 23.30 | C |
| ATOM | 1570 | C | ARG | A | 288 | 56.682 | −5.285 | −1.584 | 1.00 | 23.27 | C |
| ATOM | 1571 | O | ARG | A | 288 | 57.504 | −4.378 | −1.435 | 1.00 | 22.72 | O |
| ATOM | 1572 | CB | ARG | A | 288 | 54.332 | −4.637 | −2.186 | 1.00 | 24.71 | C |
| ATOM | 1573 | CG | ARG | A | 288 | 54.505 | −3.355 | −1.388 | 1.00 | 27.38 | C |
| ATOM | 1574 | CD | ARG | A | 288 | 55.016 | −2.217 | −2.263 | 1.00 | 35.57 | C |
| ATOM | 1575 | NE | ARG | A | 288 | 54.042 | −1.819 | −3.278 | 1.00 | 36.13 | N |
| ATOM | 1576 | CZ | ARG | A | 288 | 54.252 | −0.860 | −4.177 | 1.00 | 38.13 | C |
| ATOM | 1577 | NH1 | ARG | A | 288 | 55.406 | −0.201 | −4.189 | 1.00 | 35.22 | N |
| ATOM | 1578 | NH2 | ARG | A | 288 | 53.306 | −0.550 | −5.056 | 1.00 | 33.86 | N |
| ATOM | 1579 | N | ILE | A | 289 | 56.649 | −6.354 | −0.796 | 1.00 | 24.74 | N |
| ATOM | 1580 | CA | ILE | A | 289 | 57.575 | −6.492 | 0.318 | 1.00 | 24.98 | C |
| ATOM | 1581 | C | ILE | A | 289 | 57.955 | −7.945 | 0.606 | 1.00 | 27.14 | C |
| ATOM | 1582 | O | ILE | A | 289 | 57.165 | −8.861 | 0.372 | 1.00 | 26.14 | O |
| ATOM | 1583 | CB | ILE | A | 289 | 56.956 | −5.875 | 1.602 | 1.00 | 25.77 | C |
| ATOM | 1584 | CG1 | ILE | A | 289 | 57.989 | −5.848 | 2.730 | 1.00 | 28.36 | C |
| ATOM | 1585 | CG2 | ILE | A | 289 | 55.730 | −6.684 | 2.036 | 1.00 | 23.70 | C |
| ATOM | 1586 | CD1 | ILE | A | 289 | 57.493 | −5.163 | 3.994 | 1.00 | 27.52 | C |
| ATOM | 1587 | N | GLU | A | 290 | 59.179 | −8.153 | 1.088 | 1.00 | 29.57 | N |
| ATOM | 1588 | CA | GLU | A | 290 | 59.638 | −9.492 | 1.464 | 1.00 | 33.15 | C |
| ATOM | 1589 | C | GLU | A | 290 | 59.438 | −9.511 | 2.970 | 1.00 | 32.32 | C |
| ATOM | 1590 | O | GLU | A | 290 | 60.236 | −8.943 | 3.710 | 1.00 | 29.29 | O |
| ATOM | 1591 | CB | GLU | A | 290 | 61.127 | −9.693 | 1.154 | 1.00 | 40.56 | C |
| ATOM | 1592 | CG | GLU | A | 290 | 61.479 | −9.690 | −0.322 | 1.00 | 49.96 | C |
| ATOM | 1593 | CD | GLU | A | 290 | 61.424 | −8.304 | −0.933 | 1.00 | 57.50 | C |
| ATOM | 1594 | OE1 | GLU | A | 290 | 62.231 | −7.443 | −0.517 | 1.00 | 61.79 | O |
| ATOM | 1595 | OE2 | GLU | A | 290 | 60.578 | −8.073 | −1.828 | 1.00 | 61.43 | O |
| ATOM | 1596 | N | LEU | A | 291 | 58.366 | −10.153 | 3.419 | 1.00 | 32.19 | N |
| ATOM | 1597 | CA | LEU | A | 291 | 58.051 | −10.197 | 4.839 | 1.00 | 34.43 | C |
| ATOM | 1598 | C | LEU | A | 291 | 59.167 | −10.783 | 5.710 | 1.00 | 35.08 | C |
| ATOM | 1599 | O | LEU | A | 291 | 59.319 | −10.400 | 6.867 | 1.00 | 33.61 | O |
| ATOM | 1600 | CB | LEU | A | 291 | 56.744 | −10.968 | 5.056 | 1.00 | 33.16 | C |
| ATOM | 1601 | CG | LEU | A | 291 | 56.064 | −10.759 | 6.415 | 1.00 | 35.62 | C |
| ATOM | 1602 | CD1 | LEU | A | 291 | 55.822 | −9.277 | 6.639 | 1.00 | 35.56 | C |
| ATOM | 1603 | CD2 | LEU | A | 291 | 54.751 | −11.524 | 6.468 | 1.00 | 36.46 | C |
| ATOM | 1604 | N | ALA | A | 292 | 59.957 | −11.694 | 5.150 | 1.00 | 37.49 | N |
| ATOM | 1605 | CA | ALA | A | 292 | 61.044 | −12.323 | 5.897 | 1.00 | 38.38 | C |
| ATOM | 1606 | C | ALA | A | 292 | 62.025 | −11.310 | 6.486 | 1.00 | 38.69 | C |
| ATOM | 1607 | O | ALA | A | 292 | 62.653 | −11.569 | 7.514 | 1.00 | 36.06 | O |
| ATOM | 1608 | CB | ALA | A | 292 | 61.792 | −13.308 | 5.001 | 1.00 | 39.60 | C |
| ATOM | 1609 | N | LYS | A | 293 | 62.145 | −10.155 | 5.840 | 1.00 | 39.29 | N |
| ATOM | 1610 | CA | LYS | A | 293 | 63.059 | −9.113 | 6.300 | 1.00 | 40.91 | C |
| ATOM | 1611 | C | LYS | A | 293 | 62.559 | −8.359 | 7.528 | 1.00 | 40.01 | C |
| ATOM | 1612 | O | LYS | A | 293 | 63.285 | −7.546 | 8.102 | 1.00 | 41.44 | O |
| ATOM | 1613 | CB | LYS | A | 293 | 63.320 | −8.112 | 5.172 | 1.00 | 43.98 | C |
| ATOM | 1614 | CG | LYS | A | 293 | 63.994 | −8.716 | 3.958 | 1.00 | 48.38 | C |
| ATOM | 1615 | CD | LYS | A | 293 | 64.268 | −7.663 | 2.899 | 1.00 | 51.31 | C |
| ATOM | 1616 | CE | LYS | A | 293 | 64.995 | −8.272 | 1.716 | 1.00 | 52.87 | C |
| ATOM | 1617 | NZ | LYS | A | 293 | 66.256 | −8.936 | 2.150 | 1.00 | 53.92 | N |
| ATOM | 1618 | N | THR | A | 294 | 61.323 | −8.627 | 7.931 | 1.00 | 36.01 | N |
| ATOM | 1619 | CA | THR | A | 294 | 60.744 | −7.950 | 9.083 | 1.00 | 31.40 | C |
| ATOM | 1620 | C | THR | A | 294 | 60.766 | −8.817 | 10.336 | 1.00 | 29.48 | C |
| ATOM | 1621 | O | THR | A | 294 | 60.321 | −8.395 | 11.402 | 1.00 | 28.18 | O |
| ATOM | 1622 | CB | THR | A | 294 | 59.289 | −7.549 | 8.797 | 1.00 | 34.96 | C |
| ATOM | 1623 | OG1 | THR | A | 294 | 58.469 | −8.723 | 8.721 | 1.00 | 31.27 | O |
| ATOM | 1624 | CG2 | THR | A | 294 | 59.204 | −6.808 | 7.473 | 1.00 | 37.20 | C |
| ATOM | 1625 | N | GLU | A | 295 | 61.300 | −10.026 | 10.209 | 1.00 | 26.38 | N |
| ATOM | 1626 | CA | GLU | A | 295 | 61.346 | −10.955 | 11.330 | 1.00 | 25.43 | C |
| ATOM | 1627 | C | GLU | A | 295 | 61.953 | −10.369 | 12.605 | 1.00 | 24.06 | C |
| ATOM | 1628 | O | GLU | A | 295 | 61.522 | −10.703 | 13.709 | 1.00 | 26.19 | O |
| ATOM | 1629 | CB | GLU | A | 295 | 62.110 | −12.214 | 10.927 | 1.00 | 29.11 | C |
| ATOM | 1630 | CG | GLU | A | 295 | 61.832 | −13.401 | 11.821 | 1.00 | 36.07 | C |
| ATOM | 1631 | CD | GLU | A | 295 | 62.641 | −14.613 | 11.424 | 1.00 | 40.84 | C |
| ATOM | 1632 | OE1 | GLU | A | 295 | 62.727 | −14.893 | 10.208 | 1.00 | 43.65 | O |
| ATOM | 1633 | OE2 | GLU | A | 295 | 63.183 | −15.284 | 12.328 | 1.00 | 44.13 | O |
| ATOM | 1634 | N | LYS | A | 296 | 62.946 | −9.494 | 12.456 | 1.00 | 22.44 | N |
| ATOM | 1635 | CA | LYS | A | 296 | 63.596 | −8.877 | 13.610 | 1.00 | 22.67 | C |
| ATOM | 1636 | C | LYS | A | 296 | 62.618 | −8.047 | 14.440 | 1.00 | 22.33 | C |
| ATOM | 1637 | O | LYS | A | 296 | 62.909 | −7.698 | 15.584 | 1.00 | 22.36 | O |
| ATOM | 1638 | CB | LYS | A | 296 | 64.757 | −7.979 | 13.162 | 1.00 | 25.65 | C |
| ATOM | 1639 | CG | LYS | A | 296 | 64.322 | −6.681 | 12.494 | 1.00 | 31.08 | C |
| ATOM | 1640 | CD | LYS | A | 296 | 65.509 | −5.773 | 12.176 | 1.00 | 35.06 | C |
| ATOM | 1641 | CE | LYS | A | 296 | 66.387 | −6.364 | 11.084 | 1.00 | 39.77 | C |
| ATOM | 1642 | NZ | LYS | A | 296 | 65.627 | −6.529 | 9.806 | 1.00 | 43.88 | N |
| ATOM | 1643 | N | TYR | A | 297 | 61.461 | −7.734 | 13.867 | 1.00 | 21.69 | N |
| ATOM | 1644 | CA | TYR | A | 297 | 60.454 | −6.931 | 14.563 | 1.00 | 20.68 | C |
| ATOM | 1645 | C | TYR | A | 297 | 59.276 | −7.724 | 15.132 | 1.00 | 21.43 | C |
| ATOM | 1646 | O | TYR | A | 297 | 58.479 | −7.181 | 15.896 | 1.00 | 21.62 | O |
| ATOM | 1647 | CB | TYR | A | 297 | 59.912 | −5.850 | 13.622 | 1.00 | 21.80 | C |
| ATOM | 1648 | CG | TYR | A | 297 | 60.967 | −4.871 | 13.165 | 1.00 | 22.04 | C |
| ATOM | 1649 | CD1 | TYR | A | 297 | 61.573 | −4.004 | 14.071 | 1.00 | 18.60 | C |
| ATOM | 1650 | CD2 | TYR | A | 297 | 61.380 | −4.831 | 11.833 | 1.00 | 20.46 | C |
| ATOM | 1651 | CE1 | TYR | A | 297 | 62.566 | −3.121 | 13.667 | 1.00 | 22.67 | C |
| ATOM | 1652 | CE2 | TYR | A | 297 | 62.376 | −3.948 | 11.418 | 1.00 | 26.04 | C |
| ATOM | 1653 | CZ | TYR | A | 297 | 62.962 | −3.098 | 12.344 | 1.00 | 22.57 | C |
| ATOM | 1654 | OH | TYR | A | 297 | 63.944 | −2.225 | 11.951 | 1.00 | 29.02 | O |
| ATOM | 1655 | N | TRP | A | 298 | 59.170 | −9.003 | 14.778 | 1.00 | 21.11 | N |
| ATOM | 1656 | CA | TRP | A | 298 | 58.059 | −9.820 | 15.253 | 1.00 | 22.41 | C |
| ATOM | 1657 | C | TRP | A | 298 | 57.943 | −9.888 | 16.767 | 1.00 | 24.35 | C |
| ATOM | 1658 | O | TRP | A | 298 | 56.840 | −9.824 | 17.308 | 1.00 | 22.97 | O |
| ATOM | 1659 | CB | TRP | A | 298 | 58.148 | −11.237 | 14.675 | 1.00 | 25.38 | C |
| ATOM | 1660 | CG | TRP | A | 298 | 58.063 | −11.276 | 13.175 | 1.00 | 26.95 | C |
| ATOM | 1661 | CD1 | TRP | A | 298 | 57.846 | −10.218 | 12.337 | 1.00 | 26.45 | C |
| ATOM | 1662 | CD2 | TRP | A | 298 | 58.211 | −12.429 | 12.337 | 1.00 | 30.19 | C |
| ATOM | 1663 | NE1 | TRP | A | 298 | 57.854 | −10.641 | 11.031 | 1.00 | 30.61 | N |
| ATOM | 1664 | CE2 | TRP | A | 298 | 58.076 | −11.993 | 11.001 | 1.00 | 31.26 | C |
| ATOM | 1665 | CE3 | TRP | A | 298 | 58.447 | −13.790 | 12.585 | 1.00 | 32.80 | C |
| ATOM | 1666 | CZ2 | TRP | A | 298 | 58.171 | −12.869 | 9.912 | 1.00 | 32.87 | C |
| ATOM | 1667 | CZ3 | TRP | A | 298 | 58.541 | −14.664 | 11.500 | 1.00 | 32.53 | C |
| ATOM | 1668 | CH2 | TRP | A | 298 | 58.403 | −14.196 | 10.182 | 1.00 | 34.66 | C |
| ATOM | 1669 | N | ASP | A | 299 | 59.070 | −10.018 | 17.457 | 1.00 | 21.39 | N |
| ATOM | 1670 | CA | ASP | A | 299 | 59.034 | −10.078 | 18.910 | 1.00 | 24.89 | C |
| ATOM | 1671 | C | ASP | A | 299 | 58.392 | −8.819 | 19.490 | 1.00 | 24.11 | C |
| ATOM | 1672 | O | ASP | A | 299 | 57.642 | −8.884 | 20.467 | 1.00 | 22.64 | O |
| ATOM | 1673 | CB | ASP | A | 299 | 60.443 | −10.219 | 19.484 | 1.00 | 27.59 | C |
| ATOM | 1674 | CG | ASP | A | 299 | 60.443 | −10.264 | 20.998 | 1.00 | 30.26 | C |
| ATOM | 1675 | OD1 | ASP | A | 299 | 60.035 | −11.299 | 21.561 | 1.00 | 29.61 | O |
| ATOM | 1676 | OD2 | ASP | A | 299 | 60.831 | −9.259 | 21.627 | 1.00 | 32.85 | O |
| ATOM | 1677 | N | GLY | A | 300 | 58.699 | −7.672 | 18.893 | 1.00 | 22.43 | N |
| ATOM | 1678 | CA | GLY | A | 300 | 58.135 | −6.423 | 19.379 | 1.00 | 18.10 | C |
| ATOM | 1679 | C | GLY | A | 300 | 56.644 | −6.354 | 19.106 | 1.00 | 18.45 | C |
| ATOM | 1680 | O | GLY | A | 300 | 55.882 | −5.779 | 19.882 | 1.00 | 18.47 | O |
| ATOM | 1681 | N | TRP | A | 301 | 56.219 | −6.946 | 17.997 | 1.00 | 18.75 | N |
| ATOM | 1682 | CA | TRP | A | 301 | 54.805 | −6.935 | 17.654 | 1.00 | 17.80 | C |
| ATOM | 1683 | C | TRP | A | 301 | 53.977 | −7.734 | 18.649 | 1.00 | 19.25 | C |
| ATOM | 1684 | O | TRP | A | 301 | 52.940 | −7.270 | 19.121 | 1.00 | 17.13 | O |
| ATOM | 1685 | CB | TRP | A | 301 | 54.563 | −7.555 | 16.276 | 1.00 | 18.96 | C |
| ATOM | 1686 | CG | TRP | A | 301 | 55.145 | −6.831 | 15.105 | 1.00 | 16.76 | C |
| ATOM | 1687 | CD1 | TRP | A | 301 | 55.761 | −5.610 | 15.104 | 1.00 | 18.10 | C |
| ATOM | 1688 | CD2 | TRP | A | 301 | 55.158 | −7.289 | 13.750 | 1.00 | 18.43 | C |
| ATOM | 1689 | NE1 | TRP | A | 301 | 56.157 | −5.283 | 13.829 | 1.00 | 17.03 | N |
| ATOM | 1690 | CE2 | TRP | A | 301 | 55.800 | −6.296 | 12.979 | 1.00 | 16.96 | C |
| ATOM | 1691 | CE3 | TRP | A | 301 | 54.687 | −8.447 | 13.111 | 1.00 | 18.26 | C |
| ATOM | 1692 | CZ2 | TRP | A | 301 | 55.987 | −6.424 | 11.601 | 1.00 | 16.89 | C |
| ATOM | 1693 | CZ3 | TRP | A | 301 | 54.873 | −8.573 | 11.742 | 1.00 | 18.21 | C |
| ATOM | 1694 | CH2 | TRP | A | 301 | 55.521 | −7.563 | 11.001 | 1.00 | 17.82 | C |
| ATOM | 1695 | N | PHE | A | 302 | 54.449 | −8.933 | 18.980 | 1.00 | 17.84 | N |
| ATOM | 1696 | CA | PHE | A | 302 | 53.672 | −9.825 | 19.832 | 1.00 | 19.57 | C |
| ATOM | 1697 | C | PHE | A | 302 | 53.969 | −9.976 | 21.318 | 1.00 | 18.64 | C |
| ATOM | 1698 | O | PHE | A | 302 | 53.086 | −10.402 | 22.071 | 1.00 | 18.42 | O |
| ATOM | 1699 | CB | PHE | A | 302 | 53.674 | −11.226 | 19.206 | 1.00 | 18.58 | C |
| ATOM | 1700 | CG | PHE | A | 302 | 53.310 | −11.236 | 17.746 | 1.00 | 20.81 | C |
| ATOM | 1701 | CD1 | PHE | A | 302 | 52.050 | −10.816 | 17.324 | 1.00 | 19.85 | C |
| ATOM | 1702 | CD2 | PHE | A | 302 | 54.229 | −11.663 | 16.791 | 1.00 | 21.87 | C |
| ATOM | 1703 | CE1 | PHE | A | 302 | 51.711 | −10.820 | 15.968 | 1.00 | 20.65 | C |
| ATOM | 1704 | CE2 | PHE | A | 302 | 53.900 | −11.671 | 15.429 | 1.00 | 20.49 | C |
| ATOM | 1705 | CZ | PHE | A | 302 | 52.638 | −11.248 | 15.019 | 1.00 | 17.49 | C |
| ATOM | 1706 | N | ARG | A | 303 | 55.181 | −9.655 | 21.755 | 1.00 | 20.68 | N |
| ATOM | 1707 | CA | ARG | A | 303 | 55.510 | −9.838 | 23.167 | 1.00 | 20.58 | C |
| ATOM | 1708 | C | ARG | A | 303 | 54.571 | −9.088 | 24.121 | 1.00 | 18.43 | C |
| ATOM | 1709 | O | ARG | A | 303 | 54.505 | −7.861 | 24.113 | 1.00 | 17.35 | O |
| ATOM | 1710 | CB | ARG | A | 303 | 56.970 | −9.447 | 23.430 | 1.00 | 25.09 | C |
| ATOM | 1711 | CG | ARG | A | 303 | 57.427 | −9.765 | 24.849 | 1.00 | 31.62 | C |
| ATOM | 1712 | CD | ARG | A | 303 | 58.927 | −9.570 | 25.041 | 1.00 | 39.26 | C |
| ATOM | 1713 | NE | ARG | A | 303 | 59.716 | −10.555 | 24.304 | 1.00 | 45.46 | N |
| ATOM | 1714 | CZ | ARG | A | 303 | 59.614 | −11.871 | 24.467 | 1.00 | 47.43 | C |
| ATOM | 1715 | NH1 | ARG | A | 303 | 58.752 | −12.372 | 25.344 | 1.00 | 50.74 | N |
| ATOM | 1716 | NH2 | ARG | A | 303 | 60.376 | −12.690 | 23.754 | 1.00 | 46.45 | N |
| ATOM | 1717 | N | GLY | A | 304 | 53.841 | −9.844 | 24.938 | 1.00 | 17.98 | N |
| ATOM | 1718 | CA | GLY | A | 304 | 52.913 | −9.248 | 25.890 | 1.00 | 17.00 | C |
| ATOM | 1719 | C | GLY | A | 304 | 51.639 | −8.707 | 25.257 | 1.00 | 18.08 | C |
| ATOM | 1720 | O | GLY | A | 304 | 50.843 | −8.035 | 25.916 | 1.00 | 14.62 | O |
| ATOM | 1721 | N | LEU | A | 305 | 51.429 | −9.017 | 23.981 | 1.00 | 17.30 | N |
| ATOM | 1722 | CA | LEU | A | 305 | 50.256 | −8.522 | 23.260 | 1.00 | 15.72 | C |
| ATOM | 1723 | C | LEU | A | 305 | 48.911 | −8.904 | 23.871 | 1.00 | 13.98 | C |
| ATOM | 1724 | O | LEU | A | 305 | 47.989 | −8.086 | 23.909 | 1.00 | 14.00 | O |
| ATOM | 1725 | CB | LEU | A | 305 | 50.291 | −8.995 | 21.805 | 1.00 | 16.60 | C |
| ATOM | 1726 | CG | LEU | A | 305 | 49.287 | −8.337 | 20.848 | 1.00 | 19.49 | C |
| ATOM | 1727 | CD1 | LEU | A | 305 | 49.673 | −6.880 | 20.644 | 1.00 | 18.08 | C |
| ATOM | 1728 | CD2 | LEU | A | 305 | 49.296 | −9.056 | 19.507 | 1.00 | 18.15 | C |
| ATOM | 1729 | N | SER | A | 306 | 48.777 | −10.139 | 24.336 | 1.00 | 15.08 | N |
| ATOM | 1730 | CA | SER | A | 306 | 47.498 | −10.545 | 24.907 | 1.00 | 14.89 | C |
| ATOM | 1731 | C | SER | A | 306 | 47.103 | −9.694 | 26.110 | 1.00 | 13.85 | C |
| ATOM | 1732 | O | SER | A | 306 | 45.992 | −9.170 | 26.160 | 1.00 | 16.59 | O |
| ATOM | 1733 | CB | SER | A | 306 | 47.520 | −12.028 | 25.260 | 1.00 | 14.63 | C |
| ATOM | 1734 | OG | SER | A | 306 | 47.461 | −12.804 | 24.070 | 1.00 | 11.99 | O |
| ATOM | 1735 | N | ASN | A | 307 | 48.010 | −9.527 | 27.065 | 1.00 | 13.22 | N |
| ATOM | 1736 | CA | ASN | A | 307 | 47.693 | −8.715 | 28.238 | 1.00 | 15.96 | C |
| ATOM | 1737 | C | ASN | A | 307 | 47.587 | −7.236 | 27.870 | 1.00 | 15.89 | C |
| ATOM | 1738 | O | ASN | A | 307 | 46.845 | −6.483 | 28.497 | 1.00 | 14.67 | O |
| ATOM | 1739 | CB | ASN | A | 307 | 48.742 | −8.944 | 29.330 | 1.00 | 17.02 | C |
| ATOM | 1740 | CG | ASN | A | 307 | 48.562 | −10.292 | 30.024 | 1.00 | 20.28 | C |
| ATOM | 1741 | OD1 | ASN | A | 307 | 49.485 | −10.827 | 30.640 | 1.00 | 24.97 | O |
| ATOM | 1742 | ND2 | ASN | A | 307 | 47.357 | −10.837 | 29.929 | 1.00 | 17.79 | N |
| ATOM | 1743 | N | LEU | A | 308 | 48.320 | −6.831 | 26.838 | 1.00 | 16.03 | N |
| ATOM | 1744 | CA | LEU | A | 308 | 48.289 | −5.448 | 26.377 | 1.00 | 16.90 | C |
| ATOM | 1745 | C | LEU | A | 308 | 46.878 | −5.163 | 25.846 | 1.00 | 17.30 | C |
| ATOM | 1746 | O | LEU | A | 308 | 46.285 | −4.123 | 26.128 | 1.00 | 14.82 | O |
| ATOM | 1747 | CB | LEU | A | 308 | 49.328 | −5.264 | 25.271 | 1.00 | 21.81 | C |
| ATOM | 1748 | CG | LEU | A | 308 | 49.881 | −3.862 | 25.041 | 1.00 | 26.54 | C |
| ATOM | 1749 | CD1 | LEU | A | 308 | 50.516 | −3.344 | 26.327 | 1.00 | 27.16 | C |
| ATOM | 1750 | CD2 | LEU | A | 308 | 50.915 | −3.909 | 23.913 | 1.00 | 25.12 | C |
| ATOM | 1751 | N | PHE | A | 309 | 46.358 | −6.112 | 25.073 | 1.00 | 14.40 | N |
| ATOM | 1752 | CA | PHE | A | 309 | 45.017 | −6.054 | 24.493 | 1.00 | 16.51 | C |
| ATOM | 1753 | C | PHE | A | 309 | 43.981 | −5.935 | 25.620 | 1.00 | 15.97 | C |
| ATOM | 1754 | O | PHE | A | 309 | 43.146 | −5.024 | 25.630 | 1.00 | 16.32 | O |
| ATOM | 1755 | CB | PHE | A | 309 | 44.795 | −7.346 | 23.679 | 1.00 | 13.23 | C |
| ATOM | 1756 | CG | PHE | A | 309 | 43.366 | −7.593 | 23.250 | 1.00 | 17.71 | C |
| ATOM | 1757 | CD1 | PHE | A | 309 | 42.787 | −6.856 | 22.220 | 1.00 | 14.13 | C |
| ATOM | 1758 | CD2 | PHE | A | 309 | 42.628 | −8.625 | 23.829 | 1.00 | 11.86 | C |
| ATOM | 1759 | CE1 | PHE | A | 309 | 41.500 | −7.150 | 21.765 | 1.00 | 15.58 | C |
| ATOM | 1760 | CE2 | PHE | A | 309 | 41.338 | −8.929 | 23.385 | 1.00 | 13.49 | C |
| ATOM | 1761 | CZ | PHE | A | 309 | 40.773 | −8.192 | 22.349 | 1.00 | 15.08 | C |
| ATOM | 1762 | N | LEU | A | 310 | 44.055 | −6.852 | 26.580 | 1.00 | 16.15 | N |
| ATOM | 1763 | CA | LEU | A | 310 | 43.128 | −6.874 | 27.706 | 1.00 | 16.27 | C |
| ATOM | 1764 | C | LEU | A | 310 | 43.110 | −5.585 | 28.530 | 1.00 | 19.29 | C |
| ATOM | 1765 | O | LEU | A | 310 | 42.066 | −5.201 | 29.051 | 1.00 | 20.70 | O |
| ATOM | 1766 | CB | LEU | A | 310 | 43.457 | −8.058 | 28.625 | 1.00 | 17.40 | C |
| ATOM | 1767 | CG | LEU | A | 310 | 43.295 | −9.455 | 28.008 | 1.00 | 15.86 | C |
| ATOM | 1768 | CD1 | LEU | A | 310 | 43.771 | −10.521 | 28.989 | 1.00 | 16.09 | C |
| ATOM | 1769 | CD2 | LEU | A | 310 | 41.837 | −9.674 | 27.631 | 1.00 | 12.13 | C |
| ATOM | 1770 | N | SER | A | 311 | 44.256 | −4.918 | 28.636 | 1.00 | 17.31 | N |
| ATOM | 1771 | CA | SER | A | 311 | 44.359 | −3.693 | 29.421 | 1.00 | 21.24 | C |
| ATOM | 1772 | C | SER | A | 311 | 43.758 | −2.446 | 28.772 | 1.00 | 19.92 | C |
| ATOM | 1773 | O | SER | A | 311 | 43.470 | −1.476 | 29.471 | 1.00 | 19.51 | O |
| ATOM | 1774 | CB | SER | A | 311 | 45.821 | −3.409 | 29.766 | 1.00 | 22.07 | C |
| ATOM | 1775 | OG | SER | A | 311 | 46.511 | −2.915 | 28.631 | 1.00 | 34.54 | O |
| ATOM | 1776 | N | CYS | A | 312 | 43.568 | −2.456 | 27.452 | 1.00 | 17.20 | N |
| ATOM | 1777 | CA | CYS | A | 312 | 42.993 | −1.287 | 26.772 | 1.00 | 17.81 | C |
| ATOM | 1778 | C | CYS | A | 312 | 41.593 | −1.005 | 27.324 | 1.00 | 15.71 | C |
| ATOM | 1779 | O | CYS | A | 312 | 40.718 | −1.865 | 27.279 | 1.00 | 15.56 | O |
| ATOM | 1780 | CB | CYS | A | 312 | 42.941 | −1.520 | 25.256 | 1.00 | 17.06 | C |
| ATOM | 1781 | SG | CYS | A | 312 | 44.589 | −1.677 | 24.485 | 1.00 | 18.46 | S |
| ATOM | 1782 | N | PRO | A | 313 | 41.368 | 0.217 | 27.843 | 1.00 | 20.34 | N |
| ATOM | 1783 | CA | PRO | A | 313 | 40.086 | 0.634 | 28.424 | 1.00 | 19.28 | C |
| ATOM | 1784 | C | PRO | A | 313 | 38.930 | 0.947 | 27.473 | 1.00 | 20.89 | C |
| ATOM | 1785 | O | PRO | A | 313 | 38.286 | 1.992 | 27.590 | 1.00 | 20.46 | O |
| ATOM | 1786 | CB | PRO | A | 313 | 40.484 | 1.836 | 29.275 | 1.00 | 19.81 | C |
| ATOM | 1787 | CG | PRO | A | 313 | 41.517 | 2.499 | 28.414 | 1.00 | 20.49 | C |
| ATOM | 1788 | CD | PRO | A | 313 | 42.356 | 1.315 | 27.917 | 1.00 | 22.23 | C |
| ATOM | 1789 | N | ILE | A | 314 | 38.659 | 0.033 | 26.546 | 1.00 | 19.62 | N |
| ATOM | 1790 | CA | ILE | A | 314 | 37.566 | 0.186 | 25.584 | 1.00 | 16.87 | C |
| ATOM | 1791 | C | ILE | A | 314 | 37.015 | −1.197 | 25.285 | 1.00 | 15.62 | C |
| ATOM | 1792 | O | ILE | A | 314 | 37.713 | −2.193 | 25.476 | 1.00 | 17.16 | O |
| ATOM | 1793 | CB | ILE | A | 314 | 38.054 | 0.754 | 24.237 | 1.00 | 18.79 | C |
| ATOM | 1794 | CG1 | ILE | A | 314 | 39.250 | −0.077 | 23.744 | 1.00 | 22.01 | C |
| ATOM | 1795 | CG2 | ILE | A | 314 | 38.411 | 2.223 | 24.380 | 1.00 | 23.11 | C |
| ATOM | 1796 | CD1 | ILE | A | 314 | 39.733 | 0.281 | 22.383 | 1.00 | 25.58 | C |
| ATOM | 1797 | N | PRO | A | 315 | 35.761 | −1.281 | 24.809 | 1.00 | 16.25 | N |
| ATOM | 1798 | CA | PRO | A | 315 | 35.175 | −2.589 | 24.487 | 1.00 | 15.63 | C |
| ATOM | 1799 | C | PRO | A | 315 | 36.060 | −3.251 | 23.429 | 1.00 | 15.25 | C |
| ATOM | 1800 | O | PRO | A | 315 | 36.565 | −2.575 | 22.533 | 1.00 | 10.57 | O |
| ATOM | 1801 | CB | PRO | A | 315 | 33.789 | −2.223 | 23.943 | 1.00 | 16.28 | C |
| ATOM | 1802 | CG | PRO | A | 315 | 33.448 | −0.985 | 24.729 | 1.00 | 17.96 | C |
| ATOM | 1803 | CD | PRO | A | 315 | 34.755 | −0.209 | 24.670 | 1.00 | 14.46 | C |
| ATOM | 1804 | N | LYS | A | 316 | 36.223 | −4.567 | 23.517 | 1.00 | 13.08 | N |
| ATOM | 1805 | CA | LYS | A | 316 | 37.098 | −5.288 | 22.599 | 1.00 | 16.26 | C |
| ATOM | 1806 | C | LYS | A | 316 | 36.459 | −6.502 | 21.936 | 1.00 | 16.74 | C |
| ATOM | 1807 | O | LYS | A | 316 | 35.573 | −7.148 | 22.499 | 1.00 | 14.46 | O |
| ATOM | 1808 | CB | LYS | A | 316 | 38.356 | −5.736 | 23.355 | 1.00 | 15.66 | C |
| ATOM | 1809 | CG | LYS | A | 316 | 39.205 | −4.579 | 23.877 | 1.00 | 17.61 | C |
| ATOM | 1810 | CD | LYS | A | 316 | 40.269 | −5.046 | 24.858 | 1.00 | 14.72 | C |
| ATOM | 1811 | CE | LYS | A | 316 | 39.676 | −5.417 | 26.210 | 1.00 | 14.13 | C |
| ATOM | 1812 | NZ | LYS | A | 316 | 39.093 | −4.232 | 26.915 | 1.00 | 14.13 | N |
| ATOM | 1813 | N | LEU | A | 317 | 36.943 | −6.821 | 20.742 | 1.00 | 16.43 | N |
| ATOM | 1814 | CA | LEU | A | 317 | 36.431 | −7.958 | 19.994 | 1.00 | 15.23 | C |
| ATOM | 1815 | C | LEU | A | 317 | 37.565 | −8.813 | 19.449 | 1.00 | 15.38 | C |
| ATOM | 1816 | O | LEU | A | 317 | 38.504 | −8.299 | 18.846 | 1.00 | 12.56 | O |
| ATOM | 1817 | CB | LEU | A | 317 | 35.574 | −7.472 | 18.823 | 1.00 | 16.03 | C |
| ATOM | 1818 | CG | LEU | A | 317 | 35.024 | −8.525 | 17.851 | 1.00 | 15.85 | C |
| ATOM | 1819 | CD1 | LEU | A | 317 | 34.112 | −9.505 | 18.611 | 1.00 | 17.87 | C |
| ATOM | 1820 | CD2 | LEU | A | 317 | 34.247 | −7.824 | 16.731 | 1.00 | 16.71 | C |
| ATOM | 1821 | N | LEU | A | 318 | 37.488 | −10.117 | 19.676 | 1.00 | 12.50 | N |
| ATOM | 1822 | CA | LEU | A | 318 | 38.492 | −11.018 | 19.126 | 1.00 | 12.30 | C |
| ATOM | 1823 | C | LEU | A | 318 | 37.761 | −11.975 | 18.187 | 1.00 | 14.34 | C |
| ATOM | 1824 | O | LEU | A | 318 | 36.798 | −12.639 | 18.587 | 1.00 | 15.80 | O |
| ATOM | 1825 | CB | LEU | A | 318 | 39.184 | −11.835 | 20.213 | 1.00 | 12.69 | C |
| ATOM | 1826 | CG | LEU | A | 318 | 40.092 | −12.935 | 19.634 | 1.00 | 9.89 | C |
| ATOM | 1827 | CD1 | LEU | A | 318 | 41.214 | −12.307 | 18.806 | 1.00 | 12.40 | C |
| ATOM | 1828 | CD2 | LEU | A | 318 | 40.690 | −13.752 | 20.781 | 1.00 | 16.37 | C |
| ATOM | 1829 | N | LEU | A | 319 | 38.210 | −12.032 | 16.942 | 1.00 | 13.47 | N |
| ATOM | 1830 | CA | LEU | A | 319 | 37.612 | −12.927 | 15.965 | 1.00 | 17.61 | C |
| ATOM | 1831 | C | LEU | A | 319 | 38.610 | −14.034 | 15.644 | 1.00 | 16.90 | C |
| ATOM | 1832 | O | LEU | A | 319 | 39.755 | −13.764 | 15.272 | 1.00 | 15.23 | O |
| ATOM | 1833 | CB | LEU | A | 319 | 37.256 | −12.164 | 14.686 | 1.00 | 16.06 | C |
| ATOM | 1834 | CG | LEU | A | 319 | 36.227 | −11.035 | 14.853 | 1.00 | 18.75 | C |
| ATOM | 1835 | CD1 | LEU | A | 319 | 35.986 | −10.365 | 13.498 | 1.00 | 18.62 | C |
| ATOM | 1836 | CD2 | LEU | A | 319 | 34.929 | −11.585 | 15.401 | 1.00 | 16.91 | C |
| ATOM | 1837 | N | LEU | A | 320 | 38.180 | −15.280 | 15.808 | 1.00 | 17.19 | N |
| ATOM | 1838 | CA | LEU | A | 320 | 39.045 | −16.417 | 15.509 | 1.00 | 17.49 | C |
| ATOM | 1839 | C | LEU | A | 320 | 38.565 | −17.095 | 14.236 | 1.00 | 20.03 | C |
| ATOM | 1840 | O | LEU | A | 320 | 37.364 | −17.270 | 14.034 | 1.00 | 24.46 | O |
| ATOM | 1841 | CB | LEU | A | 320 | 39.036 | −17.429 | 16.661 | 1.00 | 16.67 | C |
| ATOM | 1842 | CG | LEU | A | 320 | 39.623 | −16.933 | 17.989 | 1.00 | 18.01 | C |
| ATOM | 1843 | CD1 | LEU | A | 320 | 39.543 | −18.043 | 19.023 | 1.00 | 21.52 | C |
| ATOM | 1844 | CD2 | LEU | A | 320 | 41.071 | −16.495 | 17.782 | 1.00 | 21.66 | C |
| ATOM | 1845 | N | ALA | A | 321 | 39.507 | −17.459 | 13.374 | 1.00 | 20.78 | N |
| ATOM | 1846 | CA | ALA | A | 321 | 39.188 | −18.134 | 12.120 | 1.00 | 23.16 | C |
| ATOM | 1847 | C | ALA | A | 321 | 40.257 | −19.197 | 11.897 | 1.00 | 25.25 | C |
| ATOM | 1848 | O | ALA | A | 321 | 40.015 | −20.381 | 12.119 | 1.00 | 27.55 | O |
| ATOM | 1849 | CB | ALA | A | 321 | 39.178 | −17.137 | 10.967 | 1.00 | 23.05 | C |
| ATOM | 1850 | N | GLY | A | 322 | 41.441 | −18.763 | 11.473 | 1.00 | 24.91 | N |
| ATOM | 1851 | CA | GLY | A | 322 | 42.541 | −19.686 | 11.256 | 1.00 | 24.48 | C |
| ATOM | 1852 | C | GLY | A | 322 | 43.115 | −20.158 | 12.584 | 1.00 | 26.57 | C |
| ATOM | 1853 | O | GLY | A | 322 | 43.567 | −21.296 | 12.705 | 1.00 | 30.22 | O |
| ATOM | 1854 | N | VAL | A | 323 | 43.096 | −19.285 | 13.588 | 1.00 | 21.50 | N |
| ATOM | 1855 | CA | VAL | A | 323 | 43.606 | −19.637 | 14.908 | 1.00 | 19.52 | C |
| ATOM | 1856 | C | VAL | A | 323 | 42.502 | −20.397 | 15.630 | 1.00 | 19.57 | C |
| ATOM | 1857 | O | VAL | A | 323 | 41.379 | −19.908 | 15.765 | 1.00 | 18.58 | O |
| ATOM | 1858 | CB | VAL | A | 323 | 43.993 | −18.379 | 15.711 | 1.00 | 18.00 | C |
| ATOM | 1859 | CG1 | VAL | A | 323 | 44.519 | −18.778 | 17.090 | 1.00 | 15.48 | C |
| ATOM | 1860 | CG2 | VAL | A | 323 | 45.047 | −17.594 | 14.952 | 1.00 | 17.42 | C |
| ATOM | 1861 | N | ASP | A | 324 | 42.823 | −21.594 | 16.103 | 1.00 | 21.52 | N |
| ATOM | 1862 | CA | ASP | A | 324 | 41.831 | −22.437 | 16.764 | 1.00 | 21.06 | C |
| ATOM | 1863 | C | ASP | A | 324 | 41.795 | −22.399 | 18.286 | 1.00 | 20.53 | C |
| ATOM | 1864 | O | ASP | A | 324 | 40.938 | −23.038 | 18.895 | 1.00 | 24.13 | O |
| ATOM | 1865 | CB | ASP | A | 324 | 42.012 | −23.886 | 16.292 | 1.00 | 26.72 | C |
| ATOM | 1866 | CG | ASP | A | 324 | 43.372 | −24.460 | 16.669 | 1.00 | 31.50 | C |
| ATOM | 1867 | OD1 | ASP | A | 324 | 44.343 | −23.680 | 16.771 | 1.00 | 30.04 | O |
| ATOM | 1868 | OD2 | ASP | A | 324 | 43.472 | −25.695 | 16.850 | 1.00 | 31.44 | O |
| ATOM | 1869 | N | ARG | A | 325 | 42.702 | −21.654 | 18.910 | 1.00 | 16.84 | N |
| ATOM | 1870 | CA | ARG | A | 325 | 42.721 | −21.593 | 20.371 | 1.00 | 17.80 | C |
| ATOM | 1871 | C | ARG | A | 325 | 43.188 | −20.266 | 20.940 | 1.00 | 15.60 | C |
| ATOM | 1872 | O | ARG | A | 325 | 43.970 | −19.546 | 20.320 | 1.00 | 15.36 | O |
| ATOM | 1873 | CB | ARG | A | 325 | 43.672 | −22.648 | 20.951 | 1.00 | 18.58 | C |
| ATOM | 1874 | CG | ARG | A | 325 | 43.521 | −24.062 | 20.451 | 1.00 | 27.24 | C |
| ATOM | 1875 | CD | ARG | A | 325 | 44.719 | −24.853 | 20.954 | 1.00 | 27.71 | C |
| ATOM | 1876 | NE | ARG | A | 325 | 44.629 | −26.281 | 20.685 | 1.00 | 32.67 | N |
| ATOM | 1877 | CZ | ARG | A | 325 | 45.529 | −27.162 | 21.108 | 1.00 | 33.73 | C |
| ATOM | 1878 | NH1 | ARG | A | 325 | 46.578 | −26.747 | 21.813 | 1.00 | 29.24 | N |
| ATOM | 1879 | NH2 | ARG | A | 325 | 45.384 | −28.450 | 20.830 | 1.00 | 36.08 | N |
| ATOM | 1880 | N | LEU | A | 326 | 42.728 | −19.968 | 22.151 | 1.00 | 16.47 | N |
| ATOM | 1881 | CA | LEU | A | 326 | 43.157 | −18.764 | 22.859 | 1.00 | 16.66 | C |
| ATOM | 1882 | C | LEU | A | 326 | 44.406 | −19.211 | 23.608 | 1.00 | 18.54 | C |
| ATOM | 1883 | O | LEU | A | 326 | 44.633 | −20.412 | 23.757 | 1.00 | 17.93 | O |
| ATOM | 1884 | CB | LEU | A | 326 | 42.119 | −18.337 | 23.896 | 1.00 | 17.67 | C |
| ATOM | 1885 | CG | LEU | A | 326 | 40.726 | −17.924 | 23.427 | 1.00 | 20.49 | C |
| ATOM | 1886 | CD1 | LEU | A | 326 | 39.839 | −17.692 | 24.655 | 1.00 | 17.54 | C |
| ATOM | 1887 | CD2 | LEU | A | 326 | 40.830 | −16.663 | 22.566 | 1.00 | 18.50 | C |
| ATOM | 1888 | N | ASP | A | 327 | 45.224 | −18.268 | 24.068 | 1.00 | 14.52 | N |
| ATOM | 1889 | CA | ASP | A | 327 | 46.392 | −18.654 | 24.845 | 1.00 | 16.63 | C |
| ATOM | 1890 | C | ASP | A | 327 | 45.947 | −18.569 | 26.296 | 1.00 | 17.05 | C |
| ATOM | 1891 | O | ASP | A | 327 | 44.779 | −18.281 | 26.570 | 1.00 | 18.09 | O |
| ATOM | 1892 | CB | ASP | A | 327 | 47.599 | −17.731 | 24.577 | 1.00 | 14.93 | C |
| ATOM | 1893 | CG | ASP | A | 327 | 47.263 | −16.252 | 24.688 | 1.00 | 15.54 | C |
| ATOM | 1894 | OD1 | ASP | A | 327 | 46.221 | −15.911 | 25.289 | 1.00 | 17.45 | O |
| ATOM | 1895 | OD2 | ASP | A | 327 | 48.063 | −15.429 | 24.176 | 1.00 | 14.74 | O |
| ATOM | 1896 | N | LYS | A | 328 | 46.849 | −18.835 | 27.228 | 1.00 | 18.65 | N |
| ATOM | 1897 | CA | LYS | A | 328 | 46.487 | −18.783 | 28.640 | 1.00 | 20.74 | C |
| ATOM | 1898 | C | LYS | A | 328 | 46.016 | −17.397 | 29.060 | 1.00 | 16.97 | C |
| ATOM | 1899 | O | LYS | A | 328 | 45.026 | −17.273 | 29.772 | 1.00 | 16.78 | O |
| ATOM | 1900 | CB | LYS | A | 328 | 47.672 | −19.188 | 29.513 | 1.00 | 26.69 | C |
| ATOM | 1901 | CG | LYS | A | 328 | 48.199 | −20.574 | 29.224 | 1.00 | 35.24 | C |
| ATOM | 1902 | CD | LYS | A | 328 | 49.450 | −20.865 | 30.044 | 1.00 | 40.47 | C |
| ATOM | 1903 | CE | LYS | A | 328 | 49.969 | −22.257 | 29.746 | 1.00 | 43.81 | C |
| ATOM | 1904 | NZ | LYS | A | 328 | 48.911 | −23.280 | 29.982 | 1.00 | 44.28 | N |
| ATOM | 1905 | N | ASP | A | 329 | 46.719 | −16.354 | 28.623 | 1.00 | 17.24 | N |
| ATOM | 1906 | CA | ASP | A | 329 | 46.333 | −14.992 | 29.005 | 1.00 | 16.77 | C |
| ATOM | 1907 | C | ASP | A | 329 | 44.912 | −14.609 | 28.582 | 1.00 | 15.98 | C |
| ATOM | 1908 | O | ASP | A | 329 | 44.160 | −14.040 | 29.372 | 1.00 | 16.57 | O |
| ATOM | 1909 | CB | ASP | A | 329 | 47.314 | −13.955 | 28.437 | 1.00 | 16.47 | C |
| ATOM | 1910 | CG | ASP | A | 329 | 48.701 | −14.051 | 29.060 | 1.00 | 24.79 | C |
| ATOM | 1911 | OD1 | ASP | A | 329 | 48.800 | −14.447 | 30.240 | 1.00 | 18.21 | O |
| ATOM | 1912 | OD2 | ASP | A | 329 | 49.691 | −13.718 | 28.369 | 1.00 | 24.60 | O |
| ATOM | 1913 | N | LEU | A | 330 | 44.553 | −14.907 | 27.335 | 1.00 | 14.92 | N |
| ATOM | 1914 | CA | LEU | A | 330 | 43.221 | −14.576 | 26.823 | 1.00 | 16.31 | C |
| ATOM | 1915 | C | LEU | A | 330 | 42.137 | −15.458 | 27.422 | 1.00 | 16.65 | C |
| ATOM | 1916 | O | LEU | A | 330 | 40.997 | −15.027 | 27.604 | 1.00 | 14.89 | O |
| ATOM | 1917 | CB | LEU | A | 330 | 43.199 | −14.695 | 25.297 | 1.00 | 13.16 | C |
| ATOM | 1918 | CG | LEU | A | 330 | 44.133 | −13.704 | 24.588 | 1.00 | 17.17 | C |
| ATOM | 1919 | CD1 | LEU | A | 330 | 44.118 | −13.948 | 23.074 | 1.00 | 19.51 | C |
| ATOM | 1920 | CD2 | LEU | A | 330 | 43.683 | −12.283 | 24.918 | 1.00 | 13.64 | C |
| ATOM | 1921 | N | THR | A | 331 | 42.484 | −16.705 | 27.714 | 1.00 | 15.54 | N |
| ATOM | 1922 | CA | THR | A | 331 | 41.514 | −17.610 | 28.308 | 1.00 | 17.21 | C |
| ATOM | 1923 | C | THR | A | 331 | 41.089 | −17.011 | 29.645 | 1.00 | 14.23 | C |
| ATOM | 1924 | O | THR | A | 331 | 39.898 | −16.942 | 29.953 | 1.00 | 13.58 | O |
| ATOM | 1925 | CB | THR | A | 331 | 42.123 | −19.004 | 28.512 | 1.00 | 15.02 | C |
| ATOM | 1926 | OG1 | THR | A | 331 | 42.482 | −19.537 | 27.230 | 1.00 | 16.74 | O |
| ATOM | 1927 | CG2 | THR | A | 331 | 41.124 | −19.936 | 29.188 | 1.00 | 17.29 | C |
| ATOM | 1928 | N | ILE | A | 332 | 42.073 | −16.547 | 30.415 | 1.00 | 13.85 | N |
| ATOM | 1929 | CA | ILE | A | 332 | 41.823 | −15.924 | 31.717 | 1.00 | 16.67 | C |
| ATOM | 1930 | C | ILE | A | 332 | 40.996 | −14.655 | 31.521 | 1.00 | 17.49 | C |
| ATOM | 1931 | O | ILE | A | 332 | 40.014 | −14.423 | 32.227 | 1.00 | 18.16 | O |
| ATOM | 1932 | CB | ILE | A | 332 | 43.167 | −15.570 | 32.425 | 1.00 | 17.96 | C |
| ATOM | 1933 | CG1 | ILE | A | 332 | 43.815 | −16.849 | 32.966 | 1.00 | 21.47 | C |
| ATOM | 1934 | CG2 | ILE | A | 332 | 42.929 | −14.574 | 33.560 | 1.00 | 20.50 | C |
| ATOM | 1935 | CD1 | ILE | A | 332 | 45.262 | −16.675 | 33.397 | 1.00 | 26.74 | C |
| ATOM | 1936 | N | GLY | A | 333 | 41.397 | −13.837 | 30.552 | 1.00 | 19.23 | N |
| ATOM | 1937 | CA | GLY | A | 333 | 40.669 | −12.612 | 30.277 | 1.00 | 17.02 | C |
| ATOM | 1938 | C | GLY | A | 333 | 39.233 | −12.894 | 29.875 | 1.00 | 17.10 | C |
| ATOM | 1939 | O | GLY | A | 333 | 38.316 | −12.155 | 30.248 | 1.00 | 14.76 | O |
| ATOM | 1940 | N | GLN | A | 334 | 39.029 | −13.959 | 29.103 | 1.00 | 14.27 | N |
| ATOM | 1941 | CA | GLN | A | 334 | 37.684 | −14.320 | 28.672 | 1.00 | 16.48 | C |
| ATOM | 1942 | C | GLN | A | 334 | 36.835 | −14.716 | 29.884 | 1.00 | 18.02 | C |
| ATOM | 1943 | O | GLN | A | 334 | 35.667 | −14.332 | 29.984 | 1.00 | 16.35 | O |
| ATOM | 1944 | CB | GLN | A | 334 | 37.739 | −15.473 | 27.659 | 1.00 | 17.53 | C |
| ATOM | 1945 | CG | GLN | A | 334 | 36.377 | −15.952 | 27.135 | 1.00 | 17.24 | C |
| ATOM | 1946 | CD | GLN | A | 334 | 35.543 | −14.843 | 26.491 | 1.00 | 23.43 | C |
| ATOM | 1947 | OE1 | GLN | A | 334 | 36.071 | −13.954 | 25.804 | 1.00 | 21.21 | O |
| ATOM | 1948 | NE2 | GLN | A | 334 | 34.229 | −14.907 | 26.690 | 1.00 | 15.69 | N |
| ATOM | 1949 | N | MET | A | 335 | 37.422 | −15.473 | 30.807 | 1.00 | 20.35 | N |
| ATOM | 1950 | CA | MET | A | 335 | 36.693 | −15.900 | 32.006 | 1.00 | 22.49 | C |
| ATOM | 1951 | C | MET | A | 335 | 36.311 | −14.682 | 32.838 | 1.00 | 22.40 | C |
| ATOM | 1952 | O | MET | A | 335 | 35.336 | −14.709 | 33.588 | 1.00 | 20.23 | O |
| ATOM | 1953 | CB | MET | A | 335 | 37.552 | −16.843 | 32.850 | 1.00 | 28.77 | C |
| ATOM | 1954 | CG | MET | A | 335 | 37.824 | −18.182 | 32.190 | 1.00 | 37.48 | C |
| ATOM | 1955 | SD | MET | A | 335 | 36.311 | −19.162 | 31.959 | 1.00 | 49.94 | S |
| ATOM | 1956 | CE | MET | A | 335 | 35.790 | −18.658 | 30.298 | 1.00 | 39.66 | C |
| ATOM | 1957 | N | GLN | A | 336 | 37.088 | −13.612 | 32.691 | 1.00 | 21.53 | N |
| ATOM | 1958 | CA | GLN | A | 336 | 36.842 | −12.369 | 33.408 | 1.00 | 20.95 | C |
| ATOM | 1959 | C | GLN | A | 336 | 35.901 | −11.455 | 32.625 | 1.00 | 20.52 | C |
| ATOM | 1960 | O | GLN | A | 336 | 35.581 | −10.358 | 33.075 | 1.00 | 18.72 | O |
| ATOM | 1961 | CB | GLN | A | 336 | 38.175 | −11.659 | 33.682 | 1.00 | 19.41 | C |
| ATOM | 1962 | CG | GLN | A | 336 | 39.060 | −12.424 | 34.665 | 1.00 | 21.77 | C |
| ATOM | 1963 | CD | GLN | A | 336 | 40.461 | −11.847 | 34.790 | 1.00 | 22.47 | C |
| ATOM | 1964 | OE1 | GLN | A | 336 | 41.126 | −12.037 | 35.802 | 1.00 | 29.72 | O |
| ATOM | 1965 | NE2 | GLN | A | 336 | 40.916 | −11.157 | 33.758 | 1.00 | 19.94 | N |
| ATOM | 1966 | N | GLY | A | 337 | 35.459 | −11.919 | 31.457 | 1.00 | 20.84 | N |
| ATOM | 1967 | CA | GLY | A | 337 | 34.545 | −11.143 | 30.628 | 1.00 | 18.59 | C |
| ATOM | 1968 | C | GLY | A | 337 | 35.110 | −9.833 | 30.096 | 1.00 | 21.02 | C |
| ATOM | 1969 | O | GLY | A | 337 | 34.378 | −8.856 | 29.949 | 1.00 | 20.09 | O |
| ATOM | 1970 | N | LYS | A | 338 | 36.401 | −9.815 | 29.779 | 1.00 | 18.31 | N |
| ATOM | 1971 | CA | LYS | A | 338 | 37.048 | −8.599 | 29.301 | 1.00 | 19.59 | C |
| ATOM | 1972 | C | LYS | A | 338 | 36.948 | −8.308 | 27.812 | 1.00 | 19.95 | C |
| ATOM | 1973 | O | LYS | A | 338 | 37.349 | −7.232 | 27.372 | 1.00 | 20.04 | O |
| ATOM | 1974 | CB | LYS | A | 338 | 38.519 | −8.593 | 29.724 | 1.00 | 19.56 | C |
| ATOM | 1975 | CG | LYS | A | 338 | 38.700 | −8.467 | 31.223 | 1.00 | 22.70 | C |
| ATOM | 1976 | CD | LYS | A | 338 | 40.163 | −8.513 | 31.645 | 1.00 | 24.39 | C |
| ATOM | 1977 | CE | LYS | A | 338 | 40.283 | −8.228 | 33.138 | 1.00 | 25.59 | C |
| ATOM | 1978 | NZ | LYS | A | 338 | 41.678 | −8.368 | 33.629 | 1.00 | 28.90 | N |
| ATOM | 1979 | N | PHE | A | 339 | 36.418 | −9.245 | 27.031 | 1.00 | 16.25 | N |
| ATOM | 1980 | CA | PHE | A | 339 | 36.290 | −9.013 | 25.601 | 1.00 | 16.45 | C |
| ATOM | 1981 | C | PHE | A | 339 | 35.290 | −9.970 | 24.963 | 1.00 | 19.22 | C |
| ATOM | 1982 | O | PHE | A | 339 | 35.060 | −11.071 | 25.469 | 1.00 | 19.37 | O |
| ATOM | 1983 | CB | PHE | A | 339 | 37.671 | −9.137 | 24.927 | 1.00 | 13.30 | C |
| ATOM | 1984 | CG | PHE | A | 339 | 38.255 | −10.532 | 24.960 | 1.00 | 15.28 | C |
| ATOM | 1985 | CD1 | PHE | A | 339 | 38.055 | −11.410 | 23.893 | 1.00 | 13.14 | C |
| ATOM | 1986 | CD2 | PHE | A | 339 | 39.009 | −10.963 | 26.049 | 1.00 | 15.20 | C |
| ATOM | 1987 | CE1 | PHE | A | 339 | 38.602 | −12.699 | 23.913 | 1.00 | 16.50 | C |
| ATOM | 1988 | CE2 | PHE | A | 339 | 39.561 | −12.252 | 26.082 | 1.00 | 15.77 | C |
| ATOM | 1989 | CZ | PHE | A | 339 | 39.357 | −13.120 | 25.011 | 1.00 | 14.31 | C |
| ATOM | 1990 | N | GLN | A | 340 | 34.664 | −9.528 | 23.877 | 1.00 | 17.10 | N |
| ATOM | 1991 | CA | GLN | A | 340 | 33.715 | −10.366 | 23.158 | 1.00 | 18.11 | C |
| ATOM | 1992 | C | GLN | A | 340 | 34.550 | −11.209 | 22.224 | 1.00 | 19.36 | C |
| ATOM | 1993 | O | GLN | A | 340 | 35.588 | −10.766 | 21.733 | 1.00 | 15.13 | O |
| ATOM | 1994 | CB | GLN | A | 340 | 32.730 | −9.537 | 22.324 | 1.00 | 18.68 | C |
| ATOM | 1995 | CG | GLN | A | 340 | 31.513 | −9.057 | 23.084 | 1.00 | 21.48 | C |
| ATOM | 1996 | CD | GLN | A | 340 | 31.873 | −8.127 | 24.213 | 1.00 | 22.35 | C |
| ATOM | 1997 | OE1 | GLN | A | 340 | 32.230 | −6.969 | 23.991 | 1.00 | 27.13 | O |
| ATOM | 1998 | NE2 | GLN | A | 340 | 31.794 | −8.633 | 25.438 | 1.00 | 21.82 | N |
| ATOM | 1999 | N | MET | A | 341 | 34.080 | −12.415 | 21.957 | 1.00 | 20.92 | N |
| ATOM | 2000 | CA | MET | A | 341 | 34.805 | −13.327 | 21.093 | 1.00 | 26.80 | C |
| ATOM | 2001 | C | MET | A | 341 | 33.826 | −14.022 | 20.165 | 1.00 | 25.81 | C |
| ATOM | 2002 | O | MET | A | 341 | 32.695 | −14.309 | 20.557 | 1.00 | 27.48 | O |
| ATOM | 2003 | CB | MET | A | 341 | 35.507 | −14.351 | 21.967 | 1.00 | 29.65 | C |
| ATOM | 2004 | CG | MET | A | 341 | 36.332 | −15.366 | 21.250 | 1.00 | 39.59 | C |
| ATOM | 2005 | SD | MET | A | 341 | 36.691 | −16.638 | 22.454 | 1.00 | 41.28 | S |
| ATOM | 2006 | CE | MET | A | 341 | 37.427 | −15.712 | 23.716 | 1.00 | 44.41 | C |
| ATOM | 2007 | N | GLN | A | 342 | 34.259 | −14.281 | 18.934 | 1.00 | 23.40 | N |
| ATOM | 2008 | CA | GLN | A | 342 | 33.418 | −14.958 | 17.959 | 1.00 | 21.29 | C |
| ATOM | 2009 | C | GLN | A | 342 | 34.284 | −15.869 | 17.115 | 1.00 | 23.26 | C |
| ATOM | 2010 | O | GLN | A | 342 | 35.423 | −15.529 | 16.775 | 1.00 | 20.86 | O |
| ATOM | 2011 | CB | GLN | A | 342 | 32.703 | −13.945 | 17.063 | 1.00 | 24.80 | C |
| ATOM | 2012 | CG | GLN | A | 342 | 31.724 | −13.057 | 17.811 | 1.00 | 32.33 | C |
| ATOM | 2013 | CD | GLN | A | 342 | 30.383 | −13.732 | 18.081 | 1.00 | 35.71 | C |
| ATOM | 2014 | OE1 | GLN | A | 342 | 30.290 | −14.959 | 18.154 | 1.00 | 37.16 | O |
| ATOM | 2015 | NE2 | GLN | A | 342 | 29.340 | −12.924 | 18.245 | 1.00 | 35.77 | N |
| ATOM | 2016 | N | VAL | A | 343 | 33.742 | −17.033 | 16.784 | 1.00 | 20.85 | N |
| ATOM | 2017 | CA | VAL | A | 343 | 34.464 | −18.001 | 15.979 | 1.00 | 19.35 | C |
| ATOM | 2018 | C | VAL | A | 343 | 33.875 | −18.004 | 14.573 | 1.00 | 22.70 | C |
| ATOM | 2019 | O | VAL | A | 343 | 32.668 | −18.179 | 14.401 | 1.00 | 23.73 | O |
| ATOM | 2020 | CB | VAL | A | 343 | 34.350 | −19.415 | 16.585 | 1.00 | 22.16 | C |
| ATOM | 2021 | CG1 | VAL | A | 343 | 35.095 | −20.423 | 15.707 | 1.00 | 21.87 | C |
| ATOM | 2022 | CG2 | VAL | A | 343 | 34.908 | −19.418 | 18.008 | 1.00 | 19.01 | C |
| ATOM | 2023 | N | LEU | A | 344 | 34.732 | −17.801 | 13.577 | 1.00 | 21.76 | N |
| ATOM | 2024 | CA | LEU | A | 344 | 34.321 | −17.787 | 12.174 | 1.00 | 26.16 | C |
| ATOM | 2025 | C | LEU | A | 344 | 34.903 | −19.016 | 11.465 | 1.00 | 28.38 | C |
| ATOM | 2026 | O | LEU | A | 344 | 35.744 | −19.715 | 12.027 | 1.00 | 25.64 | O |
| ATOM | 2027 | CB | LEU | A | 344 | 34.831 | −16.514 | 11.488 | 1.00 | 23.90 | C |
| ATOM | 2028 | CG | LEU | A | 344 | 34.505 | −15.166 | 12.138 | 1.00 | 27.47 | C |
| ATOM | 2029 | CD1 | LEU | A | 344 | 34.954 | −14.047 | 11.212 | 1.00 | 25.40 | C |
| ATOM | 2030 | CD2 | LEU | A | 344 | 33.021 | −15.059 | 12.406 | 1.00 | 28.30 | C |
| ATOM | 2031 | N | PRO | A | 345 | 34.462 | −19.295 | 10.221 | 1.00 | 32.79 | N |
| ATOM | 2032 | CA | PRO | A | 345 | 34.955 | −20.451 | 9.454 | 1.00 | 34.42 | C |
| ATOM | 2033 | C | PRO | A | 345 | 36.467 | −20.397 | 9.221 | 1.00 | 37.96 | C |
| ATOM | 2034 | O | PRO | A | 345 | 37.017 | −19.335 | 8.941 | 1.00 | 39.41 | O |
| ATOM | 2035 | CB | PRO | A | 345 | 34.161 | −20.363 | 8.154 | 1.00 | 35.54 | C |
| ATOM | 2036 | CG | PRO | A | 345 | 32.854 | −19.772 | 8.606 | 1.00 | 35.01 | C |
| ATOM | 2037 | CD | PRO | A | 345 | 33.321 | −18.663 | 9.532 | 1.00 | 33.83 | C |
| ATOM | 2038 | N | GLN | A | 346 | 37.133 | −21.544 | 9.317 | 1.00 | 39.60 | N |
| ATOM | 2039 | CA | GLN | A | 346 | 38.581 | −21.590 | 9.144 | 1.00 | 43.26 | C |
| ATOM | 2040 | C | GLN | A | 346 | 39.131 | −21.019 | 7.845 | 1.00 | 42.87 | C |
| ATOM | 2041 | O | GLN | A | 346 | 38.565 | −21.207 | 6.769 | 1.00 | 42.08 | O |
| ATOM | 2042 | CB | GLN | A | 346 | 39.109 | −23.017 | 9.320 | 1.00 | 48.04 | C |
| ATOM | 2043 | CG | GLN | A | 346 | 40.602 | −23.119 | 9.025 | 1.00 | 52.14 | C |
| ATOM | 2044 | CD | GLN | A | 346 | 41.312 | −24.151 | 9.871 | 1.00 | 58.42 | C |
| ATOM | 2045 | OE1 | GLN | A | 346 | 40.973 | −25.337 | 9.845 | 1.00 | 60.29 | O |
| ATOM | 2046 | NE2 | GLN | A | 346 | 42.313 | −23.705 | 10.631 | 1.00 | 57.79 | N |
| ATOM | 2047 | N | CYS | A | 347 | 40.255 | −20.320 | 7.978 | 1.00 | 44.17 | N |
| ATOM | 2048 | CA | CYS | A | 347 | 40.954 | −19.696 | 6.862 | 1.00 | 45.46 | C |
| ATOM | 2049 | C | CYS | A | 347 | 42.417 | −19.569 | 7.267 | 1.00 | 46.82 | C |
| ATOM | 2050 | O | CYS | A | 347 | 42.745 | −18.924 | 8.269 | 1.00 | 47.14 | O |
| ATOM | 2051 | CB | CYS | A | 347 | 40.380 | −18.307 | 6.568 | 1.00 | 44.34 | C |
| ATOM | 2052 | SG | CYS | A | 347 | 41.366 | −17.339 | 5.388 | 1.00 | 45.68 | S |
| ATOM | 2053 | N | GLY | A | 348 | 43.293 | −20.184 | 6.484 | 1.00 | 45.35 | N |
| ATOM | 2054 | CA | GLY | A | 348 | 44.705 | −20.135 | 6.798 | 1.00 | 44.28 | C |
| ATOM | 2055 | C | GLY | A | 348 | 45.325 | −18.773 | 6.570 | 1.00 | 43.69 | C |
| ATOM | 2056 | O | GLY | A | 348 | 46.360 | −18.453 | 7.158 | 1.00 | 43.84 | O |
| ATOM | 2057 | N | HIS | A | 349 | 44.700 | −17.968 | 5.718 | 1.00 | 41.71 | N |
| ATOM | 2058 | CA | HIS | A | 349 | 45.218 | −16.641 | 5.414 | 1.00 | 40.07 | C |
| ATOM | 2059 | C | HIS | A | 349 | 44.406 | −15.500 | 6.018 | 1.00 | 36.07 | C |
| ATOM | 2060 | O | HIS | A | 349 | 44.441 | −15.274 | 7.228 | 1.00 | 34.45 | O |
| ATOM | 2061 | CB | HIS | A | 349 | 45.324 | −16.458 | 3.897 | 1.00 | 42.20 | C |
| ATOM | 2062 | CG | HIS | A | 349 | 46.501 | −17.152 | 3.292 | 1.00 | 46.75 | C |
| ATOM | 2063 | ND1 | HIS | A | 349 | 46.660 | −18.521 | 3.331 | 1.00 | 49.92 | N |
| ATOM | 2064 | CD2 | HIS | A | 349 | 47.600 | −16.664 | 2.668 | 1.00 | 47.78 | C |
| ATOM | 2065 | CE1 | HIS | A | 349 | 47.807 | −18.845 | 2.760 | 1.00 | 50.36 | C |
| ATOM | 2066 | NE2 | HIS | A | 349 | 48.396 | −17.736 | 2.349 | 1.00 | 50.93 | N |
| ATOM | 2067 | N | ALA | A | 350 | 43.687 | −14.778 | 5.165 | 1.00 | 31.38 | N |
| ATOM | 2068 | CA | ALA | A | 350 | 42.877 | −13.655 | 5.611 | 1.00 | 27.15 | C |
| ATOM | 2069 | C | ALA | A | 350 | 41.386 | −13.949 | 5.520 | 1.00 | 22.92 | C |
| ATOM | 2070 | O | ALA | A | 350 | 40.810 | −13.959 | 4.430 | 1.00 | 21.23 | O |
| ATOM | 2071 | CB | ALA | A | 350 | 43.213 | −12.420 | 4.782 | 1.00 | 30.16 | C |
| ATOM | 2072 | N | VAL | A | 351 | 40.757 | −14.182 | 6.667 | 1.00 | 20.64 | N |
| ATOM | 2073 | CA | VAL | A | 351 | 39.327 | −14.456 | 6.681 | 1.00 | 17.72 | C |
| ATOM | 2074 | C | VAL | A | 351 | 38.521 | −13.250 | 6.173 | 1.00 | 16.70 | C |
| ATOM | 2075 | O | VAL | A | 351 | 37.420 | −13.418 | 5.643 | 1.00 | 14.51 | O |
| ATOM | 2076 | CB | VAL | A | 351 | 38.841 | −14.853 | 8.098 | 1.00 | 18.94 | C |
| ATOM | 2077 | CG1 | VAL | A | 351 | 38.979 | −13.672 | 9.047 | 1.00 | 18.05 | C |
| ATOM | 2078 | CG2 | VAL | A | 351 | 37.400 | −15.348 | 8.033 | 1.00 | 17.25 | C |
| ATOM | 2079 | N | HIS | A | 352 | 39.065 | −12.040 | 6.311 | 1.00 | 16.31 | N |
| ATOM | 2080 | CA | HIS | A | 352 | 38.343 | −10.861 | 5.830 | 1.00 | 15.74 | C |
| ATOM | 2081 | C | HIS | A | 352 | 38.298 | −10.822 | 4.300 | 1.00 | 18.89 | C |
| ATOM | 2082 | O | HIS | A | 352 | 37.460 | −10.129 | 3.715 | 1.00 | 17.72 | O |
| ATOM | 2083 | CB | HIS | A | 352 | 38.935 | −9.554 | 6.392 | 1.00 | 18.36 | C |
| ATOM | 2084 | CG | HIS | A | 352 | 40.422 | −9.439 | 6.266 | 1.00 | 21.51 | C |
| ATOM | 2085 | ND1 | HIS | A | 352 | 41.032 | −8.419 | 5.566 | 1.00 | 24.73 | N |
| ATOM | 2086 | CD2 | HIS | A | 352 | 41.422 | −10.172 | 6.807 | 1.00 | 22.93 | C |
| ATOM | 2087 | CE1 | HIS | A | 352 | 42.342 | −8.526 | 5.686 | 1.00 | 20.53 | C |
| ATOM | 2088 | NE2 | HIS | A | 352 | 42.606 | −9.581 | 6.434 | 1.00 | 26.94 | N |
| ATOM | 2089 | N | GLU | A | 353 | 39.199 | −11.560 | 3.653 | 1.00 | 15.96 | N |
| ATOM | 2090 | CA | GLU | A | 353 | 39.199 | −11.645 | 2.193 | 1.00 | 17.33 | C |
| ATOM | 2091 | C | GLU | A | 353 | 38.382 | −12.866 | 1.758 | 1.00 | 18.91 | C |
| ATOM | 2092 | O | GLU | A | 353 | 37.594 | −12.800 | 0.811 | 1.00 | 17.66 | O |
| ATOM | 2093 | CB | GLU | A | 353 | 40.622 | −11.783 | 1.629 | 1.00 | 18.66 | C |
| ATOM | 2094 | CG | GLU | A | 353 | 41.465 | −10.520 | 1.724 | 1.00 | 17.64 | C |
| ATOM | 2095 | CD | GLU | A | 353 | 42.518 | −10.429 | 0.633 | 1.00 | 22.35 | C |
| ATOM | 2096 | OE1 | GLU | A | 353 | 42.146 | −10.515 | −0.561 | 1.00 | 25.44 | O |
| ATOM | 2097 | OE2 | GLU | A | 353 | 43.711 | −10.259 | 0.959 | 1.00 | 19.40 | O |
| ATOM | 2098 | N | ASP | A | 354 | 38.568 | −13.980 | 2.459 | 1.00 | 18.27 | N |
| ATOM | 2099 | CA | ASP | A | 354 | 37.868 | −15.214 | 2.119 | 1.00 | 20.92 | C |
| ATOM | 2100 | C | ASP | A | 354 | 36.378 | −15.228 | 2.448 | 1.00 | 20.55 | C |
| ATOM | 2101 | O | ASP | A | 354 | 35.585 | −15.763 | 1.678 | 1.00 | 20.46 | O |
| ATOM | 2102 | CB | ASP | A | 354 | 38.551 | −16.407 | 2.795 | 1.00 | 26.52 | C |
| ATOM | 2103 | CG | ASP | A | 354 | 39.846 | −16.809 | 2.106 | 1.00 | 33.14 | C |
| ATOM | 2104 | OD1 | ASP | A | 354 | 40.307 | −16.076 | 1.202 | 1.00 | 34.79 | O |
| ATOM | 2105 | OD2 | ASP | A | 354 | 40.406 | −17.862 | 2.473 | 1.00 | 35.41 | O |
| ATOM | 2106 | N | ALA | A | 355 | 35.999 | −14.650 | 3.586 | 1.00 | 17.72 | N |
| ATOM | 2107 | CA | ALA | A | 355 | 34.595 | −14.617 | 4.008 | 1.00 | 17.44 | C |
| ATOM | 2108 | C | ALA | A | 355 | 34.264 | −13.215 | 4.520 | 1.00 | 18.23 | C |
| ATOM | 2109 | O | ALA | A | 355 | 33.933 | −13.029 | 5.689 | 1.00 | 18.14 | O |
| ATOM | 2110 | CB | ALA | A | 355 | 34.365 | −15.645 | 5.106 | 1.00 | 18.09 | C |
| ATOM | 2111 | N | PRO | A | 356 | 34.334 | −12.214 | 3.631 | 1.00 | 20.22 | N |
| ATOM | 2112 | CA | PRO | A | 356 | 34.066 | −10.808 | 3.942 | 1.00 | 18.17 | C |
| ATOM | 2113 | C | PRO | A | 356 | 32.777 | −10.541 | 4.711 | 1.00 | 21.39 | C |
| ATOM | 2114 | O | PRO | A | 356 | 32.777 | −9.773 | 5.673 | 1.00 | 17.59 | O |
| ATOM | 2115 | CB | PRO | A | 356 | 34.054 | −10.146 | 2.565 | 1.00 | 20.23 | C |
| ATOM | 2116 | CG | PRO | A | 356 | 34.928 | −11.043 | 1.736 | 1.00 | 24.36 | C |
| ATOM | 2117 | CD | PRO | A | 356 | 34.476 | −12.401 | 2.177 | 1.00 | 15.91 | C |
| ATOM | 2118 | N | ASP | A | 357 | 31.675 | −11.165 | 4.305 | 1.00 | 21.56 | N |
| ATOM | 2119 | CA | ASP | A | 357 | 30.422 | −10.897 | 4.998 | 1.00 | 23.78 | C |
| ATOM | 2120 | C | ASP | A | 357 | 30.313 | −11.538 | 6.367 | 1.00 | 21.79 | C |
| ATOM | 2121 | O | ASP | A | 357 | 29.552 | −11.068 | 7.214 | 1.00 | 21.71 | O |
| ATOM | 2122 | CB | ASP | A | 357 | 29.214 | −11.303 | 4.158 | 1.00 | 28.12 | C |
| ATOM | 2123 | CG | ASP | A | 357 | 27.932 | −10.680 | 4.678 | 1.00 | 35.82 | C |
| ATOM | 2124 | OD1 | ASP | A | 357 | 27.897 | −9.434 | 4.804 | 1.00 | 36.18 | O |
| ATOM | 2125 | OD2 | ASP | A | 357 | 26.969 | −11.420 | 4.972 | 1.00 | 38.58 | O |
| ATOM | 2126 | N | LYS | A | 358 | 31.053 | −12.617 | 6.594 | 1.00 | 20.71 | N |
| ATOM | 2127 | CA | LYS | A | 358 | 31.010 | −13.246 | 7.905 | 1.00 | 19.95 | C |
| ATOM | 2128 | C | LYS | A | 358 | 31.745 | −12.335 | 8.873 | 1.00 | 16.43 | C |
| ATOM | 2129 | O | LYS | A | 358 | 31.339 | −12.181 | 10.025 | 1.00 | 17.08 | O |
| ATOM | 2130 | CB | LYS | A | 358 | 31.653 | −14.630 | 7.873 | 1.00 | 21.89 | C |
| ATOM | 2131 | CG | LYS | A | 358 | 30.888 | −15.601 | 6.991 | 1.00 | 29.54 | C |
| ATOM | 2132 | CD | LYS | A | 358 | 31.184 | −17.044 | 7.346 | 1.00 | 33.06 | C |
| ATOM | 2133 | CE | LYS | A | 358 | 30.397 | −17.982 | 6.436 | 1.00 | 39.96 | C |
| ATOM | 2134 | NZ | LYS | A | 358 | 28.944 | −17.628 | 6.417 | 1.00 | 39.97 | N |
| ATOM | 2135 | N | VAL | A | 359 | 32.822 | −11.713 | 8.402 | 1.00 | 16.78 | N |
| ATOM | 2136 | CA | VAL | A | 359 | 33.566 | −10.801 | 9.259 | 1.00 | 14.92 | C |
| ATOM | 2137 | C | VAL | A | 359 | 32.695 | −9.561 | 9.488 | 1.00 | 15.40 | C |
| ATOM | 2138 | O | VAL | A | 359 | 32.570 | −9.084 | 10.615 | 1.00 | 15.39 | O |
| ATOM | 2139 | CB | VAL | A | 359 | 34.920 | −10.399 | 8.625 | 1.00 | 17.36 | C |
| ATOM | 2140 | CG1 | VAL | A | 359 | 35.640 | −9.384 | 9.512 | 1.00 | 14.71 | C |
| ATOM | 2141 | CG2 | VAL | A | 359 | 35.799 | −11.643 | 8.450 | 1.00 | 14.39 | C |
| ATOM | 2142 | N | ALA | A | 360 | 32.071 | −9.056 | 8.424 | 1.00 | 17.21 | N |
| ATOM | 2143 | CA | ALA | A | 360 | 31.214 | −7.873 | 8.543 | 1.00 | 15.07 | C |
| ATOM | 2144 | C | ALA | A | 360 | 30.056 | −8.138 | 9.506 | 1.00 | 15.89 | C |
| ATOM | 2145 | O | ALA | A | 360 | 29.705 | −7.285 | 10.318 | 1.00 | 18.38 | O |
| ATOM | 2146 | CB | ALA | A | 360 | 30.677 | −7.475 | 7.176 | 1.00 | 15.62 | C |
| ATOM | 2147 | N | GLU | A | 361 | 29.466 | −9.326 | 9.420 | 1.00 | 14.79 | N |
| ATOM | 2148 | CA | GLU | A | 361 | 28.359 | −9.680 | 10.301 | 1.00 | 21.38 | C |
| ATOM | 2149 | C | GLU | A | 361 | 28.816 | −9.619 | 11.765 | 1.00 | 17.02 | C |
| ATOM | 2150 | O | GLU | A | 361 | 28.148 | −9.027 | 12.614 | 1.00 | 16.93 | O |
| ATOM | 2151 | CB | GLU | A | 361 | 27.856 | −11.086 | 9.966 | 1.00 | 21.81 | C |
| ATOM | 2152 | CG | GLU | A | 361 | 26.553 | −11.462 | 10.635 | 1.00 | 37.83 | C |
| ATOM | 2153 | CD | GLU | A | 361 | 26.093 | −12.861 | 10.258 | 1.00 | 44.30 | C |
| ATOM | 2154 | OE1 | GLU | A | 361 | 25.981 | −13.150 | 9.047 | 1.00 | 51.87 | O |
| ATOM | 2155 | OE2 | GLU | A | 361 | 25.840 | −13.672 | 11.174 | 1.00 | 53.03 | O |
| ATOM | 2156 | N | ALA | A | 362 | 29.966 | −10.217 | 12.051 | 1.00 | 15.50 | N |
| ATOM | 2157 | CA | ALA | A | 362 | 30.499 | −10.221 | 13.410 | 1.00 | 16.58 | C |
| ATOM | 2158 | C | ALA | A | 362 | 30.800 | −8.799 | 13.897 | 1.00 | 16.84 | C |
| ATOM | 2159 | O | ALA | A | 362 | 30.479 | −8.441 | 15.031 | 1.00 | 14.42 | O |
| ATOM | 2160 | CB | ALA | A | 362 | 31.759 | −11.076 | 13.468 | 1.00 | 14.51 | C |
| ATOM | 2161 | N | VAL | A | 363 | 31.427 | −7.990 | 13.045 | 1.00 | 15.80 | N |
| ATOM | 2162 | CA | VAL | A | 363 | 31.743 | −6.614 | 13.417 | 1.00 | 14.42 | C |
| ATOM | 2163 | C | VAL | A | 363 | 30.462 | −5.807 | 13.635 | 1.00 | 16.07 | C |
| ATOM | 2164 | O | VAL | A | 363 | 30.342 | −5.073 | 14.613 | 1.00 | 15.60 | O |
| ATOM | 2165 | CB | VAL | A | 363 | 32.605 | −5.926 | 12.326 | 1.00 | 15.28 | C |
| ATOM | 2166 | CG1 | VAL | A | 363 | 32.746 | −4.426 | 12.626 | 1.00 | 14.04 | C |
| ATOM | 2167 | CG2 | VAL | A | 363 | 33.986 | −6.585 | 12.279 | 1.00 | 13.78 | C |
| ATOM | 2168 | N | ALA | A | 364 | 29.503 | −5.946 | 12.724 | 1.00 | 16.40 | N |
| ATOM | 2169 | CA | ALA | A | 364 | 28.232 | −5.226 | 12.841 | 1.00 | 16.36 | C |
| ATOM | 2170 | C | ALA | A | 364 | 27.520 | −5.554 | 14.163 | 1.00 | 16.75 | C |
| ATOM | 2171 | O | ALA | A | 364 | 27.024 | −4.664 | 14.855 | 1.00 | 15.27 | O |
| ATOM | 2172 | CB | ALA | A | 364 | 27.327 | −5.574 | 11.655 | 1.00 | 13.44 | C |
| ATOM | 2173 | N | THR | A | 365 | 27.473 | −6.836 | 14.506 | 1.00 | 15.53 | N |
| ATOM | 2174 | CA | THR | A | 365 | 26.828 | −7.274 | 15.741 | 1.00 | 18.96 | C |
| ATOM | 2175 | C | THR | A | 365 | 27.471 | −6.597 | 16.948 | 1.00 | 18.43 | C |
| ATOM | 2176 | O | THR | A | 365 | 26.778 | −6.112 | 17.845 | 1.00 | 15.61 | O |
| ATOM | 2177 | CB | THR | A | 365 | 26.931 | −8.808 | 15.888 | 1.00 | 22.23 | C |
| ATOM | 2178 | OG1 | THR | A | 365 | 26.225 | −9.426 | 14.807 | 1.00 | 21.57 | O |
| ATOM | 2179 | CG2 | THR | A | 365 | 26.330 | −9.275 | 17.209 | 1.00 | 25.12 | C |
| ATOM | 2180 | N | PHE | A | 366 | 28.800 | −6.547 | 16.946 | 1.00 | 15.50 | N |
| ATOM | 2181 | CA | PHE | A | 366 | 29.554 | −5.932 | 18.031 | 1.00 | 15.83 | C |
| ATOM | 2182 | C | PHE | A | 366 | 29.249 | −4.442 | 18.118 | 1.00 | 17.24 | C |
| ATOM | 2183 | O | PHE | A | 366 | 29.001 | −3.908 | 19.204 | 1.00 | 17.14 | O |
| ATOM | 2184 | CB | PHE | A | 366 | 31.050 | −6.131 | 17.799 | 1.00 | 15.12 | C |
| ATOM | 2185 | CG | PHE | A | 366 | 31.921 | −5.504 | 18.851 | 1.00 | 16.79 | C |
| ATOM | 2186 | CD1 | PHE | A | 366 | 32.148 | −6.150 | 20.063 | 1.00 | 19.28 | C |
| ATOM | 2187 | CD2 | PHE | A | 366 | 32.536 | −4.279 | 18.618 | 1.00 | 19.58 | C |
| ATOM | 2188 | CE1 | PHE | A | 366 | 32.984 | −5.585 | 21.024 | 1.00 | 21.07 | C |
| ATOM | 2189 | CE2 | PHE | A | 366 | 33.370 | −3.704 | 19.573 | 1.00 | 20.98 | C |
| ATOM | 2190 | CZ | PHE | A | 366 | 33.596 | −4.365 | 20.780 | 1.00 | 16.97 | C |
| ATOM | 2191 | N | LEU | A | 367 | 29.280 | −3.771 | 16.971 | 1.00 | 15.25 | N |
| ATOM | 2192 | CA | LEU | A | 367 | 29.009 | −2.338 | 16.918 | 1.00 | 16.95 | C |
| ATOM | 2193 | C | LEU | A | 367 | 27.619 | −2.012 | 17.456 | 1.00 | 17.32 | C |
| ATOM | 2194 | O | LEU | A | 367 | 27.438 | −1.063 | 18.223 | 1.00 | 20.03 | O |
| ATOM | 2195 | CB | LEU | A | 367 | 29.140 | −1.836 | 15.476 | 1.00 | 15.11 | C |
| ATOM | 2196 | CG | LEU | A | 367 | 30.563 | −1.819 | 14.903 | 1.00 | 18.28 | C |
| ATOM | 2197 | CD1 | LEU | A | 367 | 30.529 | −1.501 | 13.421 | 1.00 | 13.82 | C |
| ATOM | 2198 | CD2 | LEU | A | 367 | 31.395 | −0.797 | 15.650 | 1.00 | 17.44 | C |
| ATOM | 2199 | N | ILE | A | 368 | 26.639 | −2.802 | 17.044 | 1.00 | 16.24 | N |
| ATOM | 2200 | CA | ILE | A | 368 | 25.260 | −2.604 | 17.475 | 1.00 | 19.00 | C |
| ATOM | 2201 | C | ILE | A | 368 | 25.133 | −2.914 | 18.962 | 1.00 | 20.71 | C |
| ATOM | 2202 | O | ILE | A | 368 | 24.478 | −2.187 | 19.701 | 1.00 | 21.24 | O |
| ATOM | 2203 | CB | ILE | A | 368 | 24.311 | −3.507 | 16.657 | 1.00 | 19.65 | C |
| ATOM | 2204 | CG1 | ILE | A | 368 | 24.258 | −3.008 | 15.210 | 1.00 | 20.22 | C |
| ATOM | 2205 | CG2 | ILE | A | 368 | 22.918 | −3.528 | 17.278 | 1.00 | 24.26 | C |
| ATOM | 2206 | CD1 | ILE | A | 368 | 23.561 | −3.971 | 14.259 | 1.00 | 22.43 | C |
| ATOM | 2207 | N | ARG | A | 369 | 25.779 | −3.991 | 19.394 | 1.00 | 21.67 | N |
| ATOM | 2208 | CA | ARG | A | 369 | 25.751 | −4.405 | 20.792 | 1.00 | 20.36 | C |
| ATOM | 2209 | C | ARG | A | 369 | 26.163 | −3.280 | 21.735 | 1.00 | 21.79 | C |
| ATOM | 2210 | O | ARG | A | 369 | 25.547 | −3.077 | 22.783 | 1.00 | 19.47 | O |
| ATOM | 2211 | CB | ARG | A | 369 | 26.691 | −5.597 | 20.990 | 1.00 | 22.94 | C |
| ATOM | 2212 | CG | ARG | A | 369 | 26.880 | −6.016 | 22.433 | 1.00 | 24.44 | C |
| ATOM | 2213 | CD | ARG | A | 369 | 27.722 | −7.273 | 22.513 | 1.00 | 24.15 | C |
| ATOM | 2214 | NE | ARG | A | 369 | 27.794 | −7.791 | 23.873 | 1.00 | 27.88 | N |
| ATOM | 2215 | CZ | ARG | A | 369 | 27.820 | −9.085 | 24.174 | 1.00 | 24.66 | C |
| ATOM | 2216 | NH1 | ARG | A | 369 | 27.780 | −9.994 | 23.207 | 1.00 | 23.55 | N |
| ATOM | 2217 | NH2 | ARG | A | 369 | 27.872 | −9.467 | 25.443 | 1.00 | 26.92 | N |
| ATOM | 2218 | N | HIS | A | 370 | 27.211 | −2.553 | 21.359 | 1.00 | 19.99 | N |
| ATOM | 2219 | CA | HIS | A | 370 | 27.721 | −1.463 | 22.177 | 1.00 | 19.81 | C |
| ATOM | 2220 | C | HIS | A | 370 | 27.166 | −0.106 | 21.783 | 1.00 | 20.52 | C |
| ATOM | 2221 | O | HIS | A | 370 | 27.637 | 0.923 | 22.268 | 1.00 | 18.70 | O |
| ATOM | 2222 | CB | HIS | A | 370 | 29.247 | −1.444 | 22.102 | 1.00 | 21.88 | C |
| ATOM | 2223 | CG | HIS | A | 370 | 29.890 | −2.651 | 22.711 | 1.00 | 23.02 | C |
| ATOM | 2224 | ND1 | HIS | A | 370 | 29.956 | −2.851 | 24.073 | 1.00 | 24.03 | N |
| ATOM | 2225 | CD2 | HIS | A | 370 | 30.469 | −3.736 | 22.143 | 1.00 | 24.52 | C |
| ATOM | 2226 | CE1 | HIS | A | 370 | 30.549 | −4.006 | 24.320 | 1.00 | 22.41 | C |
| ATOM | 2227 | NE2 | HIS | A | 370 | 30.871 | −4.563 | 23.165 | 1.00 | 25.05 | N |
| ATOM | 2228 | N | ARG | A | 371 | 26.164 | −0.115 | 20.908 | 1.00 | 19.35 | N |
| ATOM | 2229 | CA | ARG | A | 371 | 25.519 | 1.106 | 20.430 | 1.00 | 21.85 | C |
| ATOM | 2230 | C | ARG | A | 371 | 26.474 | 2.081 | 19.744 | 1.00 | 24.32 | C |
| ATOM | 2231 | O | ARG | A | 371 | 26.278 | 3.299 | 19.799 | 1.00 | 21.41 | O |
| ATOM | 2232 | CB | ARG | A | 371 | 24.812 | 1.815 | 21.584 | 1.00 | 25.88 | C |
| ATOM | 2233 | CG | ARG | A | 371 | 23.734 | 0.977 | 22.245 | 1.00 | 36.27 | C |
| ATOM | 2234 | CD | ARG | A | 371 | 22.381 | 1.661 | 22.153 | 1.00 | 46.74 | C |
| ATOM | 2235 | NE | ARG | A | 371 | 21.355 | 0.958 | 22.921 | 1.00 | 55.32 | N |
| ATOM | 2236 | CZ | ARG | A | 371 | 20.094 | 1.367 | 23.036 | 1.00 | 58.22 | C |
| ATOM | 2237 | NH1 | ARG | A | 371 | 19.695 | 2.479 | 22.429 | 1.00 | 61.63 | N |
| ATOM | 2238 | NH2 | ARG | A | 371 | 19.231 | 0.667 | 23.760 | 1.00 | 59.47 | N |
| ATOM | 2239 | N | PHE | A | 372 | 27.505 | 1.549 | 19.097 | 1.00 | 20.06 | N |
| ATOM | 2240 | CA | PHE | A | 372 | 28.463 | 2.388 | 18.392 | 1.00 | 20.40 | C |
| ATOM | 2241 | C | PHE | A | 372 | 27.933 | 2.784 | 17.009 | 1.00 | 22.63 | C |
| ATOM | 2242 | O | PHE | A | 372 | 28.342 | 3.796 | 16.445 | 1.00 | 22.87 | O |
| ATOM | 2243 | CB | PHE | A | 372 | 29.803 | 1.663 | 18.231 | 1.00 | 19.05 | C |
| ATOM | 2244 | CG | PHE | A | 372 | 30.503 | 1.376 | 19.529 | 1.00 | 17.18 | C |
| ATOM | 2245 | CD1 | PHE | A | 372 | 30.351 | 2.223 | 20.623 | 1.00 | 20.51 | C |
| ATOM | 2246 | CD2 | PHE | A | 372 | 31.352 | 0.276 | 19.646 | 1.00 | 19.00 | C |
| ATOM | 2247 | CE1 | PHE | A | 372 | 31.034 | 1.982 | 21.815 | 1.00 | 19.00 | C |
| ATOM | 2248 | CE2 | PHE | A | 372 | 32.042 | 0.027 | 20.836 | 1.00 | 18.93 | C |
| ATOM | 2249 | CZ | PHE | A | 372 | 31.881 | 0.881 | 21.920 | 1.00 | 16.88 | C |
| ATOM | 2250 | N | ALA | A | 373 | 27.026 | 1.982 | 16.461 | 1.00 | 20.57 | N |
| ATOM | 2251 | CA | ALA | A | 373 | 26.472 | 2.282 | 15.147 | 1.00 | 23.56 | C |
| ATOM | 2252 | C | ALA | A | 373 | 25.037 | 1.797 | 15.016 | 1.00 | 25.88 | C |
| ATOM | 2253 | O | ALA | A | 373 | 24.598 | 0.895 | 15.736 | 1.00 | 24.59 | O |
| ATOM | 2254 | CB | ALA | A | 373 | 27.339 | 1.645 | 14.051 | 1.00 | 20.97 | C |
| ATOM | 2255 | N | GLU | A | 374 | 24.315 | 2.410 | 14.085 | 1.00 | 27.27 | N |
| ATOM | 2256 | CA | GLU | A | 374 | 22.929 | 2.056 | 13.819 | 1.00 | 29.68 | C |
| ATOM | 2257 | C | GLU | A | 374 | 22.856 | 1.067 | 12.668 | 1.00 | 29.33 | C |
| ATOM | 2258 | O | GLU | A | 374 | 23.588 | 1.187 | 11.680 | 1.00 | 26.91 | O |
| ATOM | 2259 | CB | GLU | A | 374 | 22.123 | 3.304 | 13.449 | 1.00 | 34.19 | C |
| ATOM | 2260 | CG | GLU | A | 374 | 21.962 | 4.295 | 14.581 | 1.00 | 42.62 | C |
| ATOM | 2261 | CD | GLU | A | 374 | 21.151 | 3.726 | 15.727 | 1.00 | 49.96 | C |
| ATOM | 2262 | OE1 | GLU | A | 374 | 19.983 | 3.342 | 15.496 | 1.00 | 51.95 | O |
| ATOM | 2263 | OE2 | GLU | A | 374 | 21.682 | 3.659 | 16.857 | 1.00 | 54.14 | O |
| ATOM | 2264 | N | PRO | A | 375 | 21.975 | 0.066 | 12.784 | 1.00 | 31.37 | N |
| ATOM | 2265 | CA | PRO | A | 375 | 21.820 | −0.934 | 11.727 | 1.00 | 34.49 | C |
| ATOM | 2266 | C | PRO | A | 375 | 21.262 | −0.197 | 10.517 | 1.00 | 35.45 | C |
| ATOM | 2267 | O | PRO | A | 375 | 20.566 | 0.806 | 10.678 | 1.00 | 33.40 | O |
| ATOM | 2268 | CB | PRO | A | 375 | 20.793 | −1.902 | 12.310 | 1.00 | 34.68 | C |
| ATOM | 2269 | CG | PRO | A | 375 | 20.952 | −1.729 | 13.797 | 1.00 | 37.34 | C |
| ATOM | 2270 | CD | PRO | A | 375 | 21.112 | −0.248 | 13.935 | 1.00 | 32.18 | C |
| ATOM | 2271 | N | ILE | A | 376 | 21.570 | −0.668 | 9.315 | 1.00 | 38.02 | N |
| ATOM | 2272 | CA | ILE | A | 376 | 21.049 | −0.017 | 8.121 | 1.00 | 41.91 | C |
| ATOM | 2273 | C | ILE | A | 376 | 19.559 | −0.323 | 8.032 | 1.00 | 44.15 | C |
| ATOM | 2274 | O | ILE | A | 376 | 18.757 | 0.635 | 8.077 | 1.00 | 48.91 | O |
| ATOM | 2275 | CB | ILE | A | 376 | 21.748 | −0.526 | 6.840 | 1.00 | 40.40 | C |
| ATOM | 2276 | CG1 | ILE | A | 376 | 23.170 | 0.033 | 6.764 | 1.00 | 38.84 | C |
| ATOM | 2277 | CG2 | ILE | A | 376 | 20.950 | −0.117 | 5.607 | 1.00 | 40.60 | C |
| ATOM | 2278 | CD1 | ILE | A | 376 | 23.235 | 1.539 | 6.628 | 1.00 | 35.64 | C |
| TER | 2279 | | ILE | A | 376 | | | | | | |
| HETATM | 2280 | O | HOH | A | 1001 | 44.541 | −1.741 | 5.991 | 1.00 | 13.87 | O |
| HETATM | 2281 | O | HOH | A | 1002 | 37.576 | −7.843 | 2.375 | 1.00 | 14.29 | O |
| HETATM | 2282 | O | HOH | A | 1003 | 35.976 | 7.666 | 5.977 | 1.00 | 14.14 | O |
| HETATM | 2283 | O | HOH | A | 1004 | 51.062 | −10.773 | 9.491 | 1.00 | 14.30 | O |
| HETATM | 2284 | O | HOH | A | 1005 | 33.754 | 3.036 | 23.823 | 1.00 | 14.81 | O |
| HETATM | 2285 | O | HOH | A | 1006 | 31.334 | −14.138 | 3.198 | 1.00 | 15.78 | O |
| HETATM | 2286 | O | HOH | A | 1007 | 52.845 | 1.865 | 9.629 | 1.00 | 16.04 | O |
| HETATM | 2287 | O | HOH | A | 1008 | 50.364 | −11.059 | 27.337 | 1.00 | 17.55 | O |
| HETATM | 2288 | O | HOH | A | 1009 | 51.721 | −4.417 | 0.145 | 1.00 | 16.50 | O |
| HETATM | 2289 | O | HOH | A | 1010 | 58.526 | −4.618 | 16.670 | 1.00 | 17.59 | O |
| HETATM | 2290 | O | HOH | A | 1011 | 45.279 | −12.417 | 31.358 | 1.00 | 16.83 | O |
| HETATM | 2291 | O | HOH | A | 1012 | 47.076 | −11.796 | 21.442 | 1.00 | 18.19 | O |
| HETATM | 2292 | O | HOH | A | 1013 | 45.310 | −8.615 | 5.731 | 1.00 | 18.23 | O |
| HETATM | 2293 | O | HOH | A | 1014 | 40.747 | −21.783 | 23.437 | 1.00 | 18.09 | O |
| HETATM | 2294 | O | HOH | A | 1015 | 53.404 | −7.961 | −0.631 | 1.00 | 18.80 | O |
| HETATM | 2295 | O | HOH | A | 1016 | 36.208 | −7.632 | −7.310 | 1.00 | 18.31 | O |
| HETATM | 2296 | O | HOH | A | 1017 | 43.233 | −0.329 | −7.087 | 1.00 | 18.43 | O |
| HETATM | 2297 | O | HOH | A | 1018 | 50.266 | −14.963 | 25.920 | 1.00 | 19.10 | O |
| HETATM | 2298 | O | HOH | A | 1019 | 38.994 | −7.238 | −0.711 | 1.00 | 18.72 | O |
| HETATM | 2299 | O | HOH | A | 1020 | 28.144 | 10.646 | 12.139 | 1.00 | 19.74 | O |
| HETATM | 2300 | O | HOH | A | 1021 | 42.425 | −14.977 | 8.932 | 1.00 | 20.12 | O |
| HETATM | 2301 | O | HOH | A | 1022 | 25.760 | 4.615 | 12.897 | 1.00 | 19.48 | O |
| HETATM | 2302 | O | HOH | A | 1023 | 44.227 | 5.097 | 20.971 | 1.00 | 20.08 | O |
| HETATM | 2303 | O | HOH | A | 1024 | 54.842 | −23.019 | 12.848 | 1.00 | 20.04 | O |
| HETATM | 2304 | O | HOH | A | 1025 | 49.170 | −20.068 | 26.341 | 1.00 | 19.76 | O |
| HETATM | 2305 | O | HOH | A | 1026 | 61.273 | −7.159 | 17.774 | 1.00 | 20.26 | O |
| HETATM | 2306 | O | HOH | A | 1027 | 30.437 | −10.226 | 17.205 | 1.00 | 20.31 | O |
| HETATM | 2307 | O | HOH | A | 1028 | 34.774 | −9.344 | −8.944 | 1.00 | 20.01 | O |
| HETATM | 2308 | O | HOH | A | 1029 | 46.125 | −6.599 | −0.359 | 1.00 | 20.19 | O |
| HETATM | 2309 | O | HOH | A | 1030 | 45.945 | −7.244 | 31.167 | 1.00 | 20.23 | O |
| HETATM | 2310 | O | HOH | A | 1031 | 29.446 | −9.296 | 19.916 | 1.00 | 20.15 | O |
| HETATM | 2311 | O | HOH | A | 1032 | 38.749 | −20.903 | 15.408 | 1.00 | 21.16 | O |
| HETATM | 2312 | O | HOH | A | 1033 | 34.419 | 8.749 | 3.663 | 1.00 | 20.74 | O |
| HETATM | 2313 | O | HOH | A | 1034 | 29.956 | 6.114 | 16.975 | 1.00 | 22.16 | O |
| HETATM | 2314 | O | HOH | A | 1035 | 28.078 | 16.399 | 10.570 | 1.00 | 22.41 | O |
| HETATM | 2315 | O | HOH | A | 1036 | 56.535 | 2.836 | 2.437 | 1.00 | 21.75 | O |
| HETATM | 2316 | O | HOH | A | 1037 | 30.475 | 15.021 | 11.532 | 1.00 | 22.10 | O |
| HETATM | 2317 | O | HOH | A | 1038 | 34.744 | 8.253 | 20.439 | 1.00 | 22.64 | O |
| HETATM | 2318 | O | HOH | A | 1039 | 47.236 | 0.340 | −2.032 | 1.00 | 20.97 | O |
| HETATM | 2319 | O | HOH | A | 1040 | 33.458 | −11.316 | 27.749 | 1.00 | 23.84 | O |
| HETATM | 2320 | O | HOH | A | 1041 | 41.473 | −21.839 | 26.020 | 1.00 | 22.41 | O |
| HETATM | 2321 | O | HOH | A | 1042 | 53.875 | −12.698 | 27.403 | 1.00 | 23.33 | O |
| HETATM | 2322 | O | HOH | A | 1043 | 34.558 | −15.100 | −13.059 | 1.00 | 23.48 | O |
| HETATM | 2323 | O | HOH | A | 1044 | 37.606 | 19.934 | 12.012 | 1.00 | 23.82 | O |
| HETATM | 2324 | O | HOH | A | 1045 | 42.593 | 13.666 | 14.596 | 1.00 | 22.17 | O |
| HETATM | 2325 | O | HOH | A | 1046 | 45.082 | 1.160 | −5.715 | 1.00 | 22.13 | O |
| HETATM | 2326 | O | HOH | A | 1047 | 40.988 | −22.791 | −7.380 | 1.00 | 23.59 | O |
| HETATM | 2327 | O | HOH | A | 1048 | 34.906 | 11.596 | 7.926 | 1.00 | 22.87 | O |
| HETATM | 2328 | O | HOH | A | 1049 | 42.741 | −2.828 | −7.003 | 1.00 | 23.75 | O |
| HETATM | 2329 | O | HOH | A | 1050 | 49.252 | −16.857 | 27.547 | 1.00 | 23.41 | O |
| HETATM | 2330 | O | HOH | A | 1051 | 53.722 | −21.209 | −5.993 | 1.00 | 23.64 | O |
| HETATM | 2331 | O | HOH | A | 1052 | 30.972 | −7.143 | −2.872 | 1.00 | 24.21 | O |
| HETATM | 2332 | O | HOH | A | 1053 | 41.475 | −23.168 | −2.860 | 1.00 | 24.50 | O |
| HETATM | 2333 | O | HOH | A | 1054 | 52.481 | −16.272 | 25.808 | 1.00 | 25.31 | O |
| HETATM | 2334 | O | HOH | A | 1055 | 27.787 | −1.931 | 0.170 | 1.00 | 25.09 | O |
| HETATM | 2335 | O | HOH | A | 1056 | 26.817 | 2.661 | 5.962 | 1.00 | 24.59 | O |
| HETATM | 2336 | O | HOH | A | 1057 | 55.599 | −18.626 | −19.131 | 1.00 | 24.44 | O |
| HETATM | 2337 | O | HOH | A | 1058 | 24.161 | −6.842 | 18.339 | 1.00 | 25.13 | O |
| HETATM | 2338 | O | HOH | A | 1059 | 54.117 | 3.972 | 19.284 | 1.00 | 25.24 | O |
| HETATM | 2339 | O | HOH | A | 1060 | 51.609 | −6.937 | 28.335 | 1.00 | 24.65 | O |
| HETATM | 2340 | O | HOH | A | 1061 | 35.481 | 20.999 | 10.602 | 1.00 | 23.40 | O |
| HETATM | 2341 | O | HOH | A | 1062 | 51.559 | −18.154 | −12.454 | 1.00 | 25.16 | O |
| HETATM | 2342 | O | HOH | A | 1063 | 29.216 | 8.000 | 14.994 | 1.00 | 23.95 | O |
| HETATM | 2343 | O | HOH | A | 1064 | 48.934 | −0.965 | −3.876 | 1.00 | 25.83 | O |
| HETATM | 2344 | O | HOH | A | 1065 | 37.083 | 9.732 | 7.247 | 1.00 | 25.14 | O |
| HETATM | 2345 | O | HOH | A | 1066 | 35.980 | 19.984 | 8.212 | 1.00 | 25.34 | O |
| HETATM | 2346 | O | HOH | A | 1067 | 49.417 | −20.391 | 23.484 | 1.00 | 24.63 | O |
| HETATM | 2347 | O | HOH | A | 1068 | 35.769 | 14.115 | 9.092 | 1.00 | 23.25 | O |
| HETATM | 2348 | O | HOH | A | 1069 | 41.352 | 10.188 | 17.580 | 1.00 | 24.66 | O |
| HETATM | 2349 | O | HOH | A | 1070 | 45.824 | 10.459 | 16.321 | 1.00 | 24.82 | O |
| HETATM | 2350 | O | HOH | A | 1071 | 34.765 | −12.229 | −9.327 | 1.00 | 24.91 | O |
| HETATM | 2351 | O | HOH | A | 1072 | 34.660 | 4.985 | −6.096 | 1.00 | 26.63 | O |
| HETATM | 2352 | O | HOH | A | 1073 | 59.024 | −16.702 | −10.505 | 1.00 | 26.35 | O |
| HETATM | 2353 | O | HOH | A | 1074 | 50.162 | 13.905 | 11.830 | 1.00 | 26.70 | O |
| HETATM | 2354 | O | HOH | A | 1075 | 55.148 | −13.548 | 23.624 | 1.00 | 25.84 | O |
| HETATM | 2355 | O | HOH | A | 1076 | 30.872 | −7.292 | −0.061 | 1.00 | 25.61 | O |
| HETATM | 2356 | O | HOH | A | 1077 | 39.209 | 9.703 | −4.397 | 1.00 | 27.34 | O |
| HETATM | 2357 | O | HOH | A | 1078 | 60.050 | 7.085 | 7.589 | 1.00 | 27.27 | O |
| HETATM | 2358 | O | HOH | A | 1079 | 43.085 | −14.897 | 1.653 | 1.00 | 26.55 | O |
| HETATM | 2359 | O | HOH | A | 1080 | 61.013 | −5.797 | 0.881 | 1.00 | 27.47 | O |
| HETATM | 2360 | O | HOH | A | 1081 | 47.054 | −17.938 | 11.616 | 1.00 | 27.76 | O |
| HETATM | 2361 | O | HOH | A | 1082 | 49.082 | −5.874 | −15.733 | 1.00 | 28.19 | O |
| HETATM | 2362 | O | HOH | A | 1083 | 38.050 | 17.216 | 11.489 | 1.00 | 25.26 | O |
| HETATM | 2363 | O | HOH | A | 1084 | 29.200 | 12.674 | 10.414 | 1.00 | 28.60 | O |
| HETATM | 2364 | O | HOH | A | 1085 | 46.556 | −7.404 | −16.231 | 1.00 | 27.79 | O |
| HETATM | 2365 | O | HOH | A | 1086 | 40.709 | −5.264 | −15.641 | 1.00 | 28.20 | O |
| HETATM | 2366 | O | HOH | A | 1087 | 37.322 | 6.184 | 26.419 | 1.00 | 26.56 | O |
| HETATM | 2367 | O | HOH | A | 1088 | 52.177 | −13.745 | 29.277 | 1.00 | 28.19 | O |
| HETATM | 2368 | O | HOH | A | 1089 | 55.569 | −24.475 | 10.728 | 1.00 | 29.37 | O |
| HETATM | 2369 | O | HOH | A | 1090 | 45.378 | 12.630 | −1.689 | 1.00 | 30.36 | O |
| HETATM | 2370 | O | HOH | A | 1091 | 46.171 | 0.327 | 28.307 | 1.00 | 28.63 | O |
| HETATM | 2371 | O | HOH | A | 1092 | 39.326 | 5.902 | 24.239 | 1.00 | 30.09 | O |
| HETATM | 2372 | O | HOH | A | 1093 | 32.223 | −13.542 | 27.838 | 1.00 | 28.90 | O |
| HETATM | 2373 | O | HOH | A | 1094 | 43.067 | 12.401 | 16.971 | 1.00 | 29.18 | O |
| HETATM | 2374 | O | HOH | A | 1095 | 27.581 | −15.749 | 17.561 | 1.00 | 29.08 | O |
| HETATM | 2375 | O | HOH | A | 1096 | 54.574 | −11.357 | 2.423 | 1.00 | 29.81 | O |
| HETATM | 2376 | O | HOH | A | 1097 | 48.294 | −16.939 | 16.510 | 1.00 | 28.90 | O |
| HETATM | 2377 | O | HOH | A | 1098 | 32.731 | −11.740 | −1.705 | 1.00 | 29.32 | O |
| HETATM | 2378 | O | HOH | A | 1099 | 41.122 | −21.176 | −4.757 | 1.00 | 29.77 | O |
| HETATM | 2379 | O | HOH | A | 1100 | 32.463 | 5.335 | 23.450 | 1.00 | 30.25 | O |
| HETATM | 2380 | O | HOH | A | 1101 | 61.621 | −10.743 | 16.507 | 1.00 | 29.85 | O |
| HETATM | 2381 | O | HOH | A | 1102 | 48.364 | 0.211 | −6.427 | 1.00 | 28.80 | O |
| HETATM | 2382 | O | HOH | A | 1103 | 31.164 | −2.243 | −3.765 | 1.00 | 29.92 | O |
| HETATM | 2383 | O | HOH | A | 1104 | 46.039 | −14.964 | 10.359 | 1.00 | 28.57 | O |
| HETATM | 2384 | O | HOH | A | 1105 | 54.614 | −22.599 | 4.369 | 1.00 | 30.52 | O |
| HETATM | 2385 | O | HOH | A | 1106 | 51.427 | −2.578 | −3.973 | 1.00 | 30.16 | O |
| HETATM | 2386 | O | HOH | A | 1107 | 55.486 | −5.977 | 22.457 | 1.00 | 30.60 | O |
| HETATM | 2387 | O | HOH | A | 1108 | 24.285 | −6.912 | 10.535 | 1.00 | 29.35 | O |
| HETATM | 2388 | O | HOH | A | 1109 | 29.729 | 10.950 | 14.394 | 1.00 | 30.56 | O |
| HETATM | 2389 | O | HOH | A | 1110 | 34.347 | −5.918 | 25.194 | 1.00 | 29.96 | O |
| HETATM | 2390 | O | HOH | A | 1111 | 31.894 | 2.204 | 25.659 | 1.00 | 30.27 | O |
| HETATM | 2391 | O | HOH | A | 1112 | 34.958 | 8.652 | 0.956 | 1.00 | 30.65 | O |
| HETATM | 2392 | O | HOH | A | 1113 | 29.880 | 6.919 | 19.377 | 1.00 | 31.76 | O |
| HETATM | 2393 | O | HOH | A | 1114 | 55.025 | 21.234 | 4.668 | 1.00 | 30.20 | O |
| HETATM | 2394 | O | HOH | A | 1115 | 52.803 | 22.142 | 5.543 | 1.00 | 30.14 | O |
| HETATM | 2395 | O | HOH | A | 1116 | 29.942 | −14.072 | 14.306 | 1.00 | 32.19 | O |
| HETATM | 2396 | O | HOH | A | 1117 | 22.574 | −5.092 | 10.073 | 1.00 | 30.32 | O |
| HETATM | 2397 | O | HOH | A | 1118 | 29.916 | −4.785 | −3.859 | 1.00 | 31.80 | O |
| HETATM | 2398 | O | HOH | A | 1119 | 29.548 | −0.956 | 25.863 | 1.00 | 31.91 | O |
| HETATM | 2399 | O | HOH | A | 1120 | 41.592 | −19.855 | 32.942 | 1.00 | 31.69 | O |
| HETATM | 2400 | O | HOH | A | 1121 | 58.069 | −2.532 | 0.564 | 1.00 | 32.33 | O |
| HETATM | 2401 | O | HOH | A | 1122 | 54.364 | 8.798 | 10.755 | 1.00 | 32.00 | O |
| HETATM | 2402 | O | HOH | A | 1123 | 49.852 | −5.250 | 29.827 | 1.00 | 32.22 | O |
| HETATM | 2403 | O | HOH | A | 1124 | 37.887 | 11.806 | 5.646 | 1.00 | 31.25 | O |
| HETATM | 2404 | O | HOH | A | 1125 | 52.918 | −17.804 | −19.315 | 1.00 | 32.15 | O |
| HETATM | 2405 | O | HOH | A | 1126 | 40.418 | 5.651 | 29.553 | 1.00 | 34.59 | O |
| HETATM | 2406 | O | HOH | A | 1127 | 39.712 | −20.914 | −14.236 | 1.00 | 33.60 | O |
| HETATM | 2407 | O | HOH | A | 1128 | 51.038 | 7.398 | −0.479 | 1.00 | 32.89 | O |
| HETATM | 2408 | O | HOH | A | 1129 | 44.435 | −19.843 | 31.006 | 1.00 | 32.37 | O |
| HETATM | 2409 | O | HOH | A | 1130 | 53.551 | −21.401 | 20.468 | 1.00 | 33.04 | O |
| HETATM | 2410 | O | HOH | A | 1131 | 36.170 | −17.967 | −5.653 | 1.00 | 32.84 | O |
| HETATM | 2411 | O | HOH | A | 1132 | 58.857 | −15.142 | −4.189 | 1.00 | 33.19 | O |
| HETATM | 2412 | O | HOH | A | 1133 | 57.019 | −12.167 | 1.918 | 1.00 | 33.20 | O |
| HETATM | 2413 | O | HOH | A | 1134 | 28.339 | −6.197 | 0.046 | 1.00 | 33.81 | O |
| HETATM | 2414 | O | HOH | A | 1135 | 30.028 | −11.824 | 21.076 | 1.00 | 37.06 | O |
| HETATM | 2415 | O | HOH | A | 1136 | 46.649 | −12.942 | 0.596 | 1.00 | 33.53 | O |
| HETATM | 2416 | O | HOH | A | 1137 | 44.217 | −22.138 | 29.535 | 1.00 | 33.83 | O |
| HETATM | 2417 | O | HOH | A | 1138 | 41.995 | 12.133 | 3.522 | 1.00 | 33.78 | O |
| HETATM | 2418 | O | HOH | A | 1139 | 27.837 | 6.613 | 5.885 | 1.00 | 32.04 | O |
| HETATM | 2419 | O | HOH | A | 1140 | 44.003 | −22.828 | 25.162 | 1.00 | 34.66 | O |
| HETATM | 2420 | O | HOH | A | 1141 | 35.280 | −12.767 | −11.775 | 1.00 | 33.94 | O |
| HETATM | 2421 | O | HOH | A | 1142 | 59.294 | −23.019 | 0.186 | 1.00 | 32.22 | O |
| HETATM | 2422 | O | HOH | A | 1143 | 60.776 | −3.895 | 18.080 | 1.00 | 33.26 | O |
| HETATM | 2423 | O | HOH | A | 1144 | 46.622 | −13.206 | 33.541 | 1.00 | 32.59 | O |
| HETATM | 2424 | O | HOH | A | 1145 | 54.578 | −10.146 | 0.187 | 1.00 | 33.18 | O |
| HETATM | 2425 | O | HOH | A | 1146 | 30.179 | −16.798 | 14.174 | 1.00 | 34.45 | O |
| HETATM | 2426 | O | HOH | A | 1147 | 48.951 | 10.493 | 18.143 | 1.00 | 37.02 | O |
| HETATM | 2427 | O | HOH | A | 1148 | 34.675 | −19.080 | 4.910 | 1.00 | 34.91 | O |
| HETATM | 2428 | O | HOH | A | 1149 | 41.007 | −13.586 | −16.807 | 1.00 | 32.42 | O |
| HETATM | 2429 | O | HOH | A | 1150 | 35.482 | 1.947 | −10.533 | 1.00 | 32.61 | O |
| HETATM | 2430 | O | HOH | A | 1151 | 33.403 | −16.975 | −11.463 | 1.00 | 36.55 | O |
| HETATM | 2431 | O | HOH | A | 1152 | 48.642 | −14.430 | 11.474 | 1.00 | 35.25 | O |
| HETATM | 2432 | O | HOH | A | 1153 | 57.078 | −14.500 | −1.895 | 1.00 | 35.57 | O |
| HETATM | 2433 | O | HOH | A | 1154 | 34.340 | −18.057 | −9.119 | 1.00 | 36.86 | O |
| HETATM | 2434 | O | HOH | A | 1155 | 46.005 | 3.947 | −6.939 | 1.00 | 36.16 | O |
| HETATM | 2435 | O | HOH | A | 1156 | 26.522 | −9.854 | 20.578 | 1.00 | 35.66 | O |
| HETATM | 2436 | O | HOH | A | 1157 | 22.712 | −7.200 | 15.928 | 1.00 | 37.89 | O |
| HETATM | 2437 | O | HOH | A | 1158 | 28.322 | −6.358 | 26.675 | 1.00 | 34.65 | O |
| HETATM | 2438 | O | HOH | A | 1159 | 52.372 | −23.753 | 18.446 | 1.00 | 37.81 | O |
| HETATM | 2439 | O | HOH | A | 1160 | 53.055 | 2.091 | 20.869 | 1.00 | 35.79 | O |
| HETATM | 2440 | O | HOH | A | 1161 | 42.807 | −18.801 | 2.083 | 1.00 | 36.11 | O |
| HETATM | 2441 | O | HOH | A | 1162 | 45.986 | 18.235 | 11.643 | 1.00 | 39.40 | O |
| HETATM | 2442 | O | HOH | A | 1163 | 27.245 | −17.614 | 15.485 | 1.00 | 37.74 | O |
| HETATM | 2443 | O | HOH | A | 1164 | 35.909 | 7.297 | 23.142 | 1.00 | 36.32 | O |
| HETATM | 2444 | O | HOH | A | 1165 | 53.503 | −0.696 | −8.992 | 1.00 | 37.19 | O |
| HETATM | 2445 | O | HOH | A | 1166 | 51.534 | 0.417 | 24.687 | 1.00 | 38.41 | O |
| HETATM | 2446 | O | HOH | A | 1167 | 43.663 | −10.614 | 32.824 | 1.00 | 35.80 | O |
| HETATM | 2447 | O | HOH | A | 1168 | 36.828 | 9.596 | −5.262 | 1.00 | 38.46 | O |
| HETATM | 2448 | O | HOH | A | 1169 | 64.522 | −9.159 | 9.860 | 1.00 | 37.21 | O |
| HETATM | 2449 | O | HOH | A | 1170 | 53.274 | −0.971 | 22.986 | 1.00 | 37.86 | O |
| HETATM | 2450 | O | HOH | A | 1171 | 38.461 | −20.319 | −5.116 | 1.00 | 37.98 | O |
| HETATM | 2451 | O | HOH | A | 1172 | 51.041 | 8.850 | 16.944 | 1.00 | 27.43 | O |
| HETATM | 2452 | O | HOH | A | 1173 | 24.507 | 0.881 | 2.703 | 1.00 | 42.32 | O |
| HETATM | 2453 | O | HOH | A | 1174 | 38.911 | 4.558 | 27.550 | 1.00 | 38.50 | O |
| HETATM | 2454 | O | HOH | A | 1175 | 54.570 | 1.678 | −1.458 | 1.00 | 37.53 | O |
| HETATM | 2455 | O | HOH | A | 1176 | 43.001 | −2.143 | −11.305 | 1.00 | 36.85 | O |
| HETATM | 2456 | O | HOH | A | 1177 | 38.680 | 11.712 | 0.464 | 1.00 | 39.52 | O |
| HETATM | 2457 | O | HOH | A | 1178 | 43.438 | −6.387 | 32.081 | 1.00 | 39.16 | O |
| HETATM | 2458 | O | HOH | A | 1179 | 47.740 | −13.756 | 5.634 | 1.00 | 37.32 | O |
| HETATM | 2459 | O | HOH | A | 1180 | 37.266 | −18.529 | 5.630 | 1.00 | 40.74 | O |
| HETATM | 2460 | O | HOH | A | 1181 | 43.714 | 0.064 | −9.657 | 1.00 | 34.99 | O |
| HETATM | 2461 | O | HOH | A | 1182 | 47.867 | 15.109 | 3.623 | 1.00 | 38.33 | O |
| HETATM | 2462 | O | HOH | A | 1183 | 54.026 | −25.135 | 6.962 | 1.00 | 37.18 | O |
| HETATM | 2463 | O | HOH | A | 1184 | 45.352 | 12.922 | 17.738 | 1.00 | 38.00 | O |
| HETATM | 2464 | O | HOH | A | 1185 | 53.020 | −12.620 | 31.537 | 1.00 | 39.68 | O |
| HETATM | 2465 | O | HOH | A | 1186 | 50.273 | −15.534 | 9.455 | 1.00 | 38.16 | O |
| HETATM | 2466 | O | HOH | A | 1187 | 36.835 | 14.488 | 18.482 | 1.00 | 38.04 | O |
| HETATM | 2467 | O | HOH | A | 1188 | 48.759 | 1.562 | 28.474 | 1.00 | 37.95 | O |
| HETATM | 2468 | O | HOH | A | 1189 | 38.824 | −25.032 | −6.083 | 1.00 | 40.21 | O |
| HETATM | 2469 | O | HOH | A | 1190 | 41.510 | 9.928 | −6.580 | 1.00 | 39.05 | O |
| HETATM | 2470 | O | HOH | A | 1191 | 59.779 | 7.252 | 10.852 | 1.00 | 35.92 | O |
| HETATM | 2471 | O | HOH | A | 1192 | 52.983 | −26.050 | 9.414 | 1.00 | 39.51 | O |
| HETATM | 2472 | O | HOH | A | 1193 | 59.830 | 10.924 | 6.896 | 1.00 | 39.07 | O |
| HETATM | 2473 | O | HOH | A | 1194 | 38.171 | −6.454 | −16.474 | 1.00 | 37.38 | O |
| HETATM | 2474 | O | HOH | A | 1195 | 39.019 | −20.677 | 31.228 | 1.00 | 50.07 | O |
| HETATM | 2475 | O | HOH | A | 1196 | 51.674 | −10.939 | −0.082 | 1.00 | 39.34 | O |
| HETATM | 2476 | O | HOH | A | 1197 | 36.701 | −19.109 | −8.204 | 1.00 | 39.64 | O |
| HETATM | 2477 | O | HOH | A | 1198 | 57.090 | −12.873 | −14.644 | 1.00 | 40.99 | O |
| HETATM | 2478 | O | HOH | A | 1199 | 43.078 | 16.586 | 6.010 | 1.00 | 39.69 | O |
| HETATM | 2479 | O | HOH | A | 1200 | 47.746 | −15.154 | 8.025 | 1.00 | 41.92 | O |
| HETATM | 2480 | O | HOH | A | 1201 | 26.653 | −7.334 | 4.081 | 1.00 | 40.68 | O |
| HETATM | 2481 | O | HOH | A | 1202 | 39.832 | 13.762 | 6.620 | 1.00 | 42.93 | O |
| HETATM | 2482 | O | HOH | A | 1203 | 37.829 | 17.751 | 8.751 | 1.00 | 40.09 | O |
| HETATM | 2483 | O | HOH | A | 1204 | 46.430 | −13.431 | −17.852 | 1.00 | 41.24 | O |
| HETATM | 2484 | O | HOH | A | 1205 | 37.769 | −5.027 | −13.905 | 1.00 | 39.63 | O |
| HETATM | 2485 | O | HOH | A | 1206 | 27.311 | 14.099 | 9.140 | 1.00 | 41.12 | O |
| HETATM | 2486 | O | HOH | A | 1207 | 60.558 | −16.510 | −2.716 | 1.00 | 38.38 | O |
| HETATM | 2487 | O | HOH | A | 1208 | 32.676 | −5.039 | −8.833 | 1.00 | 38.34 | O |
| HETATM | 2488 | O | HOH | A | 1209 | 58.692 | 2.408 | 0.620 | 1.00 | 42.64 | O |
| HETATM | 2489 | O | HOH | A | 1210 | 41.633 | 4.653 | 24.653 | 1.00 | 40.23 | O |
| HETATM | 2490 | O | HOH | A | 1211 | 33.704 | −3.752 | 27.614 | 1.00 | 44.14 | O |
| HETATM | 2491 | O | HOH | A | 1212 | 52.311 | −12.384 | 3.646 | 1.00 | 41.14 | O |
| HETATM | 2492 | O | HOH | A | 1213 | 56.505 | 0.810 | −7.094 | 1.00 | 40.74 | O |
| HETATM | 2493 | O | HOH | A | 1214 | 30.319 | −8.010 | −6.923 | 1.00 | 42.51 | O |
| HETATM | 2494 | O | HOH | A | 1215 | 60.951 | 15.757 | 4.419 | 1.00 | 45.06 | O |
| HETATM | 2495 | O | HOH | A | 1216 | 39.280 | 12.173 | −3.221 | 1.00 | 39.14 | O |
| HETATM | 2496 | O | HOH | A | 1217 | 28.412 | 1.193 | 24.927 | 1.00 | 43.79 | O |
| HETATM | 2497 | O | HOH | A | 1218 | 55.347 | −14.928 | 7.553 | 1.00 | 40.30 | O |
| HETATM | 2498 | O | HOH | A | 1219 | 47.711 | −16.133 | 13.435 | 1.00 | 42.38 | O |
| HETATM | 2499 | O | HOH | A | 1220 | 43.424 | 10.984 | −4.411 | 1.00 | 41.34 | O |
| HETATM | 2500 | O | HOH | A | 1221 | 55.905 | −20.770 | −6.947 | 1.00 | 44.28 | O |
| HETATM | 2501 | O | HOH | A | 1222 | 44.859 | 14.526 | 5.022 | 1.00 | 40.07 | O |
| HETATM | 2502 | O | HOH | A | 1223 | 32.080 | −9.126 | −8.490 | 1.00 | 38.64 | O |
| HETATM | 2503 | O | HOH | A | 1224 | 59.488 | −24.680 | −2.609 | 1.00 | 41.42 | O |
| HETATM | 2504 | O | HOH | A | 1225 | 41.234 | 8.052 | 24.281 | 1.00 | 43.47 | O |
| HETATM | 2505 | O | HOH | A | 1226 | 25.203 | −0.605 | 0.374 | 1.00 | 41.69 | O |
| HETATM | 2506 | O | HOH | A | 1227 | 29.025 | 4.232 | 1.962 | 1.00 | 44.44 | O |
| HETATM | 2507 | O | HOH | A | 1228 | 33.287 | 1.021 | 28.150 | 1.00 | 42.91 | O |
| HETATM | 2508 | O | HOH | A | 1229 | 27.811 | 3.537 | 22.897 | 1.00 | 39.30 | O |
| HETATM | 2509 | O | HOH | A | 1230 | 32.801 | 8.437 | −4.899 | 1.00 | 44.80 | O |
| HETATM | 2510 | O | HOH | A | 1231 | 59.585 | −11.007 | −2.044 | 1.00 | 43.27 | O |
| HETATM | 2511 | O | HOH | A | 1232 | 37.638 | −21.350 | 12.927 | 1.00 | 42.40 | O |
| HETATM | 2512 | O | HOH | A | 1233 | 64.218 | −9.680 | 18.034 | 1.00 | 39.48 | O |
| HETATM | 2513 | O | HOH | A | 1234 | 21.821 | −2.809 | 2.930 | 1.00 | 47.88 | O |
| HETATM | 2514 | O | HOH | A | 1235 | 47.066 | −15.436 | −15.988 | 1.00 | 40.89 | O |
| HETATM | 2515 | O | HOH | A | 1236 | 50.752 | −17.525 | 30.000 | 1.00 | 42.17 | O |
| HETATM | 2516 | O | HOH | A | 1237 | 49.174 | −2.273 | 29.611 | 1.00 | 42.05 | O |
| HETATM | 2517 | O | HOH | A | 1238 | 58.040 | −0.696 | −3.120 | 1.00 | 42.03 | O |
| HETATM | 2518 | O | HOH | A | 1239 | 49.097 | 16.356 | 11.805 | 1.00 | 44.47 | O |
| HETATM | 2519 | O | HOH | A | 1240 | 25.644 | 12.594 | 5.879 | 1.00 | 44.05 | O |
| HETATM | 2520 | O | HOH | A | 1241 | 61.658 | 5.328 | 11.449 | 1.00 | 45.31 | O |
| HETATM | 2521 | O | HOH | A | 1242 | 42.838 | 2.231 | 24.564 | 1.00 | 44.41 | O |
| HETATM | 2522 | O | HOH | A | 1243 | 25.331 | 10.229 | 12.622 | 1.00 | 44.84 | O |
| HETATM | 2523 | O | HOH | A | 1244 | 61.029 | −0.252 | 19.183 | 1.00 | 39.90 | O |
| HETATM | 2524 | O | HOH | A | 1245 | 37.007 | −19.556 | −18.290 | 1.00 | 45.46 | O |
| HETATM | 2525 | O | HOH | A | 1246 | 48.865 | −17.604 | 9.407 | 1.00 | 40.51 | O |
| HETATM | 2526 | O | HOH | A | 1247 | 46.250 | 14.588 | 1.665 | 1.00 | 43.18 | O |
| HETATM | 2527 | O | HOH | A | 1248 | 61.943 | −4.351 | 7.724 | 1.00 | 47.20 | O |
| HETATM | 2528 | O | HOH | A | 1249 | 35.349 | −7.803 | 33.043 | 1.00 | 45.31 | O |
| HETATM | 2529 | O | HOH | A | 1250 | 42.982 | −5.401 | −16.890 | 1.00 | 45.10 | O |
| HETATM | 2530 | O | HOH | A | 1251 | 44.528 | −17.436 | 9.933 | 1.00 | 46.75 | O |
| HETATM | 2531 | O | HOH | A | 1252 | 51.484 | −0.639 | 28.882 | 1.00 | 42.66 | O |
| HETATM | 2532 | O | HOH | A | 1253 | 59.386 | 3.569 | 20.419 | 1.00 | 43.40 | O |
| HETATM | 2533 | O | HOH | A | 1254 | 45.333 | 2.989 | 26.845 | 1.00 | 42.73 | O |
| HETATM | 2534 | O | HOH | A | 1255 | 51.911 | 15.759 | 9.929 | 1.00 | 47.16 | O |
| HETATM | 2535 | O | HOH | A | 1256 | 44.242 | −7.789 | −17.431 | 1.00 | 45.24 | O |
| HETATM | 2536 | O | HOH | A | 1257 | 38.735 | −23.681 | 1.597 | 1.00 | 47.56 | O |
| HETATM | 2537 | O | HOH | A | 1258 | 43.742 | 13.833 | 2.844 | 1.00 | 46.73 | O |
| HETATM | 2538 | O | HOH | A | 1259 | 51.678 | −21.410 | 22.939 | 1.00 | 43.27 | O |
| HETATM | 2539 | O | HOH | A | 1260 | 22.780 | −6.487 | 21.137 | 1.00 | 45.22 | O |
| HETATM | 2540 | O | HOH | A | 1261 | 50.747 | −21.142 | 7.218 | 1.00 | 43.72 | O |
| HETATM | 2541 | O | HOH | A | 1262 | 54.828 | 0.229 | 21.229 | 1.00 | 44.68 | O |
| HETATM | 2542 | O | HOH | A | 1263 | 55.679 | −8.986 | −18.553 | 1.00 | 49.30 | O |
| HETATM | 2543 | O | HOH | A | 1264 | 48.357 | 5.270 | −7.160 | 1.00 | 48.06 | O |
| HETATM | 2544 | O | HOH | A | 1265 | 24.231 | −8.724 | 20.017 | 1.00 | 43.97 | O |
| HETATM | 2545 | O | HOH | A | 1266 | 47.047 | −20.275 | 32.787 | 1.00 | 52.43 | O |
| HETATM | 2546 | O | HOH | A | 1267 | 32.721 | 9.476 | 0.051 | 1.00 | 41.39 | O |
| HETATM | 2547 | O | HOH | A | 1268 | 40.535 | −14.221 | 37.295 | 1.00 | 50.37 | O |
| HETATM | 2548 | O | HOH | A | 1269 | 54.580 | −18.853 | 23.017 | 1.00 | 49.05 | O |
| HETATM | 2549 | O | HOH | A | 1270 | 58.779 | −13.577 | 3.446 | 1.00 | 49.31 | O |
| HETATM | 2550 | O | HOH | A | 1271 | 45.356 | −21.596 | 27.181 | 1.00 | 49.84 | O |
| HETATM | 2551 | O | HOH | A | 1272 | 29.544 | −9.942 | −3.525 | 1.00 | 48.31 | O |
| HETATM | 2552 | O | HOH | A | 1273 | 53.746 | −5.696 | 27.683 | 1.00 | 45.67 | O |
| HETATM | 2553 | O | HOH | A | 1274 | 50.224 | −2.474 | −7.518 | 1.00 | 44.90 | O |
| HETATM | 2554 | O | HOH | A | 1275 | 43.801 | 4.922 | −8.648 | 1.00 | 48.90 | O |
| HETATM | 2555 | O | HOH | A | 1276 | 29.916 | −1.024 | −11.577 | 1.00 | 49.98 | O |
| HETATM | 2556 | O | HOH | A | 1277 | 61.805 | −4.291 | −14.915 | 1.00 | 53.96 | O |
| HETATM | 2557 | O | HOH | A | 1278 | 52.005 | −9.886 | 29.693 | 1.00 | 51.43 | O |
| HETATM | 2558 | O | HOH | A | 1279 | 33.308 | −11.128 | −13.204 | 1.00 | 53.16 | O |
| HETATM | 2559 | O | HOH | A | 1280 | 48.476 | −7.454 | 32.387 | 1.00 | 50.48 | O |
| HETATM | 2560 | O | HOH | A | 1281 | 52.397 | 6.035 | −4.067 | 1.00 | 49.10 | O |
| HETATM | 2561 | O | HOH | A | 1282 | 23.267 | 0.955 | 17.978 | 1.00 | 51.21 | O |
| HETATM | 2562 | O | HOH | A | 1283 | 26.031 | 6.313 | 18.268 | 1.00 | 51.67 | O |

| | | | | | | | | | | |
|---|---|---|---|---|---|---|---|---|---|---|
| MASTER | 286 0 | 0 | 14 | 10 | 0 | 0 | 6 2561 | 1 | 0 | 25 |
| END | | | | | | | | | | |

APPENDIX B
CRYSTAL COORDINATES OF COMPLEX BETWEEN PP2A-SPECIFIC METHYLESTERASE PME-1 AND
PP2A

| | |
|---|---|
| HEADER | HYDROLASE 01-FEB-08 3C5W |
| TITLE | COMPLEX BETWEEN PP2A-SPECIFIC METHYLESTERASE PME-1 AND PP2A |

| | | |
|---|---|---|
| TITLE | 2 | CORE ENZYME |

| | |
|---|---|
| COMPND | MOL_ID: 1; |

| | | |
|---|---|---|
| COMPND | 2 | MOLECULE: PP2A A SUBUNIT; |
| COMPND | 3 | CHAIN: A; |
| COMPND | 4 | ENGINEERED: YES; |
| COMPND | 5 | MOL_ID: 2; |
| COMPND | 6 | MOLECULE: PP2A C SUBUNIT; |
| COMPND | 7 | CHAIN: C; |
| COMPND | 8 | ENGINEERED: YES; |
| COMPND | 9 | MOL_ID: 3; |
| COMPND | 10 | MOLECULE: PP2A-SPECIFIC METHYLESTERASE PME-1; |
| COMPND | 11 | CHAIN: P; |
| COMPND | 12 | ENGINEERED: YES |

| | |
|---|---|
| SOURCE | MOL_ID: 1; |

| | | |
|---|---|---|
| SOURCE | 2 | ORGANISM_SCIENTIFIC: HOMO SAPIENS; |
| SOURCE | 3 | ORGANISM_COMMON: HUMAN; |
| SOURCE | 4 | GENUS: HOMO; |
| SOURCE | 5 | EXPRESSION_SYSTEM: ESCHERICHIA COLI; |
| SOURCE | 6 | EXPRESSION_SYSTEM_GENUS: ESCHERICHIA; |
| SOURCE | 7 | MOL_ID: 2; |
| SOURCE | 8 | ORGANISM_SCIENTIFIC: HOMO SAPIENS; |
| SOURCE | 9 | ORGANISM_COMMON: HUMAN; |
| SOURCE | 10 | GENUS: HOMO; |
| SOURCE | 11 | EXPRESSION_SYSTEM: SPODOPTERA FRUGIPERDA; |
| SOURCE | 12 | EXPRESSION_SYSTEM_COMMON: FALL ARMYWORM; |
| SOURCE | 13 | EXPRESSION_SYSTEM_GENUS: SPODOPTERA; |
| SOURCE | 14 | EXPRESSION_SYSTEM_VECTOR: BACULOVIRUS; |
| SOURCE | 15 | MOL_ID: 3; |
| SOURCE | 16 | ORGANISM_SCIENTIFIC: HOMO SAPIENS; |
| SOURCE | 17 | ORGANISM_COMMON: HUMAN; |
| SOURCE | 18 | GENUS: HOMO; |
| SOURCE | 19 | EXPRESSION_SYSTEM: ESCHERICHIA COLI; |
| SOURCE | 20 | EXPRESSION_SYSTEM_GENUS: ESCHERICHIA |

| | |
|---|---|
| KEYWDS | METHYLESTERASE, PHOSPHATASE, PP2A, HYDROLASE |
| EXPDTA | X-RAY DIFFRACTION |
| AUTHOR | Y. XING, Z. LI, Y. CHEN, J. STOCK, P. D. JEFFREY, Y. SHI |

| | | |
|---|---|---|
| REVDAT | 2 | 10-JUN-08 3C5W 1 JRNL |
| REVDAT | 1 | 15-APR-08 3C5W 0 |

| | | |
|---|---|---|
| JRNL | AUTH | Y. XING, Z. LI, Y. CHEN, J. B. STOCK, P. D. JEFFREY, Y. SHI |
| JRNL | TITL | STRUCTURAL MECHANISM OF DEMETHYLATION AND |
| JRNL | TITL 2 | INACTIVATION OF PROTEIN PHOSPHATASE 2A. |
| JRNL | REF | CELL(CAMBRIDGE, MASS.) V. 133 154 2008 |
| JRNL | REFN | ASTM CELLB5 US ISSN 0092-8674 |

| REMARK | 1 | | | |
|---|---|---|---|---|
| REMARK | 2 | | | |
| REMARK | 2 | RESOLUTION. 2.80 ANGSTROMS. | | |
| REMARK | 3 | | | |
| REMARK | 3 | REFINEMENT. | | |
| REMARK | 3 | PROGRAM : REFMAC 5.3.0038 | | |
| REMARK | 3 | AUTHORS : MURSHUDOV, VAGIN, DODSON | | |
| REMARK | 3 | | | |
| REMARK | 3 | REFINEMENT TARGET: MAXIMUM LIKELIHOOD | | |
| REMARK | 3 | | | |
| REMARK | 3 | DATA USED IN REFINEMENT. | | |
| REMARK | 3 | RESOLUTION RANGE HIGH | (ANGSTROMS) | : 2.80 |
| REMARK | 3 | RESOLUTION RANGE LOW | (ANGSTROMS) | : 50.00 |
| REMARK | 3 | DATA CUTOFF | (SIGMA(F)) | : 0.000 |

| | | | | |
|---|---|---|---|---|
| REMARK | 3 | COMPLETENESS FOR RANGE | (%) | : 98.6 |

| | | | |
|---|---|---|---|
| REMARK | 3 | NUMBER OF REFLECTIONS | : 19235 |
| REMARK | 3 | | |
| REMARK | 3 | FIT TO DATA USED IN REFINEMENT. | |

| REMARK | 3 | CROSS-VALIDATION METHOD | | : THROUGHOUT |
|---|---|---|---|---|
| REMARK | 3 | FREE R VALUE TEST SET SELECTION | | : RANDOM |
| REMARK | 3 | R VALUE | (WORKING + TEST SET) | : 0.198 |
| REMARK | 3 | R VALUE | (WORKING SET) | : 0.195 |

| REMARK | 3 | FREE R VALUE | | : 0.263 |
|---|---|---|---|---|
| REMARK | 3 | FREE R VALUE TEST SET SIZE | (%) | : 5.000 |

| | | | |
|---|---|---|---|
| REMARK | 3 | FREE R VALUE TEST SET COUNT | : 1018 |
| REMARK | 3 | | |
| REMARK | 3 | FIT IN THE HIGHEST RESOLUTION BIN. | |

| REMARK | 3 | TOTAL NUMBER OF BINS USED | | : 20 |
|---|---|---|---|---|
| REMARK | 3 | BIN RESOLUTION RANGE HIGH | | : 2.80 |
| REMARK | 3 | BIN RESOLUTION RANGE LOW | | : 2.87 |
| REMARK | 3 | REFLECTION IN BIN | (WORKING SET) | : 1356 |
| REMARK | 3 | BIN COMPLETENESS | (WORKING + TEST) (%) | : 95.66 |
| REMARK | 3 | BIN R VALUE | (WORKING SET) | : 0.2460 |

| | | | |
|---|---|---|---|
| REMARK | 3 | BIN FREE R VALUE SET COUNT | : 76 |
| REMARK | 3 | BIN FREE R VALUE | : 0.3280 |
| REMARK | 3 | | |

| REMARK | 3 | NUMBER OF NON-HYDROGEN ATOMS USED IN REFINEMENT. | | |
|---|---|---|---|---|
| REMARK | 3 | ALL ATOMS : 6504 | | |
| REMARK | 3 | | | |
| REMARK | 3 | B VALUES. | | |
| REMARK | 3 | FROM WILSON PLOT | (A**2) | : NULL |
| REMARK | 3 | MEAN B VALUE | (OVERALL, A**2) | : 44.74 |

| REMARK | 3 | OVERALL ANISOTROPIC B VALUE. | | |
|---|---|---|---|---|
| REMARK | 3 | B11 (A**2) : −3.32000 | | |
| REMARK | 3 | B22 (A**2) : 1.39000 | | |
| REMARK | 3 | B33 (A**2) : 0.93000 | | |
| REMARK | 3 | B12 (A**2) : 0.00000 | | |
| REMARK | 3 | B13 (A**2) : −1.39000 | | |
| REMARK | 3 | B23 (A**2) : 0.00000 | | |
| REMARK | 3 | | | |
| REMARK | 3 | ESTIMATED OVERALL COORDINATE ERROR. | | |
| REMARK | 3 | ESU BASED ON R VALUE | (A) | : NULL |
| REMARK | 3 | ESU BASED ON FREE R VALUE | (A) | : 0.438 |
| REMARK | 3 | ESU BASED ON MAXIMUM LIKELIHOOD | (A) | : 0.325 |
| REMARK | 3 | ESU FOR B VALUES BASED ON MAXIMUM LIKELIHOOD | (A**2) | : 34.630 |
| REMARK | 3 | | | |
| REMARK | 3 | CORRELATION COEFFICIENTS. | | |

| | | | |
|---|---|---|---|
| REMARK | 3 | CORRELATION COEFFICIENT FO-FC | : 0.933 |
| REMARK | 3 | CORRELATION COEFFICIENT FO-FC FREE | : 0.874 |
| REMARK | 3 | | |

| REMARK | 3 | RMS DEVIATIONS FROM IDEAL VALUES | | COUNT | RMS | WEIGHT |
|---|---|---|---|---|---|---|
| REMARK | 3 | BOND LENGTHS REFINED ATOMS | (A): | 6579 ; | 0.007 ; | 0.022 |
| REMARK | 3 | BOND LENGTHS OTHERS | (A): | NULL ; | NULL ; | NULL |
| REMARK | 3 | BOND ANGLES REFINED ATOMS | (DEGREES): | 8922 ; | 1.087 ; | 1.964 |
| REMARK | 3 | BOND ANGLES OTHERS | (DEGREES): | NULL ; | NULL ; | NULL |
| REMARK | 3 | TORSION ANGLES, PERIOD 1 | (DEGREES): | 811 ; | 5.123 ; | 5.000 |
| REMARK | 3 | TORSION ANGLES, PERIOD 2 | (DEGREES): | 297 ; | 39.029 ; | 24.276 |
| REMARK | 3 | TORSION ANGLES, PERIOD 3 | (DEGREES): | 1142 ; | 18.005 ; | 15.000 |
| REMARK | 3 | TORSION ANGLES, PERIOD 4 | (DEGREES): | 39 ; | 16.990 ; | 15.000 |
| REMARK | 3 | CHIRAL-CENTER RESTRAINTS | (A**3): | 1005 ; | 0.072 ; | 0.200 |

| | | | | | | |
|---|---|---|---|---|---|---|
| REMARK | 3 | GENERAL PLANES REFINED ATOMS | (A): | 4950 ; | 0.003 ; | 0.020 |
| REMARK | 3 | GENERAL PLANES OTHERS | (A): | NULL ; | NULL ; | NULL |
| REMARK | 3 | NON-BONDED CONTACTS REFINED ATOMS | (A): | 3244 ; | 0.194 ; | 0.200 |
| REMARK | 3 | NON-BONDED CONTACTS OTHERS | (A): | NULL ; | NULL ; | NULL |
| REMARK | 3 | NON-BONDED TORSION REFINED ATOMS | (A): | 4503 ; | 0.301 ; | 0.200 |
| REMARK | 3 | NON-BONDED TORSION OTHERS | (A): | NULL ; | NULL ; | NULL |
| REMARK | 3 | H-BOND (X . . . Y) REFINED ATOMS | (A): | 235 ; | 0.130 ; | 0.200 |
| REMARK | 3 | H-BOND (X . . . Y) OTHERS | (A): | NULL ; | NULL ; | NULL |
| REMARK | 3 | POTENTIAL METAL-ION REFINED ATOMS | (A): | NULL ; | NULL ; | NULL |
| REMARK | 3 | POTENTIAL METAL-ION OTHERS | (A): | NULL ; | NULL ; | NULL |
| REMARK | 3 | SYMMETRY VDW REFINED ATOMS | (A): | 62 ; | 0.154 ; | 0.200 |
| REMARK | 3 | SYMMETRY VDW OTHERS | (A): | NULL ; | NULL ; | NULL |
| REMARK | 3 | SYMMETRY H-BOND REFINED ATOMS | (A): | 10 ; | 0.155 ; | 0.200 |
| REMARK | 3 | SYMMETRY H-BOND OTHERS | (A): | NULL ; | NULL ; | NULL |
| REMARK | 3 | SYMMETRY METAL-ION REFINED ATOMS | (A): | NULL ; | NULL ; | NULL |
| REMARK | 3 | SYMMETRY METAL-ION OTHERS | (A): | NULL ; | NULL ; | NULL |
| REMARK | 3 | | | | | |

| REMARK | 3 | ISOTROPIC THERMAL FACTOR RESTRAINTS. | | COUNT | RMS | WEIGHT |
|---|---|---|---|---|---|---|
| REMARK | 3 | MAIN-CHAIN BOND REFINED ATOMS | (A**2): | 4175 ; | 0.249 ; | 1.500 |
| REMARK | 3 | MAIN-CHAIN BOND OTHER ATOMS | (A**2): | NULL ; | NULL ; | NULL |
| REMARK | 3 | MAIN-CHAIN ANGLE REFINED ATOMS | (A**2): | 6557 ; | 0.445 ; | 2.000 |
| REMARK | 3 | SIDE-CHAIN BOND REFINED ATOMS | (A**2): | 2730 ; | 0.633 ; | 3.000 |
| REMARK | 3 | SIDE-CHAIN ANGLE REFINED ATOMS | (A**2): | 2365 ; | 1.049 ; | 4.500 |
| REMARK | 3 | | | | | |
| REMARK | 3 | ANISOTROPIC THERMAL FACTOR RESTRAINTS. | | COUNT | RMS | WEIGHT |
| REMARK | 3 | RIGID-BOND RESTRAINTS | (A**2): | NULL ; | NULL ; | NULL |
| REMARK | 3 | SPHERICITY; FREE ATOMS | (A**2): | NULL ; | NULL ; | NULL |
| REMARK | 3 | SPHERICITY; BONDED ATOMS | (A**2): | NULL ; | NULL ; | NULL |

| REMARK | 3 | | | | | | |
|---|---|---|---|---|---|---|---|
| REMARK | 3 | NCS RESTRAINTS STATISTICS | | | | | |
| REMARK | 3 | NUMBER OF DIFFERENT NCS GROUPS : 0 | | | | | |
| REMARK | 3 | | | | | | |
| REMARK | 3 | TLS DETAILS | | | | | |
| REMARK | 3 | NUMBER OF TLS GROUPS : 5 | | | | | |
| REMARK | 3 | | | | | | |
| REMARK | 3 | TLS GROUP : 1 | | | | | |
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | A | 9 | | A | 46 |

| REMARK | 3 | ORIGIN FOR THE GROUP (A): −35.9461 2.1780 42.6351 | | | | | | |
|---|---|---|---|---|---|---|---|---|
| REMARK | 3 | T | TENSOR | | | | | |
| REMARK | 3 | | T11: | .3270 | T22: | .4444 | | |
| REMARK | 3 | | T33: | .1794 | T12: | .0837 | | |
| REMARK | 3 | | T13: | .0498 | T23: | −.0687 | | |
| REMARK | 3 | L | TENSOR | | | | | |
| REMARK | 3 | | L11: | 11.0328 | L22: | 6.9677 | | |
| REMARK | 3 | | L33: | 6.9044 | L12: | −2.4129 | | |
| REMARK | 3 | | L13: | −2.1094 | L23: | .4251 | | |
| REMARK | 3 | S | TENSOR | | | | | |
| REMARK | 3 | | S11: | .3782 | S12: | .0349 | S13: | −.1889 |
| REMARK | 3 | | S21: | .2988 | S22: | −.2210 | S23: | .9352 |
| REMARK | 3 | | S31: | .9379 | S32: | −.2054 | S33: | −.1573 |
| REMARK | 3 | | | | | | | |

| REMARK | 3 | TLS GROUP : 2 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | A | 402 | | A | 454 |

| REMARK | 3 | ORIGIN FOR THE GROUP (A): −34.4704 2.9924 28.3509 | | | | | | |
|---|---|---|---|---|---|---|---|---|
| REMARK | 3 | T | TENSOR | | | | | |
| REMARK | 3 | | T11: | −.0857 | T22: | −.0594 | | |
| REMARK | 3 | | T33: | .3068 | T12: | −.0145 | | |
| REMARK | 3 | | T13: | −.0566 | T23: | .0778 | | |
| REMARK | 3 | L | TENSOR | | | | | |
| REMARK | 3 | | L11: | 10.5488 | L22: | 1.0825 | | |
| REMARK | 3 | | L33: | 7.2277 | L12: | −2.0080 | | |
| REMARK | 3 | | L13: | −4.4543 | L23: | 1.7018 | | |
| REMARK | 3 | S | TENSOR | | | | | |
| REMARK | 3 | | S11: | −.5512 | S12: | −.7235 | S13: | .7518 |
| REMARK | 3 | | S21: | −.2593 | S22: | .3857 | S23: | .9387 |
| REMARK | 3 | | S31: | −.4504 | S32: | −.3627 | S33: | .1655 |
| REMARK | 3 | | | | | | | |

| REMARK | 3 | TLS GROUP : 3 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | A | 455 | | A | 589 |

| REMARK | 3 | ORIGIN FOR THE GROUP (A): −28.0487 −8.7261 7.2781 | | | | | | |
|---|---|---|---|---|---|---|---|---|
| REMARK | 3 | T | TENSOR | | | | | |
| REMARK | 3 | | T11: | .0515 | T22: | .3031 | | |
| REMARK | 3 | | T33: | −.1483 | T12: | −.2975 | | |
| REMARK | 3 | | T13: | −.0379 | T23: | .0178 | | |
| REMARK | 3 | L | TENSOR | | | | | |
| REMARK | 3 | | L11: | 4.4106 | L22: | 1.6241 | | |
| REMARK | 3 | | L33: | 2.2941 | L12: | .3187 | | |
| REMARK | 3 | | L13: | −2.6385 | L23: | −.6808 | | |
| REMARK | 3 | S | TENSOR | | | | | |
| REMARK | 3 | | S11: | −.3509 | S12: | 1.2696 | S13: | −.3582 |
| REMARK | 3 | | S21: | −.5932 | S22: | .1747 | S23: | .5516 |
| REMARK | 3 | | S31: | .2963 | S32: | −.7657 | S33: | .1763 |
| REMARK | 3 | | | | | | | |

| REMARK | 3 | TLS GROUP : 4 | | | | | |
|---|---|---|---|---|---|---|---|
| REMARK | 3 | NUMBER OF COMPONENTS GROUP : 1 | | | | | |
| REMARK | 3 | COMPONENTS | C | SSSEQI | TO | C | SSSEQI |
| REMARK | 3 | RESIDUE RANGE : | C | 6 | | C | 293 |

| REMARK | 3 | ORIGIN FOR THE GROUP (A): 1.5805 −2.8999 20.9402 | | | | | | |
|---|---|---|---|---|---|---|---|---|
| REMARK | 3 | T | TENSOR | | | | | |
| REMARK | 3 | | T11: | −.2067 | T22: | −.2771 | | |
| REMARK | 3 | | T33: | −.2787 | T12: | −.0494 | | |
| REMARK | 3 | | T13: | .0212 | T23: | −.0078 | | |
| REMARK | 3 | L | TENSOR | | | | | |
| REMARK | 3 | | L11: | 3.1165 | L22: | 1.8864 | | |
| REMARK | 3 | | L33: | 2.4860 | L12: | .4031 | | |
| REMARK | 3 | | L13: | −.1517 | L23: | −.2950 | | |
| REMARK | 3 | S | TENSOR | | | | | |
| REMARK | 3 | | S11: | −.0511 | S12: | .2420 | S13: | −.1052 |
| REMARK | 3 | | S21: | −.0734 | S22: | .0574 | S23: | −.0669 |
| REMARK | 3 | | S31: | .3041 | S32: | .0489 | S33: | −.0063 |
| REMARK | 3 | | | | | | | |

| REMARK | 3 | TLS GROUP : 5 | | | | | |
|---|---|---|---|---|---|---|---|
| REMARK | 3 | NUMBER OF COMPONENTS GROUP : 2 | | | | | |
| REMARK | 3 | COMPONENTS | C | SSSEQI | TO | C | SSSEQI |
| REMARK | 3 | RESIDUE RANGE : | P | 39 | | P | 376 |
| REMARK | 3 | RESIDUE RANGE : | C | 304 | | C | 309 |

| REMARK | 3 | ORIGIN FOR THE GROUP (A): 23.6125 1.2218 52.5886 | | | | | | |
|---|---|---|---|---|---|---|---|---|
| REMARK | 3 | T | TENSOR | | | | | |
| REMARK | 3 | | T11: | −.1149 | T22: | −.0634 | | |
| REMARK | 3 | | T33: | −.0996 | T12: | −.0943 | | |
| REMARK | 3 | | T13: | −.0792 | T23: | −.0248 | | |
| REMARK | 3 | L | TENSOR | | | | | |
| REMARK | 3 | | L11: | 2.3233 | L22: | 2.6104 | | |
| REMARK | 3 | | L33: | 3.7488 | L12: | −.9654 | | |
| REMARK | 3 | | L13: | .7763 | L23: | −1.8643 | | |
| REMARK | 3 | S | TENSOR | | | | | |
| REMARK | 3 | | S11: | −.1714 | S12: | −.1943 | S13: | .0232 |
| REMARK | 3 | | S21: | .4565 | S22: | −.1691 | S23: | −.4014 |
| REMARK | 3 | | S31: | −.3589 | S32: | .4543 | S33: | .3405 |
| REMARK | 3 | | | | | | | |

| REMARK | 3 | BULK SOLVENT MODELLING. | | |
|---|---|---|---|---|
| REMARK | 3 | METHOD USED: BABINET MODEL WITH MASK | | |
| REMARK | 3 | PARAMETERS FOR MASK CALCULATION | | |
| REMARK | 3 | VDW PROBE RADIUS : 1.20 | | |
| REMARK | 3 | ION PROBE RADIUS : 0.80 | | |
| REMARK | 3 | SHRINKAGE RADIUS : 0.80 | | |
| REMARK | 3 | | | |
| REMARK | 3 | OTHER REFINEMENT REMARKS: NULL | | |
| REMARK | 4 | | | |
| REMARK | 4 | 3C5W COMPLIES WITH FORMAT V. 3.1, 01-AUG-2007 | | |
| REMARK | 100 | | | |
| REMARK | 100 | THIS ENTRY HAS BEEN PROCESSED BY RCSB. | | |
| REMARK | 100 | THE RCSB ID CODE IS RCSB046380. | | |
| REMARK | 200 | | | |
| REMARK | 200 | EXPERIMENTAL DETAILS | | |
| REMARK | 200 | EXPERIMENT TYPE | | : X-RAY DIFFRACTION |
| REMARK | 200 | DATE OF DATA COLLECTION | | : 10-SEP-2007 |
| REMARK | 200 | TEMPERATURE | (KELVIN) | : 100.0 |
| REMARK | 200 | PH | | : 7.50 |
| REMARK | 200 | NUMBER OF CRYSTALS USED | | : 1 |
| REMARK | 200 | | | |
| REMARK | 200 | SYNCHROTRON | (Y/N) | : Y |
| REMARK | 200 | RADIATION SOURCE | | : NSLS |
| REMARK | 200 | BEAMLINE | | : X29A |
| REMARK | 200 | X-RAY GENERATOR MODEL | | : NULL |
| REMARK | 200 | MONOCHROMATIC OR LAUE | (M/L) | : M |
| REMARK | 200 | WAVELENGTH OR RANGE | (A) | : 1.0809 |
| REMARK | 200 | MONOCHROMATOR | | : SI (111) |
| REMARK | 200 | OPTICS | | : NULL |
| REMARK | 200 | | | |
| REMARK | 200 | DETECTOR TYPE | | : CCD |
| REMARK | 200 | DETECTOR MANUFACTURER | | : ADSC QUANTUM 315 |
| REMARK | 200 | INTENSITY-INTEGRATION SOFTWARE | | : DENZO |
| REMARK | 200 | DATA SCALING SOFTWARE | | : SCALEPACK |
| REMARK | 200 | | | |
| REMARK | 200 | NUMBER OF UNIQUE REFLECTIONS | | : 20623 |
| REMARK | 200 | RESOLUTION RANGE HIGH | (A) | : 2.800 |
| REMARK | 200 | RESOLUTION RANGE LOW | (A) | : 100.000 |
| REMARK | 200 | REJECTION CRITERIA | (SIGMA(I)) | : −3.000 |
| REMARK | 200 | | | |
| REMARK | 200 | OVERALL. | | |
| REMARK | 200 | COMPLETENESS FOR RANGE | (%) | : 98.6 |
| REMARK | 200 | DATA REDUNDANCY | | : 3.000 |
| REMARK | 200 | R MERGE | (I) | : NULL |
| REMARK | 200 | R SYM | (I) | : 0.09500 |
| REMARK | 200 | <I/SIGMA(I)> FOR THE DATA SET | | : NULL |
| REMARK | 200 | | | |
| REMARK | 200 | IN THE HIGHEST RESOLUTION SHELL. | | |
| REMARK | 200 | HIGHEST RESOLUTION SHELL, RANGE HIGH (A) : 2.80 | | |
| REMARK | 200 | HIGHEST RESOLUTION SHELL, RANGE LOW (A) : 2.90 | | |
| REMARK | 200 | COMPLETENESS FOR SHELL | (%) | : 95.7 |
| REMARK | 200 | DATA REDUNDANCY IN SHELL | | : 2.90 |
| REMARK | 200 | R MERGE FOR SHELL | (I) | : NULL |
| REMARK | 200 | R SYM FOR SHELL | (I) | : 0.40000 |
| REMARK | 200 | <I/SIGMA(I)> FOR SHELL | | : NULL |
| REMARK | 200 | | | |

| REMARK | 200 | DIFFRACTION PROTOCOL: SINGLE WAVELENGTH | | | | | |
|---|---|---|---|---|---|---|---|
| REMARK | 200 | METHOD USED TO DETERMINE THE STRUCTURE: MOLECULAR REPLACEMENT | | | | | |
| REMARK | 200 | SOFTWARE USED: PHASER | | | | | |
| REMARK | 200 | STARTING MODEL: PP2A A SUBUNIT, PP2A C SUBUNIT, PME-1 MONOMER | | | | | |
| REMARK | 200 | | | | | | |
| REMARK | 200 | REMARK: NULL | | | | | |
| REMARK | 280 | | | | | | |
| REMARK | 280 | CRYSTAL | | | | | |
| REMARK | 280 | SOLVENT CONTENT, VS (%): 43.22 | | | | | |
| REMARK | 280 | MATTHEWS COEFFICIENT, VM (ANGSTROMS**3/DA): 2.17 | | | | | |
| REMARK | 280 | | | | | | |
| REMARK | 280 | CRYSTALLIZATION CONDITIONS: 25% W/V PEG3350, 100 MM AMMONIUM | | | | | |
| REMARK | 280 | CITRATE, AND 5 MM DTT, PH 7.5, VAPOR DIFFUSION, HANGING DROP, | | | | | |
| REMARK | 280 | TEMPERATURE 290K | | | | | |
| REMARK | 290 | | | | | | |
| REMARK | 290 | CRYSTALLOGRAPHIC SYMMETRY | | | | | |
| REMARK | 290 | SYMMETRY OPERATORS FOR SPACE GROUP: C 1 2 1 | | | | | |
| REMARK | 290 | | | | | | |
| REMARK | 290 | SYMOP | | SYMMETRY | | | |
| REMARK | 290 | NNNMMM | | OPERATOR | | | |
| REMARK | 290 | 1555 | | X, Y, Z | | | |
| REMARK | 290 | 2555 | | −X, Y, −Z | | | |
| REMARK | 290 | 3555 | | ½ + X, ½ + Y, Z | | | |
| REMARK | 290 | 4555 | | ½ − X, ½ + Y, −Z | | | |
| REMARK | 290 | | | | | | |
| REMARK | 290 | WHERE NNN -> OPERATOR NUMBER | | | | | |
| REMARK | 290 | MMM -> TRANSLATION VECTOR | | | | | |
| REMARK | 290 | | | | | | |
| REMARK | 290 | CRYSTALLOGRAPHIC SYMMETRY TRANSFORMATIONS | | | | | |
| REMARK | 290 | THE FOLLOWING TRANSFORMATIONS OPERATE ON THE ATOM/HETATM | | | | | |
| REMARK | 290 | RECORDS IN THIS ENTRY TO PRODUCE CRYSTALLOGRAPHICALLY | | | | | |
| REMARK | 290 | RELATED MOLECULES. | | | | | |
| REMARK | 290 | SMTRY1 1 1.000000 0.000000 0.000000 0.00000 | | | | | |
| REMARK | 290 | SMTRY2 1 0.000000 1.000000 0.000000 0.00000 | | | | | |
| REMARK | 290 | SMTRY3 1 0.000000 0.000000 1.000000 0.00000 | | | | | |
| REMARK | 290 | SMTRY1 2 −1.000000 0.000000 0.000000 0.00000 | | | | | |
| REMARK | 290 | SMTRY2 2 0.000000 1.000000 0.000000 0.00000 | | | | | |
| REMARK | 290 | SMTRY3 2 0.000000 0.000000 −1.000000 0.00000 | | | | | |
| REMARK | 290 | SMTRY1 3 1.000000 0.000000 0.000000 64.64600 | | | | | |
| REMARK | 290 | SMTRY2 3 0.000000 1.000000 0.000000 27.41950 | | | | | |
| REMARK | 290 | SMTRY3 3 0.000000 0.000000 1.000000 0.00000 | | | | | |
| REMARK | 290 | SMTRY1 4 −1.000000 0.000000 0.000000 64.64600 | | | | | |
| REMARK | 290 | SMTRY2 4 0.000000 1.000000 0.000000 27.41950 | | | | | |
| REMARK | 290 | SMTRY3 4 0.000000 0.000000 −1.000000 0.00000 | | | | | |
| REMARK | 290 | | | | | | |
| REMARK | 290 | REMARK: NULL | | | | | |
| REMARK | 300 | | | | | | |
| REMARK | 300 | BIOMOLECULE: 1 | | | | | |
| REMARK | 300 | SEE REMARK 350 FOR THE AUTHOR PROVIDED AND/OR PROGRAM | | | | | |
| REMARK | 300 | GENERATED ASSEMBLY INFORMATION FOR THE STRUCTURE IN | | | | | |
| REMARK | 300 | THIS ENTRY. THE REMARK MAY ALSO PROVIDE INFORMATION ON | | | | | |
| REMARK | 300 | BURIED SURFACE AREA. | | | | | |
| REMARK | 350 | | | | | | |
| REMARK | 350 | COORDINATES FOR A COMPLETE MULTIMER REPRESENTING THE KNOWN | | | | | |
| REMARK | 350 | BIOLOGICALLY SIGNIFICANT OLIGOMERIZATION STATE OF THE | | | | | |
| REMARK | 350 | MOLECULE CAN BE GENERATED BY APPLYING BIOMT TRANSFORMATIONS | | | | | |
| REMARK | 350 | GIVEN BELOW. BOTH NON-CRYSTALLOGRAPHIC AND | | | | | |
| REMARK | 350 | CRYSTALLOGRAPHIC OPERATIONS ARE GIVEN. | | | | | |
| REMARK | 350 | | | | | | |
| REMARK | 350 | BIOMOLECULE: 1 | | | | | |
| REMARK | 350 | AUTHOR DETERMINED BIOLOGICAL UNIT: TRIMERIC | | | | | |
| REMARK | 350 | SOFTWARE DETERMINED QUATERNARY STRUCTURE: TRIMERIC | | | | | |
| REMARK | 350 | SOFTWARE USED: PISA | | | | | |
| REMARK | 350 | TOTAL BURIED SURFACE AREA: 4920 ANGSTROM**2 | | | | | |
| REMARK | 350 | APPLY THE FOLLOWING TO CHAINS: A, C, P | | | | | |
| REMARK | 350 | BIOMT1 | 1 | 1.000000 | 0.000000 | 0.000000 | 0.00000 |
| REMARK | 350 | BIOMT2 | 1 | 0.000000 | 1.000000 | 0.000000 | 0.00000 |
| REMARK | 350 | BIOMT3 | 1 | 0.000000 | 0.000000 | 1.000000 | 0.00000 |
| REMARK | 465 | | | | | | |

| REMARK | 465 | MISSING RESIDUES | | | |
|---|---|---|---|---|---|
| REMARK | 465 | THE FOLLOWING RESIDUES WERE NOT LOCATED IN THE | | | |
| REMARK | 465 | EXPERIMENT. (M = MODEL NUMBER; RES = RESIDUE NAME; C = CHAIN | | | |
| REMARK | 465 | IDENTIFIER; SSEQ = SEQUENCE NUMBER; I = INSERTION CODE.) | | | |
| REMARK | 465 | | | | |
| REMARK | 465 | M | RES | C | SSEQI |
| REMARK | 465 | | GLY | A | 5 |
| REMARK | 465 | | SER | A | 6 |
| REMARK | 465 | | HIS | A | 7 |
| REMARK | 465 | | MET | A | 8 |
| REMARK | 465 | | GLY | C | 0 |
| REMARK | 465 | | MET | C | 1 |
| REMARK | 465 | | ASP | C | 2 |
| REMARK | 465 | | GLU | C | 3 |
| REMARK | 465 | | LYS | C | 4 |
| REMARK | 465 | | VAL | C | 5 |
| REMARK | 465 | | ARG | C | 294 |
| REMARK | 465 | | ARG | C | 295 |
| REMARK | 465 | | GLY | C | 296 |
| REMARK | 465 | | GLU | C | 297 |
| REMARK | 465 | | PRO | C | 298 |
| REMARK | 465 | | HIS | C | 299 |
| REMARK | 465 | | VAL | C | 300 |
| REMARK | 465 | | THR | C | 301 |
| REMARK | 465 | | ARG | C | 302 |
| REMARK | 465 | | ARG | C | 303 |
| REMARK | 465 | | GLY | P | 35 |
| REMARK | 465 | | SER | P | 36 |
| REMARK | 465 | | HIS | P | 37 |
| REMARK | 465 | | MET | P | 38 |
| REMARK | 465 | | GLU | P | 239 |
| REMARK | 465 | | GLY | P | 240 |
| REMARK | 465 | | GLY | P | 377 |
| REMARK | 465 | | GLY | P | 378 |
| REMARK | 465 | | PHE | P | 379 |
| REMARK | 465 | | GLN | P | 380 |
| REMARK | 465 | | CYS | P | 381 |
| REMARK | 465 | | VAL | P | 382 |
| REMARK | 465 | | PHE | P | 383 |
| REMARK | 465 | | PRO | P | 384 |
| REMARK | 465 | | GLY | P | 385 |
| REMARK | 465 | | CYS | P | 386 |
| REMARK | 500 | | | | |

| REMARK | 500 | GEOMETRY AND STEREOCHEMISTRY | | | | | |
|---|---|---|---|---|---|---|---|
| REMARK | 500 | SUBTOPIC: TORSION ANGLES | | | | | |
| REMARK | 500 | | | | | | |
| REMARK | 500 | TORSION ANGLES OUTSIDE THE EXPECTED RAMACHANDRAN REGIONS: | | | | | |
| REMARK | 500 | (M = MODEL NUMBER; RES = RESIDUE NAME; C = CHAIN IDENTIFIER; | | | | | |
| REMARK | 500 | SSEQ = SEQUENCE NUMBER; I = INSERTION CODE). | | | | | |
| REMARK | 500 | | | | | | |
| REMARK | 500 | STANDARD TABLE: | | | | | |
| REMARK | 500 | FORMAT: (10X, I3, 1X, A3, 1X, A1, I4, A1, 4X, F7.2, 3X, F7.2) | | | | | |
| REMARK | 500 | | | | | | |
| REMARK | 500 | EXPECTED VALUES: GJ KLEYWEGT AND TA JONES (1996). PHI/PSI- | | | | | |
| REMARK | 500 | CHOLOGY: RAMACHANDRAN REVISITED. STRUCTURE 4, 1395-1400 | | | | | |
| REMARK | 500 | | | | | | |
| REMARK | 500 | M | RES | CSSEQI | | PSI | PHI |
| REMARK | 500 | | LYS | A | 34 | −14.48 | −159.53 |
| REMARK | 500 | | LEU | A | 42 | 80.86 | −168.34 |
| REMARK | 500 | | LEU | A | 404 | −33.18 | −148.07 |
| REMARK | 500 | | GLU | A | 441 | −98.38 | −65.77 |
| REMARK | 500 | | SER | A | 587 | 7.49 | 59.11 |
| REMARK | 500 | | ASP | C | 88 | −113.24 | −153.79 |
| REMARK | 500 | | TYR | C | 91 | −64.70 | −96.20 |
| REMARK | 500 | | ASN | C | 139 | −158.96 | −101.87 |
| REMARK | 500 | | GLN | C | 162 | −45.20 | −131.29 |
| REMARK | 500 | | ALA | C | 240 | −131.42 | −125.03 |
| REMARK | 500 | | HIS | C | 241 | −31.72 | 78.98 |
| REMARK | 500 | | TYR | C | 267 | 18.76 | 59.43 |
| REMARK | 500 | | ARG | C | 268 | −16.63 | −142.78 |
| REMARK | 500 | | ASP | C | 279 | −166.68 | −78.63 |
| REMARK | 500 | | ASP | C | 306 | 53.33 | −98.11 |
| REMARK | 500 | | ASN | P | 60 | −118.74 | −139.74 |
| REMARK | 500 | | ARG | P | 68 | 112.70 | 81.62 |
| REMARK | 500 | | HIS | P | 87 | −156.95 | −105.94 |
| REMARK | 500 | | SER | P | 114 | −5.57 | 66.88 |
| REMARK | 500 | | TYR | P | 143 | −72.73 | −127.07 |
| REMARK | 500 | | ALA | P | 156 | −115.60 | 39.27 |
| REMARK | 500 | | ARG | P | 201 | 151.88 | −48.59 |
| REMARK | 500 | | GLU | P | 290 | 79.34 | −67.37 |
| REMARK | 500 | | PRO | P | 313 | 48.01 | −79.23 |
| REMARK | 500 | | | | | | |

| REMARK | 500 | REMARK: NULL | | | | | | | |
|---|---|---|---|---|---|---|---|---|---|
| REMARK | 900 | | | | | | | | |
| REMARK | 900 | RELATED ENTRIES | | | | | | | |
| REMARK | 900 | RELATED ID: 3C5V RELATED DB: PDB | | | | | | | |
| REMARK | 900 | PME-1 MONOMER | | | | | | | |
| REMARK | 999 | | | | | | | | |
| REMARK | 999 | SEQUENCE | | | | | | | |
| REMARK | 999 | CHAIN A HAS 47-399 RESIDUES DELETION AND CHAIN P HAS 239- | | | | | | | |
| REMARK | 999 | 283 LOOP REPLACED BY EGK. THE PROTEIN CONSTRUCT (CHAIN P) | | | | | | | |
| REMARK | 999 | IS A NON-CATALYTIC MUTANT WITH SER156 REPLACED BY ALA. | | | | | | | |
| DBREF | 3C5W | A | 9 | 46 | UNP | P30153 | 2AAA_HUMAN | 9 | 46 |
| DBREF | 3C5W | A | 400 | 589 | UNP | P30153 | 2AAA_HUMAN | 400 | 589 |
| DEREF | 3C5W | C | 1 | 309 | UNP | P67775 | PP2AA_HUMAN | 1 | 309 |
| DBREF | 3C5W | P | 39 | 238 | UNP | Q9Y570 | PPME1_HUMAN | 39 | 238 |
| DBREF | 3C5W | P | 284 | 386 | UNP | Q9Y570 | PPME1_HUMAN | 284 | 386 |

| | | | | | | | | | | |
|---|---|---|---|---|---|---|---|---|---|---|
| SEQADV | 3C5W | GLY | A | 5 | UNP | P30153 | | | EXPRESSION | TAG |
| SEQADV | 3C5W | SER | A | 6 | UNP | P30153 | | | EXPRESSION | TAG |
| SEQADV | 3C5W | HIS | A | 7 | UNP | P30153 | | | EXPRESSION | TAG |
| SEQADV | 3C5W | MET | A | 8 | UNP | P30153 | | | EXPRESSION | TAG |
| SEQADV | 3C5W | GLY | C | 0 | UNP | P67775 | | | EXPRESSION | TAG |
| SEQADV | 3C5W | GLY | P | 35 | UNP | Q9Y570 | | | EXPRESSION | TAG |
| SEQADV | 3C5W | SER | P | 36 | UNP | Q9Y570 | | | EXPRESSION | TAG |
| SEQADV | 3C5W | HIS | P | 37 | UNP | Q9Y570 | | | EXPRESSION | TAG |
| SEQADV | 3C5W | MET | P | 38 | UNP | Q9Y570 | | | EXPRESSION | TAG |
| SEQADV | 3C5W | ALA | P | 156 | UNP | Q9Y570 | SER | 156 | SEE REMARK | 999 |
| SEQADV | 3C5W | GLU | P | 239 | UNP | Q9Y570 | | | SEE REMARK | 999 |
| SEQADV | 3C5W | GLY | P | 240 | UNP | Q9Y570 | | | SEE REMARK | 999 |
| SEQADV | 3C5W | LYS | P | 283 | UNP | Q9Y570 | | | SEE REMARK | 999 |

| | | | | | | | | | | | | | | | | |
|---|---|---|---|---|---|---|---|---|---|---|---|---|---|---|---|---|
| SEQRES | 1 | A | 232 | GLY | SER | HIS | MET | SER | LEU | TYR | PRO | ILE | ALA | VAL | LEU | ILE |
| SEQRES | 2 | A | 232 | ASP | GLU | LEU | ARG | ASN | GLU | ASP | VAL | GLN | LEU | ARG | LEU | ASN |
| SEQRES | 3 | A | 232 | SER | ILE | LYS | LYS | LEU | SER | THR | ILE | ALA | LEU | ALA | LEU | GLY |
| SEQRES | 4 | A | 232 | VAL | GLU | ARG | LEU | SER | GLN | SER | LEU | LEU | PRO | ALA | ILE | VAL |
| SEQRES | 5 | A | 232 | GLU | LEU | ALA | GLU | ASP | ALA | LYS | TRP | ARG | VAL | ARG | LEU | ALA |
| SEQRES | 6 | A | 232 | ILE | ILE | GLU | TYR | MET | PRO | LEU | LEU | ALA | GLY | GLN | LEU | GLY |
| SEQRES | 7 | A | 232 | VAL | GLU | PHE | PHE | ASP | GLU | LYS | LEU | ASN | SER | LEU | CYS | MET |
| SEQRES | 8 | A | 232 | ALA | TRP | LEU | VAL | ASP | HIS | VAL | TYR | ALA | ILE | ARG | GLU | ALA |
| SEQRES | 9 | A | 232 | ALA | THR | SER | ASN | LEU | LYS | LYS | LEU | VAL | GLU | LYS | PHE | GLY |
| SEQRES | 10 | A | 232 | LYS | GLU | TRP | ALA | HIS | ALA | THR | ILE | ILE | PRO | LYS | VAL | LEU |
| SEQRES | 11 | A | 232 | ALA | MET | SER | GLY | ASP | PRO | ASN | TYR | LEU | HIS | ARG | MET | THR |
| SEQRES | 12 | A | 232 | THR | LEU | PHE | CYS | ILE | ASN | VAL | LEU | SER | GLU | VAL | CYS | GLY |
| SEQRES | 13 | A | 232 | GLN | ASP | ILE | THR | THR | LYS | HIS | MET | LEU | PRO | THR | VAL | LEU |
| SEQRES | 14 | A | 232 | ARG | MET | ALA | GLY | ASP | PRO | VAL | ALA | ASN | VAL | ARG | PHE | ASN |
| SEQRES | 15 | A | 232 | VAL | ALA | LYS | SER | LEU | GLN | LYS | ILE | GLY | PRO | ILE | LEU | ASP |
| SEQRES | 16 | A | 232 | ASN | SER | THR | LEU | GLN | SER | GLU | VAL | LYS | PRO | ILE | LEU | GLU |
| SEQRES | 17 | A | 232 | LYS | LEU | THR | GLN | ASP | GLN | ASP | VAL | ASP | VAL | LYS | TYR | PHE |
| SEQRES | 18 | A | 232 | ALA | GLN | GLU | ALA | LEU | THR | VAL | LEU | SER | LEU | ALA | | |
| SEQRES | 1 | C | 310 | GLY | MET | ASP | GLU | LYS | VAL | PHE | THR | LYS | GLU | LEU | ASP | GLN |
| SEQRES | 2 | C | 310 | TRP | ILE | GLU | GLN | LEU | ASN | GLU | CYS | LYS | GLN | LEU | SER | GLU |
| SEQRES | 3 | C | 310 | SER | GLN | VAL | LYS | SER | LEU | CYS | GLU | LYS | ALA | LYS | GLU | ILE |
| SEQRES | 4 | C | 310 | LEU | THR | LYS | GLU | SER | ASN | VAL | GLN | GLU | VAL | ARG | CYS | PRO |
| SEQRES | 5 | C | 310 | VAL | THR | VAL | CYS | GLY | ASP | VAL | HIS | GLY | GLN | PHE | HIS | ASP |
| SEQRES | 6 | C | 310 | LEU | MET | GLU | LEU | PHE | ARG | ILE | GLY | GLY | LYS | SER | PRO | ASP |
| SEQRES | 7 | C | 310 | THR | ASN | TYR | LEU | PHE | MET | GLY | ASP | TYR | VAL | ASP | ARG | GLY |
| SEQRES | 8 | C | 310 | TYR | TYR | SER | VAL | GLU | THR | VAL | THR | LEU | LEU | VAL | ALA | LEU |
| SEQRES | 9 | C | 310 | LYS | VAL | ARG | TYR | ARG | GLU | ARG | ILE | THR | ILE | LEU | ARG | GLY |
| SEQRES | 10 | C | 310 | ASN | HIS | GLU | SER | ARG | GLN | ILE | THR | GLN | VAL | TYR | GLY | PHE |
| SEQRES | 11 | C | 310 | TYR | ASP | GLU | CYS | LEU | ARG | LYS | TYR | GLY | ASN | ALA | ASN | VAL |
| SEQRES | 12 | C | 310 | TRP | LYS | TYR | PHE | THR | ASP | LEU | PHE | ASP | TYR | LEU | PRO | LEU |
| SEQRES | 13 | C | 310 | THR | ALA | LEU | VAL | ASP | GLY | GLN | ILE | PHE | CYS | LEU | HIS | GLY |
| SEQRES | 14 | C | 310 | GLY | LEU | SER | PRO | SER | ILE | ASP | THR | LEU | ASP | HIS | ILE | ARG |
| SEQRES | 15 | C | 310 | ALA | LEU | ASP | ARG | LEU | GLN | GLU | VAL | PRO | HIS | GLU | GLY | PRO |
| SEQRES | 16 | C | 310 | MET | CYS | ASP | LEU | LEU | TRP | SER | ASP | PRO | ASP | ASP | ARG | GLY |
| SEQRES | 17 | C | 310 | GLY | TRP | GLY | ILE | SER | PRO | ARG | GLY | ALA | GLY | TYR | THR | PHE |
| SEQRES | 18 | C | 310 | GLY | GLN | ASP | ILE | SER | GLU | THR | PHE | ASN | HIS | ALA | ASN | GLY |
| SEQRES | 19 | C | 310 | LEU | THR | LEU | VAL | SER | ARG | ALA | HIS | GLN | LEU | VAL | MET | GLU |
| SEQRES | 20 | C | 310 | GLY | TYR | ASN | TRP | CYS | HIS | ASP | ARG | ASN | VAL | VAL | THR | ILE |
| SEQRES | 21 | C | 310 | PHE | SER | ALA | PRO | ASN | TYR | CYS | TYR | ARG | CYS | GLY | ASN | GLN |
| SEQRES | 22 | C | 310 | ALA | ALA | ILE | MET | GLU | LEU | ASP | ASP | THR | LEU | LYS | TYR | SER |
| SEQRES | 23 | C | 310 | PHE | LEU | GLN | PHE | ASP | PRO | ALA | PRO | ARG | ARG | GLY | GLU | PRO |
| SEQRES | 24 | C | 310 | HIS | VAL | THR | ARG | ARG | THR | PRO | ASP | TYR | PHE | LEU | | |
| SEQRES | 1 | P | 310 | GLY | SER | HIS | MET | ARG | ASP | PHE | SER | PRO | VAL | PRO | TRP | SER |
| SEQRES | 2 | P | 310 | GLN | TYR | PHE | GLU | SER | MET | GLU | ASP | VAL | GLU | VAL | GLU | ASN |
| SEQRES | 3 | P | 310 | GLU | THR | GLY | LYS | ASP | THR | PHE | ARG | VAL | TYR | LYS | SER | GLY |
| SEQRES | 4 | P | 310 | SER | GLU | GLY | PRO | VAL | LEU | LEU | LEU | LEU | HIS | GLY | GLY | GLY |
| SEQEES | 5 | P | 310 | HIS | SER | ALA | LEU | SER | TRP | ALA | VAL | PHE | THR | ALA | ALA | ILE |
| SEQEES | 6 | P | 310 | ILE | SER | ARG | VAL | GLN | CYS | ARG | ILE | VAL | ALA | LEU | ASP | LEU |
| SEQRES | 7 | P | 310 | ARG | SER | HIS | GLY | GLU | THR | LYS | VAL | LYS | ASN | PRO | GLU | ASP |
| SEQEES | 8 | P | 310 | LEU | SER | ALA | GLU | THR | MET | ALA | LYS | ASP | VAL | GLY | ASN | VAL |
| SEQEES | 9 | P | 310 | VAL | GLU | ALA | MET | TYR | GLY | ASP | LEU | PRO | PRO | PRO | ILE | MET |
| SEQEES | 10 | P | 310 | LEU | ILE | GLY | HIS | ALA | MET | GLY | GLY | ALA | ILE | ALA | VAL | HIS |
| SEQRES | 11 | P | 310 | THR | ALA | SER | SER | ASN | LEU | VAL | PRO | SER | LEU | LEU | GLY | LEU |
| SEQRES | 12 | P | 310 | CYS | MET | ILE | ASP | VAL | VAL | GLU | GLY | THR | ALA | MET | ASP | ALA |
| SEQRES | 13 | P | 310 | LEU | ASN | SER | MET | GLN | ASN | PHE | LEU | ARG | GLY | ARG | PRO | LYS |
| SEQEES | 14 | P | 310 | THR | PHE | LYS | SER | LEU | GLU | ASN | ALA | ILE | GLU | TRP | SER | VAL |
| SEQEES | 15 | P | 310 | LYS | SER | GLY | GLN | ILE | ARG | ASN | LEU | GLU | SER | ALA | ARG | VAL |
| SEQRES | 16 | P | 310 | SER | MET | VAL | GLY | GLN | VAL | LYS | GLN | CYS | GLU | GLY | LYS | PRO |
| SEQRES | 17 | P | 310 | TYR | THR | TRP | ARG | ILE | GLU | LEU | ALA | LYS | THR | GLU | LYS | TYR |
| SEQRES | 18 | P | 310 | TRP | ASP | GLY | TRP | PHE | ARG | GLY | LEU | SER | ASN | LEU | PHE | LEU |
| SEQEES | 19 | P | 310 | SER | CYS | PRO | ILE | PRO | LYS | LEU | LEU | LEU | LEU | ALA | GLY | VAL |
| SEQRES | 20 | P | 310 | ASP | ARG | LEU | ASP | LYS | ASP | LEU | THR | ILE | GLY | GLN | MET | GLN |
| SEQEES | 21 | P | 310 | GLY | LYS | PHE | GLN | MET | GLN | VAL | LEU | PRO | GLN | CYS | GLY | HIS |
| SEQRES | 22 | P | 310 | ALA | VAL | HIS | GLU | ASP | ALA | PRO | ASP | LYS | VAL | ALA | GLU | ALA |
| SEQRES | 23 | P | 310 | VAL | ALA | THR | PHE | LEU | ILE | ARG | HIS | ARG | PHE | ALA | GLU | PRO |
| SEQRES | 24 | P | 310 | ILE | GLY | GLY | PHE | GLN | CYS | VAL | PHE | PRO | GLY | CYS | | |

| | | | |
|---|---|---|---|
| FORMUL | 4 | HOH | *62 (H2O) |

| | | | | | | | | | | |
|---|---|---|---|---|---|---|---|---|---|---|
| HELIX | 1 | 1 | TYR | A | 11 | ARG | A | 21 | 1 | 11 |
| HELIX | 2 | 2 | ASP | A | 24 | LYS | A | 33 | 1 | 10 |
| HELIX | 3 | 3 | LYS | A | 34 | GLY | A | 43 | 1 | 10 |
| HELIX | 4 | 4 | GLU | A | 45 | SER | A | 403 | 1 | 6 |
| HELIX | 5 | 5 | LEU | A | 404 | ALA | A | 412 | 1 | 9 |
| HELIX | 6 | 6 | LYS | A | 416 | GLU | A | 425 | 1 | 10 |
| HELIX | 7 | 7 | TYR | A | 426 | GLY | A | 435 | 1 | 10 |
| HELIX | 8 | 8 | VAL | A | 436 | PHE | A | 439 | 5 | 4 |
| HELIX | 9 | 9 | LYS | A | 442 | LEU | A | 451 | 1 | 10 |
| HELIX | 10 | 10 | VAL | A | 455 | GLY | A | 474 | 1 | 20 |
| HELIX | 11 | 11 | GLY | A | 474 | THR | A | 481 | 1 | 8 |
| HELIX | 12 | 12 | THR | A | 481 | MET | A | 489 | 1 | 9 |
| HELIX | 13 | 13 | SER | A | 490 | ASP | A | 492 | 5 | 3 |
| HELIX | 14 | 14 | ASN | A | 494 | MET | A | 521 | 1 | 28 |
| HELIX | 15 | 15 | MET | A | 521 | MET | A | 528 | 1 | 8 |
| HELIX | 16 | 16 | ALA | A | 529 | ASP | A | 531 | 5 | 3 |
| HELIX | 17 | 17 | VAL | A | 533 | GLY | A | 548 | 1 | 16 |
| HELIX | 18 | 18 | PRO | A | 549 | LEU | A | 551 | 5 | 3 |
| HELIX | 19 | 19 | ASP | A | 552 | GLU | A | 559 | 1 | 8 |
| HELIX | 20 | 20 | VAL | A | 560 | GLN | A | 569 | 1 | 10 |
| HELIX | 21 | 21 | ASP | A | 572 | LEU | A | 586 | 1 | 15 |
| HELIX | 22 | 22 | PHE | C | 6 | ASN | C | 18 | 1 | 13 |
| HELIX | 23 | 23 | SER | C | 24 | LYS | C | 41 | 1 | 18 |
| HELIX | 24 | 24 | GLN | C | 61 | GLY | C | 73 | 1 | 13 |
| HELIX | 25 | 25 | TYR | C | 92 | TYR | C | 107 | 1 | 16 |
| HELIX | 26 | 26 | SER | C | 120 | GLY | C | 128 | 1 | 9 |
| HELIX | 27 | 27 | GLY | C | 128 | GLY | C | 138 | 1 | 11 |
| HELIX | 28 | 28 | ALA | C | 140 | ASP | C | 151 | 1 | 12 |
| HELIX | 29 | 29 | THR | C | 176 | LEU | C | 183 | 1 | 8 |
| HELIX | 30 | 30 | GLY | C | 193 | SER | C | 201 | 1 | 9 |
| HELIX | 31 | 31 | GLY | C | 221 | ASN | C | 232 | 1 | 12 |
| HELIX | 32 | 32 | ASN | C | 264 | ARG | C | 268 | 5 | 5 |
| HELIX | 33 | 33 | PRO | P | 45 | TYR | P | 49 | 5 | 5 |
| HELIX | 34 | 34 | SER | P | 88 | SER | P | 91 | 5 | 4 |
| HELIX | 35 | 35 | TRP | P | 92 | SER | P | 101 | 1 | 10 |
| HELIX | 36 | 36 | SER | P | 127 | TYR | P | 143 | 1 | 17 |
| HELIX | 37 | 37 | ALA | P | 156 | SER | P | 168 | 1 | 13 |
| HELIX | 38 | 38 | VAL | P | 183 | ARG | P | 199 | 1 | 17 |
| HELIX | 39 | 39 | SER | P | 207 | SER | P | 218 | 1 | 12 |
| HELIX | 40 | 40 | ASN | P | 223 | MET | P | 231 | 1 | 9 |
| HELIX | 41 | 41 | GLU | P | 290 | LYS | P | 296 | 5 | 7 |
| HELIX | 42 | 42 | TYR | P | 297 | ARG | P | 303 | 1 | 7 |
| HELIX | 43 | 43 | GLY | P | 304 | CYS | P | 312 | 1 | 9 |
| HELIX | 44 | 44 | GLY | P | 322 | LEU | P | 326 | 5 | 5 |
| HELIX | 45 | 45 | ASP | P | 327 | GLN | P | 336 | 1 | 10 |
| HELIX | 46 | 46 | ALA | P | 350 | ALA | P | 355 | 1 | 6 |
| HELIX | 47 | 47 | ALA | P | 355 | HIS | P | 370 | 1 | 16 |

| | | | | | | | | | | | | | | | | | | |
|---|---|---|---|---|---|---|---|---|---|---|---|---|---|---|---|---|---|---|
| SHEET | 1 | A | 6 | VAL | C | 45 | VAL | C | 48 | 0 | | | | | | | | |
| SHEET | 2 | A | 6 | THR | C | 156 | VAL | C | 159 | 1 | O | LEU | C | 158 | N | VAL | C | 48 |
| SHEET | 3 | A | 6 | ILE | C | 163 | LEU | C | 166 | −1 | O | CYS | C | 165 | N | ALA | C | 157 |
| SHEET | 4 | A | 6 | LEU | C | 236 | ARG | C | 239 | 1 | O | SER | C | 238 | N | PHE | C | 164 |
| SHEET | 5 | A | 6 | VAL | C | 256 | ILE | C | 259 | 1 | O | ILE | C | 259 | N | ARG | C | 239 |
| SHEET | 6 | A | 6 | TYR | C | 248 | CYS | C | 251 | −1 | N | ASN | C | 249 | O | THR | C | 258 |
| SHEET | 1 | B | 5 | ILE | C | 111 | ILE | C | 113 | 0 | | | | | | | | |
| SHEET | 2 | B | 5 | TYR | C | 80 | PHE | C | 82 | 1 | N | PHE | C | 82 | O | THR | C | 112 |
| SHEET | 3 | B | 5 | VAL | C | 52 | CYS | C | 55 | 1 | N | CYS | C | 55 | O | LEU | C | 81 |
| SHEET | 4 | B | 5 | ALA | C | 273 | LEU | C | 278 | −1 | O | MET | C | 276 | N | VAL | C | 54 |
| SHEET | 5 | B | 5 | TYR | C | 284 | PHE | C | 289 | −1 | O | PHE | C | 289 | N | ALA | C | 273 |
| SHEET | 1 | C | 3 | ASP | C | 202 | PRO | C | 203 | 0 | | | | | | | | |
| SHEET | 2 | C | 3 | TYR | C | 218 | PHE | C | 220 | 1 | O | PHE | C | 220 | N | ASP | C | 202 |
| SHEET | 3 | C | 3 | TRP | C | 209 | ILE | C | 211 | −1 | N | GLY | C | 210 | O | THR | C | 219 |
| SHEET | 1 | D | 8 | SER | P | 52 | GLU | P | 54 | 0 | | | | | | | | |
| SHEET | 2 | D | 8 | VAL | P | 69 | SER | P | 72 | −1 | O | VAL | P | 69 | N | GLU | P | 54 |
| SHEET | 3 | D | 8 | ARG | P | 106 | LEU | P | 110 | −1 | O | ALA | P | 109 | N | TYR | P | 70 |
| SHEET | 4 | D | 8 | VAL | P | 78 | LEU | P | 82 | 1 | N | LEU | P | 79 | O | ARG | P | 106 |
| SHEET | 5 | D | 8 | ILE | P | 150 | HIS | P | 155 | 1 | O | HIS | P | 155 | N | LEU | P | 82 |
| SHEET | 6 | D | 8 | LEU | P | 174 | ILE | P | 180 | 1 | O | ILE | P | 180 | N | GLY | P | 154 |
| SHEET | 7 | D | 8 | LYS | P | 316 | LEU | P | 320 | 1 | O | LEU | P | 317 | N | LEU | P | 177 |
| SHEET | 8 | D | 8 | GLN | P | 340 | VAL | P | 343 | 1 | O | GLN | P | 342 | N | LEU | P | 320 |
| SHEET | 1 | E | 2 | GLU | P | 57 | GLU | P | 59 | 0 | | | | | | | | |
| SHEET | 2 | E | 2 | LYS | P | 64 | THR | P | 66 | −1 | O | ASP | P | 65 | N | VAL | P | 58 |
| SHEET | 1 | F | 2 | VAL | P | 103 | GLN | P | 104 | 0 | | | | | | | | |
| SHEET | 2 | F | 2 | GLU | P | 374 | PRO | P | 375 | −1 | O | GLU | P | 374 | N | GLN | P | 104 |
| SHEET | 1 | G | 3 | THR | P | 204 | PHE | P | 205 | 0 | | | | | | | | |
| SHEET | 2 | G | 3 | TYR | P | 285 | TRP | P | 287 | −1 | O | TYR | P | 285 | N | PHE | P | 205 |
| SHEET | 3 | G | 3 | VAL | P | 235 | GLN | P | 237 | −1 | N | LYS | P | 236 | O | THR | P | 286 |

| | | | | | | | | | |
|---|---|---|---|---|---|---|---|---|---|
| CISPEP | 1 | CYS | C | 50 | PRO | C | 51 | 0 | 1.59 |
| CISPEP | 2 | SER | C | 75 | PRO | C | 76 | 0 | 6.39 |

| | | | | | | | | |
|---|---|---|---|---|---|---|---|---|
| CRYST1 | 129.292 | 54.839 | 125.146 | 90.00 | 110.87 | 90.00 | C 1 2 1 | 4 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.007734 | 0.000000 | 0.002949 | 0.00000 |
| SCALE2 | 0.000000 | 0.018235 | 0.000000 | 0.00000 |
| SCALE3 | 0.000000 | 0.000000 | 0.008552 | 0.00000 |

| | | | | | | | | | | | |
|---|---|---|---|---|---|---|---|---|---|---|---|
| ATOM | 1 | N | SER | A | 9 | −46.162 | 1.291 | 51.671 | 1.00 | 47.62 | N |
| ATOM | 2 | CA | SER | A | 9 | −45.958 | 0.774 | 50.284 | 1.00 | 47.65 | C |
| ATOM | 3 | C | SER | A | 9 | −45.940 | 1.901 | 49.245 | 1.00 | 47.63 | C |
| ATOM | 4 | O | SER | A | 9 | −45.874 | 1.650 | 48.038 | 1.00 | 47.62 | O |
| ATOM | 5 | CB | SER | A | 9 | −47.019 | −0.281 | 49.939 | 1.00 | 47.64 | C |
| ATOM | 6 | OG | SER | A | 9 | −48.326 | 0.166 | 50.264 | 1.00 | 47.56 | O |
| ATOM | 7 | N | LEU | A | 10 | −45.981 | 3.141 | 49.731 | 1.00 | 47.59 | N |
| ATOM | 8 | CA | LEU | A | 10 | −45.972 | 4.330 | 48.877 | 1.00 | 47.49 | C |
| ATOM | 9 | C | LEU | A | 10 | −44.717 | 5.189 | 49.123 | 1.00 | 47.36 | C |
| ATOM | 10 | O | LEU | A | 10 | −44.504 | 6.206 | 48.457 | 1.00 | 47.36 | O |
| ATOM | 11 | CB | LEU | A | 10 | −47.266 | 5.138 | 49.096 | 1.00 | 47.59 | C |
| ATOM | 12 | CG | LEU | A | 10 | −47.760 | 6.203 | 48.101 | 1.00 | 47.69 | C |
| ATOM | 13 | CD1 | LEU | A | 10 | −47.699 | 5.730 | 46.644 | 1.00 | 47.65 | C |
| ATOM | 14 | CD2 | LEU | A | 10 | −49.175 | 6.666 | 48.467 | 1.00 | 47.37 | C |
| ATOM | 15 | N | TYR | A | 11 | −43.885 | 4.754 | 50.070 | 1.00 | 47.15 | N |
| ATOM | 16 | CA | TYR | A | 11 | −42.650 | 5.457 | 50.434 | 1.00 | 46.82 | C |
| ATOM | 17 | C | TYR | A | 11 | −41.346 | 4.822 | 49.904 | 1.00 | 46.58 | C |
| ATOM | 18 | O | TYR | A | 11 | −40.357 | 5.540 | 49.730 | 1.00 | 46.57 | O |
| ATOM | 19 | CB | TYR | A | 11 | −42.559 | 5.624 | 51.959 | 1.00 | 46.84 | C |
| ATOM | 20 | CG | TYR | A | 11 | −41.551 | 4.711 | 52.635 | 1.00 | 46.86 | C |
| ATOM | 21 | CD1 | TYR | A | 11 | −40.240 | 5.137 | 52.856 | 1.00 | 46.91 | C |
| ATOM | 22 | CD2 | TYR | A | 11 | −41.906 | 3.427 | 53.053 | 1.00 | 46.86 | C |
| ATOM | 23 | CE1 | TYR | A | 11 | −39.306 | 4.311 | 53.467 | 1.00 | 46.85 | C |
| ATOM | 24 | CE2 | TYR | A | 11 | −40.977 | 2.591 | 53.670 | 1.00 | 46.86 | C |
| ATOM | 25 | CZ | TYR | A | 11 | −39.679 | 3.043 | 53.874 | 1.00 | 46.86 | C |
| ATOM | 26 | OH | TYR | A | 11 | −38.749 | 2.234 | 54.483 | 1.00 | 46.81 | O |
| ATOM | 27 | N | PRO | A | 12 | −41.326 | 3.483 | 49.667 | 1.00 | 46.30 | N |
| ATOM | 28 | CA | PRO | A | 12 | −40.064 | 2.836 | 49.271 | 1.00 | 45.95 | C |
| ATOM | 29 | C | PRO | A | 12 | −39.572 | 3.267 | 47.891 | 1.00 | 45.58 | C |
| ATOM | 30 | O | PRO | A | 12 | −38.525 | 2.801 | 47.438 | 1.00 | 45.64 | O |
| ATOM | 31 | CB | PRO | A | 12 | −40.417 | 1.338 | 49.259 | 1.00 | 45.96 | C |
| ATOM | 32 | CG | PRO | A | 12 | −41.704 | 1.223 | 50.011 | 1.00 | 46.11 | C |
| ATOM | 33 | CD | PRO | A | 12 | −42.422 | 2.498 | 49.733 | 1.00 | 46.27 | C |
| ATOM | 34 | N | ILE | A | 13 | −40.332 | 4.146 | 47.237 | 1.00 | 45.06 | N |
| ATOM | 35 | CA | ILE | A | 13 | −39.945 | 4.734 | 45.957 | 1.00 | 44.56 | C |
| ATOM | 36 | C | ILE | A | 13 | −38.875 | 5.804 | 46.189 | 1.00 | 44.07 | C |
| ATOM | 37 | O | ILE | A | 13 | −37.941 | 5.936 | 45.401 | 1.00 | 44.14 | O |
| ATOM | 38 | CB | ILE | A | 13 | −41.167 | 5.333 | 45.195 | 1.00 | 44.63 | C |
| ATOM | 39 | CG1 | ILE | A | 13 | −42.257 | 4.277 | 44.979 | 1.00 | 45.03 | C |
| ATOM | 40 | CG2 | ILE | A | 13 | −40.754 | 5.899 | 43.841 | 1.00 | 44.67 | C |
| ATOM | 41 | CD1 | ILE | A | 13 | −43.386 | 4.305 | 46.000 | 1.00 | 45.40 | C |
| ATOM | 42 | N | ALA | A | 14 | −39.016 | 6.550 | 47.282 | 1.00 | 43.47 | N |
| ATOM | 43 | CA | ALA | A | 14 | −38.036 | 7.553 | 47.690 | 1.00 | 42.87 | C |
| ATOM | 44 | C | ALA | A | 14 | −36.691 | 6.905 | 47.991 | 1.00 | 42.45 | C |
| ATOM | 45 | O | ALA | A | 14 | −35.638 | 7.461 | 47.678 | 1.00 | 42.23 | O |
| ATOM | 46 | CB | ALA | A | 14 | −38.542 | 8.311 | 48.909 | 1.00 | 42.89 | C |
| ATOM | 47 | N | VAL | A | 15 | −36.749 | 5.726 | 48.606 | 1.00 | 42.13 | N |
| ATOM | 48 | CA | VAL | A | 15 | −35.566 | 4.947 | 48.942 | 1.00 | 41.94 | C |
| ATOM | 49 | C | VAL | A | 15 | −34.948 | 4.406 | 47.661 | 1.00 | 41.87 | C |
| ATOM | 50 | O | VAL | A | 15 | −33.757 | 4.598 | 47.412 | 1.00 | 41.99 | O |
| ATOM | 51 | CB | VAL | A | 15 | −35.904 | 3.795 | 49.928 | 1.00 | 41.96 | C |
| ATOM | 52 | CG1 | VAL | A | 15 | −34.704 | 2.869 | 50.134 | 1.00 | 41.76 | C |
| ATOM | 53 | CG2 | VAL | A | 15 | −36.381 | 4.361 | 51.263 | 1.00 | 41.93 | C |
| ATOM | 54 | N | LEU | A | 16 | −35.772 | 3.751 | 46.846 | 1.00 | 41.67 | N |
| ATOM | 55 | CA | LEU | A | 16 | −35.347 | 3.240 | 45.546 | 1.00 | 41.51 | C |
| ATOM | 56 | C | LEU | A | 16 | −34.710 | 4.328 | 44.680 | 1.00 | 41.39 | C |
| ATOM | 57 | O | LEU | A | 16 | −33.677 | 4.095 | 44.055 | 1.00 | 41.17 | O |
| ATOM | 58 | CB | LEU | A | 16 | −36.531 | 2.600 | 44.811 | 1.00 | 41.56 | C |
| ATOM | 59 | CG | LEU | A | 16 | −36.296 | 1.983 | 43.430 | 1.00 | 41.34 | C |
| ATOM | 60 | CD1 | LEU | A | 16 | −35.449 | 0.716 | 43.507 | 1.00 | 41.09 | C |
| ATOM | 61 | CD2 | LEU | A | 16 | −37.629 | 1.703 | 42.768 | 1.00 | 41.42 | C |
| ATOM | 62 | N | ILE | A | 17 | −35.328 | 5.507 | 44.657 | 1.00 | 41.45 | N |
| ATOM | 63 | CA | ILE | A | 17 | −34.820 | 6.635 | 43.873 | 1.00 | 41.72 | C |
| ATOM | 64 | C | ILE | A | 17 | −33.444 | 7.084 | 44.361 | 1.00 | 41.80 | C |
| ATOM | 65 | O | ILE | A | 17 | −32.501 | 7.158 | 43.573 | 1.00 | 41.96 | O |
| ATOM | 66 | CB | ILE | A | 17 | −35.841 | 7.812 | 43.790 | 1.00 | 41.67 | C |
| ATOM | 67 | CG1 | ILE | A | 17 | −36.803 | 7.574 | 42.622 | 1.00 | 41.62 | C |
| ATOM | 68 | CG2 | ILE | A | 17 | −35.135 | 9.167 | 43.628 | 1.00 | 41.79 | C |
| ATOM | 69 | CD1 | ILE | A | 17 | −37.916 | 8.599 | 42.491 | 1.00 | 41.76 | C |
| ATOM | 70 | N | ASP | A | 18 | −33.330 | 7.352 | 45.658 | 1.00 | 41.89 | N |
| ATOM | 71 | CA | ASP | A | 18 | −32.080 | 7.819 | 46.247 | 1.00 | 41.98 | C |
| ATOM | 72 | C | ASP | A | 18 | −30.880 | 7.019 | 45.733 | 1.00 | 42.20 | C |
| ATOM | 73 | O | ASP | A | 18 | −29.863 | 7.586 | 45.323 | 1.00 | 42.19 | O |
| ATOM | 74 | CB | ASP | A | 18 | −32.162 | 7.724 | 47.768 | 1.00 | 41.76 | C |
| ATOM | 75 | CG | ASP | A | 18 | −31.061 | 8.486 | 48.450 | 1.00 | 41.56 | C |
| ATOM | 76 | OD1 | ASP | A | 18 | −31.016 | 9.723 | 48.297 | 1.00 | 41.43 | O |
| ATOM | 77 | OD2 | ASP | A | 18 | −30.242 | 7.849 | 49.139 | 1.00 | 41.59 | O |
| ATOM | 78 | N | GLU | A | 19 | −31.036 | 5.698 | 45.737 | 1.00 | 42.46 | N |
| ATOM | 79 | CA | GLU | A | 19 | −29.989 | 4.759 | 45.349 | 1.00 | 42.78 | C |
| ATOM | 80 | C | GLU | A | 19 | −29.751 | 4.724 | 43.838 | 1.00 | 42.75 | C |
| ATOM | 81 | O | GLU | A | 19 | −28.648 | 4.408 | 43.387 | 1.00 | 42.72 | O |
| ATOM | 82 | CB | GLU | A | 19 | −30.338 | 3.357 | 45.859 | 1.00 | 42.77 | C |
| ATOM | 83 | CG | GLU | A | 19 | −30.641 | 3.293 | 47.361 | 1.00 | 43.07 | C |
| ATOM | 84 | CD | GLU | A | 19 | −31.318 | 1.992 | 47.788 | 1.00 | 43.27 | C |
| ATOM | 85 | OE1 | GLU | A | 19 | −31.605 | 1.138 | 46.914 | 1.00 | 43.49 | O |
| ATOM | 86 | OE2 | GLU | A | 19 | −31.567 | 1.829 | 49.008 | 1.00 | 43.74 | O |
| ATOM | 87 | N | LEU | A | 20 | −30.785 | 5.045 | 43.066 | 1.00 | 42.85 | N |
| ATOM | 88 | CA | LEU | A | 20 | −30.680 | 5.065 | 41.609 | 1.00 | 43.10 | C |
| ATOM | 89 | C | LEU | A | 20 | −29.889 | 6.264 | 41.094 | 1.00 | 43.32 | C |
| ATOM | 90 | O | LEU | A | 20 | −29.451 | 6.274 | 39.939 | 1.00 | 43.42 | O |
| ATOM | 91 | CB | LEU | A | 20 | −32.065 | 5.026 | 40.953 | 1.00 | 43.04 | C |
| ATOM | 92 | CG | LEU | A | 20 | −32.864 | 3.717 | 41.001 | 1.00 | 43.12 | C |
| ATOM | 93 | CD1 | LEU | A | 20 | −34.139 | 3.858 | 40.180 | 1.00 | 42.96 | C |
| ATOM | 94 | CD2 | LEU | A | 20 | −32.047 | 2.511 | 40.532 | 1.00 | 42.48 | C |
| ATOM | 95 | N | ARG | A | 21 | −29.716 | 7.271 | 41.949 | 1.00 | 43.47 | N |
| ATOM | 96 | CA | ARG | A | 21 | −28.931 | 8.458 | 41.603 | 1.00 | 43.68 | C |
| ATOM | 97 | C | ARG | A | 21 | −27.649 | 8.616 | 42.437 | 1.00 | 43.88 | C |
| ATOM | 98 | O | ARG | A | 21 | −26.953 | 9.630 | 42.325 | 1.00 | 43.86 | O |
| ATOM | 99 | CB | ARG | A | 21 | −29.788 | 9.733 | 41.651 | 1.00 | 43.67 | C |
| ATOM | 100 | CG | ARG | A | 21 | −31.247 | 9.547 | 42.075 | 1.00 | 43.58 | C |
| ATOM | 101 | CD | ARG | A | 21 | −31.798 | 10.768 | 42.814 | 1.00 | 43.56 | C |
| ATOM | 102 | NE | ARG | A | 21 | −31.203 | 12.020 | 42.344 | 1.00 | 43.36 | N |
| ATOM | 103 | CZ | ARG | A | 21 | −31.419 | 13.216 | 42.885 | 1.00 | 43.07 | C |
| ATOM | 104 | NH1 | ARG | A | 21 | −32.234 | 13.351 | 43.923 | 1.00 | 43.06 | N |
| ATOM | 105 | NH2 | ARG | A | 21 | −30.815 | 14.284 | 42.381 | 1.00 | 43.02 | N |
| ATOM | 106 | N | ASN | A | 22 | −27.336 | 7.600 | 43.243 | 1.00 | 44.12 | N |
| ATOM | 107 | CA | ASN | A | 22 | −26.149 | 7.590 | 44.105 | 1.00 | 44.37 | C |
| ATOM | 108 | C | ASN | A | 22 | −24.823 | 7.653 | 43.332 | 1.00 | 44.74 | C |
| ATOM | 109 | O | ASN | A | 22 | −24.787 | 7.409 | 42.123 | 1.00 | 44.84 | O |
| ATOM | 110 | CB | ASN | A | 22 | −26.175 | 6.358 | 45.021 | 1.00 | 44.28 | C |
| ATOM | 111 | CG | ASN | A | 22 | −25.171 | 6.447 | 46.170 | 1.00 | 44.07 | C |
| ATOM | 112 | OD1 | ASN | A | 22 | −24.491 | 5.472 | 46.489 | 1.00 | 43.27 | O |
| ATOM | 113 | ND2 | ASN | A | 22 | −25.075 | 7.621 | 46.790 | 1.00 | 44.09 | N |
| ATOM | 114 | N | GLU | A | 23 | −23.742 | 7.982 | 44.042 | 1.00 | 45.08 | N |
| ATOM | 115 | CA | GLU | A | 23 | −22.399 | 8.087 | 43.455 | 1.00 | 45.35 | C |
| ATOM | 116 | C | GLU | A | 23 | −21.855 | 6.713 | 43.060 | 1.00 | 45.38 | C |
| ATOM | 117 | O | GLU | A | 23 | −21.403 | 6.518 | 41.929 | 1.00 | 45.32 | O |
| ATOM | 118 | CB | GLU | A | 23 | −21.416 | 8.776 | 44.423 | 1.00 | 45.43 | C |
| ATOM | 119 | CG | GLU | A | 23 | −22.038 | 9.731 | 45.463 | 1.00 | 45.80 | C |
| ATOM | 120 | CD | GLU | A | 23 | −22.931 | 10.803 | 44.849 | 1.00 | 46.34 | C |
| ATOM | 121 | OE1 | GLU | A | 23 | −22.452 | 11.572 | 43.988 | 1.00 | 46.26 | O |
| ATOM | 122 | OE2 | GLU | A | 23 | −24.119 | 10.873 | 45.236 | 1.00 | 46.62 | O |
| ATOM | 123 | N | ASP | A | 24 | −21.914 | 5.769 | 43.999 | 1.00 | 45.55 | N |
| ATOM | 124 | CA | ASP | A | 24 | −21.401 | 4.414 | 43.794 | 1.00 | 45.78 | C |
| ATOM | 125 | C | ASP | A | 24 | −22.184 | 3.639 | 42.737 | 1.00 | 45.78 | C |
| ATOM | 126 | O | ASP | A | 24 | −23.407 | 3.472 | 42.843 | 1.00 | 45.75 | O |
| ATOM | 127 | CB | ASP | A | 24 | −21.378 | 3.633 | 45.114 | 1.00 | 45.86 | C |
| ATOM | 128 | CG | ASP | A | 24 | −20.297 | 4.121 | 46.067 | 1.00 | 46.31 | C |
| ATOM | 129 | OD1 | ASP | A | 24 | −19.099 | 4.039 | 45.714 | 1.00 | 46.60 | O |
| ATOM | 130 | OD2 | ASP | A | 24 | −20.646 | 4.575 | 47.178 | 1.00 | 46.73 | O |
| ATOM | 131 | N | VAL | A | 25 | −21.452 | 3.173 | 41.727 | 1.00 | 45.72 | N |
| ATOM | 132 | CA | VAL | A | 25 | −21.999 | 2.390 | 40.620 | 1.00 | 45.73 | C |
| ATOM | 133 | C | VAL | A | 25 | −22.726 | 1.135 | 41.127 | 1.00 | 45.87 | C |
| ATOM | 134 | O | VAL | A | 25 | −23.742 | 0.723 | 40.555 | 1.00 | 45.82 | O |
| ATOM | 135 | CB | VAL | A | 25 | −20.877 | 1.963 | 39.618 | 1.00 | 45.74 | C |
| ATOM | 136 | CG1 | VAL | A | 25 | −21.466 | 1.606 | 38.258 | 1.00 | 45.49 | C |
| ATOM | 137 | CG2 | VAL | A | 25 | −19.827 | 3.061 | 39.460 | 1.00 | 45.64 | C |
| ATOM | 138 | N | GLN | A | 26 | −22.207 | 0.550 | 42.209 | 1.00 | 45.92 | N |
| ATOM | 139 | CA | GLN | A | 26 | −22.716 | −0.717 | 42.747 | 1.00 | 45.88 | C |
| ATOM | 140 | C | GLN | A | 26 | −24.166 | −0.655 | 43.235 | 1.00 | 45.79 | C |
| ATOM | 141 | O | GLN | A | 26 | −24.956 | −1.563 | 42.963 | 1.00 | 45.83 | O |
| ATOM | 142 | CB | GLN | A | 26 | −21.792 | −1.261 | 43.846 | 1.00 | 45.87 | C |
| ATOM | 143 | CG | GLN | A | 26 | −20.402 | −1.669 | 43.355 | 1.00 | 46.28 | C |
| ATOM | 144 | CD | GLN | A | 26 | −20.439 | −2.522 | 42.087 | 1.00 | 47.09 | C |
| ATOM | 145 | OE1 | GLN | A | 26 | −21.183 | −3.504 | 42.001 | 1.00 | 47.28 | O |
| ATOM | 146 | NE2 | GLN | A | 26 | −19.632 | −2.147 | 41.097 | 1.00 | 46.85 | N |
| ATOM | 147 | N | LEU | A | 27 | −24.512 | 0.412 | 43.947 | 1.00 | 45.54 | N |
| ATOM | 148 | CA | LEU | A | 27 | −25.882 | 0.595 | 44.401 | 1.00 | 45.43 | C |
| ATOM | 149 | C | LEU | A | 27 | −26.832 | 0.767 | 43.226 | 1.00 | 45.31 | C |
| ATOM | 150 | O | LEU | A | 27 | −27.861 | 0.095 | 43.156 | 1.00 | 45.15 | O |
| ATOM | 151 | CB | LEU | A | 27 | −25.980 | 1.784 | 45.354 | 1.00 | 45.59 | C |
| ATOM | 152 | CG | LEU | A | 27 | −25.955 | 1.419 | 46.837 | 1.00 | 45.49 | C |
| ATOM | 153 | CD1 | LEU | A | 27 | −25.091 | 2.396 | 47.616 | 1.00 | 45.68 | C |
| ATOM | 154 | CD2 | LEU | A | 27 | −27.374 | 1.354 | 47.392 | 1.00 | 45.31 | C |
| ATOM | 155 | N | ARG | A | 28 | −26.462 | 1.660 | 42.307 | 1.00 | 45.31 | N |
| ATOM | 156 | CA | ARG | A | 28 | −27.217 | 1.915 | 41.077 | 1.00 | 45.28 | C |
| ATOM | 157 | C | ARG | A | 28 | −27.597 | 0.639 | 40.317 | 1.00 | 45.30 | C |
| ATOM | 158 | O | ARG | A | 28 | −28.774 | 0.404 | 40.039 | 1.00 | 45.29 | O |
| ATOM | 159 | CB | ARG | A | 28 | −26.420 | 2.827 | 40.146 | 1.00 | 45.20 | C |
| ATOM | 160 | CG | ARG | A | 28 | −26.428 | 4.296 | 40.500 | 1.00 | 45.27 | C |
| ATOM | 161 | CD | ARG | A | 28 | −25.446 | 5.015 | 39.601 | 1.00 | 46.27 | C |
| ATOM | 162 | NE | ARG | A | 28 | −25.573 | 6.468 | 39.649 | 1.00 | 46.87 | N |
| ATOM | 163 | CZ | ARG | A | 28 | −24.823 | 7.309 | 38.938 | 1.00 | 46.99 | C |
| ATOM | 164 | NH1 | ARG | A | 28 | −23.884 | 6.842 | 38.117 | 1.00 | 46.36 | N |
| ATOM | 165 | NH2 | ARG | A | 28 | −25.010 | 8.621 | 39.049 | 1.00 | 46.47 | N |
| ATOM | 166 | N | LEU | A | 29 | −26.596 | −0.177 | 39.994 | 1.00 | 45.30 | N |
| ATOM | 167 | CA | LEU | A | 29 | −26.786 | −1.352 | 39.140 | 1.00 | 45.39 | C |
| ATOM | 168 | C | LEU | A | 29 | −27.727 | −2.400 | 39.725 | 1.00 | 45.52 | C |
| ATOM | 169 | O | LEU | A | 29 | −28.615 | −2.905 | 39.031 | 1.00 | 45.48 | O |
| ATOM | 170 | CB | LEU | A | 29 | −25.433 | −1.992 | 38.799 | 1.00 | 45.38 | C |
| ATOM | 171 | CG | LEU | A | 29 | −24.703 | −1.487 | 37.552 | 1.00 | 45.04 | C |
| ATOM | 172 | CD1 | LEU | A | 29 | −23.213 | −1.664 | 37.717 | 1.00 | 44.70 | C |
| ATOM | 173 | CD2 | LEU | A | 29 | −25.196 | −2.200 | 36.303 | 1.00 | 44.78 | C |
| ATOM | 174 | N | ASN | A | 30 | −27.523 | −2.727 | 40.997 | 1.00 | 45.69 | N |
| ATOM | 175 | CA | ASN | A | 30 | −28.294 | −3.776 | 41.658 | 1.00 | 45.81 | C |
| ATOM | 176 | C | ASN | A | 30 | −29.726 | −3.356 | 41.965 | 1.00 | 45.67 | C |
| ATOM | 177 | O | ASN | A | 30 | −30.659 | −4.139 | 41.779 | 1.00 | 45.75 | O |
| ATOM | 178 | CB | ASN | A | 30 | −27.567 | −4.259 | 42.914 | 1.00 | 45.88 | C |
| ATOM | 179 | CG | ASN | A | 30 | −26.183 | −4.808 | 42.603 | 1.00 | 46.46 | C |
| ATOM | 180 | OD1 | ASN | A | 30 | −26.018 | −5.642 | 41.705 | 1.00 | 46.95 | O |
| ATOM | 181 | ND2 | ASN | A | 30 | −25.178 | −4.333 | 43.333 | 1.00 | 46.65 | N |
| ATOM | 182 | N | SER | A | 31 | −29.900 | −2.113 | 42.409 | 1.00 | 45.40 | N |
| ATOM | 183 | CA | SER | A | 31 | −31.234 | −1.564 | 42.627 | 1.00 | 45.26 | C |
| ATOM | 184 | C | SER | A | 31 | −32.047 | −1.454 | 41.330 | 1.00 | 45.18 | C |
| ATOM | 185 | O | SER | A | 31 | −33.277 | −1.449 | 41.376 | 1.00 | 45.22 | O |
| ATOM | 186 | CB | SER | A | 31 | −31.157 | −0.213 | 43.335 | 1.00 | 45.21 | C |
| ATOM | 187 | OG | SER | A | 31 | −30.207 | 0.627 | 42.713 | 1.00 | 45.04 | O |
| ATOM | 188 | N | ILE | A | 32 | −31.359 | −1.367 | 40.187 | 1.00 | 45.04 | N |
| ATOM | 189 | CA | ILE | A | 32 | −32.010 | −1.402 | 38.868 | 1.00 | 44.86 | C |
| ATOM | 190 | C | ILE | A | 32 | −32.662 | −2.768 | 38.622 | 1.00 | 44.99 | C |
| ATOM | 191 | O | ILE | A | 32 | −33.803 | −2.844 | 38.160 | 1.00 | 45.02 | O |
| ATOM | 192 | CB | ILE | A | 32 | −31.031 | −1.035 | 37.710 | 1.00 | 44.84 | C |
| ATOM | 193 | CG1 | ILE | A | 32 | −30.885 | 0.486 | 37.589 | 1.00 | 44.67 | C |
| ATOM | 194 | CG2 | ILE | A | 32 | −31.510 | −1.620 | 36.372 | 1.00 | 44.70 | C |
| ATOM | 195 | CD1 | ILE | A | 32 | −29.649 | 0.951 | 36.826 | 1.00 | 44.64 | C |
| ATOM | 196 | N | LYS | A | 33 | −31.935 | −3.836 | 38.946 | 1.00 | 45.08 | N |
| ATOM | 197 | CA | LYS | A | 33 | −32.456 | −5.200 | 38.863 | 1.00 | 45.02 | C |
| ATOM | 198 | C | LYS | A | 33 | −33.314 | −5.488 | 40.098 | 1.00 | 45.03 | C |
| ATOM | 199 | O | LYS | A | 33 | −33.096 | −6.461 | 40.823 | 1.00 | 45.05 | O |
| ATOM | 200 | CB | LYS | A | 33 | −31.306 | −6.200 | 38.719 | 1.00 | 45.04 | C |
| ATOM | 201 | CG | LYS | A | 33 | −30.487 | −6.022 | 37.440 | 1.00 | 45.11 | C |
| ATOM | 202 | CD | LYS | A | 33 | −29.217 | −6.863 | 37.464 | 1.00 | 45.12 | C |
| ATOM | 203 | CE | LYS | A | 33 | −28.338 | −6.582 | 36.250 | 1.00 | 44.89 | C |
| ATOM | 204 | NZ | LYS | A | 33 | −27.067 | −7.359 | 36.303 | 1.00 | 44.53 | N |
| ATOM | 205 | N | LYS | A | 34 | −34.298 | −4.615 | 40.306 | 1.00 | 45.10 | N |
| ATOM | 206 | CA | LYS | A | 34 | −35.165 | −4.591 | 41.480 | 1.00 | 45.12 | C |
| ATOM | 207 | C | LYS | A | 34 | −36.431 | −3.812 | 41.092 | 1.00 | 45.19 | C |
| ATOM | 208 | O | LYS | A | 34 | −37.455 | −3.865 | 41.784 | 1.00 | 45.22 | O |
| ATOM | 209 | CB | LYS | A | 34 | −34.429 | −3.905 | 42.638 | 1.00 | 45.11 | C |
| ATOM | 210 | CG | LYS | A | 34 | −35.131 | −3.891 | 43.993 | 1.00 | 45.20 | C |
| ATOM | 211 | CD | LYS | A | 34 | −34.175 | −3.397 | 45.086 | 1.00 | 45.18 | C |
| ATOM | 212 | CE | LYS | A | 34 | −34.895 | −3.150 | 46.406 | 1.00 | 45.28 | C |
| ATOM | 213 | NZ | LYS | A | 34 | −33.951 | −2.862 | 47.523 | 1.00 | 44.82 | N |
| ATOM | 214 | N | LEU | A | 35 | −36.340 | −3.104 | 39.964 | 1.00 | 45.08 | N |
| ATOM | 215 | CA | LEU | A | 35 | −37.422 | −2.274 | 39.435 | 1.00 | 45.06 | C |
| ATOM | 216 | C | LEU | A | 35 | −38.635 | −3.065 | 38.943 | 1.00 | 45.10 | C |
| ATOM | 217 | O | LEU | A | 35 | −39.768 | −2.584 | 39.029 | 1.00 | 45.16 | O |
| ATOM | 218 | CB | LEU | A | 35 | −36.898 | −1.385 | 38.301 | 1.00 | 45.08 | C |
| ATOM | 219 | CG | LEU | A | 35 | −36.491 | 0.065 | 38.595 | 1.00 | 45.06 | C |
| ATOM | 220 | CD1 | LEU | A | 35 | −35.435 | 0.166 | 39.685 | 1.00 | 45.05 | C |
| ATOM | 221 | CD2 | LEU | A | 35 | −36.005 | 0.741 | 37.321 | 1.00 | 45.02 | C |
| ATOM | 222 | N | SER | A | 36 | −38.395 | −4.267 | 38.421 | 1.00 | 44.98 | N |
| ATOM | 223 | CA | SER | A | 36 | −39.471 | −5.119 | 37.912 | 1.00 | 44.89 | C |
| ATOM | 224 | C | SER | A | 36 | −40.520 | −5.424 | 38.984 | 1.00 | 44.89 | C |
| ATOM | 225 | O | SER | A | 36 | −41.720 | −5.282 | 38.744 | 1.00 | 44.88 | O |
| ATOM | 226 | CB | SER | A | 36 | −38.903 | −6.417 | 37.335 | 1.00 | 44.87 | C |
| ATOM | 227 | OG | SER | A | 36 | −38.402 | −7.255 | 38.361 | 1.00 | 44.84 | O |
| ATOM | 228 | N | THR | A | 37 | −40.056 | −5.831 | 40.164 | 1.00 | 44.85 | N |
| ATOM | 229 | CA | THR | A | 37 | −40.933 | −6.162 | 41.287 | 1.00 | 44.89 | C |
| ATOM | 230 | C | THR | A | 37 | −41.637 | −4.919 | 41.828 | 1.00 | 44.87 | C |
| ATOM | 231 | O | THR | A | 37 | −42.774 | −4.993 | 42.303 | 1.00 | 44.79 | O |
| ATOM | 232 | CB | THR | A | 37 | −40.145 | −6.833 | 42.430 | 1.00 | 44.96 | C |
| ATOM | 233 | OG1 | THR | A | 37 | −39.204 | −7.765 | 41.882 | 1.00 | 45.07 | O |
| ATOM | 234 | CG2 | THR | A | 37 | −41.086 | −7.558 | 43.399 | 1.00 | 44.93 | C |
| ATOM | 235 | N | ILE | A | 38 | −40.950 | −3.783 | 41.748 | 1.00 | 44.87 | N |
| ATOM | 236 | CA | ILE | A | 38 | −41.492 | −2.506 | 42.204 | 1.00 | 44.94 | C |
| ATOM | 237 | C | ILE | A | 38 | −42.472 | −1.915 | 41.183 | 1.00 | 44.87 | C |
| ATOM | 238 | O | ILE | A | 38 | −43.362 | −1.139 | 41.539 | 1.00 | 44.86 | O |
| ATOM | 239 | CB | ILE | A | 38 | −40.351 | −1.532 | 42.589 | 1.00 | 45.01 | C |
| ATOM | 240 | CG1 | ILE | A | 38 | −39.731 | −1.987 | 43.914 | 1.00 | 45.29 | C |
| ATOM | 241 | CG2 | ILE | A | 38 | −40.855 | −0.095 | 42.708 | 1.00 | 44.98 | C |
| ATOM | 242 | CD1 | ILE | A | 38 | −38.264 | −1.654 | 44.080 | 1.00 | 45.94 | C |
| ATOM | 243 | N | ALA | A | 39 | −42.314 | −2.302 | 39.919 | 1.00 | 44.86 | N |
| ATOM | 244 | CA | ALA | A | 39 | −43.283 | −1.959 | 38.880 | 1.00 | 44.81 | C |
| ATOM | 245 | C | ALA | A | 39 | −44.604 | −2.697 | 39.116 | 1.00 | 44.80 | C |
| ATOM | 246 | O | ALA | A | 39 | −45.678 | −2.095 | 39.044 | 1.00 | 44.84 | O |
| ATOM | 247 | CB | ALA | A | 39 | −42.723 | −2.276 | 37.499 | 1.00 | 44.78 | C |
| ATOM | 248 | N | LEU | A | 40 | −44.513 | −3.992 | 39.419 | 1.00 | 44.74 | N |
| ATOM | 249 | CA | LEU | A | 40 | −45.686 | −4.818 | 39.717 | 1.00 | 44.66 | C |
| ATOM | 250 | C | LEU | A | 40 | −46.303 | −4.514 | 41.082 | 1.00 | 44.68 | C |
| ATOM | 251 | O | LEU | A | 40 | −47.380 | −5.020 | 41.399 | 1.00 | 44.66 | O |
| ATOM | 252 | CB | LEU | A | 40 | −45.344 | −6.312 | 39.621 | 1.00 | 44.63 | C |
| ATOM | 253 | CG | LEU | A | 40 | −45.308 | −6.979 | 38.241 | 1.00 | 44.53 | C |
| ATOM | 254 | CD1 | LEU | A | 40 | −44.633 | −8.339 | 38.323 | 1.00 | 44.45 | C |
| ATOM | 255 | CD2 | LEU | A | 40 | −46.705 | −7.111 | 37.644 | 1.00 | 44.44 | C |
| ATOM | 256 | N | ALA | A | 41 | −45.617 | −3.699 | 41.885 | 1.00 | 44.75 | N |
| ATOM | 257 | CA | ALA | A | 41 | −46.132 | −3.273 | 43.188 | 1.00 | 44.81 | C |
| ATOM | 258 | C | ALA | A | 41 | −47.414 | −2.464 | 43.010 | 1.00 | 44.91 | C |
| ATOM | 259 | O | ALA | A | 41 | −48.467 | −2.839 | 43.536 | 1.00 | 44.94 | O |
| ATOM | 260 | CB | ALA | A | 41 | −45.082 | −2.475 | 43.954 | 1.00 | 44.76 | C |
| ATOM | 261 | N | LEU | A | 42 | −47.315 | −1.364 | 42.261 | 1.00 | 45.00 | N |
| ATOM | 262 | CA | LEU | A | 42 | −48.475 | −0.562 | 41.850 | 1.00 | 45.08 | C |
| ATOM | 263 | C | LEU | A | 42 | −48.042 | 0.440 | 40.777 | 1.00 | 45.12 | C |
| ATOM | 264 | O | LEU | A | 42 | −47.798 | 1.612 | 41.073 | 1.00 | 45.11 | O |
| ATOM | 265 | CB | LEU | A | 42 | −49.122 | 0.153 | 43.054 | 1.00 | 45.11 | C |
| ATOM | 266 | CG | LEU | A | 42 | −50.622 | 0.484 | 43.003 | 1.00 | 45.01 | C |
| ATOM | 267 | CD1 | LEU | A | 42 | −51.488 | −0.763 | 43.191 | 1.00 | 44.75 | C |
| ATOM | 268 | CD2 | LEU | A | 42 | −50.972 | 1.532 | 44.050 | 1.00 | 44.97 | C |
| ATOM | 269 | N | GLY | A | 43 | −47.947 | −0.032 | 39.533 | 1.00 | 45.18 | N |
| ATOM | 270 | CA | GLY | A | 43 | −47.391 | 0.766 | 38.438 | 1.00 | 45.23 | C |
| ATOM | 271 | C | GLY | A | 43 | −48.265 | 0.942 | 37.211 | 1.00 | 45.29 | C |
| ATOM | 272 | O | GLY | A | 43 | −48.010 | 0.341 | 36.167 | 1.00 | 45.25 | O |
| ATOM | 273 | N | VAL | A | 44 | −49.297 | 1.772 | 37.344 | 1.00 | 45.40 | N |
| ATOM | 274 | CA | VAL | A | 44 | −50.144 | 2.166 | 36.215 | 1.00 | 45.40 | C |
| ATOM | 275 | C | VAL | A | 44 | −49.887 | 3.647 | 35.914 | 1.00 | 45.45 | C |
| ATOM | 276 | O | VAL | A | 44 | −49.630 | 4.017 | 34.768 | 1.00 | 45.50 | O |
| ATOM | 277 | CB | VAL | A | 44 | −51.662 | 1.899 | 36.481 | 1.00 | 45.38 | C |
| ATOM | 278 | CG1 | VAL | A | 44 | −52.500 | 2.213 | 35.244 | 1.00 | 45.23 | C |
| ATOM | 279 | CG2 | VAL | A | 44 | −51.897 | 0.455 | 36.919 | 1.00 | 45.24 | C |
| ATOM | 280 | N | GLU | A | 45 | −49.953 | 4.478 | 36.954 | 1.00 | 45.47 | N |
| ATOM | 281 | CA | GLU | A | 45 | −49.625 | 5.904 | 36.864 | 1.00 | 45.49 | C |
| ATOM | 282 | C | GLU | A | 45 | −48.313 | 6.235 | 37.574 | 1.00 | 45.55 | C |
| ATOM | 283 | O | GLU | A | 45 | −47.720 | 7.289 | 37.334 | 1.00 | 45.58 | O |
| ATOM | 284 | CB | GLU | A | 45 | −50.749 | 6.760 | 37.457 | 1.00 | 45.44 | C |
| ATOM | 285 | CG | GLU | A | 45 | −51.594 | 7.502 | 36.430 | 1.00 | 45.45 | C |
| ATOM | 286 | CD | GLU | A | 45 | −52.507 | 8.562 | 37.050 | 1.00 | 45.44 | C |
| ATOM | 287 | OE1 | GLU | A | 45 | −53.035 | 8.339 | 38.162 | 1.00 | 45.26 | O |
| ATOM | 288 | OE2 | GLU | A | 45 | −52.704 | 9.623 | 36.418 | 1.00 | 45.12 | O |
| ATOM | 289 | N | ARG | A | 46 | −47.876 | 5.341 | 38.458 | 1.00 | 45.58 | N |
| ATOM | 290 | CA | ARG | A | 46 | −46.671 | 5.557 | 39.260 | 1.00 | 45.58 | C |
| ATOM | 291 | C | ARG | A | 46 | −45.408 | 5.554 | 38.405 | 1.00 | 45.42 | C |
| ATOM | 292 | O | ARG | A | 46 | −44.587 | 6.469 | 38.486 | 1.00 | 45.36 | O |
| ATOM | 293 | CB | ARG | A | 46 | −46.577 | 4.510 | 40.374 | 1.00 | 45.68 | C |
| ATOM | 294 | CG | ARG | A | 46 | −47.400 | 4.850 | 41.608 | 1.00 | 46.06 | C |
| ATOM | 295 | CD | ARG | A | 46 | −46.658 | 5.853 | 42.473 | 1.00 | 46.85 | C |
| ATOM | 296 | NE | ARG | A | 46 | −47.563 | 6.736 | 43.202 | 1.00 | 47.18 | N |
| ATOM | 297 | CZ | ARG | A | 46 | −47.205 | 7.905 | 43.723 | 1.00 | 47.25 | C |
| ATOM | 298 | NH1 | ARG | A | 46 | −45.956 | 8.342 | 43.594 | 1.00 | 47.19 | N |
| ATOM | 299 | NH2 | ARG | A | 46 | −48.098 | 8.643 | 44.370 | 1.00 | 47.22 | N |
| ATOM | 300 | N | LEU | A | 400 | −45.273 | 4.528 | 37.572 | 1.00 | 45.28 | N |
| ATOM | 301 | CA | LEU | A | 400 | −44.158 | 4.432 | 36.639 | 1.00 | 45.12 | C |
| ATOM | 302 | C | LEU | A | 400 | −44.125 | 5.613 | 35.670 | 1.00 | 45.01 | C |
| ATOM | 303 | O | LEU | A | 400 | −43.056 | 6.006 | 35.219 | 1.00 | 45.02 | O |
| ATOM | 304 | CB | LEU | A | 400 | −44.223 | 3.108 | 35.873 | 1.00 | 45.12 | C |
| ATOM | 305 | CG | LEU | A | 400 | −43.624 | 1.829 | 36.480 | 1.00 | 45.12 | C |
| ATOM | 306 | CD1 | LEU | A | 400 | −43.935 | 1.637 | 37.966 | 1.00 | 44.96 | C |
| ATOM | 307 | CD2 | LEU | A | 400 | −44.088 | 0.617 | 35.677 | 1.00 | 45.18 | C |
| ATOM | 308 | N | SER | A | 401 | −45.296 | 6.181 | 35.381 | 1.00 | 44.90 | N |
| ATOM | 309 | CA | SER | A | 401 | −45.448 | 7.292 | 34.435 | 1.00 | 44.83 | C |
| ATOM | 310 | C | SER | A | 401 | −44.541 | 8.491 | 34.737 | 1.00 | 44.80 | C |
| ATOM | 311 | O | SER | A | 401 | −43.793 | 8.935 | 33.862 | 1.00 | 44.70 | O |
| ATOM | 312 | CB | SER | A | 401 | −46.922 | 7.733 | 34.357 | 1.00 | 44.91 | C |
| ATOM | 313 | OG | SER | A | 401 | −47.158 | 8.658 | 33.304 | 1.00 | 44.80 | O |
| ATOM | 314 | N | GLN | A | 402 | −44.601 | 9.007 | 35.966 | 1.00 | 44.76 | N |
| ATOM | 315 | CA | GLN | A | 402 | −43.862 | 10.229 | 36.321 | 1.00 | 44.77 | C |
| ATOM | 316 | C | GLN | A | 402 | −43.057 | 10.082 | 37.614 | 1.00 | 44.68 | C |
| ATOM | 317 | O | GLN | A | 402 | −42.051 | 10.772 | 37.809 | 1.00 | 44.70 | O |
| ATOM | 318 | CB | GLN | A | 402 | −44.818 | 11.432 | 36.440 | 1.00 | 44.99 | C |
| ATOM | 319 | CG | GLN | A | 402 | −44.378 | 12.711 | 35.689 | 1.00 | 44.98 | C |
| ATOM | 320 | CD | GLN | A | 402 | −43.066 | 13.319 | 36.190 | 1.00 | 44.88 | C |
| ATOM | 321 | OE1 | GLN | A | 402 | −42.787 | 13.342 | 37.389 | 1.00 | 44.68 | O |
| ATOM | 322 | NE2 | GLN | A | 402 | −42.263 | 13.831 | 35.263 | 1.00 | 44.73 | N |
| ATOM | 323 | N | SER | A | 403 | −43.498 | 9.189 | 38.498 | 1.00 | 44.45 | N |
| ATOM | 324 | CA | SER | A | 403 | −42.827 | 9.008 | 39.788 | 1.00 | 44.18 | C |
| ATOM | 325 | C | SER | A | 403 | −41.538 | 8.187 | 39.702 | 1.00 | 43.96 | C |
| ATOM | 326 | O | SER | A | 403 | −40.779 | 8.145 | 40.670 | 1.00 | 44.13 | O |
| ATOM | 327 | CB | SER | A | 403 | −43.779 | 8.400 | 40.829 | 1.00 | 44.23 | C |
| ATOM | 328 | OG | SER | A | 403 | −44.755 | 9.337 | 41.256 | 1.00 | 44.03 | O |
| ATOM | 329 | N | LEU | A | 404 | −41.282 | 7.559 | 38.551 | 1.00 | 43.61 | N |
| ATOM | 330 | CA | LEU | A | 404 | −40.164 | 6.614 | 38.422 | 1.00 | 43.17 | C |
| ATOM | 331 | C | LEU | A | 404 | −39.509 | 6.558 | 37.035 | 1.00 | 42.91 | C |
| ATOM | 332 | O | LEU | A | 404 | −38.306 | 6.328 | 36.931 | 1.00 | 42.79 | O |
| ATOM | 333 | CB | LEU | A | 404 | −40.623 | 5.215 | 38.840 | 1.00 | 43.22 | C |
| ATOM | 334 | CG | LEU | A | 404 | −39.620 | 4.319 | 39.561 | 1.00 | 43.05 | C |
| ATOM | 335 | CD1 | LEU | A | 404 | −40.313 | 3.509 | 40.640 | 1.00 | 43.34 | C |
| ATOM | 336 | CD2 | LEU | A | 404 | −38.922 | 3.409 | 38.579 | 1.00 | 43.72 | C |
| ATOM | 337 | N | LEU | A | 405 | −40.293 | 6.757 | 35.976 | 1.00 | 42.61 | N |
| ATOM | 338 | CA | LEU | A | 405 | −39.747 | 6.729 | 34.610 | 1.00 | 42.28 | C |
| ATOM | 339 | C | LEU | A | 405 | −38.676 | 7.798 | 34.353 | 1.00 | 41.98 | C |
| ATOM | 340 | O | LEU | A | 405 | −37.659 | 7.492 | 33.735 | 1.00 | 41.98 | O |
| ATOM | 341 | CB | LEU | A | 405 | −40.853 | 6.785 | 33.543 | 1.00 | 42.16 | C |
| ATOM | 342 | CG | LEU | A | 405 | −40.474 | 6.614 | 32.065 | 1.00 | 42.34 | C |
| ATOM | 343 | CD1 | LEU | A | 405 | −39.823 | 5.263 | 31.784 | 1.00 | 42.39 | C |
| ATOM | 344 | CD2 | LEU | A | 405 | −41.689 | 6.804 | 31.179 | 1.00 | 42.20 | C |
| ATOM | 345 | N | PRO | A | 406 | −38.896 | 9.049 | 34.819 | 1.00 | 41.73 | N |
| ATOM | 346 | CA | PRO | A | 406 | −37.857 | 10.068 | 34.661 | 1.00 | 41.72 | C |
| ATOM | 347 | C | PRO | A | 406 | −36.473 | 9.606 | 35.127 | 1.00 | 41.74 | C |
| ATOM | 348 | O | PRO | A | 406 | −35.478 | 9.850 | 34.442 | 1.00 | 41.63 | O |
| ATOM | 349 | CB | PRO | A | 406 | −38.366 | 11.213 | 35.537 | 1.00 | 41.66 | C |
| ATOM | 350 | CG | PRO | A | 406 | −39.827 | 11.085 | 35.467 | 1.00 | 41.56 | C |
| ATOM | 351 | CD | PRO | A | 406 | −40.089 | 9.609 | 35.482 | 1.00 | 41.60 | C |
| ATOM | 352 | N | ALA | A | 407 | −36.420 | 8.932 | 36.272 | 1.00 | 41.81 | N |
| ATOM | 353 | CA | ALA | A | 407 | −35.170 | 8.392 | 36.790 | 1.00 | 41.89 | C |
| ATOM | 354 | C | ALA | A | 407 | −34.587 | 7.316 | 35.855 | 1.00 | 42.13 | C |
| ATOM | 355 | O | ALA | A | 407 | −33.365 | 7.219 | 35.692 | 1.00 | 41.95 | O |
| ATOM | 356 | CB | ALA | A | 407 | −35.373 | 7.853 | 38.200 | 1.00 | 41.73 | C |
| ATOM | 357 | N | ILE | A | 408 | −35.469 | 6.528 | 35.235 | 1.00 | 42.40 | N |
| ATOM | 358 | CA | ILE | A | 408 | −35.067 | 5.507 | 34.262 | 1.00 | 42.53 | C |
| ATOM | 359 | C | ILE | A | 408 | −34.496 | 6.172 | 33.007 | 1.00 | 42.89 | C |
| ATOM | 360 | O | ILE | A | 408 | −33.458 | 5.749 | 32.490 | 1.00 | 42.89 | O |
| ATOM | 361 | CB | ILE | A | 408 | −36.251 | 4.568 | 33.892 | 1.00 | 42.41 | C |
| ATOM | 362 | CG1 | ILE | A | 408 | −36.773 | 3.841 | 35.135 | 1.00 | 42.18 | C |
| ATOM | 363 | CG2 | ILE | A | 408 | −35.841 | 3.553 | 32.819 | 1.00 | 42.13 | C |
| ATOM | 364 | CD1 | ILE | A | 408 | −38.162 | 3.259 | 34.966 | 1.00 | 41.95 | C |
| ATOM | 365 | N | VAL | A | 409 | −35.171 | 7.225 | 32.544 | 1.00 | 43.37 | N |
| ATOM | 366 | CA | VAL | A | 409 | −34.773 | 7.976 | 31.345 | 1.00 | 43.73 | C |
| ATOM | 367 | C | VAL | A | 409 | −33.349 | 8.532 | 31.426 | 1.00 | 43.96 | C |
| ATOM | 368 | O | VAL | A | 409 | −32.588 | 8.436 | 30.460 | 1.00 | 43.94 | O |
| ATOM | 369 | CB | VAL | A | 409 | −35.783 | 9.104 | 31.028 | 1.00 | 43.73 | C |
| ATOM | 370 | CG1 | VAL | A | 409 | −35.193 | 10.138 | 30.059 | 1.00 | 43.73 | C |
| ATOM | 371 | CG2 | VAL | A | 409 | −37.069 | 8.508 | 30.469 | 1.00 | 43.92 | C |
| ATOM | 372 | N | GLU | A | 410 | −32.997 | 9.113 | 32.570 | 1.00 | 44.30 | N |
| ATOM | 373 | CA | GLU | A | 410 | −31.653 | 9.649 | 32.765 | 1.00 | 44.71 | C |
| ATOM | 374 | C | GLU | A | 410 | −30.633 | 8.518 | 32.856 | 1.00 | 44.76 | C |
| ATOM | 375 | O | GLU | A | 410 | −29.559 | 8.603 | 32.264 | 1.00 | 44.95 | O |
| ATOM | 376 | CB | GLU | A | 410 | −31.587 | 10.551 | 33.999 | 1.00 | 44.63 | C |
| ATOM | 377 | CG | GLU | A | 410 | −30.321 | 11.405 | 34.062 | 1.00 | 45.13 | C |
| ATOM | 378 | CD | GLU | A | 410 | −30.427 | 12.587 | 35.023 | 1.00 | 45.15 | C |
| ATOM | 379 | OE1 | GLU | A | 410 | −31.523 | 12.821 | 35.585 | 1.00 | 45.52 | O |
| ATOM | 380 | OE2 | GLU | A | 410 | −29.404 | 13.286 | 35.210 | 1.00 | 45.27 | O |
| ATOM | 381 | N | LEU | A | 411 | −30.986 | 7.455 | 33.579 | 1.00 | 44.92 | N |
| ATOM | 382 | CA | LEU | A | 411 | −30.122 | 6.273 | 33.708 | 1.00 | 44.98 | C |
| ATOM | 383 | C | LEU | A | 411 | −29.829 | 5.613 | 32.363 | 1.00 | 45.02 | C |
| ATOM | 384 | O | LEU | A | 411 | −28.856 | 4.867 | 32.227 | 1.00 | 44.98 | O |
| ATOM | 385 | CB | LEU | A | 411 | −30.737 | 5.244 | 34.663 | 1.00 | 44.93 | C |
| ATOM | 386 | CG | LEU | A | 411 | −30.696 | 5.489 | 36.171 | 1.00 | 44.86 | C |
| ATOM | 387 | CD1 | LEU | A | 411 | −31.653 | 4.528 | 36.856 | 1.00 | 45.46 | C |
| ATOM | 388 | CD2 | LEU | A | 411 | −29.291 | 5.348 | 36.739 | 1.00 | 44.45 | C |
| ATOM | 389 | N | ALA | A | 412 | −30.680 | 5.890 | 31.376 | 1.00 | 45.18 | N |
| ATOM | 390 | CA | ALA | A | 412 | −30.467 | 5.420 | 30.012 | 1.00 | 45.24 | C |
| ATOM | 391 | C | ALA | A | 412 | −29.199 | 6.028 | 29.406 | 1.00 | 45.34 | C |
| ATOM | 392 | O | ALA | A | 412 | −28.593 | 5.430 | 28.520 | 1.00 | 45.28 | O |
| ATOM | 393 | CB | ALA | A | 412 | −31.677 | 5.726 | 29.148 | 1.00 | 45.00 | C |
| ATOM | 394 | N | GLU | A | 413 | −28.802 | 7.207 | 29.889 | 1.00 | 45.48 | N |
| ATOM | 395 | CA | GLU | A | 413 | −27.595 | 7.877 | 29.394 | 1.00 | 45.89 | C |
| ATOM | 396 | C | GLU | A | 413 | −26.543 | 8.153 | 30.481 | 1.00 | 45.94 | C |
| ATOM | 397 | O | GLU | A | 413 | −25.711 | 9.058 | 30.342 | 1.00 | 45.96 | O |
| ATOM | 398 | CB | GLU | A | 413 | −27.941 | 9.142 | 28.591 | 1.00 | 45.78 | C |
| ATOM | 399 | CG | GLU | A | 413 | −28.738 | 10.201 | 29.345 | 1.00 | 46.17 | C |
| ATOM | 400 | CD | GLU | A | 413 | −28.876 | 11.513 | 28.574 | 1.00 | 46.48 | C |
| ATOM | 401 | OE1 | GLU | A | 413 | −28.235 | 11.667 | 27.509 | 1.00 | 47.61 | O |
| ATOM | 402 | OE2 | GLU | A | 413 | −29.630 | 12.398 | 29.040 | 1.00 | 46.96 | O |
| ATOM | 403 | N | ASP | A | 414 | −26.586 | 7.359 | 31.552 | 1.00 | 46.08 | N |
| ATOM | 404 | CA | ASP | A | 414 | −25.537 | 7.329 | 32.574 | 1.00 | 46.10 | C |
| ATOM | 405 | C | ASP | A | 414 | −24.191 | 7.087 | 31.897 | 1.00 | 45.81 | C |
| ATOM | 406 | O | ASP | A | 414 | −24.110 | 6.300 | 30.960 | 1.00 | 45.74 | O |
| ATOM | 407 | CB | ASP | A | 414 | −25.829 | 6.209 | 33.581 | 1.00 | 46.31 | C |
| ATOM | 408 | CG | ASP | A | 414 | −24.917 | 6.247 | 34.807 | 1.00 | 47.44 | C |
| ATOM | 409 | OD1 | ASP | A | 414 | −25.427 | 5.999 | 35.924 | 1.00 | 48.43 | O |
| ATOM | 410 | OD2 | ASP | A | 414 | −23.699 | 6.516 | 34.668 | 1.00 | 48.58 | O |
| ATOM | 411 | N | ALA | A | 415 | −23.142 | 7.754 | 32.374 | 1.00 | 45.66 | N |
| ATOM | 412 | CA | ALA | A | 415 | −21.812 | 7.672 | 31.745 | 1.00 | 45.49 | C |
| ATOM | 413 | C | ALA | A | 415 | −21.140 | 6.300 | 31.877 | 1.00 | 45.25 | C |
| ATOM | 414 | O | ALA | A | 415 | −20.257 | 5.958 | 31.090 | 1.00 | 45.20 | O |
| ATOM | 415 | CB | ALA | A | 415 | −20.893 | 8.776 | 32.277 | 1.00 | 45.35 | C |
| ATOM | 416 | N | LYS | A | 416 | −21.565 | 5.525 | 32.870 | 1.00 | 45.10 | N |
| ATOM | 417 | CA | LYS | A | 416 | −21.023 | 4.188 | 33.113 | 1.00 | 44.91 | C |
| ATOM | 418 | C | LYS | A | 416 | −21.794 | 3.130 | 32.323 | 1.00 | 44.57 | C |
| ATOM | 419 | O | LYS | A | 416 | −22.901 | 2.748 | 32.696 | 1.00 | 44.41 | O |
| ATOM | 420 | CB | LYS | A | 416 | −21.031 | 3.885 | 34.618 | 1.00 | 45.04 | C |
| ATOM | 421 | CG | LYS | A | 416 | −19.722 | 4.217 | 35.361 | 1.00 | 45.39 | C |
| ATOM | 422 | CD | LYS | A | 416 | −19.155 | 5.614 | 35.047 | 1.00 | 45.58 | C |
| ATOM | 423 | CE | LYS | A | 416 | −17.959 | 5.970 | 35.938 | 1.00 | 45.30 | C |
| ATOM | 424 | NZ | LYS | A | 416 | −16.779 | 5.082 | 35.729 | 1.00 | 44.57 | N |
| ATOM | 425 | N | TRP | A | 417 | −21.193 | 2.658 | 31.233 | 1.00 | 44.46 | N |
| ATOM | 426 | CA | TRP | A | 417 | −21.891 | 1.821 | 30.242 | 1.00 | 44.33 | C |
| ATOM | 427 | C | TRP | A | 417 | −22.628 | 0.604 | 30.815 | 1.00 | 44.29 | C |
| ATOM | 428 | O | TRP | A | 417 | −23.670 | 0.210 | 30.294 | 1.00 | 44.23 | O |
| ATOM | 429 | CB | TRP | A | 417 | −20.961 | 1.431 | 29.072 | 1.00 | 44.21 | C |
| ATOM | 430 | CG | TRP | A | 417 | −19.988 | 0.323 | 29.356 | 1.00 | 43.94 | C |
| ATOM | 431 | CD1 | TRP | A | 417 | −18.677 | 0.457 | 29.717 | 1.00 | 43.54 | C |
| ATOM | 432 | CD2 | TRP | A | 417 | −20.246 | −1.088 | 29.295 | 1.00 | 44.06 | C |
| ATOM | 433 | NE1 | TRP | A | 417 | −18.105 | −0.781 | 29.892 | 1.00 | 43.19 | N |
| ATOM | 434 | CE2 | TRP | A | 417 | −19.045 | −1.747 | 29.643 | 1.00 | 43.56 | C |
| ATOM | 435 | CE3 | TRP | A | 417 | −21.379 | −1.860 | 28.984 | 1.00 | 44.40 | C |
| ATOM | 436 | CZ2 | TRP | A | 417 | −18.939 | −3.142 | 29.683 | 1.00 | 43.70 | C |
| ATOM | 437 | CZ3 | TRP | A | 417 | −21.273 | −3.252 | 29.030 | 1.00 | 43.90 | C |
| ATOM | 438 | CH2 | TRP | A | 417 | −20.059 | −3.875 | 29.375 | 1.00 | 43.93 | C |
| ATOM | 439 | N | ARG | A | 418 | −22.082 | 0.019 | 31.879 | 1.00 | 44.46 | N |
| ATOM | 440 | CA | ARG | A | 418 | −22.719 | −1.103 | 32.587 | 1.00 | 44.58 | C |
| ATOM | 441 | C | ARG | A | 418 | −24.104 | −0.736 | 33.121 | 1.00 | 44.35 | C |
| ATOM | 442 | O | ARG | A | 418 | −24.991 | −1.583 | 33.184 | 1.00 | 44.28 | O |
| ATOM | 443 | CB | ARG | A | 418 | −21.841 | −1.569 | 33.747 | 1.00 | 44.77 | C |
| ATOM | 444 | CG | ARG | A | 418 | −20.464 | −2.056 | 33.340 | 1.00 | 45.42 | C |
| ATOM | 445 | CD | ARG | A | 418 | −20.323 | −3.553 | 33.514 | 1.00 | 46.25 | C |
| ATOM | 446 | NE | ARG | A | 418 | −19.210 | −4.032 | 32.706 | 1.00 | 48.14 | N |
| ATOM | 447 | CZ | ARG | A | 418 | −18.723 | −5.267 | 32.730 | 1.00 | 48.91 | C |
| ATOM | 448 | NH1 | ARG | A | 418 | −19.240 | −6.181 | 33.549 | 1.00 | 48.49 | N |
| ATOM | 449 | NH2 | ARG | A | 418 | −17.708 | −5.581 | 31.927 | 1.00 | 48.96 | N |
| ATOM | 450 | N | VAL | A | 419 | −24.274 | 0.527 | 33.503 | 1.00 | 44.20 | N |
| ATOM | 451 | CA | VAL | A | 419 | −25.561 | 1.047 | 33.959 | 1.00 | 44.22 | C |
| ATOM | 452 | C | VAL | A | 419 | −26.531 | 1.219 | 32.785 | 1.00 | 44.19 | C |
| ATOM | 453 | O | VAL | A | 419 | −27.738 | 1.018 | 32.941 | 1.00 | 44.14 | O |
| ATOM | 454 | CB | VAL | A | 419 | −25.401 | 2.380 | 34.729 | 1.00 | 44.18 | C |
| ATOM | 455 | CG1 | VAL | A | 419 | −26.744 | 2.903 | 35.200 | 1.00 | 44.13 | C |
| ATOM | 456 | CG2 | VAL | A | 419 | −24.472 | 2.193 | 35.913 | 1.00 | 44.44 | C |
| ATOM | 457 | N | ARG | A | 420 | −26.008 | 1.583 | 31.615 | 1.00 | 44.16 | N |
| ATOM | 458 | CA | ARG | A | 420 | −26.835 | 1.629 | 30.409 | 1.00 | 44.23 | C |
| ATOM | 459 | C | ARG | A | 420 | −27.289 | 0.218 | 30.041 | 1.00 | 44.36 | C |
| ATOM | 460 | O | ARG | A | 420 | −28.450 | 0.014 | 29.689 | 1.00 | 44.45 | O |
| ATOM | 461 | CB | ARG | A | 420 | −26.114 | 2.308 | 29.239 | 1.00 | 44.13 | C |
| ATOM | 462 | CG | ARG | A | 420 | −25.758 | 3.767 | 29.501 | 1.00 | 44.31 | C |
| ATOM | 463 | CD | ARG | A | 420 | −25.428 | 4.546 | 28.224 | 1.00 | 44.11 | C |
| ATOM | 464 | NE | ARG | A | 420 | −24.192 | 4.123 | 27.566 | 1.00 | 44.23 | N |
| ATOM | 465 | CZ | ARG | A | 420 | −22.958 | 4.457 | 27.953 | 1.00 | 44.20 | C |
| ATOM | 466 | NH1 | ARG | A | 420 | −22.753 | 5.213 | 29.020 | 1.00 | 43.89 | N |
| ATOM | 467 | NH2 | ARG | A | 420 | −21.914 | 4.019 | 27.267 | 1.00 | 45.02 | N |
| ATOM | 468 | N | LEU | A | 421 | −26.381 | −0.751 | 30.160 | 1.00 | 44.59 | N |
| ATOM | 469 | CA | LEU | A | 421 | −26.717 | −2.164 | 29.962 | 1.00 | 44.74 | C |
| ATOM | 470 | C | LEU | A | 421 | −27.885 | −2.602 | 30.857 | 1.00 | 44.93 | C |
| ATOM | 471 | O | LEU | A | 421 | −28.833 | −3.238 | 30.383 | 1.00 | 45.01 | O |
| ATOM | 472 | CB | LEU | A | 421 | −25.492 | −3.068 | 30.195 | 1.00 | 44.78 | C |
| ATOM | 473 | CG | LEU | A | 421 | −25.637 | −4.562 | 29.842 | 1.00 | 44.85 | C |
| ATOM | 474 | CD1 | LEU | A | 421 | −25.767 | −4.756 | 28.329 | 1.00 | 44.86 | C |
| ATOM | 475 | CD2 | LEU | A | 421 | −24.487 | −5.411 | 30.385 | 1.00 | 44.44 | C |
| ATOM | 476 | N | ALA | A | 422 | −27.815 | −2.242 | 32.141 | 1.00 | 44.95 | N |
| ATOM | 477 | CA | ALA | A | 422 | −28.848 | −2.587 | 33.126 | 1.00 | 44.99 | C |
| ATOM | 478 | C | ALA | A | 422 | −30.229 | −2.026 | 32.777 | 1.00 | 45.07 | C |
| ATOM | 479 | O | ALA | A | 422 | −31.248 | −2.610 | 33.145 | 1.00 | 44.96 | O |
| ATOM | 480 | CB | ALA | A | 422 | −28.426 | −2.142 | 34.517 | 1.00 | 44.91 | C |
| ATOM | 481 | N | ILE | A | 423 | −30.257 | −0.896 | 32.074 | 1.00 | 45.24 | N |
| ATOM | 482 | CA | ILE | A | 423 | −31.515 | −0.330 | 31.590 | 1.00 | 45.42 | C |
| ATOM | 483 | C | ILE | A | 423 | −31.999 | −1.061 | 30.337 | 1.00 | 45.37 | C |
| ATOM | 484 | O | ILE | A | 423 | −33.160 | −1.463 | 30.261 | 1.00 | 45.52 | O |
| ATOM | 485 | CB | ILE | A | 423 | −31.409 | 1.191 | 31.312 | 1.00 | 45.54 | C |
| ATOM | 486 | CG1 | ILE | A | 423 | −30.830 | 1.929 | 32.527 | 1.00 | 45.75 | C |
| ATOM | 487 | CG2 | ILE | A | 423 | −32.775 | 1.769 | 30.896 | 1.00 | 45.24 | C |
| ATOM | 488 | CD1 | ILE | A | 423 | −31.650 | 1.793 | 33.815 | 1.00 | 46.98 | C |
| ATOM | 489 | N | ILE | A | 424 | −31.106 | −1.234 | 29.367 | 1.00 | 45.22 | N |
| ATOM | 490 | CA | ILE | A | 424 | −31.441 | −1.918 | 28.120 | 1.00 | 45.08 | C |
| ATOM | 491 | C | ILE | A | 424 | −32.095 | −3.278 | 28.382 | 1.00 | 45.21 | C |
| ATOM | 492 | O | ILE | A | 424 | −33.123 | −3.604 | 27.779 | 1.00 | 45.34 | O |
| ATOM | 493 | CB | ILE | A | 424 | −30.200 | −2.062 | 27.204 | 1.00 | 45.01 | C |
| ATOM | 494 | CG1 | ILE | A | 424 | −29.652 | −0.677 | 26.858 | 1.00 | 44.27 | C |
| ATOM | 495 | CG2 | ILE | A | 424 | −30.536 | −2.867 | 25.931 | 1.00 | 44.76 | C |
| ATOM | 496 | CD1 | ILE | A | 424 | −28.277 | −0.679 | 26.265 | 1.00 | 44.23 | C |
| ATOM | 497 | N | GLU | A | 425 | −31.510 | −4.050 | 29.298 | 1.00 | 45.16 | N |
| ATOM | 498 | CA | GLU | A | 425 | −32.001 | −5.393 | 29.624 | 1.00 | 45.35 | C |
| ATOM | 499 | C | GLU | A | 425 | −33.337 | −5.369 | 30.375 | 1.00 | 45.05 | C |
| ATOM | 500 | O | GLU | A | 425 | −34.098 | −6.338 | 30.341 | 1.00 | 44.92 | O |
| ATOM | 501 | CB | GLU | A | 425 | −30.944 | −6.186 | 30.406 | 1.00 | 45.32 | C |
| ATOM | 502 | CG | GLU | A | 425 | −29.700 | −6.541 | 29.575 | 1.00 | 45.96 | C |
| ATOM | 503 | CD | GLU | A | 425 | −28.594 | −7.207 | 30.381 | 1.00 | 46.08 | C |
| ATOM | 504 | OE1 | GLU | A | 425 | −28.651 | −7.169 | 31.630 | 1.00 | 47.24 | O |
| ATOM | 505 | OE2 | GLU | A | 425 | −27.663 | −7.767 | 29.757 | 1.00 | 46.78 | O |
| ATOM | 506 | N | TYR | A | 426 | −33.613 | −4.248 | 31.034 | 1.00 | 44.79 | N |
| ATOM | 507 | CA | TYR | A | 426 | −34.848 | −4.057 | 31.769 | 1.00 | 44.62 | C |
| ATOM | 508 | C | TYR | A | 426 | −35.966 | −3.538 | 30.861 | 1.00 | 44.57 | C |
| ATOM | 509 | O | TYR | A | 426 | −37.150 | −3.665 | 31.187 | 1.00 | 44.46 | O |
| ATOM | 510 | CB | TYR | A | 426 | −34.604 | −3.105 | 32.945 | 1.00 | 44.70 | C |
| ATOM | 511 | CG | TYR | A | 426 | −35.859 | −2.535 | 33.572 | 1.00 | 45.05 | C |
| ATOM | 512 | CD1 | TYR | A | 426 | −36.669 | −3.314 | 34.403 | 1.00 | 44.97 | C |
| ATOM | 513 | CD2 | TYR | A | 426 | −36.239 | −1.215 | 33.331 | 1.00 | 45.07 | C |
| ATOM | 514 | CE1 | TYR | A | 426 | −37.823 | −2.790 | 34.974 | 1.00 | 44.76 | C |
| ATOM | 515 | CE2 | TYR | A | 426 | −37.392 | −0.683 | 33.902 | 1.00 | 44.91 | C |
| ATOM | 516 | CZ | TYR | A | 426 | −38.176 | −1.475 | 34.718 | 1.00 | 44.68 | C |
| ATOM | 517 | OH | TYR | A | 426 | −39.317 | −0.947 | 35.275 | 1.00 | 45.12 | O |
| ATOM | 518 | N | MET | A | 427 | −35.589 | −2.972 | 29.717 | 1.00 | 44.55 | N |
| ATOM | 519 | CA | MET | A | 427 | −36.555 | −2.334 | 28.818 | 1.00 | 44.56 | C |
| ATOM | 520 | C | MET | A | 427 | −37.738 | −3.217 | 28.393 | 1.00 | 44.53 | C |
| ATOM | 521 | O | MET | A | 427 | −38.881 | −2.789 | 28.530 | 1.00 | 44.54 | O |
| ATOM | 522 | CB | MET | A | 427 | −35.872 | −1.726 | 27.584 | 1.00 | 44.62 | C |
| ATOM | 523 | CG | MET | A | 427 | −36.789 | −0.809 | 26.761 | 1.00 | 44.79 | C |
| ATOM | 524 | SD | MET | A | 427 | −36.614 | 0.978 | 27.054 | 1.00 | 45.86 | S |
| ATOM | 525 | CE | MET | A | 427 | −36.385 | 1.104 | 28.831 | 1.00 | 45.21 | C |
| ATOM | 526 | N | PRO | A | 428 | −37.476 | −4.440 | 27.874 | 1.00 | 44.54 | N |
| ATOM | 527 | CA | PRO | A | 428 | −38.610 | −5.231 | 27.380 | 1.00 | 44.54 | C |
| ATOM | 528 | C | PRO | A | 428 | −39.717 | −5.452 | 28.421 | 1.00 | 44.66 | C |
| ATOM | 529 | O | PRO | A | 428 | −40.899 | −5.327 | 28.086 | 1.00 | 44.75 | O |
| ATOM | 530 | CB | PRO | A | 428 | −37.969 | −6.558 | 26.946 | 1.00 | 44.53 | C |
| ATOM | 531 | CG | PRO | A | 428 | −36.606 | −6.563 | 27.529 | 1.00 | 44.53 | C |
| ATOM | 532 | CD | PRO | A | 428 | −36.194 | −5.147 | 27.689 | 1.00 | 44.44 | C |
| ATOM | 533 | N | LEU | A | 429 | −39.339 | −5.745 | 29.666 | 1.00 | 44.70 | N |
| ATOM | 534 | CA | LEU | A | 429 | −40.310 | −5.921 | 30.755 | 1.00 | 44.83 | C |
| ATOM | 535 | C | LEU | A | 429 | −41.058 | −4.620 | 31.053 | 1.00 | 44.83 | C |
| ATOM | 536 | O | LEU | A | 429 | −42.245 | −4.649 | 31.381 | 1.00 | 45.06 | O |
| ATOM | 537 | CB | LEU | A | 429 | −39.635 | −6.444 | 32.029 | 1.00 | 44.92 | C |
| ATOM | 538 | CG | LEU | A | 429 | −38.530 | −7.502 | 31.893 | 1.00 | 45.36 | C |
| ATOM | 539 | CD1 | LEU | A | 429 | −37.615 | −7.479 | 33.109 | 1.00 | 45.26 | C |
| ATOM | 540 | CD2 | LEU | A | 429 | −39.100 | −8.906 | 31.650 | 1.00 | 45.59 | C |
| ATOM | 541 | N | LEU | A | 430 | −40.361 | −3.489 | 30.932 | 1.00 | 44.79 | N |
| ATOM | 542 | CA | LEU | A | 430 | −40.973 | −2.167 | 31.103 | 1.00 | 44.77 | C |
| ATOM | 543 | C | LEU | A | 430 | −41.892 | −1.816 | 29.940 | 1.00 | 44.96 | C |
| ATOM | 544 | O | LEU | A | 430 | −43.055 | −1.463 | 30.154 | 1.00 | 45.13 | O |
| ATOM | 545 | CB | LEU | A | 430 | −39.912 | −1.070 | 31.277 | 1.00 | 44.76 | C |
| ATOM | 546 | CG | LEU | A | 430 | −40.363 | 0.396 | 31.160 | 1.00 | 44.55 | C |
| ATOM | 547 | CD1 | LEU | A | 430 | −41.373 | 0.783 | 32.243 | 1.00 | 44.84 | C |
| ATOM | 548 | CD2 | LEU | A | 430 | −39.173 | 1.330 | 31.193 | 1.00 | 44.47 | C |
| ATOM | 549 | N | ALA | A | 431 | −41.362 | −1.904 | 28.720 | 1.00 | 44.99 | N |
| ATOM | 550 | CA | ALA | A | 431 | −42.137 | −1.652 | 27.508 | 1.00 | 45.08 | C |
| ATOM | 551 | C | ALA | A | 431 | −43.393 | −2.524 | 27.481 | 1.00 | 45.23 | C |
| ATOM | 552 | O | ALA | A | 431 | −44.422 | −2.118 | 26.939 | 1.00 | 45.32 | O |
| ATOM | 553 | CB | ALA | A | 431 | −41.291 | −1.894 | 26.273 | 1.00 | 44.96 | C |
| ATOM | 554 | N | GLY | A | 432 | −43.303 | −3.715 | 28.077 | 1.00 | 45.19 | N |
| ATOM | 555 | CA | GLY | A | 432 | −44.458 | −4.584 | 28.255 | 1.00 | 45.06 | C |
| ATOM | 556 | C | GLY | A | 432 | −45.568 | −3.899 | 29.032 | 1.00 | 45.08 | C |
| ATOM | 557 | O | GLY | A | 432 | −46.673 | −3.726 | 28.520 | 1.00 | 45.03 | O |
| ATOM | 558 | N | GLN | A | 433 | −45.261 | −3.479 | 30.258 | 1.00 | 45.16 | N |
| ATOM | 559 | CA | GLN | A | 433 | −46.267 | −2.920 | 31.176 | 1.00 | 45.13 | C |
| ATOM | 560 | C | GLN | A | 433 | −46.806 | −1.549 | 30.784 | 1.00 | 44.87 | C |
| ATOM | 561 | O | GLN | A | 433 | −47.848 | −1.135 | 31.284 | 1.00 | 44.83 | O |
| ATOM | 562 | CB | GLN | A | 433 | −45.734 | −2.888 | 32.611 | 1.00 | 45.30 | C |
| ATOM | 563 | CG | GLN | A | 433 | −45.864 | −4.221 | 33.338 | 1.00 | 46.04 | C |
| ATOM | 564 | CD | GLN | A | 433 | −44.721 | −4.477 | 34.306 | 1.00 | 47.10 | C |
| ATOM | 565 | OE1 | GLN | A | 433 | −43.544 | −4.363 | 33.947 | 1.00 | 47.25 | O |
| ATOM | 566 | NE2 | GLN | A | 433 | −45.063 | −4.839 | 35.541 | 1.00 | 47.22 | N |
| ATOM | 567 | N | LEU | A | 434 | −46.099 | −0.842 | 29.906 | 1.00 | 44.68 | N |
| ATOM | 568 | CA | LEU | A | 434 | −46.623 | 0.404 | 29.350 | 1.00 | 44.53 | C |
| ATOM | 569 | C | LEU | A | 434 | −47.537 | 0.142 | 28.160 | 1.00 | 44.36 | C |
| ATOM | 570 | O | LEU | A | 434 | −48.573 | 0.794 | 28.016 | 1.00 | 44.28 | O |
| ATOM | 571 | CB | LEU | A | 434 | −45.500 | 1.363 | 28.945 | 1.00 | 44.53 | C |
| ATOM | 572 | CG | LEU | A | 434 | −44.808 | 2.229 | 30.004 | 1.00 | 44.81 | C |
| ATOM | 573 | CD1 | LEU | A | 434 | −44.179 | 3.425 | 29.314 | 1.00 | 45.19 | C |
| ATOM | 574 | CD2 | LEU | A | 434 | −45.745 | 2.705 | 31.118 | 1.00 | 44.95 | C |
| ATOM | 575 | N | GLY | A | 435 | −47.141 | −0.810 | 27.317 | 1.00 | 44.16 | N |
| ATOM | 576 | CA | GLY | A | 435 | −47.894 | −1.162 | 26.120 | 1.00 | 44.10 | C |
| ATOM | 577 | C | GLY | A | 435 | −47.424 | −0.427 | 24.881 | 1.00 | 44.09 | C |
| ATOM | 578 | O | GLY | A | 435 | −46.794 | 0.623 | 24.971 | 1.00 | 43.97 | O |
| ATOM | 579 | N | VAL | A | 436 | −47.754 | −0.991 | 23.722 | 1.00 | 44.37 | N |
| ATOM | 580 | CA | VAL | A | 436 | −47.385 | −0.451 | 22.407 | 1.00 | 44.65 | C |
| ATOM | 581 | C | VAL | A | 436 | −47.667 | 1.055 | 22.223 | 1.00 | 45.00 | C |
| ATOM | 582 | O | VAL | A | 436 | −46.823 | 1.786 | 21.699 | 1.00 | 45.11 | O |
| ATOM | 583 | CB | VAL | A | 436 | −48.035 | −1.298 | 21.252 | 1.00 | 44.62 | C |
| ATOM | 584 | CG1 | VAL | A | 436 | −49.552 | −1.438 | 21.437 | 1.00 | 44.34 | C |
| ATOM | 585 | CG2 | VAL | A | 436 | −47.695 | −0.740 | 19.874 | 1.00 | 44.44 | C |
| ATOM | 586 | N | GLU | A | 437 | −48.835 | 1.509 | 22.677 | 1.00 | 45.34 | N |
| ATOM | 587 | CA | GLU | A | 437 | −49.305 | 2.880 | 22.427 | 1.00 | 45.63 | C |
| ATOM | 588 | C | GLU | A | 437 | −48.532 | 3.975 | 23.176 | 1.00 | 45.91 | C |
| ATOM | 589 | O | GLU | A | 437 | −48.643 | 5.158 | 22.839 | 1.00 | 45.90 | O |
| ATOM | 590 | CB | GLU | A | 437 | −50.806 | 2.991 | 22.729 | 1.00 | 45.57 | C |
| ATOM | 591 | CG | GLU | A | 437 | −51.666 | 1.840 | 22.189 | 1.00 | 45.55 | C |
| ATOM | 592 | CD | GLU | A | 437 | −51.823 | 1.839 | 20.671 | 1.00 | 45.48 | C |
| ATOM | 593 | OE1 | GLU | A | 437 | −50.953 | 2.394 | 19.964 | 1.00 | 45.85 | O |
| ATOM | 594 | OE2 | GLU | A | 437 | −52.822 | 1.265 | 20.181 | 1.00 | 44.95 | O |
| ATOM | 595 | N | PHE | A | 438 | −47.758 | 3.574 | 24.184 | 1.00 | 46.25 | N |
| ATOM | 596 | CA | PHE | A | 438 | −46.957 | 4.496 | 24.990 | 1.00 | 46.61 | C |
| ATOM | 597 | C | PHE | A | 438 | −45.462 | 4.253 | 24.794 | 1.00 | 47.01 | C |
| ATOM | 598 | O | PHE | A | 438 | −44.626 | 4.780 | 25.537 | 1.00 | 46.88 | O |
| ATOM | 599 | CB | PHE | A | 438 | −47.338 | 4.374 | 26.470 | 1.00 | 46.50 | C |
| ATOM | 600 | CG | PHE | A | 438 | −48.672 | 4.971 | 26.797 | 1.00 | 46.59 | C |
| ATOM | 601 | CD1 | PHE | A | 438 | −49.845 | 4.240 | 26.613 | 1.00 | 46.78 | C |
| ATOM | 602 | CD2 | PHE | A | 438 | −48.762 | 6.272 | 27.278 | 1.00 | 46.59 | C |
| ATOM | 603 | CE1 | PHE | A | 438 | −51.087 | 4.798 | 26.906 | 1.00 | 46.58 | C |
| ATOM | 604 | CE2 | PHE | A | 438 | −49.999 | 6.837 | 27.574 | 1.00 | 46.73 | C |
| ATOM | 605 | CZ | PHE | A | 438 | −51.161 | 6.100 | 27.388 | 1.00 | 46.55 | C |
| ATOM | 606 | N | PHE | A | 439 | −45.135 | 3.455 | 23.782 | 1.00 | 47.60 | N |
| ATOM | 607 | CA | PHE | A | 439 | −43.748 | 3.130 | 23.480 | 1.00 | 48.21 | C |
| ATOM | 608 | C | PHE | A | 439 | −43.149 | 4.073 | 22.441 | 1.00 | 48.43 | C |
| ATOM | 609 | O | PHE | A | 439 | −42.033 | 4.567 | 22.617 | 1.00 | 48.60 | O |
| ATOM | 610 | CB | PHE | A | 439 | −43.597 | 1.673 | 23.031 | 1.00 | 48.30 | C |
| ATOM | 611 | CG | PHE | A | 439 | −42.184 | 1.302 | 22.690 | 1.00 | 48.69 | C |
| ATOM | 612 | CD1 | PHE | A | 439 | −41.300 | 0.899 | 23.686 | 1.00 | 48.97 | C |
| ATOM | 613 | CD2 | PHE | A | 439 | −41.724 | 1.389 | 21.376 | 1.00 | 49.30 | C |
| ATOM | 614 | CE1 | PHE | A | 439 | −39.981 | 0.570 | 23.376 | 1.00 | 49.30 | C |
| ATOM | 615 | CE2 | PHE | A | 439 | −40.408 | 1.064 | 21.055 | 1.00 | 49.28 | C |
| ATOM | 616 | CZ | PHE | A | 439 | −39.536 | 0.654 | 22.057 | 1.00 | 49.09 | C |
| ATOM | 617 | N | ASP | A | 440 | −43.886 | 4.309 | 21.359 | 1.00 | 48.78 | N |
| ATOM | 618 | CA | ASP | A | 440 | −43.456 | 5.232 | 20.306 | 1.00 | 49.20 | C |
| ATOM | 619 | C | ASP | A | 440 | −43.287 | 6.645 | 20.876 | 1.00 | 49.41 | C |
| ATOM | 620 | O | ASP | A | 440 | −42.600 | 7.490 | 20.297 | 1.00 | 49.35 | O |
| ATOM | 621 | CB | ASP | A | 440 | −44.459 | 5.221 | 19.148 | 1.00 | 49.22 | C |
| ATOM | 622 | CG | ASP | A | 440 | −44.906 | 3.809 | 18.773 | 1.00 | 49.33 | C |
| ATOM | 623 | OD1 | ASP | A | 440 | −44.042 | 2.984 | 18.395 | 1.00 | 49.43 | O |
| ATOM | 624 | OD2 | ASP | A | 440 | −46.123 | 3.527 | 18.859 | 1.00 | 48.71 | O |
| ATOM | 625 | N | GLU | A | 441 | −43.919 | 6.864 | 22.028 | 1.00 | 49.70 | N |
| ATOM | 626 | CA | GLU | A | 441 | −43.823 | 8.087 | 22.811 | 1.00 | 49.97 | C |
| ATOM | 627 | C | GLU | A | 441 | −42.408 | 8.294 | 23.368 | 1.00 | 50.20 | C |
| ATOM | 628 | O | GLU | A | 441 | −41.512 | 8.758 | 22.659 | 1.00 | 50.38 | O |
| ATOM | 629 | CB | GLU | A | 441 | −44.842 | 8.016 | 23.955 | 1.00 | 49.95 | C |
| ATOM | 630 | CG | GLU | A | 441 | −45.276 | 9.351 | 24.533 | 1.00 | 50.14 | C |
| ATOM | 631 | CD | GLU | A | 441 | −46.460 | 9.212 | 25.478 | 1.00 | 49.98 | C |
| ATOM | 632 | OE1 | GLU | A | 441 | −47.520 | 9.814 | 25.196 | 1.00 | 49.45 | O |
| ATOM | 633 | OE2 | GLU | A | 441 | −46.332 | 8.492 | 26.493 | 1.00 | 49.79 | O |
| ATOM | 634 | N | LYS | A | 442 | −42.211 | 7.922 | 24.630 | 1.00 | 50.31 | N |
| ATOM | 635 | CA | LYS | A | 442 | −40.992 | 8.253 | 25.365 | 1.00 | 50.41 | C |
| ATOM | 636 | C | LYS | A | 442 | −39.794 | 7.362 | 25.010 | 1.00 | 50.41 | C |
| ATOM | 637 | O | LYS | A | 442 | −38.656 | 7.837 | 24.918 | 1.00 | 50.47 | O |
| ATOM | 638 | CB | LYS | A | 442 | −41.256 | 8.180 | 26.878 | 1.00 | 50.48 | C |
| ATOM | 639 | CG | LYS | A | 442 | −42.660 | 8.602 | 27.331 | 1.00 | 50.55 | C |
| ATOM | 640 | CD | LYS | A | 442 | −42.853 | 10.116 | 27.297 | 1.00 | 50.83 | C |
| ATOM | 641 | CE | LYS | A | 442 | −44.165 | 10.521 | 27.959 | 1.00 | 50.77 | C |
| ATOM | 642 | NZ | LYS | A | 442 | −44.473 | 11.967 | 27.779 | 1.00 | 50.59 | N |
| ATOM | 643 | N | LEU | A | 443 | −40.066 | 6.078 | 24.792 | 1.00 | 50.19 | N |
| ATOM | 644 | CA | LEU | A | 443 | −39.029 | 5.046 | 24.814 | 1.00 | 50.02 | C |
| ATOM | 645 | C | LEU | A | 443 | −38.424 | 4.672 | 23.459 | 1.00 | 49.67 | C |
| ATOM | 646 | O | LEU | A | 443 | −37.324 | 4.116 | 23.408 | 1.00 | 49.49 | O |
| ATOM | 647 | CB | LEU | A | 443 | −39.556 | 3.789 | 25.522 | 1.00 | 50.11 | C |
| ATOM | 648 | CG | LEU | A | 443 | −39.948 | 3.890 | 27.003 | 1.00 | 50.24 | C |
| ATOM | 649 | CD1 | LEU | A | 443 | −41.323 | 4.520 | 27.186 | 1.00 | 50.56 | C |
| ATOM | 650 | CD2 | LEU | A | 443 | −39.929 | 2.514 | 27.643 | 1.00 | 50.40 | C |
| ATOM | 651 | N | ASN | A | 444 | −39.139 | 4.961 | 22.373 | 1.00 | 49.47 | N |
| ATOM | 652 | CA | ASN | A | 444 | −38.640 | 4.688 | 21.020 | 1.00 | 49.37 | C |
| ATOM | 653 | C | ASN | A | 444 | −37.292 | 5.358 | 20.739 | 1.00 | 49.14 | C |
| ATOM | 654 | O | ASN | A | 444 | −36.381 | 4.728 | 20.193 | 1.00 | 49.15 | O |
| ATOM | 655 | CB | ASN | A | 444 | −39.667 | 5.102 | 19.954 | 1.00 | 49.46 | C |
| ATOM | 656 | CG | ASN | A | 444 | −39.142 | 4.928 | 18.527 | 1.00 | 49.75 | C |
| ATOM | 657 | OD1 | ASN | A | 444 | −38.897 | 3.810 | 18.073 | 1.00 | 50.65 | O |
| ATOM | 658 | ND2 | ASN | A | 444 | −38.973 | 6.038 | 17.818 | 1.00 | 49.81 | N |
| ATOM | 659 | N | SER | A | 445 | −37.174 | 6.630 | 21.115 | 1.00 | 48.80 | N |
| ATOM | 660 | CA | SER | A | 445 | −35.934 | 7.376 | 20.919 | 1.00 | 48.57 | C |
| ATOM | 661 | C | SER | A | 445 | −34.797 | 6.796 | 21.765 | 1.00 | 48.07 | C |
| ATOM | 662 | O | SER | A | 445 | −33.679 | 6.652 | 21.281 | 1.00 | 48.00 | O |
| ATOM | 663 | CB | SER | A | 445 | −36.131 | 8.883 | 21.169 | 1.00 | 48.72 | C |
| ATOM | 664 | OG | SER | A | 445 | −36.668 | 9.147 | 22.456 | 1.00 | 49.32 | O |
| ATOM | 665 | N | LEU | A | 446 | −35.098 | 6.446 | 23.013 | 1.00 | 47.56 | N |
| ATOM | 666 | CA | LEU | A | 446 | −34.141 | 5.770 | 23.883 | 1.00 | 47.21 | C |
| ATOM | 667 | C | LEU | A | 446 | −33.624 | 4.480 | 23.258 | 1.00 | 47.02 | C |
| ATOM | 668 | O | LEU | A | 446 | −32.420 | 4.240 | 23.228 | 1.00 | 47.14 | O |
| ATOM | 669 | CB | LEU | A | 446 | −34.768 | 5.468 | 25.245 | 1.00 | 47.20 | C |
| ATOM | 670 | CG | LEU | A | 446 | −34.603 | 6.468 | 26.394 | 1.00 | 47.24 | C |
| ATOM | 671 | CD1 | LEU | A | 446 | −35.152 | 7.857 | 26.069 | 1.00 | 47.10 | C |
| ATOM | 672 | CD2 | LEU | A | 446 | −35.267 | 5.913 | 27.649 | 1.00 | 47.27 | C |
| ATOM | 673 | N | CYS | A | 447 | −34.537 | 3.658 | 22.754 | 1.00 | 46.73 | N |
| ATOM | 674 | CA | CYS | A | 447 | −34.159 | 2.403 | 22.126 | 1.00 | 46.57 | C |
| ATOM | 675 | C | CYS | A | 447 | −33.295 | 2.606 | 20.876 | 1.00 | 46.39 | C |
| ATOM | 676 | O | CYS | A | 447 | −32.304 | 1.900 | 20.689 | 1.00 | 46.51 | O |
| ATOM | 677 | CB | CYS | A | 447 | −35.396 | 1.552 | 21.817 | 1.00 | 46.74 | C |
| ATOM | 678 | SG | CYS | A | 447 | −36.174 | 0.784 | 23.286 | 1.00 | 46.68 | S |
| ATOM | 679 | N | MET | A | 448 | −33.655 | 3.577 | 20.039 | 1.00 | 46.06 | N |
| ATOM | 680 | CA | MET | A | 448 | −32.900 | 3.844 | 18.810 | 1.00 | 45.82 | C |
| ATOM | 681 | C | MET | A | 448 | −31.517 | 4.435 | 19.072 | 1.00 | 45.42 | C |
| ATOM | 682 | O | MET | A | 448 | −30.555 | 4.105 | 18.373 | 1.00 | 45.47 | O |
| ATOM | 683 | CB | MET | A | 448 | −33.671 | 4.775 | 17.867 | 1.00 | 46.02 | C |
| ATOM | 684 | CG | MET | A | 448 | −34.859 | 4.161 | 17.116 | 1.00 | 47.07 | C |
| ATOM | 685 | SD | MET | A | 448 | −34.716 | 2.426 | 16.618 | 1.00 | 48.70 | S |
| ATOM | 686 | CE | MET | A | 448 | −35.832 | 1.681 | 17.805 | 1.00 | 48.54 | C |
| ATOM | 687 | N | ALA | A | 449 | −31.426 | 5.313 | 20.070 | 1.00 | 44.90 | N |
| ATOM | 688 | CA | ALA | A | 449 | −30.188 | 6.047 | 20.365 | 1.00 | 44.23 | C |
| ATOM | 689 | C | ALA | A | 449 | −29.088 | 5.144 | 20.903 | 1.00 | 43.84 | C |
| ATOM | 690 | O | ALA | A | 449 | −27.903 | 5.437 | 20.716 | 1.00 | 43.73 | O |
| ATOM | 691 | CB | ALA | A | 449 | −30.452 | 7.196 | 21.333 | 1.00 | 44.18 | C |
| ATOM | 692 | N | TRP | A | 450 | −29.486 | 4.058 | 21.571 | 1.00 | 43.19 | N |
| ATOM | 693 | CA | TRP | A | 450 | −28.545 | 3.061 | 22.066 | 1.00 | 42.67 | C |
| ATOM | 694 | C | TRP | A | 450 | −27.835 | 2.332 | 20.935 | 1.00 | 42.51 | C |
| ATOM | 695 | O | TRP | A | 450 | −26.696 | 1.905 | 21.086 | 1.00 | 42.61 | O |
| ATOM | 696 | CB | TRP | A | 450 | −29.247 | 2.036 | 22.952 | 1.00 | 42.51 | C |
| ATOM | 697 | CG | TRP | A | 450 | −29.616 | 2.524 | 24.319 | 1.00 | 42.57 | C |
| ATOM | 698 | CD1 | TRP | A | 450 | −28.946 | 3.441 | 25.076 | 1.00 | 42.29 | C |
| ATOM | 699 | CD2 | TRP | A | 450 | −30.734 | 2.092 | 25.108 | 1.00 | 42.39 | C |
| ATOM | 700 | NE1 | TRP | A | 450 | −29.582 | 3.618 | 26.280 | 1.00 | 42.32 | N |
| ATOM | 701 | CE2 | TRP | A | 450 | −30.683 | 2.803 | 26.328 | 1.00 | 42.24 | C |
| ATOM | 702 | CE3 | TRP | A | 450 | −31.777 | 1.181 | 24.899 | 1.00 | 41.84 | C |
| ATOM | 703 | CZ2 | TRP | A | 450 | −31.630 | 2.629 | 27.337 | 1.00 | 41.94 | C |
| ATOM | 704 | CZ3 | TRP | A | 450 | −32.717 | 1.009 | 25.901 | 1.00 | 42.41 | C |
| ATOM | 705 | CH2 | TRP | A | 450 | −32.637 | 1.733 | 27.107 | 1.00 | 42.38 | C |
| ATOM | 706 | N | LEU | A | 451 | −28.515 | 2.185 | 19.804 | 1.00 | 42.46 | N |
| ATOM | 707 | CA | LEU | A | 451 | −27.953 | 1.497 | 18.645 | 1.00 | 42.32 | C |
| ATOM | 708 | C | LEU | A | 451 | −26.704 | 2.204 | 18.148 | 1.00 | 42.32 | C |
| ATOM | 709 | O | LEU | A | 451 | −25.875 | 1.605 | 17.466 | 1.00 | 42.53 | O |
| ATOM | 710 | CB | LEU | A | 451 | −28.978 | 1.439 | 17.511 | 1.00 | 42.12 | C |
| ATOM | 711 | CG | LEU | A | 451 | −30.201 | 0.538 | 17.656 | 1.00 | 41.82 | C |
| ATOM | 712 | CD1 | LEU | A | 451 | −31.243 | 0.959 | 16.643 | 1.00 | 42.35 | C |
| ATOM | 713 | CD2 | LEU | A | 451 | −29.844 | −0.931 | 17.474 | 1.00 | 41.32 | C |
| ATOM | 714 | N | VAL | A | 452 | −26.582 | 3.477 | 18.508 | 1.00 | 42.11 | N |
| ATOM | 715 | CA | VAL | A | 452 | −25.570 | 4.360 | 17.956 | 1.00 | 41.95 | C |
| ATOM | 716 | C | VAL | A | 452 | −24.629 | 4.881 | 19.066 | 1.00 | 42.16 | C |
| ATOM | 717 | O | VAL | A | 452 | −23.781 | 5.751 | 18.830 | 1.00 | 42.12 | O |
| ATOM | 718 | CB | VAL | A | 452 | −26.257 | 5.477 | 17.115 | 1.00 | 41.75 | C |
| ATOM | 719 | CG1 | VAL | A | 452 | −26.517 | 6.735 | 17.929 | 1.00 | 41.29 | C |
| ATOM | 720 | CG2 | VAL | A | 452 | −25.457 | 5.776 | 15.890 | 1.00 | 41.94 | C |
| ATOM | 721 | N | ASP | A | 453 | −24.793 | 4.307 | 20.265 | 1.00 | 42.36 | N |
| ATOM | 722 | CA | ASP | A | 453 | −23.925 | 4.502 | 21.441 | 1.00 | 42.40 | C |
| ATOM | 723 | C | ASP | A | 453 | −22.458 | 4.282 | 21.095 | 1.00 | 42.60 | C |
| ATOM | 724 | O | ASP | A | 453 | −22.131 | 3.506 | 20.202 | 1.00 | 42.66 | O |
| ATOM | 725 | CB | ASP | A | 453 | −24.336 | 3.505 | 22.537 | 1.00 | 42.23 | C |
| ATOM | 726 | CG | ASP | A | 453 | −23.790 | 3.861 | 23.928 | 1.00 | 42.34 | C |
| ATOM | 727 | OD1 | ASP | A | 453 | −22.556 | 3.812 | 24.152 | 1.00 | 41.61 | O |
| ATOM | 728 | OD2 | ASP | A | 453 | −24.620 | 4.142 | 24.823 | 1.00 | 41.93 | O |
| ATOM | 729 | N | HIS | A | 454 | −21.572 | 4.953 | 21.821 | 1.00 | 42.92 | N |
| ATOM | 730 | CA | HIS | A | 454 | −20.141 | 4.826 | 21.583 | 1.00 | 43.13 | C |
| ATOM | 731 | C | HIS | A | 454 | −19.576 | 3.447 | 21.944 | 1.00 | 43.26 | C |
| ATOM | 732 | O | HIS | A | 454 | −18.614 | 2.998 | 21.325 | 1.00 | 43.42 | O |
| ATOM | 733 | CB | HIS | A | 454 | −19.379 | 5.928 | 22.319 | 1.00 | 43.20 | C |
| ATOM | 734 | CG | HIS | A | 454 | −19.429 | 7.261 | 21.636 | 1.00 | 43.51 | C |
| ATOM | 735 | ND1 | HIS | A | 454 | −19.858 | 8.406 | 22.271 | 1.00 | 44.03 | N |
| ATOM | 736 | CD2 | HIS | A | 454 | −19.091 | 7.634 | 20.378 | 1.00 | 43.71 | C |
| ATOM | 737 | CE1 | HIS | A | 454 | −19.782 | 9.428 | 21.435 | 1.00 | 43.82 | C |
| ATOM | 738 | NE2 | HIS | A | 454 | −19.321 | 8.985 | 20.279 | 1.00 | 43.75 | N |
| ATOM | 739 | N | VAL | A | 455 | −20.173 | 2.788 | 22.937 | 1.00 | 43.46 | N |
| ATOM | 740 | CA | VAL | A | 455 | −19.714 | 1.478 | 23.414 | 1.00 | 43.70 | C |
| ATOM | 741 | C | VAL | A | 455 | −20.409 | 0.327 | 22.675 | 1.00 | 43.95 | C |
| ATOM | 742 | O | VAL | A | 455 | −21.634 | 0.188 | 22.756 | 1.00 | 44.21 | O |
| ATOM | 743 | CB | VAL | A | 455 | −19.963 | 1.314 | 24.935 | 1.00 | 43.77 | C |
| ATOM | 744 | CG1 | VAL | A | 455 | −19.610 | −0.108 | 25.404 | 1.00 | 43.71 | C |
| ATOM | 745 | CG2 | VAL | A | 455 | −19.187 | 2.359 | 25.729 | 1.00 | 43.56 | C |
| ATOM | 746 | N | TYR | A | 456 | −19.624 | −0.505 | 21.986 | 1.00 | 43.89 | N |
| ATOM | 747 | CA | TYR | A | 456 | −20.156 | −1.643 | 21.229 | 1.00 | 43.87 | C |
| ATOM | 748 | C | TYR | A | 456 | −21.123 | −2.533 | 22.011 | 1.00 | 43.69 | C |
| ATOM | 749 | O | TYR | A | 456 | −22.149 | −2.947 | 21.478 | 1.00 | 43.53 | O |
| ATOM | 750 | CB | TYR | A | 456 | −19.037 | −2.528 | 20.680 | 1.00 | 44.24 | C |
| ATOM | 751 | CG | TYR | A | 456 | −19.567 | −3.861 | 20.172 | 1.00 | 45.11 | C |
| ATOM | 752 | CD1 | TYR | A | 456 | −19.564 | −4.997 | 20.989 | 1.00 | 44.18 | C |
| ATOM | 753 | CD2 | TYR | A | 456 | −20.117 | −3.973 | 18.884 | 1.00 | 45.19 | C |
| ATOM | 754 | CE1 | TYR | A | 456 | −20.071 | −6.213 | 20.532 | 1.00 | 44.52 | C |
| ATOM | 755 | CE2 | TYR | A | 456 | −20.625 | −5.189 | 18.417 | 1.00 | 44.86 | C |
| ATOM | 756 | CZ | TYR | A | 456 | −20.597 | −6.302 | 19.244 | 1.00 | 45.14 | C |
| ATOM | 757 | OH | TYR | A | 456 | −21.099 | −7.503 | 18.787 | 1.00 | 45.70 | O |
| ATOM | 758 | N | ALA | A | 457 | −20.774 | −2.852 | 23.255 | 1.00 | 43.42 | N |
| ATOM | 759 | CA | ALA | A | 457 | −21.601 | −3.721 | 24.081 | 1.00 | 43.35 | C |
| ATOM | 760 | C | ALA | A | 457 | −23.001 | −3.147 | 24.313 | 1.00 | 43.43 | C |
| ATOM | 761 | O | ALA | A | 457 | −23.942 | −3.896 | 24.571 | 1.00 | 43.35 | O |
| ATOM | 762 | CB | ALA | A | 457 | −20.913 | −4.028 | 25.400 | 1.00 | 43.21 | C |
| ATOM | 763 | N | ILE | A | 458 | −23.128 | −1.825 | 24.215 | 1.00 | 43.57 | N |
| ATOM | 764 | CA | ILE | A | 458 | −24.429 | −1.168 | 24.285 | 1.00 | 43.83 | C |
| ATOM | 765 | C | ILE | A | 458 | −25.190 | −1.346 | 22.967 | 1.00 | 44.23 | C |
| ATOM | 766 | O | ILE | A | 458 | −26.348 | −1.781 | 22.965 | 1.00 | 44.51 | O |
| ATOM | 767 | CB | ILE | A | 458 | −24.298 | 0.332 | 24.635 | 1.00 | 43.77 | C |
| ATOM | 768 | CG1 | ILE | A | 458 | −23.752 | 0.512 | 26.060 | 1.00 | 43.84 | C |
| ATOM | 769 | CG2 | ILE | A | 458 | −25.633 | 1.049 | 24.449 | 1.00 | 43.48 | C |
| ATOM | 770 | CD1 | ILE | A | 458 | −24.570 | −0.173 | 27.165 | 1.00 | 43.67 | C |
| ATOM | 771 | N | ARG | A | 459 | −24.527 | −1.024 | 21.854 | 1.00 | 44.35 | N |
| ATOM | 772 | CA | ARG | A | 459 | −25.081 | −1.223 | 20.519 | 1.00 | 44.45 | C |
| ATOM | 773 | C | ARG | A | 459 | −25.525 | −2.660 | 20.315 | 1.00 | 44.74 | C |
| ATOM | 774 | O | ARG | A | 459 | −26.556 | −2.914 | 19.706 | 1.00 | 45.10 | O |
| ATOM | 775 | CB | ARG | A | 459 | −24.054 | −0.854 | 19.454 | 1.00 | 44.51 | C |
| ATOM | 776 | CG | ARG | A | 459 | −23.528 | 0.567 | 19.568 | 1.00 | 44.00 | C |
| ATOM | 777 | CD | ARG | A | 459 | −22.944 | 1.052 | 18.259 | 1.00 | 43.82 | C |
| ATOM | 778 | NE | ARG | A | 459 | −21.727 | 0.344 | 17.856 | 1.00 | 44.55 | N |
| ATOM | 779 | CZ | ARG | A | 459 | −20.520 | 0.538 | 18.387 | 1.00 | 44.57 | C |
| ATOM | 780 | NH1 | ARG | A | 459 | −20.345 | 1.399 | 19.382 | 1.00 | 45.33 | N |
| ATOM | 781 | NH2 | ARG | A | 459 | −19.485 | −0.149 | 17.935 | 1.00 | 44.53 | N |
| ATOM | 782 | N | GLU | A | 460 | −24.745 | −3.591 | 20.850 | 1.00 | 45.13 | N |
| ATOM | 783 | CA | GLU | A | 460 | −25.023 | −5.018 | 20.740 | 1.00 | 45.24 | C |
| ATOM | 784 | C | GLU | A | 460 | −26.205 | −5.448 | 21.596 | 1.00 | 45.03 | C |
| ATOM | 785 | O | GLU | A | 460 | −27.021 | −6.259 | 21.159 | 1.00 | 45.26 | O |
| ATOM | 786 | CB | GLU | A | 460 | −23.794 | −5.823 | 21.133 | 1.00 | 45.32 | C |
| ATOM | 787 | CG | GLU | A | 460 | −23.808 | −7.233 | 20.615 | 1.00 | 47.02 | C |
| ATOM | 788 | CD | GLU | A | 460 | −23.423 | −8.228 | 21.679 | 1.00 | 49.77 | C |
| ATOM | 789 | OE1 | GLU | A | 460 | −24.116 | −9.268 | 21.772 | 1.00 | 52.13 | O |
| ATOM | 790 | OE2 | GLU | A | 460 | −22.453 | −7.966 | 22.434 | 1.00 | 49.37 | O |
| ATOM | 791 | N | ALA | A | 461 | −26.290 | −4.924 | 22.816 | 1.00 | 44.71 | N |
| ATOM | 792 | CA | ALA | A | 461 | −27.412 | −5.237 | 23.695 | 1.00 | 44.49 | C |
| ATOM | 793 | C | ALA | A | 461 | −28.701 | −4.623 | 23.154 | 1.00 | 44.63 | C |
| ATOM | 794 | O | ALA | A | 461 | −29.780 | −5.230 | 23.251 | 1.00 | 44.35 | O |
| ATOM | 795 | CB | ALA | A | 461 | −27.144 | −4.740 | 25.084 | 1.00 | 44.56 | C |
| ATOM | 796 | N | ALA | A | 462 | −28.567 | −3.419 | 22.582 | 1.00 | 44.45 | N |
| ATOM | 797 | CA | ALA | A | 462 | −29.681 | −2.679 | 21.992 | 1.00 | 44.19 | C |
| ATOM | 798 | C | ALA | A | 462 | −30.226 | −3.409 | 20.783 | 1.00 | 44.16 | C |
| ATOM | 799 | O | ALA | A | 462 | −31.412 | −3.329 | 20.472 | 1.00 | 44.06 | O |
| ATOM | 800 | CB | ALA | A | 462 | −29.240 | −1.297 | 21.602 | 1.00 | 44.14 | C |
| ATOM | 801 | N | THR | A | 463 | −29.339 | −4.115 | 20.099 | 1.00 | 44.38 | N |
| ATOM | 802 | CA | THR | A | 463 | −29.709 | −4.921 | 18.957 | 1.00 | 44.66 | C |
| ATOM | 803 | C | THR | A | 463 | −30.624 | −6.048 | 19.403 | 1.00 | 44.77 | C |
| ATOM | 804 | O | THR | A | 463 | −31.753 | −6.153 | 18.927 | 1.00 | 45.10 | O |
| ATOM | 805 | CB | THR | A | 463 | −28.459 | −5.447 | 18.253 | 1.00 | 44.64 | C |
| ATOM | 806 | OG1 | THR | A | 463 | −27.791 | −4.339 | 17.640 | 1.00 | 45.06 | O |
| ATOM | 807 | CG2 | THR | A | 463 | −28.814 | −6.480 | 17.190 | 1.00 | 44.75 | C |
| ATOM | 808 | N | SER | A | 464 | −30.154 | −6.860 | 20.347 | 1.00 | 44.79 | N |
| ATOM | 809 | CA | SER | A | 464 | −30.941 | −7.974 | 20.856 | 1.00 | 44.73 | C |
| ATOM | 810 | C | SER | A | 464 | −32.171 | −7.493 | 21.652 | 1.00 | 44.67 | C |
| ATOM | 811 | O | SER | A | 464 | −33.092 | −8.273 | 21.927 | 1.00 | 44.88 | O |
| ATOM | 812 | CB | SER | A | 464 | −30.059 | −8.918 | 21.686 | 1.00 | 44.66 | C |
| ATOM | 813 | OG | SER | A | 464 | −30.256 | −8.730 | 23.080 | 1.00 | 45.00 | O |
| ATOM | 814 | N | ASN | A | 465 | −32.178 | −6.211 | 22.011 | 1.00 | 44.41 | N |
| ATOM | 815 | CA | ASN | A | 465 | −33.318 | −5.606 | 22.694 | 1.00 | 44.42 | C |
| ATOM | 816 | C | ASN | A | 465 | −34.535 | −5.428 | 21.776 | 1.00 | 44.17 | C |
| ATOM | 817 | O | ASN | A | 465 | −35.684 | −5.638 | 22.196 | 1.00 | 43.98 | O |
| ATOM | 818 | CB | ASN | A | 465 | −32.919 | −4.264 | 23.313 | 1.00 | 44.65 | C |
| ATOM | 819 | CG | ASN | A | 465 | −34.095 | −3.542 | 23.954 | 1.00 | 45.33 | C |
| ATOM | 820 | OD1 | ASN | A | 465 | −34.414 | −2.412 | 23.576 | 1.00 | 46.04 | O |
| ATOM | 821 | ND2 | ASN | A | 465 | −34.752 | −4.197 | 24.921 | 1.00 | 44.64 | N |
| ATOM | 822 | N | LEU | A | 466 | −34.272 | −5.043 | 20.529 | 1.00 | 43.79 | N |
| ATOM | 823 | CA | LEU | A | 466 | −35.319 | −4.893 | 19.525 | 1.00 | 43.60 | C |
| ATOM | 824 | C | LEU | A | 466 | −36.066 | −6.207 | 19.308 | 1.00 | 43.61 | C |
| ATOM | 825 | O | LEU | A | 466 | −37.285 | −6.211 | 19.141 | 1.00 | 43.60 | O |
| ATOM | 826 | CB | LEU | A | 466 | −34.734 | −4.387 | 18.201 | 1.00 | 43.56 | C |
| ATOM | 827 | CG | LEU | A | 466 | −34.040 | −3.020 | 18.218 | 1.00 | 43.36 | C |
| ATOM | 828 | CD1 | LEU | A | 466 | −33.276 | −2.768 | 16.927 | 1.00 | 42.93 | C |
| ATOM | 829 | CD2 | LEU | A | 466 | −35.039 | −1.909 | 18.488 | 1.00 | 42.93 | C |
| ATOM | 830 | N | LYS | A | 467 | −35.323 | −7.315 | 19.320 | 1.00 | 43.51 | N |
| ATOM | 831 | CA | LYS | A | 467 | −35.893 | −8.647 | 19.160 | 1.00 | 43.24 | C |
| ATOM | 832 | C | LYS | A | 467 | −36.915 | −8.914 | 20.247 | 1.00 | 43.12 | C |
| ATOM | 833 | O | LYS | A | 467 | −38.031 | −9.354 | 19.968 | 1.00 | 43.30 | O |
| ATOM | 834 | CB | LYS | A | 467 | −34.798 | −9.712 | 19.217 | 1.00 | 43.27 | C |
| ATOM | 835 | CG | LYS | A | 467 | −35.267 | −11.095 | 18.783 | 1.00 | 43.73 | C |
| ATOM | 836 | CD | LYS | A | 467 | −34.254 | −12.180 | 19.113 | 1.00 | 44.14 | C |
| ATOM | 837 | CE | LYS | A | 467 | −34.863 | −13.552 | 18.887 | 1.00 | 44.95 | C |
| ATOM | 838 | NZ | LYS | A | 467 | −33.964 | −14.652 | 19.315 | 1.00 | 45.73 | N |
| ATOM | 839 | N | LYS | A | 468 | −36.531 | −8.637 | 21.487 | 1.00 | 42.92 | N |
| ATOM | 840 | CA | LYS | A | 468 | −37.412 | −8.856 | 22.621 | 1.00 | 42.76 | C |
| ATOM | 841 | C | LYS | A | 468 | −38.629 | −7.954 | 22.515 | 1.00 | 42.48 | C |
| ATOM | 842 | O | LYS | A | 468 | −39.725 | −8.326 | 22.926 | 1.00 | 42.46 | O |
| ATOM | 843 | CB | LYS | A | 468 | −36.657 | −8.635 | 23.932 | 1.00 | 42.94 | C |
| ATOM | 844 | CG | LYS | A | 468 | −35.744 | −9.798 | 24.300 | 1.00 | 43.58 | C |
| ATOM | 845 | CD | LYS | A | 468 | −34.694 | −9.394 | 25.309 | 1.00 | 45.20 | C |
| ATOM | 846 | CE | LYS | A | 468 | −33.671 | −10.505 | 25.512 | 1.00 | 46.32 | C |
| ATOM | 847 | NZ | LYS | A | 468 | −32.295 | −9.948 | 25.740 | 1.00 | 47.01 | N |
| ATOM | 848 | N | LEU | A | 469 | −38.434 | −6.774 | 21.931 | 1.00 | 42.33 | N |
| ATOM | 849 | CA | LEU | A | 469 | −39.545 | −5.859 | 21.662 | 1.00 | 42.00 | C |
| ATOM | 850 | C | LEU | A | 469 | −40.518 | −6.387 | 20.600 | 1.00 | 41.66 | C |
| ATOM | 851 | O | LEU | A | 469 | −41.730 | −6.341 | 20.800 | 1.00 | 41.78 | O |
| ATOM | 852 | CB | LEU | A | 469 | −39.031 | −4.460 | 21.313 | 1.00 | 41.91 | C |
| ATOM | 853 | CG | LEU | A | 469 | −38.392 | −3.707 | 22.487 | 1.00 | 41.86 | C |
| ATOM | 854 | CD1 | LEU | A | 469 | −37.785 | −2.397 | 22.023 | 1.00 | 41.11 | C |
| ATOM | 855 | CD2 | LEU | A | 469 | −39.394 | −3.476 | 23.627 | 1.00 | 41.72 | C |
| ATOM | 856 | N | VAL | A | 470 | −39.989 | −6.904 | 19.494 | 1.00 | 41.25 | N |
| ATOM | 857 | CA | VAL | A | 470 | −40.817 | −7.511 | 18.453 | 1.00 | 41.04 | C |
| ATOM | 858 | C | VAL | A | 470 | −41.547 | −8.740 | 18.994 | 1.00 | 41.03 | C |
| ATOM | 859 | O | VAL | A | 470 | −42.666 | −9.046 | 18.570 | 1.00 | 40.98 | O |
| ATOM | 860 | CB | VAL | A | 470 | −39.997 | −7.875 | 17.193 | 1.00 | 41.07 | C |
| ATOM | 861 | CG1 | VAL | A | 470 | −40.866 | −8.563 | 16.137 | 1.00 | 40.55 | C |
| ATOM | 862 | CG2 | VAL | A | 470 | −39.364 | −6.631 | 16.602 | 1.00 | 41.17 | C |
| ATOM | 863 | N | GLU | A | 471 | −40.920 | −9.427 | 19.944 | 1.00 | 40.92 | N |
| ATOM | 864 | CA | GLU | A | 471 | −41.544 | −10.579 | 20.586 | 1.00 | 40.87 | C |
| ATOM | 865 | C | GLU | A | 471 | −42.802 | −10.192 | 21.357 | 1.00 | 40.74 | C |
| ATOM | 866 | O | GLU | A | 471 | −43.753 | −10.963 | 21.415 | 1.00 | 40.83 | O |
| ATOM | 867 | CB | GLU | A | 471 | −40.554 | −11.292 | 21.501 | 1.00 | 40.89 | C |
| ATOM | 868 | CG | GLU | A | 471 | −39.552 | −12.176 | 20.767 | 1.00 | 41.35 | C |
| ATOM | 869 | CD | GLU | A | 471 | −38.425 | −12.656 | 21.666 | 1.00 | 42.44 | C |
| ATOM | 870 | OE1 | GLU | A | 471 | −38.581 | −12.614 | 22.905 | 1.00 | 43.05 | O |
| ATOM | 871 | OE2 | GLU | A | 471 | −37.376 | −13.073 | 21.135 | 1.00 | 42.90 | O |
| ATOM | 872 | N | LYS | A | 472 | −42.807 | −8.991 | 21.928 | 1.00 | 40.76 | N |
| ATOM | 873 | CA | LYS | A | 472 | −43.950 | −8.502 | 22.691 | 1.00 | 40.83 | C |
| ATOM | 874 | C | LYS | A | 472 | −45.003 | −7.810 | 21.828 | 1.00 | 40.73 | C |
| ATOM | 875 | O | LYS | A | 472 | −46.194 | −8.084 | 21.974 | 1.00 | 40.70 | O |
| ATOM | 876 | CB | LYS | A | 472 | −43.505 | −7.566 | 23.820 | 1.00 | 41.02 | C |
| ATOM | 877 | CG | LYS | A | 472 | −42.540 | −8.197 | 24.811 | 1.00 | 41.55 | C |
| ATOM | 878 | CD | LYS | A | 472 | −42.745 | −7.680 | 26.237 | 1.00 | 41.89 | C |
| ATOM | 879 | CE | LYS | A | 472 | −41.897 | −8.485 | 27.223 | 1.00 | 42.12 | C |
| ATOM | 880 | NZ | LYS | A | 472 | −42.559 | −8.688 | 28.544 | 1.00 | 42.15 | N |
| ATOM | 881 | N | PHE | A | 473 | −44.573 | −6.925 | 20.931 | 1.00 | 40.68 | N |
| ATOM | 882 | CA | PHE | A | 473 | −45.519 | −6.079 | 20.180 | 1.00 | 40.68 | C |
| ATOM | 883 | C | PHE | A | 473 | −45.984 | −6.591 | 18.808 | 1.00 | 40.71 | C |
| ATOM | 884 | O | PHE | A | 473 | −46.982 | −6.102 | 18.278 | 1.00 | 40.68 | O |
| ATOM | 885 | CB | PHE | A | 473 | −44.996 | −4.646 | 20.053 | 1.00 | 40.51 | C |
| ATOM | 886 | CG | PHE | A | 473 | −44.667 | −3.997 | 21.366 | 1.00 | 40.41 | C |
| ATOM | 887 | CD1 | PHE | A | 473 | −45.502 | −4.151 | 22.468 | 1.00 | 39.58 | C |
| ATOM | 888 | CD2 | PHE | A | 473 | −43.523 | −3.210 | 21.495 | 1.00 | 40.82 | C |
| ATOM | 889 | CE1 | PHE | A | 473 | −45.200 | −3.552 | 23.683 | 1.00 | 39.49 | C |
| ATOM | 890 | CE2 | PHE | A | 473 | −43.212 | −2.598 | 22.710 | 1.00 | 40.83 | C |
| ATOM | 891 | CZ | PHE | A | 473 | −44.057 | −2.775 | 23.808 | 1.00 | 40.24 | C |
| ATOM | 892 | N | GLY | A | 474 | −45.270 | −7.553 | 18.230 | 1.00 | 40.81 | N |
| ATOM | 893 | CA | GLY | A | 474 | −45.686 | −8.148 | 16.961 | 1.00 | 41.03 | C |
| ATOM | 894 | C | GLY | A | 474 | −44.921 | −7.644 | 15.751 | 1.00 | 41.25 | C |
| ATOM | 895 | O | GLY | A | 474 | −44.436 | −6.511 | 15.734 | 1.00 | 41.04 | O |
| ATOM | 896 | N | LYS | A | 475 | −44.829 | −8.499 | 14.733 | 1.00 | 41.58 | N |
| ATOM | 897 | CA | LYS | A | 475 | −44.030 | −8.232 | 13.529 | 1.00 | 41.83 | C |
| ATOM | 898 | C | LYS | A | 475 | −44.560 | −7.045 | 12.723 | 1.00 | 42.11 | C |
| ATOM | 899 | O | LYS | A | 475 | −43.799 | −6.359 | 12.032 | 1.00 | 42.05 | O |
| ATOM | 900 | CB | LYS | A | 475 | −43.916 | −9.497 | 12.657 | 1.00 | 41.78 | C |
| ATOM | 901 | CG | LYS | A | 475 | −45.235 | −10.056 | 12.101 | 1.00 | 41.80 | C |
| ATOM | 902 | CD | LYS | A | 475 | −45.349 | −11.559 | 12.356 | 1.00 | 42.38 | C |
| ATOM | 903 | CE | LYS | A | 475 | −45.951 | −11.835 | 13.746 | 1.00 | 43.35 | C |
| ATOM | 904 | NZ | LYS | A | 475 | −45.315 | −12.978 | 14.475 | 1.00 | 43.15 | N |
| ATOM | 905 | N | GLU | A | 476 | −45.868 | −6.815 | 12.829 | 1.00 | 42.35 | N |
| ATOM | 906 | CA | GLU | A | 476 | −46.532 | −5.700 | 12.164 | 1.00 | 42.48 | C |
| ATOM | 907 | C | GLU | A | 476 | −46.000 | −4.391 | 12.735 | 1.00 | 42.32 | C |
| ATOM | 908 | O | GLU | A | 476 | −45.538 | −3.526 | 11.992 | 1.00 | 42.35 | O |
| ATOM | 909 | CB | GLU | A | 476 | −48.051 | −5.780 | 12.360 | 1.00 | 42.60 | C |
| ATOM | 910 | CG | GLU | A | 476 | −48.578 | −7.155 | 12.802 | 1.00 | 43.40 | C |
| ATOM | 911 | CD | GLU | A | 476 | −48.532 | −8.209 | 11.703 | 1.00 | 44.05 | C |
| ATOM | 912 | OE1 | GLU | A | 476 | −48.471 | −7.835 | 10.510 | 1.00 | 44.58 | O |
| ATOM | 913 | OE2 | GLU | A | 476 | −48.563 | −9.415 | 12.037 | 1.00 | 43.85 | O |
| ATOM | 914 | N | TRP | A | 477 | −46.054 | −4.273 | 14.062 | 1.00 | 42.27 | N |
| ATOM | 915 | CA | TRP | A | 477 | −45.538 | −3.117 | 14.790 | 1.00 | 42.23 | C |
| ATOM | 916 | C | TRP | A | 477 | −44.094 | −2.786 | 14.408 | 1.00 | 42.30 | C |
| ATOM | 917 | O | TRP | A | 477 | −43.722 | −1.616 | 14.304 | 1.00 | 42.32 | O |
| ATOM | 918 | CB | TRP | A | 477 | −45.635 | −3.365 | 16.297 | 1.00 | 42.28 | C |
| ATOM | 919 | CG | TRP | A | 477 | −44.806 | −2.421 | 17.095 | 1.00 | 42.24 | C |
| ATOM | 920 | CD1 | TRP | A | 477 | −45.142 | −1.155 | 17.466 | 1.00 | 42.55 | C |
| ATOM | 921 | CD2 | TRP | A | 477 | −43.488 | −2.653 | 17.602 | 1.00 | 42.60 | C |
| ATOM | 922 | NE1 | TRP | A | 477 | −44.120 | −0.582 | 18.183 | 1.00 | 42.78 | N |
| ATOM | 923 | CE2 | TRP | A | 477 | −43.090 | −1.479 | 18.281 | 1.00 | 42.87 | C |
| ATOM | 924 | CE3 | TRP | A | 477 | −42.602 | −3.740 | 17.550 | 1.00 | 42.78 | C |
| ATOM | 925 | CZ2 | TRP | A | 477 | −41.842 | −1.358 | 18.907 | 1.00 | 42.72 | C |
| ATOM | 926 | CZ3 | TRP | A | 477 | −41.364 | −3.623 | 18.174 | 1.00 | 42.67 | C |
| ATOM | 927 | CH2 | TRP | A | 477 | −40.998 | −2.438 | 18.848 | 1.00 | 42.75 | C |
| ATOM | 928 | N | ALA | A | 478 | −43.294 | −3.830 | 14.210 | 1.00 | 42.33 | N |
| ATOM | 929 | CA | ALA | A | 478 | −41.895 | −3.700 | 13.837 | 1.00 | 42.32 | C |
| ATOM | 930 | C | ALA | A | 478 | −41.717 | −3.092 | 12.447 | 1.00 | 42.48 | C |
| ATOM | 931 | O | ALA | A | 478 | −40.853 | −2.229 | 12.250 | 1.00 | 42.66 | O |
| ATOM | 932 | CB | ALA | A | 478 | −41.217 | −5.051 | 13.912 | 1.00 | 42.37 | C |
| ATOM | 933 | N | HIS | A | 479 | −42.527 | −3.544 | 11.488 | 1.00 | 42.57 | N |
| ATOM | 934 | CA | HIS | A | 479 | −42.458 | −3.041 | 10.112 | 1.00 | 42.63 | C |
| ATOM | 935 | C | HIS | A | 479 | −42.754 | −1.548 | 10.033 | 1.00 | 42.64 | C |
| ATOM | 936 | O | HIS | A | 479 | −42.205 | −0.847 | 9.182 | 1.00 | 42.72 | O |
| ATOM | 937 | CB | HIS | A | 479 | −43.390 | −3.826 | 9.181 | 1.00 | 42.63 | C |
| ATOM | 938 | CG | HIS | A | 479 | −43.654 | −3.143 | 7.874 | 1.00 | 42.86 | C |
| ATOM | 939 | ND1 | HIS | A | 479 | −42.647 | −2.622 | 7.088 | 1.00 | 42.92 | N |
| ATOM | 940 | CD2 | HIS | A | 479 | −44.812 | −2.891 | 7.218 | 1.00 | 43.05 | C |
| ATOM | 941 | CE1 | HIS | A | 479 | −43.175 | −2.071 | 6.009 | 1.00 | 43.25 | C |
| ATOM | 942 | NE2 | HIS | A | 479 | −44.486 | −2.227 | 6.060 | 1.00 | 43.23 | N |
| ATOM | 943 | N | ALA | A | 480 | −43.619 | −1.071 | 10.921 | 1.00 | 42.67 | N |
| ATOM | 944 | CA | ALA | A | 480 | −43.940 | 0.344 | 10.998 | 1.00 | 42.87 | C |
| ATOM | 945 | C | ALA | A | 480 | −42.832 | 1.114 | 11.711 | 1.00 | 43.08 | C |
| ATOM | 946 | O | ALA | A | 480 | −42.108 | 1.893 | 11.095 | 1.00 | 43.15 | O |
| ATOM | 947 | CB | ALA | A | 480 | −45.282 | 0.549 | 11.702 | 1.00 | 42.84 | C |
| ATOM | 948 | N | THR | A | 481 | −42.696 | 0.863 | 13.008 | 1.00 | 43.46 | N |
| ATOM | 949 | CA | THR | A | 481 | −41.848 | 1.658 | 13.887 | 1.00 | 43.74 | C |
| ATOM | 950 | C | THR | A | 481 | −40.351 | 1.392 | 13.735 | 1.00 | 43.83 | C |
| ATOM | 951 | O | THR | A | 481 | −39.582 | 2.305 | 13.445 | 1.00 | 44.15 | O |
| ATOM | 952 | CB | THR | A | 481 | −42.257 | 1.442 | 15.360 | 1.00 | 43.76 | C |
| ATOM | 953 | OG1 | THR | A | 481 | −43.644 | 1.756 | 15.511 | 1.00 | 43.77 | O |
| ATOM | 954 | CG2 | THR | A | 481 | −41.423 | 2.320 | 16.302 | 1.00 | 43.92 | C |
| ATOM | 955 | N | ILE | A | 482 | −39.950 | 0.143 | 13.929 | 1.00 | 43.93 | N |
| ATOM | 956 | CA | ILE | A | 482 | −38.550 | −0.190 | 14.160 | 1.00 | 44.07 | C |
| ATOM | 957 | C | ILE | A | 482 | −37.718 | −0.245 | 12.884 | 1.00 | 44.28 | C |
| ATOM | 958 | O | ILE | A | 482 | −36.704 | 0.457 | 12.765 | 1.00 | 44.34 | O |
| ATOM | 959 | CB | ILE | A | 482 | −38.413 | −1.531 | 14.949 | 1.00 | 44.05 | C |
| ATOM | 960 | CG1 | ILE | A | 482 | −39.185 | −1.470 | 16.272 | 1.00 | 43.69 | C |
| ATOM | 961 | CG2 | ILE | A | 482 | −36.943 | −1.900 | 15.172 | 1.00 | 44.09 | C |
| ATOM | 962 | CD1 | ILE | A | 482 | −38.798 | −0.313 | 17.193 | 1.00 | 43.61 | C |
| ATOM | 963 | N | ILE | A | 483 | −38.159 | −1.070 | 11.938 | 1.00 | 44.41 | N |
| ATOM | 964 | CA | ILE | A | 483 | −37.361 | −1.399 | 10.757 | 1.00 | 44.75 | C |
| ATOM | 965 | C | ILE | A | 483 | −36.801 | −0.186 | 9.984 | 1.00 | 45.03 | C |
| ATOM | 966 | O | ILE | A | 483 | −35.629 | −0.204 | 9.608 | 1.00 | 45.11 | O |
| ATOM | 967 | CB | ILE | A | 483 | −38.091 | −2.418 | 9.837 | 1.00 | 44.75 | C |
| ATOM | 968 | CG1 | ILE | A | 483 | −38.139 | −3.786 | 10.527 | 1.00 | 44.79 | C |
| ATOM | 969 | CG2 | ILE | A | 483 | −37.403 | −2.523 | 8.469 | 1.00 | 44.59 | C |
| ATOM | 970 | CD1 | ILE | A | 483 | −39.130 | −4.762 | 9.922 | 1.00 | 46.13 | C |
| ATOM | 971 | N | PRO | A | 484 | −37.618 | 0.866 | 9.754 | 1.00 | 45.30 | N |
| ATOM | 972 | CA | PRO | A | 484 | −37.051 | 2.011 | 9.035 | 1.00 | 45.65 | C |
| ATOM | 973 | C | PRO | A | 484 | −35.856 | 2.668 | 9.746 | 1.00 | 45.88 | C |
| ATOM | 974 | O | PRO | A | 484 | −34.844 | 2.956 | 9.104 | 1.00 | 45.86 | O |
| ATOM | 975 | CB | PRO | A | 484 | −38.236 | 2.984 | 8.932 | 1.00 | 45.63 | C |
| ATOM | 976 | CG | PRO | A | 484 | −39.442 | 2.133 | 9.095 | 1.00 | 45.30 | C |
| ATOM | 977 | CD | PRO | A | 484 | −39.040 | 1.087 | 10.074 | 1.00 | 45.27 | C |
| ATOM | 978 | N | LYS | A | 485 | −35.968 | 2.881 | 11.054 | 1.00 | 46.15 | N |
| ATOM | 979 | CA | LYS | A | 485 | −34.902 | 3.522 | 11.831 | 1.00 | 46.65 | C |
| ATOM | 980 | C | LYS | A | 485 | −33.622 | 2.682 | 11.839 | 1.00 | 46.67 | C |
| ATOM | 981 | O | LYS | A | 485 | −32.510 | 3.216 | 11.928 | 1.00 | 46.64 | O |
| ATOM | 982 | CB | LYS | A | 485 | −35.383 | 3.807 | 13.254 | 1.00 | 46.85 | C |
| ATOM | 983 | CG | LYS | A | 485 | −36.493 | 4.853 | 13.303 | 1.00 | 47.66 | C |
| ATOM | 984 | CD | LYS | A | 485 | −37.406 | 4.697 | 14.519 | 1.00 | 48.44 | C |
| ATOM | 985 | CE | LYS | A | 485 | −38.675 | 5.524 | 14.350 | 1.00 | 49.10 | C |
| ATOM | 986 | NZ | LYS | A | 485 | −38.394 | 6.883 | 13.777 | 1.00 | 49.74 | N |
| ATOM | 987 | N | VAL | A | 486 | −33.806 | 1.367 | 11.732 | 1.00 | 46.62 | N |
| ATOM | 988 | CA | VAL | A | 486 | −32.716 | 0.412 | 11.549 | 1.00 | 46.38 | C |
| ATOM | 989 | C | VAL | A | 486 | −32.113 | 0.494 | 10.129 | 1.00 | 46.33 | C |
| ATOM | 990 | O | VAL | A | 486 | −30.900 | 0.330 | 9.952 | 1.00 | 46.35 | O |
| ATOM | 991 | CB | VAL | A | 486 | −33.193 | −1.027 | 11.865 | 1.00 | 46.20 | C |
| ATOM | 992 | CG1 | VAL | A | 486 | −32.119 | −2.044 | 11.535 | 1.00 | 46.27 | C |
| ATOM | 993 | CG2 | VAL | A | 486 | −33.597 | −1.141 | 13.327 | 1.00 | 46.11 | C |
| ATOM | 994 | N | LEU | A | 487 | −32.949 | 0.757 | 9.128 | 1.00 | 46.20 | N |
| ATOM | 995 | CA | LEU | A | 487 | −32.467 | 0.887 | 7.751 | 1.00 | 46.26 | C |
| ATOM | 996 | C | LEU | A | 487 | −31.659 | 2.166 | 7.524 | 1.00 | 46.25 | C |
| ATOM | 997 | O | LEU | A | 487 | −30.840 | 2.230 | 6.611 | 1.00 | 46.15 | O |
| ATOM | 998 | CB | LEU | A | 487 | −33.621 | 0.799 | 6.745 | 1.00 | 46.22 | C |
| ATOM | 999 | CG | LEU | A | 487 | −34.363 | −0.531 | 6.571 | 1.00 | 46.11 | C |
| ATOM | 1000 | CD1 | LEU | A | 487 | −35.216 | −0.463 | 5.318 | 1.00 | 46.68 | C |
| ATOM | 1001 | CD2 | LEU | A | 487 | −33.424 | −1.733 | 6.505 | 1.00 | 45.69 | C |
| ATOM | 1002 | N | ALA | A | 488 | −31.901 | 3.173 | 8.360 | 1.00 | 46.37 | N |
| ATOM | 1003 | CA | ALA | A | 488 | −31.173 | 4.438 | 8.314 | 1.00 | 46.47 | C |
| ATOM | 1004 | C | ALA | A | 488 | −29.666 | 4.248 | 8.488 | 1.00 | 46.64 | C |
| ATOM | 1005 | O | ALA | A | 488 | −28.865 | 4.761 | 7.701 | 1.00 | 46.75 | O |
| ATOM | 1006 | CB | ALA | A | 488 | −31.711 | 5.389 | 9.371 | 1.00 | 46.46 | C |
| ATOM | 1007 | N | MET | A | 489 | −29.280 | 3.494 | 9.510 | 1.00 | 46.76 | N |
| ATOM | 1008 | CA | MET | A | 489 | −27.867 | 3.306 | 9.826 | 1.00 | 46.88 | C |
| ATOM | 1009 | C | MET | A | 489 | −27.061 | 2.656 | 8.696 | 1.00 | 46.79 | C |
| ATOM | 1010 | O | MET | A | 489 | −25.830 | 2.628 | 8.756 | 1.00 | 47.09 | O |
| ATOM | 1011 | CB | MET | A | 489 | −27.696 | 2.505 | 11.115 | 1.00 | 46.80 | C |
| ATOM | 1012 | CG | MET | A | 489 | −28.394 | 3.080 | 12.331 | 1.00 | 46.86 | C |
| ATOM | 1013 | SD | MET | A | 489 | −27.974 | 2.119 | 13.796 | 1.00 | 47.22 | S |
| ATOM | 1014 | CE | MET | A | 489 | −28.646 | 0.506 | 13.371 | 1.00 | 46.32 | C |
| ATOM | 1015 | N | SER | A | 490 | −27.745 | 2.138 | 7.676 | 1.00 | 46.56 | N |
| ATOM | 1016 | CA | SER | A | 490 | −27.069 | 1.542 | 6.517 | 1.00 | 46.30 | C |
| ATOM | 1017 | C | SER | A | 490 | −26.261 | 2.577 | 5.726 | 1.00 | 46.13 | C |
| ATOM | 1018 | O | SER | A | 490 | −25.243 | 2.244 | 5.128 | 1.00 | 46.02 | O |
| ATOM | 1019 | CB | SER | A | 490 | −28.069 | 0.847 | 5.594 | 1.00 | 46.25 | C |
| ATOM | 1020 | OG | SER | A | 490 | −28.835 | 1.794 | 4.864 | 1.00 | 46.28 | O |
| ATOM | 1021 | N | GLY | A | 491 | −26.724 | 3.824 | 5.724 | 1.00 | 45.96 | N |
| ATOM | 1022 | CA | GLY | A | 491 | −26.028 | 4.897 | 5.024 | 1.00 | 45.83 | C |
| ATOM | 1023 | C | GLY | A | 491 | −25.039 | 5.661 | 5.881 | 1.00 | 45.76 | C |
| ATOM | 1024 | O | GLY | A | 491 | −24.342 | 6.543 | 5.385 | 1.00 | 45.71 | O |
| ATOM | 1025 | N | ASP | A | 492 | −24.984 | 5.319 | 7.167 | 1.00 | 45.91 | N |
| ATOM | 1026 | CA | ASP | A | 492 | −24.099 | 5.974 | 8.133 | 1.00 | 46.10 | C |
| ATOM | 1027 | C | ASP | A | 492 | −22.631 | 5.945 | 7.696 | 1.00 | 46.08 | C |
| ATOM | 1028 | O | ASP | A | 492 | −22.086 | 4.867 | 7.416 | 1.00 | 46.12 | O |
| ATOM | 1029 | CB | ASP | A | 492 | −24.236 | 5.307 | 9.502 | 1.00 | 46.18 | C |
| ATOM | 1030 | CG | ASP | A | 492 | −23.642 | 6.137 | 10.632 | 1.00 | 46.97 | C |
| ATOM | 1031 | OD1 | ASP | A | 492 | −24.371 | 6.361 | 11.626 | 1.00 | 47.76 | O |
| ATOM | 1032 | OD2 | ASP | A | 492 | −22.457 | 6.545 | 10.551 | 1.00 | 47.36 | O |
| ATOM | 1033 | N | PRO | A | 493 | −21.987 | 7.132 | 7.647 | 1.00 | 45.98 | N |
| ATOM | 1034 | CA | PRO | A | 493 | −20.567 | 7.277 | 7.299 | 1.00 | 45.83 | C |
| ATOM | 1035 | C | PRO | A | 493 | −19.630 | 6.343 | 8.078 | 1.00 | 45.65 | C |
| ATOM | 1036 | O | PRO | A | 493 | −18.676 | 5.820 | 7.495 | 1.00 | 45.59 | O |
| ATOM | 1037 | CB | PRO | A | 493 | −20.262 | 8.749 | 7.634 | 1.00 | 45.72 | C |
| ATOM | 1038 | CG | PRO | A | 493 | −21.457 | 9.257 | 8.393 | 1.00 | 45.73 | C |
| ATOM | 1039 | CD | PRO | A | 493 | −22.608 | 8.441 | 7.918 | 1.00 | 46.03 | C |
| ATOM | 1040 | N | ASN | A | 494 | −19.905 | 6.136 | 9.369 | 1.00 | 45.38 | N |
| ATOM | 1041 | CA | ASN | A | 494 | −19.068 | 5.286 | 10.213 | 1.00 | 45.08 | C |
| ATOM | 1042 | C | ASN | A | 494 | −19.411 | 3.817 | 10.044 | 1.00 | 45.20 | C |
| ATOM | 1043 | O | ASN | A | 494 | −20.564 | 3.417 | 10.234 | 1.00 | 45.61 | O |
| ATOM | 1044 | CB | ASN | A | 494 | −19.187 | 5.689 | 11.684 | 1.00 | 44.96 | C |
| ATOM | 1045 | CG | ASN | A | 494 | −18.117 | 5.048 | 12.557 | 1.00 | 44.56 | C |
| ATOM | 1046 | OD1 | ASN | A | 494 | −17.920 | 3.835 | 12.534 | 1.00 | 44.75 | O |
| ATOM | 1047 | ND2 | ASN | A | 494 | −17.428 | 5.864 | 13.337 | 1.00 | 44.29 | N |
| ATOM | 1048 | N | TYR | A | 495 | −18.403 | 3.013 | 9.711 | 1.00 | 44.98 | N |
| ATOM | 1049 | CA | TYR | A | 495 | −18.603 | 1.583 | 9.457 | 1.00 | 45.04 | C |
| ATOM | 1050 | C | TYR | A | 495 | −19.063 | 0.789 | 10.690 | 1.00 | 45.11 | C |
| ATOM | 1051 | O | TYR | A | 495 | −19.708 | −0.255 | 10.553 | 1.00 | 44.99 | O |
| ATOM | 1052 | CB | TYR | A | 495 | −17.347 | 0.953 | 8.828 | 1.00 | 45.02 | C |
| ATOM | 1053 | CG | TYR | A | 495 | −16.300 | 0.472 | 9.817 | 1.00 | 44.94 | C |
| ATOM | 1054 | CD1 | TYR | A | 495 | −15.742 | 1.342 | 10.757 | 1.00 | 45.07 | C |
| ATOM | 1055 | CD2 | TYR | A | 495 | −15.863 | −0.853 | 9.806 | 1.00 | 44.75 | C |
| ATOM | 1056 | CE1 | TYR | A | 495 | −14.786 | 0.906 | 11.663 | 1.00 | 45.18 | C |
| ATOM | 1057 | CE2 | TYR | A | 495 | −14.902 | −1.301 | 10.711 | 1.00 | 44.87 | C |
| ATOM | 1058 | CZ | TYR | A | 495 | −14.363 | −0.410 | 11.630 | 1.00 | 44.92 | C |
| ATOM | 1059 | OH | TYR | A | 495 | −13.412 | −0.826 | 12.528 | 1.00 | 44.90 | O |
| ATOM | 1060 | N | LEU | A | 496 | −18.729 | 1.277 | 11.885 | 1.00 | 45.12 | N |
| ATOM | 1061 | CA | LEU | A | 496 | −19.176 | 0.628 | 13.114 | 1.00 | 45.34 | C |
| ATOM | 1062 | C | LEU | A | 496 | −20.694 | 0.676 | 13.213 | 1.00 | 45.45 | C |
| ATOM | 1063 | O | LEU | A | 496 | −21.309 | −0.202 | 13.817 | 1.00 | 45.52 | O |
| ATOM | 1064 | CB | LEU | A | 496 | −18.555 | 1.269 | 14.357 | 1.00 | 45.35 | C |
| ATOM | 1065 | CG | LEU | A | 496 | −17.045 | 1.136 | 14.596 | 1.00 | 45.37 | C |
| ATOM | 1066 | CD1 | LEU | A | 496 | −16.593 | 2.111 | 15.690 | 1.00 | 44.01 | C |
| ATOM | 1067 | CD2 | LEU | A | 496 | −16.640 | −0.302 | 14.934 | 1.00 | 44.67 | C |
| ATOM | 1068 | N | HIS | A | 497 | −21.288 | 1.700 | 12.604 | 1.00 | 45.35 | N |
| ATOM | 1069 | CA | HIS | A | 497 | −22.740 | 1.852 | 12.594 | 1.00 | 45.27 | C |
| ATOM | 1070 | C | HIS | A | 497 | −23.394 | 1.067 | 11.461 | 1.00 | 44.81 | C |
| ATOM | 1071 | O | HIS | A | 497 | −24.407 | 0.405 | 11.655 | 1.00 | 44.65 | O |
| ATOM | 1072 | CB | HIS | A | 497 | −23.115 | 3.331 | 12.510 | 1.00 | 45.45 | C |
| ATOM | 1073 | CG | HIS | A | 497 | −22.762 | 4.110 | 13.736 | 1.00 | 45.56 | C |
| ATOM | 1074 | ND1 | HIS | A | 497 | −22.667 | 5.483 | 13.741 | 1.00 | 45.77 | N |
| ATOM | 1075 | CD2 | HIS | A | 497 | −22.479 | 3.708 | 14.998 | 1.00 | 45.90 | C |
| ATOM | 1076 | CE1 | HIS | A | 497 | −22.340 | 5.895 | 14.953 | 1.00 | 46.39 | C |
| ATOM | 1077 | NE2 | HIS | A | 497 | −22.223 | 4.838 | 15.736 | 1.00 | 46.20 | N |
| ATOM | 1078 | N | ARG | A | 498 | −22.797 | 1.161 | 10.279 | 1.00 | 44.65 | N |
| ATOM | 1079 | CA | ARG | A | 498 | −23.219 | 0.413 | 9.098 | 1.00 | 44.07 | C |
| ATOM | 1080 | C | ARG | A | 498 | −23.357 | −1.084 | 9.431 | 1.00 | 44.00 | C |
| ATOM | 1081 | O | ARG | A | 498 | −24.295 | −1.743 | 8.981 | 1.00 | 43.98 | O |
| ATOM | 1082 | CB | ARG | A | 498 | −22.211 | 0.655 | 7.966 | 1.00 | 43.79 | C |
| ATOM | 1083 | CG | ARG | A | 498 | −22.825 | 0.811 | 6.589 | 1.00 | 43.41 | C |
| ATOM | 1084 | CD | ARG | A | 498 | −22.203 | 1.962 | 5.768 | 1.00 | 41.35 | C |
| ATOM | 1085 | NE | ARG | A | 498 | −20.823 | 1.704 | 5.369 | 1.00 | 39.95 | N |
| ATOM | 1086 | CZ | ARG | A | 498 | −19.766 | 2.381 | 5.816 | 1.00 | 39.80 | C |
| ATOM | 1087 | NH1 | ARG | A | 498 | −19.912 | 3.385 | 6.675 | 1.00 | 39.45 | N |
| ATOM | 1088 | NH2 | ARG | A | 498 | −18.553 | 2.053 | 5.397 | 1.00 | 39.61 | N |
| ATOM | 1089 | N | MET | A | 499 | −22.438 | −1.600 | 10.250 | 1.00 | 43.81 | N |
| ATOM | 1090 | CA | MET | A | 499 | −22.478 | −3.001 | 10.689 | 1.00 | 43.54 | C |
| ATOM | 1091 | C | MET | A | 499 | −23.583 | −3.286 | 11.718 | 1.00 | 43.40 | C |
| ATOM | 1092 | O | MET | A | 499 | −24.125 | −4.387 | 11.743 | 1.00 | 43.39 | O |
| ATOM | 1093 | CB | MET | A | 499 | −21.117 | −3.452 | 11.231 | 1.00 | 43.46 | C |
| ATOM | 1094 | CG | MET | A | 499 | −20.026 | −3.591 | 10.170 | 1.00 | 43.32 | C |
| ATOM | 1095 | SD | MET | A | 499 | −18.479 | −4.274 | 10.812 | 1.00 | 43.29 | S |
| ATOM | 1096 | CE | MET | A | 499 | −18.095 | −3.127 | 12.128 | 1.00 | 42.88 | C |
| ATOM | 1097 | N | THR | A | 500 | −23.908 | −2.296 | 12.553 | 1.00 | 43.06 | N |
| ATOM | 1098 | CA | THR | A | 500 | −25.012 | −2.402 | 13.509 | 1.00 | 42.68 | C |
| ATOM | 1099 | C | THR | A | 500 | −26.318 | −2.703 | 12.774 | 1.00 | 42.90 | C |
| ATOM | 1100 | O | THR | A | 500 | −27.148 | −3.482 | 13.259 | 1.00 | 43.15 | O |
| ATOM | 1101 | CB | THR | A | 500 | −25.176 | −1.114 | 14.343 | 1.00 | 42.49 | C |
| ATOM | 1102 | OG1 | THR | A | 500 | −23.916 | −0.740 | 14.907 | 1.00 | 42.51 | O |
| ATOM | 1103 | CG2 | THR | A | 500 | −26.163 | −1.318 | 15.468 | 1.00 | 42.28 | C |
| ATOM | 1104 | N | THR | A | 501 | −26.491 | −2.091 | 11.602 | 1.00 | 42.62 | N |
| ATOM | 1105 | CA | THR | A | 501 | −27.635 | −2.379 | 10.743 | 1.00 | 42.50 | C |
| ATOM | 1106 | C | THR | A | 501 | −27.711 | −3.873 | 10.433 | 1.00 | 42.31 | C |
| ATOM | 1107 | O | THR | A | 501 | −28.779 | −4.477 | 10.510 | 1.00 | 42.31 | O |
| ATOM | 1108 | CB | THR | A | 501 | −27.573 | −1.557 | 9.433 | 1.00 | 42.59 | C |
| ATOM | 1109 | OG1 | THR | A | 501 | −27.935 | −0.202 | 9.712 | 1.00 | 42.88 | O |
| ATOM | 1110 | CG2 | THR | A | 501 | −28.524 | −2.114 | 8.366 | 1.00 | 42.42 | C |
| ATOM | 1111 | N | LEU | A | 502 | −26.566 | −4.459 | 10.101 | 1.00 | 42.16 | N |
| ATOM | 1112 | CA | LEU | A | 502 | −26.501 | −5.856 | 9.710 | 1.00 | 42.02 | C |
| ATOM | 1113 | C | LEU | A | 502 | −26.700 | −6.786 | 10.893 | 1.00 | 42.03 | C |
| ATOM | 1114 | O | LEU | A | 502 | −27.182 | −7.902 | 10.729 | 1.00 | 41.99 | O |
| ATOM | 1115 | CB | LEU | A | 502 | −25.182 | −6.150 | 8.995 | 1.00 | 42.05 | C |
| ATOM | 1116 | CG | LEU | A | 502 | −25.093 | −5.616 | 7.555 | 1.00 | 42.18 | C |
| ATOM | 1117 | CD1 | LEU | A | 502 | −23.654 | −5.653 | 7.026 | 1.00 | 42.01 | C |
| ATOM | 1118 | CD2 | LEU | A | 502 | −26.035 | −6.373 | 6.617 | 1.00 | 41.55 | C |
| ATOM | 1119 | N | PHE | A | 503 | −26.349 | −6.313 | 12.086 | 1.00 | 42.07 | N |
| ATOM | 1120 | CA | PHE | A | 503 | −26.538 | −7.092 | 13.302 | 1.00 | 42.17 | C |
| ATOM | 1121 | C | PHE | A | 503 | −28.014 | −7.089 | 13.708 | 1.00 | 42.47 | C |
| ATOM | 1122 | O | PHE | A | 503 | −28.524 | −8.077 | 14.259 | 1.00 | 42.39 | O |
| ATOM | 1123 | CB | PHE | A | 503 | −25.650 | −6.577 | 14.448 | 1.00 | 41.94 | C |
| ATOM | 1124 | CG | PHE | A | 503 | −24.169 | −6.580 | 14.137 | 1.00 | 41.53 | C |
| ATOM | 1125 | CD1 | PHE | A | 503 | −23.603 | −7.559 | 13.316 | 1.00 | 40.46 | C |
| ATOM | 1126 | CD2 | PHE | A | 503 | −23.333 | −5.604 | 14.685 | 1.00 | 42.42 | C |
| ATOM | 1127 | CE1 | PHE | A | 503 | −22.237 | −7.566 | 13.029 | 1.00 | 39.85 | C |
| ATOM | 1128 | CE2 | PHE | A | 503 | −21.953 | −5.596 | 14.407 | 1.00 | 41.90 | C |
| ATOM | 1129 | CZ | PHE | A | 503 | −21.408 | −6.587 | 13.578 | 1.00 | 41.52 | C |
| ATOM | 1130 | N | CYS | A | 504 | −28.695 | −5.985 | 13.415 | 1.00 | 42.70 | N |
| ATOM | 1131 | CA | CYS | A | 504 | −30.123 | −5.864 | 13.694 | 1.00 | 43.19 | C |
| ATOM | 1132 | C | CYS | A | 504 | −30.952 | −6.731 | 12.766 | 1.00 | 43.28 | C |
| ATOM | 1133 | O | CYS | A | 504 | −31.882 | −7.410 | 13.210 | 1.00 | 43.22 | O |
| ATOM | 1134 | CB | CYS | A | 504 | −30.567 | −4.416 | 13.588 | 1.00 | 43.15 | C |
| ATOM | 1135 | SG | CYS | A | 504 | −29.893 | −3.393 | 14.878 | 1.00 | 44.32 | S |
| ATOM | 1136 | N | ILE | A | 505 | −30.602 | −6.705 | 11.481 | 1.00 | 43.52 | N |
| ATOM | 1137 | CA | ILE | A | 505 | −31.237 | −7.554 | 10.479 | 1.00 | 43.68 | C |
| ATOM | 1138 | C | ILE | A | 505 | −31.012 | −9.034 | 10.819 | 1.00 | 43.93 | C |
| ATOM | 1139 | O | ILE | A | 505 | −31.932 | −9.846 | 10.676 | 1.00 | 44.00 | O |
| ATOM | 1140 | CB | ILE | A | 505 | −30.757 | −7.201 | 9.052 | 1.00 | 43.79 | C |
| ATOM | 1141 | CG1 | ILE | A | 505 | −31.253 | −5.802 | 8.671 | 1.00 | 43.79 | C |
| ATOM | 1142 | CG2 | ILE | A | 505 | −31.235 | −8.243 | 8.031 | 1.00 | 43.58 | C |
| ATOM | 1143 | CD1 | ILE | A | 505 | −30.497 | −5.151 | 7.532 | 1.00 | 43.53 | C |
| ATOM | 1144 | N | ASN | A | 506 | −29.809 | −9.371 | 11.294 | 1.00 | 44.02 | N |
| ATOM | 1145 | CA | ASN | A | 506 | −29.538 | −10.715 | 11.830 | 1.00 | 44.27 | C |
| ATOM | 1146 | C | ASN | A | 506 | −30.514 | −11.118 | 12.944 | 1.00 | 44.44 | C |
| ATOM | 1147 | O | ASN | A | 506 | −31.040 | −12.236 | 12.946 | 1.00 | 44.68 | O |
| ATOM | 1148 | CB | ASN | A | 506 | −28.106 | −10.834 | 12.362 | 1.00 | 44.31 | C |
| ATOM | 1149 | CG | ASN | A | 506 | −27.041 | −10.709 | 11.274 | 1.00 | 44.29 | C |
| ATOM | 1150 | OD1 | ASN | A | 506 | −25.879 | −10.440 | 11.578 | 1.00 | 44.97 | O |
| ATOM | 1151 | ND2 | ASN | A | 506 | −27.426 | −10.896 | 10.017 | 1.00 | 43.39 | N |
| ATOM | 1152 | N | VAL | A | 507 | −30.746 | −10.199 | 13.881 | 1.00 | 44.29 | N |
| ATOM | 1153 | CA | VAL | A | 507 | −31.640 | −10.428 | 15.012 | 1.00 | 44.11 | C |
| ATOM | 1154 | C | VAL | A | 507 | −33.115 | −10.392 | 14.594 | 1.00 | 44.31 | C |
| ATOM | 1155 | O | VAL | A | 507 | −33.911 | −11.236 | 15.029 | 1.00 | 44.32 | O |
| ATOM | 1156 | CB | VAL | A | 507 | −31.348 | −9.410 | 16.154 | 1.00 | 44.06 | C |
| ATOM | 1157 | CG1 | VAL | A | 507 | −32.603 | −9.016 | 16.902 | 1.00 | 43.85 | C |
| ATOM | 1158 | CG2 | VAL | A | 507 | −30.303 | −9.960 | 17.104 | 1.00 | 43.66 | C |
| ATOM | 1159 | N | LEU | A | 508 | −33.471 | −9.430 | 13.743 | 1.00 | 44.32 | N |
| ATOM | 1160 | CA | LEU | A | 508 | −34.874 | −9.220 | 13.375 | 1.00 | 44.44 | C |
| ATOM | 1161 | C | LEU | A | 508 | −35.445 | −10.315 | 12.474 | 1.00 | 44.59 | C |
| ATOM | 1162 | O | LEU | A | 508 | −36.604 | −10.691 | 12.612 | 1.00 | 44.54 | O |
| ATOM | 1163 | CB | LEU | A | 508 | −35.082 | −7.835 | 12.764 | 1.00 | 44.18 | C |
| ATOM | 1164 | CG | LEU | A | 508 | −34.891 | −6.689 | 13.763 | 1.00 | 44.03 | C |
| ATOM | 1165 | CD1 | LEU | A | 508 | −34.709 | −5.364 | 13.042 | 1.00 | 44.08 | C |
| ATOM | 1166 | CD2 | LEU | A | 508 | −36.030 | −6.601 | 14.775 | 1.00 | 43.61 | C |
| ATOM | 1167 | N | SER | A | 509 | −34.616 | −10.833 | 11.574 | 1.00 | 44.95 | N |
| ATOM | 1168 | CA | SER | A | 509 | −34.992 | −11.943 | 10.699 | 1.00 | 45.31 | C |
| ATOM | 1169 | C | SER | A | 509 | −35.551 | −13.139 | 11.462 | 1.00 | 45.43 | C |
| ATOM | 1170 | O | SER | A | 509 | −36.315 | −13.930 | 10.906 | 1.00 | 45.45 | O |
| ATOM | 1171 | CB | SER | A | 509 | −33.780 | −12.407 | 9.891 | 1.00 | 45.44 | C |
| ATOM | 1172 | OG | SER | A | 509 | −32.831 | −13.061 | 10.726 | 1.00 | 45.72 | O |
| ATOM | 1173 | N | GLU | A | 510 | −35.156 | −13.272 | 12.727 | 1.00 | 45.60 | N |
| ATOM | 1174 | CA | GLU | A | 510 | −35.560 | −14.410 | 13.549 | 1.00 | 45.89 | C |
| ATOM | 1175 | C | GLU | A | 510 | −37.022 | −14.340 | 13.964 | 1.00 | 45.57 | C |
| ATOM | 1176 | O | GLU | A | 510 | −37.670 | −15.364 | 14.113 | 1.00 | 45.63 | O |
| ATOM | 1177 | CB | GLU | A | 510 | −34.691 | −14.507 | 14.802 | 1.00 | 45.94 | C |
| ATOM | 1178 | CG | GLU | A | 510 | −33.227 | −14.840 | 14.555 | 1.00 | 46.44 | C |
| ATOM | 1179 | CD | GLU | A | 510 | −32.444 | −14.959 | 15.851 | 1.00 | 46.70 | C |
| ATOM | 1180 | OE1 | GLU | A | 510 | −33.007 | −15.483 | 16.838 | 1.00 | 47.97 | O |
| ATOM | 1181 | OE2 | GLU | A | 510 | −31.268 | −14.529 | 15.888 | 1.00 | 47.87 | O |
| ATOM | 1182 | N | VAL | A | 511 | −37.537 | −13.131 | 14.153 | 1.00 | 45.48 | N |
| ATOM | 1183 | CA | VAL | A | 511 | −38.882 | −12.962 | 14.683 | 1.00 | 45.46 | C |
| ATOM | 1184 | C | VAL | A | 511 | −39.813 | −12.190 | 13.752 | 1.00 | 45.62 | C |
| ATOM | 1185 | O | VAL | A | 511 | −40.943 | −11.884 | 14.128 | 1.00 | 45.77 | O |
| ATOM | 1186 | CB | VAL | A | 511 | −38.875 | −12.321 | 16.103 | 1.00 | 45.42 | C |
| ATOM | 1187 | CG1 | VAL | A | 511 | −38.281 | −13.280 | 17.116 | 1.00 | 45.25 | C |
| ATOM | 1188 | CG2 | VAL | A | 511 | −38.117 | −11.001 | 16.114 | 1.00 | 45.51 | C |
| ATOM | 1189 | N | CYS | A | 512 | −39.352 | −11.893 | 12.540 | 1.00 | 45.73 | N |
| ATOM | 1190 | CA | CYS | A | 512 | −40.188 | −11.188 | 11.559 | 1.00 | 45.90 | C |
| ATOM | 1191 | C | CYS | A | 512 | −40.849 | −12.114 | 10.535 | 1.00 | 45.98 | C |
| ATOM | 1192 | O | CYS | A | 512 | −41.870 | −11.758 | 9.942 | 1.00 | 46.04 | O |
| ATOM | 1193 | CB | CYS | A | 512 | −39.396 | −10.091 | 10.844 | 1.00 | 46.02 | C |
| ATOM | 1194 | SG | CYS | A | 512 | −39.016 | −8.648 | 11.869 | 1.00 | 46.07 | S |
| ATOM | 1195 | N | GLY | A | 513 | −40.268 | −13.294 | 10.333 | 1.00 | 46.01 | N |
| ATOM | 1196 | CA | GLY | A | 513 | −40.812 | −14.270 | 9.387 | 1.00 | 46.05 | C |
| ATOM | 1197 | C | GLY | A | 513 | −40.345 | −14.054 | 7.957 | 1.00 | 46.08 | C |
| ATOM | 1198 | O | GLY | A | 513 | −39.585 | −13.125 | 7.675 | 1.00 | 45.97 | O |
| ATOM | 1199 | N | GLN | A | 514 | −40.820 | −14.910 | 7.055 | 1.00 | 46.12 | N |
| ATOM | 1200 | CA | GLN | A | 514 | −40.382 | −14.917 | 5.658 | 1.00 | 46.18 | C |
| ATOM | 1201 | C | GLN | A | 514 | −40.723 | −13.637 | 4.879 | 1.00 | 46.22 | C |
| ATOM | 1202 | O | GLN | A | 514 | −39.837 | −13.005 | 4.298 | 1.00 | 46.12 | O |
| ATOM | 1203 | CB | GLN | A | 514 | −40.962 | −16.132 | 4.936 | 1.00 | 46.14 | C |
| ATOM | 1204 | CG | GLN | A | 514 | −40.250 | −16.466 | 3.646 | 1.00 | 46.26 | C |
| ATOM | 1205 | CD | GLN | A | 514 | −41.070 | −17.355 | 2.750 | 1.00 | 46.22 | C |
| ATOM | 1206 | OE1 | GLN | A | 514 | −42.097 | −16.940 | 2.213 | 1.00 | 46.27 | O |
| ATOM | 1207 | NE2 | GLN | A | 514 | −40.617 | −18.589 | 2.572 | 1.00 | 46.35 | N |
| ATOM | 1208 | N | ASP | A | 515 | −42.003 | −13.270 | 4.873 | 1.00 | 46.30 | N |
| ATOM | 1209 | CA | ASP | A | 515 | −42.509 | −12.173 | 4.042 | 1.00 | 46.36 | C |
| ATOM | 1210 | C | ASP | A | 515 | −41.885 | −10.807 | 4.362 | 1.00 | 46.28 | C |
| ATOM | 1211 | O | ASP | A | 515 | −41.437 | −10.105 | 3.455 | 1.00 | 46.24 | O |
| ATOM | 1212 | CB | ASP | A | 515 | −44.042 | −12.106 | 4.130 | 1.00 | 46.40 | C |
| ATOM | 1213 | CG | ASP | A | 515 | −44.679 | −11.491 | 2.891 | 1.00 | 46.58 | C |
| ATOM | 1214 | OD1 | ASP | A | 515 | −44.455 | −10.291 | 2.619 | 1.00 | 46.93 | O |
| ATOM | 1215 | OD2 | ASP | A | 515 | −45.421 | −12.211 | 2.191 | 1.00 | 46.93 | O |
| ATOM | 1216 | N | ILE | A | 516 | −41.853 | −10.442 | 5.643 | 1.00 | 46.29 | N |
| ATOM | 1217 | CA | ILE | A | 516 | −41.374 | −9.118 | 6.071 | 1.00 | 46.28 | C |
| ATOM | 1218 | C | ILE | A | 516 | −39.862 | −8.936 | 5.881 | 1.00 | 46.30 | C |
| ATOM | 1219 | O | ILE | A | 516 | −39.406 | −7.859 | 5.481 | 1.00 | 46.35 | O |
| ATOM | 1220 | CB | ILE | A | 516 | −41.801 | −8.796 | 7.533 | 1.00 | 46.32 | C |
| ATOM | 1221 | CG1 | ILE | A | 516 | −43.326 | −8.640 | 7.613 | 1.00 | 46.09 | C |
| ATOM | 1222 | CG2 | ILE | A | 516 | −41.078 | −7.538 | 8.060 | 1.00 | 46.34 | C |
| ATOM | 1223 | CD1 | ILE | A | 516 | −43.875 | −8.377 | 9.005 | 1.00 | 46.13 | C |
| ATOM | 1224 | N | THR | A | 517 | −39.102 | −9.994 | 6.166 | 1.00 | 46.31 | N |
| ATOM | 1225 | CA | THR | A | 517 | −37.647 | −10.012 | 5.983 | 1.00 | 46.30 | C |
| ATOM | 1226 | C | THR | A | 517 | −37.260 | −9.825 | 4.513 | 1.00 | 46.42 | C |
| ATOM | 1227 | O | THR | A | 517 | −36.402 | −8.999 | 4.189 | 1.00 | 46.53 | O |
| ATOM | 1228 | CB | THR | A | 517 | −37.037 | −11.330 | 6.524 | 1.00 | 46.30 | C |
| ATOM | 1229 | OG1 | THR | A | 517 | −37.206 | −11.389 | 7.946 | 1.00 | 46.02 | O |
| ATOM | 1230 | CG2 | THR | A | 517 | −35.551 | −11.442 | 6.187 | 1.00 | 46.52 | C |
| ATOM | 1231 | N | THR | A | 518 | −37.904 | −10.597 | 3.638 | 1.00 | 46.44 | N |
| ATOM | 1232 | CA | THR | A | 518 | −37.690 | −10.511 | 2.199 | 1.00 | 46.36 | C |
| ATOM | 1233 | C | THR | A | 518 | −37.985 | −9.097 | 1.703 | 1.00 | 46.46 | C |
| ATOM | 1234 | O | THR | A | 518 | −37.109 | −8.442 | 1.143 | 1.00 | 46.52 | O |
| ATOM | 1235 | CB | THR | A | 518 | −38.570 | −11.533 | 1.433 | 1.00 | 46.33 | C |
| ATOM | 1236 | OG1 | THR | A | 518 | −38.439 | −12.830 | 2.027 | 1.00 | 46.10 | O |
| ATOM | 1237 | CG2 | THR | A | 518 | −38.161 | −11.613 | −0.027 | 1.00 | 46.28 | C |
| ATOM | 1238 | N | LYS | A | 519 | −39.215 | −8.638 | 1.930 | 1.00 | 46.62 | N |
| ATOM | 1239 | CA | LYS | A | 519 | −39.675 | −7.318 | 1.486 | 1.00 | 46.75 | C |
| ATOM | 1240 | C | LYS | A | 519 | −38.768 | −6.165 | 1.928 | 1.00 | 46.79 | C |
| ATOM | 1241 | O | LYS | A | 519 | −38.148 | −5.502 | 1.095 | 1.00 | 46.72 | O |
| ATOM | 1242 | CB | LYS | A | 519 | −41.104 | −7.061 | 1.986 | 1.00 | 46.69 | C |
| ATOM | 1243 | CG | LYS | A | 519 | −42.196 | −7.033 | 0.922 | 1.00 | 46.77 | C |
| ATOM | 1244 | CD | LYS | A | 519 | −43.581 | −7.174 | 1.584 | 1.00 | 46.83 | C |
| ATOM | 1245 | CE | LYS | A | 519 | −44.644 | −6.269 | 0.962 | 1.00 | 46.37 | C |
| ATOM | 1246 | NZ | LYS | A | 519 | −44.959 | −6.623 | −0.445 | 1.00 | 46.22 | N |
| ATOM | 1247 | N | HIS | A | 520 | −38.679 | −5.950 | 3.239 | 1.00 | 46.83 | N |
| ATOM | 1248 | CA | HIS | A | 520 | −38.189 | −4.681 | 3.777 | 1.00 | 46.89 | C |
| ATOM | 1249 | C | HIS | A | 520 | −36.776 | −4.719 | 4.361 | 1.00 | 46.70 | C |
| ATOM | 1250 | O | HIS | A | 520 | −36.234 | −3.682 | 4.762 | 1.00 | 46.71 | O |
| ATOM | 1251 | CB | HIS | A | 520 | −39.191 | −4.130 | 4.801 | 1.00 | 46.98 | C |
| ATOM | 1252 | CG | HIS | A | 520 | −40.603 | −4.101 | 4.300 | 1.00 | 47.87 | C |
| ATOM | 1253 | ND1 | HIS | A | 520 | −41.022 | −3.245 | 3.302 | 1.00 | 48.63 | N |
| ATOM | 1254 | CD2 | HIS | A | 520 | −41.688 | −4.835 | 4.645 | 1.00 | 48.59 | C |
| ATOM | 1255 | CE1 | HIS | A | 520 | −42.304 | −3.448 | 3.058 | 1.00 | 48.64 | C |
| ATOM | 1256 | NE2 | HIS | A | 520 | −42.732 | −4.408 | 3.859 | 1.00 | 49.16 | N |
| ATOM | 1257 | N | MET | A | 521 | −36.173 | −5.903 | 4.400 | 1.00 | 46.39 | N |
| ATOM | 1258 | CA | MET | A | 521 | −34.835 | −6.029 | 4.966 | 1.00 | 46.03 | C |
| ATOM | 1259 | C | MET | A | 521 | −33.799 | −6.516 | 3.959 | 1.00 | 46.02 | C |
| ATOM | 1260 | O | MET | A | 521 | −32.745 | −5.898 | 3.817 | 1.00 | 46.24 | O |
| ATOM | 1261 | CB | MET | A | 521 | −34.847 | −6.898 | 6.226 | 1.00 | 45.99 | C |
| ATOM | 1262 | CG | MET | A | 521 | −35.861 | −6.438 | 7.264 | 1.00 | 45.73 | C |
| ATOM | 1263 | SD | MET | A | 521 | −35.460 | −6.883 | 8.959 | 1.00 | 45.73 | S |
| ATOM | 1264 | CE | MET | A | 521 | −35.560 | −8.669 | 8.915 | 1.00 | 45.51 | C |
| ATOM | 1265 | N | LEU | A | 522 | −34.104 | −7.600 | 3.249 | 1.00 | 45.85 | N |
| ATOM | 1266 | CA | LEU | A | 522 | −33.152 | −8.198 | 2.301 | 1.00 | 45.76 | C |
| ATOM | 1267 | C | LEU | A | 522 | −32.518 | −7.224 | 1.288 | 1.00 | 45.61 | C |
| ATOM | 1268 | O | LEU | A | 522 | −31.302 | −7.273 | 1.095 | 1.00 | 45.59 | O |
| ATOM | 1269 | CB | LEU | A | 522 | −33.758 | −9.412 | 1.578 | 1.00 | 45.80 | C |
| ATOM | 1270 | CG | LEU | A | 522 | −32.817 | −10.180 | 0.641 | 1.00 | 45.86 | C |
| ATOM | 1271 | CD1 | LEU | A | 522 | −31.852 | −11.083 | 1.409 | 1.00 | 45.84 | C |
| ATOM | 1272 | CD2 | LEU | A | 522 | −33.612 | −10.980 | −0.358 | 1.00 | 45.74 | C |
| ATOM | 1273 | N | PRO | A | 523 | −33.326 | −6.351 | 0.637 | 1.00 | 45.49 | N |
| ATOM | 1274 | CA | PRO | A | 523 | −32.736 | −5.431 | −0.347 | 1.00 | 45.39 | C |
| ATOM | 1275 | C | PRO | A | 523 | −31.592 | −4.603 | 0.229 | 1.00 | 45.28 | C |
| ATOM | 1276 | O | PRO | A | 523 | −30.577 | −4.403 | −0.444 | 1.00 | 45.34 | O |
| ATOM | 1277 | CB | PRO | A | 523 | −33.907 | −4.517 | −0.723 | 1.00 | 45.38 | C |
| ATOM | 1278 | CG | PRO | A | 523 | −35.109 | −5.330 | −0.469 | 1.00 | 45.53 | C |
| ATOM | 1279 | CD | PRO | A | 523 | −34.786 | −6.161 | 0.736 | 1.00 | 45.53 | C |
| ATOM | 1280 | N | THR | A | 524 | −31.758 | −4.143 | 1.468 | 1.00 | 45.05 | N |
| ATOM | 1281 | CA | THR | A | 524 | −30.743 | −3.344 | 2.152 | 1.00 | 44.69 | C |
| ATOM | 1282 | C | THR | A | 524 | −29.446 | −4.139 | 2.346 | 1.00 | 44.54 | C |
| ATOM | 1283 | O | THR | A | 524 | −28.356 | −3.613 | 2.109 | 1.00 | 44.58 | O |
| ATOM | 1284 | CB | THR | A | 524 | −31.285 | −2.783 | 3.489 | 1.00 | 44.63 | C |
| ATOM | 1285 | OG1 | THR | A | 524 | −32.363 | −1.877 | 3.215 | 1.00 | 44.47 | O |
| ATOM | 1286 | CG2 | THR | A | 524 | −30.207 | −2.040 | 4.256 | 1.00 | 44.46 | C |
| ATOM | 1287 | N | VAL | A | 525 | −29.574 | −5.404 | 2.746 | 1.00 | 44.17 | N |
| ATOM | 1288 | CA | VAL | A | 525 | −28.424 | −6.301 | 2.894 | 1.00 | 43.85 | C |
| ATOM | 1289 | C | VAL | A | 525 | −27.692 | −6.503 | 1.556 | 1.00 | 43.90 | C |
| ATOM | 1290 | O | VAL | A | 525 | −26.471 | −6.334 | 1.476 | 1.00 | 43.92 | O |
| ATOM | 1291 | CB | VAL | A | 525 | −28.837 | −7.676 | 3.477 | 1.00 | 43.68 | C |
| ATOM | 1292 | CG1 | VAL | A | 525 | −27.611 | −8.520 | 3.816 | 1.00 | 43.08 | C |
| ATOM | 1293 | CG2 | VAL | A | 525 | −29.706 | −7.493 | 4.700 | 1.00 | 43.54 | C |
| ATOM | 1294 | N | LEU | A | 526 | −28.444 | −6.848 | 0.513 | 1.00 | 43.76 | N |
| ATOM | 1295 | CA | LEU | A | 526 | −27.868 | −7.133 | −0.803 | 1.00 | 43.70 | C |
| ATOM | 1296 | C | LEU | A | 526 | −27.231 | −5.903 | −1.446 | 1.00 | 43.64 | C |
| ATOM | 1297 | O | LEU | A | 526 | −26.269 | −6.024 | −2.205 | 1.00 | 43.70 | O |
| ATOM | 1298 | CB | LEU | A | 526 | −28.914 | −7.747 | −1.745 | 1.00 | 43.60 | C |
| ATOM | 1299 | CG | LEU | A | 526 | −29.556 | −9.099 | −1.396 | 1.00 | 43.64 | C |
| ATOM | 1300 | CD1 | LEU | A | 526 | −30.588 | −9.467 | −2.454 | 1.00 | 43.84 | C |
| ATOM | 1301 | CD2 | LEU | A | 526 | −28.533 | −10.224 | −1.244 | 1.00 | 43.39 | C |
| ATOM | 1302 | N | ARG | A | 527 | −27.771 | −4.726 | −1.132 | 1.00 | 43.52 | N |
| ATOM | 1303 | CA | ARG | A | 527 | −27.219 | −3.455 | −1.605 | 1.00 | 43.44 | C |
| ATOM | 1304 | C | ARG | A | 527 | −25.904 | −3.100 | −0.887 | 1.00 | 43.07 | C |
| ATOM | 1305 | O | ARG | A | 527 | −25.098 | −2.324 | −1.402 | 1.00 | 43.13 | O |
| ATOM | 1306 | CB | ARG | A | 527 | −28.260 | −2.338 | −1.450 | 1.00 | 43.41 | C |
| ATOM | 1307 | CG | ARG | A | 527 | −27.865 | −0.980 | −2.029 | 1.00 | 43.78 | C |
| ATOM | 1308 | CD | ARG | A | 527 | −28.840 | 0.126 | −1.617 | 1.00 | 44.07 | C |
| ATOM | 1309 | NE | ARG | A | 527 | −29.235 | 0.035 | −0.206 | 1.00 | 45.68 | N |
| ATOM | 1310 | CZ | ARG | A | 527 | −28.538 | 0.516 | 0.824 | 1.00 | 45.73 | C |
| ATOM | 1311 | NH1 | ARG | A | 527 | −27.380 | 1.146 | 0.635 | 1.00 | 45.76 | N |
| ATOM | 1312 | NH2 | ARG | A | 527 | −29.008 | 0.364 | 2.057 | 1.00 | 45.28 | N |
| ATOM | 1313 | N | MET | A | 528 | −25.682 | −3.682 | 0.289 | 1.00 | 42.69 | N |
| ATOM | 1314 | CA | MET | A | 528 | −24.456 | −3.436 | 1.055 | 1.00 | 42.18 | C |
| ATOM | 1315 | C | MET | A | 528 | −23.285 | −4.319 | 0.626 | 1.00 | 42.04 | C |
| ATOM | 1316 | O | MET | A | 528 | −22.176 | −4.169 | 1.129 | 1.00 | 41.91 | O |
| ATOM | 1317 | CB | MET | A | 528 | −24.717 | −3.599 | 2.548 | 1.00 | 42.22 | C |
| ATOM | 1318 | CG | MET | A | 528 | −25.661 | −2.562 | 3.107 | 1.00 | 42.28 | C |
| ATOM | 1319 | SD | MET | A | 528 | −26.120 | −2.905 | 4.804 | 1.00 | 41.81 | S |
| ATOM | 1320 | CE | MET | A | 528 | −24.812 | −2.068 | 5.692 | 1.00 | 40.79 | C |
| ATOM | 1321 | N | ALA | A | 529 | −23.534 | −5.228 | −0.314 | 1.00 | 41.98 | N |
| ATOM | 1322 | CA | ALA | A | 529 | −22.477 | −6.065 | −0.888 | 1.00 | 41.82 | C |
| ATOM | 1323 | C | ALA | A | 529 | −21.374 | −5.239 | −1.575 | 1.00 | 41.65 | C |
| ATOM | 1324 | O | ALA | A | 529 | −20.241 | −5.703 | −1.714 | 1.00 | 41.33 | O |
| ATOM | 1325 | CB | ALA | A | 529 | −23.074 | −7.081 | −1.856 | 1.00 | 41.78 | C |
| ATOM | 1326 | N | GLY | A | 530 | −21.719 | −4.022 | −1.993 | 1.00 | 41.58 | N |
| ATOM | 1327 | CA | GLY | A | 530 | −20.760 | −3.095 | −2.581 | 1.00 | 41.79 | C |
| ATOM | 1328 | C | GLY | A | 530 | −20.374 | −1.966 | −1.641 | 1.00 | 42.07 | C |
| ATOM | 1329 | O | GLY | A | 530 | −20.378 | −0.800 | −2.029 | 1.00 | 42.13 | O |
| ATOM | 1330 | N | ASP | A | 531 | −20.052 | −2.311 | −0.396 | 1.00 | 42.27 | N |
| ATOM | 1331 | CA | ASP | A | 531 | −19.581 | −1.332 | 0.580 | 1.00 | 42.37 | C |
| ATOM | 1332 | C | ASP | A | 531 | −18.075 | −1.145 | 0.426 | 1.00 | 42.35 | C |
| ATOM | 1333 | O | ASP | A | 531 | −17.348 | −2.124 | 0.243 | 1.00 | 42.38 | O |
| ATOM | 1334 | CB | ASP | A | 531 | −19.914 | −1.793 | 2.001 | 1.00 | 42.53 | C |
| ATOM | 1335 | CG | ASP | A | 531 | −19.674 | −0.711 | 3.044 | 1.00 | 42.71 | C |
| ATOM | 1336 | OD1 | ASP | A | 531 | −18.506 | −0.500 | 3.449 | 1.00 | 41.64 | O |
| ATOM | 1337 | OD2 | ASP | A | 531 | −20.669 | −0.089 | 3.474 | 1.00 | 43.53 | O |
| ATOM | 1338 | N | PRO | A | 532 | −17.595 | 0.109 | 0.508 | 1.00 | 42.40 | N |
| ATOM | 1339 | CA | PRO | A | 532 | −16.155 | 0.340 | 0.323 | 1.00 | 42.60 | C |
| ATOM | 1340 | C | PRO | A | 532 | −15.282 | −0.315 | 1.399 | 1.00 | 42.96 | C |
| ATOM | 1341 | O | PRO | A | 532 | −14.090 | −0.519 | 1.177 | 1.00 | 43.07 | O |
| ATOM | 1342 | CB | PRO | A | 532 | −16.024 | 1.867 | 0.371 | 1.00 | 42.34 | C |
| ATOM | 1343 | CG | PRO | A | 532 | −17.252 | 2.344 | 1.058 | 1.00 | 42.63 | C |
| ATOM | 1344 | CD | PRO | A | 532 | −18.335 | 1.359 | 0.761 | 1.00 | 42.34 | C |
| ATOM | 1345 | N | VAL | A | 533 | −15.877 | −0.646 | 2.540 | 1.00 | 43.42 | N |
| ATOM | 1346 | CA | VAL | A | 533 | −15.141 | −1.210 | 3.659 | 1.00 | 43.94 | C |
| ATOM | 1347 | C | VAL | A | 533 | −15.372 | −2.717 | 3.753 | 1.00 | 44.44 | C |
| ATOM | 1348 | O | VAL | A | 533 | −16.514 | −3.185 | 3.804 | 1.00 | 44.63 | O |
| ATOM | 1349 | CB | VAL | A | 533 | −15.506 | −0.494 | 4.969 | 1.00 | 43.95 | C |
| ATOM | 1350 | CG1 | VAL | A | 533 | −14.967 | −1.246 | 6.182 | 1.00 | 44.36 | C |
| ATOM | 1351 | CG2 | VAL | A | 533 | −14.974 | 0.925 | 4.942 | 1.00 | 44.13 | C |
| ATOM | 1352 | N | ALA | A | 534 | −14.268 | −3.462 | 3.771 | 1.00 | 44.82 | N |
| ATOM | 1353 | CA | ALA | A | 534 | −14.284 | −4.922 | 3.745 | 1.00 | 44.97 | C |
| ATOM | 1354 | C | ALA | A | 534 | −14.994 | −5.539 | 4.948 | 1.00 | 45.37 | C |
| ATOM | 1355 | O | ALA | A | 534 | −15.621 | −6.602 | 4.817 | 1.00 | 45.51 | O |
| ATOM | 1356 | CB | ALA | A | 534 | −12.866 | −5.464 | 3.623 | 1.00 | 44.83 | C |
| ATOM | 1357 | N | ASN | A | 535 | −14.908 | −4.878 | 6.106 | 1.00 | 45.44 | N |
| ATOM | 1358 | CA | ASN | A | 535 | −15.586 | −5.363 | 7.316 | 1.00 | 45.51 | C |
| ATOM | 1359 | C | ASN | A | 535 | −17.099 | −5.402 | 7.163 | 1.00 | 45.59 | C |
| ATOM | 1360 | O | ASN | A | 535 | −17.764 | −6.279 | 7.718 | 1.00 | 45.65 | O |
| ATOM | 1361 | CB | ASN | A | 535 | −15.203 | −4.538 | 8.543 | 1.00 | 45.56 | C |
| ATOM | 1362 | CG | ASN | A | 535 | −13.704 | −4.454 | 8.741 | 1.00 | 45.50 | C |
| ATOM | 1363 | OD1 | ASN | A | 535 | −13.026 | −3.689 | 8.057 | 1.00 | 44.39 | O |
| ATOM | 1364 | ND2 | ASN | A | 535 | −13.179 | −5.238 | 9.684 | 1.00 | 44.89 | N |
| ATOM | 1365 | N | VAL | A | 536 | −17.636 | −4.453 | 6.401 | 1.00 | 45.63 | N |
| ATOM | 1366 | CA | VAL | A | 536 | −19.059 | −4.447 | 6.075 | 1.00 | 45.77 | C |
| ATOM | 1367 | C | VAL | A | 536 | −19.391 | −5.572 | 5.073 | 1.00 | 45.84 | C |
| ATOM | 1368 | O | VAL | A | 536 | −20.379 | −6.294 | 5.249 | 1.00 | 45.99 | O |
| ATOM | 1369 | CB | VAL | A | 536 | −19.525 | −3.055 | 5.546 | 1.00 | 45.82 | C |
| ATOM | 1370 | CG1 | VAL | A | 536 | −21.024 | −3.050 | 5.256 | 1.00 | 45.48 | C |
| ATOM | 1371 | CG2 | VAL | A | 536 | −19.171 | −1.945 | 6.540 | 1.00 | 45.27 | C |
| ATOM | 1372 | N | ARG | A | 537 | −18.547 | −5.731 | 4.051 | 1.00 | 45.88 | N |
| ATOM | 1373 | CA | ARG | A | 537 | −18.753 | −6.738 | 2.999 | 1.00 | 45.78 | C |
| ATOM | 1374 | C | ARG | A | 537 | −18.808 | −8.183 | 3.512 | 1.00 | 46.37 | C |
| ATOM | 1375 | O | ARG | A | 537 | −19.628 | −8.969 | 3.041 | 1.00 | 46.59 | O |
| ATOM | 1376 | CB | ARG | A | 537 | −17.706 | −6.612 | 1.881 | 1.00 | 45.82 | C |
| ATOM | 1377 | CG | ARG | A | 537 | −17.915 | −5.427 | 0.942 | 1.00 | 44.94 | C |
| ATOM | 1378 | CD | ARG | A | 537 | −17.147 | −5.568 | −0.386 | 1.00 | 44.59 | C |
| ATOM | 1379 | NE | ARG | A | 537 | −15.700 | −5.790 | −0.247 | 1.00 | 41.53 | N |
| ATOM | 1380 | CZ | ARG | A | 537 | −14.800 | −4.858 | 0.072 | 1.00 | 39.44 | C |
| ATOM | 1381 | NH1 | ARG | A | 537 | −15.165 | −3.606 | 0.320 | 1.00 | 38.21 | N |
| ATOM | 1382 | NH2 | ARG | A | 537 | −13.521 | −5.187 | 0.162 | 1.00 | 37.77 | N |
| ATOM | 1383 | N | PHE | A | 538 | −17.950 | −8.545 | 4.466 | 1.00 | 46.74 | N |
| ATOM | 1384 | CA | PHE | A | 538 | −18.005 | −9.908 | 4.994 | 1.00 | 46.90 | C |
| ATOM | 1385 | C | PHE | A | 538 | −19.083 | −10.104 | 6.052 | 1.00 | 47.18 | C |
| ATOM | 1386 | O | PHE | A | 538 | −19.476 | −11.239 | 6.332 | 1.00 | 47.57 | O |
| ATOM | 1387 | CB | PHE | A | 538 | −16.633 | −10.464 | 5.432 | 1.00 | 46.88 | C |
| ATOM | 1388 | CG | PHE | A | 538 | −16.058 | −9.835 | 6.674 | 1.00 | 46.97 | C |
| ATOM | 1389 | CD1 | PHE | A | 538 | −16.670 | −9.999 | 7.915 | 1.00 | 46.44 | C |
| ATOM | 1390 | CD2 | PHE | A | 538 | −14.858 | −9.122 | 6.607 | 1.00 | 47.38 | C |
| ATOM | 1391 | CE1 | PHE | A | 538 | −16.130 | −9.428 | 9.054 | 1.00 | 46.01 | C |
| ATOM | 1392 | CE2 | PHE | A | 538 | −14.304 | −8.554 | 7.745 | 1.00 | 46.57 | C |
| ATOM | 1393 | CZ | PHE | A | 538 | −14.943 | −8.706 | 8.969 | 1.00 | 46.69 | C |
| ATOM | 1394 | N | ASN | A | 539 | −19.570 | −9.010 | 6.634 | 1.00 | 47.17 | N |
| ATOM | 1395 | CA | ASN | A | 539 | −20.765 | −9.106 | 7.468 | 1.00 | 47.26 | C |
| ATOM | 1396 | C | ASN | A | 539 | −22.029 | −9.290 | 6.620 | 1.00 | 47.30 | C |
| ATOM | 1397 | O | ASN | A | 539 | −23.037 | −9.817 | 7.105 | 1.00 | 47.35 | O |
| ATOM | 1398 | CB | ASN | A | 539 | −20.890 | −7.925 | 8.436 | 1.00 | 47.30 | C |
| ATOM | 1399 | CG | ASN | A | 539 | −20.267 | −8.220 | 9.801 | 1.00 | 47.54 | C |
| ATOM | 1400 | OD1 | ASN | A | 539 | −20.607 | −9.206 | 10.453 | 1.00 | 47.99 | O |
| ATOM | 1401 | ND2 | ASN | A | 539 | −19.348 | −7.366 | 10.230 | 1.00 | 47.48 | N |
| ATOM | 1402 | N | VAL | A | 540 | −21.964 | −8.875 | 5.351 | 1.00 | 47.11 | N |
| ATOM | 1403 | CA | VAL | A | 540 | −23.043 | −9.150 | 4.396 | 1.00 | 46.93 | C |
| ATOM | 1404 | C | VAL | A | 540 | −23.146 | −10.656 | 4.149 | 1.00 | 46.86 | C |
| ATOM | 1405 | O | VAL | A | 540 | −24.227 | −11.233 | 4.269 | 1.00 | 46.70 | O |
| ATOM | 1406 | CB | VAL | A | 540 | −22.862 | −8.381 | 3.067 | 1.00 | 46.85 | C |
| ATOM | 1407 | CG1 | VAL | A | 540 | −23.814 | −8.897 | 2.003 | 1.00 | 46.51 | C |
| ATOM | 1408 | CG2 | VAL | A | 540 | −23.076 | −6.898 | 3.287 | 1.00 | 47.01 | C |
| ATOM | 1409 | N | ALA | A | 541 | −22.014 | −11.282 | 3.831 | 1.00 | 46.81 | N |
| ATOM | 1410 | CA | ALA | A | 541 | −21.945 | −12.731 | 3.645 | 1.00 | 46.96 | C |
| ATOM | 1411 | C | ALA | A | 541 | −22.323 | −13.507 | 4.915 | 1.00 | 47.06 | C |
| ATOM | 1412 | O | ALA | A | 541 | −22.926 | −14.575 | 4.837 | 1.00 | 47.23 | O |
| ATOM | 1413 | CB | ALA | A | 541 | −20.565 | −13.134 | 3.161 | 1.00 | 46.80 | C |
| ATOM | 1414 | N | LYS | A | 542 | −21.969 | −12.964 | 6.077 | 1.00 | 47.25 | N |
| ATOM | 1415 | CA | LYS | A | 542 | −22.348 | −13.557 | 7.361 | 1.00 | 47.33 | C |
| ATOM | 1416 | C | LYS | A | 542 | −23.847 | −13.421 | 7.636 | 1.00 | 47.27 | C |
| ATOM | 1417 | O | LYS | A | 542 | −24.450 | −14.313 | 8.236 | 1.00 | 47.36 | O |
| ATOM | 1418 | CB | LYS | A | 542 | −21.547 | −12.933 | 8.506 | 1.00 | 47.51 | C |
| ATOM | 1419 | CG | LYS | A | 542 | −20.137 | −13.478 | 8.661 | 1.00 | 47.72 | C |
| ATOM | 1420 | CD | LYS | A | 542 | −19.401 | −12.750 | 9.758 | 1.00 | 48.53 | C |
| ATOM | 1421 | CE | LYS | A | 542 | −18.417 | −13.679 | 10.432 | 1.00 | 50.17 | C |
| ATOM | 1422 | NZ | LYS | A | 542 | −17.891 | −13.141 | 11.725 | 1.00 | 51.67 | N |
| ATOM | 1423 | N | SER | A | 543 | −24.437 | −12.308 | 7.196 | 1.00 | 47.06 | N |
| ATOM | 1424 | CA | SER | A | 543 | −25.874 | −12.075 | 7.339 | 1.00 | 47.01 | C |
| ATOM | 1425 | C | SER | A | 543 | −26.714 | −13.065 | 6.529 | 1.00 | 47.14 | C |
| ATOM | 1426 | O | SER | A | 543 | −27.598 | −13.732 | 7.077 | 1.00 | 47.10 | O |
| ATOM | 1427 | CB | SER | A | 543 | −26.233 | −10.643 | 6.943 | 1.00 | 46.98 | C |
| ATOM | 1428 | OG | SER | A | 543 | −25.696 | −9.712 | 7.858 | 1.00 | 46.96 | O |
| ATOM | 1429 | N | LEU | A | 544 | −26.431 | −13.158 | 5.230 | 1.00 | 47.23 | N |
| ATOM | 1430 | CA | LEU | A | 544 | −27.161 | −14.050 | 4.329 | 1.00 | 47.37 | C |
| ATOM | 1431 | C | LEU | A | 544 | −27.114 | −15.499 | 4.805 | 1.00 | 47.62 | C |
| ATOM | 1432 | O | LEU | A | 544 | −28.107 | −16.218 | 4.707 | 1.00 | 47.72 | O |
| ATOM | 1433 | CB | LEU | A | 544 | −26.641 | −13.932 | 2.893 | 1.00 | 47.33 | C |
| ATOM | 1434 | CG | LEU | A | 544 | −26.576 | −12.534 | 2.261 | 1.00 | 47.08 | C |
| ATOM | 1435 | CD1 | LEU | A | 544 | −25.782 | −12.564 | 0.956 | 1.00 | 46.48 | C |
| ATOM | 1436 | CD2 | LEU | A | 544 | −27.965 | −11.939 | 2.047 | 1.00 | 46.81 | C |
| ATOM | 1437 | N | GLN | A | 545 | −25.965 | −15.919 | 5.331 | 1.00 | 47.88 | N |
| ATOM | 1438 | CA | GLN | A | 545 | −25.844 | −17.226 | 5.979 | 1.00 | 48.18 | C |
| ATOM | 1439 | C | GLN | A | 545 | −26.855 | −17.385 | 7.123 | 1.00 | 48.46 | C |
| ATOM | 1440 | O | GLN | A | 545 | −27.468 | −18.445 | 7.283 | 1.00 | 48.45 | O |
| ATOM | 1441 | CB | GLN | A | 545 | −24.414 | −17.447 | 6.492 | 1.00 | 48.09 | C |
| ATOM | 1442 | CG | GLN | A | 545 | −24.190 | −18.804 | 7.157 | 1.00 | 47.82 | C |
| ATOM | 1443 | CD | GLN | A | 545 | −22.731 | −19.097 | 7.449 | 1.00 | 48.02 | C |
| ATOM | 1444 | OE1 | GLN | A | 545 | −21.837 | −18.710 | 6.693 | 1.00 | 47.82 | O |
| ATOM | 1445 | NE2 | GLN | A | 545 | −22.483 | −19.798 | 8.549 | 1.00 | 47.75 | N |
| ATOM | 1446 | N | LYS | A | 546 | −27.028 | −16.320 | 7.903 | 1.00 | 48.88 | N |
| ATOM | 1447 | CA | LYS | A | 546 | −27.911 | −16.341 | 9.066 | 1.00 | 49.35 | C |
| ATOM | 1448 | C | LYS | A | 546 | −29.383 | −16.265 | 8.662 | 1.00 | 49.60 | C |
| ATOM | 1449 | O | LYS | A | 546 | −30.215 | −17.005 | 9.196 | 1.00 | 49.70 | O |
| ATOM | 1450 | CB | LYS | A | 546 | −27.561 | −15.193 | 10.020 | 1.00 | 49.22 | C |
| ATOM | 1451 | CG | LYS | A | 546 | −28.341 | −15.174 | 11.325 | 1.00 | 49.47 | C |
| ATOM | 1452 | CD | LYS | A | 546 | −27.840 | −16.241 | 12.297 | 1.00 | 50.23 | C |
| ATOM | 1453 | CE | LYS | A | 546 | −28.137 | −15.867 | 13.744 | 1.00 | 49.74 | C |
| ATOM | 1454 | NZ | LYS | A | 546 | −29.589 | −15.654 | 13.984 | 1.00 | 49.73 | N |
| ATOM | 1455 | N | ILE | A | 547 | −29.691 | −15.380 | 7.715 | 1.00 | 49.73 | N |
| ATOM | 1456 | CA | ILE | A | 547 | −31.077 | −15.100 | 7.337 | 1.00 | 49.85 | C |
| ATOM | 1457 | C | ILE | A | 547 | −31.617 | −16.028 | 6.232 | 1.00 | 50.26 | C |
| ATOM | 1458 | O | ILE | A | 547 | −32.828 | −16.237 | 6.128 | 1.00 | 50.28 | O |
| ATOM | 1459 | CB | ILE | A | 547 | −31.284 | −13.603 | 6.976 | 1.00 | 49.76 | C |
| ATOM | 1460 | CG1 | ILE | A | 547 | −30.649 | −13.263 | 5.624 | 1.00 | 49.54 | C |
| ATOM | 1461 | CG2 | ILE | A | 547 | −30.722 | −12.714 | 8.084 | 1.00 | 49.58 | C |
| ATOM | 1462 | CD1 | ILE | A | 547 | −31.108 | −11.946 | 5.041 | 1.00 | 49.57 | C |
| ATOM | 1463 | N | GLY | A | 548 | −30.707 | −16.587 | 5.433 | 1.00 | 50.64 | N |
| ATOM | 1464 | CA | GLY | A | 548 | −31.039 | −17.492 | 4.324 | 1.00 | 50.95 | C |
| ATOM | 1465 | C | GLY | A | 548 | −32.139 | −18.518 | 4.560 | 1.00 | 51.20 | C |
| ATOM | 1466 | O | GLY | A | 548 | −33.052 | −18.624 | 3.737 | 1.00 | 51.18 | O |
| ATOM | 1467 | N | PRO | A | 549 | −32.044 | −19.300 | 5.664 | 1.00 | 51.48 | N |
| ATOM | 1468 | CA | PRO | A | 549 | −33.087 | −20.263 | 6.076 | 1.00 | 51.69 | C |
| ATOM | 1469 | C | PRO | A | 549 | −34.502 | −19.690 | 6.194 | 1.00 | 51.84 | C |
| ATOM | 1470 | O | PRO | A | 549 | −35.473 | −20.436 | 6.084 | 1.00 | 51.82 | O |
| ATOM | 1471 | CB | PRO | A | 549 | −32.607 | −20.726 | 7.457 | 1.00 | 51.66 | C |
| ATOM | 1472 | CG | PRO | A | 549 | −31.138 | −20.599 | 7.394 | 1.00 | 51.56 | C |
| ATOM | 1473 | CD | PRO | A | 549 | −30.880 | −19.360 | 6.573 | 1.00 | 51.45 | C |
| ATOM | 1474 | N | ILE | A | 550 | −34.607 | −18.382 | 6.414 | 1.00 | 52.18 | N |
| ATOM | 1475 | CA | ILE | A | 550 | −35.896 | −17.711 | 6.601 | 1.00 | 52.49 | C |
| ATOM | 1476 | C | ILE | A | 550 | −36.395 | −17.133 | 5.264 | 1.00 | 52.62 | C |
| ATOM | 1477 | O | ILE | A | 550 | −37.220 | −16.217 | 5.223 | 1.00 | 52.64 | O |
| ATOM | 1478 | CB | ILE | A | 550 | −35.806 | −16.627 | 7.724 | 1.00 | 52.47 | C |
| ATOM | 1479 | CG1 | ILE | A | 550 | −34.925 | −17.131 | 8.874 | 1.00 | 52.70 | C |
| ATOM | 1480 | CG2 | ILE | A | 550 | −37.187 | −16.297 | 8.272 | 1.00 | 52.49 | C |
| ATOM | 1481 | CD1 | ILE | A | 550 | −34.067 | −16.066 | 9.548 | 1.00 | 53.11 | C |
| ATOM | 1482 | N | LEU | A | 551 | −35.890 | −17.698 | 4.170 | 1.00 | 52.86 | N |
| ATOM | 1483 | CA | LEU | A | 551 | −36.253 | −17.269 | 2.823 | 1.00 | 53.16 | C |
| ATOM | 1484 | C | LEU | A | 551 | −36.639 | −18.464 | 1.957 | 1.00 | 53.47 | C |
| ATOM | 1485 | O | LEU | A | 551 | −35.962 | −19.498 | 1.982 | 1.00 | 53.57 | O |
| ATOM | 1486 | CB | LEU | A | 551 | −35.085 | −16.530 | 2.167 | 1.00 | 53.00 | C |
| ATOM | 1487 | CG | LEU | A | 551 | −34.531 | −15.277 | 2.846 | 1.00 | 52.74 | C |
| ATOM | 1488 | CD1 | LEU | A | 551 | −33.118 | −14.981 | 2.360 | 1.00 | 52.37 | C |
| ATOM | 1489 | CD2 | LEU | A | 551 | −35.450 | −14.087 | 2.617 | 1.00 | 52.66 | C |
| ATOM | 1490 | N | ASP | A | 552 | −37.722 | −18.315 | 1.193 | 1.00 | 53.70 | N |
| ATOM | 1491 | CA | ASP | A | 552 | −38.141 | −19.331 | 0.220 | 1.00 | 53.96 | C |
| ATOM | 1492 | C | ASP | A | 552 | −37.013 | −19.585 | −0.766 | 1.00 | 53.97 | C |
| ATOM | 1493 | O | ASP | A | 552 | −36.154 | −18.726 | −0.965 | 1.00 | 54.11 | O |
| ATOM | 1494 | CB | ASP | A | 552 | −39.400 | −18.887 | −0.537 | 1.00 | 54.09 | C |
| ATOM | 1495 | CG | ASP | A | 552 | −39.166 | −17.649 | −1.396 | 1.00 | 54.43 | C |
| ATOM | 1496 | OD1 | ASP | A | 552 | −39.600 | −17.647 | −2.568 | 1.00 | 54.20 | O |
| ATOM | 1497 | OD2 | ASP | A | 552 | −38.543 | −16.683 | −0.896 | 1.00 | 54.82 | O |
| ATOM | 1498 | N | ASN | A | 553 | −37.022 | −20.757 | −1.391 | 1.00 | 53.96 | N |
| ATOM | 1499 | CA | ASN | A | 553 | −35.917 | −21.147 | −2.254 | 1.00 | 54.02 | C |
| ATOM | 1500 | C | ASN | A | 553 | −35.789 | −20.294 | −3.525 | 1.00 | 54.02 | C |
| ATOM | 1501 | O | ASN | A | 553 | −34.710 | −20.225 | −4.116 | 1.00 | 54.17 | O |
| ATOM | 1502 | CB | ASN | A | 553 | −35.988 | −22.640 | −2.582 | 1.00 | 54.04 | C |
| ATOM | 1503 | CG | ASN | A | 553 | −34.621 | −23.298 | −2.594 | 1.00 | 54.26 | C |
| ATOM | 1504 | OD1 | ASN | A | 553 | −33.789 | −23.029 | −3.464 | 1.00 | 54.33 | O |
| ATOM | 1505 | ND2 | ASN | A | 553 | −34.383 | −24.170 | −1.622 | 1.00 | 54.49 | N |
| ATOM | 1506 | N | SER | A | 554 | −36.879 | −19.637 | −3.927 | 1.00 | 53.88 | N |
| ATOM | 1507 | CA | SER | A | 554 | −36.878 | −18.753 | −5.103 | 1.00 | 53.65 | C |
| ATOM | 1508 | C | SER | A | 554 | −36.045 | −17.490 | −4.872 | 1.00 | 53.44 | C |
| ATOM | 1509 | O | SER | A | 554 | −35.396 | −16.981 | −5.786 | 1.00 | 53.43 | O |
| ATOM | 1510 | CB | SER | A | 554 | −38.310 | −18.372 | −5.495 | 1.00 | 53.70 | C |
| ATOM | 1511 | OG | SER | A | 554 | −38.340 | −17.590 | −6.678 | 1.00 | 53.59 | O |
| ATOM | 1512 | N | THR | A | 555 | −36.072 | −16.995 | −3.640 | 1.00 | 53.17 | N |
| ATOM | 1513 | CA | THR | A | 555 | −35.329 | −15.801 | −3.261 | 1.00 | 52.97 | C |
| ATOM | 1514 | C | THR | A | 555 | −33.829 | −16.084 | −3.126 | 1.00 | 52.77 | C |
| ATOM | 1515 | O | THR | A | 555 | −32.997 | −15.245 | −3.483 | 1.00 | 52.75 | O |
| ATOM | 1516 | CB | THR | A | 555 | −35.896 | −15.211 | −1.953 | 1.00 | 53.05 | C |
| ATOM | 1517 | OG1 | THR | A | 555 | −37.268 | −14.843 | −2.157 | 1.00 | 53.12 | O |
| ATOM | 1518 | CG2 | THR | A | 555 | −35.113 | −13.992 | −1.508 | 1.00 | 52.96 | C |
| ATOM | 1519 | N | LEU | A | 556 | −33.496 | −17.273 | −2.627 | 1.00 | 52.47 | N |
| ATOM | 1520 | CA | LEU | A | 556 | −32.104 | −17.672 | −2.419 | 1.00 | 52.22 | C |
| ATOM | 1521 | C | LEU | A | 556 | −31.304 | −17.867 | −3.710 | 1.00 | 52.03 | C |
| ATOM | 1522 | O | LEU | A | 556 | −30.078 | −17.733 | −3.710 | 1.00 | 52.02 | O |
| ATOM | 1523 | CB | LEU | A | 556 | −32.033 | −18.950 | −1.574 | 1.00 | 52.26 | C |
| ATOM | 1524 | CG | LEU | A | 556 | −32.063 | −18.815 | −0.048 | 1.00 | 52.29 | C |
| ATOM | 1525 | CD1 | LEU | A | 556 | −32.291 | −20.169 | 0.608 | 1.00 | 52.41 | C |
| ATOM | 1526 | CD2 | LEU | A | 556 | −30.778 | −18.180 | 0.472 | 1.00 | 52.63 | C |
| ATOM | 1527 | N | GLN | A | 557 | −32.001 | −18.168 | −4.804 | 1.00 | 51.74 | N |
| ATOM | 1528 | CA | GLN | A | 557 | −31.353 | −18.608 | −6.041 | 1.00 | 51.44 | C |
| ATOM | 1529 | C | GLN | A | 557 | −31.138 | −17.503 | −7.076 | 1.00 | 51.13 | C |
| ATOM | 1530 | O | GLN | A | 557 | −30.094 | −17.459 | −7.729 | 1.00 | 51.08 | O |
| ATOM | 1531 | CB | GLN | A | 557 | −32.143 | −19.760 | −6.667 | 1.00 | 51.53 | C |
| ATOM | 1532 | CG | GLN | A | 557 | −32.303 | −20.983 | −5.767 | 1.00 | 51.72 | C |
| ATOM | 1533 | CD | GLN | A | 557 | −31.085 | −21.891 | −5.749 | 1.00 | 52.13 | C |
| ATOM | 1534 | OE1 | GLN | A | 557 | −30.228 | −21.832 | −6.635 | 1.00 | 52.14 | O |
| ATOM | 1535 | NE2 | GLN | A | 557 | −31.011 | −22.749 | −4.738 | 1.00 | 52.28 | N |
| ATOM | 1536 | N | SER | A | 558 | −32.125 | −16.625 | −7.227 | 1.00 | 50.72 | N |
| ATOM | 1537 | CA | SER | A | 558 | −32.081 | −15.579 | −8.247 | 1.00 | 50.32 | C |
| ATOM | 1538 | C | SER | A | 558 | −31.705 | −14.220 | −7.670 | 1.00 | 49.98 | C |
| ATOM | 1539 | O | SER | A | 558 | −31.227 | −13.344 | −8.391 | 1.00 | 49.97 | O |
| ATOM | 1540 | CB | SER | A | 558 | −33.432 | −15.471 | −8.951 | 1.00 | 50.38 | C |
| ATOM | 1541 | OG | SER | A | 558 | −34.422 | −14.987 | −8.059 | 1.00 | 50.37 | O |
| ATOM | 1542 | N | GLU | A | 559 | −31.930 | −14.055 | −6.369 | 1.00 | 49.57 | N |
| ATOM | 1543 | CA | GLU | A | 559 | −31.712 | −12.778 | −5.694 | 1.00 | 49.13 | C |
| ATOM | 1544 | C | GLU | A | 559 | −30.461 | −12.806 | −4.813 | 1.00 | 48.90 | C |
| ATOM | 1545 | O | GLU | A | 559 | −29.654 | −11.870 | −4.836 | 1.00 | 48.93 | O |
| ATOM | 1546 | CB | GLU | A | 559 | −32.943 | −12.398 | −4.864 | 1.00 | 49.09 | C |
| ATOM | 1547 | CG | GLU | A | 559 | −34.255 | −12.401 | −5.652 | 1.00 | 49.19 | C |
| ATOM | 1548 | CD | GLU | A | 559 | −35.489 | −12.430 | −4.765 | 1.00 | 49.05 | C |
| ATOM | 1549 | OE1 | GLU | A | 559 | −35.558 | −11.636 | −3.800 | 1.00 | 49.30 | O |
| ATOM | 1550 | OE2 | GLU | A | 559 | −36.397 | −13.242 | −5.041 | 1.00 | 48.26 | O |
| ATOM | 1551 | N | VAL | A | 560 | −30.303 | −13.885 | −4.048 | 1.00 | 48.48 | N |
| ATOM | 1552 | CA | VAL | A | 560 | −29.192 | −14.006 | −3.100 | 1.00 | 48.03 | C |
| ATOM | 1553 | C | VAL | A | 560 | −27.933 | −14.601 | −3.741 | 1.00 | 47.64 | C |
| ATOM | 1554 | O | VAL | A | 560 | −26.897 | −13.941 | −3.793 | 1.00 | 47.56 | O |
| ATOM | 1555 | CB | VAL | A | 560 | −29.600 | −14.802 | −1.826 | 1.00 | 47.99 | C |
| ATOM | 1556 | CG1 | VAL | A | 560 | −28.421 | −14.950 | −0.880 | 1.00 | 47.88 | C |
| ATOM | 1557 | CG2 | VAL | A | 560 | −30.762 | −14.116 | −1.115 | 1.00 | 47.91 | C |
| ATOM | 1558 | N | LYS | A | 561 | −28.034 | −15.832 | −4.241 | 1.00 | 47.25 | N |
| ATOM | 1559 | CA | LYS | A | 561 | −26.867 | −16.569 | −4.745 | 1.00 | 46.89 | C |
| ATOM | 1560 | C | LYS | A | 561 | −25.925 | −15.764 | −5.662 | 1.00 | 46.58 | C |
| ATOM | 1561 | O | LYS | A | 561 | −24.716 | −15.768 | −5.424 | 1.00 | 46.58 | O |
| ATOM | 1562 | CB | LYS | A | 561 | −27.267 | −17.899 | −5.399 | 1.00 | 46.86 | C |
| ATOM | 1563 | CG | LYS | A | 561 | −26.165 | −18.946 | −5.342 | 1.00 | 46.77 | C |
| ATOM | 1564 | CD | LYS | A | 561 | −26.286 | −19.958 | −6.457 | 1.00 | 46.94 | C |
| ATOM | 1565 | CE | LYS | A | 561 | −25.040 | −20.822 | −6.527 | 1.00 | 47.10 | C |
| ATOM | 1566 | NZ | LYS | A | 561 | −24.937 | −21.529 | −7.833 | 1.00 | 47.31 | N |
| ATOM | 1567 | N | PRO | A | 562 | −26.464 | −15.070 | −6.694 | 1.00 | 46.23 | N |
| ATOM | 1568 | CA | PRO | A | 562 | −25.586 | −14.266 | −7.554 | 1.00 | 46.03 | C |
| ATOM | 1569 | C | PRO | A | 562 | −24.696 | −13.290 | −6.778 | 1.00 | 45.83 | C |
| ATOM | 1570 | O | PRO | A | 562 | −23.530 | −13.105 | −7.134 | 1.00 | 45.76 | O |
| ATOM | 1571 | CB | PRO | A | 562 | −26.572 | −13.493 | −8.437 | 1.00 | 45.99 | C |
| ATOM | 1572 | CG | PRO | A | 562 | −27.767 | −14.347 | −8.487 | 1.00 | 46.06 | C |
| ATOM | 1573 | CD | PRO | A | 562 | −27.869 | −14.983 | −7.138 | 1.00 | 46.16 | C |
| ATOM | 1574 | N | ILE | A | 563 | −25.244 | −12.685 | −5.728 | 1.00 | 45.58 | N |
| ATOM | 1575 | CA | ILE | A | 563 | −24.500 | −11.739 | −4.899 | 1.00 | 45.50 | C |
| ATOM | 1576 | C | ILE | A | 563 | −23.349 | −12.420 | −4.138 | 1.00 | 45.61 | C |
| ATOM | 1577 | O | ILE | A | 563 | −22.222 | −11.910 | −4.125 | 1.00 | 45.71 | O |
| ATOM | 1578 | CB | ILE | A | 563 | −25.452 | −10.953 | −3.942 | 1.00 | 45.46 | C |
| ATOM | 1579 | CG1 | ILE | A | 563 | −26.397 | −10.035 | −4.741 | 1.00 | 45.12 | C |
| ATOM | 1580 | CG2 | ILE | A | 563 | −24.673 | −10.198 | −2.858 | 1.00 | 44.82 | C |
| ATOM | 1581 | CD1 | ILE | A | 563 | −25.715 | −9.029 | −5.670 | 1.00 | 44.38 | C |
| ATOM | 1582 | N | LEU | A | 564 | −23.632 | −13.573 | −3.528 | 1.00 | 45.57 | N |
| ATOM | 1583 | CA | LEU | A | 564 | −22.605 | −14.372 | −2.846 | 1.00 | 45.40 | C |
| ATOM | 1584 | C | LEU | A | 564 | −21.516 | −14.860 | −3.806 | 1.00 | 45.26 | C |
| ATOM | 1585 | O | LEU | A | 564 | −20.346 | −14.958 | −3.429 | 1.00 | 45.09 | O |
| ATOM | 1586 | CB | LEU | A | 564 | −23.232 | −15.563 | −2.108 | 1.00 | 45.48 | C |
| ATOM | 1587 | CG | LEU | A | 564 | −23.770 | −15.373 | −0.684 | 1.00 | 45.14 | C |
| ATOM | 1588 | CD1 | LEU | A | 564 | −24.546 | −16.600 | −0.240 | 1.00 | 44.98 | C |
| ATOM | 1589 | CD2 | LEU | A | 564 | −22.651 | −15.091 | 0.295 | 1.00 | 45.06 | C |
| ATOM | 1590 | N | GLU | A | 565 | −21.915 | −15.164 | −5.040 | 1.00 | 45.21 | N |
| ATOM | 1591 | CA | GLU | A | 565 | −20.976 | −15.534 | −6.101 | 1.00 | 45.14 | C |
| ATOM | 1592 | C | GLU | A | 565 | −20.006 | −14.392 | −6.400 | 1.00 | 45.00 | C |
| ATOM | 1593 | O | GLU | A | 565 | −18.807 | −14.624 | −6.559 | 1.00 | 45.00 | O |
| ATOM | 1594 | CB | GLU | A | 565 | −21.718 | −15.965 | −7.376 | 1.00 | 45.10 | C |
| ATOM | 1595 | CG | GLU | A | 565 | −22.242 | −17.402 | −7.349 | 1.00 | 45.16 | C |
| ATOM | 1596 | CD | GLU | A | 565 | −23.195 | −17.725 | −8.499 | 1.00 | 45.33 | C |
| ATOM | 1597 | OE1 | GLU | A | 565 | −23.175 | −18.876 | −8.988 | 1.00 | 45.37 | O |
| ATOM | 1598 | OE2 | GLU | A | 565 | −23.971 | −16.837 | −8.914 | 1.00 | 45.67 | O |
| ATOM | 1599 | N | LYS | A | 566 | −20.523 | −13.165 | −6.460 | 1.00 | 44.86 | N |
| ATOM | 1600 | CA | LYS | A | 566 | −19.679 | −11.984 | −6.661 | 1.00 | 44.85 | C |
| ATOM | 1601 | C | LYS | A | 566 | −18.679 | −11.802 | −5.517 | 1.00 | 44.47 | C |
| ATOM | 1602 | O | LYS | A | 566 | −17.496 | −11.551 | −5.761 | 1.00 | 44.52 | O |
| ATOM | 1603 | CB | LYS | A | 566 | −20.526 | −10.719 | −6.836 | 1.00 | 44.90 | C |
| ATOM | 1604 | CG | LYS | A | 566 | −19.801 | −9.589 | −7.574 | 1.00 | 45.43 | C |
| ATOM | 1605 | CD | LYS | A | 566 | −20.659 | −8.328 | −7.725 | 1.00 | 45.49 | C |
| ATOM | 1606 | CE | LYS | A | 566 | −20.605 | −7.440 | −6.481 | 1.00 | 46.48 | C |
| ATOM | 1607 | NZ | LYS | A | 566 | −21.139 | −6.079 | −6.772 | 1.00 | 46.96 | N |
| ATOM | 1608 | N | LEU | A | 567 | −19.157 | −11.954 | −4.281 | 1.00 | 43.95 | N |
| ATOM | 1609 | CA | LEU | A | 567 | −18.328 | −11.765 | −3.093 | 1.00 | 43.57 | C |
| ATOM | 1610 | C | LEU | A | 567 | −17.126 | −12.706 | −3.002 | 1.00 | 43.48 | C |
| ATOM | 1611 | O | LEU | A | 567 | −16.071 | −12.307 | −2.525 | 1.00 | 43.47 | O |
| ATOM | 1612 | CB | LEU | A | 567 | −19.169 | −11.860 | −1.811 | 1.00 | 43.69 | C |
| ATOM | 1613 | CG | LEU | A | 567 | −20.160 | −10.750 | −1.431 | 1.00 | 43.12 | C |
| ATOM | 1614 | CD1 | LEU | A | 567 | −20.824 | −11.097 | −0.111 | 1.00 | 42.00 | C |
| ATOM | 1615 | CD2 | LEU | A | 567 | −19.504 | −9.367 | −1.363 | 1.00 | 41.87 | C |
| ATOM | 1616 | N | THR | A | 568 | −17.276 | −13.949 | −3.450 | 1.00 | 43.46 | N |
| ATOM | 1617 | CA | THR | A | 568 | −16.142 | −14.886 | −3.480 | 1.00 | 43.55 | C |
| ATOM | 1618 | C | THR | A | 568 | −14.995 | −14.370 | −4.361 | 1.00 | 43.56 | C |
| ATOM | 1619 | O | THR | A | 568 | −13.837 | −14.756 | −4.183 | 1.00 | 43.52 | O |
| ATOM | 1620 | CB | THR | A | 568 | −16.550 | −16.291 | −3.973 | 1.00 | 43.54 | C |
| ATOM | 1621 | OG1 | THR | A | 568 | −17.025 | −16.208 | −5.321 | 1.00 | 43.54 | O |
| ATOM | 1622 | CG2 | THR | A | 568 | −17.636 | −16.888 | −3.088 | 1.00 | 43.64 | C |
| ATOM | 1623 | N | GLN | A | 569 | −15.327 | −13.494 | −5.304 | 1.00 | 43.54 | N |
| ATOM | 1624 | CA | GLN | A | 569 | −14.323 | −12.875 | −6.164 | 1.00 | 43.63 | C |
| ATOM | 1625 | C | GLN | A | 569 | −13.840 | −11.516 | −5.627 | 1.00 | 43.59 | C |
| ATOM | 1626 | O | GLN | A | 569 | −13.223 | −10.741 | −6.359 | 1.00 | 43.72 | O |
| ATOM | 1627 | CB | GLN | A | 569 | −14.846 | −12.737 | −7.601 | 1.00 | 43.63 | C |
| ATOM | 1628 | CG | GLN | A | 569 | −15.426 | −14.016 | −8.223 | 1.00 | 43.88 | C |
| ATOM | 1629 | CD | GLN | A | 569 | −14.481 | −15.202 | −8.152 | 1.00 | 44.17 | C |
| ATOM | 1630 | OE1 | GLN | A | 569 | −13.338 | −15.133 | −8.607 | 1.00 | 44.44 | O |
| ATOM | 1631 | NE2 | GLN | A | 569 | −14.959 | −16.304 | −7.580 | 1.00 | 44.13 | N |
| ATOM | 1632 | N | ASP | A | 570 | −14.117 | −11.242 | −4.352 | 1.00 | 43.48 | N |
| ATOM | 1633 | CA | ASP | A | 570 | −13.669 | −10.012 | −3.678 | 1.00 | 43.55 | C |
| ATOM | 1634 | C | ASP | A | 570 | −12.141 | −9.916 | −3.593 | 1.00 | 43.38 | C |
| ATOM | 1635 | O | ASP | A | 570 | −11.447 | −10.928 | −3.639 | 1.00 | 43.25 | O |
| ATOM | 1636 | CB | ASP | A | 570 | −14.264 | −9.938 | −2.264 | 1.00 | 43.56 | C |
| ATOM | 1637 | CG | ASP | A | 570 | −14.215 | −8.541 | −1.674 | 1.00 | 43.71 | C |
| ATOM | 1638 | OD1 | ASP | A | 570 | −13.397 | −8.310 | −0.764 | 1.00 | 44.52 | O |
| ATOM | 1639 | OD2 | ASP | A | 570 | −14.987 | −7.668 | −2.116 | 1.00 | 43.99 | O |
| ATOM | 1640 | N | GLN | A | 571 | −11.629 | −8.696 | −3.461 | 1.00 | 43.27 | N |
| ATOM | 1641 | CA | GLN | A | 571 | −10.193 | −8.471 | −3.326 | 1.00 | 43.47 | C |
| ATOM | 1642 | C | GLN | A | 571 | −9.636 | −8.892 | −1.965 | 1.00 | 43.76 | C |
| ATOM | 1643 | O | GLN | A | 571 | −8.457 | −9.227 | −1.856 | 1.00 | 43.65 | O |
| ATOM | 1644 | CB | GLN | A | 571 | −9.872 | −7.006 | −3.584 | 1.00 | 43.40 | C |
| ATOM | 1645 | CG | GLN | A | 571 | −10.088 | −6.591 | −5.029 | 1.00 | 43.83 | C |
| ATOM | 1646 | CD | GLN | A | 571 | −10.165 | −5.089 | −5.207 | 1.00 | 43.92 | C |
| ATOM | 1647 | OE1 | GLN | A | 571 | −9.739 | −4.323 | −4.336 | 1.00 | 44.69 | O |
| ATOM | 1648 | NE2 | GLN | A | 571 | −10.718 | −4.656 | −6.339 | 1.00 | 42.68 | N |
| ATOM | 1649 | N | ASP | A | 572 | −10.504 | −8.882 | −0.949 | 1.00 | 44.14 | N |
| ATOM | 1650 | CA | ASP | A | 572 | −10.153 | −9.165 | 0.449 | 1.00 | 44.38 | C |
| ATOM | 1651 | C | ASP | A | 572 | −10.210 | −10.645 | 0.830 | 1.00 | 44.68 | C |
| ATOM | 1652 | O | ASP | A | 572 | −11.141 | −11.357 | 0.447 | 1.00 | 44.77 | O |
| ATOM | 1653 | CB | ASP | A | 572 | −11.094 | −8.395 | 1.374 | 1.00 | 44.37 | C |
| ATOM | 1654 | CG | ASP | A | 572 | −10.732 | −8.550 | 2.828 | 1.00 | 44.30 | C |
| ATOM | 1655 | OD1 | ASP | A | 572 | −11.554 | −9.087 | 3.593 | 1.00 | 44.68 | O |
| ATOM | 1656 | OD2 | ASP | A | 572 | −9.614 | −8.146 | 3.207 | 1.00 | 45.46 | O |
| ATOM | 1657 | N | VAL | A | 573 | −9.225 | −11.080 | 1.618 | 1.00 | 45.04 | N |
| ATOM | 1658 | CA | VAL | A | 573 | −9.125 | −12.461 | 2.120 | 1.00 | 45.43 | C |
| ATOM | 1659 | C | VAL | A | 573 | −10.386 | −12.901 | 2.863 | 1.00 | 45.45 | C |
| ATOM | 1660 | O | VAL | A | 573 | −10.930 | −13.983 | 2.607 | 1.00 | 45.61 | O |
| ATOM | 1661 | CB | VAL | A | 573 | −7.898 | −12.650 | 3.073 | 1.00 | 45.40 | C |
| ATOM | 1662 | CG1 | VAL | A | 573 | −7.779 | −14.099 | 3.523 | 1.00 | 45.79 | C |
| ATOM | 1663 | CG2 | VAL | A | 573 | −6.599 | −12.212 | 2.396 | 1.00 | 46.45 | C |
| ATOM | 1664 | N | ASP | A | 574 | −10.839 | −12.054 | 3.782 | 1.00 | 45.40 | N |
| ATOM | 1665 | CA | ASP | A | 574 | −11.956 | −12.379 | 4.654 | 1.00 | 45.37 | C |
| ATOM | 1666 | C | ASP | A | 574 | −13.289 | −12.444 | 3.917 | 1.00 | 45.24 | C |
| ATOM | 1667 | O | ASP | A | 574 | −14.019 | −13.429 | 4.048 | 1.00 | 45.40 | O |
| ATOM | 1668 | CB | ASP | A | 574 | −12.018 | −11.394 | 5.823 | 1.00 | 45.56 | C |
| ATOM | 1669 | CG | ASP | A | 574 | −10.862 | −11.567 | 6.784 | 1.00 | 45.80 | C |
| ATOM | 1670 | OD1 | ASP | A | 574 | −10.569 | −12.715 | 7.172 | 1.00 | 46.51 | O |
| ATOM | 1671 | OD2 | ASP | A | 574 | −10.238 | −10.558 | 7.153 | 1.00 | 46.86 | O |
| ATOM | 1672 | N | VAL | A | 575 | −13.596 | −11.411 | 3.137 | 1.00 | 44.99 | N |
| ATOM | 1673 | CA | VAL | A | 575 | −14.835 | −11.379 | 2.357 | 1.00 | 44.82 | C |
| ATOM | 1674 | C | VAL | A | 575 | −15.010 | −12.649 | 1.508 | 1.00 | 44.68 | C |
| ATOM | 1675 | O | VAL | A | 575 | −16.096 | −13.235 | 1.502 | 1.00 | 44.53 | O |
| ATOM | 1676 | CB | VAL | A | 575 | −14.946 | −10.108 | 1.487 | 1.00 | 44.82 | C |
| ATOM | 1677 | CG1 | VAL | A | 575 | −16.299 | −10.058 | 0.773 | 1.00 | 44.95 | C |
| ATOM | 1678 | CG2 | VAL | A | 575 | −14.757 | −8.866 | 2.338 | 1.00 | 44.47 | C |
| ATOM | 1679 | N | LYS | A | 576 | −13.945 | −13.069 | 0.818 | 1.00 | 44.52 | N |
| ATOM | 1680 | CA | LYS | A | 576 | −13.934 | −14.347 | 0.090 | 1.00 | 44.55 | C |
| ATOM | 1681 | C | LYS | A | 576 | −14.373 | −15.477 | 1.012 | 1.00 | 44.11 | C |
| ATOM | 1682 | O | LYS | A | 576 | −15.397 | −16.122 | 0.765 | 1.00 | 44.01 | O |
| ATOM | 1683 | CB | LYS | A | 576 | −12.541 | −14.697 | −0.451 | 1.00 | 44.44 | C |
| ATOM | 1684 | CG | LYS | A | 576 | −11.976 | −13.765 | −1.504 | 1.00 | 45.43 | C |
| ATOM | 1685 | CD | LYS | A | 576 | −10.536 | −14.152 | −1.852 | 1.00 | 45.31 | C |
| ATOM | 1686 | CE | LYS | A | 576 | −9.756 | −12.978 | −2.437 | 1.00 | 46.02 | C |
| ATOM | 1687 | NZ | LYS | A | 576 | −8.323 | −13.308 | −2.693 | 1.00 | 46.53 | N |
| ATOM | 1688 | N | TYR | A | 577 | −13.601 | −15.696 | 2.080 | 1.00 | 43.62 | N |
| ATOM | 1689 | CA | TYR | A | 577 | −13.800 | −16.852 | 2.946 | 1.00 | 43.21 | C |
| ATOM | 1690 | C | TYR | A | 577 | −15.249 | −16.991 | 3.406 | 1.00 | 42.88 | C |
| ATOM | 1691 | O | TYR | A | 577 | −15.839 | −18.067 | 3.283 | 1.00 | 42.69 | O |
| ATOM | 1692 | CB | TYR | A | 577 | −12.847 | −16.848 | 4.152 | 1.00 | 43.24 | C |
| ATOM | 1693 | CG | TYR | A | 577 | −13.179 | −17.946 | 5.144 | 1.00 | 43.46 | C |
| ATOM | 1694 | CD1 | TYR | A | 577 | −12.601 | −19.213 | 5.039 | 1.00 | 43.39 | C |
| ATOM | 1695 | CD2 | TYR | A | 577 | −14.101 | −17.726 | 6.169 | 1.00 | 43.34 | C |
| ATOM | 1696 | CE1 | TYR | A | 577 | −12.923 | −20.230 | 5.945 | 1.00 | 43.19 | C |
| ATOM | 1697 | CE2 | TYR | A | 577 | −14.433 | −18.731 | 7.067 | 1.00 | 43.77 | C |
| ATOM | 1698 | CZ | TYR | A | 577 | −13.844 | −19.982 | 6.955 | 1.00 | 43.66 | C |
| ATOM | 1699 | OH | TYR | A | 577 | −14.187 | −20.969 | 7.864 | 1.00 | 43.71 | O |
| ATOM | 1700 | N | PHE | A | 578 | −15.811 | −15.903 | 3.926 | 1.00 | 42.51 | N |
| ATOM | 1701 | CA | PHE | A | 578 | −17.159 | −15.923 | 4.484 | 1.00 | 42.37 | C |
| ATOM | 1702 | C | PHE | A | 578 | −18.275 | −15.991 | 3.432 | 1.00 | 42.28 | C |
| ATOM | 1703 | O | PHE | A | 578 | −19.401 | −16.401 | 3.744 | 1.00 | 42.06 | O |
| ATOM | 1704 | CB | PHE | A | 578 | −17.357 | −14.737 | 5.423 | 1.00 | 42.30 | C |
| ATOM | 1705 | CG | PHE | A | 578 | −16.505 | −14.800 | 6.658 | 1.00 | 42.80 | C |
| ATOM | 1706 | CD1 | PHE | A | 578 | −16.878 | −15.604 | 7.739 | 1.00 | 42.53 | C |
| ATOM | 1707 | CD2 | PHE | A | 578 | −15.330 | −14.056 | 6.749 | 1.00 | 42.68 | C |
| ATOM | 1708 | CE1 | PHE | A | 578 | −16.092 | −15.669 | 8.887 | 1.00 | 41.63 | C |
| ATOM | 1709 | CE2 | PHE | A | 578 | −14.541 | −14.109 | 7.898 | 1.00 | 42.75 | C |
| ATOM | 1710 | CZ | PHE | A | 578 | −14.924 | −14.920 | 8.970 | 1.00 | 42.11 | C |
| ATOM | 1711 | N | ALA | A | 579 | −17.958 | −15.592 | 2.197 | 1.00 | 42.15 | N |
| ATOM | 1712 | CA | ALA | A | 579 | −18.882 | −15.737 | 1.064 | 1.00 | 41.78 | C |
| ATOM | 1713 | C | ALA | A | 579 | −18.977 | −17.201 | 0.657 | 1.00 | 41.60 | C |
| ATOM | 1714 | O | ALA | A | 579 | −20.063 | −17.701 | 0.355 | 1.00 | 41.61 | O |
| ATOM | 1715 | CB | ALA | A | 579 | −18.444 | −14.880 | −0.105 | 1.00 | 41.63 | C |
| ATOM | 1716 | N | GLN | A | 580 | −17.829 | −17.877 | 0.663 | 1.00 | 41.42 | N |
| ATOM | 1717 | CA | GLN | A | 580 | −17.752 | −19.318 | 0.445 | 1.00 | 41.20 | C |
| ATOM | 1718 | C | GLN | A | 580 | −18.525 | −20.079 | 1.520 | 1.00 | 41.06 | C |
| ATOM | 1719 | O | GLN | A | 580 | −19.458 | −20.819 | 1.209 | 1.00 | 40.94 | O |
| ATOM | 1720 | CB | GLN | A | 580 | −16.289 | −19.783 | 0.402 | 1.00 | 41.12 | C |
| ATOM | 1721 | CG | GLN | A | 580 | −15.590 | −19.520 | −0.919 | 1.00 | 41.33 | C |
| ATOM | 1722 | CD | GLN | A | 580 | −14.072 | −19.399 | −0.791 | 1.00 | 41.74 | C |
| ATOM | 1723 | OE1 | GLN | A | 580 | −13.539 | −19.132 | 0.291 | 1.00 | 41.10 | O |
| ATOM | 1724 | NE2 | GLN | A | 580 | −13.371 | −19.580 | −1.913 | 1.00 | 41.60 | N |
| ATOM | 1725 | N | GLU | A | 581 | −18.153 | −19.877 | 2.782 | 1.00 | 41.09 | N |
| ATOM | 1726 | CA | GLU | A | 581 | −18.779 | −20.605 | 3.881 | 1.00 | 41.19 | C |
| ATOM | 1727 | C | GLU | A | 581 | −20.304 | −20.450 | 3.888 | 1.00 | 41.01 | C |
| ATOM | 1728 | O | GLU | A | 581 | −21.017 | −21.409 | 4.160 | 1.00 | 40.92 | O |
| ATOM | 1729 | CB | GLU | A | 581 | −18.187 | −20.206 | 5.231 | 1.00 | 41.20 | C |
| ATOM | 1730 | CG | GLU | A | 581 | −18.230 | −21.346 | 6.245 | 1.00 | 42.30 | C |
| ATOM | 1731 | CD | GLU | A | 581 | −18.687 | −20.904 | 7.629 | 1.00 | 43.34 | C |
| ATOM | 1732 | OE1 | GLU | A | 581 | −19.622 | −21.533 | 8.173 | 1.00 | 42.31 | O |
| ATOM | 1733 | OE2 | GLU | A | 581 | −18.116 | −19.928 | 8.171 | 1.00 | 44.89 | O |
| ATOM | 1734 | N | ALA | A | 582 | −20.789 | −19.250 | 3.572 | 1.00 | 40.99 | N |
| ATOM | 1735 | CA | ALA | A | 582 | −22.225 | −18.996 | 3.445 | 1.00 | 40.98 | C |
| ATOM | 1736 | C | ALA | A | 582 | −22.879 | −19.876 | 2.379 | 1.00 | 41.09 | C |
| ATOM | 1737 | O | ALA | A | 582 | −23.933 | −20.467 | 2.633 | 1.00 | 40.97 | O |
| ATOM | 1738 | CB | ALA | A | 582 | −22.496 | −17.525 | 3.167 | 1.00 | 40.84 | C |
| ATOM | 1739 | N | LEU | A | 583 | −22.248 | −19.966 | 1.204 | 1.00 | 41.24 | N |
| ATOM | 1740 | CA | LEU | A | 583 | −22.733 | −20.821 | 0.114 | 1.00 | 41.53 | C |
| ATOM | 1741 | C | LEU | A | 583 | −22.777 | −22.295 | 0.518 | 1.00 | 41.76 | C |
| ATOM | 1742 | O | LEU | A | 583 | −23.717 | −23.013 | 0.170 | 1.00 | 41.88 | O |
| ATOM | 1743 | CB | LEU | A | 583 | −21.874 | −20.658 | −1.146 | 1.00 | 41.61 | C |
| ATOM | 1744 | CG | LEU | A | 583 | −21.980 | −19.416 | −2.042 | 1.00 | 41.59 | C |
| ATOM | 1745 | CD1 | LEU | A | 583 | −20.889 | −19.453 | −3.096 | 1.00 | 41.17 | C |
| ATOM | 1746 | CD2 | LEU | A | 583 | −23.340 | −19.303 | −2.708 | 1.00 | 41.79 | C |
| ATOM | 1747 | N | THR | A | 584 | −21.754 | −22.734 | 1.250 | 1.00 | 42.04 | N |
| ATOM | 1748 | CA | THR | A | 584 | −21.703 | −24.087 | 1.804 | 1.00 | 42.32 | C |
| ATOM | 1749 | C | THR | A | 584 | −22.857 | −24.331 | 2.788 | 1.00 | 42.60 | C |
| ATOM | 1750 | O | THR | A | 584 | −23.648 | −25.254 | 2.595 | 1.00 | 42.66 | O |
| ATOM | 1751 | CB | THR | A | 584 | −20.342 | −24.362 | 2.491 | 1.00 | 42.26 | C |
| ATOM | 1752 | OG1 | THR | A | 584 | −19.291 | −24.261 | 1.525 | 1.00 | 42.19 | O |
| ATOM | 1753 | CG2 | THR | A | 584 | −20.311 | −25.748 | 3.110 | 1.00 | 42.45 | C |
| ATOM | 1754 | N | VAL | A | 585 | −22.955 | −23.489 | 3.817 | 1.00 | 42.87 | N |
| ATOM | 1755 | CA | VAL | A | 585 | −23.982 | −23.615 | 4.865 | 1.00 | 43.18 | C |
| ATOM | 1756 | C | VAL | A | 585 | −25.420 | −23.526 | 4.314 | 1.00 | 43.52 | C |
| ATOM | 1757 | O | VAL | A | 585 | −26.357 | −24.063 | 4.917 | 1.00 | 43.54 | O |
| ATOM | 1758 | CB | VAL | A | 585 | −23.739 | −22.592 | 6.033 | 1.00 | 43.19 | C |
| ATOM | 1759 | CG1 | VAL | A | 585 | −24.911 | −22.555 | 7.017 | 1.00 | 43.06 | C |
| ATOM | 1760 | CG2 | VAL | A | 585 | −22.446 | −22.924 | 6.779 | 1.00 | 42.88 | C |
| ATOM | 1761 | N | LEU | A | 586 | −25.581 | −22.871 | 3.164 | 1.00 | 43.84 | N |
| ATOM | 1762 | CA | LEU | A | 586 | −26.895 | −22.729 | 2.523 | 1.00 | 44.07 | C |
| ATOM | 1763 | C | LEU | A | 586 | −27.152 | −23.777 | 1.430 | 1.00 | 44.16 | C |
| ATOM | 1764 | O | LEU | A | 586 | −28.078 | −23.630 | 0.625 | 1.00 | 44.14 | O |
| ATOM | 1765 | CB | LEU | A | 586 | −27.064 | −21.316 | 1.949 | 1.00 | 44.12 | C |
| ATOM | 1766 | CG | LEU | A | 586 | −27.059 | −20.093 | 2.871 | 1.00 | 44.20 | C |
| ATOM | 1767 | CD1 | LEU | A | 586 | −27.065 | −18.835 | 2.029 | 1.00 | 44.35 | C |
| ATOM | 1768 | CD2 | LEU | A | 586 | −28.239 | −20.095 | 3.832 | 1.00 | 44.62 | C |
| ATOM | 1769 | N | SER | A | 587 | −26.325 | −24.826 | 1.413 | 1.00 | 44.32 | N |
| ATOM | 1770 | CA | SER | A | 587 | −26.403 | −25.931 | 0.435 | 1.00 | 44.37 | C |
| ATOM | 1771 | C | SER | A | 587 | −26.260 | −25.497 | −1.025 | 1.00 | 44.47 | C |
| ATOM | 1772 | O | SER | A | 587 | −26.437 | −26.308 | −1.937 | 1.00 | 44.44 | O |
| ATOM | 1773 | CB | SER | A | 587 | −27.681 | −26.754 | 0.625 | 1.00 | 44.23 | C |
| ATOM | 1774 | OG | SER | A | 587 | −27.662 | −27.420 | 1.871 | 1.00 | 44.25 | O |
| ATOM | 1775 | N | LEU | A | 588 | −25.932 | −24.225 | −1.233 | 1.00 | 44.59 | N |
| ATOM | 1776 | CA | LEU | A | 588 | −25.805 | −23.662 | −2.571 | 1.00 | 44.82 | C |
| ATOM | 1777 | C | LEU | A | 588 | −24.504 | −24.110 | −3.236 | 1.00 | 45.05 | C |
| ATOM | 1778 | O | LEU | A | 588 | −24.527 | −24.617 | −4.363 | 1.00 | 45.20 | O |
| ATOM | 1779 | CB | LEU | A | 588 | −25.903 | −22.132 | −2.530 | 1.00 | 44.86 | C |
| ATOM | 1780 | CG | LEU | A | 588 | −27.204 | −21.494 | −2.025 | 1.00 | 44.68 | C |
| ATOM | 1781 | CD1 | LEU | A | 588 | −26.970 | −20.051 | −1.617 | 1.00 | 44.63 | C |
| ATOM | 1782 | CD2 | LEU | A | 588 | −28.302 | −21.571 | −3.066 | 1.00 | 44.67 | C |
| ATOM | 1783 | N | ALA | A | 589 | −23.386 | −23.924 | −2.528 | 1.00 | 45.19 | N |
| ATOM | 1784 | CA | ALA | A | 589 | −22.048 | −24.353 | −2.973 | 1.00 | 45.38 | C |
| ATOM | 1785 | C | ALA | A | 589 | −21.722 | −24.005 | −4.435 | 1.00 | 45.51 | C |
| ATOM | 1786 | O | ALA | A | 589 | −21.754 | −24.847 | −5.342 | 1.00 | 45.45 | O |
| ATOM | 1787 | CB | ALA | A | 589 | −21.835 | −25.854 | −2.699 | 1.00 | 45.40 | C |
| ATOM | 1788 | OXT | ALA | A | 589 | −21.414 | −22.850 | −4.739 | 1.00 | 45.67 | O |
| TER | 1789 | | ALA | A | 589 | | | | | | |
| ATOM | 1790 | N | PHE | C | 6 | −7.276 | −25.855 | 9.231 | 1.00 | 51.72 | N |
| ATOM | 1791 | CA | PHE | C | 6 | −6.423 | −24.991 | 10.098 | 1.00 | 51.66 | C |
| ATOM | 1792 | C | PHE | C | 6 | −7.144 | −23.699 | 10.478 | 1.00 | 51.43 | C |
| ATOM | 1793 | O | PHE | C | 6 | −6.965 | −23.188 | 11.587 | 1.00 | 51.53 | O |
| ATOM | 1794 | CB | PHE | C | 6 | −5.084 | −24.677 | 9.412 | 1.00 | 51.92 | C |
| ATOM | 1795 | CG | PHE | C | 6 | −4.055 | −24.068 | 10.332 | 1.00 | 52.24 | C |
| ATOM | 1796 | CD1 | PHE | C | 6 | −3.265 | −24.876 | 11.148 | 1.00 | 52.56 | C |
| ATOM | 1797 | CD2 | PHE | C | 6 | −3.876 | −22.686 | 10.384 | 1.00 | 52.90 | C |
| ATOM | 1798 | CE1 | PHE | C | 6 | −2.314 | −24.314 | 12.009 | 1.00 | 53.01 | C |
| ATOM | 1799 | CE2 | PHE | C | 6 | −2.928 | −22.110 | 11.244 | 1.00 | 53.11 | C |
| ATOM | 1800 | CZ | PHE | C | 6 | −2.145 | −22.927 | 12.057 | 1.00 | 52.87 | C |
| ATOM | 1801 | N | THR | C | 7 | −7.949 | −23.174 | 9.556 | 1.00 | 50.99 | N |
| ATOM | 1802 | CA | THR | C | 7 | −8.777 | −21.999 | 9.828 | 1.00 | 50.63 | C |
| ATOM | 1803 | C | THR | C | 7 | −9.729 | −22.266 | 10.996 | 1.00 | 50.51 | C |
| ATOM | 1804 | O | THR | C | 7 | −9.952 | −21.394 | 11.835 | 1.00 | 50.61 | O |
| ATOM | 1805 | CB | THR | C | 7 | −9.571 | −21.565 | 8.577 | 1.00 | 50.50 | C |
| ATOM | 1806 | OG1 | THR | C | 7 | −8.657 | −21.119 | 7.576 | 1.00 | 50.77 | O |
| ATOM | 1807 | CG2 | THR | C | 7 | −10.522 | −20.427 | 8.889 | 1.00 | 50.10 | C |
| ATOM | 1808 | N | LYS | C | 8 | −10.267 | −23.480 | 11.053 | 1.00 | 50.30 | N |
| ATOM | 1809 | CA | LYS | C | 8 | −11.242 | −23.837 | 12.078 | 1.00 | 50.13 | C |
| ATOM | 1810 | C | LYS | C | 8 | −10.592 | −24.281 | 13.389 | 1.00 | 49.96 | C |
| ATOM | 1811 | O | LYS | C | 8 | −11.209 | −24.205 | 14.451 | 1.00 | 49.87 | O |
| ATOM | 1812 | CB | LYS | C | 8 | −12.238 | −24.868 | 11.535 | 1.00 | 50.15 | C |
| ATOM | 1813 | CG | LYS | C | 8 | −13.368 | −24.218 | 10.737 | 1.00 | 50.20 | C |
| ATOM | 1814 | CD | LYS | C | 8 | −13.707 | −24.980 | 9.465 | 1.00 | 50.79 | C |
| ATOM | 1815 | CE | LYS | C | 8 | −14.636 | −24.158 | 8.576 | 1.00 | 51.08 | C |
| ATOM | 1816 | NZ | LYS | C | 8 | −15.038 | −24.856 | 7.320 | 1.00 | 51.00 | N |
| ATOM | 1817 | N | GLU | C | 9 | −9.343 | −24.728 | 13.310 | 1.00 | 49.87 | N |
| ATOM | 1818 | CA | GLU | C | 9 | −8.546 | −24.960 | 14.509 | 1.00 | 49.83 | C |
| ATOM | 1819 | C | GLU | C | 9 | −8.251 | −23.636 | 15.203 | 1.00 | 49.80 | C |
| ATOM | 1820 | O | GLU | C | 9 | −8.266 | −23.554 | 16.434 | 1.00 | 49.83 | O |
| ATOM | 1821 | CB | GLU | C | 9 | −7.242 | −25.683 | 14.175 | 1.00 | 49.81 | C |
| ATOM | 1822 | CG | GLU | C | 9 | −7.254 | −27.153 | 14.540 | 1.00 | 49.92 | C |
| ATOM | 1823 | CD | GLU | C | 9 | −5.888 | −27.795 | 14.440 | 1.00 | 50.20 | C |
| ATOM | 1824 | OE1 | GLU | C | 9 | −5.829 | −29.002 | 14.115 | 1.00 | 50.73 | O |
| ATOM | 1825 | OE2 | GLU | C | 9 | −4.876 | −27.099 | 14.683 | 1.00 | 49.78 | O |
| ATOM | 1826 | N | LEU | C | 10 | −7.989 | −22.608 | 14.394 | 1.00 | 49.74 | N |
| ATOM | 1827 | CA | LEU | C | 10 | −7.769 | −21.244 | 14.870 | 1.00 | 49.43 | C |
| ATOM | 1828 | C | LEU | C | 10 | −9.004 | −20.688 | 15.571 | 1.00 | 49.12 | C |
| ATOM | 1829 | O | LEU | C | 10 | −8.892 | −20.084 | 16.635 | 1.00 | 49.15 | O |
| ATOM | 1830 | CB | LEU | C | 10 | −7.373 | −20.338 | 13.704 | 1.00 | 49.62 | C |
| ATOM | 1831 | CG | LEU | C | 10 | −5.922 | −20.375 | 13.210 | 1.00 | 50.06 | C |
| ATOM | 1832 | CD1 | LEU | C | 10 | −5.841 | −19.959 | 11.746 | 1.00 | 50.36 | C |
| ATOM | 1833 | CD2 | LEU | C | 10 | −5.037 | −19.480 | 14.071 | 1.00 | 49.77 | C |
| ATOM | 1834 | N | ASP | C | 11 | −10.175 | −20.905 | 14.973 | 1.00 | 48.86 | N |
| ATOM | 1835 | CA | ASP | C | 11 | −11.451 | −20.500 | 15.564 | 1.00 | 48.70 | C |
| ATOM | 1836 | C | ASP | C | 11 | −11.673 | −21.177 | 16.913 | 1.00 | 48.52 | C |
| ATOM | 1837 | O | ASP | C | 11 | −12.156 | −20.553 | 17.861 | 1.00 | 48.46 | O |
| ATOM | 1838 | CB | ASP | C | 11 | −12.609 | −20.810 | 14.613 | 1.00 | 48.65 | C |
| ATOM | 1839 | CG | ASP | C | 11 | −12.547 | −20.001 | 13.330 | 1.00 | 49.02 | C |
| ATOM | 1840 | OD1 | ASP | C | 11 | −12.821 | −20.569 | 12.258 | 1.00 | 49.18 | O |
| ATOM | 1841 | OD2 | ASP | C | 11 | −12.219 | −18.796 | 13.383 | 1.00 | 50.32 | O |
| ATOM | 1842 | N | GLN | C | 12 | −11.303 | −22.453 | 16.984 | 1.00 | 48.31 | N |
| ATOM | 1843 | CA | GLN | C | 12 | −11.357 | −23.218 | 18.223 | 1.00 | 48.11 | C |
| ATOM | 1844 | C | GLN | C | 12 | −10.437 | −22.597 | 19.278 | 1.00 | 47.95 | C |
| ATOM | 1845 | O | GLN | C | 12 | −10.854 | −22.390 | 20.418 | 1.00 | 48.00 | O |
| ATOM | 1846 | CB | GLN | C | 12 | −10.994 | −24.685 | 17.961 | 1.00 | 48.07 | C |
| ATOM | 1847 | CG | GLN | C | 12 | −11.392 | −25.645 | 19.069 | 1.00 | 48.46 | C |
| ATOM | 1848 | CD | GLN | C | 12 | −10.501 | −25.535 | 20.294 | 1.00 | 49.43 | C |
| ATOM | 1849 | OE1 | GLN | C | 12 | −9.283 | −25.711 | 20.212 | 1.00 | 50.22 | O |
| ATOM | 1850 | NE2 | GLN | C | 12 | −11.105 | −25.233 | 21.439 | 1.00 | 49.39 | N |
| ATOM | 1851 | N | TRP | C | 13 | −9.198 | −22.289 | 18.893 | 1.00 | 47.65 | N |
| ATOM | 1852 | CA | TRP | C | 13 | −8.242 | −21.665 | 19.808 | 1.00 | 47.36 | C |
| ATOM | 1853 | C | TRP | C | 13 | −8.738 | −20.307 | 20.313 | 1.00 | 47.22 | C |
| ATOM | 1854 | O | TRP | C | 13 | −8.611 | −20.000 | 21.504 | 1.00 | 47.36 | O |
| ATOM | 1855 | CB | TRP | C | 13 | −6.851 | −21.521 | 19.171 | 1.00 | 47.35 | C |
| ATOM | 1856 | CG | TRP | C | 13 | −6.186 | −22.817 | 18.749 | 1.00 | 47.60 | C |
| ATOM | 1857 | CD1 | TRP | C | 13 | −6.584 | −24.099 | 19.055 | 1.00 | 47.80 | C |
| ATOM | 1858 | CD2 | TRP | C | 13 | −4.988 | −22.951 | 17.968 | 1.00 | 47.76 | C |
| ATOM | 1859 | NE1 | TRP | C | 13 | −5.719 | −25.013 | 18.497 | 1.00 | 47.31 | N |
| ATOM | 1860 | CE2 | TRP | C | 13 | −4.731 | −24.340 | 17.827 | 1.00 | 47.65 | C |
| ATOM | 1861 | CE3 | TRP | C | 13 | −4.111 | −22.036 | 17.368 | 1.00 | 47.52 | C |
| ATOM | 1862 | CZ2 | TRP | C | 13 | −3.629 | −24.832 | 17.113 | 1.00 | 47.29 | C |
| ATOM | 1863 | CZ3 | TRP | C | 13 | −3.016 | −22.527 | 16.655 | 1.00 | 47.56 | C |
| ATOM | 1864 | CH2 | TRP | C | 13 | −2.789 | −23.914 | 16.534 | 1.00 | 47.53 | C |
| ATOM | 1865 | N | ILE | C | 14 | −9.309 | −19.506 | 19.413 | 1.00 | 46.84 | N |
| ATOM | 1866 | CA | ILE | C | 14 | −9.836 | −18.184 | 19.775 | 1.00 | 46.60 | C |
| ATOM | 1867 | C | ILE | C | 14 | −10.987 | −18.318 | 20.786 | 1.00 | 46.60 | C |
| ATOM | 1868 | O | ILE | C | 14 | −10.935 | −17.742 | 21.875 | 1.00 | 46.69 | O |
| ATOM | 1869 | CB | ILE | C | 14 | −10.238 | −17.355 | 18.522 | 1.00 | 46.48 | C |
| ATOM | 1870 | CG1 | ILE | C | 14 | −8.991 | −16.981 | 17.716 | 1.00 | 46.00 | C |
| ATOM | 1871 | CG2 | ILE | C | 14 | −10.996 | −16.089 | 18.910 | 1.00 | 45.90 | C |
| ATOM | 1872 | CD1 | ILE | C | 14 | −9.256 | −16.743 | 16.236 | 1.00 | 45.57 | C |
| ATOM | 1873 | N | GLU | C | 15 | −11.999 | −19.105 | 20.432 | 1.00 | 46.46 | N |
| ATOM | 1874 | CA | GLU | C | 15 | −13.091 | −19.438 | 21.342 | 1.00 | 46.41 | C |
| ATOM | 1875 | C | GLU | C | 15 | −12.570 | −19.796 | 22.741 | 1.00 | 46.14 | C |
| ATOM | 1876 | O | GLU | C | 15 | −13.142 | −19.377 | 23.748 | 1.00 | 46.26 | O |
| ATOM | 1877 | CB | GLU | C | 15 | −13.914 | −20.598 | 20.770 | 1.00 | 46.38 | C |
| ATOM | 1878 | CG | GLU | C | 15 | −15.174 | −20.947 | 21.561 | 1.00 | 47.47 | C |
| ATOM | 1879 | CD | GLU | C | 15 | −15.521 | −22.438 | 21.503 | 1.00 | 49.17 | C |
| ATOM | 1880 | OE1 | GLU | C | 15 | −14.720 | −23.264 | 22.001 | 1.00 | 49.55 | O |
| ATOM | 1881 | OE2 | GLU | C | 15 | −16.601 | −22.787 | 20.971 | 1.00 | 49.61 | O |
| ATOM | 1882 | N | GLN | C | 16 | −11.479 | −20.558 | 22.789 | 1.00 | 45.78 | N |
| ATOM | 1883 | CA | GLN | C | 16 | −10.915 | −21.053 | 24.041 | 1.00 | 45.54 | C |
| ATOM | 1884 | C | GLN | C | 16 | −10.218 | −19.944 | 24.827 | 1.00 | 45.47 | C |
| ATOM | 1885 | O | GLN | C | 16 | −10.426 | −19.790 | 26.033 | 1.00 | 45.42 | O |
| ATOM | 1886 | CB | GLN | C | 16 | −9.939 | −22.197 | 23.757 | 1.00 | 45.57 | C |
| ATOM | 1887 | CG | GLN | C | 16 | −9.469 | −22.964 | 24.989 | 1.00 | 45.46 | C |
| ATOM | 1888 | CD | GLN | C | 16 | −8.278 | −23.859 | 24.694 | 1.00 | 45.47 | C |
| ATOM | 1889 | OE1 | GLN | C | 16 | −8.394 | −24.856 | 23.985 | 1.00 | 45.72 | O |
| ATOM | 1890 | NE2 | GLN | C | 16 | −7.126 | −23.503 | 25.241 | 1.00 | 45.70 | N |
| ATOM | 1891 | N | LEU | C | 17 | −9.390 | −19.173 | 24.137 | 1.00 | 45.40 | N |
| ATOM | 1892 | CA | LEU | C | 17 | −8.682 | −18.064 | 24.758 | 1.00 | 45.34 | C |
| ATOM | 1893 | C | LEU | C | 17 | −9.649 | −17.009 | 25.279 | 1.00 | 45.42 | C |
| ATOM | 1894 | O | LEU | C | 17 | −9.356 | −16.333 | 26.266 | 1.00 | 45.73 | O |
| ATOM | 1895 | CB | LEU | C | 17 | −7.700 | −17.439 | 23.769 | 1.00 | 45.33 | C |
| ATOM | 1896 | CG | LEU | C | 17 | −6.439 | −18.255 | 23.482 | 1.00 | 44.74 | C |
| ATOM | 1897 | CD1 | LEU | C | 17 | −5.932 | −17.982 | 22.070 | 1.00 | 43.75 | C |
| ATOM | 1898 | CD2 | LEU | C | 17 | −5.369 | −17.989 | 24.537 | 1.00 | 43.16 | C |
| ATOM | 1899 | N | ASN | C | 18 | −10.800 | −16.877 | 24.618 | 1.00 | 45.28 | N |
| ATOM | 1900 | CA | ASN | C | 18 | −11.877 | −15.998 | 25.084 | 1.00 | 45.18 | C |
| ATOM | 1901 | C | ASN | C | 18 | −12.425 | −16.389 | 26.457 | 1.00 | 45.11 | C |
| ATOM | 1902 | O | ASN | C | 18 | −13.078 | −15.597 | 27.128 | 1.00 | 44.81 | O |
| ATOM | 1903 | CB | ASN | C | 18 | −13.007 | −15.946 | 24.057 | 1.00 | 45.10 | C |
| ATOM | 1904 | CG | ASN | C | 18 | −12.794 | −14.867 | 23.023 | 1.00 | 45.05 | C |
| ATOM | 1905 | OD1 | ASN | C | 18 | −13.017 | −13.688 | 23.281 | 1.00 | 44.95 | O |
| ATOM | 1906 | ND2 | ASN | C | 18 | −12.355 | −15.265 | 21.845 | 1.00 | 45.80 | N |
| ATOM | 1907 | N | GLU | C | 19 | −12.144 | −17.621 | 26.861 | 1.00 | 45.33 | N |
| ATOM | 1908 | CA | GLU | C | 19 | −12.579 | −18.131 | 28.145 | 1.00 | 45.74 | C |
| ATOM | 1909 | C | GLU | C | 19 | −11.396 | −18.161 | 29.107 | 1.00 | 45.44 | C |
| ATOM | 1910 | O | GLU | C | 19 | −11.433 | −18.860 | 30.116 | 1.00 | 45.76 | O |
| ATOM | 1911 | CB | GLU | C | 19 | −13.175 | −19.532 | 27.979 | 1.00 | 45.64 | C |
| ATOM | 1912 | CG | GLU | C | 19 | −14.421 | −19.599 | 27.102 | 1.00 | 46.04 | C |
| ATOM | 1913 | CD | GLU | C | 19 | −15.012 | −21.004 | 27.028 | 1.00 | 47.08 | C |
| ATOM | 1914 | OE1 | GLU | C | 19 | −15.060 | −21.571 | 25.912 | 1.00 | 49.45 | O |
| ATOM | 1915 | OE2 | GLU | C | 19 | −15.430 | −21.552 | 28.081 | 1.00 | 49.29 | O |
| ATOM | 1916 | N | CYS | C | 20 | −10.358 | −17.387 | 28.790 | 1.00 | 44.94 | N |
| ATOM | 1917 | CA | CYS | C | 20 | −9.091 | −17.385 | 29.531 | 1.00 | 44.75 | C |
| ATOM | 1918 | C | CYS | C | 20 | −8.506 | −18.773 | 29.802 | 1.00 | 44.69 | C |
| ATOM | 1919 | O | CYS | C | 20 | −8.143 | −19.089 | 30.938 | 1.00 | 44.76 | O |
| ATOM | 1920 | CB | CYS | C | 20 | −9.204 | −16.613 | 30.845 | 1.00 | 44.65 | C |
| ATOM | 1921 | SG | CYS | C | 20 | −7.586 | −16.065 | 31.451 | 1.00 | 44.50 | S |
| ATOM | 1922 | N | LYS | C | 21 | −8.415 | −19.592 | 28.759 | 1.00 | 44.53 | N |
| ATOM | 1923 | CA | LYS | C | 21 | −7.831 | −20.925 | 28.873 | 1.00 | 44.47 | C |
| ATOM | 1924 | C | LYS | C | 21 | −6.686 | −21.088 | 27.879 | 1.00 | 44.10 | C |
| ATOM | 1925 | O | LYS | C | 21 | −6.899 | −21.099 | 26.662 | 1.00 | 44.27 | O |
| ATOM | 1926 | CB | LYS | C | 21 | −8.898 | −22.003 | 28.678 | 1.00 | 44.56 | C |
| ATOM | 1927 | CG | LYS | C | 21 | −9.931 | −22.037 | 29.796 | 1.00 | 45.69 | C |
| ATOM | 1928 | CD | LYS | C | 21 | −11.177 | −22.802 | 29.375 | 1.00 | 47.78 | C |
| ATOM | 1929 | CE | LYS | C | 21 | −12.286 | −22.684 | 30.418 | 1.00 | 48.48 | C |
| ATOM | 1930 | NZ | LYS | C | 21 | −13.494 | −23.481 | 30.019 | 1.00 | 49.19 | N |
| ATOM | 1931 | N | GLN | C | 22 | −5.473 | −21.201 | 28.417 | 1.00 | 43.54 | N |
| ATOM | 1932 | CA | GLN | C | 22 | −4.244 | −21.264 | 27.621 | 1.00 | 42.79 | C |
| ATOM | 1933 | C | GLN | C | 22 | −4.269 | −22.375 | 26.572 | 1.00 | 42.65 | C |
| ATOM | 1934 | O | GLN | C | 22 | −4.918 | −23.410 | 26.758 | 1.00 | 42.50 | O |
| ATOM | 1935 | CB | GLN | C | 22 | −3.026 | −21.456 | 28.538 | 1.00 | 42.77 | C |
| ATOM | 1936 | CG | GLN | C | 22 | −2.913 | −22.855 | 29.155 | 1.00 | 42.20 | C |
| ATOM | 1937 | CD | GLN | C | 22 | −1.853 | −22.957 | 30.228 | 1.00 | 42.18 | C |
| ATOM | 1938 | OE1 | GLN | C | 22 | −1.866 | −22.209 | 31.199 | 1.00 | 42.31 | O |
| ATOM | 1939 | NE2 | GLN | C | 22 | −0.939 | −23.900 | 30.067 | 1.00 | 41.22 | N |
| ATOM | 1940 | N | LEU | C | 23 | −3.552 | −22.150 | 25.475 | 1.00 | 42.32 | N |
| ATOM | 1941 | CA | LEU | C | 23 | −3.274 | −23.209 | 24.509 | 1.00 | 41.91 | C |
| ATOM | 1942 | C | LEU | C | 23 | −2.125 | −24.082 | 25.004 | 1.00 | 41.68 | C |
| ATOM | 1943 | O | LEU | C | 23 | −1.344 | −23.670 | 25.856 | 1.00 | 41.41 | O |
| ATOM | 1944 | CB | LEU | C | 23 | −2.949 | −22.618 | 23.133 | 1.00 | 41.86 | C |
| ATOM | 1945 | CG | LEU | C | 23 | −3.934 | −21.602 | 22.542 | 1.00 | 41.48 | C |
| ATOM | 1946 | CD1 | LEU | C | 23 | −3.471 | −21.182 | 21.167 | 1.00 | 41.40 | C |
| ATOM | 1947 | CD2 | LEU | C | 23 | −5.357 | −22.152 | 22.476 | 1.00 | 41.29 | C |
| ATOM | 1948 | N | SER | C | 24 | −2.038 | −25.299 | 24.479 | 1.00 | 41.86 | N |
| ATOM | 1949 | CA | SER | C | 24 | −0.945 | −26.206 | 24.814 | 1.00 | 41.85 | C |
| ATOM | 1950 | C | SER | C | 24 | 0.374 | −25.675 | 24.264 | 1.00 | 42.03 | C |
| ATOM | 1951 | O | SER | C | 24 | 0.390 | −24.884 | 23.311 | 1.00 | 42.21 | O |
| ATOM | 1952 | CB | SER | C | 24 | −1.213 | −27.567 | 24.209 | 1.00 | 41.78 | C |
| ATOM | 1953 | OG | SER | C | 24 | −1.134 | −27.482 | 22.801 | 1.00 | 41.83 | O |
| ATOM | 1954 | N | GLU | C | 25 | 1.478 | −26.117 | 24.858 | 1.00 | 42.03 | N |
| ATOM | 1955 | CA | GLU | C | 25 | 2.809 | −25.750 | 24.379 | 1.00 | 42.12 | C |
| ATOM | 1956 | C | GLU | C | 25 | 2.949 | −26.031 | 22.882 | 1.00 | 42.35 | C |
| ATOM | 1957 | O | GLU | C | 25 | 3.497 | −25.220 | 22.134 | 1.00 | 42.19 | O |
| ATOM | 1958 | CB | GLU | C | 25 | 3.881 | −26.514 | 25.157 | 1.00 | 41.99 | C |
| ATOM | 1959 | CG | GLU | C | 25 | 5.302 | −26.154 | 24.769 | 1.00 | 41.97 | C |
| ATOM | 1960 | CD | GLU | C | 25 | 6.320 | −27.119 | 25.323 | 1.00 | 42.44 | C |
| ATOM | 1961 | OE1 | GLU | C | 25 | 6.086 | −27.676 | 26.420 | 1.00 | 42.33 | O |
| ATOM | 1962 | OE2 | GLU | C | 25 | 7.358 | −27.315 | 24.656 | 1.00 | 42.50 | O |
| ATOM | 1963 | N | SER | C | 26 | 2.439 | −27.187 | 22.461 | 1.00 | 42.74 | N |
| ATOM | 1964 | CA | SER | C | 26 | 2.496 | −27.611 | 21.072 | 1.00 | 42.94 | C |
| ATOM | 1965 | C | SER | C | 26 | 1.789 | −26.617 | 20.157 | 1.00 | 43.00 | C |
| ATOM | 1966 | O | SER | C | 26 | 2.283 | −26.306 | 19.073 | 1.00 | 43.15 | O |
| ATOM | 1967 | CB | SER | C | 26 | 1.872 | −28.993 | 20.923 | 1.00 | 42.96 | C |
| ATOM | 1968 | OG | SER | C | 26 | 2.069 | −29.486 | 19.614 | 1.00 | 43.63 | O |
| ATOM | 1969 | N | GLN | C | 27 | 0.645 | −26.110 | 20.610 | 1.00 | 42.95 | N |
| ATOM | 1970 | CA | GLN | C | 27 | −0.179 | −25.214 | 19.807 | 1.00 | 42.86 | C |
| ATOM | 1971 | C | GLN | C | 27 | 0.424 | −23.828 | 19.702 | 1.00 | 42.83 | C |
| ATOM | 1972 | O | GLN | C | 27 | 0.335 | −23.181 | 18.658 | 1.00 | 42.79 | O |
| ATOM | 1973 | CB | GLN | C | 27 | −1.590 | −25.131 | 20.378 | 1.00 | 42.92 | C |
| ATOM | 1974 | CG | GLN | C | 27 | −2.411 | −26.391 | 20.143 | 1.00 | 42.37 | C |
| ATOM | 1975 | CD | GLN | C | 27 | −3.700 | −26.402 | 20.925 | 1.00 | 41.36 | C |
| ATOM | 1976 | OE1 | GLN | C | 27 | −3.829 | −25.740 | 21.954 | 1.00 | 41.40 | O |
| ATOM | 1977 | NE2 | GLN | C | 27 | −4.667 | −27.159 | 20.441 | 1.00 | 41.58 | N |
| ATOM | 1978 | N | VAL | C | 28 | 1.040 | −23.376 | 20.785 | 1.00 | 43.02 | N |
| ATOM | 1979 | CA | VAL | C | 28 | 1.711 | −22.077 | 20.795 | 1.00 | 43.10 | C |
| ATOM | 1980 | C | VAL | C | 28 | 2.828 | −22.083 | 19.753 | 1.00 | 43.18 | C |
| ATOM | 1981 | O | VAL | C | 28 | 2.983 | −21.130 | 18.996 | 1.00 | 43.34 | O |
| ATOM | 1982 | CB | VAL | C | 28 | 2.219 | −21.707 | 22.213 | 1.00 | 42.95 | C |
| ATOM | 1983 | CG1 | VAL | C | 28 | 3.057 | −20.445 | 22.186 | 1.00 | 43.25 | C |
| ATOM | 1984 | CG2 | VAL | C | 28 | 1.039 | −21.523 | 23.161 | 1.00 | 42.62 | C |
| ATOM | 1985 | N | LYS | C | 29 | 3.558 | −23.192 | 19.694 | 1.00 | 43.50 | N |
| ATOM | 1986 | CA | LYS | C | 29 | 4.654 | −23.387 | 18.747 | 1.00 | 43.79 | C |
| ATOM | 1987 | C | LYS | C | 29 | 4.216 | −23.180 | 17.294 | 1.00 | 43.90 | C |
| ATOM | 1988 | O | LYS | C | 29 | 4.897 | −22.494 | 16.534 | 1.00 | 44.06 | O |
| ATOM | 1989 | CB | LYS | C | 29 | 5.268 | −24.783 | 18.933 | 1.00 | 43.68 | C |
| ATOM | 1990 | CG | LYS | C | 29 | 6.642 | −24.973 | 18.307 | 1.00 | 43.68 | C |
| ATOM | 1991 | CD | LYS | C | 29 | 7.075 | −26.435 | 18.383 | 1.00 | 43.92 | C |
| ATOM | 1992 | CE | LYS | C | 29 | 8.382 | −26.690 | 17.638 | 1.00 | 44.23 | C |
| ATOM | 1993 | NZ | LYS | C | 29 | 9.531 | −25.979 | 18.271 | 1.00 | 44.56 | N |
| ATOM | 1994 | N | SER | C | 30 | 3.083 | −23.770 | 16.920 | 1.00 | 44.10 | N |
| ATOM | 1995 | CA | SER | C | 30 | 2.561 | −23.667 | 15.553 | 1.00 | 44.24 | C |
| ATOM | 1996 | C | SER | C | 30 | 2.079 | −22.248 | 15.266 | 1.00 | 44.44 | C |
| ATOM | 1997 | O | SER | C | 30 | 2.357 | −21.683 | 14.205 | 1.00 | 44.51 | O |
| ATOM | 1998 | CB | SER | C | 30 | 1.415 | −24.661 | 15.333 | 1.00 | 44.10 | C |
| ATOM | 1999 | OG | SER | C | 30 | 1.802 | −25.979 | 15.672 | 1.00 | 43.79 | O |
| ATOM | 2000 | N | LEU | C | 31 | 1.357 | −21.680 | 16.225 | 1.00 | 44.60 | N |
| ATOM | 2001 | CA | LEU | C | 31 | 0.863 | −20.319 | 16.114 | 1.00 | 44.97 | C |
| ATOM | 2002 | C | LEU | C | 31 | 2.008 | −19.372 | 15.766 | 1.00 | 45.20 | C |
| ATOM | 2003 | O | LEU | C | 31 | 1.932 | −18.626 | 14.788 | 1.00 | 45.18 | O |
| ATOM | 2004 | CB | LEU | C | 31 | 0.185 | −19.898 | 17.425 | 1.00 | 44.90 | C |
| ATOM | 2005 | CG | LEU | C | 31 | −0.745 | −18.687 | 17.370 | 1.00 | 44.84 | C |
| ATOM | 2006 | CD1 | LEU | C | 31 | −1.755 | −18.821 | 16.237 | 1.00 | 44.43 | C |
| ATOM | 2007 | CD2 | LEU | C | 31 | −1.449 | −18.508 | 18.701 | 1.00 | 44.99 | C |
| ATOM | 2008 | N | CYS | C | 32 | 3.073 | −19.445 | 16.562 | 1.00 | 45.45 | N |
| ATOM | 2009 | CA | CYS | C | 32 | 4.251 | −18.609 | 16.406 | 1.00 | 45.71 | C |
| ATOM | 2010 | C | CYS | C | 32 | 4.985 | −18.856 | 15.098 | 1.00 | 45.95 | C |
| ATOM | 2011 | O | CYS | C | 32 | 5.584 | −17.943 | 14.544 | 1.00 | 46.12 | O |
| ATOM | 2012 | CB | CYS | C | 32 | 5.203 | −18.818 | 17.584 | 1.00 | 45.73 | C |
| ATOM | 2013 | SG | CYS | C | 32 | 4.572 | −18.176 | 19.158 | 1.00 | 45.82 | S |
| ATOM | 2014 | N | GLU | C | 33 | 4.946 | −20.088 | 14.606 | 1.00 | 46.40 | N |
| ATOM | 2015 | CA | GLU | C | 33 | 5.604 | −20.406 | 13.343 | 1.00 | 46.91 | C |
| ATOM | 2016 | C | GLU | C | 33 | 4.937 | −19.662 | 12.198 | 1.00 | 46.99 | C |
| ATOM | 2017 | O | GLU | C | 33 | 5.616 | −19.080 | 11.353 | 1.00 | 47.04 | O |
| ATOM | 2018 | CB | GLU | C | 33 | 5.614 | −21.913 | 13.083 | 1.00 | 46.66 | C |
| ATOM | 2019 | CG | GLU | C | 33 | 6.737 | −22.661 | 13.795 | 1.00 | 47.08 | C |
| ATOM | 2020 | CD | GLU | C | 33 | 6.569 | −24.178 | 13.743 | 1.00 | 47.65 | C |
| ATOM | 2021 | OE1 | GLU | C | 33 | 7.066 | −24.870 | 14.661 | 1.00 | 48.68 | O |
| ATOM | 2022 | OE2 | GLU | C | 33 | 5.938 | −24.688 | 12.788 | 1.00 | 48.71 | O |
| ATOM | 2023 | N | LYS | C | 34 | 3.607 | −19.662 | 12.188 | 1.00 | 47.22 | N |
| ATOM | 2024 | CA | LYS | C | 34 | 2.865 | −19.023 | 11.113 | 1.00 | 47.50 | C |
| ATOM | 2025 | C | LYS | C | 34 | 2.840 | −17.507 | 11.264 | 1.00 | 47.40 | C |
| ATOM | 2026 | O | LYS | C | 34 | 2.783 | −16.785 | 10.269 | 1.00 | 47.48 | O |
| ATOM | 2027 | CB | LYS | C | 34 | 1.449 | −19.595 | 10.990 | 1.00 | 47.58 | C |
| ATOM | 2028 | CG | LYS | C | 34 | 0.880 | −19.433 | 9.588 | 1.00 | 48.42 | C |
| ATOM | 2029 | CD | LYS | C | 34 | −0.131 | −20.521 | 9.244 | 1.00 | 50.20 | C |
| ATOM | 2030 | CE | LYS | C | 34 | −0.194 | −20.771 | 7.733 | 1.00 | 50.65 | C |
| ATOM | 2031 | NZ | LYS | C | 34 | −0.378 | −19.513 | 6.947 | 1.00 | 50.87 | N |
| ATOM | 2032 | N | ALA | C | 35 | 2.887 | −17.035 | 12.508 | 1.00 | 47.38 | N |
| ATOM | 2033 | CA | ALA | C | 35 | 2.940 | −15.601 | 12.797 | 1.00 | 47.31 | C |
| ATOM | 2034 | C | ALA | C | 35 | 4.226 | −14.981 | 12.269 | 1.00 | 47.27 | C |
| ATOM | 2035 | O | ALA | C | 35 | 4.190 | −13.910 | 11.672 | 1.00 | 47.23 | O |
| ATOM | 2036 | CB | ALA | C | 35 | 2.792 | −15.339 | 14.294 | 1.00 | 47.34 | C |
| ATOM | 2037 | N | LYS | C | 36 | 5.352 | −15.662 | 12.486 | 1.00 | 47.31 | N |
| ATOM | 2038 | CA | LYS | C | 36 | 6.637 | −15.257 | 11.918 | 1.00 | 47.59 | C |
| ATOM | 2039 | C | LYS | C | 36 | 6.523 | −15.083 | 10.406 | 1.00 | 47.55 | C |
| ATOM | 2040 | O | LYS | C | 36 | 6.972 | −14.082 | 9.846 | 1.00 | 47.64 | O |
| ATOM | 2041 | CB | LYS | C | 36 | 7.708 | −16.304 | 12.212 | 1.00 | 47.40 | C |
| ATOM | 2042 | CG | LYS | C | 36 | 8.261 | −16.304 | 13.628 | 1.00 | 48.03 | C |
| ATOM | 2043 | CD | LYS | C | 36 | 9.242 | −17.472 | 13.797 | 1.00 | 48.35 | C |
| ATOM | 2044 | CE | LYS | C | 36 | 9.781 | −17.592 | 15.217 | 1.00 | 49.62 | C |
| ATOM | 2045 | NZ | LYS | C | 36 | 10.596 | −18.836 | 15.381 | 1.00 | 49.87 | N |
| ATOM | 2046 | N | GLU | C | 37 | 5.895 | −16.067 | 9.768 | 1.00 | 47.59 | N |
| ATOM | 2047 | CA | GLU | C | 37 | 5.739 | −16.130 | 8.320 | 1.00 | 47.56 | C |
| ATOM | 2048 | C | GLU | C | 37 | 4.930 | −14.957 | 7.763 | 1.00 | 47.29 | C |
| ATOM | 2049 | O | GLU | C | 37 | 5.257 | −14.418 | 6.700 | 1.00 | 47.43 | O |
| ATOM | 2050 | CB | GLU | C | 37 | 5.060 | −17.443 | 7.955 | 1.00 | 47.51 | C |
| ATOM | 2051 | CG | GLU | C | 37 | 5.821 | −18.263 | 6.958 | 1.00 | 48.70 | C |
| ATOM | 2052 | CD | GLU | C | 37 | 5.462 | −19.736 | 7.026 | 1.00 | 50.41 | C |
| ATOM | 2053 | OE1 | GLU | C | 37 | 4.273 | −20.055 | 7.256 | 1.00 | 51.19 | O |
| ATOM | 2054 | OE2 | GLU | C | 37 | 6.371 | −20.577 | 6.842 | 1.00 | 50.92 | O |
| ATOM | 2055 | N | ILE | C | 38 | 3.877 | −14.576 | 8.483 | 1.00 | 46.76 | N |
| ATOM | 2056 | CA | ILE | C | 38 | 3.043 | −13.438 | 8.107 | 1.00 | 46.32 | C |
| ATOM | 2057 | C | ILE | C | 38 | 3.798 | −12.119 | 8.303 | 1.00 | 46.16 | C |
| ATOM | 2058 | O | ILE | C | 38 | 3.887 | −11.297 | 7.386 | 1.00 | 46.09 | O |
| ATOM | 2059 | CB | ILE | C | 38 | 1.710 | −13.428 | 8.904 | 1.00 | 46.33 | C |
| ATOM | 2060 | CG1 | ILE | C | 38 | 0.828 | −14.608 | 8.472 | 1.00 | 46.18 | C |
| ATOM | 2061 | CG2 | ILE | C | 38 | 0.977 | −12.089 | 8.733 | 1.00 | 46.03 | C |
| ATOM | 2062 | CD1 | ILE | C | 38 | −0.372 | −14.879 | 9.372 | 1.00 | 46.03 | C |
| ATOM | 2063 | N | LEU | C | 39 | 4.352 | −11.945 | 9.499 | 1.00 | 45.98 | N |
| ATOM | 2064 | CA | LEU | C | 39 | 5.034 | −10.718 | 9.897 | 1.00 | 45.76 | C |
| ATOM | 2065 | C | LEU | C | 39 | 6.316 | −10.431 | 9.111 | 1.00 | 45.71 | C |
| ATOM | 2066 | O | LEU | C | 39 | 6.753 | −9.285 | 9.050 | 1.00 | 45.77 | O |
| ATOM | 2067 | CB | LEU | C | 39 | 5.320 | −10.748 | 11.399 | 1.00 | 45.78 | C |
| ATOM | 2068 | CG | LEU | C | 39 | 4.112 | −10.649 | 12.340 | 1.00 | 45.96 | C |
| ATOM | 2069 | CD1 | LEU | C | 39 | 4.421 | −11.263 | 13.707 | 1.00 | 45.67 | C |
| ATOM | 2070 | CD2 | LEU | C | 39 | 3.626 | −9.207 | 12.478 | 1.00 | 45.74 | C |
| ATOM | 2071 | N | THR | C | 40 | 6.913 | −11.469 | 8.527 | 1.00 | 45.61 | N |
| ATOM | 2072 | CA | THR | C | 40 | 8.053 | −11.329 | 7.621 | 1.00 | 45.73 | C |
| ATOM | 2073 | C | THR | C | 40 | 7.714 | −10.431 | 6.429 | 1.00 | 45.76 | C |
| ATOM | 2074 | O | THR | C | 40 | 8.536 | −9.622 | 5.993 | 1.00 | 45.64 | O |
| ATOM | 2075 | CB | THR | C | 40 | 8.500 | −12.709 | 7.099 | 1.00 | 45.84 | C |
| ATOM | 2076 | OG1 | THR | C | 40 | 8.809 | −13.551 | 8.212 | 1.00 | 46.59 | O |
| ATOM | 2077 | CG2 | THR | C | 40 | 9.730 | −12.601 | 6.201 | 1.00 | 45.63 | C |
| ATOM | 2078 | N | LYS | C | 41 | 6.495 | −10.583 | 5.918 | 1.00 | 45.88 | N |
| ATOM | 2079 | CA | LYS | C | 41 | 5.996 | −9.798 | 4.795 | 1.00 | 45.98 | C |
| ATOM | 2080 | C | LYS | C | 41 | 5.675 | −8.368 | 5.200 | 1.00 | 45.79 | C |
| ATOM | 2081 | O | LYS | C | 41 | 5.451 | −7.513 | 4.353 | 1.00 | 45.93 | O |
| ATOM | 2082 | CB | LYS | C | 41 | 4.747 | −10.456 | 4.198 | 1.00 | 46.03 | C |
| ATOM | 2083 | CG | LYS | C | 41 | 5.027 | −11.589 | 3.220 | 1.00 | 46.15 | C |
| ATOM | 2084 | CD | LYS | C | 41 | 3.725 | −12.206 | 2.722 | 1.00 | 46.58 | C |
| ATOM | 2085 | CE | LYS | C | 41 | 3.964 | −13.241 | 1.622 | 1.00 | 47.82 | C |
| ATOM | 2086 | NZ | LYS | C | 41 | 2.736 | −14.040 | 1.325 | 1.00 | 47.47 | N |
| ATOM | 2087 | N | GLU | C | 42 | 5.651 | −8.099 | 6.495 | 1.00 | 45.78 | N |
| ATOM | 2088 | CA | GLU | C | 42 | 5.319 | −6.759 | 6.959 | 1.00 | 45.82 | C |
| ATOM | 2089 | C | GLU | C | 42 | 6.543 | −5.842 | 7.036 | 1.00 | 45.46 | C |
| ATOM | 2090 | O | GLU | C | 42 | 7.678 | −6.302 | 7.201 | 1.00 | 45.22 | O |
| ATOM | 2091 | CB | GLU | C | 42 | 4.564 | −6.820 | 8.289 | 1.00 | 46.14 | C |
| ATOM | 2092 | CG | GLU | C | 42 | 3.191 | −7.464 | 8.165 | 1.00 | 47.02 | C |
| ATOM | 2093 | CD | GLU | C | 42 | 2.201 | −6.968 | 9.201 | 1.00 | 49.65 | C |
| ATOM | 2094 | OE1 | GLU | C | 42 | 2.493 | −5.983 | 9.918 | 1.00 | 50.23 | O |
| ATOM | 2095 | OE2 | GLU | C | 42 | 1.108 | −7.564 | 9.291 | 1.00 | 51.15 | O |
| ATOM | 2096 | N | SER | C | 43 | 6.290 | −4.543 | 6.908 | 1.00 | 45.09 | N |
| ATOM | 2097 | CA | SER | C | 43 | 7.333 | −3.523 | 6.861 | 1.00 | 44.65 | C |
| ATOM | 2098 | C | SER | C | 43 | 7.614 | −2.918 | 8.247 | 1.00 | 44.45 | C |
| ATOM | 2099 | O | SER | C | 43 | 6.772 | −2.999 | 9.140 | 1.00 | 44.56 | O |
| ATOM | 2100 | CB | SER | C | 43 | 6.917 | −2.438 | 5.864 | 1.00 | 44.40 | C |
| ATOM | 2101 | OG | SER | C | 43 | 7.709 | −1.272 | 5.989 | 1.00 | 44.82 | O |
| ATOM | 2102 | N | ASN | C | 44 | 8.800 | −2.326 | 8.416 | 1.00 | 44.11 | N |
| ATOM | 2103 | CA | ASN | C | 44 | 9.137 | −1.528 | 9.602 | 1.00 | 43.78 | C |
| ATOM | 2104 | C | ASN | C | 44 | 8.181 | −0.357 | 9.796 | 1.00 | 43.95 | C |
| ATOM | 2105 | O | ASN | C | 44 | 7.891 | 0.033 | 10.929 | 1.00 | 43.84 | O |
| ATOM | 2106 | CB | ASN | C | 44 | 10.562 | −0.983 | 9.511 | 1.00 | 43.70 | C |
| ATOM | 2107 | CG | ASN | C | 44 | 11.612 | −2.068 | 9.519 | 1.00 | 42.83 | C |
| ATOM | 2108 | OD1 | ASN | C | 44 | 11.300 | −3.250 | 9.561 | 1.00 | 42.52 | O |
| ATOM | 2109 | ND2 | ASN | C | 44 | 12.875 | −1.665 | 9.473 | 1.00 | 42.22 | N |
| ATOM | 2110 | N | VAL | C | 45 | 7.720 | 0.213 | 8.683 | 1.00 | 44.12 | N |
| ATOM | 2111 | CA | VAL | C | 45 | 6.613 | 1.165 | 8.697 | 1.00 | 44.50 | C |
| ATOM | 2112 | C | VAL | C | 45 | 5.425 | 0.501 | 8.008 | 1.00 | 44.74 | C |
| ATOM | 2113 | O | VAL | C | 45 | 5.224 | 0.662 | 6.806 | 1.00 | 44.78 | O |
| ATOM | 2114 | CB | VAL | C | 45 | 6.960 | 2.514 | 7.995 | 1.00 | 44.56 | C |
| ATOM | 2115 | CG1 | VAL | C | 45 | 5.877 | 3.572 | 8.277 | 1.00 | 44.03 | C |
| ATOM | 2116 | CG2 | VAL | C | 45 | 8.331 | 3.028 | 8.426 | 1.00 | 44.52 | C |
| ATOM | 2117 | N | GLN | C | 46 | 4.652 | −0.266 | 8.772 | 1.00 | 45.12 | N |
| ATOM | 2118 | CA | GLN | C | 46 | 3.529 | −1.019 | 8.209 | 1.00 | 45.56 | C |
| ATOM | 2119 | C | GLN | C | 46 | 2.384 | −0.103 | 7.785 | 1.00 | 45.88 | C |
| ATOM | 2120 | O | GLN | C | 46 | 1.838 | 0.655 | 8.588 | 1.00 | 45.68 | O |
| ATOM | 2121 | CB | GLN | C | 46 | 3.041 | −2.108 | 9.178 | 1.00 | 45.54 | C |
| ATOM | 2122 | CG | GLN | C | 46 | 1.803 | −2.877 | 8.714 | 1.00 | 45.50 | C |
| ATOM | 2123 | CD | GLN | C | 46 | 2.064 | −3.814 | 7.541 | 1.00 | 45.56 | C |
| ATOM | 2124 | OE1 | GLN | C | 46 | 3.211 | −4.053 | 7.150 | 1.00 | 45.71 | O |
| ATOM | 2125 | NE2 | GLN | C | 46 | 0.989 | −4.356 | 6.976 | 1.00 | 45.15 | N |
| ATOM | 2126 | N | GLU | C | 47 | 2.048 | −0.178 | 6.501 | 1.00 | 46.31 | N |
| ATOM | 2127 | CA | GLU | C | 47 | 0.951 | 0.584 | 5.937 | 1.00 | 46.79 | C |
| ATOM | 2128 | C | GLU | C | 47 | −0.363 | 0.011 | 6.471 | 1.00 | 46.76 | C |
| ATOM | 2129 | O | GLU | C | 47 | −0.615 | −1.194 | 6.368 | 1.00 | 46.62 | O |
| ATOM | 2130 | CB | GLU | C | 47 | 0.983 | 0.521 | 4.407 | 1.00 | 47.03 | C |
| ATOM | 2131 | CG | GLU | C | 47 | 2.383 | 0.581 | 3.777 | 1.00 | 48.85 | C |
| ATOM | 2132 | CD | GLU | C | 47 | 3.052 | −0.803 | 3.630 | 1.00 | 50.73 | C |
| ATOM | 2133 | OE1 | GLU | C | 47 | 3.003 | −1.623 | 4.585 | 1.00 | 50.45 | O |
| ATOM | 2134 | OE2 | GLU | C | 47 | 3.640 | −1.061 | 2.551 | 1.00 | 50.70 | O |
| ATOM | 2135 | N | VAL | C | 48 | −1.174 | 0.876 | 7.075 | 1.00 | 46.68 | N |
| ATOM | 2136 | CA | VAL | C | 48 | −2.496 | 0.502 | 7.568 | 1.00 | 46.73 | C |
| ATOM | 2137 | C | VAL | C | 48 | −3.508 | 1.462 | 6.954 | 1.00 | 47.19 | C |
| ATOM | 2138 | O | VAL | C | 48 | −3.252 | 2.667 | 6.857 | 1.00 | 47.21 | O |
| ATOM | 2139 | CB | VAL | C | 48 | −2.578 | 0.549 | 9.128 | 1.00 | 46.61 | C |
| ATOM | 2140 | CG1 | VAL | C | 48 | −4.010 | 0.376 | 9.616 | 1.00 | 45.70 | C |
| ATOM | 2141 | CG2 | VAL | C | 48 | −1.688 | −0.510 | 9.747 | 1.00 | 46.25 | C |
| ATOM | 2142 | N | ARG | C | 49 | −4.646 | 0.926 | 6.521 | 1.00 | 47.50 | N |
| ATOM | 2143 | CA | ARG | C | 49 | −5.727 | 1.758 | 6.000 | 1.00 | 48.04 | C |
| ATOM | 2144 | C | ARG | C | 49 | −6.899 | 1.829 | 6.991 | 1.00 | 47.66 | C |
| ATOM | 2145 | O | ARG | C | 49 | −7.300 | 0.825 | 7.583 | 1.00 | 47.88 | O |
| ATOM | 2146 | CB | ARG | C | 49 | −6.167 | 1.296 | 4.600 | 1.00 | 47.90 | C |
| ATOM | 2147 | CG | ARG | C | 49 | −6.226 | −0.219 | 4.426 | 1.00 | 49.19 | C |
| ATOM | 2148 | CD | ARG | C | 49 | −6.576 | −0.631 | 2.996 | 1.00 | 49.56 | C |
| ATOM | 2149 | NE | ARG | C | 49 | −7.016 | −2.032 | 2.933 | 1.00 | 52.98 | N |
| ATOM | 2150 | CZ | ARG | C | 49 | −7.454 | −2.647 | 1.831 | 1.00 | 54.30 | C |
| ATOM | 2151 | NH1 | ARG | C | 49 | −7.524 | −1.999 | 0.670 | 1.00 | 54.13 | N |
| ATOM | 2152 | NH2 | ARG | C | 49 | −7.826 | −3.924 | 1.886 | 1.00 | 54.89 | N |
| ATOM | 2153 | N | CYS | C | 50 | −7.412 | 3.037 | 7.191 | 1.00 | 47.24 | N |
| ATOM | 2154 | CA | CYS | C | 50 | −8.558 | 3.262 | 8.049 | 1.00 | 46.71 | C |
| ATOM | 2155 | C | CYS | C | 50 | −9.825 | 2.708 | 7.394 | 1.00 | 46.39 | C |
| ATOM | 2156 | O | CYS | C | 50 | −9.850 | 2.489 | 6.183 | 1.00 | 46.28 | O |
| ATOM | 2157 | CB | CYS | C | 50 | −8.703 | 4.754 | 8.325 | 1.00 | 46.84 | C |
| ATOM | 2158 | SG | CYS | C | 50 | −9.117 | 5.717 | 6.859 | 1.00 | 47.40 | S |
| ATOM | 2159 | N | PRO | C | 51 | −10.889 | 2.478 | 8.185 | 1.00 | 46.15 | N |
| ATOM | 2160 | CA | PRO | C | 51 | −11.042 | 2.710 | 9.618 | 1.00 | 46.02 | C |
| ATOM | 2161 | C | PRO | C | 51 | −10.325 | 1.678 | 10.482 | 1.00 | 45.97 | C |
| ATOM | 2162 | O | PRO | C | 51 | −10.411 | 0.479 | 10.221 | 1.00 | 46.33 | O |
| ATOM | 2163 | CB | PRO | C | 51 | −12.547 | 2.604 | 9.818 | 1.00 | 45.93 | C |
| ATOM | 2164 | CG | PRO | C | 51 | −12.970 | 1.647 | 8.781 | 1.00 | 46.10 | C |
| ATOM | 2165 | CD | PRO | C | 51 | −12.119 | 1.925 | 7.594 | 1.00 | 46.16 | C |
| ATOM | 2166 | N | VAL | C | 52 | −9.607 | 2.162 | 11.491 | 1.00 | 45.75 | N |
| ATOM | 2167 | CA | VAL | C | 52 | −8.923 | 1.317 | 12.461 | 1.00 | 45.69 | C |
| ATOM | 2168 | C | VAL | C | 52 | −9.164 | 1.903 | 13.839 | 1.00 | 45.49 | C |
| ATOM | 2169 | O | VAL | C | 52 | −9.463 | 3.092 | 13.974 | 1.00 | 45.54 | O |
| ATOM | 2170 | CB | VAL | C | 52 | −7.385 | 1.314 | 12.267 | 1.00 | 45.78 | C |
| ATOM | 2171 | CG1 | VAL | C | 52 | −6.806 | −0.045 | 12.633 | 1.00 | 45.81 | C |
| ATOM | 2172 | CG2 | VAL | C | 52 | −7.003 | 1.690 | 10.854 | 1.00 | 46.38 | C |
| ATOM | 2173 | N | THR | C | 53 | −9.013 | 1.075 | 14.862 | 1.00 | 45.17 | N |
| ATOM | 2174 | CA | THR | C | 53 | −9.008 | 1.561 | 16.223 | 1.00 | 45.15 | C |
| ATOM | 2175 | C | THR | C | 53 | −7.595 | 1.399 | 16.787 | 1.00 | 45.52 | C |
| ATOM | 2176 | O | THR | C | 53 | −7.085 | 0.276 | 16.907 | 1.00 | 45.71 | O |
| ATOM | 2177 | CB | THR | C | 53 | −10.057 | 0.827 | 17.080 | 1.00 | 45.05 | C |
| ATOM | 2178 | OG1 | THR | C | 53 | −11.355 | 1.038 | 16.514 | 1.00 | 44.56 | O |
| ATOM | 2179 | CG2 | THR | C | 53 | −10.042 | 1.331 | 18.523 | 1.00 | 44.82 | C |
| ATOM | 2180 | N | VAL | C | 54 | −6.959 | 2.527 | 17.110 | 1.00 | 45.62 | N |
| ATOM | 2181 | CA | VAL | C | 54 | −5.613 | 2.513 | 17.677 | 1.00 | 45.66 | C |
| ATOM | 2182 | C | VAL | C | 54 | −5.678 | 2.235 | 19.177 | 1.00 | 45.91 | C |
| ATOM | 2183 | O | VAL | C | 54 | −6.451 | 2.871 | 19.903 | 1.00 | 45.73 | O |
| ATOM | 2184 | CB | VAL | C | 54 | −4.854 | 3.833 | 17.425 | 1.00 | 45.71 | C |
| ATOM | 2185 | CG1 | VAL | C | 54 | −3.356 | 3.631 | 17.649 | 1.00 | 45.05 | C |
| ATOM | 2186 | CG2 | VAL | C | 54 | −5.112 | 4.336 | 16.017 | 1.00 | 45.59 | C |
| ATOM | 2187 | N | CYS | C | 55 | −4.854 | 1.281 | 19.617 | 1.00 | 46.13 | N |
| ATOM | 2188 | CA | CYS | C | 55 | −4.782 | 0.846 | 21.007 | 1.00 | 46.20 | C |
| ATOM | 2189 | C | CYS | C | 55 | −3.368 | 0.950 | 21.578 | 1.00 | 46.22 | C |
| ATOM | 2190 | O | CYS | C | 55 | −2.377 | 0.612 | 20.909 | 1.00 | 46.45 | O |
| ATOM | 2191 | CB | CYS | C | 55 | −5.272 | −0.587 | 21.111 | 1.00 | 46.03 | C |
| ATOM | 2192 | SG | CYS | C | 55 | −6.922 | −0.772 | 20.458 | 1.00 | 48.22 | S |
| ATOM | 2193 | N | GLY | C | 56 | −3.280 | 1.413 | 22.820 | 1.00 | 45.93 | N |
| ATOM | 2194 | CA | GLY | C | 56 | −2.001 | 1.545 | 23.495 | 1.00 | 45.61 | C |
| ATOM | 2195 | C | GLY | C | 56 | −1.706 | 0.348 | 24.363 | 1.00 | 45.65 | C |
| ATOM | 2196 | O | GLY | C | 56 | −2.268 | −0.739 | 24.148 | 1.00 | 45.86 | O |
| ATOM | 2197 | N | ASP | C | 57 | −0.835 | 0.553 | 25.352 | 1.00 | 45.27 | N |
| ATOM | 2198 | CA | ASP | C | 57 | −0.350 | −0.513 | 26.230 | 1.00 | 44.94 | C |
| ATOM | 2199 | C | ASP | C | 57 | −1.478 | −1.346 | 26.823 | 1.00 | 44.61 | C |
| ATOM | 2200 | O | ASP | C | 57 | −2.475 | −0.808 | 27.305 | 1.00 | 44.65 | O |
| ATOM | 2201 | CB | ASP | C | 57 | 0.477 | 0.068 | 27.379 | 1.00 | 45.24 | C |
| ATOM | 2202 | CG | ASP | C | 57 | 1.688 | 0.831 | 26.912 | 1.00 | 45.69 | C |
| ATOM | 2203 | OD1 | ASP | C | 57 | 1.544 | 1.748 | 26.098 | 1.00 | 47.68 | O |
| ATOM | 2204 | OD2 | ASP | C | 57 | 2.795 | 0.540 | 27.382 | 1.00 | 47.57 | O |
| ATOM | 2205 | N | VAL | C | 58 | −1.300 | −2.661 | 26.807 | 1.00 | 44.11 | N |
| ATOM | 2206 | CA | VAL | C | 58 | −2.310 | −3.581 | 27.322 | 1.00 | 43.92 | C |
| ATOM | 2207 | C | VAL | C | 58 | −1.797 | −4.349 | 28.554 | 1.00 | 43.89 | C |
| ATOM | 2208 | O | VAL | C | 58 | −2.563 | −4.671 | 29.478 | 1.00 | 43.45 | O |
| ATOM | 2209 | CB | VAL | C | 58 | −2.849 | −4.473 | 26.170 | 1.00 | 43.83 | C |
| ATOM | 2210 | CG1 | VAL | C | 58 | −2.830 | −5.946 | 26.512 | 1.00 | 44.08 | C |
| ATOM | 2211 | CG2 | VAL | C | 58 | −4.226 | −4.000 | 25.756 | 1.00 | 43.56 | C |
| ATOM | 2212 | N | HIS | C | 59 | −0.489 | −4.615 | 28.547 | 1.00 | 43.95 | N |
| ATOM | 2213 | CA | HIS | C | 59 | 0.266 | −5.112 | 29.708 | 1.00 | 43.84 | C |
| ATOM | 2214 | C | HIS | C | 59 | −0.218 | −6.435 | 30.309 | 1.00 | 43.79 | C |
| ATOM | 2215 | O | HIS | C | 59 | −0.245 | −6.601 | 31.526 | 1.00 | 44.24 | O |
| ATOM | 2216 | CB | HIS | C | 59 | 0.375 | −4.024 | 30.788 | 1.00 | 43.56 | C |
| ATOM | 2217 | CG | HIS | C | 59 | 1.289 | −2.895 | 30.422 | 1.00 | 43.83 | C |
| ATOM | 2218 | ND1 | HIS | C | 59 | 2.618 | −3.082 | 30.105 | 1.00 | 44.30 | N |
| ATOM | 2219 | CD2 | HIS | C | 59 | 1.068 | −1.561 | 30.336 | 1.00 | 43.84 | C |
| ATOM | 2220 | CE1 | HIS | C | 59 | 3.175 | −1.915 | 29.834 | 1.00 | 43.54 | C |
| ATOM | 2221 | NE2 | HIS | C | 59 | 2.255 | −0.976 | 29.966 | 1.00 | 43.91 | N |
| ATOM | 2222 | N | GLY | C | 60 | −0.586 | −7.383 | 29.461 | 1.00 | 43.69 | N |
| ATOM | 2223 | CA | GLY | C | 60 | −0.984 | −8.703 | 29.938 | 1.00 | 44.06 | C |
| ATOM | 2224 | C | GLY | C | 60 | −2.221 | −8.748 | 30.823 | 1.00 | 44.21 | C |
| ATOM | 2225 | O | GLY | C | 60 | −2.486 | −9.758 | 31.477 | 1.00 | 44.01 | O |
| ATOM | 2226 | N | GLN | C | 61 | −2.981 | −7.656 | 30.844 | 1.00 | 44.42 | N |
| ATOM | 2227 | CA | GLN | C | 61 | −4.199 | −7.584 | 31.649 | 1.00 | 44.55 | C |
| ATOM | 2228 | C | GLN | C | 61 | −5.383 | −8.128 | 30.862 | 1.00 | 44.64 | C |
| ATOM | 2229 | O | GLN | C | 61 | −6.256 | −7.377 | 30.411 | 1.00 | 44.48 | O |
| ATOM | 2230 | CB | GLN | C | 61 | −4.443 | −6.156 | 32.130 | 1.00 | 44.58 | C |
| ATOM | 2231 | CG | GLN | C | 61 | −3.501 | −5.755 | 33.244 | 1.00 | 44.66 | C |
| ATOM | 2232 | CD | GLN | C | 61 | −3.496 | −4.279 | 33.514 | 1.00 | 45.59 | C |
| ATOM | 2233 | OE1 | GLN | C | 61 | −2.439 | −3.692 | 33.703 | 1.00 | 47.67 | O |
| ATOM | 2234 | NE2 | GLN | C | 61 | −4.671 | −3.661 | 33.532 | 1.00 | 45.90 | N |
| ATOM | 2235 | N | PHE | C | 62 | −5.383 | −9.455 | 30.712 | 1.00 | 44.82 | N |
| ATOM | 2236 | CA | PHE | C | 62 | −6.337 | −10.195 | 29.875 | 1.00 | 44.73 | C |
| ATOM | 2237 | C | PHE | C | 62 | −7.788 | −9.715 | 29.980 | 1.00 | 44.68 | C |
| ATOM | 2238 | O | PHE | C | 62 | −8.431 | −9.449 | 28.961 | 1.00 | 44.79 | O |
| ATOM | 2239 | CB | PHE | C | 62 | −6.243 | −11.701 | 30.161 | 1.00 | 44.59 | C |
| ATOM | 2240 | CG | PHE | C | 62 | −7.339 | −12.502 | 29.529 | 1.00 | 44.70 | C |
| ATOM | 2241 | CD1 | PHE | C | 62 | −7.243 | −12.907 | 28.200 | 1.00 | 44.50 | C |
| ATOM | 2242 | CD2 | PHE | C | 62 | −8.478 | −12.841 | 30.257 | 1.00 | 44.56 | C |
| ATOM | 2243 | CE1 | PHE | C | 62 | −8.264 | −13.644 | 27.604 | 1.00 | 44.06 | C |
| ATOM | 2244 | CE2 | PHE | C | 62 | −9.500 | −13.566 | 29.670 | 1.00 | 44.65 | C |
| ATOM | 2245 | CZ | PHE | C | 62 | −9.391 | −13.974 | 28.338 | 1.00 | 44.67 | C |
| ATOM | 2246 | N | HIS | C | 63 | −8.287 | −9.606 | 31.208 | 1.00 | 44.39 | N |
| ATOM | 2247 | CA | HIS | C | 63 | −9.670 | −9.218 | 31.441 | 1.00 | 44.39 | C |
| ATOM | 2248 | C | HIS | C | 63 | −9.994 | −7.792 | 30.989 | 1.00 | 44.50 | C |
| ATOM | 2249 | O | HIS | C | 63 | −11.079 | −7.538 | 30.462 | 1.00 | 45.03 | O |
| ATOM | 2250 | CB | HIS | C | 63 | −10.033 | −9.454 | 32.902 | 1.00 | 44.22 | C |
| ATOM | 2251 | CG | HIS | C | 63 | −9.858 | −10.880 | 33.321 | 1.00 | 44.22 | C |
| ATOM | 2252 | ND1 | HIS | C | 63 | −8.639 | −11.396 | 33.706 | 1.00 | 43.84 | N |
| ATOM | 2253 | CD2 | HIS | C | 63 | −10.736 | −11.911 | 33.368 | 1.00 | 43.66 | C |
| ATOM | 2254 | CE1 | HIS | C | 63 | −8.779 | −12.677 | 34.000 | 1.00 | 43.36 | C |
| ATOM | 2255 | NE2 | HIS | C | 63 | −10.041 | −13.014 | 33.802 | 1.00 | 43.76 | N |
| ATOM | 2256 | N | ASP | C | 64 | −9.054 | −6.870 | 31.160 | 1.00 | 44.18 | N |
| ATOM | 2257 | CA | ASP | C | 64 | −9.248 | −5.503 | 30.677 | 1.00 | 44.05 | C |
| ATOM | 2258 | C | ASP | C | 64 | −9.187 | −5.410 | 29.154 | 1.00 | 44.21 | C |
| ATOM | 2259 | O | ASP | C | 64 | −9.724 | −4.464 | 28.558 | 1.00 | 44.44 | O |
| ATOM | 2260 | CB | ASP | C | 64 | −8.217 | −4.571 | 31.288 | 1.00 | 43.92 | C |
| ATOM | 2261 | CG | ASP | C | 64 | −8.342 | −4.467 | 32.788 | 1.00 | 43.51 | C |
| ATOM | 2262 | OD1 | ASP | C | 64 | −9.463 | −4.616 | 33.317 | 1.00 | 44.39 | O |
| ATOM | 2263 | OD2 | ASP | C | 64 | −7.312 | −4.217 | 33.443 | 1.00 | 43.31 | O |
| ATOM | 2264 | N | LEU | C | 65 | −8.517 | −6.379 | 28.530 | 1.00 | 44.06 | N |
| ATOM | 2265 | CA | LEU | C | 65 | −8.499 | −6.480 | 27.077 | 1.00 | 43.84 | C |
| ATOM | 2266 | C | LEU | C | 65 | −9.869 | −6.954 | 26.604 | 1.00 | 43.65 | C |
| ATOM | 2267 | O | LEU | C | 65 | −10.371 | −6.496 | 25.579 | 1.00 | 43.79 | O |
| ATOM | 2268 | CB | LEU | C | 65 | −7.393 | −7.429 | 26.608 | 1.00 | 44.06 | C |
| ATOM | 2269 | CG | LEU | C | 65 | −7.380 | −7.900 | 25.146 | 1.00 | 44.19 | C |
| ATOM | 2270 | CD1 | LEU | C | 65 | −6.826 | −6.840 | 24.222 | 1.00 | 44.37 | C |
| ATOM | 2271 | CD2 | LEU | C | 65 | −6.586 | −9.175 | 25.011 | 1.00 | 44.22 | C |
| ATOM | 2272 | N | MET | C | 66 | −10.476 | −7.868 | 27.358 | 1.00 | 43.20 | N |
| ATOM | 2273 | CA | MET | C | 66 | −11.851 | −8.277 | 27.089 | 1.00 | 42.86 | C |
| ATOM | 2274 | C | MET | C | 66 | −12.766 | −7.069 | 27.208 | 1.00 | 42.50 | C |
| ATOM | 2275 | O | MET | C | 66 | −13.661 | −6.867 | 26.383 | 1.00 | 42.61 | O |
| ATOM | 2276 | CB | MET | C | 66 | −12.295 | −9.385 | 28.043 | 1.00 | 42.92 | C |
| ATOM | 2277 | CG | MET | C | 66 | −11.561 | −10.706 | 27.860 | 1.00 | 43.34 | C |
| ATOM | 2278 | SD | MET | C | 66 | −11.833 | −11.463 | 26.243 | 1.00 | 43.58 | S |
| ATOM | 2279 | CE | MET | C | 66 | −10.545 | −10.710 | 25.270 | 1.00 | 43.93 | C |
| ATOM | 2280 | N | GLU | C | 67 | −12.511 | −6.255 | 28.226 | 1.00 | 41.89 | N |
| ATOM | 2281 | CA | GLU | C | 67 | −13.231 | −5.008 | 28.401 | 1.00 | 41.53 | C |
| ATOM | 2282 | C | GLU | C | 67 | −13.032 | −4.095 | 27.187 | 1.00 | 41.45 | C |
| ATOM | 2283 | O | GLU | C | 67 | −13.996 | −3.511 | 26.681 | 1.00 | 41.09 | O |
| ATOM | 2284 | CB | GLU | C | 67 | −12.792 | −4.313 | 29.692 | 1.00 | 41.48 | C |
| ATOM | 2285 | CG | GLU | C | 67 | −13.709 | −3.193 | 30.132 | 1.00 | 40.73 | C |
| ATOM | 2286 | CD | GLU | C | 67 | −15.070 | −3.672 | 30.595 | 1.00 | 40.41 | C |
| ATOM | 2287 | OE1 | GLU | C | 67 | −15.316 | −4.904 | 30.676 | 1.00 | 41.30 | O |
| ATOM | 2288 | OE2 | GLU | C | 67 | −15.901 | −2.797 | 30.895 | 1.00 | 40.10 | O |
| ATOM | 2289 | N | LEU | C | 68 | −11.782 | −3.994 | 26.724 | 1.00 | 41.37 | N |
| ATOM | 2290 | CA | LEU | C | 68 | −11.447 | −3.208 | 25.535 | 1.00 | 41.30 | C |
| ATOM | 2291 | C | LEU | C | 68 | −12.356 | −3.606 | 24.370 | 1.00 | 41.28 | C |
| ATOM | 2292 | O | LEU | C | 68 | −12.890 | −2.741 | 23.674 | 1.00 | 41.29 | O |
| ATOM | 2293 | CB | LEU | C | 68 | −9.966 | −3.382 | 25.166 | 1.00 | 41.17 | C |
| ATOM | 2294 | CG | LEU | C | 68 | −9.301 | −2.535 | 24.066 | 1.00 | 41.22 | C |
| ATOM | 2295 | CD1 | LEU | C | 68 | −7.771 | −2.512 | 24.213 | 1.00 | 41.02 | C |
| ATOM | 2296 | CD2 | LEU | C | 68 | −9.681 | −2.993 | 22.674 | 1.00 | 40.83 | C |
| ATOM | 2297 | N | PHE | C | 69 | −12.548 | −4.912 | 24.187 | 1.00 | 41.24 | N |
| ATOM | 2298 | CA | PHE | C | 69 | −13.346 | −5.427 | 23.081 | 1.00 | 41.27 | C |
| ATOM | 2299 | C | PHE | C | 69 | −14.823 | −5.140 | 23.256 | 1.00 | 41.17 | C |
| ATOM | 2300 | O | PHE | C | 69 | −15.541 | −4.975 | 22.280 | 1.00 | 41.34 | O |
| ATOM | 2301 | CB | PHE | C | 69 | −13.131 | −6.926 | 22.905 | 1.00 | 41.58 | C |
| ATOM | 2302 | CG | PHE | C | 69 | −11.794 | −7.283 | 22.332 | 1.00 | 41.97 | C |
| ATOM | 2303 | CD1 | PHE | C | 69 | −11.346 | −6.695 | 21.153 | 1.00 | 42.79 | C |
| ATOM | 2304 | CD2 | PHE | C | 69 | −10.988 | −8.217 | 22.963 | 1.00 | 41.88 | C |
| ATOM | 2305 | CE1 | PHE | C | 69 | −10.109 | −7.028 | 20.620 | 1.00 | 43.09 | C |
| ATOM | 2306 | CE2 | PHE | C | 69 | −9.757 | −8.563 | 22.437 | 1.00 | 42.27 | C |
| ATOM | 2307 | CZ | PHE | C | 69 | −9.313 | −7.970 | 21.265 | 1.00 | 42.86 | C |
| ATOM | 2308 | N | ARG | C | 70 | −15.279 | −5.093 | 24.501 | 1.00 | 40.95 | N |
| ATOM | 2309 | CA | ARG | C | 70 | −16.653 | −4.718 | 24.778 | 1.00 | 40.54 | C |
| ATOM | 2310 | C | ARG | C | 70 | −16.904 | −3.242 | 24.452 | 1.00 | 40.42 | C |
| ATOM | 2311 | O | ARG | C | 70 | −18.022 | −2.863 | 24.126 | 1.00 | 40.77 | O |
| ATOM | 2312 | CB | ARG | C | 70 | −17.018 | −5.060 | 26.220 | 1.00 | 40.50 | C |
| ATOM | 2313 | CG | ARG | C | 70 | −17.312 | −6.542 | 26.409 | 1.00 | 41.00 | C |
| ATOM | 2314 | CD | ARG | C | 70 | −17.699 | −6.858 | 27.826 | 1.00 | 42.50 | C |
| ATOM | 2315 | NE | ARG | C | 70 | −16.523 | −7.035 | 28.675 | 1.00 | 43.90 | N |
| ATOM | 2316 | CZ | ARG | C | 70 | −16.194 | −8.179 | 29.266 | 1.00 | 43.97 | C |
| ATOM | 2317 | NH1 | ARG | C | 70 | −16.963 | −9.253 | 29.114 | 1.00 | 42.65 | N |
| ATOM | 2318 | NH2 | ARG | C | 70 | −15.104 | −8.243 | 30.023 | 1.00 | 44.31 | N |
| ATOM | 2319 | N | ILE | C | 71 | −15.853 | −2.428 | 24.512 | 1.00 | 40.13 | N |
| ATOM | 2320 | CA | ILE | C | 71 | −15.924 | −1.014 | 24.154 | 1.00 | 39.71 | C |
| ATOM | 2321 | C | ILE | C | 71 | −15.828 | −0.832 | 22.640 | 1.00 | 39.86 | C |
| ATOM | 2322 | O | ILE | C | 71 | −16.795 | −0.419 | 22.005 | 1.00 | 40.06 | O |
| ATOM | 2323 | CB | ILE | C | 71 | −14.815 | −0.180 | 24.867 | 1.00 | 39.65 | C |
| ATOM | 2324 | CG1 | ILE | C | 71 | −14.885 | −0.351 | 26.395 | 1.00 | 39.11 | C |
| ATOM | 2325 | CG2 | ILE | C | 71 | −14.881 | 1.295 | 24.458 | 1.00 | 39.18 | C |
| ATOM | 2326 | CD1 | ILE | C | 71 | −16.152 | 0.177 | 27.059 | 1.00 | 37.63 | C |
| ATOM | 2327 | N | GLY | C | 72 | −14.669 | −1.148 | 22.066 | 1.00 | 39.77 | N |
| ATOM | 2328 | CA | GLY | C | 72 | −14.445 | −0.993 | 20.627 | 1.00 | 39.87 | C |
| ATOM | 2329 | C | GLY | C | 72 | −15.184 | −1.946 | 19.692 | 1.00 | 40.05 | C |
| ATOM | 2330 | O | GLY | C | 72 | −15.411 | −1.612 | 18.525 | 1.00 | 40.25 | O |
| ATOM | 2331 | N | GLY | C | 73 | −15.551 | −3.129 | 20.187 | 1.00 | 39.84 | N |
| ATOM | 2332 | CA | GLY | C | 73 | −16.158 | −4.162 | 19.352 | 1.00 | 39.84 | C |
| ATOM | 2333 | C | GLY | C | 73 | −15.264 | −5.373 | 19.138 | 1.00 | 40.23 | C |
| ATOM | 2334 | O | GLY | C | 73 | −14.055 | −5.306 | 19.350 | 1.00 | 39.85 | O |
| ATOM | 2335 | N | LYS | C | 74 | −15.876 | −6.481 | 18.719 | 1.00 | 40.81 | N |
| ATOM | 2336 | CA | LYS | C | 74 | −15.184 | −7.745 | 18.454 | 1.00 | 41.58 | C |
| ATOM | 2337 | C | LYS | C | 74 | −14.336 | −7.731 | 17.178 | 1.00 | 41.94 | C |
| ATOM | 2338 | O | LYS | C | 74 | −14.686 | −7.100 | 16.176 | 1.00 | 41.96 | O |
| ATOM | 2339 | CB | LYS | C | 74 | −16.193 | −8.891 | 18.341 | 1.00 | 41.64 | C |
| ATOM | 2340 | CG | LYS | C | 74 | −16.566 | −9.581 | 19.647 | 1.00 | 42.75 | C |
| ATOM | 2341 | CD | LYS | C | 74 | −17.881 | −10.345 | 19.481 | 1.00 | 44.07 | C |
| ATOM | 2342 | CE | LYS | C | 74 | −17.956 | −11.580 | 20.375 | 1.00 | 45.73 | C |
| ATOM | 2343 | NZ | LYS | C | 74 | −18.236 | −11.272 | 21.811 | 1.00 | 46.44 | N |
| ATOM | 2344 | N | SER | C | 75 | −13.219 | −8.447 | 17.235 | 1.00 | 42.29 | N |
| ATOM | 2345 | CA | SER | C | 75 | −12.433 | −8.758 | 16.058 | 1.00 | 42.69 | C |
| ATOM | 2346 | C | SER | C | 75 | −13.059 | −9.997 | 15.407 | 1.00 | 42.76 | C |
| ATOM | 2347 | O | SER | C | 75 | −13.484 | −10.914 | 16.114 | 1.00 | 43.02 | O |
| ATOM | 2348 | CB | SER | C | 75 | −10.990 | −9.036 | 16.477 | 1.00 | 42.90 | C |
| ATOM | 2349 | OG | SER | C | 75 | −10.218 | −9.562 | 15.412 | 1.00 | 43.45 | O |
| ATOM | 2350 | N | PRO | C | 76 | −13.114 | −10.047 | 14.062 | 1.00 | 42.69 | N |
| ATOM | 2351 | CA | PRO | C | 76 | −12.564 | −9.161 | 13.037 | 1.00 | 42.62 | C |
| ATOM | 2352 | C | PRO | C | 76 | −13.487 | −8.027 | 12.564 | 1.00 | 42.59 | C |
| ATOM | 2353 | O | PRO | C | 76 | −13.108 | −7.272 | 11.661 | 1.00 | 42.60 | O |
| ATOM | 2354 | CB | PRO | C | 76 | −12.331 | −10.125 | 11.879 | 1.00 | 42.58 | C |
| ATOM | 2355 | CG | PRO | C | 76 | −13.458 | −11.075 | 11.985 | 1.00 | 42.50 | C |
| ATOM | 2356 | CD | PRO | C | 76 | −13.821 | −11.184 | 13.446 | 1.00 | 42.56 | C |
| ATOM | 2357 | N | ASP | C | 77 | −14.681 | −7.919 | 13.146 | 1.00 | 42.44 | N |
| ATOM | 2358 | CA | ASP | C | 77 | −15.627 | −6.871 | 12.767 | 1.00 | 42.23 | C |
| ATOM | 2359 | C | ASP | C | 77 | −14.995 | −5.484 | 12.860 | 1.00 | 42.10 | C |
| ATOM | 2360 | O | ASP | C | 77 | −14.995 | −4.729 | 11.888 | 1.00 | 42.12 | O |
| ATOM | 2361 | CB | ASP | C | 77 | −16.883 | −6.951 | 13.627 | 1.00 | 42.32 | C |
| ATOM | 2362 | CG | ASP | C | 77 | −17.650 | −8.232 | 13.411 | 1.00 | 42.55 | C |
| ATOM | 2363 | OD1 | ASP | C | 77 | −17.595 | −8.782 | 12.286 | 1.00 | 43.05 | O |
| ATOM | 2364 | OD2 | ASP | C | 77 | −18.316 | −8.688 | 14.365 | 1.00 | 42.94 | O |
| ATOM | 2365 | N | THR | C | 78 | −14.445 | −5.170 | 14.029 | 1.00 | 41.90 | N |
| ATOM | 2366 | CA | THR | C | 78 | −13.692 | −3.938 | 14.230 | 1.00 | 41.79 | C |
| ATOM | 2367 | C | THR | C | 78 | −12.221 | −4.181 | 13.878 | 1.00 | 41.98 | C |
| ATOM | 2368 | O | THR | C | 78 | −11.631 | −5.178 | 14.293 | 1.00 | 42.26 | O |
| ATOM | 2369 | CB | THR | C | 78 | −13.810 | −3.433 | 15.702 | 1.00 | 41.65 | C |
| ATOM | 2370 | OG1 | THR | C | 78 | −15.192 | −3.344 | 16.083 | 1.00 | 40.83 | O |
| ATOM | 2371 | CG2 | THR | C | 78 | −13.151 | −2.070 | 15.872 | 1.00 | 41.04 | C |
| ATOM | 2372 | N | ASN | C | 79 | −11.632 | −3.280 | 13.105 | 1.00 | 42.03 | N |
| ATOM | 2373 | CA | ASN | C | 79 | −10.201 | −3.341 | 12.852 | 1.00 | 42.39 | C |
| ATOM | 2374 | C | ASN | C | 79 | −9.438 | −2.691 | 13.991 | 1.00 | 42.99 | C |
| ATOM | 2375 | O | ASN | C | 79 | −9.787 | −1.593 | 14.442 | 1.00 | 43.31 | O |
| ATOM | 2376 | CB | ASN | C | 79 | −9.838 | −2.641 | 11.542 | 1.00 | 42.27 | C |
| ATOM | 2377 | CG | ASN | C | 79 | −10.612 | −3.168 | 10.365 | 1.00 | 41.44 | C |
| ATOM | 2378 | OD1 | ASN | C | 79 | −10.442 | −4.319 | 9.959 | 1.00 | 40.90 | O |
| ATOM | 2379 | ND2 | ASN | C | 79 | −11.467 | −2.324 | 9.800 | 1.00 | 39.82 | N |
| ATOM | 2380 | N | TYR | C | 80 | −8.388 | −3.360 | 14.453 | 1.00 | 43.39 | N |
| ATOM | 2381 | CA | TYR | C | 80 | −7.571 | −2.815 | 15.520 | 1.00 | 43.65 | C |
| ATOM | 2382 | C | TYR | C | 80 | −6.114 | −2.661 | 15.110 | 1.00 | 44.15 | C |
| ATOM | 2383 | O | TYR | C | 80 | −5.592 | −3.410 | 14.276 | 1.00 | 44.09 | O |
| ATOM | 2384 | CB | TYR | C | 80 | −7.643 | −3.705 | 16.759 | 1.00 | 43.55 | C |
| ATOM | 2385 | CG | TYR | C | 80 | −8.934 | −3.638 | 17.541 | 1.00 | 43.24 | C |
| ATOM | 2386 | CD1 | TYR | C | 80 | −9.200 | −2.569 | 18.392 | 1.00 | 43.31 | C |
| ATOM | 2387 | CD2 | TYR | C | 80 | −9.869 | −4.666 | 17.465 | 1.00 | 43.57 | C |
| ATOM | 2388 | CE1 | TYR | C | 80 | −10.374 | −2.515 | 19.135 | 1.00 | 43.32 | C |
| ATOM | 2389 | CE2 | TYR | C | 80 | −11.048 | −4.622 | 18.200 | 1.00 | 43.99 | C |
| ATOM | 2390 | CZ | TYR | C | 80 | −11.293 | −3.545 | 19.036 | 1.00 | 43.47 | C |
| ATOM | 2391 | OH | TYR | C | 80 | −12.457 | −3.498 | 19.765 | 1.00 | 42.82 | O |
| ATOM | 2392 | N | LEU | C | 81 | −5.464 | −1.678 | 15.721 | 1.00 | 44.54 | N |
| ATOM | 2393 | CA | LEU | C | 81 | −4.032 | −1.505 | 15.601 | 1.00 | 44.90 | C |
| ATOM | 2394 | C | LEU | C | 81 | −3.492 | −1.370 | 17.017 | 1.00 | 45.23 | C |
| ATOM | 2395 | O | LEU | C | 81 | −3.829 | −0.424 | 17.739 | 1.00 | 45.61 | O |
| ATOM | 2396 | CB | LEU | C | 81 | −3.701 | −0.282 | 14.721 | 1.00 | 44.98 | C |
| ATOM | 2397 | CG | LEU | C | 81 | −2.382 | 0.495 | 14.810 | 1.00 | 44.81 | C |
| ATOM | 2398 | CD1 | LEU | C | 81 | −1.173 | −0.379 | 14.582 | 1.00 | 45.62 | C |
| ATOM | 2399 | CD2 | LEU | C | 81 | −2.393 | 1.620 | 13.814 | 1.00 | 44.68 | C |
| ATOM | 2400 | N | PHE | C | 82 | −2.678 | −2.341 | 17.416 | 1.00 | 45.27 | N |
| ATOM | 2401 | CA | PHE | C | 82 | −2.095 | −2.356 | 18.752 | 1.00 | 45.29 | C |
| ATOM | 2402 | C | PHE | C | 82 | −0.664 | −1.846 | 18.720 | 1.00 | 45.56 | C |
| ATOM | 2403 | O | PHE | C | 82 | 0.109 | −2.199 | 17.826 | 1.00 | 45.73 | O |
| ATOM | 2404 | CB | PHE | C | 82 | −2.148 | −3.766 | 19.335 | 1.00 | 44.99 | C |
| ATOM | 2405 | CG | PHE | C | 82 | −3.513 | −4.183 | 19.755 | 1.00 | 44.09 | C |
| ATOM | 2406 | CD1 | PHE | C | 82 | −4.361 | −4.822 | 18.863 | 1.00 | 43.30 | C |
| ATOM | 2407 | CD2 | PHE | C | 82 | −3.964 | −3.919 | 21.041 | 1.00 | 44.22 | C |
| ATOM | 2408 | CE1 | PHE | C | 82 | −5.634 | −5.210 | 19.242 | 1.00 | 43.49 | C |
| ATOM | 2409 | CE2 | PHE | C | 82 | −5.244 | −4.300 | 21.437 | 1.00 | 45.15 | C |
| ATOM | 2410 | CZ | PHE | C | 82 | −6.085 | −4.952 | 20.528 | 1.00 | 44.21 | C |
| ATOM | 2411 | N | MET | C | 83 | −0.315 | −1.020 | 19.698 | 1.00 | 45.70 | N |
| ATOM | 2412 | CA | MET | C | 83 | 0.988 | −0.372 | 19.698 | 1.00 | 45.96 | C |
| ATOM | 2413 | C | MET | C | 83 | 1.990 | −1.010 | 20.666 | 1.00 | 45.95 | C |
| ATOM | 2414 | O | MET | C | 83 | 2.933 | −0.356 | 21.091 | 1.00 | 46.34 | O |
| ATOM | 2415 | CB | MET | C | 83 | 0.836 | 1.127 | 19.963 | 1.00 | 45.70 | C |
| ATOM | 2416 | CG | MET | C | 83 | 0.092 | 1.869 | 18.865 | 1.00 | 46.16 | C |
| ATOM | 2417 | SD | MET | C | 83 | 0.094 | 3.673 | 19.001 | 1.00 | 46.99 | S |
| ATOM | 2418 | CE | MET | C | 83 | −0.861 | 3.892 | 20.512 | 1.00 | 46.20 | C |
| ATOM | 2419 | N | GLY | C | 84 | 1.798 | −2.286 | 21.000 | 1.00 | 45.83 | N |
| ATOM | 2420 | CA | GLY | C | 84 | 2.764 | −3.006 | 21.833 | 1.00 | 45.34 | C |
| ATOM | 2421 | C | GLY | C | 84 | 2.432 | −3.136 | 23.313 | 1.00 | 45.24 | C |
| ATOM | 2422 | O | GLY | C | 84 | 1.365 | −2.701 | 23.776 | 1.00 | 45.35 | O |
| ATOM | 2423 | N | ASP | C | 85 | 3.371 | −3.736 | 24.047 | 1.00 | 44.78 | N |
| ATOM | 2424 | CA | ASP | C | 85 | 3.243 | −4.029 | 25.477 | 1.00 | 44.23 | C |
| ATOM | 2425 | C | ASP | C | 85 | 2.012 | −4.883 | 25.777 | 1.00 | 44.22 | C |
| ATOM | 2426 | O | ASP | C | 85 | 1.067 | −4.459 | 26.450 | 1.00 | 44.01 | O |
| ATOM | 2427 | CB | ASP | C | 85 | 3.338 | −2.756 | 26.328 | 1.00 | 44.04 | C |
| ATOM | 2428 | CG | ASP | C | 85 | 4.762 | −2.206 | 26.394 | 1.00 | 44.29 | C |
| ATOM | 2429 | OD1 | ASP | C | 85 | 5.606 | −2.570 | 25.547 | 1.00 | 44.91 | O |
| ATOM | 2430 | OD2 | ASP | C | 85 | 5.059 | −1.406 | 27.298 | 1.00 | 45.30 | O |
| ATOM | 2431 | N | TYR | C | 86 | 2.073 | −6.108 | 25.251 | 1.00 | 44.15 | N |
| ATOM | 2432 | CA | TYR | C | 86 | 1.026 | −7.116 | 25.349 | 1.00 | 44.02 | C |
| ATOM | 2433 | C | TYR | C | 86 | 1.256 | −7.949 | 26.582 | 1.00 | 44.36 | C |
| ATOM | 2434 | O | TYR | C | 86 | 0.348 | −8.634 | 27.076 | 1.00 | 44.54 | O |
| ATOM | 2435 | CB | TYR | C | 86 | 1.060 | −8.016 | 24.112 | 1.00 | 43.63 | C |
| ATOM | 2436 | CG | TYR | C | 86 | 1.105 | −7.224 | 22.834 | 1.00 | 43.45 | C |
| ATOM | 2437 | CD1 | TYR | C | 86 | 0.117 | −6.279 | 22.557 | 1.00 | 44.08 | C |
| ATOM | 2438 | CD2 | TYR | C | 86 | 2.138 | −7.388 | 21.920 | 1.00 | 42.41 | C |
| ATOM | 2439 | CE1 | TYR | C | 86 | 0.144 | −5.532 | 21.404 | 1.00 | 44.53 | C |
| ATOM | 2440 | CE2 | TYR | C | 86 | 2.176 | −6.636 | 20.752 | 1.00 | 43.42 | C |
| ATOM | 2441 | CZ | TYR | C | 86 | 1.169 | −5.708 | 20.500 | 1.00 | 43.76 | C |
| ATOM | 2442 | OH | TYR | C | 86 | 1.166 | −4.935 | 19.362 | 1.00 | 43.04 | O |
| ATOM | 2443 | N | VAL | C | 87 | 2.489 | −7.889 | 27.069 | 1.00 | 44.49 | N |
| ATOM | 2444 | CA | VAL | C | 87 | 2.899 | −8.656 | 28.235 | 1.00 | 44.55 | C |
| ATOM | 2445 | C | VAL | C | 87 | 3.450 | −7.711 | 29.302 | 1.00 | 44.90 | C |
| ATOM | 2446 | O | VAL | C | 87 | 3.983 | −6.640 | 28.976 | 1.00 | 44.78 | O |
| ATOM | 2447 | CB | VAL | C | 87 | 3.930 | −9.772 | 27.858 | 1.00 | 44.47 | C |
| ATOM | 2448 | CG1 | VAL | C | 87 | 3.287 | −10.817 | 26.951 | 1.00 | 43.43 | C |
| ATOM | 2449 | CG2 | VAL | C | 87 | 5.176 | −9.184 | 27.202 | 1.00 | 43.80 | C |
| ATOM | 2450 | N | ASP | C | 88 | 3.271 | −8.093 | 30.567 | 1.00 | 45.10 | N |
| ATOM | 2451 | CA | ASP | C | 88 | 3.845 | −7.371 | 31.704 | 1.00 | 45.51 | C |
| ATOM | 2452 | C | ASP | C | 88 | 4.018 | −8.356 | 32.847 | 1.00 | 45.70 | C |
| ATOM | 2453 | O | ASP | C | 88 | 4.815 | −9.280 | 32.729 | 1.00 | 45.92 | O |
| ATOM | 2454 | CB | ASP | C | 88 | 2.995 | −6.164 | 32.107 | 1.00 | 45.45 | C |
| ATOM | 2455 | CG | ASP | C | 88 | 3.583 | −5.386 | 33.284 | 1.00 | 46.60 | C |
| ATOM | 2456 | OD1 | ASP | C | 88 | 2.805 | −5.006 | 34.185 | 1.00 | 48.82 | O |
| ATOM | 2457 | OD2 | ASP | C | 88 | 4.809 | −5.154 | 33.328 | 1.00 | 46.84 | O |
| ATOM | 2458 | N | ARG | C | 89 | 3.271 | −8.189 | 33.934 | 1.00 | 45.98 | N |
| ATOM | 2459 | CA | ARG | C | 89 | 3.413 | −9.083 | 35.089 | 1.00 | 46.35 | C |
| ATOM | 2460 | C | ARG | C | 89 | 2.910 | −10.507 | 34.775 | 1.00 | 46.44 | C |
| ATOM | 2461 | O | ARG | C | 89 | 2.205 | −10.725 | 33.784 | 1.00 | 46.52 | O |
| ATOM | 2462 | CB | ARG | C | 89 | 2.723 | −8.493 | 36.330 | 1.00 | 46.49 | C |
| ATOM | 2463 | CG | ARG | C | 89 | 3.016 | −7.009 | 36.597 | 1.00 | 47.06 | C |
| ATOM | 2464 | CD | ARG | C | 89 | 4.433 | −6.768 | 37.105 | 1.00 | 48.74 | C |
| ATOM | 2465 | NE | ARG | C | 89 | 5.103 | −5.700 | 36.356 | 1.00 | 50.76 | N |
| ATOM | 2466 | CZ | ARG | C | 89 | 5.353 | −4.472 | 36.817 | 1.00 | 52.13 | C |
| ATOM | 2467 | NH1 | ARG | C | 89 | 4.999 | −4.122 | 38.054 | 1.00 | 51.26 | N |
| ATOM | 2468 | NH2 | ARG | C | 89 | 5.973 | −3.586 | 36.037 | 1.00 | 52.24 | N |
| ATOM | 2469 | N | GLY | C | 90 | 3.285 | −11.473 | 35.612 | 1.00 | 46.55 | N |
| ATOM | 2470 | CA | GLY | C | 90 | 3.006 | −12.883 | 35.339 | 1.00 | 46.41 | C |
| ATOM | 2471 | C | GLY | C | 90 | 1.673 | −13.442 | 35.809 | 1.00 | 46.55 | C |
| ATOM | 2472 | O | GLY | C | 90 | 1.628 | −14.553 | 36.344 | 1.00 | 46.78 | O |
| ATOM | 2473 | N | TYR | C | 91 | 0.583 | −12.702 | 35.608 | 1.00 | 46.55 | N |
| ATOM | 2474 | CA | TYR | C | 91 | −0.747 | −13.200 | 36.002 | 1.00 | 46.60 | C |
| ATOM | 2475 | C | TYR | C | 91 | −1.508 | −13.853 | 34.837 | 1.00 | 46.10 | C |
| ATOM | 2476 | O | TYR | C | 91 | −1.782 | −15.051 | 34.875 | 1.00 | 46.00 | O |
| ATOM | 2477 | CB | TYR | C | 91 | −1.614 | −12.103 | 36.639 | 1.00 | 47.15 | C |
| ATOM | 2478 | CG | TYR | C | 91 | −0.941 | −11.216 | 37.662 | 1.00 | 47.66 | C |
| ATOM | 2479 | CD1 | TYR | C | 91 | −0.520 | −9.935 | 37.313 | 1.00 | 47.96 | C |
| ATOM | 2480 | CD2 | TYR | C | 91 | −0.753 | −11.640 | 38.985 | 1.00 | 48.34 | C |
| ATOM | 2481 | CE1 | TYR | C | 91 | 0.082 | −9.090 | 38.237 | 1.00 | 48.19 | C |
| ATOM | 2482 | CE2 | TYR | C | 91 | −0.145 | −10.799 | 39.930 | 1.00 | 48.80 | C |
| ATOM | 2483 | CZ | TYR | C | 91 | 0.270 | −9.519 | 39.539 | 1.00 | 48.81 | C |
| ATOM | 2484 | OH | TYR | C | 91 | 0.874 | −8.655 | 40.434 | 1.00 | 48.84 | O |
| ATOM | 2485 | N | TYR | C | 92 | −1.845 | −13.056 | 33.818 | 1.00 | 45.51 | N |
| ATOM | 2486 | CA | TYR | C | 92 | −2.588 | −13.523 | 32.643 | 1.00 | 45.04 | C |
| ATOM | 2487 | C | TYR | C | 92 | −1.937 | −13.063 | 31.327 | 1.00 | 44.97 | C |
| ATOM | 2488 | O | TYR | C | 92 | −2.602 | −12.972 | 30.287 | 1.00 | 45.08 | O |
| ATOM | 2489 | CB | TYR | C | 92 | −4.052 | −13.062 | 32.708 | 1.00 | 44.94 | C |
| ATOM | 2490 | CG | TYR | C | 92 | −4.797 | −13.503 | 33.954 | 1.00 | 44.98 | C |
| ATOM | 2491 | CD1 | TYR | C | 92 | −4.922 | −12.648 | 35.053 | 1.00 | 44.51 | C |
| ATOM | 2492 | CD2 | TYR | C | 92 | −5.379 | −14.774 | 34.035 | 1.00 | 44.42 | C |
| ATOM | 2493 | CE1 | TYR | C | 92 | −5.597 | −13.043 | 36.207 | 1.00 | 44.29 | C |
| ATOM | 2494 | CE2 | TYR | C | 92 | −6.060 | −15.183 | 35.186 | 1.00 | 44.57 | C |
| ATOM | 2495 | CZ | TYR | C | 92 | −6.165 | −14.310 | 36.269 | 1.00 | 45.15 | C |
| ATOM | 2496 | OH | TYR | C | 92 | −6.836 | −14.701 | 37.416 | 1.00 | 45.54 | O |
| ATOM | 2497 | N | SER | C | 93 | −0.634 | −12.787 | 31.375 | 1.00 | 44.70 | N |
| ATOM | 2498 | CA | SER | C | 93 | 0.101 | −12.293 | 30.210 | 1.00 | 44.44 | C |
| ATOM | 2499 | C | SER | C | 93 | 0.107 | −13.272 | 29.040 | 1.00 | 44.41 | C |
| ATOM | 2500 | O | SER | C | 93 | 0.067 | −12.856 | 27.882 | 1.00 | 44.29 | O |
| ATOM | 2501 | CB | SER | C | 93 | 1.534 | −11.915 | 30.591 | 1.00 | 44.34 | C |
| ATOM | 2502 | OG | SER | C | 93 | 1.620 | −10.561 | 30.999 | 1.00 | 44.20 | O |
| ATOM | 2503 | N | VAL | C | 94 | 0.154 | −14.568 | 29.351 | 1.00 | 44.39 | N |
| ATOM | 2504 | CA | VAL | C | 94 | 0.166 | −15.621 | 28.328 | 1.00 | 44.17 | C |
| ATOM | 2505 | C | VAL | C | 94 | −1.100 | −15.564 | 27.469 | 1.00 | 44.25 | C |
| ATOM | 2506 | O | VAL | C | 94 | −1.019 | −15.483 | 26.240 | 1.00 | 44.33 | O |
| ATOM | 2507 | CB | VAL | C | 94 | 0.342 | −17.045 | 28.939 | 1.00 | 44.11 | C |
| ATOM | 2508 | CG1 | VAL | C | 94 | 0.279 | −18.110 | 27.857 | 1.00 | 43.33 | C |
| ATOM | 2509 | CG2 | VAL | C | 94 | 1.656 | −17.153 | 29.700 | 1.00 | 43.76 | C |
| ATOM | 2510 | N | GLU | C | 95 | −2.260 | −15.586 | 28.125 | 1.00 | 44.09 | N |
| ATOM | 2511 | CA | GLU | C | 95 | −3.553 | −15.573 | 27.436 | 1.00 | 43.88 | C |
| ATOM | 2512 | C | GLU | C | 95 | −3.702 | −14.313 | 26.584 | 1.00 | 43.61 | C |
| ATOM | 2513 | O | GLU | C | 95 | −4.184 | −14.376 | 25.449 | 1.00 | 43.46 | O |
| ATOM | 2514 | CB | GLU | C | 95 | −4.712 | −15.674 | 28.438 | 1.00 | 43.86 | C |
| ATOM | 2515 | CG | GLU | C | 95 | −4.790 | −16.989 | 29.208 | 1.00 | 44.40 | C |
| ATOM | 2516 | CD | GLU | C | 95 | −3.859 | −17.050 | 30.427 | 1.00 | 45.66 | C |
| ATOM | 2517 | OE1 | GLU | C | 95 | −3.036 | −16.127 | 30.640 | 1.00 | 45.24 | O |
| ATOM | 2518 | OE2 | GLU | C | 95 | −3.958 | −18.043 | 31.182 | 1.00 | 46.14 | O |
| ATOM | 2519 | N | THR | C | 96 | −3.264 | −13.185 | 27.141 | 1.00 | 43.14 | N |
| ATOM | 2520 | CA | THR | C | 96 | −3.351 | −11.892 | 26.482 | 1.00 | 42.88 | C |
| ATOM | 2521 | C | THR | C | 96 | −2.576 | −11.875 | 25.165 | 1.00 | 42.93 | C |
| ATOM | 2522 | O | THR | C | 96 | −3.108 | −11.475 | 24.116 | 1.00 | 43.13 | O |
| ATOM | 2523 | CB | THR | C | 96 | −2.802 | −10.776 | 27.383 | 1.00 | 42.82 | C |
| ATOM | 2524 | OG1 | THR | C | 96 | −3.338 | −10.908 | 28.704 | 1.00 | 42.74 | O |
| ATOM | 2525 | CG2 | THR | C | 96 | −3.169 | −9.432 | 26.832 | 1.00 | 42.56 | C |
| ATOM | 2526 | N | VAL | C | 97 | −1.318 | −12.300 | 25.219 | 1.00 | 42.74 | N |
| ATOM | 2527 | CA | VAL | C | 97 | −0.486 | −12.285 | 24.028 | 1.00 | 42.63 | C |
| ATOM | 2528 | C | VAL | C | 97 | −0.946 | −13.378 | 23.068 | 1.00 | 42.56 | C |
| ATOM | 2529 | O | VAL | C | 97 | −1.142 | −13.111 | 21.880 | 1.00 | 42.93 | O |
| ATOM | 2530 | CB | VAL | C | 97 | 1.036 | −12.364 | 24.356 | 1.00 | 42.74 | C |
| ATOM | 2531 | CG1 | VAL | C | 97 | 1.450 | −13.763 | 24.840 | 1.00 | 42.77 | C |
| ATOM | 2532 | CG2 | VAL | C | 97 | 1.870 | −11.930 | 23.157 | 1.00 | 42.77 | C |
| ATOM | 2533 | N | THR | C | 98 | −1.166 | −14.584 | 23.590 | 1.00 | 41.94 | N |
| ATOM | 2534 | CA | THR | C | 98 | −1.578 | −15.703 | 22.759 | 1.00 | 41.93 | C |
| ATOM | 2535 | C | THR | C | 98 | −2.852 | −15.389 | 21.973 | 1.00 | 41.82 | C |
| ATOM | 2536 | O | THR | C | 98 | −2.969 | −15.774 | 20.810 | 1.00 | 41.92 | O |
| ATOM | 2537 | CB | THR | C | 98 | −1.772 | −16.994 | 23.570 | 1.00 | 42.01 | C |
| ATOM | 2538 | OG1 | THR | C | 98 | −0.662 | −17.177 | 24.453 | 1.00 | 42.45 | O |
| ATOM | 2539 | CG2 | THR | C | 98 | −1.860 | −18.188 | 22.648 | 1.00 | 41.79 | C |
| ATOM | 2540 | N | LEU | C | 99 | −3.791 | −14.685 | 22.604 | 1.00 | 41.54 | N |
| ATOM | 2541 | CA | LEU | C | 99 | −5.024 | −14.281 | 21.931 | 1.00 | 41.13 | C |
| ATOM | 2542 | C | LEU | C | 99 | −4.758 | −13.309 | 20.780 | 1.00 | 41.43 | C |
| ATOM | 2543 | O | LEU | C | 99 | −5.210 | −13.533 | 19.659 | 1.00 | 41.61 | O |
| ATOM | 2544 | CB | LEU | C | 99 | −6.013 | −13.685 | 22.929 | 1.00 | 40.96 | C |
| ATOM | 2545 | CG | LEU | C | 99 | −7.288 | −13.002 | 22.423 | 1.00 | 40.56 | C |
| ATOM | 2546 | CD1 | LEU | C | 99 | −8.062 | −13.847 | 21.407 | 1.00 | 39.27 | C |
| ATOM | 2547 | CD2 | LEU | C | 99 | −8.160 | −12.627 | 23.614 | 1.00 | 40.41 | C |
| ATOM | 2548 | N | LEU | C | 100 | −4.014 | −12.244 | 21.058 | 1.00 | 41.43 | N |
| ATOM | 2549 | CA | LEU | C | 100 | −3.733 | −11.232 | 20.049 | 1.00 | 41.51 | C |
| ATOM | 2550 | C | LEU | C | 100 | −2.947 | −11.788 | 18.854 | 1.00 | 41.60 | C |
| ATOM | 2551 | O | LEU | C | 100 | −3.163 | −11.373 | 17.705 | 1.00 | 41.64 | O |
| ATOM | 2552 | CB | LEU | C | 100 | −3.000 | −10.046 | 20.679 | 1.00 | 41.74 | C |
| ATOM | 2553 | CG | LEU | C | 100 | −3.748 | −9.214 | 21.734 | 1.00 | 41.35 | C |
| ATOM | 2554 | CD1 | LEU | C | 100 | −2.757 | −8.407 | 22.540 | 1.00 | 40.69 | C |
| ATOM | 2555 | CD2 | LEU | C | 100 | −4.765 | −8.294 | 21.094 | 1.00 | 40.26 | C |
| ATOM | 2556 | N | VAL | C | 101 | −2.040 | −12.726 | 19.128 | 1.00 | 41.37 | N |
| ATOM | 2557 | CA | VAL | C | 101 | −1.287 | −13.395 | 18.072 | 1.00 | 41.05 | C |
| ATOM | 2558 | C | VAL | C | 101 | −2.240 | −14.284 | 17.255 | 1.00 | 41.04 | C |
| ATOM | 2559 | O | VAL | C | 101 | −2.181 | −14.302 | 16.019 | 1.00 | 40.76 | O |
| ATOM | 2560 | CB | VAL | C | 101 | −0.105 | −14.237 | 18.644 | 1.00 | 41.07 | C |
| ATOM | 2561 | CG1 | VAL | C | 101 | 0.484 | −15.178 | 17.577 | 1.00 | 41.11 | C |
| ATOM | 2562 | CG2 | VAL | C | 101 | 0.975 | −13.344 | 19.201 | 1.00 | 40.38 | C |
| ATOM | 2563 | N | ALA | C | 102 | −3.124 | −15.000 | 17.954 | 1.00 | 40.87 | N |
| ATOM | 2564 | CA | ALA | C | 102 | −4.074 | −15.901 | 17.302 | 1.00 | 40.87 | C |
| ATOM | 2565 | C | ALA | C | 102 | −4.992 | −15.116 | 16.387 | 1.00 | 40.77 | C |
| ATOM | 2566 | O | ALA | C | 102 | −5.338 | −15.587 | 15.309 | 1.00 | 40.55 | O |
| ATOM | 2567 | CB | ALA | C | 102 | −4.886 | −16.695 | 18.328 | 1.00 | 40.92 | C |
| ATOM | 2568 | N | LEU | C | 103 | −5.370 | −13.914 | 16.819 | 1.00 | 40.77 | N |
| ATOM | 2569 | CA | LEU | C | 103 | −6.203 | −13.038 | 16.007 | 1.00 | 40.74 | C |
| ATOM | 2570 | C | LEU | C | 103 | −5.484 | −12.608 | 14.737 | 1.00 | 40.85 | C |
| ATOM | 2571 | O | LEU | C | 103 | −6.078 | −12.617 | 13.657 | 1.00 | 41.04 | O |
| ATOM | 2572 | CB | LEU | C | 103 | −6.672 | −11.828 | 16.806 | 1.00 | 40.50 | C |
| ATOM | 2573 | CG | LEU | C | 103 | −7.863 | −12.086 | 17.730 | 1.00 | 40.68 | C |
| ATOM | 2574 | CD1 | LEU | C | 103 | −8.320 | −10.784 | 18.402 | 1.00 | 40.09 | C |
| ATOM | 2575 | CD2 | LEU | C | 103 | −9.018 | −12.757 | 16.976 | 1.00 | 40.30 | C |
| ATOM | 2576 | N | LYS | C | 104 | −4.203 | −12.268 | 14.877 | 1.00 | 40.83 | N |
| ATOM | 2577 | CA | LYS | C | 104 | −3.362 | −11.854 | 13.754 | 1.00 | 40.80 | C |
| ATOM | 2578 | C | LYS | C | 104 | −3.189 | −12.958 | 12.722 | 1.00 | 40.69 | C |
| ATOM | 2579 | O | LYS | C | 104 | −3.316 | −12.712 | 11.524 | 1.00 | 40.64 | O |
| ATOM | 2580 | CB | LYS | C | 104 | −1.992 | −11.377 | 14.256 | 1.00 | 41.08 | C |
| ATOM | 2581 | CG | LYS | C | 104 | −1.022 | −10.912 | 13.164 | 1.00 | 41.05 | C |
| ATOM | 2582 | CD | LYS | C | 104 | −1.421 | −9.568 | 12.559 | 1.00 | 40.39 | C |
| ATOM | 2583 | CE | LYS | C | 104 | −0.586 | −9.285 | 11.329 | 1.00 | 40.20 | C |
| ATOM | 2584 | NZ | LYS | C | 104 | −1.052 | −8.088 | 10.580 | 1.00 | 40.38 | N |
| ATOM | 2585 | N | VAL | C | 105 | −2.892 | −14.165 | 13.197 | 1.00 | 40.76 | N |
| ATOM | 2586 | CA | VAL | C | 105 | −2.750 | −15.342 | 12.340 | 1.00 | 40.74 | C |
| ATOM | 2587 | C | VAL | C | 105 | −4.066 | −15.656 | 11.616 | 1.00 | 40.91 | C |
| ATOM | 2588 | O | VAL | C | 105 | −4.067 | −15.950 | 10.418 | 1.00 | 41.10 | O |
| ATOM | 2589 | CB | VAL | C | 105 | −2.249 | −16.564 | 13.144 | 1.00 | 40.56 | C |
| ATOM | 2590 | CG1 | VAL | C | 105 | −2.245 | −17.821 | 12.288 | 1.00 | 41.03 | C |
| ATOM | 2591 | CG2 | VAL | C | 105 | −0.855 | −16.307 | 13.673 | 1.00 | 40.68 | C |
| ATOM | 2592 | N | ARG | C | 106 | −5.178 | −15.570 | 12.343 | 1.00 | 41.08 | N |
| ATOM | 2593 | CA | ARG | C | 106 | −6.504 | −15.785 | 11.773 | 1.00 | 41.26 | C |
| ATOM | 2594 | C | ARG | C | 106 | −6.901 | −14.663 | 10.809 | 1.00 | 41.48 | C |
| ATOM | 2595 | O | ARG | C | 106 | −7.531 | −14.924 | 9.782 | 1.00 | 41.28 | O |
| ATOM | 2596 | CB | ARG | C | 106 | −7.546 | −15.935 | 12.893 | 1.00 | 41.20 | C |
| ATOM | 2597 | CG | ARG | C | 106 | −8.990 | −16.154 | 12.436 | 1.00 | 41.43 | C |
| ATOM | 2598 | CD | ARG | C | 106 | −9.157 | −17.401 | 11.568 | 1.00 | 41.75 | C |
| ATOM | 2599 | NE | ARG | C | 106 | −10.544 | −17.621 | 11.159 | 1.00 | 42.14 | N |
| ATOM | 2600 | CZ | ARG | C | 106 | −11.088 | −17.158 | 10.033 | 1.00 | 43.37 | C |
| ATOM | 2601 | NH1 | ARG | C | 106 | −10.372 | −16.437 | 9.177 | 1.00 | 42.76 | N |
| ATOM | 2602 | NH2 | ARG | C | 106 | −12.361 | −17.424 | 9.756 | 1.00 | 44.14 | N |
| ATOM | 2603 | N | TYR | C | 107 | −6.523 | −13.427 | 11.140 | 1.00 | 41.71 | N |
| ATOM | 2604 | CA | TYR | C | 107 | −6.963 | −12.258 | 10.390 | 1.00 | 42.23 | C |
| ATOM | 2605 | C | TYR | C | 107 | −5.846 | −11.226 | 10.130 | 1.00 | 42.83 | C |
| ATOM | 2606 | O | TYR | C | 107 | −5.760 | −10.202 | 10.822 | 1.00 | 42.63 | O |
| ATOM | 2607 | CB | TYR | C | 107 | −8.113 | −11.572 | 11.130 | 1.00 | 42.22 | C |
| ATOM | 2608 | CG | TYR | C | 107 | −9.334 | −12.412 | 11.453 | 1.00 | 42.19 | C |
| ATOM | 2609 | CD1 | TYR | C | 107 | −10.119 | −12.983 | 10.443 | 1.00 | 42.23 | C |
| ATOM | 2610 | CD2 | TYR | C | 107 | −9.738 | −12.588 | 12.779 | 1.00 | 42.23 | C |
| ATOM | 2611 | CE1 | TYR | C | 107 | −11.265 | −13.738 | 10.755 | 1.00 | 42.45 | C |
| ATOM | 2612 | CE2 | TYR | C | 107 | −10.877 | −13.333 | 13.101 | 1.00 | 42.03 | C |
| ATOM | 2613 | CZ | TYR | C | 107 | −11.636 | −13.905 | 12.092 | 1.00 | 42.32 | C |
| ATOM | 2614 | OH | TYR | C | 107 | −12.757 | −14.635 | 12.429 | 1.00 | 41.69 | O |
| ATOM | 2615 | N | ARG | C | 108 | −5.028 | −11.483 | 9.107 | 1.00 | 43.67 | N |
| ATOM | 2616 | CA | ARG | C | 108 | −3.860 | −10.643 | 8.757 | 1.00 | 44.27 | C |
| ATOM | 2617 | C | ARG | C | 108 | −4.220 | −9.186 | 8.501 | 1.00 | 44.52 | C |
| ATOM | 2618 | O | ARG | C | 108 | −3.538 | −8.276 | 8.959 | 1.00 | 44.64 | O |
| ATOM | 2619 | CB | ARG | C | 108 | −3.176 | −11.166 | 7.485 | 1.00 | 44.40 | C |
| ATOM | 2620 | CG | ARG | C | 108 | −2.864 | −12.646 | 7.455 | 1.00 | 45.36 | C |
| ATOM | 2621 | CD | ARG | C | 108 | −2.098 | −12.987 | 6.190 | 1.00 | 46.65 | C |
| ATOM | 2622 | NE | ARG | C | 108 | −1.826 | −14.420 | 6.084 | 1.00 | 48.39 | N |
| ATOM | 2623 | CZ | ARG | C | 108 | −0.773 | −14.942 | 5.459 | 1.00 | 49.67 | C |
| ATOM | 2624 | NH1 | ARG | C | 108 | 0.129 | −14.147 | 4.888 | 1.00 | 49.87 | N |
| ATOM | 2625 | NH2 | ARG | C | 108 | −0.611 | −16.262 | 5.417 | 1.00 | 50.25 | N |
| ATOM | 2626 | N | GLU | C | 109 | −5.287 | −8.983 | 7.738 | 1.00 | 45.03 | N |
| ATOM | 2627 | CA | GLU | C | 109 | −5.694 | −7.660 | 7.299 | 1.00 | 45.21 | C |
| ATOM | 2628 | C | GLU | C | 109 | −6.540 | −6.952 | 8.336 | 1.00 | 45.26 | C |
| ATOM | 2629 | O | GLU | C | 109 | −6.808 | −5.760 | 8.191 | 1.00 | 45.19 | O |
| ATOM | 2630 | CB | GLU | C | 109 | −6.477 | −7.759 | 5.983 | 1.00 | 45.40 | C |
| ATOM | 2631 | CG | GLU | C | 109 | −5.773 | −8.544 | 4.887 | 1.00 | 46.14 | C |
| ATOM | 2632 | CD | GLU | C | 109 | −4.368 | −8.035 | 4.617 | 1.00 | 47.93 | C |
| ATOM | 2633 | OE1 | GLU | C | 109 | −4.197 | −6.807 | 4.440 | 1.00 | 48.33 | O |
| ATOM | 2634 | OE2 | GLU | C | 109 | −3.433 | −8.866 | 4.583 | 1.00 | 49.02 | O |
| ATOM | 2635 | N | ARG | C | 110 | −6.963 | −7.684 | 9.371 | 1.00 | 45.49 | N |
| ATOM | 2636 | CA | ARG | C | 110 | −7.906 | −7.154 | 10.364 | 1.00 | 45.80 | C |
| ATOM | 2637 | C | ARG | C | 110 | −7.258 | −6.552 | 11.618 | 1.00 | 46.21 | C |
| ATOM | 2638 | O | ARG | C | 110 | −7.867 | −5.706 | 12.282 | 1.00 | 46.43 | O |
| ATOM | 2639 | CB | ARG | C | 110 | −8.958 | −8.197 | 10.745 | 1.00 | 45.63 | C |
| ATOM | 2640 | CG | ARG | C | 110 | −9.838 | −8.656 | 9.588 | 1.00 | 45.51 | C |
| ATOM | 2641 | CD | ARG | C | 110 | −10.725 | −7.542 | 9.038 | 1.00 | 45.40 | C |
| ATOM | 2642 | NE | ARG | C | 110 | −10.743 | −7.576 | 7.578 | 1.00 | 46.26 | N |
| ATOM | 2643 | CZ | ARG | C | 110 | −10.111 | −6.712 | 6.785 | 1.00 | 46.63 | C |
| ATOM | 2644 | NH1 | ARG | C | 110 | −9.421 | −5.701 | 7.292 | 1.00 | 48.32 | N |
| ATOM | 2645 | NH2 | ARG | C | 110 | −10.180 | −6.846 | 5.472 | 1.00 | 46.62 | N |
| ATOM | 2646 | N | ILE | C | 111 | −6.044 | −6.987 | 11.950 | 1.00 | 46.32 | N |
| ATOM | 2647 | CA | ILE | C | 111 | −5.281 | −6.323 | 13.006 | 1.00 | 46.65 | C |
| ATOM | 2648 | C | ILE | C | 111 | −3.807 | −6.123 | 12.697 | 1.00 | 46.41 | C |
| ATOM | 2649 | O | ILE | C | 111 | −3.205 | −6.868 | 11.916 | 1.00 | 46.29 | O |
| ATOM | 2650 | CB | ILE | C | 111 | −5.374 | −7.017 | 14.396 | 1.00 | 46.97 | C |
| ATOM | 2651 | CG1 | ILE | C | 111 | −5.004 | −8.497 | 14.320 | 1.00 | 47.60 | C |
| ATOM | 2652 | CG2 | ILE | C | 111 | −6.727 | −6.776 | 15.041 | 1.00 | 47.63 | C |
| ATOM | 2653 | CD1 | ILE | C | 111 | −4.440 | −8.987 | 15.633 | 1.00 | 49.99 | C |
| ATOM | 2654 | N | THR | C | 112 | −3.245 | −5.109 | 13.347 | 1.00 | 46.00 | N |
| ATOM | 2655 | CA | THR | C | 112 | −1.826 | −4.839 | 13.304 | 1.00 | 45.63 | C |
| ATOM | 2656 | C | THR | C | 112 | −1.286 | −4.779 | 14.734 | 1.00 | 45.43 | C |
| ATOM | 2657 | O | THR | C | 112 | −1.782 | −4.022 | 15.579 | 1.00 | 45.11 | O |
| ATOM | 2658 | CB | THR | C | 112 | −1.542 | −3.522 | 12.559 | 1.00 | 45.84 | C |
| ATOM | 2659 | OG1 | THR | C | 112 | −2.150 | −3.568 | 11.259 | 1.00 | 45.54 | O |
| ATOM | 2660 | CG2 | THR | C | 112 | −0.039 | −3.284 | 12.418 | 1.00 | 45.45 | C |
| ATOM | 2661 | N | ILE | C | 113 | −0.271 | −5.592 | 14.998 | 1.00 | 45.18 | N |
| ATOM | 2662 | CA | ILE | C | 113 | 0.366 | −5.609 | 16.310 | 1.00 | 45.13 | C |
| ATOM | 2663 | C | ILE | C | 113 | 1.834 | −5.185 | 16.229 | 1.00 | 44.98 | C |
| ATOM | 2664 | O | ILE | C | 113 | 2.676 | −5.922 | 15.718 | 1.00 | 45.19 | O |
| ATOM | 2665 | CB | ILE | C | 113 | 0.203 | −6.990 | 17.031 | 1.00 | 45.15 | C |
| ATOM | 2666 | CG1 | ILE | C | 113 | 0.539 | −8.150 | 16.090 | 1.00 | 44.88 | C |
| ATOM | 2667 | CG2 | ILE | C | 113 | −1.218 | −7.136 | 17.584 | 1.00 | 45.17 | C |
| ATOM | 2668 | CD1 | ILE | C | 113 | 0.723 | −9.472 | 16.784 | 1.00 | 44.97 | C |
| ATOM | 2669 | N | LEU | C | 114 | 2.126 | −3.983 | 16.721 | 1.00 | 44.69 | N |
| ATOM | 2670 | CA | LEU | C | 114 | 3.492 | −3.458 | 16.723 | 1.00 | 44.46 | C |
| ATOM | 2671 | C | LEU | C | 114 | 4.307 | −4.030 | 17.883 | 1.00 | 44.49 | C |
| ATOM | 2672 | O | LEU | C | 114 | 3.781 | −4.721 | 18.750 | 1.00 | 44.49 | O |
| ATOM | 2673 | CB | LEU | C | 114 | 3.492 | −1.919 | 16.777 | 1.00 | 44.31 | C |
| ATOM | 2674 | CG | LEU | C | 114 | 2.723 | −1.106 | 15.724 | 1.00 | 44.18 | C |
| ATOM | 2675 | CD1 | LEU | C | 114 | 3.021 | 0.385 | 15.833 | 1.00 | 43.04 | C |
| ATOM | 2676 | CD2 | LEU | C | 114 | 3.016 | −1.602 | 14.313 | 1.00 | 44.11 | C |
| ATOM | 2677 | N | ARG | C | 115 | 5.601 | −3.743 | 17.890 | 1.00 | 44.57 | N |
| ATOM | 2678 | CA | ARG | C | 115 | 6.453 | −4.139 | 18.992 | 1.00 | 44.45 | C |
| ATOM | 2679 | C | ARG | C | 115 | 6.545 | −3.037 | 20.037 | 1.00 | 44.35 | C |
| ATOM | 2680 | O | ARG | C | 115 | 6.660 | −1.857 | 19.700 | 1.00 | 44.23 | O |
| ATOM | 2681 | CB | ARG | C | 115 | 7.842 | −4.467 | 18.473 | 1.00 | 44.59 | C |
| ATOM | 2682 | CG | ARG | C | 115 | 8.675 | −5.289 | 19.427 | 1.00 | 45.13 | C |
| ATOM | 2683 | CD | ARG | C | 115 | 9.962 | −5.675 | 18.771 | 1.00 | 46.00 | C |
| ATOM | 2684 | NE | ARG | C | 115 | 10.706 | −6.639 | 19.568 | 1.00 | 48.05 | N |
| ATOM | 2685 | CZ | ARG | C | 115 | 11.849 | −7.201 | 19.183 | 1.00 | 48.48 | C |
| ATOM | 2686 | NH1 | ARG | C | 115 | 12.375 | −6.899 | 17.999 | 1.00 | 48.50 | N |
| ATOM | 2687 | NH2 | ARG | C | 115 | 12.461 | −8.070 | 19.978 | 1.00 | 47.81 | N |
| ATOM | 2688 | N | GLY | C | 116 | 6.490 | −3.432 | 21.307 | 1.00 | 44.29 | N |
| ATOM | 2689 | CA | GLY | C | 116 | 6.726 | −2.515 | 22.423 | 1.00 | 43.64 | C |
| ATOM | 2690 | C | GLY | C | 116 | 8.031 | −2.867 | 23.106 | 1.00 | 43.46 | C |
| ATOM | 2691 | O | GLY | C | 116 | 8.631 | −3.900 | 22.829 | 1.00 | 43.34 | O |
| ATOM | 2692 | N | ASN | C | 117 | 8.471 | −2.006 | 24.009 | 1.00 | 43.51 | N |
| ATOM | 2693 | CA | ASN | C | 117 | 9.656 | −2.264 | 24.815 | 1.00 | 43.51 | C |
| ATOM | 2694 | C | ASN | C | 117 | 9.538 | −3.525 | 25.693 | 1.00 | 43.60 | C |
| ATOM | 2695 | O | ASN | C | 117 | 10.548 | −4.076 | 26.142 | 1.00 | 43.42 | O |
| ATOM | 2696 | CB | ASN | C | 117 | 9.994 | −1.027 | 25.659 | 1.00 | 43.31 | C |
| ATOM | 2697 | CG | ASN | C | 117 | 8.991 | −0.781 | 26.788 | 1.00 | 43.61 | C |
| ATOM | 2698 | OD1 | ASN | C | 117 | 9.388 | −0.532 | 27.930 | 1.00 | 43.80 | O |
| ATOM | 2699 | ND2 | ASN | C | 117 | 7.694 | −0.857 | 26.480 | 1.00 | 42.46 | N |
| ATOM | 2700 | N | HIS | C | 118 | 8.307 | −3.975 | 25.929 | 1.00 | 43.89 | N |
| ATOM | 2701 | CA | HIS | C | 118 | 8.057 | −5.187 | 26.719 | 1.00 | 44.30 | C |
| ATOM | 2702 | C | HIS | C | 118 | 8.244 | −6.472 | 25.931 | 1.00 | 44.61 | C |
| ATOM | 2703 | O | HIS | C | 118 | 8.551 | −7.515 | 26.502 | 1.00 | 44.61 | O |
| ATOM | 2704 | CB | HIS | C | 118 | 6.655 | −5.153 | 27.313 | 1.00 | 44.25 | C |
| ATOM | 2705 | CG | HIS | C | 118 | 6.561 | −4.364 | 28.579 | 1.00 | 44.20 | C |
| ATOM | 2706 | ND1 | HIS | C | 118 | 5.774 | −4.753 | 29.640 | 1.00 | 43.76 | N |
| ATOM | 2707 | CD2 | HIS | C | 118 | 7.172 | −3.218 | 28.962 | 1.00 | 44.16 | C |
| ATOM | 2708 | CE1 | HIS | C | 118 | 5.893 | −3.875 | 30.618 | 1.00 | 44.29 | C |
| ATOM | 2709 | NE2 | HIS | C | 118 | 6.734 | −2.933 | 30.232 | 1.00 | 45.10 | N |
| ATOM | 2710 | N | GLU | C | 119 | 8.057 | −6.395 | 24.621 | 1.00 | 45.18 | N |
| ATOM | 2711 | CA | GLU | C | 119 | 8.225 | −7.547 | 23.756 | 1.00 | 46.00 | C |
| ATOM | 2712 | C | GLU | C | 119 | 9.706 | −7.821 | 23.474 | 1.00 | 46.42 | C |
| ATOM | 2713 | O | GLU | C | 119 | 10.167 | −7.759 | 22.319 | 1.00 | 46.34 | O |
| ATOM | 2714 | CB | GLU | C | 119 | 7.439 | −7.362 | 22.461 | 1.00 | 46.30 | C |
| ATOM | 2715 | CG | GLU | C | 119 | 5.952 | −7.634 | 22.603 | 1.00 | 47.33 | C |
| ATOM | 2716 | CD | GLU | C | 119 | 5.231 | −6.526 | 23.322 | 1.00 | 48.72 | C |
| ATOM | 2717 | OE1 | GLU | C | 119 | 5.145 | −5.412 | 22.762 | 1.00 | 48.39 | O |
| ATOM | 2718 | OE2 | GLU | C | 119 | 4.757 | −6.775 | 24.453 | 1.00 | 50.38 | O |
| ATOM | 2719 | N | SER | C | 120 | 10.431 | −8.141 | 24.548 | 1.00 | 46.64 | N |
| ATOM | 2720 | CA | SER | C | 120 | 11.871 | −8.376 | 24.508 | 1.00 | 46.84 | C |
| ATOM | 2721 | C | SER | C | 120 | 12.274 | −9.447 | 25.509 | 1.00 | 46.80 | C |
| ATOM | 2722 | O | SER | C | 120 | 11.641 | −9.601 | 26.549 | 1.00 | 46.96 | O |
| ATOM | 2723 | CB | SER | C | 120 | 12.621 | −7.084 | 24.836 | 1.00 | 46.94 | C |
| ATOM | 2724 | OG | SER | C | 120 | 12.170 | −6.536 | 26.069 | 1.00 | 47.84 | O |
| ATOM | 2725 | N | ARG | C | 121 | 13.340 | −10.173 | 25.193 | 1.00 | 46.78 | N |
| ATOM | 2726 | CA | ARG | C | 121 | 13.911 | −11.151 | 26.103 | 1.00 | 46.69 | C |
| ATOM | 2727 | C | ARG | C | 121 | 14.304 | −10.488 | 27.423 | 1.00 | 46.92 | C |
| ATOM | 2728 | O | ARG | C | 121 | 14.143 | −11.081 | 28.493 | 1.00 | 46.75 | O |
| ATOM | 2729 | CB | ARG | C | 121 | 15.127 | −11.820 | 25.458 | 1.00 | 46.58 | C |
| ATOM | 2730 | CG | ARG | C | 121 | 14.773 | −12.794 | 24.352 | 1.00 | 46.38 | C |
| ATOM | 2731 | CD | ARG | C | 121 | 15.934 | −13.030 | 23.397 | 1.00 | 46.31 | C |
| ATOM | 2732 | NE | ARG | C | 121 | 15.480 | −13.628 | 22.142 | 1.00 | 46.13 | N |
| ATOM | 2733 | CZ | ARG | C | 121 | 15.010 | −12.941 | 21.099 | 1.00 | 46.38 | C |
| ATOM | 2734 | NH1 | ARG | C | 121 | 14.928 | −11.615 | 21.139 | 1.00 | 45.86 | N |
| ATOM | 2735 | NH2 | ARG | C | 121 | 14.611 | −13.587 | 20.011 | 1.00 | 46.30 | N |
| ATOM | 2736 | N | GLN | C | 122 | 14.798 | −9.253 | 27.332 | 1.00 | 47.18 | N |
| ATOM | 2737 | CA | GLN | C | 122 | 15.299 | −8.516 | 28.486 | 1.00 | 47.73 | C |
| ATOM | 2738 | C | GLN | C | 122 | 14.226 | −8.322 | 29.565 | 1.00 | 47.76 | C |
| ATOM | 2739 | O | GLN | C | 122 | 14.336 | −8.861 | 30.668 | 1.00 | 47.93 | O |
| ATOM | 2740 | CB | GLN | C | 122 | 15.858 | −7.164 | 28.043 | 1.00 | 47.98 | C |
| ATOM | 2741 | CG | GLN | C | 122 | 16.846 | −6.533 | 29.036 | 1.00 | 49.38 | C |
| ATOM | 2742 | CD | GLN | C | 122 | 16.758 | −5.001 | 29.108 | 1.00 | 50.99 | C |
| ATOM | 2743 | OE1 | GLN | C | 122 | 16.196 | −4.338 | 28.223 | 1.00 | 51.61 | O |
| ATOM | 2744 | NE2 | GLN | C | 122 | 17.318 | −4.437 | 30.175 | 1.00 | 51.04 | N |
| ATOM | 2745 | N | ILE | C | 123 | 13.181 | −7.571 | 29.232 | 1.00 | 47.76 | N |
| ATOM | 2746 | CA | ILE | C | 123 | 12.125 | −7.245 | 30.187 | 1.00 | 47.66 | C |
| ATOM | 2747 | C | ILE | C | 123 | 11.259 | −8.445 | 30.605 | 1.00 | 47.64 | C |
| ATOM | 2748 | O | ILE | C | 123 | 10.976 | −8.619 | 31.798 | 1.00 | 47.74 | O |
| ATOM | 2749 | CB | ILE | C | 123 | 11.280 | −6.043 | 29.691 | 1.00 | 47.66 | C |
| ATOM | 2750 | CG1 | ILE | C | 123 | 12.055 | −4.741 | 29.927 | 1.00 | 47.56 | C |
| ATOM | 2751 | CG2 | ILE | C | 123 | 9.922 | −5.991 | 30.379 | 1.00 | 47.52 | C |
| ATOM | 2752 | CD1 | ILE | C | 123 | 11.310 | −3.479 | 29.526 | 1.00 | 47.97 | C |
| ATOM | 2753 | N | THR | C | 124 | 10.870 | −9.282 | 29.640 | 1.00 | 47.35 | N |
| ATOM | 2754 | CA | THR | C | 124 | 10.057 | −10.481 | 29.926 | 1.00 | 47.05 | C |
| ATOM | 2755 | C | THR | C | 124 | 10.674 | −11.448 | 30.933 | 1.00 | 46.85 | C |
| ATOM | 2756 | O | THR | C | 124 | 9.950 | −12.075 | 31.703 | 1.00 | 46.55 | O |
| ATOM | 2757 | CB | THR | C | 124 | 9.733 | −11.295 | 28.661 | 1.00 | 47.05 | C |
| ATOM | 2758 | OG1 | THR | C | 124 | 10.951 | −11.618 | 27.971 | 1.00 | 46.44 | O |
| ATOM | 2759 | CG2 | THR | C | 124 | 8.766 | −10.530 | 27.747 | 1.00 | 46.87 | C |
| ATOM | 2760 | N | GLN | C | 125 | 12.000 | −11.580 | 30.914 | 1.00 | 46.90 | N |
| ATOM | 2761 | CA | GLN | C | 125 | 12.687 | −12.490 | 31.836 | 1.00 | 47.27 | C |
| ATOM | 2762 | C | GLN | C | 125 | 12.545 | −12.011 | 33.265 | 1.00 | 47.12 | C |
| ATOM | 2763 | O | GLN | C | 125 | 12.456 | −12.805 | 34.194 | 1.00 | 46.80 | O |
| ATOM | 2764 | CB | GLN | C | 125 | 14.172 | −12.640 | 31.480 | 1.00 | 47.16 | C |
| ATOM | 2765 | CG | GLN | C | 125 | 14.518 | −13.978 | 30.837 | 1.00 | 47.39 | C |
| ATOM | 2766 | CD | GLN | C | 125 | 15.904 | −14.013 | 30.183 | 1.00 | 47.88 | C |
| ATOM | 2767 | OE1 | GLN | C | 125 | 16.490 | −15.091 | 30.013 | 1.00 | 48.62 | O |
| ATOM | 2768 | NE2 | GLN | C | 125 | 16.427 | −12.840 | 29.806 | 1.00 | 47.72 | N |
| ATOM | 2769 | N | VAL | C | 126 | 12.470 | −10.695 | 33.406 | 1.00 | 47.38 | N |
| ATOM | 2770 | CA | VAL | C | 126 | 12.616 | −10.019 | 34.682 | 1.00 | 47.47 | C |
| ATOM | 2771 | C | VAL | C | 126 | 11.291 | −9.495 | 35.289 | 1.00 | 47.51 | C |
| ATOM | 2772 | O | VAL | C | 126 | 11.177 | −9.394 | 36.512 | 1.00 | 47.61 | O |
| ATOM | 2773 | CB | VAL | C | 126 | 13.721 | −8.919 | 34.536 | 1.00 | 47.53 | C |
| ATOM | 2774 | CG1 | VAL | C | 126 | 13.239 | −7.527 | 34.932 | 1.00 | 47.78 | C |
| ATOM | 2775 | CG2 | VAL | C | 126 | 14.991 | −9.330 | 35.262 | 1.00 | 47.38 | C |
| ATOM | 2776 | N | TYR | C | 127 | 10.297 | −9.192 | 34.446 | 1.00 | 47.39 | N |
| ATOM | 2777 | CA | TYR | C | 127 | 9.026 | −8.595 | 34.911 | 1.00 | 47.28 | C |
| ATOM | 2778 | C | TYR | C | 127 | 7.960 | −9.603 | 35.321 | 1.00 | 47.09 | C |
| ATOM | 2779 | O | TYR | C | 127 | 7.166 | −9.324 | 36.222 | 1.00 | 47.17 | O |
| ATOM | 2780 | CB | TYR | C | 127 | 8.437 | −7.622 | 33.875 | 1.00 | 47.27 | C |
| ATOM | 2781 | CG | TYR | C | 127 | 8.987 | −6.207 | 33.935 | 1.00 | 47.55 | C |
| ATOM | 2782 | CD1 | TYR | C | 127 | 10.209 | −5.930 | 34.552 | 1.00 | 47.60 | C |
| ATOM | 2783 | CD2 | TYR | C | 127 | 8.295 | −5.145 | 33.345 | 1.00 | 48.08 | C |
| ATOM | 2784 | CE1 | TYR | C | 127 | 10.720 | −4.639 | 34.593 | 1.00 | 47.69 | C |
| ATOM | 2785 | CE2 | TYR | C | 127 | 8.802 | −3.842 | 33.380 | 1.00 | 47.91 | C |
| ATOM | 2786 | CZ | TYR | C | 127 | 10.017 | −3.601 | 34.006 | 1.00 | 47.73 | C |
| ATOM | 2787 | OH | TYR | C | 127 | 10.538 | −2.329 | 34.047 | 1.00 | 47.38 | O |
| ATOM | 2788 | N | GLY | C | 128 | 7.923 | −10.756 | 34.655 | 1.00 | 46.88 | N |
| ATOM | 2789 | CA | GLY | C | 128 | 6.976 | −11.803 | 35.030 | 1.00 | 46.62 | C |
| ATOM | 2790 | C | GLY | C | 128 | 6.644 | −12.843 | 33.981 | 1.00 | 46.57 | C |
| ATOM | 2791 | O | GLY | C | 128 | 6.384 | −14.003 | 34.323 | 1.00 | 46.36 | O |
| ATOM | 2792 | N | PHE | C | 129 | 6.655 | −12.435 | 32.709 | 1.00 | 46.47 | N |
| ATOM | 2793 | CA | PHE | C | 129 | 6.189 | −13.289 | 31.610 | 1.00 | 46.45 | C |
| ATOM | 2794 | C | PHE | C | 129 | 6.876 | −14.653 | 31.555 | 1.00 | 46.49 | C |
| ATOM | 2795 | O | PHE | C | 129 | 6.224 | −15.670 | 31.309 | 1.00 | 46.50 | O |
| ATOM | 2796 | CB | PHE | C | 129 | 6.301 | −12.576 | 30.260 | 1.00 | 46.30 | C |
| ATOM | 2797 | CG | PHE | C | 129 | 5.666 | −13.330 | 29.115 | 1.00 | 46.66 | C |
| ATOM | 2798 | CD1 | PHE | C | 129 | 4.372 | −13.847 | 29.227 | 1.00 | 46.87 | C |
| ATOM | 2799 | CD2 | PHE | C | 129 | 6.355 | −13.512 | 27.918 | 1.00 | 46.87 | C |
| ATOM | 2800 | CE1 | PHE | C | 129 | 3.780 | −14.544 | 28.169 | 1.00 | 46.93 | C |
| ATOM | 2801 | CE2 | PHE | C | 129 | 5.769 | −14.210 | 26.847 | 1.00 | 46.76 | C |
| ATOM | 2802 | CZ | PHE | C | 129 | 4.482 | −14.725 | 26.974 | 1.00 | 46.52 | C |
| ATOM | 2803 | N | TYR | C | 130 | 8.185 | −14.661 | 31.796 | 1.00 | 46.55 | N |
| ATOM | 2804 | CA | TYR | C | 130 | 8.974 | −15.884 | 31.806 | 1.00 | 46.61 | C |
| ATOM | 2805 | C | TYR | C | 130 | 8.555 | −16.804 | 32.951 | 1.00 | 46.75 | C |
| ATOM | 2806 | O | TYR | C | 130 | 8.487 | −18.016 | 32.771 | 1.00 | 46.81 | O |
| ATOM | 2807 | CB | TYR | C | 130 | 10.462 | −15.552 | 31.885 | 1.00 | 46.52 | C |
| ATOM | 2808 | CG | TYR | C | 130 | 11.374 | −16.755 | 31.965 | 1.00 | 46.76 | C |
| ATOM | 2809 | CD1 | TYR | C | 130 | 11.832 | −17.385 | 30.807 | 1.00 | 47.08 | C |
| ATOM | 2810 | CD2 | TYR | C | 130 | 11.797 | −17.252 | 33.196 | 1.00 | 46.80 | C |
| ATOM | 2811 | CE1 | TYR | C | 130 | 12.681 | −18.485 | 30.871 | 1.00 | 46.73 | C |
| ATOM | 2812 | CE2 | TYR | C | 130 | 12.644 | −18.354 | 33.273 | 1.00 | 46.95 | C |
| ATOM | 2813 | CZ | TYR | C | 130 | 13.081 | −18.967 | 32.108 | 1.00 | 46.95 | C |
| ATOM | 2814 | OH | TYR | C | 130 | 13.922 | −20.055 | 32.182 | 1.00 | 46.89 | O |
| ATOM | 2815 | N | ASP | C | 131 | 8.272 | −16.224 | 34.117 | 1.00 | 46.99 | N |
| ATOM | 2816 | CA | ASP | C | 131 | 7.761 | −16.975 | 35.271 | 1.00 | 47.14 | C |
| ATOM | 2817 | C | ASP | C | 131 | 6.393 | −17.606 | 35.002 | 1.00 | 46.98 | C |
| ATOM | 2818 | O | ASP | C | 131 | 6.146 | −18.750 | 35.375 | 1.00 | 46.82 | O |
| ATOM | 2819 | CB | ASP | C | 131 | 7.684 | −16.078 | 36.508 | 1.00 | 47.36 | C |
| ATOM | 2820 | CG | ASP | C | 131 | 8.994 | −16.018 | 37.263 | 1.00 | 48.16 | C |
| ATOM | 2821 | OD1 | ASP | C | 131 | 9.385 | −17.043 | 37.856 | 1.00 | 49.39 | O |
| ATOM | 2822 | OD2 | ASP | C | 131 | 9.631 | −14.945 | 37.277 | 1.00 | 49.49 | O |
| ATOM | 2823 | N | GLU | C | 132 | 5.514 | −16.850 | 34.349 | 1.00 | 46.93 | N |
| ATOM | 2824 | CA | GLU | C | 132 | 4.180 | −17.327 | 34.012 | 1.00 | 46.81 | C |
| ATOM | 2825 | C | GLU | C | 132 | 4.247 | −18.550 | 33.097 | 1.00 | 46.72 | C |
| ATOM | 2826 | O | GLU | C | 132 | 3.604 | −19.570 | 33.368 | 1.00 | 46.65 | O |
| ATOM | 2827 | CB | GLU | C | 132 | 3.375 | −16.210 | 33.356 | 1.00 | 46.80 | C |
| ATOM | 2828 | CG | GLU | C | 132 | 1.874 | −16.393 | 33.464 | 1.00 | 47.11 | C |
| ATOM | 2829 | CD | GLU | C | 132 | 1.101 | −15.363 | 32.670 | 1.00 | 47.95 | C |
| ATOM | 2830 | OE1 | GLU | C | 132 | 1.628 | −14.251 | 32.443 | 1.00 | 48.38 | O |
| ATOM | 2831 | OE2 | GLU | C | 132 | −0.039 | −15.669 | 32.269 | 1.00 | 48.92 | O |
| ATOM | 2832 | N | CYS | C | 133 | 5.039 | −18.436 | 32.029 | 1.00 | 46.56 | N |
| ATOM | 2833 | CA | CYS | C | 133 | 5.323 | −19.544 | 31.109 | 1.00 | 46.49 | C |
| ATOM | 2834 | C | CYS | C | 133 | 5.822 | −20.796 | 31.843 | 1.00 | 46.42 | C |
| ATOM | 2835 | O | CYS | C | 133 | 5.538 | −21.924 | 31.435 | 1.00 | 46.45 | O |
| ATOM | 2836 | CB | CYS | C | 133 | 6.364 | −19.121 | 30.065 | 1.00 | 46.41 | C |
| ATOM | 2837 | SG | CYS | C | 133 | 5.850 | −17.810 | 28.937 | 1.00 | 46.23 | S |
| ATOM | 2838 | N | LEU | C | 134 | 6.564 | −20.581 | 32.927 | 1.00 | 46.25 | N |
| ATOM | 2839 | CA | LEU | C | 134 | 7.148 | −21.665 | 33.706 | 1.00 | 46.05 | C |
| ATOM | 2840 | C | LEU | C | 134 | 6.081 | −22.370 | 34.520 | 1.00 | 45.78 | C |
| ATOM | 2841 | O | LEU | C | 134 | 6.032 | −23.599 | 34.556 | 1.00 | 45.82 | O |
| ATOM | 2842 | CB | LEU | C | 134 | 8.221 | −21.120 | 34.646 | 1.00 | 46.07 | C |
| ATOM | 2843 | CG | LEU | C | 134 | 9.525 | −21.901 | 34.708 | 1.00 | 46.45 | C |
| ATOM | 2844 | CD1 | LEU | C | 134 | 10.562 | −21.183 | 33.864 | 1.00 | 46.22 | C |
| ATOM | 2845 | CD2 | LEU | C | 134 | 9.995 | −22.041 | 36.149 | 1.00 | 46.70 | C |
| ATOM | 2846 | N | ARG | C | 135 | 5.231 | −21.576 | 35.169 | 1.00 | 45.51 | N |
| ATOM | 2847 | CA | ARG | C | 135 | 4.176 | −22.082 | 36.039 | 1.00 | 45.31 | C |
| ATOM | 2848 | C | ARG | C | 135 | 3.020 | −22.698 | 35.247 | 1.00 | 44.95 | C |
| ATOM | 2849 | O | ARG | C | 135 | 2.316 | −23.575 | 35.742 | 1.00 | 44.93 | O |
| ATOM | 2850 | CB | ARG | C | 135 | 3.682 | −20.958 | 36.958 | 1.00 | 45.27 | C |
| ATOM | 2851 | CG | ARG | C | 135 | 2.464 | −21.299 | 37.835 | 1.00 | 45.75 | C |
| ATOM | 2852 | CD | ARG | C | 135 | 2.128 | −20.172 | 38.811 | 1.00 | 45.59 | C |
| ATOM | 2853 | NE | ARG | C | 135 | 2.589 | −18.868 | 38.332 | 1.00 | 45.78 | N |
| ATOM | 2854 | CZ | ARG | C | 135 | 1.896 | −18.055 | 37.538 | 1.00 | 46.16 | C |
| ATOM | 2855 | NH1 | ARG | C | 135 | 0.681 | −18.391 | 37.117 | 1.00 | 45.68 | N |
| ATOM | 2856 | NH2 | ARG | C | 135 | 2.428 | −16.894 | 37.165 | 1.00 | 45.89 | N |
| ATOM | 2857 | N | LYS | C | 136 | 2.841 | −22.249 | 34.011 | 1.00 | 44.66 | N |
| ATOM | 2858 | CA | LYS | C | 136 | 1.711 | −22.693 | 33.197 | 1.00 | 44.30 | C |
| ATOM | 2859 | C | LYS | C | 136 | 2.056 | −23.809 | 32.197 | 1.00 | 44.11 | C |
| ATOM | 2860 | O | LYS | C | 136 | 1.164 | −24.470 | 31.670 | 1.00 | 43.94 | O |
| ATOM | 2861 | CB | LYS | C | 136 | 1.042 | −21.489 | 32.505 | 1.00 | 44.45 | C |
| ATOM | 2862 | CG | LYS | C | 136 | 0.357 | −20.537 | 33.498 | 1.00 | 44.18 | C |
| ATOM | 2863 | CD | LYS | C | 136 | −0.452 | −19.442 | 32.827 | 1.00 | 43.68 | C |
| ATOM | 2864 | CE | LYS | C | 136 | −1.215 | −18.624 | 33.864 | 1.00 | 42.16 | C |
| ATOM | 2865 | NZ | LYS | C | 136 | −1.905 | −17.457 | 33.252 | 1.00 | 41.41 | N |
| ATOM | 2866 | N | TYR | C | 137 | 3.346 | −24.021 | 31.950 | 1.00 | 43.90 | N |
| ATOM | 2867 | CA | TYR | C | 137 | 3.782 | −25.041 | 31.000 | 1.00 | 43.69 | C |
| ATOM | 2868 | C | TYR | C | 137 | 4.781 | −26.041 | 31.568 | 1.00 | 43.85 | C |
| ATOM | 2869 | O | TYR | C | 137 | 4.916 | −27.146 | 31.038 | 1.00 | 43.95 | O |
| ATOM | 2870 | CB | TYR | C | 137 | 4.344 | −24.398 | 29.732 | 1.00 | 43.46 | C |
| ATOM | 2871 | CG | TYR | C | 137 | 3.318 | −23.627 | 28.953 | 1.00 | 42.77 | C |
| ATOM | 2872 | CD1 | TYR | C | 137 | 2.416 | −24.281 | 28.118 | 1.00 | 41.89 | C |
| ATOM | 2873 | CD2 | TYR | C | 137 | 3.239 | −22.239 | 29.060 | 1.00 | 42.74 | C |
| ATOM | 2874 | CE1 | TYR | C | 137 | 1.460 | −23.571 | 27.402 | 1.00 | 42.38 | C |
| ATOM | 2875 | CE2 | TYR | C | 137 | 2.289 | −21.516 | 28.347 | 1.00 | 42.66 | C |
| ATOM | 2876 | CZ | TYR | C | 137 | 1.404 | −22.187 | 27.518 | 1.00 | 42.73 | C |
| ATOM | 2877 | OH | TYR | C | 137 | 0.468 | −21.473 | 26.811 | 1.00 | 42.76 | O |
| ATOM | 2878 | N | GLY | C | 138 | 5.474 | −25.656 | 32.638 | 1.00 | 44.05 | N |
| ATOM | 2879 | CA | GLY | C | 138 | 6.461 | −26.528 | 33.280 | 1.00 | 44.27 | C |
| ATOM | 2880 | C | GLY | C | 138 | 7.864 | −26.268 | 32.762 | 1.00 | 44.38 | C |
| ATOM | 2881 | O | GLY | C | 138 | 8.830 | −26.281 | 33.528 | 1.00 | 44.64 | O |
| ATOM | 2882 | N | ASN | C | 139 | 7.972 | −26.055 | 31.453 | 1.00 | 44.23 | N |
| ATOM | 2883 | CA | ASN | C | 139 | 9.210 | −25.610 | 30.834 | 1.00 | 44.15 | C |
| ATOM | 2884 | C | ASN | C | 139 | 9.115 | −24.112 | 30.541 | 1.00 | 44.42 | C |
| ATOM | 2885 | O | ASN | C | 139 | 8.298 | −23.405 | 31.144 | 1.00 | 44.57 | O |
| ATOM | 2886 | CB | ASN | C | 139 | 9.511 | −26.419 | 29.563 | 1.00 | 44.00 | C |
| ATOM | 2887 | CG | ASN | C | 139 | 8.382 | −26.363 | 28.543 | 1.00 | 43.83 | C |
| ATOM | 2888 | OD1 | ASN | C | 139 | 8.113 | −25.321 | 27.946 | 1.00 | 44.86 | O |
| ATOM | 2889 | ND2 | ASN | C | 139 | 7.727 | −27.492 | 28.329 | 1.00 | 43.09 | N |
| ATOM | 2890 | N | ALA | C | 140 | 9.939 | −23.631 | 29.616 | 1.00 | 44.36 | N |
| ATOM | 2891 | CA | ALA | C | 140 | 9.940 | −22.222 | 29.254 | 1.00 | 44.28 | C |
| ATOM | 2892 | C | ALA | C | 140 | 10.093 | −22.043 | 27.745 | 1.00 | 44.28 | C |
| ATOM | 2893 | O | ALA | C | 140 | 10.400 | −20.941 | 27.267 | 1.00 | 44.15 | O |
| ATOM | 2894 | CB | ALA | C | 140 | 11.039 | −21.487 | 30.008 | 1.00 | 44.32 | C |
| ATOM | 2895 | N | ASN | C | 141 | 9.876 | −23.135 | 27.007 | 1.00 | 44.16 | N |
| ATOM | 2896 | CA | ASN | C | 141 | 9.851 | −23.115 | 25.543 | 1.00 | 44.06 | C |
| ATOM | 2897 | C | ASN | C | 141 | 8.891 | −22.050 | 25.001 | 1.00 | 44.12 | C |
| ATOM | 2898 | O | ASN | C | 141 | 9.171 | −21.418 | 23.980 | 1.00 | 44.31 | O |
| ATOM | 2899 | CB | ASN | C | 141 | 9.470 | −24.492 | 24.985 | 1.00 | 44.02 | C |
| ATOM | 2900 | CG | ASN | C | 141 | 10.460 | −25.596 | 25.373 | 1.00 | 44.35 | C |
| ATOM | 2901 | OD1 | ASN | C | 141 | 11.569 | −25.332 | 25.839 | 1.00 | 45.14 | O |
| ATOM | 2902 | ND2 | ASN | C | 141 | 10.053 | −26.842 | 25.173 | 1.00 | 43.62 | N |
| ATOM | 2903 | N | VAL | C | 142 | 7.775 | −21.846 | 25.706 | 1.00 | 43.88 | N |
| ATOM | 2904 | CA | VAL | C | 142 | 6.751 | −20.871 | 25.322 | 1.00 | 43.67 | C |
| ATOM | 2905 | C | VAL | C | 142 | 7.264 | −19.424 | 25.431 | 1.00 | 43.75 | C |
| ATOM | 2906 | O | VAL | C | 142 | 6.883 | −18.548 | 24.640 | 1.00 | 43.63 | O |
| ATOM | 2907 | CB | VAL | C | 142 | 5.456 | −21.094 | 26.135 | 1.00 | 43.68 | C |
| ATOM | 2908 | CG1 | VAL | C | 142 | 4.543 | −19.881 | 26.081 | 1.00 | 43.79 | C |
| ATOM | 2909 | CG2 | VAL | C | 142 | 4.728 | −22.331 | 25.624 | 1.00 | 43.30 | C |
| ATOM | 2910 | N | TRP | C | 143 | 8.142 | −19.186 | 26.400 | 1.00 | 43.73 | N |
| ATOM | 2911 | CA | TRP | C | 143 | 8.853 | −17.917 | 26.485 | 1.00 | 43.53 | C |
| ATOM | 2912 | C | TRP | C | 143 | 9.752 | −17.737 | 25.267 | 1.00 | 43.52 | C |
| ATOM | 2913 | O | TRP | C | 143 | 9.870 | −16.630 | 24.750 | 1.00 | 43.55 | O |
| ATOM | 2914 | CB | TRP | C | 143 | 9.678 | −17.832 | 27.772 | 1.00 | 43.41 | C |
| ATOM | 2915 | CG | TRP | C | 143 | 10.531 | −16.603 | 27.841 | 1.00 | 43.51 | C |
| ATOM | 2916 | CD1 | TRP | C | 143 | 10.153 | −15.361 | 28.275 | 1.00 | 43.38 | C |
| ATOM | 2917 | CD2 | TRP | C | 143 | 11.907 | −16.491 | 27.455 | 1.00 | 43.50 | C |
| ATOM | 2918 | NE1 | TRP | C | 143 | 11.210 | −14.486 | 28.183 | 1.00 | 43.21 | N |
| ATOM | 2919 | CE2 | TRP | C | 143 | 12.299 | −15.153 | 27.684 | 1.00 | 43.07 | C |
| ATOM | 2920 | CE3 | TRP | C | 143 | 12.848 | −17.395 | 26.938 | 1.00 | 43.40 | C |
| ATOM | 2921 | CZ2 | TRP | C | 143 | 13.591 | −14.695 | 27.417 | 1.00 | 43.03 | C |
| ATOM | 2922 | CZ3 | TRP | C | 143 | 14.133 | −16.938 | 26.670 | 1.00 | 43.22 | C |
| ATOM | 2923 | CH2 | TRP | C | 143 | 14.492 | −15.599 | 26.912 | 1.00 | 43.34 | C |
| ATOM | 2924 | N | LYS | C | 144 | 10.373 | −18.830 | 24.814 | 1.00 | 43.47 | N |
| ATOM | 2925 | CA | LYS | C | 144 | 11.269 | −18.793 | 23.658 | 1.00 | 43.41 | C |
| ATOM | 2926 | C | LYS | C | 144 | 10.514 | −18.603 | 22.344 | 1.00 | 43.29 | C |
| ATOM | 2927 | O | LYS | C | 144 | 10.983 | −17.886 | 21.459 | 1.00 | 43.18 | O |
| ATOM | 2928 | CB | LYS | C | 144 | 12.151 | −20.047 | 23.592 | 1.00 | 43.51 | C |
| ATOM | 2929 | CG | LYS | C | 144 | 13.417 | −19.874 | 22.746 | 1.00 | 44.28 | C |
| ATOM | 2930 | CD | LYS | C | 144 | 14.348 | −18.850 | 23.390 | 1.00 | 46.15 | C |
| ATOM | 2931 | CE | LYS | C | 144 | 14.832 | −17.801 | 22.390 | 1.00 | 46.82 | C |
| ATOM | 2932 | NZ | LYS | C | 144 | 14.735 | −16.407 | 22.943 | 1.00 | 46.01 | N |
| ATOM | 2933 | N | TYR | C | 145 | 9.351 | −19.244 | 22.224 | 1.00 | 43.16 | N |
| ATOM | 2934 | CA | TYR | C | 145 | 8.519 | −19.133 | 21.022 | 1.00 | 43.05 | C |
| ATOM | 2935 | C | TYR | C | 145 | 7.914 | −17.731 | 20.861 | 1.00 | 43.20 | C |
| ATOM | 2936 | O | TYR | C | 145 | 7.695 | −17.266 | 19.741 | 1.00 | 43.41 | O |
| ATOM | 2937 | CB | TYR | C | 145 | 7.395 | −20.176 | 21.018 | 1.00 | 42.93 | C |
| ATOM | 2938 | CG | TYR | C | 145 | 7.826 | −21.625 | 21.163 | 1.00 | 42.84 | C |
| ATOM | 2939 | CD1 | TYR | C | 145 | 7.034 | −22.530 | 21.870 | 1.00 | 42.76 | C |
| ATOM | 2940 | CD2 | TYR | C | 145 | 9.012 | −22.093 | 20.592 | 1.00 | 42.68 | C |
| ATOM | 2941 | CE1 | TYR | C | 145 | 7.408 | −23.867 | 22.012 | 1.00 | 42.70 | C |
| ATOM | 2942 | CE2 | TYR | C | 145 | 9.396 | −23.424 | 20.727 | 1.00 | 43.14 | C |
| ATOM | 2943 | CZ | TYR | C | 145 | 8.591 | −24.311 | 21.440 | 1.00 | 43.23 | C |
| ATOM | 2944 | OH | TYR | C | 145 | 8.966 | −25.640 | 21.579 | 1.00 | 42.61 | O |
| ATOM | 2945 | N | PHE | C | 146 | 7.638 | −17.063 | 21.975 | 1.00 | 43.13 | N |
| ATOM | 2946 | CA | PHE | C | 146 | 7.108 | −15.706 | 21.919 | 1.00 | 43.03 | C |
| ATOM | 2947 | C | PHE | C | 146 | 8.188 | −14.639 | 21.747 | 1.00 | 42.85 | C |
| ATOM | 2948 | O | PHE | C | 146 | 7.966 | −13.649 | 21.053 | 1.00 | 42.81 | O |
| ATOM | 2949 | CB | PHE | C | 146 | 6.231 | −15.404 | 23.137 | 1.00 | 43.09 | C |
| ATOM | 2950 | CG | PHE | C | 146 | 4.835 | −15.942 | 23.024 | 1.00 | 42.90 | C |
| ATOM | 2951 | CD1 | PHE | C | 146 | 3.985 | −15.502 | 22.011 | 1.00 | 42.82 | C |
| ATOM | 2952 | CD2 | PHE | C | 146 | 4.362 | −16.876 | 23.934 | 1.00 | 43.59 | C |
| ATOM | 2953 | CE1 | PHE | C | 146 | 2.689 | −15.989 | 21.900 | 1.00 | 42.79 | C |
| ATOM | 2954 | CE2 | PHE | C | 146 | 3.062 | −17.373 | 23.839 | 1.00 | 43.81 | C |
| ATOM | 2955 | CZ | PHE | C | 146 | 2.224 | −16.929 | 22.816 | 1.00 | 43.70 | C |
| ATOM | 2956 | N | THR | C | 147 | 9.349 | −14.835 | 22.366 | 1.00 | 42.59 | N |
| ATOM | 2957 | CA | THR | C | 147 | 10.418 | −13.842 | 22.258 | 1.00 | 42.63 | C |
| ATOM | 2958 | C | THR | C | 147 | 11.129 | −13.898 | 20.912 | 1.00 | 42.63 | C |
| ATOM | 2959 | O | THR | C | 147 | 11.603 | −12.869 | 20.416 | 1.00 | 42.84 | O |
| ATOM | 2960 | CB | THR | C | 147 | 11.439 | −13.900 | 23.417 | 1.00 | 42.56 | C |
| ATOM | 2961 | OG1 | THR | C | 147 | 11.866 | −15.252 | 23.638 | 1.00 | 42.99 | O |
| ATOM | 2962 | CG2 | THR | C | 147 | 10.828 | −13.328 | 24.688 | 1.00 | 42.52 | C |
| ATOM | 2963 | N | ASP | C | 148 | 11.201 | −15.093 | 20.326 | 1.00 | 42.39 | N |
| ATOM | 2964 | CA | ASP | C | 148 | 11.670 | −15.238 | 18.953 | 1.00 | 42.21 | C |
| ATOM | 2965 | C | ASP | C | 148 | 10.686 | −14.561 | 18.011 | 1.00 | 42.06 | C |
| ATOM | 2966 | O | ASP | C | 148 | 11.080 | −14.020 | 16.986 | 1.00 | 42.23 | O |
| ATOM | 2967 | CB | ASP | C | 148 | 11.842 | −16.717 | 18.568 | 1.00 | 42.20 | C |
| ATOM | 2968 | CG | ASP | C | 148 | 13.192 | −17.290 | 18.991 | 1.00 | 42.16 | C |
| ATOM | 2969 | OD1 | ASP | C | 148 | 14.045 | −16.533 | 19.508 | 1.00 | 42.42 | O |
| ATOM | 2970 | OD2 | ASP | C | 148 | 13.406 | −18.505 | 18.804 | 1.00 | 41.73 | O |
| ATOM | 2971 | N | LEU | C | 149 | 9.407 | −14.584 | 18.375 | 1.00 | 41.93 | N |
| ATOM | 2972 | CA | LEU | C | 149 | 8.360 | −13.980 | 17.563 | 1.00 | 42.03 | C |
| ATOM | 2973 | C | LEU | C | 149 | 8.385 | −12.462 | 17.653 | 1.00 | 42.12 | C |
| ATOM | 2974 | O | LEU | C | 149 | 8.177 | −11.775 | 16.656 | 1.00 | 41.99 | O |
| ATOM | 2975 | CB | LEU | C | 149 | 6.982 | −14.501 | 17.982 | 1.00 | 41.99 | C |
| ATOM | 2976 | CG | LEU | C | 149 | 5.765 | −13.943 | 17.237 | 1.00 | 42.31 | C |
| ATOM | 2977 | CD1 | LEU | C | 149 | 5.863 | −14.253 | 15.746 | 1.00 | 43.35 | C |
| ATOM | 2978 | CD2 | LEU | C | 149 | 4.462 | −14.485 | 17.814 | 1.00 | 42.37 | C |
| ATOM | 2979 | N | PHE | C | 150 | 8.637 | −11.946 | 18.855 | 1.00 | 42.27 | N |
| ATOM | 2980 | CA | PHE | C | 150 | 8.633 | −10.515 | 19.083 | 1.00 | 42.26 | C |
| ATOM | 2981 | C | PHE | C | 150 | 9.612 | −9.816 | 18.154 | 1.00 | 42.49 | C |
| ATOM | 2982 | O | PHE | C | 150 | 9.377 | −8.684 | 17.745 | 1.00 | 42.64 | O |
| ATOM | 2983 | CB | PHE | C | 150 | 8.939 | −10.185 | 20.547 | 1.00 | 42.36 | C |
| ATOM | 2984 | CG | PHE | C | 150 | 7.885 | −10.657 | 21.528 | 1.00 | 41.76 | C |
| ATOM | 2985 | CD1 | PHE | C | 150 | 6.555 | −10.796 | 21.149 | 1.00 | 41.38 | C |
| ATOM | 2986 | CD2 | PHE | C | 150 | 8.230 | −10.932 | 22.848 | 1.00 | 41.27 | C |
| ATOM | 2987 | CE1 | PHE | C | 150 | 5.587 | −11.233 | 22.073 | 1.00 | 42.25 | C |
| ATOM | 2988 | CE2 | PHE | C | 150 | 7.273 | −11.360 | 23.776 | 1.00 | 41.13 | C |
| ATOM | 2989 | CZ | PHE | C | 150 | 5.950 | −11.512 | 23.390 | 1.00 | 41.26 | C |
| ATOM | 2990 | N | ASP | C | 151 | 10.692 | −10.513 | 17.800 | 1.00 | 42.91 | N |
| ATOM | 2991 | CA | ASP | C | 151 | 11.695 | −10.011 | 16.848 | 1.00 | 43.16 | C |
| ATOM | 2992 | C | ASP | C | 151 | 11.117 | −9.649 | 15.476 | 1.00 | 43.41 | C |
| ATOM | 2993 | O | ASP | C | 151 | 11.734 | −8.909 | 14.710 | 1.00 | 43.42 | O |
| ATOM | 2994 | CB | ASP | C | 151 | 12.837 | −11.024 | 16.693 | 1.00 | 42.87 | C |
| ATOM | 2995 | CG | ASP | C | 151 | 13.690 | −11.140 | 17.942 | 1.00 | 42.84 | C |
| ATOM | 2996 | OD1 | ASP | C | 151 | 13.316 | −10.552 | 18.973 | 1.00 | 43.62 | O |
| ATOM | 2997 | OD2 | ASP | C | 151 | 14.740 | −11.812 | 17.903 | 1.00 | 42.40 | O |
| ATOM | 2998 | N | TYR | C | 152 | 9.926 | −10.169 | 15.187 | 1.00 | 43.89 | N |
| ATOM | 2999 | CA | TYR | C | 152 | 9.284 | −10.027 | 13.875 | 1.00 | 43.96 | C |
| ATOM | 3000 | C | TYR | C | 152 | 8.193 | −8.957 | 13.853 | 1.00 | 43.91 | C |
| ATOM | 3001 | O | TYR | C | 152 | 7.788 | −8.507 | 12.776 | 1.00 | 44.15 | O |
| ATOM | 3002 | CB | TYR | C | 152 | 8.720 | −11.376 | 13.412 | 1.00 | 43.95 | C |
| ATOM | 3003 | CG | TYR | C | 152 | 9.768 | −12.293 | 12.847 | 1.00 | 44.24 | C |
| ATOM | 3004 | CD1 | TYR | C | 152 | 10.545 | −13.100 | 13.678 | 1.00 | 44.06 | C |
| ATOM | 3005 | CD2 | TYR | C | 152 | 9.992 | −12.350 | 11.468 | 1.00 | 45.46 | C |
| ATOM | 3006 | CE1 | TYR | C | 152 | 11.526 | −13.943 | 13.146 | 1.00 | 44.76 | C |
| ATOM | 3007 | CE2 | TYR | C | 152 | 10.967 | −13.183 | 10.923 | 1.00 | 44.97 | C |
| ATOM | 3008 | CZ | TYR | C | 152 | 11.730 | −13.976 | 11.762 | 1.00 | 44.93 | C |
| ATOM | 3009 | OH | TYR | C | 152 | 12.688 | −14.797 | 11.208 | 1.00 | 44.36 | O |
| ATOM | 3010 | N | LEU | C | 153 | 7.717 | −8.552 | 15.029 | 1.00 | 43.67 | N |
| ATOM | 3011 | CA | LEU | C | 153 | 6.725 | −7.482 | 15.112 | 1.00 | 43.68 | C |
| ATOM | 3012 | C | LEU | C | 153 | 7.240 | −6.243 | 14.389 | 1.00 | 43.85 | C |
| ATOM | 3013 | O | LEU | C | 153 | 8.408 | −5.880 | 14.541 | 1.00 | 44.15 | O |
| ATOM | 3014 | CB | LEU | C | 153 | 6.409 | −7.127 | 16.565 | 1.00 | 43.48 | C |
| ATOM | 3015 | CG | LEU | C | 153 | 5.720 | −8.167 | 17.448 | 1.00 | 43.09 | C |
| ATOM | 3016 | CD1 | LEU | C | 153 | 5.878 | −7.823 | 18.912 | 1.00 | 42.26 | C |
| ATOM | 3017 | CD2 | LEU | C | 153 | 4.262 | −8.262 | 17.094 | 1.00 | 43.18 | C |
| ATOM | 3018 | N | PRO | C | 154 | 6.376 | −5.585 | 13.599 | 1.00 | 43.88 | N |
| ATOM | 3019 | CA | PRO | C | 154 | 6.812 | −4.342 | 12.982 | 1.00 | 43.84 | C |
| ATOM | 3020 | C | PRO | C | 154 | 7.006 | −3.311 | 14.079 | 1.00 | 43.77 | C |
| ATOM | 3021 | O | PRO | C | 154 | 6.408 | −3.442 | 15.151 | 1.00 | 43.74 | O |
| ATOM | 3022 | CB | PRO | C | 154 | 5.630 | −3.945 | 12.096 | 1.00 | 44.01 | C |
| ATOM | 3023 | CG | PRO | C | 154 | 4.743 | −5.145 | 12.028 | 1.00 | 43.87 | C |
| ATOM | 3024 | CD | PRO | C | 154 | 4.979 | −5.910 | 13.268 | 1.00 | 43.87 | C |
| ATOM | 3025 | N | LEU | C | 155 | 7.839 | −2.309 | 13.816 | 1.00 | 43.82 | N |
| ATOM | 3026 | CA | LEU | C | 155 | 8.196 | −1.304 | 14.816 | 1.00 | 43.81 | C |
| ATOM | 3027 | C | LEU | C | 155 | 7.208 | −0.148 | 14.823 | 1.00 | 43.90 | C |
| ATOM | 3028 | O | LEU | C | 155 | 6.772 | 0.315 | 15.888 | 1.00 | 43.97 | O |
| ATOM | 3029 | CB | LEU | C | 155 | 9.596 | −0.765 | 14.543 | 1.00 | 43.76 | C |
| ATOM | 3030 | CG | LEU | C | 155 | 10.737 | −1.769 | 14.397 | 1.00 | 43.53 | C |
| ATOM | 3031 | CD1 | LEU | C | 155 | 11.970 | −1.071 | 13.825 | 1.00 | 42.27 | C |
| ATOM | 3032 | CD2 | LEU | C | 155 | 11.037 | −2.438 | 15.739 | 1.00 | 42.92 | C |
| ATOM | 3033 | N | THR | C | 156 | 6.877 | 0.320 | 13.624 | 1.00 | 43.80 | N |
| ATOM | 3034 | CA | THR | C | 156 | 5.957 | 1.437 | 13.445 | 1.00 | 43.81 | C |
| ATOM | 3035 | C | THR | C | 156 | 4.854 | 1.068 | 12.448 | 1.00 | 43.90 | C |
| ATOM | 3036 | O | THR | C | 156 | 4.893 | −0.003 | 11.830 | 1.00 | 44.03 | O |
| ATOM | 3037 | CB | THR | C | 156 | 6.687 | 2.708 | 12.939 | 1.00 | 43.78 | C |
| ATOM | 3038 | OG1 | THR | C | 156 | 7.183 | 2.483 | 11.612 | 1.00 | 43.51 | O |
| ATOM | 3039 | CG2 | THR | C | 156 | 7.842 | 3.101 | 13.867 | 1.00 | 43.28 | C |
| ATOM | 3040 | N | ALA | C | 157 | 3.878 | 1.961 | 12.302 | 1.00 | 43.65 | N |
| ATOM | 3041 | CA | ALA | C | 157 | 2.775 | 1.786 | 11.371 | 1.00 | 43.44 | C |
| ATOM | 3042 | C | ALA | C | 157 | 2.306 | 3.150 | 10.922 | 1.00 | 43.56 | C |
| ATOM | 3043 | O | ALA | C | 157 | 2.449 | 4.133 | 11.646 | 1.00 | 43.62 | O |
| ATOM | 3044 | CB | ALA | C | 157 | 1.639 | 1.038 | 12.020 | 1.00 | 43.36 | C |
| ATOM | 3045 | N | LEU | C | 158 | 1.742 | 3.206 | 9.722 | 1.00 | 43.77 | N |
| ATOM | 3046 | CA | LEU | C | 158 | 1.294 | 4.459 | 9.147 | 1.00 | 43.89 | C |
| ATOM | 3047 | C | LEU | C | 158 | −0.139 | 4.322 | 8.645 | 1.00 | 44.05 | C |
| ATOM | 3048 | O | LEU | C | 158 | −0.420 | 3.533 | 7.742 | 1.00 | 44.22 | O |
| ATOM | 3049 | CB | LEU | C | 158 | 2.246 | 4.875 | 8.024 | 1.00 | 44.01 | C |
| ATOM | 3050 | CG | LEU | C | 158 | 1.980 | 6.167 | 7.249 | 1.00 | 44.31 | C |
| ATOM | 3051 | CD1 | LEU | C | 158 | 1.957 | 7.367 | 8.186 | 1.00 | 44.04 | C |
| ATOM | 3052 | CD2 | LEU | C | 158 | 3.007 | 6.343 | 6.122 | 1.00 | 43.61 | C |
| ATOM | 3053 | N | VAL | C | 159 | −1.043 | 5.082 | 9.252 | 1.00 | 44.27 | N |
| ATOM | 3054 | CA | VAL | C | 159 | −2.460 | 5.018 | 8.909 | 1.00 | 44.36 | C |
| ATOM | 3055 | C | VAL | C | 159 | −2.755 | 6.034 | 7.810 | 1.00 | 44.75 | C |
| ATOM | 3056 | O | VAL | C | 159 | −2.601 | 7.243 | 8.009 | 1.00 | 44.88 | O |
| ATOM | 3057 | CB | VAL | C | 159 | −3.368 | 5.234 | 10.154 | 1.00 | 44.29 | C |
| ATOM | 3058 | CG1 | VAL | C | 159 | −4.831 | 5.070 | 9.792 | 1.00 | 43.85 | C |
| ATOM | 3059 | CG2 | VAL | C | 159 | −2.997 | 4.253 | 11.246 | 1.00 | 43.48 | C |
| ATOM | 3060 | N | ASP | C | 160 | −3.153 | 5.515 | 6.647 | 1.00 | 45.21 | N |
| ATOM | 3061 | CA | ASP | C | 160 | −3.424 | 6.298 | 5.426 | 1.00 | 45.46 | C |
| ATOM | 3062 | C | ASP | C | 160 | −2.394 | 7.379 | 5.085 | 1.00 | 45.40 | C |
| ATOM | 3063 | O | ASP | C | 160 | −2.755 | 8.475 | 4.662 | 1.00 | 45.17 | O |
| ATOM | 3064 | CB | ASP | C | 160 | −4.846 | 6.870 | 5.454 | 1.00 | 45.67 | C |
| ATOM | 3065 | CG | ASP | C | 160 | −5.880 | 5.865 | 4.987 | 1.00 | 46.41 | C |
| ATOM | 3066 | OD1 | ASP | C | 160 | −6.247 | 4.972 | 5.778 | 1.00 | 46.67 | O |
| ATOM | 3067 | OD2 | ASP | C | 160 | −6.320 | 5.965 | 3.821 | 1.00 | 46.76 | O |
| ATOM | 3068 | N | GLY | C | 161 | −1.117 | 7.060 | 5.280 | 1.00 | 45.66 | N |
| ATOM | 3069 | CA | GLY | C | 161 | −0.017 | 7.988 | 5.001 | 1.00 | 45.98 | C |
| ATOM | 3070 | C | GLY | C | 161 | −0.007 | 9.301 | 5.770 | 1.00 | 46.29 | C |
| ATOM | 3071 | O | GLY | C | 161 | 0.660 | 10.252 | 5.360 | 1.00 | 46.37 | O |
| ATOM | 3072 | N | GLN | C | 162 | −0.727 | 9.358 | 6.890 | 1.00 | 46.50 | N |
| ATOM | 3073 | CA | GLN | C | 162 | −0.932 | 10.618 | 7.608 | 1.00 | 46.74 | C |
| ATOM | 3074 | C | GLN | C | 162 | −0.659 | 10.521 | 9.098 | 1.00 | 46.80 | C |
| ATOM | 3075 | O | GLN | C | 162 | −0.023 | 11.412 | 9.673 | 1.00 | 47.08 | O |
| ATOM | 3076 | CB | GLN | C | 162 | −2.366 | 11.102 | 7.435 | 1.00 | 46.88 | C |
| ATOM | 3077 | CG | GLN | C | 162 | −2.690 | 11.719 | 6.105 | 1.00 | 47.26 | C |
| ATOM | 3078 | CD | GLN | C | 162 | −4.135 | 12.151 | 6.045 | 1.00 | 48.46 | C |
| ATOM | 3079 | OE1 | GLN | C | 162 | −4.580 | 12.981 | 6.843 | 1.00 | 49.04 | O |
| ATOM | 3080 | NE2 | GLN | C | 162 | −4.886 | 11.580 | 5.110 | 1.00 | 48.90 | N |
| ATOM | 3081 | N | ILE | C | 163 | −1.169 | 9.460 | 9.722 | 1.00 | 46.56 | N |
| ATOM | 3082 | CA | ILE | C | 163 | −1.034 | 9.274 | 11.164 | 1.00 | 46.23 | C |
| ATOM | 3083 | C | ILE | C | 163 | −0.012 | 8.181 | 11.471 | 1.00 | 45.91 | C |
| ATOM | 3084 | O | ILE | C | 163 | −0.164 | 7.031 | 11.049 | 1.00 | 45.88 | O |
| ATOM | 3085 | CB | ILE | C | 163 | −2.403 | 9.004 | 11.832 | 1.00 | 46.41 | C |
| ATOM | 3086 | CG1 | ILE | C | 163 | −3.339 | 10.199 | 11.585 | 1.00 | 46.38 | C |
| ATOM | 3087 | CG2 | ILE | C | 163 | −2.238 | 8.694 | 13.343 | 1.00 | 46.22 | C |
| ATOM | 3088 | CD1 | ILE | C | 163 | −4.787 | 9.978 | 12.010 | 1.00 | 46.42 | C |
| ATOM | 3089 | N | PHE | C | 164 | 1.025 | 8.568 | 12.212 | 1.00 | 45.36 | N |
| ATOM | 3090 | CA | PHE | C | 164 | 2.183 | 7.728 | 12.457 | 1.00 | 44.97 | C |
| ATOM | 3091 | C | PHE | C | 164 | 2.095 | 7.070 | 13.833 | 1.00 | 44.94 | C |
| ATOM | 3092 | O | PHE | C | 164 | 1.869 | 7.747 | 14.837 | 1.00 | 45.10 | O |
| ATOM | 3093 | CB | PHE | C | 164 | 3.452 | 8.574 | 12.313 | 1.00 | 44.92 | C |
| ATOM | 3094 | CG | PHE | C | 164 | 4.722 | 7.835 | 12.607 | 1.00 | 44.82 | C |
| ATOM | 3095 | CD1 | PHE | C | 164 | 5.081 | 6.718 | 11.874 | 1.00 | 44.47 | C |
| ATOM | 3096 | CD2 | PHE | C | 164 | 5.570 | 8.272 | 13.620 | 1.00 | 45.04 | C |
| ATOM | 3097 | CE1 | PHE | C | 164 | 6.257 | 6.035 | 12.159 | 1.00 | 44.80 | C |
| ATOM | 3098 | CE2 | PHE | C | 164 | 6.755 | 7.601 | 13.904 | 1.00 | 44.83 | C |
| ATOM | 3099 | CZ | PHE | C | 164 | 7.098 | 6.480 | 13.175 | 1.00 | 44.85 | C |
| ATOM | 3100 | N | CYS | C | 165 | 2.273 | 5.750 | 13.873 | 1.00 | 44.53 | N |
| ATOM | 3101 | CA | CYS | C | 165 | 2.045 | 4.980 | 15.093 | 1.00 | 44.20 | C |
| ATOM | 3102 | C | CYS | C | 165 | 3.242 | 4.162 | 15.531 | 1.00 | 44.07 | C |
| ATOM | 3103 | O | CYS | C | 165 | 3.894 | 3.515 | 14.723 | 1.00 | 44.18 | O |
| ATOM | 3104 | CB | CYS | C | 165 | 0.842 | 4.057 | 14.920 | 1.00 | 44.33 | C |
| ATOM | 3105 | SG | CYS | C | 165 | −0.680 | 4.920 | 14.536 | 1.00 | 43.92 | S |
| ATOM | 3106 | N | LEU | C | 166 | 3.501 | 4.166 | 16.830 | 1.00 | 43.89 | N |
| ATOM | 3107 | CA | LEU | C | 166 | 4.648 | 3.469 | 17.392 | 1.00 | 43.95 | C |
| ATOM | 3108 | C | LEU | C | 166 | 4.483 | 3.438 | 18.894 | 1.00 | 44.26 | C |
| ATOM | 3109 | O | LEU | C | 166 | 3.707 | 4.215 | 19.443 | 1.00 | 44.98 | O |
| ATOM | 3110 | CB | LEU | C | 166 | 5.949 | 4.194 | 17.023 | 1.00 | 43.87 | C |
| ATOM | 3111 | CG | LEU | C | 166 | 6.307 | 5.561 | 17.633 | 1.00 | 42.79 | C |
| ATOM | 3112 | CD1 | LEU | C | 166 | 7.694 | 5.938 | 17.178 | 1.00 | 41.36 | C |
| ATOM | 3113 | CD2 | LEU | C | 166 | 5.314 | 6.669 | 17.292 | 1.00 | 41.00 | C |
| ATOM | 3114 | N | HIS | C | 167 | 5.216 | 2.565 | 19.570 | 1.00 | 44.28 | N |
| ATOM | 3115 | CA | HIS | C | 167 | 5.101 | 2.487 | 21.015 | 1.00 | 44.24 | C |
| ATOM | 3116 | C | HIS | C | 167 | 5.655 | 3.712 | 21.746 | 1.00 | 44.31 | C |
| ATOM | 3117 | O | HIS | C | 167 | 4.922 | 4.371 | 22.484 | 1.00 | 44.61 | O |
| ATOM | 3118 | CB | HIS | C | 167 | 5.737 | 1.217 | 21.564 | 1.00 | 44.06 | C |
| ATOM | 3119 | CG | HIS | C | 167 | 5.418 | 0.982 | 23.002 | 1.00 | 44.31 | C |
| ATOM | 3120 | ND1 | HIS | C | 167 | 6.372 | 1.031 | 23.994 | 1.00 | 45.49 | N |
| ATOM | 3121 | CD2 | HIS | C | 167 | 4.238 | 0.743 | 23.624 | 1.00 | 44.45 | C |
| ATOM | 3122 | CE1 | HIS | C | 167 | 5.796 | 0.807 | 25.163 | 1.00 | 46.03 | C |
| ATOM | 3123 | NE2 | HIS | C | 167 | 4.500 | 0.636 | 24.966 | 1.00 | 45.04 | N |
| ATOM | 3124 | N | GLY | C | 168 | 6.932 | 4.018 | 21.534 | 1.00 | 44.16 | N |
| ATOM | 3125 | CA | GLY | C | 168 | 7.610 | 5.061 | 22.298 | 1.00 | 43.95 | C |
| ATOM | 3126 | C | GLY | C | 168 | 7.562 | 6.448 | 21.678 | 1.00 | 44.08 | C |
| ATOM | 3127 | O | GLY | C | 168 | 6.684 | 7.269 | 22.010 | 1.00 | 43.93 | O |
| ATOM | 3128 | N | GLY | C | 169 | 8.524 | 6.715 | 20.793 | 1.00 | 43.93 | N |
| ATOM | 3129 | CA | GLY | C | 169 | 8.639 | 8.018 | 20.148 | 1.00 | 43.80 | C |
| ATOM | 3130 | C | GLY | C | 169 | 9.853 | 8.209 | 19.248 | 1.00 | 43.84 | C |
| ATOM | 3131 | O | GLY | C | 169 | 10.457 | 7.241 | 18.766 | 1.00 | 43.67 | O |
| ATOM | 3132 | N | LEU | C | 170 | 10.217 | 9.475 | 19.054 | 1.00 | 43.53 | N |
| ATOM | 3133 | CA | LEU | C | 170 | 11.185 | 9.879 | 18.046 | 1.00 | 43.37 | C |
| ATOM | 3134 | C | LEU | C | 170 | 12.647 | 9.626 | 18.445 | 1.00 | 43.30 | C |
| ATOM | 3135 | O | LEU | C | 170 | 12.959 | 9.452 | 19.623 | 1.00 | 43.11 | O |
| ATOM | 3136 | CB | LEU | C | 170 | 10.933 | 11.340 | 17.652 | 1.00 | 43.44 | C |
| ATOM | 3137 | CG | LEU | C | 170 | 9.474 | 11.698 | 17.289 | 1.00 | 43.81 | C |
| ATOM | 3138 | CD1 | LEU | C | 170 | 9.295 | 13.191 | 17.021 | 1.00 | 43.07 | C |
| ATOM | 3139 | CD2 | LEU | C | 170 | 8.921 | 10.863 | 16.110 | 1.00 | 43.45 | C |
| ATOM | 3140 | N | SER | C | 171 | 13.522 | 9.589 | 17.441 | 1.00 | 43.35 | N |
| ATOM | 3141 | CA | SER | C | 171 | 14.945 | 9.288 | 17.612 | 1.00 | 43.46 | C |
| ATOM | 3142 | C | SER | C | 171 | 15.803 | 10.333 | 16.924 | 1.00 | 43.35 | C |
| ATOM | 3143 | O | SER | C | 171 | 15.440 | 10.805 | 15.846 | 1.00 | 43.36 | O |
| ATOM | 3144 | CB | SER | C | 171 | 15.291 | 7.912 | 17.022 | 1.00 | 43.63 | C |
| ATOM | 3145 | OG | SER | C | 171 | 16.704 | 7.713 | 16.940 | 1.00 | 43.29 | O |
| ATOM | 3146 | N | PRO | C | 172 | 16.956 | 10.686 | 17.535 | 1.00 | 43.29 | N |
| ATOM | 3147 | CA | PRO | C | 172 | 17.924 | 11.586 | 16.890 | 1.00 | 43.06 | C |
| ATOM | 3148 | C | PRO | C | 172 | 18.640 | 10.923 | 15.707 | 1.00 | 42.92 | C |
| ATOM | 3149 | O | PRO | C | 172 | 19.400 | 11.584 | 14.999 | 1.00 | 42.97 | O |
| ATOM | 3150 | CB | PRO | C | 172 | 18.916 | 11.902 | 18.014 | 1.00 | 42.66 | C |
| ATOM | 3151 | CG | PRO | C | 172 | 18.838 | 10.733 | 18.918 | 1.00 | 43.13 | C |
| ATOM | 3152 | CD | PRO | C | 172 | 17.410 | 10.264 | 18.876 | 1.00 | 43.27 | C |
| ATOM | 3153 | N | SER | C | 173 | 18.382 | 9.632 | 15.504 | 1.00 | 42.83 | N |
| ATOM | 3154 | CA | SER | C | 173 | 18.980 | 8.862 | 14.419 | 1.00 | 42.65 | C |
| ATOM | 3155 | C | SER | C | 173 | 18.004 | 8.614 | 13.273 | 1.00 | 42.65 | C |
| ATOM | 3156 | O | SER | C | 173 | 18.357 | 7.961 | 12.291 | 1.00 | 42.75 | O |
| ATOM | 3157 | CB | SER | C | 173 | 19.477 | 7.522 | 14.949 | 1.00 | 42.61 | C |
| ATOM | 3158 | OG | SER | C | 173 | 20.436 | 7.721 | 15.962 | 1.00 | 42.45 | O |
| ATOM | 3159 | N | ILE | C | 174 | 16.781 | 9.122 | 13.410 | 1.00 | 42.59 | N |
| ATOM | 3160 | CA | ILE | C | 174 | 15.750 | 8.964 | 12.386 | 1.00 | 42.63 | C |
| ATOM | 3161 | C | ILE | C | 174 | 15.064 | 10.291 | 12.071 | 1.00 | 42.72 | C |
| ATOM | 3162 | O | ILE | C | 174 | 14.435 | 10.904 | 12.937 | 1.00 | 42.53 | O |
| ATOM | 3163 | CB | ILE | C | 174 | 14.720 | 7.865 | 12.778 | 1.00 | 42.78 | C |
| ATOM | 3164 | CG1 | ILE | C | 174 | 15.326 | 6.485 | 12.526 | 1.00 | 42.72 | C |
| ATOM | 3165 | CG2 | ILE | C | 174 | 13.406 | 8.013 | 11.995 | 1.00 | 42.12 | C |
| ATOM | 3166 | CD1 | ILE | C | 174 | 14.846 | 5.448 | 13.464 | 1.00 | 43.77 | C |
| ATOM | 3167 | N | ASP | C | 175 | 15.209 | 10.724 | 10.822 | 1.00 | 42.84 | N |
| ATOM | 3168 | CA | ASP | C | 175 | 14.576 | 11.947 | 10.337 | 1.00 | 43.03 | C |
| ATOM | 3169 | C | ASP | C | 175 | 13.354 | 11.610 | 9.491 | 1.00 | 42.97 | C |
| ATOM | 3170 | O | ASP | C | 175 | 12.320 | 12.274 | 9.584 | 1.00 | 42.99 | O |
| ATOM | 3171 | CB | ASP | C | 175 | 15.557 | 12.767 | 9.502 | 1.00 | 43.07 | C |
| ATOM | 3172 | CG | ASP | C | 175 | 16.845 | 13.069 | 10.237 | 1.00 | 43.71 | C |
| ATOM | 3173 | OD1 | ASP | C | 175 | 16.870 | 14.053 | 11.004 | 1.00 | 44.97 | O |
| ATOM | 3174 | OD2 | ASP | C | 175 | 17.840 | 12.337 | 10.033 | 1.00 | 44.29 | O |
| ATOM | 3175 | N | THR | C | 176 | 13.484 | 10.569 | 8.671 | 1.00 | 42.92 | N |
| ATOM | 3176 | CA | THR | C | 176 | 12.447 | 10.192 | 7.709 | 1.00 | 42.76 | C |
| ATOM | 3177 | C | THR | C | 176 | 11.925 | 8.782 | 7.960 | 1.00 | 42.63 | C |
| ATOM | 3178 | O | THR | C | 176 | 12.576 | 7.985 | 8.634 | 1.00 | 42.66 | O |
| ATOM | 3179 | CB | THR | C | 176 | 12.962 | 10.271 | 6.240 | 1.00 | 42.71 | C |
| ATOM | 3180 | OG1 | THR | C | 176 | 13.761 | 9.119 | 5.941 | 1.00 | 42.38 | O |
| ATOM | 3181 | CG2 | THR | C | 176 | 13.778 | 11.540 | 6.002 | 1.00 | 42.44 | C |
| ATOM | 3182 | N | LEU | C | 177 | 10.757 | 8.483 | 7.398 | 1.00 | 42.66 | N |
| ATOM | 3183 | CA | LEU | C | 177 | 10.199 | 7.133 | 7.413 | 1.00 | 42.77 | C |
| ATOM | 3184 | C | LEU | C | 177 | 11.111 | 6.104 | 6.736 | 1.00 | 42.94 | C |
| ATOM | 3185 | O | LEU | C | 177 | 11.195 | 4.961 | 7.195 | 1.00 | 43.18 | O |
| ATOM | 3186 | CB | LEU | C | 177 | 8.814 | 7.115 | 6.768 | 1.00 | 42.70 | C |
| ATOM | 3187 | CG | LEU | C | 177 | 7.693 | 7.893 | 7.463 | 1.00 | 43.17 | C |
| ATOM | 3188 | CD1 | LEU | C | 177 | 6.491 | 7.982 | 6.546 | 1.00 | 44.06 | C |
| ATOM | 3189 | CD2 | LEU | C | 177 | 7.292 | 7.274 | 8.800 | 1.00 | 43.08 | C |
| ATOM | 3190 | N | ASP | C | 178 | 11.791 | 6.512 | 5.659 | 1.00 | 42.89 | N |
| ATOM | 3191 | CA | ASP | C | 178 | 12.777 | 5.666 | 4.978 | 1.00 | 42.91 | C |
| ATOM | 3192 | C | ASP | C | 178 | 13.929 | 5.205 | 5.874 | 1.00 | 42.84 | C |
| ATOM | 3193 | O | ASP | C | 178 | 14.463 | 4.104 | 5.684 | 1.00 | 42.67 | O |
| ATOM | 3194 | CB | ASP | C | 178 | 13.358 | 6.384 | 3.764 | 1.00 | 43.13 | C |
| ATOM | 3195 | CG | ASP | C | 178 | 12.444 | 6.337 | 2.566 | 1.00 | 43.69 | C |
| ATOM | 3196 | OD1 | ASP | C | 178 | 11.413 | 5.630 | 2.621 | 1.00 | 44.71 | O |
| ATOM | 3197 | OD2 | ASP | C | 178 | 12.765 | 7.013 | 1.564 | 1.00 | 43.73 | O |
| ATOM | 3198 | N | HIS | C | 179 | 14.322 | 6.056 | 6.826 | 1.00 | 42.67 | N |
| ATOM | 3199 | CA | HIS | C | 179 | 15.295 | 5.673 | 7.854 | 1.00 | 42.46 | C |
| ATOM | 3200 | C | HIS | C | 179 | 14.773 | 4.487 | 8.667 | 1.00 | 42.26 | C |
| ATOM | 3201 | O | HIS | C | 179 | 15.517 | 3.543 | 8.936 | 1.00 | 42.32 | O |
| ATOM | 3202 | CB | HIS | C | 179 | 15.661 | 6.852 | 8.768 | 1.00 | 42.38 | C |
| ATOM | 3203 | CG | HIS | C | 179 | 16.314 | 7.995 | 8.050 | 1.00 | 43.02 | C |
| ATOM | 3204 | ND1 | HIS | C | 179 | 17.196 | 7.817 | 7.004 | 1.00 | 43.40 | N |
| ATOM | 3205 | CD2 | HIS | C | 179 | 16.218 | 9.333 | 8.232 | 1.00 | 43.49 | C |
| ATOM | 3206 | CE1 | HIS | C | 179 | 17.607 | 8.994 | 6.566 | 1.00 | 43.07 | C |
| ATOM | 3207 | NE2 | HIS | C | 179 | 17.029 | 9.932 | 7.295 | 1.00 | 43.32 | N |
| ATOM | 3208 | N | ILE | C | 180 | 13.491 | 4.516 | 9.026 | 1.00 | 41.78 | N |
| ATOM | 3209 | CA | ILE | C | 180 | 12.904 | 3.397 | 9.750 | 1.00 | 41.58 | C |
| ATOM | 3210 | C | ILE | C | 180 | 12.858 | 2.167 | 8.847 | 1.00 | 41.39 | C |
| ATOM | 3211 | O | ILE | C | 180 | 13.180 | 1.058 | 9.282 | 1.00 | 41.33 | O |
| ATOM | 3212 | CB | ILE | C | 180 | 11.501 | 3.729 | 10.329 | 1.00 | 41.74 | C |
| ATOM | 3213 | CG1 | ILE | C | 180 | 11.594 | 4.882 | 11.331 | 1.00 | 41.63 | C |
| ATOM | 3214 | CG2 | ILE | C | 180 | 10.890 | 2.510 | 11.022 | 1.00 | 41.30 | C |
| ATOM | 3215 | CD1 | ILE | C | 180 | 10.282 | 5.622 | 11.551 | 1.00 | 42.12 | C |
| ATOM | 3216 | N | ARG | C | 181 | 12.485 | 2.372 | 7.585 | 1.00 | 41.16 | N |
| ATOM | 3217 | CA | ARG | C | 181 | 12.361 | 1.267 | 6.634 | 1.00 | 40.86 | C |
| ATOM | 3218 | C | ARG | C | 181 | 13.702 | 0.560 | 6.443 | 1.00 | 40.80 | C |
| ATOM | 3219 | O | ARG | C | 181 | 13.751 | −0.663 | 6.338 | 1.00 | 40.68 | O |
| ATOM | 3220 | CB | ARG | C | 181 | 11.793 | 1.753 | 5.299 | 1.00 | 40.71 | C |
| ATOM | 3221 | CG | ARG | C | 181 | 10.309 | 2.107 | 5.343 | 1.00 | 40.81 | C |
| ATOM | 3222 | CD | ARG | C | 181 | 9.755 | 2.554 | 3.979 | 1.00 | 40.92 | C |
| ATOM | 3223 | NE | ARG | C | 181 | 8.398 | 3.095 | 4.106 | 1.00 | 41.07 | N |
| ATOM | 3224 | CZ | ARG | C | 181 | 8.060 | 4.375 | 3.936 | 1.00 | 41.08 | C |
| ATOM | 3225 | NH1 | ARG | C | 181 | 8.961 | 5.289 | 3.600 | 1.00 | 41.32 | N |
| ATOM | 3226 | NH2 | ARG | C | 181 | 6.800 | 4.746 | 4.090 | 1.00 | 41.18 | N |
| ATOM | 3227 | N | ALA | C | 182 | 14.783 | 1.340 | 6.441 | 1.00 | 40.79 | N |
| ATOM | 3228 | CA | ALA | C | 182 | 16.139 | 0.827 | 6.249 | 1.00 | 40.67 | C |
| ATOM | 3229 | C | ALA | C | 182 | 16.665 | −0.058 | 7.391 | 1.00 | 40.86 | C |
| ATOM | 3230 | O | ALA | C | 182 | 17.589 | −0.849 | 7.182 | 1.00 | 40.88 | O |
| ATOM | 3231 | CB | ALA | C | 182 | 17.095 | 1.976 | 5.996 | 1.00 | 40.59 | C |
| ATOM | 3232 | N | LEU | C | 183 | 16.082 | 0.074 | 8.584 | 1.00 | 40.90 | N |
| ATOM | 3233 | CA | LEU | C | 183 | 16.531 | −0.676 | 9.766 | 1.00 | 40.82 | C |
| ATOM | 3234 | C | LEU | C | 183 | 16.392 | −2.193 | 9.622 | 1.00 | 40.97 | C |
| ATOM | 3235 | O | LEU | C | 183 | 15.541 | −2.669 | 8.874 | 1.00 | 41.04 | O |
| ATOM | 3236 | CB | LEU | C | 183 | 15.774 | −0.209 | 11.014 | 1.00 | 40.58 | C |
| ATOM | 3237 | CG | LEU | C | 183 | 15.968 | 1.246 | 11.457 | 1.00 | 40.40 | C |
| ATOM | 3238 | CD1 | LEU | C | 183 | 15.016 | 1.593 | 12.601 | 1.00 | 40.14 | C |
| ATOM | 3239 | CD2 | LEU | C | 183 | 17.414 | 1.548 | 11.850 | 1.00 | 39.15 | C |
| ATOM | 3240 | N | ASP | C | 184 | 17.253 | −2.941 | 10.320 | 1.00 | 41.04 | N |
| ATOM | 3241 | CA | ASP | C | 184 | 17.039 | −4.372 | 10.538 | 1.00 | 41.12 | C |
| ATOM | 3242 | C | ASP | C | 184 | 16.374 | −4.545 | 11.897 | 1.00 | 41.22 | C |
| ATOM | 3243 | O | ASP | C | 184 | 17.025 | −4.424 | 12.941 | 1.00 | 41.19 | O |
| ATOM | 3244 | CB | ASP | C | 184 | 18.357 | −5.154 | 10.476 | 1.00 | 41.22 | C |
| ATOM | 3245 | CG | ASP | C | 184 | 18.164 | −6.681 | 10.594 | 1.00 | 41.66 | C |
| ATOM | 3246 | OD1 | ASP | C | 184 | 17.027 | −7.196 | 10.459 | 1.00 | 40.91 | O |
| ATOM | 3247 | OD2 | ASP | C | 184 | 19.179 | −7.375 | 10.818 | 1.00 | 42.44 | O |
| ATOM | 3248 | N | ARG | C | 185 | 15.070 | −4.815 | 11.874 | 1.00 | 41.38 | N |
| ATOM | 3249 | CA | ARG | C | 185 | 14.277 | −4.976 | 13.100 | 1.00 | 41.18 | C |
| ATOM | 3250 | C | ARG | C | 185 | 14.264 | −6.408 | 13.657 | 1.00 | 41.44 | C |
| ATOM | 3251 | O | ARG | C | 185 | 13.761 | −6.632 | 14.756 | 1.00 | 41.62 | O |
| ATOM | 3252 | CB | ARG | C | 185 | 12.839 | −4.486 | 12.878 | 1.00 | 41.04 | C |
| ATOM | 3253 | CG | ARG | C | 185 | 11.937 | −5.468 | 12.145 | 1.00 | 40.69 | C |
| ATOM | 3254 | CD | ARG | C | 185 | 10.501 | −4.998 | 12.102 | 1.00 | 40.54 | C |
| ATOM | 3255 | NE | ARG | C | 185 | 9.622 | −6.052 | 11.615 | 1.00 | 39.77 | N |
| ATOM | 3256 | CZ | ARG | C | 185 | 9.298 | −6.239 | 10.335 | 1.00 | 40.02 | C |
| ATOM | 3257 | NH1 | ARG | C | 185 | 9.757 | −5.422 | 9.399 | 1.00 | 38.72 | N |
| ATOM | 3258 | NH2 | ARG | C | 185 | 8.497 | −7.244 | 9.991 | 1.00 | 39.79 | N |
| ATOM | 3259 | N | LEU | C | 186 | 14.808 | −7.363 | 12.906 | 1.00 | 41.61 | N |
| ATOM | 3260 | CA | LEU | C | 186 | 14.722 | −8.778 | 13.274 | 1.00 | 42.18 | C |
| ATOM | 3261 | C | LEU | C | 186 | 15.772 | −9.192 | 14.305 | 1.00 | 42.44 | C |
| ATOM | 3262 | O | LEU | C | 186 | 16.700 | −9.947 | 13.998 | 1.00 | 42.53 | O |
| ATOM | 3263 | CB | LEU | C | 186 | 14.812 | −9.667 | 12.030 | 1.00 | 42.19 | C |
| ATOM | 3264 | CG | LEU | C | 186 | 13.863 | −9.343 | 10.877 | 1.00 | 42.88 | C |
| ATOM | 3265 | CD1 | LEU | C | 186 | 14.153 | −10.250 | 9.698 | 1.00 | 43.10 | C |
| ATOM | 3266 | CD2 | LEU | C | 186 | 12.391 | −9.446 | 11.296 | 1.00 | 44.04 | C |
| ATOM | 3267 | N | GLN | C | 187 | 15.605 | −8.698 | 15.530 | 1.00 | 42.80 | N |
| ATOM | 3268 | CA | GLN | C | 187 | 16.556 | −8.929 | 16.624 | 1.00 | 43.06 | C |
| ATOM | 3269 | C | GLN | C | 187 | 15.960 | −8.565 | 17.995 | 1.00 | 43.49 | C |
| ATOM | 3270 | O | GLN | C | 187 | 14.858 | −8.026 | 18.087 | 1.00 | 43.26 | O |
| ATOM | 3271 | CB | GLN | C | 187 | 17.851 | −8.135 | 16.392 | 1.00 | 42.93 | C |
| ATOM | 3272 | CG | GLN | C | 187 | 17.664 | −6.618 | 16.364 | 1.00 | 42.50 | C |
| ATOM | 3273 | CD | GLN | C | 187 | 18.887 | −5.890 | 15.854 | 1.00 | 42.42 | C |
| ATOM | 3274 | OE1 | GLN | C | 187 | 19.947 | −5.928 | 16.474 | 1.00 | 41.84 | O |
| ATOM | 3275 | NE2 | GLN | C | 187 | 18.746 | −5.218 | 14.718 | 1.00 | 41.26 | N |
| ATOM | 3276 | N | GLU | C | 188 | 16.702 | −8.886 | 19.050 | 1.00 | 44.01 | N |
| ATOM | 3277 | CA | GLU | C | 188 | 16.407 | −8.395 | 20.384 | 1.00 | 44.66 | C |
| ATOM | 3278 | C | GLU | C | 188 | 16.596 | −6.873 | 20.407 | 1.00 | 45.16 | C |
| ATOM | 3279 | O | GLU | C | 188 | 17.571 | −6.355 | 19.844 | 1.00 | 45.25 | O |
| ATOM | 3280 | CB | GLU | C | 188 | 17.324 | −9.084 | 21.408 | 1.00 | 44.64 | C |
| ATOM | 3281 | CG | GLU | C | 188 | 17.387 | −8.434 | 22.794 | 1.00 | 44.73 | C |
| ATOM | 3282 | CD | GLU | C | 188 | 16.090 | −8.538 | 23.581 | 1.00 | 44.88 | C |
| ATOM | 3283 | OE1 | GLU | C | 188 | 15.213 | −9.349 | 23.209 | 1.00 | 44.17 | O |
| ATOM | 3284 | OE2 | GLU | C | 188 | 15.958 | −7.804 | 24.586 | 1.00 | 45.61 | O |
| ATOM | 3285 | N | VAL | C | 189 | 15.654 | −6.170 | 21.039 | 1.00 | 45.59 | N |
| ATOM | 3286 | CA | VAL | C | 189 | 15.710 | −4.712 | 21.169 | 1.00 | 46.14 | C |
| ATOM | 3287 | C | VAL | C | 189 | 17.077 | −4.273 | 21.697 | 1.00 | 46.47 | C |
| ATOM | 3288 | O | VAL | C | 189 | 17.465 | −4.673 | 22.795 | 1.00 | 46.52 | O |
| ATOM | 3289 | CB | VAL | C | 189 | 14.629 | −4.180 | 22.136 | 1.00 | 46.25 | C |
| ATOM | 3290 | CG1 | VAL | C | 189 | 14.561 | −2.659 | 22.065 | 1.00 | 46.49 | C |
| ATOM | 3291 | CG2 | VAL | C | 189 | 13.260 | −4.803 | 21.838 | 1.00 | 46.36 | C |
| ATOM | 3292 | N | PRO | C | 190 | 17.824 | −3.475 | 20.907 | 1.00 | 46.80 | N |
| ATOM | 3293 | CA | PRO | C | 190 | 19.107 | −2.956 | 21.389 | 1.00 | 47.08 | C |
| ATOM | 3294 | C | PRO | C | 190 | 18.957 | −2.007 | 22.584 | 1.00 | 47.40 | C |
| ATOM | 3295 | O | PRO | C | 190 | 17.856 | −1.503 | 22.857 | 1.00 | 47.17 | O |
| ATOM | 3296 | CB | PRO | C | 190 | 19.667 | −2.203 | 20.173 | 1.00 | 47.01 | C |
| ATOM | 3297 | CG | PRO | C | 190 | 18.922 | −2.743 | 18.998 | 1.00 | 46.80 | C |
| ATOM | 3298 | CD | PRO | C | 190 | 17.550 | −3.040 | 19.526 | 1.00 | 46.85 | C |
| ATOM | 3299 | N | HIS | C | 191 | 20.061 | −1.797 | 23.298 | 1.00 | 47.73 | N |
| ATOM | 3300 | CA | HIS | C | 191 | 20.104 | −0.852 | 24.411 | 1.00 | 48.06 | C |
| ATOM | 3301 | C | HIS | C | 191 | 20.299 | 0.569 | 23.889 | 1.00 | 47.66 | C |
| ATOM | 3302 | O | HIS | C | 191 | 19.882 | 1.535 | 24.519 | 1.00 | 47.77 | O |
| ATOM | 3303 | CB | HIS | C | 191 | 21.232 | −1.206 | 25.388 | 1.00 | 48.41 | C |
| ATOM | 3304 | CG | HIS | C | 191 | 21.065 | −2.533 | 26.069 | 1.00 | 50.04 | C |
| ATOM | 3305 | ND1 | HIS | C | 191 | 19.966 | −2.843 | 26.846 | 1.00 | 51.52 | N |
| ATOM | 3306 | CD2 | HIS | C | 191 | 21.873 | −3.620 | 26.109 | 1.00 | 50.75 | C |
| ATOM | 3307 | CE1 | HIS | C | 191 | 20.100 | −4.068 | 27.326 | 1.00 | 51.55 | C |
| ATOM | 3308 | NE2 | HIS | C | 191 | 21.249 | −4.560 | 26.894 | 1.00 | 51.68 | N |
| ATOM | 3309 | N | GLU | C | 192 | 20.934 | 0.676 | 22.727 | 1.00 | 47.47 | N |
| ATOM | 3310 | CA | GLU | C | 192 | 21.276 | 1.959 | 22.115 | 1.00 | 47.17 | C |
| ATOM | 3311 | C | GLU | C | 192 | 20.715 | 2.047 | 20.697 | 1.00 | 46.44 | C |
| ATOM | 3312 | O | GLU | C | 192 | 20.396 | 1.032 | 20.080 | 1.00 | 46.14 | O |
| ATOM | 3313 | CB | GLU | C | 192 | 22.803 | 2.141 | 22.072 | 1.00 | 47.30 | C |
| ATOM | 3314 | CG | GLU | C | 192 | 23.470 | 2.400 | 23.425 | 1.00 | 49.10 | C |
| ATOM | 3315 | CD | GLU | C | 192 | 24.341 | 1.238 | 23.934 | 1.00 | 51.57 | C |
| ATOM | 3316 | OE1 | GLU | C | 192 | 25.563 | 1.455 | 24.125 | 1.00 | 52.32 | O |
| ATOM | 3317 | OE2 | GLU | C | 192 | 23.818 | 0.120 | 24.156 | 1.00 | 51.84 | O |
| ATOM | 3318 | N | GLY | C | 193 | 20.585 | 3.268 | 20.196 | 1.00 | 45.84 | N |
| ATOM | 3319 | CA | GLY | C | 193 | 20.318 | 3.489 | 18.784 | 1.00 | 45.53 | C |
| ATOM | 3320 | C | GLY | C | 193 | 18.867 | 3.596 | 18.356 | 1.00 | 45.41 | C |
| ATOM | 3321 | O | GLY | C | 193 | 17.955 | 3.459 | 19.177 | 1.00 | 45.60 | O |
| ATOM | 3322 | N | PRO | C | 194 | 18.649 | 3.821 | 17.049 | 1.00 | 45.20 | N |
| ATOM | 3323 | CA | PRO | C | 194 | 17.356 | 4.096 | 16.410 | 1.00 | 45.09 | C |
| ATOM | 3324 | C | PRO | C | 194 | 16.192 | 3.176 | 16.824 | 1.00 | 45.03 | C |
| ATOM | 3325 | O | PRO | C | 194 | 15.090 | 3.665 | 17.098 | 1.00 | 45.18 | O |
| ATOM | 3326 | CB | PRO | C | 194 | 17.664 | 3.936 | 14.909 | 1.00 | 44.97 | C |
| ATOM | 3327 | CG | PRO | C | 194 | 19.051 | 3.353 | 14.830 | 1.00 | 44.93 | C |
| ATOM | 3328 | CD | PRO | C | 194 | 19.740 | 3.807 | 16.060 | 1.00 | 45.04 | C |
| ATOM | 3329 | N | MET | C | 195 | 16.426 | 1.868 | 16.857 | 1.00 | 44.78 | N |
| ATOM | 3330 | CA | MET | C | 195 | 15.360 | 0.916 | 17.129 | 1.00 | 44.54 | C |
| ATOM | 3331 | C | MET | C | 195 | 14.899 | 1.045 | 18.573 | 1.00 | 44.68 | C |
| ATOM | 3332 | O | MET | C | 195 | 13.701 | 1.103 | 18.852 | 1.00 | 44.69 | O |
| ATOM | 3333 | CB | MET | C | 195 | 15.833 | −0.504 | 16.824 | 1.00 | 44.62 | C |
| ATOM | 3334 | CG | MET | C | 195 | 14.808 | −1.586 | 17.088 | 1.00 | 44.67 | C |
| ATOM | 3335 | SD | MET | C | 195 | 15.315 | −3.177 | 16.415 | 1.00 | 44.28 | 5 |
| ATOM | 3336 | CE | MET | C | 195 | 14.086 | −4.248 | 17.154 | 1.00 | 44.92 | C |
| ATOM | 3337 | N | CYS | C | 196 | 15.865 | 1.101 | 19.484 | 1.00 | 44.84 | N |
| ATOM | 3338 | CA | CYS | C | 196 | 15.596 | 1.275 | 20.902 | 1.00 | 44.61 | C |
| ATOM | 3339 | C | CYS | C | 196 | 14.736 | 2.503 | 21.120 | 1.00 | 44.50 | C |
| ATOM | 3340 | O | CYS | C | 196 | 13.724 | 2.435 | 21.822 | 1.00 | 44.61 | O |
| ATOM | 3341 | CB | CYS | C | 196 | 16.910 | 1.414 | 21.672 | 1.00 | 44.80 | C |
| ATOM | 3342 | SG | CYS | C | 196 | 16.771 | 2.122 | 23.330 | 1.00 | 45.09 | 5 |
| ATOM | 3343 | N | ASP | C | 197 | 15.134 | 3.614 | 20.496 | 1.00 | 44.22 | N |
| ATOM | 3344 | CA | ASP | C | 197 | 14.477 | 4.909 | 20.710 | 1.00 | 44.03 | C |
| ATOM | 3345 | C | ASP | C | 197 | 12.988 | 4.863 | 20.390 | 1.00 | 43.95 | C |
| ATOM | 3346 | O | ASP | C | 197 | 12.178 | 5.391 | 21.157 | 1.00 | 44.11 | O |
| ATOM | 3347 | CB | ASP | C | 197 | 15.147 | 6.029 | 19.904 | 1.00 | 43.91 | C |
| ATOM | 3348 | CG | ASP | C | 197 | 16.606 | 6.258 | 20.290 | 1.00 | 44.09 | C |
| ATOM | 3349 | OD1 | ASP | C | 197 | 17.043 | 5.820 | 21.377 | 1.00 | 43.48 | O |
| ATOM | 3350 | OD2 | ASP | C | 197 | 17.322 | 6.899 | 19.493 | 1.00 | 44.20 | O |
| ATOM | 3351 | N | LEU | C | 198 | 12.635 | 4.221 | 19.272 | 1.00 | 43.59 | N |
| ATOM | 3352 | CA | LEU | C | 198 | 11.243 | 4.148 | 18.813 | 1.00 | 43.18 | C |
| ATOM | 3353 | C | LEU | C | 198 | 10.366 | 3.434 | 19.814 | 1.00 | 42.85 | C |
| ATOM | 3354 | O | LEU | C | 198 | 9.155 | 3.619 | 19.829 | 1.00 | 42.59 | O |
| ATOM | 3355 | CB | LEU | C | 198 | 11.144 | 3.427 | 17.468 | 1.00 | 43.20 | C |
| ATOM | 3356 | CG | LEU | C | 198 | 11.873 | 4.024 | 16.263 | 1.00 | 43.81 | C |
| ATOM | 3357 | CD1 | LEU | C | 198 | 12.212 | 2.918 | 15.263 | 1.00 | 44.30 | C |
| ATOM | 3358 | CD2 | LEU | C | 198 | 11.070 | 5.152 | 15.607 | 1.00 | 43.35 | C |
| ATOM | 3359 | N | LEU | C | 199 | 10.990 | 2.614 | 20.649 | 1.00 | 42.87 | N |
| ATOM | 3360 | CA | LEU | C | 199 | 10.253 | 1.801 | 21.602 | 1.00 | 43.09 | C |
| ATOM | 3361 | C | LEU | C | 199 | 10.311 | 2.358 | 23.014 | 1.00 | 43.20 | C |
| ATOM | 3362 | O | LEU | C | 199 | 9.510 | 1.973 | 23.864 | 1.00 | 43.13 | O |
| ATOM | 3363 | CB | LEU | C | 199 | 10.759 | 0.353 | 21.574 | 1.00 | 42.93 | C |
| ATOM | 3364 | CG | LEU | C | 199 | 10.665 | −0.390 | 20.235 | 1.00 | 42.87 | C |
| ATOM | 3365 | CD1 | LEU | C | 199 | 10.846 | −1.891 | 20.438 | 1.00 | 43.22 | C |
| ATOM | 3366 | CD2 | LEU | C | 199 | 9.347 | −0.100 | 19.525 | 1.00 | 42.34 | C |
| ATOM | 3367 | N | TRP | C | 200 | 11.236 | 3.292 | 23.235 | 1.00 | 43.50 | N |
| ATOM | 3368 | CA | TRP | C | 200 | 11.588 | 3.748 | 24.575 | 1.00 | 43.72 | C |
| ATOM | 3369 | C | TRP | C | 200 | 11.480 | 5.248 | 24.829 | 1.00 | 43.99 | C |
| ATOM | 3370 | O | TRP | C | 200 | 11.359 | 5.654 | 25.981 | 1.00 | 44.54 | O |
| ATOM | 3371 | CB | TRP | C | 200 | 13.011 | 3.307 | 24.908 | 1.00 | 43.42 | C |
| ATOM | 3372 | CG | TRP | C | 200 | 13.138 | 1.895 | 25.398 | 1.00 | 42.97 | C |
| ATOM | 3373 | CD1 | TRP | C | 200 | 13.474 | 0.801 | 24.662 | 1.00 | 42.28 | C |
| ATOM | 3374 | CD2 | TRP | C | 200 | 12.966 | 1.436 | 26.743 | 1.00 | 42.67 | C |
| ATOM | 3375 | NE1 | TRP | C | 200 | 13.520 | −0.313 | 25.460 | 1.00 | 41.97 | N |
| ATOM | 3376 | CE2 | TRP | C | 200 | 13.209 | 0.049 | 26.744 | 1.00 | 42.99 | C |
| ATOM | 3377 | CE3 | TRP | C | 200 | 12.623 | 2.064 | 27.949 | 1.00 | 42.18 | C |
| ATOM | 3378 | CZ2 | TRP | C | 200 | 13.116 | −0.726 | 27.907 | 1.00 | 43.25 | C |
| ATOM | 3379 | CZ3 | TRP | C | 200 | 12.533 | 1.299 | 29.097 | 1.00 | 42.16 | C |
| ATOM | 3380 | CH2 | TRP | C | 200 | 12.780 | −0.081 | 29.070 | 1.00 | 42.79 | C |
| ATOM | 3381 | N | SER | C | 201 | 11.556 | 6.063 | 23.778 | 1.00 | 44.14 | N |
| ATOM | 3382 | CA | SER | C | 201 | 11.524 | 7.533 | 23.903 | 1.00 | 44.15 | C |
| ATOM | 3383 | C | SER | C | 201 | 10.205 | 8.103 | 24.439 | 1.00 | 44.27 | C |
| ATOM | 3384 | O | SER | C | 201 | 9.151 | 7.493 | 24.293 | 1.00 | 44.13 | O |
| ATOM | 3385 | CB | SER | C | 201 | 11.821 | 8.181 | 22.554 | 1.00 | 44.07 | C |
| ATOM | 3386 | OG | SER | C | 201 | 13.179 | 8.031 | 22.211 | 1.00 | 44.55 | O |
| ATOM | 3387 | N | ASP | C | 202 | 10.275 | 9.287 | 25.043 | 1.00 | 44.62 | N |
| ATOM | 3388 | CA | ASP | C | 202 | 9.085 | 9.968 | 25.558 | 1.00 | 45.06 | C |
| ATOM | 3389 | C | ASP | C | 202 | 8.970 | 11.407 | 25.051 | 1.00 | 45.35 | C |
| ATOM | 3390 | O | ASP | C | 202 | 9.987 | 12.096 | 24.914 | 1.00 | 45.28 | O |
| ATOM | 3391 | CB | ASP | C | 202 | 9.097 | 9.977 | 27.084 | 1.00 | 44.93 | C |
| ATOM | 3392 | CG | ASP | C | 202 | 8.832 | 8.623 | 27.667 | 1.00 | 45.81 | C |
| ATOM | 3393 | OD1 | ASP | C | 202 | 7.719 | 8.100 | 27.468 | 1.00 | 47.89 | O |
| ATOM | 3394 | OD2 | ASP | C | 202 | 9.732 | 8.072 | 28.331 | 1.00 | 47.59 | O |
| ATOM | 3395 | N | PRO | C | 203 | 7.730 | 11.869 | 24.778 | 1.00 | 45.50 | N |
| ATOM | 3396 | CA | PRO | C | 203 | 7.499 | 13.284 | 24.538 | 1.00 | 45.71 | C |
| ATOM | 3397 | C | PRO | C | 203 | 7.692 | 14.054 | 25.843 | 1.00 | 46.07 | C |
| ATOM | 3398 | O | PRO | C | 203 | 7.526 | 13.496 | 26.938 | 1.00 | 45.58 | O |
| ATOM | 3399 | CB | PRO | C | 203 | 6.039 | 13.334 | 24.084 | 1.00 | 45.53 | C |
| ATOM | 3400 | CG | PRO | C | 203 | 5.418 | 12.163 | 24.718 | 1.00 | 45.55 | C |
| ATOM | 3401 | CD | PRO | C | 203 | 6.480 | 11.098 | 24.688 | 1.00 | 45.61 | C |
| ATOM | 3402 | N | ASP | C | 204 | 8.007 | 15.336 | 25.704 | 1.00 | 46.60 | N |
| ATOM | 3403 | CA | ASP | C | 204 | 8.689 | 16.081 | 26.736 | 1.00 | 47.36 | C |
| ATOM | 3404 | C | ASP | C | 204 | 8.348 | 17.560 | 26.585 | 1.00 | 47.90 | C |
| ATOM | 3405 | O | ASP | C | 204 | 8.080 | 18.035 | 25.477 | 1.00 | 48.10 | O |
| ATOM | 3406 | CB | ASP | C | 204 | 10.183 | 15.846 | 26.529 | 1.00 | 47.60 | C |
| ATOM | 3407 | CG | ASP | C | 204 | 11.040 | 16.484 | 27.576 | 1.00 | 48.08 | C |
| ATOM | 3408 | OD1 | ASP | C | 204 | 11.064 | 15.967 | 28.717 | 1.00 | 49.49 | O |
| ATOM | 3409 | OD2 | ASP | C | 204 | 11.725 | 17.473 | 27.237 | 1.00 | 47.75 | O |
| ATOM | 3410 | N | ASP | C | 205 | 8.365 | 18.284 | 27.699 | 1.00 | 48.42 | N |
| ATOM | 3411 | CA | ASP | C | 205 | 7.975 | 19.698 | 27.731 | 1.00 | 48.81 | C |
| ATOM | 3412 | C | ASP | C | 205 | 9.156 | 20.671 | 27.633 | 1.00 | 48.71 | C |
| ATOM | 3413 | O | ASP | C | 205 | 8.968 | 21.887 | 27.737 | 1.00 | 48.66 | O |
| ATOM | 3414 | CB | ASP | C | 205 | 7.191 | 19.981 | 29.011 | 1.00 | 49.10 | C |
| ATOM | 3415 | CG | ASP | C | 205 | 7.926 | 19.499 | 30.247 | 1.00 | 50.41 | C |
| ATOM | 3416 | OD1 | ASP | C | 205 | 8.070 | 18.260 | 30.408 | 1.00 | 52.17 | O |
| ATOM | 3417 | OD2 | ASP | C | 205 | 8.374 | 20.355 | 31.042 | 1.00 | 50.98 | O |
| ATOM | 3418 | N | ARG | C | 206 | 10.367 | 20.148 | 27.455 | 1.00 | 48.70 | N |
| ATOM | 3419 | CA | ARG | C | 206 | 11.532 | 21.003 | 27.200 | 1.00 | 48.69 | C |
| ATOM | 3420 | C | ARG | C | 206 | 12.075 | 20.728 | 25.807 | 1.00 | 48.36 | C |
| ATOM | 3421 | O | ARG | C | 206 | 12.706 | 19.699 | 25.584 | 1.00 | 48.77 | O |
| ATOM | 3422 | CB | ARG | C | 206 | 12.650 | 20.800 | 28.234 | 1.00 | 48.75 | C |
| ATOM | 3423 | CG | ARG | C | 206 | 12.211 | 20.515 | 29.658 | 1.00 | 49.47 | C |
| ATOM | 3424 | CD | ARG | C | 206 | 12.402 | 19.037 | 29.986 | 1.00 | 51.17 | C |
| ATOM | 3425 | NE | ARG | C | 206 | 13.812 | 18.641 | 29.970 | 1.00 | 52.15 | N |
| ATOM | 3426 | CZ | ARG | C | 206 | 14.253 | 17.398 | 29.771 | 1.00 | 53.00 | C |
| ATOM | 3427 | NH1 | ARG | C | 206 | 13.406 | 16.398 | 29.560 | 1.00 | 53.27 | N |
| ATOM | 3428 | NH2 | ARG | C | 206 | 15.556 | 17.151 | 29.778 | 1.00 | 53.11 | N |
| ATOM | 3429 | N | GLY | C | 207 | 11.831 | 21.649 | 24.879 | 1.00 | 47.90 | N |
| ATOM | 3430 | CA | GLY | C | 207 | 12.276 | 21.516 | 23.485 | 1.00 | 47.36 | C |
| ATOM | 3431 | C | GLY | C | 207 | 13.649 | 20.896 | 23.283 | 1.00 | 46.80 | C |
| ATOM | 3432 | O | GLY | C | 207 | 14.524 | 21.007 | 24.137 | 1.00 | 46.77 | O |
| ATOM | 3433 | N | GLY | C | 208 | 13.835 | 20.231 | 22.151 | 1.00 | 46.37 | N |
| ATOM | 3434 | CA | GLY | C | 208 | 15.097 | 19.570 | 21.857 | 1.00 | 45.97 | C |
| ATOM | 3435 | C | GLY | C | 208 | 15.184 | 18.168 | 22.428 | 1.00 | 45.69 | C |
| ATOM | 3436 | O | GLY | C | 208 | 14.192 | 17.610 | 22.890 | 1.00 | 45.53 | O |
| ATOM | 3437 | N | TRP | C | 209 | 16.383 | 17.596 | 22.381 | 1.00 | 45.66 | N |
| ATOM | 3438 | CA | TRP | C | 209 | 16.623 | 16.237 | 22.868 | 1.00 | 45.59 | C |
| ATOM | 3439 | C | TRP | C | 209 | 17.138 | 16.225 | 24.308 | 1.00 | 45.37 | C |
| ATOM | 3440 | O | TRP | C | 209 | 17.914 | 17.101 | 24.706 | 1.00 | 45.52 | O |
| ATOM | 3441 | CB | TRP | C | 209 | 17.622 | 15.517 | 21.958 | 1.00 | 45.58 | C |
| ATOM | 3442 | CG | TRP | C | 209 | 17.134 | 15.301 | 20.542 | 1.00 | 45.49 | C |
| ATOM | 3443 | CD1 | TRP | C | 209 | 17.551 | 15.963 | 19.422 | 1.00 | 45.08 | C |
| ATOM | 3444 | CD2 | TRP | C | 209 | 16.152 | 14.351 | 20.105 | 1.00 | 44.87 | C |
| ATOM | 3445 | NE1 | TRP | C | 209 | 16.888 | 15.486 | 18.316 | 1.00 | 44.96 | N |
| ATOM | 3446 | CE2 | TRP | C | 209 | 16.026 | 14.494 | 18.705 | 1.00 | 45.01 | C |
| ATOM | 3447 | CE3 | TRP | C | 209 | 15.367 | 13.393 | 20.759 | 1.00 | 44.80 | C |
| ATOM | 3448 | CZ2 | TRP | C | 209 | 15.139 | 13.720 | 17.947 | 1.00 | 45.41 | C |
| ATOM | 3449 | CZ3 | TRP | C | 209 | 14.488 | 12.618 | 20.002 | 1.00 | 45.18 | C |
| ATOM | 3450 | CH2 | TRP | C | 209 | 14.378 | 12.790 | 18.614 | 1.00 | 45.37 | C |
| ATOM | 3451 | N | GLY | C | 210 | 16.701 | 15.232 | 25.079 | 1.00 | 45.00 | N |
| ATOM | 3452 | CA | GLY | C | 210 | 17.187 | 15.032 | 26.444 | 1.00 | 44.46 | C |
| ATOM | 3453 | C | GLY | C | 210 | 17.506 | 13.577 | 26.751 | 1.00 | 44.20 | C |
| ATOM | 3454 | O | GLY | C | 210 | 16.865 | 12.666 | 26.227 | 1.00 | 44.20 | O |
| ATOM | 3455 | N | ILE | C | 211 | 18.513 | 13.364 | 27.595 | 1.00 | 44.03 | N |
| ATOM | 3456 | CA | ILE | C | 211 | 18.854 | 12.031 | 28.109 | 1.00 | 43.74 | C |
| ATOM | 3457 | C | ILE | C | 211 | 17.641 | 11.480 | 28.860 | 1.00 | 43.18 | C |
| ATOM | 3458 | O | ILE | C | 211 | 17.120 | 12.140 | 29.769 | 1.00 | 42.92 | O |
| ATOM | 3459 | CB | ILE | C | 211 | 20.073 | 12.087 | 29.082 | 1.00 | 43.93 | C |
| ATOM | 3460 | CG1 | ILE | C | 211 | 21.093 | 13.138 | 28.624 | 1.00 | 44.22 | C |
| ATOM | 3461 | CG2 | ILE | C | 211 | 20.710 | 10.697 | 29.248 | 1.00 | 43.83 | C |
| ATOM | 3462 | CD1 | ILE | C | 211 | 21.862 | 13.807 | 29.757 | 1.00 | 45.10 | C |
| ATOM | 3463 | N | SER | C | 212 | 17.199 | 10.281 | 28.478 | 1.00 | 42.58 | N |
| ATOM | 3464 | CA | SER | C | 212 | 15.981 | 9.689 | 29.048 | 1.00 | 42.11 | C |
| ATOM | 3465 | C | SER | C | 212 | 16.170 | 9.195 | 30.482 | 1.00 | 42.03 | C |
| ATOM | 3466 | O | SER | C | 212 | 16.993 | 8.307 | 30.731 | 1.00 | 42.04 | O |
| ATOM | 3467 | CB | SER | C | 212 | 15.467 | 8.551 | 28.174 | 1.00 | 41.95 | C |
| ATOM | 3468 | OG | SER | C | 212 | 14.422 | 7.864 | 28.827 | 1.00 | 41.09 | O |
| ATOM | 3469 | N | PRO | C | 213 | 15.417 | 9.777 | 31.438 | 1.00 | 41.83 | N |
| ATOM | 3470 | CA | PRO | C | 213 | 15.506 | 9.296 | 32.823 | 1.00 | 41.67 | C |
| ATOM | 3471 | C | PRO | C | 213 | 15.036 | 7.854 | 32.985 | 1.00 | 41.47 | C |
| ATOM | 3472 | O | PRO | C | 213 | 15.238 | 7.257 | 34.034 | 1.00 | 41.75 | O |
| ATOM | 3473 | CB | PRO | C | 213 | 14.604 | 10.260 | 33.603 | 1.00 | 41.67 | C |
| ATOM | 3474 | CG | PRO | C | 213 | 13.775 | 10.949 | 32.598 | 1.00 | 41.54 | C |
| ATOM | 3475 | CD | PRO | C | 213 | 14.499 | 10.921 | 31.299 | 1.00 | 41.61 | C |
| ATOM | 3476 | N | ARG | C | 214 | 14.448 | 7.300 | 31.932 | 1.00 | 41.36 | N |
| ATOM | 3477 | CA | ARG | C | 214 | 13.936 | 5.928 | 31.919 | 1.00 | 41.20 | C |
| ATOM | 3478 | C | ARG | C | 214 | 15.057 | 4.876 | 31.847 | 1.00 | 41.09 | C |
| ATOM | 3479 | O | ARG | C | 214 | 14.847 | 3.713 | 32.204 | 1.00 | 41.12 | O |
| ATOM | 3480 | CB | ARG | C | 214 | 12.986 | 5.762 | 30.728 | 1.00 | 41.08 | C |
| ATOM | 3481 | CG | ARG | C | 214 | 11.729 | 4.979 | 31.003 | 1.00 | 40.99 | C |
| ATOM | 3482 | CD | ARG | C | 214 | 10.882 | 4.849 | 29.735 | 1.00 | 41.20 | C |
| ATOM | 3483 | NE | ARG | C | 214 | 9.707 | 5.726 | 29.732 | 1.00 | 41.65 | N |
| ATOM | 3484 | CZ | ARG | C | 214 | 8.482 | 5.361 | 30.122 | 1.00 | 41.84 | C |
| ATOM | 3485 | NH1 | ARG | C | 214 | 8.233 | 4.129 | 30.561 | 1.00 | 41.01 | N |
| ATOM | 3486 | NH2 | ARG | C | 214 | 7.492 | 6.238 | 30.077 | 1.00 | 41.88 | N |
| ATOM | 3487 | N | GLY | C | 215 | 16.238 | 5.290 | 31.386 | 1.00 | 40.80 | N |
| ATOM | 3488 | CA | GLY | C | 215 | 17.365 | 4.383 | 31.166 | 1.00 | 40.38 | C |
| ATOM | 3489 | C | GLY | C | 215 | 17.734 | 4.212 | 29.696 | 1.00 | 40.35 | C |
| ATOM | 3490 | O | GLY | C | 215 | 18.906 | 4.057 | 29.363 | 1.00 | 40.30 | O |
| ATOM | 3491 | N | ALA | C | 216 | 16.731 | 4.238 | 28.819 | 1.00 | 40.16 | N |
| ATOM | 3492 | CA | ALA | C | 216 | 16.935 | 4.050 | 27.383 | 1.00 | 39.97 | C |
| ATOM | 3493 | C | ALA | C | 216 | 16.051 | 4.993 | 26.570 | 1.00 | 39.94 | C |
| ATOM | 3494 | O | ALA | C | 216 | 14.961 | 5.362 | 27.011 | 1.00 | 39.93 | O |
| ATOM | 3495 | CB | ALA | C | 216 | 16.657 | 2.603 | 26.989 | 1.00 | 39.93 | C |
| ATOM | 3496 | N | GLY | C | 217 | 16.522 | 5.363 | 25.380 | 1.00 | 39.66 | N |
| ATOM | 3497 | CA | GLY | C | 217 | 15.781 | 6.254 | 24.499 | 1.00 | 39.38 | C |
| ATOM | 3498 | C | GLY | C | 217 | 16.054 | 7.709 | 24.818 | 1.00 | 39.39 | C |
| ATOM | 3499 | O | GLY | C | 217 | 17.065 | 8.039 | 25.432 | 1.00 | 39.67 | O |
| ATOM | 3500 | N | TYR | C | 218 | 15.150 | 8.585 | 24.397 | 1.00 | 39.26 | N |
| ATOM | 3501 | CA | TYR | C | 218 | 15.356 | 10.021 | 24.516 | 1.00 | 39.13 | C |
| ATOM | 3502 | C | TYR | C | 218 | 14.061 | 10.729 | 24.838 | 1.00 | 39.08 | C |
| ATOM | 3503 | O | TYR | C | 218 | 12.996 | 10.351 | 24.344 | 1.00 | 39.36 | O |
| ATOM | 3504 | CB | TYR | C | 218 | 15.897 | 10.594 | 23.197 | 1.00 | 39.03 | C |
| ATOM | 3505 | CG | TYR | C | 218 | 17.315 | 10.192 | 22.865 | 1.00 | 39.02 | C |
| ATOM | 3506 | CD1 | TYR | C | 218 | 17.589 | 8.946 | 22.294 | 1.00 | 38.64 | C |
| ATOM | 3507 | CD2 | TYR | C | 218 | 18.383 | 11.057 | 23.116 | 1.00 | 38.10 | C |
| ATOM | 3508 | CE1 | TYR | C | 218 | 18.887 | 8.565 | 21.991 | 1.00 | 38.70 | C |
| ATOM | 3509 | CE2 | TYR | C | 218 | 19.693 | 10.684 | 22.813 | 1.00 | 37.90 | C |
| ATOM | 3510 | CZ | TYR | C | 218 | 19.937 | 9.438 | 22.249 | 1.00 | 38.54 | C |
| ATOM | 3511 | OH | TYR | C | 218 | 21.222 | 9.052 | 21.936 | 1.00 | 38.44 | O |
| ATOM | 3512 | N | THR | C | 219 | 14.141 | 11.767 | 25.656 | 1.00 | 38.85 | N |
| ATOM | 3513 | CA | THR | C | 219 | 13.023 | 12.681 | 25.741 | 1.00 | 38.75 | C |
| ATOM | 3514 | C | THR | C | 219 | 13.149 | 13.610 | 24.541 | 1.00 | 38.68 | C |
| ATOM | 3515 | O | THR | C | 219 | 14.263 | 13.979 | 24.162 | 1.00 | 38.90 | O |
| ATOM | 3516 | CB | THR | C | 219 | 12.976 | 13.459 | 27.073 | 1.00 | 38.71 | C |
| ATOM | 3517 | OG1 | THR | C | 219 | 14.064 | 14.391 | 27.141 | 1.00 | 38.92 | O |
| ATOM | 3518 | CG2 | THR | C | 219 | 13.026 | 12.495 | 28.255 | 1.00 | 38.11 | C |
| ATOM | 3519 | N | PHE | C | 220 | 12.017 | 13.950 | 23.929 | 1.00 | 38.48 | N |
| ATOM | 3520 | CA | PHE | C | 220 | 11.998 | 14.848 | 22.782 | 1.00 | 38.39 | C |
| ATOM | 3521 | C | PHE | C | 220 | 10.930 | 15.913 | 22.938 | 1.00 | 38.59 | C |
| ATOM | 3522 | O | PHE | C | 220 | 9.845 | 15.635 | 23.435 | 1.00 | 38.40 | O |
| ATOM | 3523 | CB | PHE | C | 220 | 11.818 | 14.074 | 21.468 | 1.00 | 38.46 | C |
| ATOM | 3524 | CG | PHE | C | 220 | 10.514 | 13.300 | 21.364 | 1.00 | 38.49 | C |
| ATOM | 3525 | CD1 | PHE | C | 220 | 9.376 | 13.891 | 20.814 | 1.00 | 38.06 | C |
| ATOM | 3526 | CD2 | PHE | C | 220 | 10.443 | 11.966 | 21.772 | 1.00 | 37.62 | C |
| ATOM | 3527 | CE1 | PHE | C | 220 | 8.188 | 13.178 | 20.706 | 1.00 | 39.12 | C |
| ATOM | 3528 | CE2 | PHE | C | 220 | 9.261 | 11.252 | 21.671 | 1.00 | 37.63 | C |
| ATOM | 3529 | CZ | PHE | C | 220 | 8.130 | 11.852 | 21.140 | 1.00 | 38.59 | C |
| ATOM | 3530 | N | GLY | C | 221 | 11.247 | 17.128 | 22.499 | 1.00 | 39.07 | N |
| ATOM | 3531 | CA | GLY | C | 221 | 10.360 | 18.277 | 22.662 | 1.00 | 39.65 | C |
| ATOM | 3532 | C | GLY | C | 221 | 9.475 | 18.557 | 21.463 | 1.00 | 40.10 | C |
| ATOM | 3533 | O | GLY | C | 221 | 9.574 | 17.875 | 20.438 | 1.00 | 40.24 | O |
| ATOM | 3534 | N | GLN | C | 222 | 8.618 | 19.574 | 21.600 | 1.00 | 40.42 | N |
| ATOM | 3535 | CA | GLN | C | 222 | 7.626 | 19.936 | 20.584 | 1.00 | 40.69 | C |
| ATOM | 3536 | C | GLN | C | 222 | 8.275 | 20.330 | 19.258 | 1.00 | 40.82 | C |
| ATOM | 3537 | O | GLN | C | 222 | 7.792 | 19.948 | 18.186 | 1.00 | 40.87 | O |
| ATOM | 3538 | CB | GLN | C | 222 | 6.702 | 21.050 | 21.095 | 1.00 | 40.50 | C |
| ATOM | 3539 | CG | GLN | C | 222 | 5.686 | 21.549 | 20.064 | 1.00 | 40.78 | C |
| ATOM | 3540 | CD | GLN | C | 222 | 4.584 | 22.414 | 20.658 | 1.00 | 41.17 | C |
| ATOM | 3541 | OE1 | GLN | C | 222 | 4.254 | 22.305 | 21.844 | 1.00 | 42.78 | O |
| ATOM | 3542 | NE2 | GLN | C | 222 | 3.995 | 23.270 | 19.827 | 1.00 | 40.07 | N |
| ATOM | 3543 | N | ASP | C | 223 | 9.371 | 21.084 | 19.350 | 1.00 | 41.00 | N |
| ATOM | 3544 | CA | ASP | C | 223 | 10.182 | 21.486 | 18.190 | 1.00 | 41.25 | C |
| ATOM | 3545 | C | ASP | C | 223 | 10.594 | 20.292 | 17.329 | 1.00 | 41.10 | C |
| ATOM | 3546 | O | ASP | C | 223 | 10.718 | 20.400 | 16.108 | 1.00 | 41.05 | O |
| ATOM | 3547 | CB | ASP | C | 223 | 11.435 | 22.251 | 18.645 | 1.00 | 41.29 | C |
| ATOM | 3548 | CG | ASP | C | 223 | 12.146 | 21.578 | 19.821 | 1.00 | 41.95 | C |
| ATOM | 3549 | OD1 | ASP | C | 223 | 11.692 | 20.501 | 20.277 | 1.00 | 42.94 | O |
| ATOM | 3550 | OD2 | ASP | C | 223 | 13.154 | 22.132 | 20.305 | 1.00 | 41.99 | O |
| ATOM | 3551 | N | ILE | C | 224 | 10.799 | 19.153 | 17.974 | 1.00 | 41.06 | N |
| ATOM | 3552 | CA | ILE | C | 224 | 11.186 | 17.949 | 17.258 | 1.00 | 41.25 | C |
| ATOM | 3553 | C | ILE | C | 224 | 9.977 | 17.294 | 16.591 | 1.00 | 41.27 | C |
| ATOM | 3554 | O | ILE | C | 224 | 10.045 | 16.916 | 15.421 | 1.00 | 41.22 | O |
| ATOM | 3555 | CB | ILE | C | 224 | 11.989 | 17.002 | 18.163 | 1.00 | 41.16 | C |
| ATOM | 3556 | CG1 | ILE | C | 224 | 13.421 | 17.514 | 18.243 | 1.00 | 40.81 | C |
| ATOM | 3557 | CG2 | ILE | C | 224 | 11.983 | 15.574 | 17.624 | 1.00 | 41.32 | C |
| ATOM | 3558 | CD1 | ILE | C | 224 | 14.116 | 17.114 | 19.472 | 1.00 | 42.33 | C |
| ATOM | 3559 | N | SER | C | 225 | 8.876 | 17.195 | 17.330 | 1.00 | 41.29 | N |
| ATOM | 3560 | CA | SER | C | 225 | 7.612 | 16.704 | 16.790 | 1.00 | 41.80 | C |
| ATOM | 3561 | C | SER | C | 225 | 7.189 | 17.432 | 15.509 | 1.00 | 42.02 | C |
| ATOM | 3562 | O | SER | C | 225 | 6.838 | 16.796 | 14.512 | 1.00 | 41.74 | O |
| ATOM | 3563 | CB | SER | C | 225 | 6.506 | 16.851 | 17.836 | 1.00 | 41.70 | C |
| ATOM | 3564 | OG | SER | C | 225 | 6.812 | 16.109 | 18.996 | 1.00 | 42.77 | O |
| ATOM | 3565 | N | GLU | C | 226 | 7.218 | 18.764 | 15.560 | 1.00 | 42.44 | N |
| ATOM | 3566 | CA | GLU | C | 226 | 6.781 | 19.619 | 14.462 | 1.00 | 42.80 | C |
| ATOM | 3567 | C | GLU | C | 226 | 7.687 | 19.465 | 13.246 | 1.00 | 42.89 | C |
| ATOM | 3568 | O | GLU | C | 226 | 7.219 | 19.420 | 12.100 | 1.00 | 42.90 | O |
| ATOM | 3569 | CB | GLU | C | 226 | 6.772 | 21.079 | 14.912 | 1.00 | 42.82 | C |
| ATOM | 3570 | CG | GLU | C | 226 | 5.730 | 21.407 | 15.980 | 1.00 | 43.32 | C |
| ATOM | 3571 | CD | GLU | C | 226 | 5.823 | 22.850 | 16.482 | 1.00 | 43.36 | C |
| ATOM | 3572 | OE1 | GLU | C | 226 | 4.946 | 23.237 | 17.286 | 1.00 | 43.83 | O |
| ATOM | 3573 | OE2 | GLU | C | 226 | 6.762 | 23.590 | 16.084 | 1.00 | 42.42 | O |
| ATOM | 3574 | N | THR | C | 227 | 8.987 | 19.391 | 13.508 | 1.00 | 42.98 | N |
| ATOM | 3575 | CA | THR | C | 227 | 9.968 | 19.159 | 12.469 | 1.00 | 43.11 | C |
| ATOM | 3576 | C | THR | C | 227 | 9.716 | 17.807 | 11.813 | 1.00 | 43.29 | C |
| ATOM | 3577 | O | THR | C | 227 | 9.628 | 17.720 | 10.581 | 1.00 | 43.54 | O |
| ATOM | 3578 | CB | THR | C | 227 | 11.389 | 19.226 | 13.041 | 1.00 | 43.13 | C |
| ATOM | 3579 | OG1 | THR | C | 227 | 11.663 | 20.571 | 13.446 | 1.00 | 43.06 | O |
| ATOM | 3580 | CG2 | THR | C | 227 | 12.422 | 18.794 | 12.009 | 1.00 | 43.23 | C |
| ATOM | 3581 | N | PHE | C | 228 | 9.578 | 16.766 | 12.638 | 1.00 | 43.21 | N |
| ATOM | 3582 | CA | PHE | C | 228 | 9.424 | 15.401 | 12.147 | 1.00 | 43.10 | C |
| ATOM | 3583 | C | PHE | C | 228 | 8.160 | 15.250 | 11.307 | 1.00 | 43.55 | C |
| ATOM | 3584 | O | PHE | C | 228 | 8.213 | 14.750 | 10.181 | 1.00 | 43.80 | O |
| ATOM | 3585 | CB | PHE | C | 228 | 9.441 | 14.398 | 13.298 | 1.00 | 42.68 | C |
| ATOM | 3586 | CG | PHE | C | 228 | 9.359 | 12.966 | 12.851 | 1.00 | 42.87 | C |
| ATOM | 3587 | CD1 | PHE | C | 228 | 10.504 | 12.275 | 12.472 | 1.00 | 42.91 | C |
| ATOM | 3588 | CD2 | PHE | C | 228 | 8.135 | 12.305 | 12.801 | 1.00 | 42.14 | C |
| ATOM | 3589 | CE1 | PHE | C | 228 | 10.431 | 10.947 | 12.049 | 1.00 | 42.34 | C |
| ATOM | 3590 | CE2 | PHE | C | 228 | 8.059 | 10.982 | 12.385 | 1.00 | 41.78 | C |
| ATOM | 3591 | CZ | PHE | C | 228 | 9.206 | 10.304 | 12.005 | 1.00 | 42.00 | C |
| ATOM | 3592 | N | ASN | C | 229 | 7.029 | 15.689 | 11.854 | 1.00 | 43.92 | N |
| ATOM | 3593 | CA | ASN | C | 229 | 5.757 | 15.640 | 11.143 | 1.00 | 44.09 | C |
| ATOM | 3594 | C | ASN | C | 229 | 5.792 | 16.367 | 9.812 | 1.00 | 44.00 | C |
| ATOM | 3595 | O | ASN | C | 229 | 5.375 | 15.814 | 8.796 | 1.00 | 43.99 | O |
| ATOM | 3596 | CB | ASN | C | 229 | 4.624 | 16.196 | 12.008 | 1.00 | 44.25 | C |
| ATOM | 3597 | CG | ASN | C | 229 | 4.183 | 15.230 | 13.073 | 1.00 | 44.76 | C |
| ATOM | 3598 | OD1 | ASN | C | 229 | 4.262 | 14.016 | 12.891 | 1.00 | 46.27 | O |
| ATOM | 3599 | ND2 | ASN | C | 229 | 3.707 | 15.758 | 14.193 | 1.00 | 45.54 | N |
| ATOM | 3600 | N | HIS | C | 230 | 6.296 | 17.599 | 9.818 | 1.00 | 44.01 | N |
| ATOM | 3601 | CA | HIS | C | 230 | 6.286 | 18.412 | 8.611 | 1.00 | 44.05 | C |
| ATOM | 3602 | C | HIS | C | 230 | 7.118 | 17.804 | 7.481 | 1.00 | 43.96 | C |
| ATOM | 3603 | O | HIS | C | 230 | 6.713 | 17.863 | 6.313 | 1.00 | 44.08 | O |
| ATOM | 3604 | CB | HIS | C | 230 | 6.718 | 19.851 | 8.879 | 1.00 | 43.95 | C |
| ATOM | 3605 | CG | HIS | C | 230 | 6.658 | 20.714 | 7.659 | 1.00 | 44.75 | C |
| ATOM | 3606 | ND1 | HIS | C | 230 | 7.787 | 21.154 | 7.000 | 1.00 | 45.31 | N |
| ATOM | 3607 | CD2 | HIS | C | 230 | 5.603 | 21.170 | 6.944 | 1.00 | 45.20 | C |
| ATOM | 3608 | CE1 | HIS | C | 230 | 7.431 | 21.868 | 5.948 | 1.00 | 45.21 | C |
| ATOM | 3609 | NE2 | HIS | C | 230 | 6.111 | 21.892 | 5.890 | 1.00 | 45.74 | N |
| ATOM | 3610 | N | ALA | C | 231 | 8.263 | 17.219 | 7.832 | 1.00 | 43.63 | N |
| ATOM | 3611 | CA | ALA | C | 231 | 9.164 | 16.626 | 6.843 | 1.00 | 43.51 | C |
| ATOM | 3612 | C | ALA | C | 231 | 8.617 | 15.333 | 6.227 | 1.00 | 43.42 | C |
| ATOM | 3613 | O | ALA | C | 231 | 8.893 | 15.027 | 5.068 | 1.00 | 43.55 | O |
| ATOM | 3614 | CB | ALA | C | 231 | 10.539 | 16.388 | 7.447 | 1.00 | 43.53 | C |
| ATOM | 3615 | N | ASN | C | 232 | 7.849 | 14.577 | 7.000 | 1.00 | 43.13 | N |
| ATOM | 3616 | CA | ASN | C | 232 | 7.293 | 13.311 | 6.519 | 1.00 | 42.88 | C |
| ATOM | 3617 | C | ASN | C | 232 | 5.842 | 13.429 | 6.048 | 1.00 | 42.80 | C |
| ATOM | 3618 | O | ASN | C | 232 | 5.187 | 12.423 | 5.775 | 1.00 | 42.96 | O |
| ATOM | 3619 | CB | ASN | C | 232 | 7.420 | 12.229 | 7.599 | 1.00 | 42.62 | C |
| ATOM | 3620 | CG | ASN | C | 232 | 8.861 | 11.951 | 7.975 | 1.00 | 42.04 | C |
| ATOM | 3621 | OD1 | ASN | C | 232 | 9.558 | 11.214 | 7.289 | 1.00 | 41.55 | O |
| ATOM | 3622 | ND2 | ASN | C | 232 | 9.309 | 12.536 | 9.075 | 1.00 | 41.40 | N |
| ATOM | 3623 | N | GLY | C | 233 | 5.346 | 14.662 | 5.961 | 1.00 | 42.71 | N |
| ATOM | 3624 | CA | GLY | C | 233 | 3.948 | 14.928 | 5.595 | 1.00 | 42.57 | C |
| ATOM | 3625 | C | GLY | C | 233 | 2.903 | 14.339 | 6.534 | 1.00 | 42.33 | C |
| ATOM | 3626 | O | GLY | C | 233 | 1.802 | 14.006 | 6.099 | 1.00 | 42.27 | O |
| ATOM | 3627 | N | LEU | C | 234 | 3.244 | 14.223 | 7.817 | 1.00 | 42.16 | N |
| ATOM | 3628 | CA | LEU | C | 234 | 2.375 | 13.592 | 8.814 | 1.00 | 42.16 | C |
| ATOM | 3629 | C | LEU | C | 234 | 1.487 | 14.596 | 9.533 | 1.00 | 42.02 | C |
| ATOM | 3630 | O | LEU | C | 234 | 1.911 | 15.718 | 9.794 | 1.00 | 42.03 | O |
| ATOM | 3631 | CB | LEU | C | 234 | 3.217 | 12.851 | 9.855 | 1.00 | 42.22 | C |
| ATOM | 3632 | CG | LEU | C | 234 | 4.107 | 11.691 | 9.405 | 1.00 | 42.37 | C |
| ATOM | 3633 | CD1 | LEU | C | 234 | 5.077 | 11.330 | 10.517 | 1.00 | 42.26 | C |
| ATOM | 3634 | CD2 | LEU | C | 234 | 3.274 | 10.481 | 8.993 | 1.00 | 41.13 | C |
| ATOM | 3635 | N | THR | C | 235 | 0.266 | 14.189 | 9.870 | 1.00 | 41.94 | N |
| ATOM | 3636 | CA | THR | C | 235 | −0.619 | 15.059 | 10.655 | 1.00 | 42.30 | C |
| ATOM | 3637 | C | THR | C | 235 | −0.586 | 14.745 | 12.158 | 1.00 | 42.20 | C |
| ATOM | 3638 | O | THR | C | 235 | −0.821 | 15.630 | 12.986 | 1.00 | 42.29 | O |
| ATOM | 3639 | CB | THR | C | 235 | −2.099 | 15.061 | 10.159 | 1.00 | 42.46 | C |
| ATOM | 3640 | OG1 | THR | C | 235 | −2.752 | 13.848 | 10.563 | 1.00 | 43.10 | O |
| ATOM | 3641 | CG2 | THR | C | 235 | −2.193 | 15.246 | 8.636 | 1.00 | 42.25 | C |
| ATOM | 3642 | N | LEU | C | 236 | −0.293 | 13.495 | 12.507 | 1.00 | 41.92 | N |
| ATOM | 3643 | CA | LEU | C | 236 | −0.251 | 13.106 | 13.909 | 1.00 | 41.69 | C |
| ATOM | 3644 | C | LEU | C | 236 | 0.742 | 11.987 | 14.216 | 1.00 | 41.73 | C |
| ATOM | 3645 | O | LEU | C | 236 | 0.915 | 11.055 | 13.425 | 1.00 | 41.88 | O |
| ATOM | 3646 | CB | LEU | C | 236 | −1.653 | 12.704 | 14.380 | 1.00 | 41.69 | C |
| ATOM | 3647 | CG | LEU | C | 236 | −1.847 | 12.268 | 15.837 | 1.00 | 41.29 | C |
| ATOM | 3648 | CD1 | LEU | C | 236 | −1.472 | 13.376 | 16.811 | 1.00 | 39.79 | C |
| ATOM | 3649 | CD2 | LEU | C | 236 | −3.282 | 11.828 | 16.039 | 1.00 | 41.50 | C |
| ATOM | 3650 | N | VAL | C | 237 | 1.383 | 12.087 | 15.377 | 1.00 | 41.63 | N |
| ATOM | 3651 | CA | VAL | C | 237 | 2.157 | 10.983 | 15.931 | 1.00 | 41.35 | C |
| ATOM | 3652 | C | VAL | C | 237 | 1.370 | 10.416 | 17.094 | 1.00 | 41.59 | C |
| ATOM | 3653 | O | VAL | C | 237 | 1.087 | 11.111 | 18.070 | 1.00 | 41.63 | O |
| ATOM | 3654 | CB | VAL | C | 237 | 3.582 | 11.405 | 16.392 | 1.00 | 41.40 | C |
| ATOM | 3655 | CG1 | VAL | C | 237 | 4.242 | 10.286 | 17.175 | 1.00 | 40.78 | C |
| ATOM | 3656 | CG2 | VAL | C | 237 | 4.455 | 11.795 | 15.191 | 1.00 | 40.51 | C |
| ATOM | 3657 | N | SER | C | 238 | 1.000 | 9.150 | 16.957 | 1.00 | 41.87 | N |
| ATOM | 3658 | CA | SER | C | 238 | 0.235 | 8.436 | 17.954 | 1.00 | 42.10 | C |
| ATOM | 3659 | C | SER | C | 238 | 1.120 | 7.365 | 18.564 | 1.00 | 42.27 | C |
| ATOM | 3660 | O | SER | C | 238 | 1.720 | 6.562 | 17.852 | 1.00 | 42.61 | O |
| ATOM | 3661 | CB | SER | C | 238 | −0.995 | 7.793 | 17.316 | 1.00 | 42.09 | C |
| ATOM | 3662 | OG | SER | C | 238 | −1.639 | 6.912 | 18.224 | 1.00 | 42.35 | O |
| ATOM | 3663 | N | ARG | C | 239 | 1.176 | 7.353 | 19.887 | 1.00 | 42.27 | N |
| ATOM | 3664 | CA | ARG | C | 239 | 2.088 | 6.495 | 20.615 | 1.00 | 42.26 | C |
| ATOM | 3665 | C | ARG | C | 239 | 1.520 | 6.123 | 21.992 | 1.00 | 42.69 | C |
| ATOM | 3666 | O | ARG | C | 239 | 0.484 | 6.655 | 22.429 | 1.00 | 42.70 | O |
| ATOM | 3667 | CB | ARG | C | 239 | 3.466 | 7.171 | 20.743 | 1.00 | 42.43 | C |
| ATOM | 3668 | CG | ARG | C | 239 | 3.445 | 8.656 | 21.190 | 1.00 | 41.53 | C |
| ATOM | 3669 | CD | ARG | C | 239 | 3.371 | 8.812 | 22.702 | 1.00 | 39.87 | C |
| ATOM | 3670 | NE | ARG | C | 239 | 4.573 | 8.299 | 23.361 | 1.00 | 39.98 | N |
| ATOM | 3671 | CZ | ARG | C | 239 | 4.825 | 8.394 | 24.666 | 1.00 | 40.07 | C |
| ATOM | 3672 | NH1 | ARG | C | 239 | 3.963 | 8.983 | 25.486 | 1.00 | 38.77 | N |
| ATOM | 3673 | NH2 | ARG | C | 239 | 5.944 | 7.888 | 25.161 | 1.00 | 40.18 | N |
| ATOM | 3674 | N | ALA | C | 240 | 2.212 | 5.208 | 22.668 | 1.00 | 42.60 | N |
| ATOM | 3675 | CA | ALA | C | 240 | 1.745 | 4.638 | 23.925 | 1.00 | 42.23 | C |
| ATOM | 3676 | C | ALA | C | 240 | 2.827 | 4.833 | 25.000 | 1.00 | 41.85 | C |
| ATOM | 3677 | O | ALA | C | 240 | 3.325 | 5.941 | 25.168 | 1.00 | 41.62 | O |
| ATOM | 3678 | CB | ALA | C | 240 | 1.412 | 3.174 | 23.715 | 1.00 | 42.18 | C |
| ATOM | 3679 | N | HIS | C | 241 | 3.168 | 3.778 | 25.738 | 1.00 | 41.47 | N |
| ATOM | 3680 | CA | HIS | C | 241 | 4.397 | 3.757 | 26.540 | 1.00 | 41.47 | C |
| ATOM | 3681 | C | HIS | C | 241 | 4.340 | 4.515 | 27.890 | 1.00 | 41.25 | C |
| ATOM | 3682 | O | HIS | C | 241 | 5.007 | 4.122 | 28.855 | 1.00 | 41.16 | O |
| ATOM | 3683 | CB | HIS | C | 241 | 5.582 | 4.227 | 25.662 | 1.00 | 41.43 | C |
| ATOM | 3684 | CG | HIS | C | 241 | 6.929 | 4.026 | 26.281 | 1.00 | 41.37 | C |
| ATOM | 3685 | ND1 | HIS | C | 241 | 7.362 | 2.802 | 26.747 | 1.00 | 40.37 | N |
| ATOM | 3686 | CD2 | HIS | C | 241 | 7.949 | 4.893 | 26.487 | 1.00 | 41.45 | C |
| ATOM | 3687 | CE1 | HIS | C | 241 | 8.587 | 2.926 | 27.223 | 1.00 | 41.05 | C |
| ATOM | 3688 | NE2 | HIS | C | 241 | 8.970 | 4.183 | 27.074 | 1.00 | 41.71 | N |
| ATOM | 3689 | N | GLN | C | 242 | 3.555 | 5.588 | 27.952 | 1.00 | 40.92 | N |
| ATOM | 3690 | CA | GLN | C | 242 | 3.423 | 6.381 | 29.177 | 1.00 | 40.87 | C |
| ATOM | 3691 | C | GLN | C | 242 | 2.045 | 6.245 | 29.810 | 1.00 | 41.03 | C |
| ATOM | 3692 | O | GLN | C | 242 | 1.030 | 6.469 | 29.138 | 1.00 | 41.04 | O |
| ATOM | 3693 | CB | GLN | C | 242 | 3.715 | 7.855 | 28.903 | 1.00 | 40.49 | C |
| ATOM | 3694 | CG | GLN | C | 242 | 5.020 | 8.320 | 29.501 | 1.00 | 40.82 | C |
| ATOM | 3695 | CD | GLN | C | 242 | 5.521 | 9.645 | 28.947 | 1.00 | 39.64 | C |
| ATOM | 3696 | OE1 | GLN | C | 242 | 5.234 | 10.007 | 27.806 | 1.00 | 38.87 | O |
| ATOM | 3697 | NE2 | GLN | C | 242 | 6.302 | 10.356 | 29.748 | 1.00 | 37.22 | N |
| ATOM | 3698 | N | LEU | C | 243 | 2.009 | 5.885 | 31.097 | 1.00 | 41.02 | N |
| ATOM | 3699 | CA | LEU | C | 243 | 0.747 | 5.819 | 31.829 | 1.00 | 40.80 | C |
| ATOM | 3700 | C | LEU | C | 243 | 0.183 | 7.216 | 31.908 | 1.00 | 41.12 | C |
| ATOM | 3701 | O | LEU | C | 243 | 0.870 | 8.145 | 32.319 | 1.00 | 41.00 | O |
| ATOM | 3702 | CB | LEU | C | 243 | 0.928 | 5.238 | 33.234 | 1.00 | 40.75 | C |
| ATOM | 3703 | CG | LEU | C | 243 | −0.318 | 5.169 | 34.135 | 1.00 | 39.89 | C |
| ATOM | 3704 | CD1 | LEU | C | 243 | −0.278 | 3.975 | 35.059 | 1.00 | 39.00 | C |
| ATOM | 3705 | CD2 | LEU | C | 243 | −0.497 | 6.444 | 34.942 | 1.00 | 39.71 | C |
| ATOM | 3706 | N | VAL | C | 244 | −1.065 | 7.357 | 31.487 | 1.00 | 41.72 | N |
| ATOM | 3707 | CA | VAL | C | 244 | −1.775 | 8.626 | 31.558 | 1.00 | 42.45 | C |
| ATOM | 3708 | C | VAL | C | 244 | −3.088 | 8.415 | 32.300 | 1.00 | 42.99 | C |
| ATOM | 3709 | O | VAL | C | 244 | −3.850 | 7.506 | 31.987 | 1.00 | 43.26 | O |
| ATOM | 3710 | CB | VAL | C | 244 | −1.988 | 9.251 | 30.160 | 1.00 | 42.49 | C |
| ATOM | 3711 | CG1 | VAL | C | 244 | −0.745 | 10.021 | 29.732 | 1.00 | 43.10 | C |
| ATOM | 3712 | CG2 | VAL | C | 244 | −2.305 | 8.189 | 29.118 | 1.00 | 42.37 | C |
| ATOM | 3713 | N | MET | C | 245 | −3.341 | 9.249 | 33.299 | 1.00 | 43.80 | N |
| ATOM | 3714 | CA | MET | C | 245 | −4.429 | 9.015 | 34.251 | 1.00 | 44.49 | C |
| ATOM | 3715 | C | MET | C | 245 | −5.827 | 9.019 | 33.607 | 1.00 | 44.82 | C |
| ATOM | 3716 | O | MET | C | 245 | −6.798 | 8.561 | 34.209 | 1.00 | 45.00 | O |
| ATOM | 3717 | CB | MET | C | 245 | −4.334 | 10.019 | 35.409 | 1.00 | 44.34 | C |
| ATOM | 3718 | CG | MET | C | 245 | −4.660 | 9.438 | 36.782 | 1.00 | 45.88 | C |
| ATOM | 3719 | SD | MET | C | 245 | −3.658 | 8.010 | 37.287 | 1.00 | 48.81 | S |
| ATOM | 3720 | CE | MET | C | 245 | −2.039 | 8.762 | 37.416 | 1.00 | 48.73 | C |
| ATOM | 3721 | N | GLU | C | 246 | −5.903 | 9.499 | 32.368 | 1.00 | 45.38 | N |
| ATOM | 3722 | CA | GLU | C | 246 | −7.160 | 9.685 | 31.643 | 1.00 | 45.75 | C |
| ATOM | 3723 | C | GLU | C | 246 | −7.332 | 8.661 | 30.535 | 1.00 | 45.55 | C |
| ATOM | 3724 | O | GLU | C | 246 | −8.352 | 8.649 | 29.850 | 1.00 | 45.66 | O |
| ATOM | 3725 | CB | GLU | C | 246 | −7.177 | 11.066 | 30.995 | 1.00 | 46.10 | C |
| ATOM | 3726 | CG | GLU | C | 246 | −6.685 | 12.188 | 31.883 | 1.00 | 48.47 | C |
| ATOM | 3727 | CD | GLU | C | 246 | −7.802 | 12.799 | 32.688 | 1.00 | 51.84 | C |
| ATOM | 3728 | OE1 | GLU | C | 246 | −8.413 | 12.076 | 33.506 | 1.00 | 53.95 | O |
| ATOM | 3729 | OE2 | GLU | C | 246 | −8.071 | 14.009 | 32.503 | 1.00 | 53.36 | O |
| ATOM | 3730 | N | GLY | C | 247 | −6.329 | 7.813 | 30.341 | 1.00 | 45.25 | N |
| ATOM | 3731 | CA | GLY | C | 247 | −6.333 | 6.898 | 29.217 | 1.00 | 44.99 | C |
| ATOM | 3732 | C | GLY | C | 247 | −5.674 | 7.516 | 28.000 | 1.00 | 44.82 | C |
| ATOM | 3733 | O | GLY | C | 247 | −4.956 | 6.839 | 27.260 | 1.00 | 44.83 | O |
| ATOM | 3734 | N | TYR | C | 248 | −5.928 | 8.798 | 27.774 | 1.00 | 44.77 | N |
| ATOM | 3735 | CA | TYR | C | 248 | −5.291 | 9.496 | 26.661 | 1.00 | 44.99 | C |
| ATOM | 3736 | C | TYR | C | 248 | −4.930 | 10.936 | 26.984 | 1.00 | 44.97 | C |
| ATOM | 3737 | O | TYR | C | 248 | −5.490 | 11.566 | 27.893 | 1.00 | 44.98 | O |
| ATOM | 3738 | CB | TYR | C | 248 | −6.128 | 9.420 | 25.372 | 1.00 | 44.78 | C |
| ATOM | 3739 | CG | TYR | C | 248 | −7.265 | 10.410 | 25.307 | 1.00 | 45.12 | C |
| ATOM | 3740 | CD1 | TYR | C | 248 | −8.445 | 10.202 | 26.037 | 1.00 | 45.22 | C |
| ATOM | 3741 | CD2 | TYR | C | 248 | −7.171 | 11.559 | 24.516 | 1.00 | 45.26 | C |
| ATOM | 3742 | CE1 | TYR | C | 248 | −9.503 | 11.119 | 25.978 | 1.00 | 45.30 | C |
| ATOM | 3743 | CE2 | TYR | C | 248 | −8.225 | 12.482 | 24.449 | 1.00 | 45.14 | C |
| ATOM | 3744 | CZ | TYR | C | 248 | −9.385 | 12.251 | 25.180 | 1.00 | 45.21 | C |
| ATOM | 3745 | OH | TYR | C | 248 | −10.423 | 13.151 | 25.117 | 1.00 | 45.09 | O |
| ATOM | 3746 | N | ASN | C | 249 | −3.985 | 11.445 | 26.209 | 1.00 | 45.10 | N |
| ATOM | 3747 | CA | ASN | C | 249 | −3.421 | 12.744 | 26.452 | 1.00 | 45.21 | C |
| ATOM | 3748 | C | ASN | C | 249 | −2.849 | 13.361 | 25.193 | 1.00 | 44.86 | C |
| ATOM | 3749 | O | ASN | C | 249 | −2.134 | 12.712 | 24.434 | 1.00 | 44.67 | O |
| ATOM | 3750 | CB | ASN | C | 249 | −2.328 | 12.642 | 27.508 | 1.00 | 45.52 | C |
| ATOM | 3751 | CG | ASN | C | 249 | −1.798 | 13.981 | 27.909 | 1.00 | 46.16 | C |
| ATOM | 3752 | OD1 | ASN | C | 249 | −2.514 | 14.791 | 28.497 | 1.00 | 48.02 | O |
| ATOM | 3753 | ND2 | ASN | C | 249 | −0.540 | 14.238 | 27.583 | 1.00 | 46.87 | N |
| ATOM | 3754 | N | TRP | C | 250 | −3.184 | 14.626 | 24.985 | 1.00 | 44.68 | N |
| ATOM | 3755 | CA | TRP | C | 250 | −2.579 | 15.419 | 23.936 | 1.00 | 44.28 | C |
| ATOM | 3756 | C | TRP | C | 250 | −1.365 | 16.139 | 24.515 | 1.00 | 44.03 | C |
| ATOM | 3757 | O | TRP | C | 250 | −1.358 | 16.541 | 25.678 | 1.00 | 44.02 | O |
| ATOM | 3758 | CB | TRP | C | 250 | −3.573 | 16.453 | 23.410 | 1.00 | 44.11 | C |
| ATOM | 3759 | CG | TRP | C | 250 | −4.726 | 15.910 | 22.633 | 1.00 | 43.82 | C |
| ATOM | 3760 | CD1 | TRP | C | 250 | −6.012 | 15.787 | 23.062 | 1.00 | 43.64 | C |
| ATOM | 3761 | CD2 | TRP | C | 250 | −4.708 | 15.447 | 21.277 | 1.00 | 44.53 | C |
| ATOM | 3762 | NE1 | TRP | C | 250 | −6.800 | 15.268 | 22.063 | 1.00 | 43.57 | N |
| ATOM | 3763 | CE2 | TRP | C | 250 | −6.026 | 15.051 | 20.954 | 1.00 | 44.14 | C |
| ATOM | 3764 | CE3 | TRP | C | 250 | −3.707 | 15.330 | 20.297 | 1.00 | 44.70 | C |
| ATOM | 3765 | CZ2 | TRP | C | 250 | −6.371 | 14.546 | 19.692 | 1.00 | 43.99 | C |
| ATOM | 3766 | CZ3 | TRP | C | 250 | −4.055 | 14.828 | 19.039 | 1.00 | 43.83 | C |
| ATOM | 3767 | CH2 | TRP | C | 250 | −5.375 | 14.443 | 18.753 | 1.00 | 43.69 | C |
| ATOM | 3768 | N | CYS | C | 251 | −0.332 | 16.282 | 23.703 | 1.00 | 43.74 | N |
| ATOM | 3769 | CA | CYS | C | 251 | 0.797 | 17.113 | 24.070 | 1.00 | 43.95 | C |
| ATOM | 3770 | C | CYS | C | 251 | 1.426 | 17.668 | 22.794 | 1.00 | 43.70 | C |
| ATOM | 3771 | O | CYS | C | 251 | 0.979 | 17.343 | 21.687 | 1.00 | 43.61 | O |
| ATOM | 3772 | CB | CYS | C | 251 | 1.809 | 16.326 | 24.914 | 1.00 | 43.85 | C |
| ATOM | 3773 | SG | CYS | C | 251 | 2.758 | 15.112 | 23.985 | 1.00 | 44.69 | S |
| ATOM | 3774 | N | HIS | C | 252 | 2.438 | 18.517 | 22.950 | 1.00 | 43.50 | N |
| ATOM | 3775 | CA | HIS | C | 252 | 3.157 | 19.073 | 21.804 | 1.00 | 43.77 | C |
| ATOM | 3776 | C | HIS | C | 252 | 2.214 | 19.721 | 20.796 | 1.00 | 44.09 | C |
| ATOM | 3777 | O | HIS | C | 252 | 2.424 | 19.623 | 19.575 | 1.00 | 43.93 | O |
| ATOM | 3778 | CB | HIS | C | 252 | 3.997 | 17.989 | 21.134 | 1.00 | 43.58 | C |
| ATOM | 3779 | CG | HIS | C | 252 | 5.169 | 17.554 | 21.950 | 1.00 | 43.35 | C |
| ATOM | 3780 | ND1 | HIS | C | 252 | 6.176 | 16.767 | 21.438 | 1.00 | 42.53 | N |
| ATOM | 3781 | CD2 | HIS | C | 252 | 5.508 | 17.818 | 23.235 | 1.00 | 42.69 | C |
| ATOM | 3782 | CE1 | HIS | C | 252 | 7.077 | 16.549 | 22.379 | 1.00 | 43.59 | C |
| ATOM | 3783 | NE2 | HIS | C | 252 | 6.698 | 17.181 | 23.477 | 1.00 | 42.91 | N |
| ATOM | 3784 | N | ASP | C | 253 | 1.172 | 20.369 | 21.330 | 1.00 | 44.33 | N |
| ATOM | 3785 | CA | ASP | C | 253 | 0.178 | 21.094 | 20.540 | 1.00 | 44.46 | C |
| ATOM | 3786 | C | ASP | C | 253 | −0.511 | 20.174 | 19.533 | 1.00 | 44.51 | C |
| ATOM | 3787 | O | ASP | C | 253 | −0.660 | 20.510 | 18.361 | 1.00 | 44.55 | O |
| ATOM | 3788 | CB | ASP | C | 253 | 0.828 | 22.300 | 19.847 | 1.00 | 44.58 | C |
| ATOM | 3789 | CG | ASP | C | 253 | −0.184 | 23.265 | 19.273 | 1.00 | 44.97 | C |
| ATOM | 3790 | OD1 | ASP | C | 253 | −1.303 | 23.359 | 19.831 | 1.00 | 44.76 | O |
| ATOM | 3791 | OD2 | ASP | C | 253 | 0.153 | 23.931 | 18.265 | 1.00 | 45.19 | O |
| ATOM | 3792 | N | ARG | C | 254 | −0.912 | 18.998 | 20.006 | 1.00 | 44.57 | N |
| ATOM | 3793 | CA | ARG | C | 254 | −1.627 | 18.015 | 19.187 | 1.00 | 44.53 | C |
| ATOM | 3794 | C | ARG | C | 254 | −0.807 | 17.395 | 18.029 | 1.00 | 44.30 | C |
| ATOM | 3795 | O | ARG | C | 254 | −1.367 | 16.865 | 17.061 | 1.00 | 44.14 | O |
| ATOM | 3796 | CB | ARG | C | 254 | −2.983 | 18.574 | 18.709 | 1.00 | 44.58 | C |
| ATOM | 3797 | CG | ARG | C | 254 | −3.862 | 19.053 | 19.852 | 1.00 | 44.86 | C |
| ATOM | 3798 | CD | ARG | C | 254 | −5.331 | 18.762 | 19.631 | 1.00 | 45.38 | C |
| ATOM | 3799 | NE | ARG | C | 254 | −6.094 | 19.125 | 20.824 | 1.00 | 47.30 | N |
| ATOM | 3800 | CZ | ARG | C | 254 | −7.361 | 18.784 | 21.058 | 1.00 | 48.16 | C |
| ATOM | 3801 | NH1 | ARG | C | 254 | −8.039 | 18.059 | 20.174 | 1.00 | 48.34 | N |
| ATOM | 3802 | NH2 | ARG | C | 254 | −7.955 | 19.172 | 22.183 | 1.00 | 47.86 | N |
| ATOM | 3803 | N | ASN | C | 255 | 0.514 | 17.453 | 18.137 | 1.00 | 44.11 | N |
| ATOM | 3804 | CA | ASN | C | 255 | 1.368 | 16.738 | 17.195 | 1.00 | 44.29 | C |
| ATOM | 3805 | C | ASN | C | 255 | 1.542 | 15.301 | 17.668 | 1.00 | 44.33 | C |
| ATOM | 3806 | O | ASN | C | 255 | 1.803 | 14.393 | 16.871 | 1.00 | 44.49 | O |
| ATOM | 3807 | CB | ASN | C | 255 | 2.726 | 17.431 | 17.037 | 1.00 | 44.30 | C |
| ATOM | 3808 | CG | ASN | C | 255 | 2.666 | 18.638 | 16.103 | 1.00 | 44.95 | C |
| ATOM | 3809 | OD1 | ASN | C | 255 | 2.707 | 18.493 | 14.877 | 1.00 | 44.75 | O |
| ATOM | 3810 | ND2 | ASN | C | 255 | 2.586 | 19.840 | 16.684 | 1.00 | 45.08 | N |
| ATOM | 3811 | N | VAL | C | 256 | 1.382 | 15.117 | 18.980 | 1.00 | 44.20 | N |
| ATOM | 3812 | CA | VAL | C | 256 | 1.505 | 13.823 | 19.640 | 1.00 | 43.79 | C |
| ATOM | 3813 | C | VAL | C | 256 | 0.254 | 13.556 | 20.482 | 1.00 | 43.86 | C |
| ATOM | 3814 | O | VAL | C | 256 | −0.284 | 14.465 | 21.122 | 1.00 | 43.83 | O |
| ATOM | 3815 | CB | VAL | C | 256 | 2.752 | 13.784 | 20.569 | 1.00 | 43.67 | C |
| ATOM | 3816 | CG1 | VAL | C | 256 | 2.857 | 12.453 | 21.311 | 1.00 | 43.06 | C |
| ATOM | 3817 | CG2 | VAL | C | 256 | 4.023 | 14.055 | 19.786 | 1.00 | 43.60 | C |
| ATOM | 3818 | N | VAL | C | 257 | −0.210 | 12.310 | 20.466 | 1.00 | 43.66 | N |
| ATOM | 3819 | CA | VAL | C | 257 | −1.172 | 11.846 | 21.451 | 1.00 | 43.65 | C |
| ATOM | 3820 | C | VAL | C | 257 | −0.619 | 10.577 | 22.104 | 1.00 | 43.99 | C |
| ATOM | 3821 | O | VAL | C | 257 | 0.034 | 9.763 | 21.439 | 1.00 | 44.01 | O |
| ATOM | 3822 | CB | VAL | C | 257 | −2.616 | 11.645 | 20.859 | 1.00 | 43.65 | C |
| ATOM | 3823 | CG1 | VAL | C | 257 | −2.657 | 10.539 | 19.796 | 1.00 | 43.11 | C |
| ATOM | 3824 | CG2 | VAL | C | 257 | −3.626 | 11.362 | 21.967 | 1.00 | 42.84 | C |
| ATOM | 3825 | N | THR | C | 258 | −0.850 | 10.433 | 23.409 | 1.00 | 44.18 | N |
| ATOM | 3826 | CA | THR | C | 258 | −0.455 | 9.225 | 24.129 | 1.00 | 44.31 | C |
| ATOM | 3827 | C | THR | C | 258 | −1.715 | 8.452 | 24.477 | 1.00 | 44.59 | C |
| ATOM | 3828 | O | THR | C | 258 | −2.667 | 9.016 | 25.008 | 1.00 | 44.75 | O |
| ATOM | 3829 | CB | THR | C | 258 | 0.400 | 9.524 | 25.402 | 1.00 | 44.05 | C |
| ATOM | 3830 | OG1 | THR | C | 258 | 1.592 | 10.210 | 25.027 | 1.00 | 43.59 | O |
| ATOM | 3831 | CG2 | THR | C | 258 | 0.816 | 8.245 | 26.109 | 1.00 | 43.91 | C |
| ATOM | 3832 | N | ILE | C | 259 | −1.710 | 7.165 | 24.150 | 1.00 | 44.84 | N |
| ATOM | 3833 | CA | ILE | C | 259 | −2.851 | 6.294 | 24.390 | 1.00 | 45.17 | C |
| ATOM | 3834 | C | ILE | C | 259 | −2.402 | 5.131 | 25.266 | 1.00 | 45.41 | C |
| ATOM | 3835 | O | ILE | C | 259 | −1.561 | 4.332 | 24.859 | 1.00 | 45.81 | O |
| ATOM | 3836 | CB | ILE | C | 259 | −3.442 | 5.753 | 23.068 | 1.00 | 45.05 | C |
| ATOM | 3837 | CG1 | ILE | C | 259 | −3.901 | 6.905 | 22.166 | 1.00 | 44.63 | C |
| ATOM | 3838 | CG2 | ILE | C | 259 | −4.597 | 4.813 | 23.356 | 1.00 | 45.67 | C |
| ATOM | 3839 | CD1 | ILE | C | 259 | −3.988 | 6.558 | 20.697 | 1.00 | 43.05 | C |
| ATOM | 3840 | N | PHE | C | 260 | −2.947 | 5.062 | 26.475 | 1.00 | 45.36 | N |
| ATOM | 3841 | CA | PHE | C | 260 | −2.642 | 3.989 | 27.405 | 1.00 | 45.32 | C |
| ATOM | 3842 | C | PHE | C | 260 | −3.915 | 3.165 | 27.581 | 1.00 | 45.47 | C |
| ATOM | 3843 | O | PHE | C | 260 | −4.969 | 3.708 | 27.957 | 1.00 | 45.40 | O |
| ATOM | 3844 | CB | PHE | C | 260 | −2.179 | 4.573 | 28.738 | 1.00 | 45.18 | C |
| ATOM | 3845 | CG | PHE | C | 260 | −1.474 | 3.588 | 29.643 | 1.00 | 44.97 | C |
| ATOM | 3846 | CD1 | PHE | C | 260 | −0.187 | 3.144 | 29.350 | 1.00 | 44.35 | C |
| ATOM | 3847 | CD2 | PHE | C | 260 | −2.083 | 3.147 | 30.814 | 1.00 | 43.92 | C |
| ATOM | 3848 | CE1 | PHE | C | 260 | 0.465 | 2.264 | 30.200 | 1.00 | 44.72 | C |
| ATOM | 3849 | CE2 | PHE | C | 260 | −1.435 | 2.267 | 31.667 | 1.00 | 43.79 | C |
| ATOM | 3850 | CZ | PHE | C | 260 | −0.162 | 1.825 | 31.363 | 1.00 | 44.17 | C |
| ATOM | 3851 | N | SER | C | 261 | −3.812 | 1.864 | 27.301 | 1.00 | 45.13 | N |
| ATOM | 3852 | CA | SER | C | 261 | −4.986 | 1.013 | 27.208 | 1.00 | 45.04 | C |
| ATOM | 3853 | C | SER | C | 261 | −5.101 | −0.017 | 28.331 | 1.00 | 45.20 | C |
| ATOM | 3854 | O | SER | C | 261 | −5.868 | −0.967 | 28.222 | 1.00 | 45.45 | O |
| ATOM | 3855 | CB | SER | C | 261 | −5.022 | 0.328 | 25.842 | 1.00 | 45.02 | C |
| ATOM | 3856 | OG | SER | C | 261 | −5.094 | 1.285 | 24.802 | 1.00 | 44.65 | O |
| ATOM | 3857 | N | ALA | C | 262 | −4.356 | 0.176 | 29.414 | 1.00 | 45.29 | N |
| ATOM | 3858 | CA | ALA | C | 262 | −4.444 | −0.724 | 30.564 | 1.00 | 45.10 | C |
| ATOM | 3859 | C | ALA | C | 262 | −5.064 | 0.001 | 31.760 | 1.00 | 45.04 | C |
| ATOM | 3860 | O | ALA | C | 262 | −4.380 | 0.768 | 32.445 | 1.00 | 45.49 | O |
| ATOM | 3861 | CB | ALA | C | 262 | −3.072 | −1.274 | 30.912 | 1.00 | 45.05 | C |
| ATOM | 3862 | N | PRO | C | 263 | −6.367 | −0.222 | 32.010 | 1.00 | 44.73 | N |
| ATOM | 3863 | CA | PRO | C | 263 | −7.052 | 0.493 | 33.080 | 1.00 | 44.81 | C |
| ATOM | 3864 | C | PRO | C | 263 | −6.586 | 0.023 | 34.435 | 1.00 | 44.93 | C |
| ATOM | 3865 | O | PRO | C | 263 | −6.241 | −1.142 | 34.588 | 1.00 | 45.31 | O |
| ATOM | 3866 | CB | PRO | C | 263 | −8.514 | 0.107 | 32.880 | 1.00 | 44.84 | C |
| ATOM | 3867 | CG | PRO | C | 263 | −8.482 | −1.167 | 32.183 | 1.00 | 44.47 | C |
| ATOM | 3868 | CD | PRO | C | 263 | −7.267 | −1.150 | 31.314 | 1.00 | 44.68 | C |
| ATOM | 3869 | N | ASN | C | 264 | −6.583 | 0.924 | 35.408 | 1.00 | 45.03 | N |
| ATOM | 3870 | CA | ASN | C | 264 | −6.097 | 0.611 | 36.748 | 1.00 | 45.40 | C |
| ATOM | 3871 | C | ASN | C | 264 | −4.823 | −0.233 | 36.697 | 1.00 | 45.92 | C |
| ATOM | 3872 | O | ASN | C | 264 | −4.755 | −1.310 | 37.299 | 1.00 | 46.03 | O |
| ATOM | 3873 | CB | ASN | C | 264 | −7.188 | −0.081 | 37.579 | 1.00 | 45.14 | C |
| ATOM | 3874 | CG | ASN | C | 264 | −6.861 | −0.115 | 39.059 | 1.00 | 43.96 | C |
| ATOM | 3875 | OD1 | ASN | C | 264 | −6.335 | 0.842 | 39.616 | 1.00 | 42.89 | O |
| ATOM | 3876 | ND2 | ASN | C | 264 | −7.169 | −1.223 | 39.697 | 1.00 | 42.41 | N |
| ATOM | 3877 | N | TYR | C | 265 | −3.824 | 0.272 | 35.968 | 1.00 | 46.55 | N |
| ATOM | 3878 | CA | TYR | C | 265 | −2.595 | −0.466 | 35.690 | 1.00 | 47.17 | C |
| ATOM | 3879 | C | TYR | C | 265 | −2.009 | −1.149 | 36.926 | 1.00 | 47.92 | C |
| ATOM | 3880 | O | TYR | C | 265 | −1.896 | −0.530 | 38.002 | 1.00 | 47.80 | O |
| ATOM | 3881 | CB | TYR | C | 265 | −1.530 | 0.410 | 35.012 | 1.00 | 47.02 | C |
| ATOM | 3882 | CG | TYR | C | 265 | −0.222 | −0.330 | 34.863 | 1.00 | 46.94 | C |
| ATOM | 3883 | CD1 | TYR | C | 265 | 0.874 | −0.008 | 35.660 | 1.00 | 46.89 | C |
| ATOM | 3884 | CD2 | TYR | C | 265 | −0.097 | −1.394 | 33.964 | 1.00 | 46.28 | C |
| ATOM | 3885 | CE1 | TYR | C | 265 | 2.072 | −0.707 | 35.557 | 1.00 | 46.55 | C |
| ATOM | 3886 | CE2 | TYR | C | 265 | 1.094 | −2.096 | 33.853 | 1.00 | 46.96 | C |
| ATOM | 3887 | CZ | TYR | C | 265 | 2.175 | −1.746 | 34.659 | 1.00 | 46.93 | C |
| ATOM | 3888 | OH | TYR | C | 265 | 3.360 | −2.435 | 34.572 | 1.00 | 47.16 | O |
| ATOM | 3889 | N | CYS | C | 266 | −1.657 | −2.430 | 36.750 | 1.00 | 48.40 | N |
| ATOM | 3890 | CA | CYS | C | 266 | −1.097 | −3.258 | 37.812 | 1.00 | 49.08 | C |
| ATOM | 3891 | C | CYS | C | 266 | −2.030 | −3.367 | 39.016 | 1.00 | 49.23 | C |
| ATOM | 3892 | O | CYS | C | 266 | −1.577 | −3.529 | 40.150 | 1.00 | 49.34 | O |
| ATOM | 3893 | CB | CYS | C | 266 | 0.269 | −2.721 | 38.245 | 1.00 | 49.19 | C |
| ATOM | 3894 | SG | CYS | C | 266 | 1.634 | −3.840 | 37.935 | 1.00 | 51.10 | S |
| ATOM | 3895 | N | TYR | C | 267 | −3.331 | −3.249 | 38.754 | 1.00 | 49.51 | N |
| ATOM | 3896 | CA | TYR | C | 267 | −4.385 | −3.391 | 39.766 | 1.00 | 49.85 | C |
| ATOM | 3897 | C | TYR | C | 267 | −4.284 | −2.392 | 40.930 | 1.00 | 49.56 | C |
| ATOM | 3898 | O | TYR | C | 267 | −4.880 | −2.614 | 41.988 | 1.00 | 49.68 | O |
| ATOM | 3899 | CB | TYR | C | 267 | −4.469 | −4.841 | 40.307 | 1.00 | 50.13 | C |
| ATOM | 3900 | CG | TYR | C | 267 | −4.548 | −5.966 | 39.271 | 1.00 | 50.85 | C |
| ATOM | 3901 | CD1 | TYR | C | 267 | −4.503 | −7.306 | 39.670 | 1.00 | 50.83 | C |
| ATOM | 3902 | CD2 | TYR | C | 267 | −4.660 | −5.697 | 37.902 | 1.00 | 51.57 | C |
| ATOM | 3903 | CE1 | TYR | C | 267 | −4.580 | −8.348 | 38.731 | 1.00 | 51.93 | C |
| ATOM | 3904 | CE2 | TYR | C | 267 | −4.723 | −6.721 | 36.957 | 1.00 | 51.40 | C |
| ATOM | 3905 | CZ | TYR | C | 267 | −4.687 | −8.042 | 37.373 | 1.00 | 51.95 | C |
| ATOM | 3906 | OH | TYR | C | 267 | −4.749 | −9.050 | 36.425 | 1.00 | 52.13 | O |
| ATOM | 3907 | N | ARG | C | 268 | −3.547 | −1.295 | 40.748 | 1.00 | 49.10 | N |
| ATOM | 3908 | CA | ARG | C | 268 | −3.422 | −0.303 | 41.830 | 1.00 | 48.85 | C |
| ATOM | 3909 | C | ARG | C | 268 | −3.386 | 1.171 | 41.425 | 1.00 | 47.68 | C |
| ATOM | 3910 | O | ARG | C | 268 | −3.619 | 2.042 | 42.257 | 1.00 | 47.64 | O |
| ATOM | 3911 | CB | ARG | C | 268 | −2.252 | −0.636 | 42.778 | 1.00 | 48.77 | C |
| ATOM | 3912 | CG | ARG | C | 268 | −0.898 | −0.884 | 42.114 | 1.00 | 49.84 | C |
| ATOM | 3913 | CD | ARG | C | 268 | 0.108 | −1.403 | 43.146 | 1.00 | 50.68 | C |
| ATOM | 3914 | NE | ARG | C | 268 | 0.859 | −2.572 | 42.673 | 1.00 | 54.00 | N |
| ATOM | 3915 | CZ | ARG | C | 268 | 2.157 | −2.576 | 42.377 | 1.00 | 55.09 | C |
| ATOM | 3916 | NH1 | ARG | C | 268 | 2.879 | −1.469 | 42.507 | 1.00 | 55.44 | N |
| ATOM | 3917 | NH2 | ARG | C | 268 | 2.738 | −3.695 | 41.952 | 1.00 | 55.38 | N |
| ATOM | 3918 | N | CYS | C | 269 | −3.131 | 1.461 | 40.157 | 1.00 | 46.78 | N |
| ATOM | 3919 | CA | CYS | C | 269 | −2.922 | 2.855 | 39.738 | 1.00 | 45.80 | C |
| ATOM | 3920 | C | CYS | C | 269 | −4.176 | 3.765 | 39.700 | 1.00 | 44.95 | C |
| ATOM | 3921 | O | CYS | C | 269 | −4.085 | 4.951 | 40.005 | 1.00 | 44.57 | O |
| ATOM | 3922 | CB | CYS | C | 269 | −2.125 | 2.909 | 38.425 | 1.00 | 45.98 | C |
| ATOM | 3923 | SG | CYS | C | 269 | −0.432 | 2.211 | 38.565 | 1.00 | 46.12 | S |
| ATOM | 3924 | N | GLY | C | 270 | −5.336 | 3.218 | 39.341 | 1.00 | 44.09 | N |
| ATOM | 3925 | CA | GLY | C | 270 | −6.569 | 4.007 | 39.305 | 1.00 | 43.01 | C |
| ATOM | 3926 | C | GLY | C | 270 | −6.761 | 4.837 | 38.045 | 1.00 | 42.59 | C |
| ATOM | 3927 | O | GLY | C | 270 | −7.699 | 5.628 | 37.956 | 1.00 | 42.51 | O |
| ATOM | 3928 | N | ASN | C | 271 | −5.876 | 4.661 | 37.067 | 1.00 | 42.18 | N |
| ATOM | 3929 | CA | ASN | C | 271 | −5.964 | 5.362 | 35.794 | 1.00 | 41.61 | C |
| ATOM | 3930 | C | ASN | C | 271 | −7.080 | 4.794 | 34.943 | 1.00 | 41.80 | C |
| ATOM | 3931 | O | ASN | C | 271 | −7.462 | 3.629 | 35.088 | 1.00 | 41.88 | O |
| ATOM | 3932 | CB | ASN | C | 271 | −4.666 | 5.202 | 35.017 | 1.00 | 41.26 | C |
| ATOM | 3933 | CG | ASN | C | 271 | −4.297 | 3.753 | 34.825 | 1.00 | 40.36 | C |
| ATOM | 3934 | OD1 | ASN | C | 271 | −4.017 | 3.063 | 35.794 | 1.00 | 39.76 | O |
| ATOM | 3935 | ND2 | ASN | C | 271 | −4.315 | 3.276 | 33.581 | 1.00 | 38.16 | N |
| ATOM | 3936 | N | GLN | C | 272 | −7.589 | 5.626 | 34.045 | 1.00 | 41.74 | N |
| ATOM | 3937 | CA | GLN | C | 272 | −8.520 | 5.184 | 33.036 | 1.00 | 41.81 | C |
| ATOM | 3938 | C | GLN | C | 272 | −7.731 | 4.658 | 31.848 | 1.00 | 42.16 | C |
| ATOM | 3939 | O | GLN | C | 272 | −6.553 | 4.994 | 31.673 | 1.00 | 42.26 | O |
| ATOM | 3940 | CB | GLN | C | 272 | −9.386 | 6.351 | 32.592 | 1.00 | 41.64 | C |
| ATOM | 3941 | CG | GLN | C | 272 | −10.416 | 6.777 | 33.592 | 1.00 | 41.99 | C |
| ATOM | 3942 | CD | GLN | C | 272 | −11.458 | 7.669 | 32.967 | 1.00 | 43.44 | C |
| ATOM | 3943 | OE1 | GLN | C | 272 | −11.251 | 8.874 | 32.811 | 1.00 | 44.15 | O |
| ATOM | 3944 | NE2 | GLN | C | 272 | −12.595 | 7.082 | 32.601 | 1.00 | 44.01 | N |
| ATOM | 3945 | N | ALA | C | 273 | −8.374 | 3.821 | 31.038 | 1.00 | 42.36 | N |
| ATOM | 3946 | CA | ALA | C | 273 | −7.790 | 3.397 | 29.767 | 1.00 | 42.40 | C |
| ATOM | 3947 | C | ALA | C | 273 | −8.503 | 4.098 | 28.618 | 1.00 | 42.36 | C |
| ATOM | 3948 | O | ALA | C | 273 | −9.569 | 4.683 | 28.814 | 1.00 | 42.17 | O |
| ATOM | 3949 | CB | ALA | C | 273 | −7.879 | 1.900 | 29.616 | 1.00 | 42.36 | C |
| ATOM | 3950 | N | ALA | C | 274 | −7.904 | 4.055 | 27.430 | 1.00 | 42.51 | N |
| ATOM | 3951 | CA | ALA | C | 274 | −8.504 | 4.664 | 26.239 | 1.00 | 42.78 | C |
| ATOM | 3952 | C | ALA | C | 274 | −8.053 | 3.997 | 24.958 | 1.00 | 42.98 | C |
| ATOM | 3953 | O | ALA | C | 274 | −6.948 | 3.467 | 24.873 | 1.00 | 43.13 | O |
| ATOM | 3954 | CB | ALA | C | 274 | −8.221 | 6.174 | 26.170 | 1.00 | 42.59 | C |
| ATOM | 3955 | N | ILE | C | 275 | −8.940 | 4.032 | 23.971 | 1.00 | 43.21 | N |
| ATOM | 3956 | CA | ILE | C | 275 | −8.636 | 3.640 | 22.604 | 1.00 | 43.20 | C |
| ATOM | 3957 | C | ILE | C | 275 | −9.091 | 4.764 | 21.668 | 1.00 | 43.75 | C |
| ATOM | 3958 | O | ILE | C | 275 | −10.028 | 5.508 | 21.984 | 1.00 | 43.67 | O |
| ATOM | 3959 | CB | ILE | C | 275 | −9.333 | 2.317 | 22.237 | 1.00 | 43.13 | C |
| ATOM | 3960 | CG1 | ILE | C | 275 | −10.823 | 2.368 | 22.631 | 1.00 | 42.55 | C |
| ATOM | 3961 | CG2 | ILE | C | 275 | −8.606 | 1.131 | 22.897 | 1.00 | 42.46 | C |
| ATOM | 3962 | CD1 | ILE | C | 275 | −11.695 | 1.316 | 21.982 | 1.00 | 40.46 | C |
| ATOM | 3963 | N | MET | C | 276 | −8.425 | 4.897 | 20.526 | 1.00 | 44.25 | N |
| ATOM | 3964 | CA | MET | C | 276 | −8.768 | 5.945 | 19.570 | 1.00 | 44.73 | C |
| ATOM | 3965 | C | MET | C | 276 | −9.356 | 5.368 | 18.291 | 1.00 | 45.02 | C |
| ATOM | 3966 | O | MET | C | 276 | −8.657 | 4.685 | 17.534 | 1.00 | 45.27 | O |
| ATOM | 3967 | CB | MET | C | 276 | −7.539 | 6.787 | 19.244 | 1.00 | 44.64 | C |
| ATOM | 3968 | CG | MET | C | 276 | −7.798 | 7.916 | 18.267 | 1.00 | 44.56 | C |
| ATOM | 3969 | SD | MET | C | 276 | −6.320 | 8.911 | 18.002 | 1.00 | 45.17 | S |
| ATOM | 3970 | CE | MET | C | 276 | −5.332 | 7.857 | 16.929 | 1.00 | 43.41 | C |
| ATOM | 3971 | N | GLU | C | 277 | −10.634 | 5.656 | 18.052 | 1.00 | 45.10 | N |
| ATOM | 3972 | CA | GLU | C | 277 | −11.307 | 5.205 | 16.839 | 1.00 | 45.16 | C |
| ATOM | 3973 | C | GLU | C | 277 | −11.061 | 6.184 | 15.717 | 1.00 | 44.97 | C |
| ATOM | 3974 | O | GLU | C | 277 | −11.310 | 7.383 | 15.861 | 1.00 | 45.05 | O |
| ATOM | 3975 | CB | GLU | C | 277 | −12.817 | 5.072 | 17.052 | 1.00 | 45.36 | C |
| ATOM | 3976 | CG | GLU | C | 277 | −13.217 | 4.361 | 18.338 | 1.00 | 46.09 | C |
| ATOM | 3977 | CD | GLU | C | 277 | −14.629 | 3.810 | 18.300 | 1.00 | 46.46 | C |
| ATOM | 3978 | OE1 | GLU | C | 277 | −15.591 | 4.582 | 18.087 | 1.00 | 47.01 | O |
| ATOM | 3979 | OE2 | GLU | C | 277 | −14.773 | 2.592 | 18.502 | 1.00 | 46.87 | O |
| ATOM | 3980 | N | LEU | C | 278 | −10.570 | 5.659 | 14.602 | 1.00 | 44.91 | N |
| ATOM | 3981 | CA | LEU | C | 278 | −10.425 | 6.430 | 13.371 | 1.00 | 44.93 | C |
| ATOM | 3982 | C | LEU | C | 278 | −11.449 | 5.923 | 12.357 | 1.00 | 44.92 | C |
| ATOM | 3983 | O | LEU | C | 278 | −11.412 | 4.762 | 11.957 | 1.00 | 44.69 | O |
| ATOM | 3984 | CB | LEU | C | 278 | −9.002 | 6.296 | 12.814 | 1.00 | 44.80 | C |
| ATOM | 3985 | CG | LEU | C | 278 | −7.833 | 6.674 | 13.725 | 1.00 | 44.80 | C |
| ATOM | 3986 | CD1 | LEU | C | 278 | −6.505 | 6.262 | 13.114 | 1.00 | 44.72 | C |
| ATOM | 3987 | CD2 | LEU | C | 278 | −7.842 | 8.159 | 14.035 | 1.00 | 45.86 | C |
| ATOM | 3988 | N | ASP | C | 279 | −12.368 | 6.788 | 11.944 | 1.00 | 45.08 | N |
| ATOM | 3989 | CA | ASP | C | 279 | −13.431 | 6.338 | 11.065 | 1.00 | 45.55 | C |
| ATOM | 3990 | C | ASP | C | 279 | −12.987 | 6.198 | 9.604 | 1.00 | 45.76 | C |
| ATOM | 3991 | O | ASP | C | 279 | −11.792 | 6.221 | 9.286 | 1.00 | 45.61 | O |
| ATOM | 3992 | CB | ASP | C | 279 | −14.696 | 7.199 | 11.223 | 1.00 | 45.69 | C |
| ATOM | 3993 | CG | ASP | C | 279 | −14.570 | 8.584 | 10.593 | 1.00 | 46.69 | C |
| ATOM | 3994 | OD1 | ASP | C | 279 | −13.504 | 8.924 | 10.035 | 1.00 | 48.29 | O |
| ATOM | 3995 | OD2 | ASP | C | 279 | −15.563 | 9.339 | 10.652 | 1.00 | 47.11 | O |
| ATOM | 3996 | N | ASP | C | 280 | −13.974 | 6.061 | 8.728 | 1.00 | 46.12 | N |
| ATOM | 3997 | CA | ASP | C | 280 | −13.764 | 5.871 | 7.299 | 1.00 | 46.33 | C |
| ATOM | 3998 | C | ASP | C | 280 | −13.079 | 7.063 | 6.630 | 1.00 | 46.59 | C |
| ATOM | 3999 | O | ASP | C | 280 | −12.548 | 6.929 | 5.527 | 1.00 | 46.67 | O |
| ATOM | 4000 | CB | ASP | C | 280 | −15.106 | 5.579 | 6.608 | 1.00 | 46.20 | C |
| ATOM | 4001 | CG | ASP | C | 280 | −15.872 | 4.425 | 7.255 | 1.00 | 45.72 | C |
| ATOM | 4002 | OD1 | ASP | C | 280 | −15.820 | 4.266 | 8.498 | 1.00 | 45.09 | O |
| ATOM | 4003 | OD2 | ASP | C | 280 | −16.546 | 3.682 | 6.513 | 1.00 | 45.30 | O |
| ATOM | 4004 | N | THR | C | 281 | −13.099 | 8.222 | 7.289 | 1.00 | 46.82 | N |
| ATOM | 4005 | CA | THR | C | 281 | −12.457 | 9.425 | 6.751 | 1.00 | 47.09 | C |
| ATOM | 4006 | C | THR | C | 281 | −11.372 | 9.965 | 7.677 | 1.00 | 47.32 | C |
| ATOM | 4007 | O | THR | C | 281 | −10.972 | 11.121 | 7.543 | 1.00 | 47.30 | O |
| ATOM | 4008 | CB | THR | C | 281 | −13.470 | 10.556 | 6.472 | 1.00 | 47.01 | C |
| ATOM | 4009 | OG1 | THR | C | 281 | −14.159 | 10.886 | 7.682 | 1.00 | 47.46 | O |
| ATOM | 4010 | CG2 | THR | C | 281 | −14.474 | 10.145 | 5.409 | 1.00 | 46.85 | C |
| ATOM | 4011 | N | LEU | C | 282 | −10.902 | 9.126 | 8.605 | 1.00 | 47.62 | N |
| ATOM | 4012 | CA | LEU | C | 282 | −9.861 | 9.495 | 9.580 | 1.00 | 47.80 | C |
| ATOM | 4013 | C | LEU | C | 282 | −10.284 | 10.545 | 10.621 | 1.00 | 48.00 | C |
| ATOM | 4014 | O | LEU | C | 282 | −9.452 | 11.324 | 11.089 | 1.00 | 48.17 | O |
| ATOM | 4015 | CB | LEU | C | 282 | −8.562 | 9.949 | 8.885 | 1.00 | 47.78 | C |
| ATOM | 4016 | CG | LEU | C | 282 | −7.413 | 8.999 | 8.541 | 1.00 | 47.77 | C |
| ATOM | 4017 | CD1 | LEU | C | 282 | −6.198 | 9.828 | 8.115 | 1.00 | 48.04 | C |
| ATOM | 4018 | CD2 | LEU | C | 282 | −7.038 | 8.097 | 9.701 | 1.00 | 47.74 | C |
| ATOM | 4019 | N | LYS | C | 283 | −11.565 | 10.568 | 10.983 | 1.00 | 48.26 | N |
| ATOM | 4020 | CA | LYS | C | 283 | −12.027 | 11.389 | 12.103 | 1.00 | 48.50 | C |
| ATOM | 4021 | C | LYS | C | 283 | −11.722 | 10.637 | 13.403 | 1.00 | 48.72 | C |
| ATOM | 4022 | O | LYS | C | 283 | −12.093 | 9.467 | 13.555 | 1.00 | 48.62 | O |
| ATOM | 4023 | CB | LYS | C | 283 | −13.533 | 11.685 | 12.004 | 1.00 | 48.59 | C |
| ATOM | 4024 | CG | LYS | C | 283 | −14.003 | 12.424 | 10.737 | 1.00 | 48.48 | C |
| ATOM | 4025 | CD | LYS | C | 283 | −15.517 | 12.619 | 10.778 | 1.00 | 48.60 | C |
| ATOM | 4026 | CE | LYS | C | 283 | −16.122 | 12.894 | 9.404 | 1.00 | 48.91 | C |
| ATOM | 4027 | NZ | LYS | C | 283 | −16.137 | 14.343 | 9.042 | 1.00 | 48.77 | N |
| ATOM | 4028 | N | TYR | C | 284 | −11.029 | 11.302 | 14.326 | 1.00 | 49.01 | N |
| ATOM | 4029 | CA | TYR | C | 284 | −10.687 | 10.689 | 15.614 | 1.00 | 49.23 | C |
| ATOM | 4030 | C | TYR | C | 284 | −11.812 | 10.835 | 16.625 | 1.00 | 48.85 | C |
| ATOM | 4031 | O | TYR | C | 284 | −12.489 | 11.861 | 16.671 | 1.00 | 48.79 | O |
| ATOM | 4032 | CB | TYR | C | 284 | −9.374 | 11.257 | 16.194 | 1.00 | 49.83 | C |
| ATOM | 4033 | CG | TYR | C | 284 | −9.350 | 12.766 | 16.427 | 1.00 | 50.51 | C |
| ATOM | 4034 | CD1 | TYR | C | 284 | −10.024 | 13.343 | 17.507 | 1.00 | 51.06 | C |
| ATOM | 4035 | CD2 | TYR | C | 284 | −8.631 | 13.606 | 15.583 | 1.00 | 50.74 | C |
| ATOM | 4036 | CE1 | TYR | C | 284 | −10.005 | 14.712 | 17.724 | 1.00 | 50.98 | C |
| ATOM | 4037 | CE2 | TYR | C | 284 | −8.599 | 14.977 | 15.793 | 1.00 | 51.61 | C |
| ATOM | 4038 | CZ | TYR | C | 284 | −9.289 | 15.524 | 16.868 | 1.00 | 51.68 | C |
| ATOM | 4039 | OH | TYR | C | 284 | −9.263 | 16.890 | 17.083 | 1.00 | 51.99 | O |
| ATOM | 4040 | N | SER | C | 285 | −12.021 | 9.790 | 17.414 | 1.00 | 48.55 | N |
| ATOM | 4041 | CA | SER | C | 285 | −12.840 | 9.893 | 18.618 | 1.00 | 48.39 | C |
| ATOM | 4042 | C | SER | C | 285 | −12.230 | 9.006 | 19.690 | 1.00 | 48.15 | C |
| ATOM | 4043 | O | SER | C | 285 | −11.731 | 7.911 | 19.397 | 1.00 | 48.33 | O |
| ATOM | 4044 | CB | SER | C | 285 | −14.305 | 9.532 | 18.360 | 1.00 | 48.29 | C |
| ATOM | 4045 | OG | SER | C | 285 | −14.472 | 8.137 | 18.247 | 1.00 | 48.63 | O |
| ATOM | 4046 | N | PHE | C | 286 | −12.253 | 9.500 | 20.923 | 1.00 | 47.53 | N |
| ATOM | 4047 | CA | PHE | C | 286 | −11.616 | 8.813 | 22.029 | 1.00 | 46.99 | C |
| ATOM | 4048 | C | PHE | C | 286 | −12.655 | 8.171 | 22.919 | 1.00 | 46.72 | C |
| ATOM | 4049 | O | PHE | C | 286 | −13.658 | 8.789 | 23.263 | 1.00 | 46.75 | O |
| ATOM | 4050 | CB | PHE | C | 286 | −10.713 | 9.768 | 22.825 | 1.00 | 46.73 | C |
| ATOM | 4051 | CG | PHE | C | 286 | −9.463 | 10.154 | 22.095 | 1.00 | 46.43 | C |
| ATOM | 4052 | CD1 | PHE | C | 286 | −8.341 | 9.329 | 22.125 | 1.00 | 45.86 | C |
| ATOM | 4053 | CD2 | PHE | C | 286 | −9.412 | 11.331 | 21.352 | 1.00 | 46.25 | C |
| ATOM | 4054 | CE1 | PHE | C | 286 | −7.178 | 9.679 | 21.439 | 1.00 | 44.56 | C |
| ATOM | 4055 | CE2 | PHE | C | 286 | −8.255 | 11.688 | 20.669 | 1.00 | 45.76 | C |
| ATOM | 4056 | CZ | PHE | C | 286 | −7.135 | 10.855 | 20.716 | 1.00 | 45.39 | C |
| ATOM | 4057 | N | LEU | C | 287 | −12.407 | 6.915 | 23.269 | 1.00 | 46.44 | N |
| ATOM | 4058 | CA | LEU | C | 287 | −13.281 | 6.178 | 24.159 | 1.00 | 46.35 | C |
| ATOM | 4059 | C | LEU | C | 287 | −12.493 | 5.786 | 25.391 | 1.00 | 46.29 | C |
| ATOM | 4060 | O | LEU | C | 287 | −11.456 | 5.117 | 25.294 | 1.00 | 46.52 | O |
| ATOM | 4061 | CB | LEU | C | 287 | −13.863 | 4.944 | 23.459 | 1.00 | 46.32 | C |
| ATOM | 4062 | CG | LEU | C | 287 | −14.604 | 5.174 | 22.134 | 1.00 | 46.58 | C |
| ATOM | 4063 | CD1 | LEU | C | 287 | −15.182 | 3.865 | 21.595 | 1.00 | 46.40 | C |
| ATOM | 4064 | CD2 | LEU | C | 287 | −15.703 | 6.226 | 22.294 | 1.00 | 46.16 | C |
| ATOM | 4065 | N | GLN | C | 288 | −12.984 | 6.225 | 26.546 | 1.00 | 46.00 | N |
| ATOM | 4066 | CA | GLN | C | 288 | −12.346 | 5.939 | 27.824 | 1.00 | 45.53 | C |
| ATOM | 4067 | C | GLN | C | 288 | −13.075 | 4.818 | 28.529 | 1.00 | 45.43 | C |
| ATOM | 4068 | O | GLN | C | 288 | −14.306 | 4.753 | 28.471 | 1.00 | 45.63 | O |
| ATOM | 4069 | CB | GLN | C | 288 | −12.336 | 7.187 | 28.697 | 1.00 | 45.46 | C |
| ATOM | 4070 | CG | GLN | C | 288 | −11.554 | 8.350 | 28.089 | 1.00 | 45.04 | C |
| ATOM | 4071 | CD | GLN | C | 288 | −11.347 | 9.491 | 29.058 | 1.00 | 44.47 | C |
| ATOM | 4072 | OE1 | GLN | C | 288 | −12.181 | 9.761 | 29.915 | 1.00 | 44.75 | O |
| ATOM | 4073 | NE2 | GLN | C | 288 | −10.229 | 10.172 | 28.921 | 1.00 | 45.22 | N |
| ATOM | 4074 | N | PHE | C | 289 | −12.325 | 3.930 | 29.179 | 1.00 | 45.21 | N |
| ATOM | 4075 | CA | PHE | C | 289 | −12.936 | 2.844 | 29.957 | 1.00 | 45.12 | C |
| ATOM | 4076 | C | PHE | C | 289 | −12.241 | 2.500 | 31.284 | 1.00 | 45.14 | C |
| ATOM | 4077 | O | PHE | C | 289 | −11.052 | 2.777 | 31.473 | 1.00 | 45.21 | O |
| ATOM | 4078 | CB | PHE | C | 289 | −13.142 | 1.590 | 29.096 | 1.00 | 44.93 | C |
| ATOM | 4079 | CG | PHE | C | 289 | −11.880 | 1.018 | 28.523 | 1.00 | 44.71 | C |
| ATOM | 4080 | CD1 | PHE | C | 289 | −11.271 | −0.077 | 29.118 | 1.00 | 44.68 | C |
| ATOM | 4081 | CD2 | PHE | C | 289 | −11.314 | 1.553 | 27.367 | 1.00 | 44.98 | C |
| ATOM | 4082 | CE1 | PHE | C | 289 | −10.103 | −0.619 | 28.579 | 1.00 | 45.29 | C |
| ATOM | 4083 | CE2 | PHE | C | 289 | −10.145 | 1.017 | 26.820 | 1.00 | 44.37 | C |
| ATOM | 4084 | CZ | PHE | C | 289 | −9.539 | −0.068 | 27.425 | 1.00 | 44.57 | C |
| ATOM | 4085 | N | ASP | C | 290 | −13.015 | 1.914 | 32.195 | 1.00 | 45.07 | N |
| ATOM | 4086 | CA | ASP | C | 290 | −12.534 | 1.467 | 33.500 | 1.00 | 44.98 | C |
| ATOM | 4087 | C | ASP | C | 290 | −12.089 | 0.006 | 33.424 | 1.00 | 45.32 | C |
| ATOM | 4088 | O | ASP | C | 290 | −12.306 | −0.653 | 32.401 | 1.00 | 45.44 | O |
| ATOM | 4089 | CB | ASP | C | 290 | −13.650 | 1.588 | 34.529 | 1.00 | 44.70 | C |
| ATOM | 4090 | CG | ASP | C | 290 | −14.179 | 2.988 | 34.652 | 1.00 | 44.74 | C |
| ATOM | 4091 | OD1 | ASP | C | 290 | −13.372 | 3.944 | 34.703 | 1.00 | 45.09 | O |
| ATOM | 4092 | OD2 | ASP | C | 290 | −15.414 | 3.129 | 34.710 | 1.00 | 44.88 | O |
| ATOM | 4093 | N | PRO | C | 291 | −11.455 | −0.511 | 34.498 | 1.00 | 45.45 | N |
| ATOM | 4094 | CA | PRO | C | 291 | −11.183 | −1.951 | 34.538 | 1.00 | 45.66 | C |
| ATOM | 4095 | C | PRO | C | 291 | −12.460 | −2.775 | 34.570 | 1.00 | 45.84 | C |
| ATOM | 4096 | O | PRO | C | 291 | −13.493 | −2.285 | 35.032 | 1.00 | 45.93 | O |
| ATOM | 4097 | CB | PRO | C | 291 | −10.417 | −2.131 | 35.854 | 1.00 | 45.57 | C |
| ATOM | 4098 | CG | PRO | C | 291 | −10.737 | −0.929 | 36.659 | 1.00 | 45.17 | C |
| ATOM | 4099 | CD | PRO | C | 291 | −10.925 | 0.178 | 35.687 | 1.00 | 45.17 | C |
| ATOM | 4100 | N | ALA | C | 292 | −12.376 | −4.012 | 34.079 | 1.00 | 46.19 | N |
| ATOM | 4101 | CA | ALA | C | 292 | −13.475 | −4.987 | 34.159 | 1.00 | 46.28 | C |
| ATOM | 4102 | C | ALA | C | 292 | −13.684 | −5.407 | 35.620 | 1.00 | 46.46 | C |
| ATOM | 4103 | O | ALA | C | 292 | −12.734 | −5.395 | 36.395 | 1.00 | 46.71 | O |
| ATOM | 4104 | CB | ALA | C | 292 | −13.156 | −6.188 | 33.292 | 1.00 | 46.23 | C |
| ATOM | 4105 | N | PRO | C | 293 | −14.925 | −5.755 | 36.015 | 1.00 | 46.75 | N |
| ATOM | 4106 | CA | PRO | C | 293 | −15.164 | −6.040 | 37.443 | 1.00 | 46.64 | C |
| ATOM | 4107 | C | PRO | C | 293 | −15.011 | −7.512 | 37.826 | 1.00 | 46.43 | C |
| ATOM | 4108 | O | PRO | C | 293 | −14.729 | −8.347 | 36.969 | 1.00 | 46.45 | O |
| ATOM | 4109 | CB | PRO | C | 293 | −16.614 | −5.585 | 37.652 | 1.00 | 46.51 | C |
| ATOM | 4110 | CG | PRO | C | 293 | −17.252 | −5.615 | 36.261 | 1.00 | 46.87 | C |
| ATOM | 4111 | CD | PRO | C | 293 | −16.166 | −5.890 | 35.229 | 1.00 | 46.90 | C |
| ATOM | 4112 | N | THR | C | 304 | 11.813 | −14.479 | 47.997 | 1.00 | 75.43 | N |
| ATOM | 4113 | CA | THR | C | 304 | 10.966 | −13.337 | 48.319 | 1.00 | 75.55 | C |
| ATOM | 4114 | C | THR | C | 304 | 11.137 | −12.932 | 49.794 | 1.00 | 75.53 | C |
| ATOM | 4115 | O | THR | C | 304 | 10.467 | −13.481 | 50.673 | 1.00 | 75.73 | O |
| ATOM | 4116 | CB | THR | C | 304 | 9.467 | −13.627 | 48.009 | 1.00 | 75.59 | C |
| ATOM | 4117 | OG1 | THR | C | 304 | 9.358 | −14.427 | 46.826 | 1.00 | 75.43 | O |
| ATOM | 4118 | CG2 | THR | C | 304 | 8.686 | −12.325 | 47.815 | 1.00 | 75.68 | C |
| ATOM | 4119 | N | PRO | C | 305 | 12.047 | −11.978 | 50.070 | 1.00 | 75.48 | N |
| ATOM | 4120 | CA | PRO | C | 305 | 12.273 | −11.487 | 51.442 | 1.00 | 75.30 | C |
| ATOM | 4121 | C | PRO | C | 305 | 11.283 | −10.389 | 51.872 | 1.00 | 74.99 | C |
| ATOM | 4122 | O | PRO | C | 305 | 11.127 | −9.385 | 51.166 | 1.00 | 75.02 | O |
| ATOM | 4123 | CB | PRO | C | 305 | 13.707 | −10.925 | 51.389 | 1.00 | 75.34 | C |
| ATOM | 4124 | CG | PRO | C | 305 | 14.149 | −11.017 | 49.927 | 1.00 | 75.48 | C |
| ATOM | 4125 | CD | PRO | C | 305 | 12.938 | −11.316 | 49.103 | 1.00 | 75.42 | C |
| ATOM | 4126 | N | ASP | C | 306 | 10.634 | −10.583 | 53.022 | 1.00 | 74.45 | N |
| ATOM | 4127 | CA | ASP | C | 306 | 9.665 | −9.612 | 53.555 | 1.00 | 74.12 | C |
| ATOM | 4128 | C | ASP | C | 306 | 10.298 | −8.700 | 54.618 | 1.00 | 73.71 | C |
| ATOM | 4129 | O | ASP | C | 306 | 9.786 | −8.558 | 55.735 | 1.00 | 73.62 | O |
| ATOM | 4130 | CB | ASP | C | 306 | 8.436 | −10.339 | 54.118 | 1.00 | 74.29 | C |
| ATOM | 4131 | CG | ASP | C | 306 | 7.229 | −9.421 | 54.290 | 1.00 | 74.72 | C |
| ATOM | 4132 | OD1 | ASP | C | 306 | 6.157 | −9.920 | 54.698 | 1.00 | 74.94 | O |
| ATOM | 4133 | OD2 | ASP | C | 306 | 7.346 | −8.205 | 54.021 | 1.00 | 75.52 | O |
| ATOM | 4134 | N | TYR | C | 307 | 11.409 | −8.074 | 54.245 | 1.00 | 73.15 | N |
| ATOM | 4135 | CA | TYR | C | 307 | 12.243 | −7.318 | 55.167 | 1.00 | 72.65 | C |
| ATOM | 4136 | C | TYR | C | 307 | 12.953 | −6.230 | 54.363 | 1.00 | 72.35 | C |
| ATOM | 4137 | O | TYR | C | 307 | 13.738 | −6.534 | 53.456 | 1.00 | 72.34 | O |
| ATOM | 4138 | CB | TYR | C | 307 | 13.244 | −8.267 | 55.849 | 1.00 | 72.64 | C |
| ATOM | 4139 | CG | TYR | C | 307 | 14.319 | −7.610 | 56.694 | 1.00 | 72.56 | C |
| ATOM | 4140 | CD1 | TYR | C | 307 | 14.106 | −7.352 | 58.048 | 1.00 | 72.24 | C |
| ATOM | 4141 | CD2 | TYR | C | 307 | 15.560 | −7.275 | 56.145 | 1.00 | 72.18 | C |
| ATOM | 4142 | CE1 | TYR | C | 307 | 15.091 | −6.755 | 58.833 | 1.00 | 72.14 | C |
| ATOM | 4143 | CE2 | TYR | C | 307 | 16.553 | −6.680 | 56.920 | 1.00 | 72.38 | C |
| ATOM | 4144 | CZ | TYR | C | 307 | 16.312 | −6.426 | 58.264 | 1.00 | 72.37 | C |
| ATOM | 4145 | OH | TYR | C | 307 | 17.289 | −5.839 | 59.036 | 1.00 | 72.41 | O |
| ATOM | 4146 | N | PHE | C | 308 | 12.662 | −4.972 | 54.690 | 1.00 | 71.83 | N |
| ATOM | 4147 | CA | PHE | C | 308 | 13.178 | −3.830 | 53.933 | 1.00 | 71.48 | C |
| ATOM | 4148 | C | PHE | C | 308 | 13.933 | −2.836 | 54.811 | 1.00 | 71.37 | C |
| ATOM | 4149 | O | PHE | C | 308 | 13.995 | −1.638 | 54.508 | 1.00 | 71.31 | O |
| ATOM | 4150 | CB | PHE | C | 308 | 12.048 | −3.132 | 53.159 | 1.00 | 71.39 | C |
| ATOM | 4151 | CG | PHE | C | 308 | 11.381 | −4.010 | 52.146 | 1.00 | 70.97 | C |
| ATOM | 4152 | CD1 | PHE | C | 308 | 11.989 | −4.262 | 50.921 | 1.00 | 70.87 | C |
| ATOM | 4153 | CD2 | PHE | C | 308 | 10.153 | −4.600 | 52.422 | 1.00 | 70.71 | C |
| ATOM | 4154 | CE1 | PHE | C | 308 | 11.386 | −5.086 | 49.984 | 1.00 | 70.63 | C |
| ATOM | 4155 | CE2 | PHE | C | 308 | 9.538 | −5.429 | 51.488 | 1.00 | 71.06 | C |
| ATOM | 4156 | CZ | PHE | C | 308 | 10.158 | −5.671 | 50.263 | 1.00 | 70.92 | C |
| ATOM | 4157 | N | LEU | C | 309 | 14.527 | −3.348 | 55.884 | 1.00 | 71.18 | N |
| ATOM | 4158 | CA | LEU | C | 309 | 15.213 | −2.520 | 56.865 | 1.00 | 71.06 | C |
| ATOM | 4159 | C | LEU | C | 309 | 16.697 | −2.373 | 56.548 | 1.00 | 71.15 | C |
| ATOM | 4160 | O | LEU | C | 309 | 17.252 | −1.274 | 56.632 | 1.00 | 71.13 | O |
| ATOM | 4161 | CB | LEU | C | 309 | 15.044 | −3.126 | 58.250 | 1.00 | 71.00 | C |
| ATOM | 4162 | CG | LEU | C | 309 | 14.923 | −2.193 | 59.441 | 1.00 | 70.73 | C |
| ATOM | 4163 | CD1 | LEU | C | 309 | 13.569 | −1.507 | 59.417 | 1.00 | 70.54 | C |
| ATOM | 4164 | CD2 | LEU | C | 309 | 15.084 | −3.037 | 60.681 | 1.00 | 70.73 | C |
| ATOM | 4165 | OXT | LEU | C | 309 | 17.375 | −3.344 | 56.199 | 1.00 | 71.19 | O |
| TER | 4166 | | LEU | C | 309 | | | | | | |
| ATOM | 4167 | N | ARG | P | 39 | 35.057 | −22.466 | 51.066 | 1.00 | 48.30 | N |
| ATOM | 4168 | CA | ARG | P | 39 | 35.935 | −23.203 | 52.023 | 1.00 | 48.34 | C |
| ATOM | 4169 | C | ARG | P | 39 | 35.743 | −22.772 | 53.488 | 1.00 | 48.13 | C |
| ATOM | 4170 | O | ARG | P | 39 | 35.101 | −23.474 | 54.283 | 1.00 | 48.16 | O |
| ATOM | 4171 | CB | ARG | P | 39 | 37.410 | −23.065 | 51.610 | 1.00 | 48.36 | C |
| ATOM | 4172 | CG | ARG | P | 39 | 37.958 | −24.240 | 50.822 | 1.00 | 48.67 | C |
| ATOM | 4173 | CD | ARG | P | 39 | 38.107 | −25.480 | 51.697 | 1.00 | 49.05 | C |
| ATOM | 4174 | NE | ARG | P | 39 | 38.958 | −26.498 | 51.080 | 1.00 | 49.37 | N |
| ATOM | 4175 | CZ | ARG | P | 39 | 39.379 | −27.601 | 51.695 | 1.00 | 49.70 | C |
| ATOM | 4176 | NH1 | ARG | P | 39 | 39.029 | −27.846 | 52.954 | 1.00 | 49.82 | N |
| ATOM | 4177 | NH2 | ARG | P | 39 | 40.155 | −28.465 | 51.052 | 1.00 | 49.64 | N |
| ATOM | 4178 | N | ASP | P | 40 | 36.314 | −21.620 | 53.827 | 1.00 | 47.73 | N |
| ATOM | 4179 | CA | ASP | P | 40 | 36.287 | −21.095 | 55.181 | 1.00 | 47.33 | C |
| ATOM | 4180 | C | ASP | P | 40 | 35.018 | −20.297 | 55.424 | 1.00 | 46.73 | C |
| ATOM | 4181 | O | ASP | P | 40 | 34.376 | −19.824 | 54.490 | 1.00 | 46.60 | O |
| ATOM | 4182 | CB | ASP | P | 40 | 37.499 | −20.181 | 55.413 | 1.00 | 47.53 | C |
| ATOM | 4183 | CG | ASP | P | 40 | 38.778 | −20.951 | 55.708 | 1.00 | 48.43 | C |
| ATOM | 4184 | OD1 | ASP | P | 40 | 38.835 | −21.656 | 56.744 | 1.00 | 49.43 | O |
| ATOM | 4185 | OD2 | ASP | P | 40 | 39.741 | −20.832 | 54.915 | 1.00 | 49.04 | O |
| ATOM | 4186 | N | PHE | P | 41 | 34.659 | −20.168 | 56.693 | 1.00 | 46.12 | N |
| ATOM | 4187 | CA | PHE | P | 41 | 33.708 | −19.160 | 57.120 | 1.00 | 45.43 | C |
| ATOM | 4188 | C | PHE | P | 41 | 34.468 | −18.082 | 57.892 | 1.00 | 45.14 | C |
| ATOM | 4189 | O | PHE | P | 41 | 33.871 | −17.172 | 58.464 | 1.00 | 45.28 | O |
| ATOM | 4190 | CB | PHE | P | 41 | 32.586 | −19.785 | 57.962 | 1.00 | 45.28 | C |
| ATOM | 4191 | CG | PHE | P | 41 | 31.603 | −20.600 | 57.157 | 1.00 | 44.96 | C |
| ATOM | 4192 | CD1 | PHE | P | 41 | 30.795 | −19.993 | 56.190 | 1.00 | 44.79 | C |
| ATOM | 4193 | CD2 | PHE | P | 41 | 31.481 | −21.969 | 57.362 | 1.00 | 44.74 | C |
| ATOM | 4194 | CE1 | PHE | P | 41 | 29.884 | −20.741 | 55.439 | 1.00 | 44.45 | C |
| ATOM | 4195 | CE2 | PHE | P | 41 | 30.572 | −22.728 | 56.616 | 1.00 | 44.81 | C |
| ATOM | 4196 | CZ | PHE | P | 41 | 29.772 | −22.111 | 55.652 | 1.00 | 44.63 | C |
| ATOM | 4197 | N | SER | P | 42 | 35.797 | −18.194 | 57.886 | 1.00 | 44.68 | N |
| ATOM | 4198 | CA | SER | P | 42 | 36.687 | −17.247 | 58.558 | 1.00 | 44.16 | C |
| ATOM | 4199 | C | SER | P | 42 | 36.717 | −15.892 | 57.838 | 1.00 | 43.64 | C |
| ATOM | 4200 | O | SER | P | 42 | 36.587 | −15.845 | 56.616 | 1.00 | 43.36 | O |
| ATOM | 4201 | CB | SER | P | 42 | 38.095 | −17.842 | 58.682 | 1.00 | 44.27 | C |
| ATOM | 4202 | OG | SER | P | 42 | 38.628 | −18.175 | 57.412 | 1.00 | 44.46 | O |
| ATOM | 4203 | N | PRO | P | 43 | 36.888 | −14.788 | 58.598 | 1.00 | 43.31 | N |
| ATOM | 4204 | CA | PRO | P | 43 | 36.705 | −13.428 | 58.079 | 1.00 | 43.15 | C |
| ATOM | 4205 | C | PRO | P | 43 | 37.729 | −12.994 | 57.035 | 1.00 | 42.99 | C |
| ATOM | 4206 | O | PRO | P | 43 | 38.736 | −13.668 | 56.823 | 1.00 | 43.21 | O |
| ATOM | 4207 | CB | PRO | P | 43 | 36.838 | −12.548 | 59.333 | 1.00 | 43.04 | C |
| ATOM | 4208 | CG | PRO | P | 43 | 36.695 | −13.470 | 60.483 | 1.00 | 43.15 | C |
| ATOM | 4209 | CD | PRO | P | 43 | 37.267 | −14.762 | 60.019 | 1.00 | 43.28 | C |
| ATOM | 4210 | N | VAL | P | 44 | 37.444 | −11.875 | 56.377 | 1.00 | 42.78 | N |
| ATOM | 4211 | CA | VAL | P | 44 | 38.387 | −11.233 | 55.472 | 1.00 | 42.55 | C |
| ATOM | 4212 | C | VAL | P | 44 | 38.414 | −9.740 | 55.822 | 1.00 | 42.53 | C |
| ATOM | 4213 | O | VAL | P | 44 | 37.365 | −9.094 | 55.842 | 1.00 | 42.42 | O |
| ATOM | 4214 | CB | VAL | P | 44 | 38.021 | −11.477 | 53.981 | 1.00 | 42.44 | C |
| ATOM | 4215 | CG1 | VAL | P | 44 | 38.883 | −10.634 | 53.060 | 1.00 | 42.26 | C |
| ATOM | 4216 | CG2 | VAL | P | 44 | 38.173 | −12.951 | 53.622 | 1.00 | 42.34 | C |
| ATOM | 4217 | N | PRO | P | 45 | 39.611 | −9.197 | 56.135 | 1.00 | 42.54 | N |
| ATOM | 4218 | CA | PRO | P | 45 | 39.750 | −7.781 | 56.506 | 1.00 | 42.48 | C |
| ATOM | 4219 | C | PRO | P | 45 | 39.328 | −6.813 | 55.394 | 1.00 | 42.36 | C |
| ATOM | 4220 | O | PRO | P | 45 | 39.450 | −7.141 | 54.208 | 1.00 | 42.27 | O |
| ATOM | 4221 | CB | PRO | P | 45 | 41.255 | −7.639 | 56.796 | 1.00 | 42.48 | C |
| ATOM | 4222 | CG | PRO | P | 45 | 41.901 | −8.782 | 56.089 | 1.00 | 42.34 | C |
| ATOM | 4223 | CD | PRO | P | 45 | 40.910 | −9.898 | 56.168 | 1.00 | 42.52 | C |
| ATOM | 4224 | N | TRP | P | 46 | 38.849 | −5.634 | 55.790 | 1.00 | 42.22 | N |
| ATOM | 4225 | CA | TRP | P | 46 | 38.412 | −4.592 | 54.854 | 1.00 | 42.30 | C |
| ATOM | 4226 | C | TRP | P | 46 | 39.515 | −4.158 | 53.888 | 1.00 | 42.36 | C |
| ATOM | 4227 | O | TRP | P | 46 | 39.238 | −3.817 | 52.734 | 1.00 | 42.33 | O |
| ATOM | 4228 | CB | TRP | P | 46 | 37.868 | −3.372 | 55.609 | 1.00 | 42.30 | C |
| ATOM | 4229 | CG | TRP | P | 46 | 38.881 | −2.711 | 56.483 | 1.00 | 42.29 | C |
| ATOM | 4230 | CD1 | TRP | P | 46 | 39.095 | −2.949 | 57.805 | 1.00 | 42.23 | C |
| ATOM | 4231 | CD2 | TRP | P | 46 | 39.833 | −1.710 | 56.092 | 1.00 | 42.52 | C |
| ATOM | 4232 | NE1 | TRP | P | 46 | 40.121 | −2.161 | 58.267 | 1.00 | 42.57 | N |
| ATOM | 4233 | CE2 | TRP | P | 46 | 40.591 | −1.390 | 57.237 | 1.00 | 42.65 | C |
| ATOM | 4234 | CE3 | TRP | P | 46 | 40.117 | −1.053 | 54.886 | 1.00 | 42.34 | C |
| ATOM | 4235 | CZ2 | TRP | P | 46 | 41.620 | −0.440 | 57.213 | 1.00 | 42.59 | C |
| ATOM | 4236 | CZ3 | TRP | P | 46 | 41.141 | −0.114 | 54.862 | 1.00 | 42.37 | C |
| ATOM | 4237 | CH2 | TRP | P | 46 | 41.878 | 0.185 | 56.019 | 1.00 | 42.34 | C |
| ATOM | 4238 | N | SER | P | 47 | 40.758 | −4.185 | 54.373 | 1.00 | 42.47 | N |
| ATOM | 4239 | CA | SER | P | 47 | 41.939 | −3.724 | 53.630 | 1.00 | 42.53 | C |
| ATOM | 4240 | C | SER | P | 47 | 42.213 | −4.531 | 52.366 | 1.00 | 42.50 | C |
| ATOM | 4241 | O | SER | P | 47 | 43.159 | −4.240 | 51.631 | 1.00 | 42.61 | O |
| ATOM | 4242 | CB | SER | P | 47 | 43.168 | −3.743 | 54.537 | 1.00 | 42.44 | C |
| ATOM | 4243 | OG | SER | P | 47 | 43.283 | −4.995 | 55.191 | 1.00 | 42.79 | O |
| ATOM | 4244 | N | GLN | P | 48 | 41.384 | −5.544 | 52.129 | 1.00 | 42.44 | N |
| ATOM | 4245 | CA | GLN | P | 48 | 41.417 | −6.322 | 50.896 | 1.00 | 42.39 | C |
| ATOM | 4246 | C | GLN | P | 48 | 40.623 | −5.644 | 49.783 | 1.00 | 42.21 | C |
| ATOM | 4247 | O | GLN | P | 48 | 40.785 | −5.974 | 48.609 | 1.00 | 42.24 | O |
| ATOM | 4248 | CB | GLN | P | 48 | 40.860 | −7.729 | 51.139 | 1.00 | 42.37 | C |
| ATOM | 4249 | CG | GLN | P | 48 | 41.859 | −8.712 | 51.752 | 1.00 | 42.87 | C |
| ATOM | 4250 | CD | GLN | P | 48 | 42.962 | −9.145 | 50.783 | 1.00 | 42.89 | C |
| ATOM | 4251 | OE1 | GLN | P | 48 | 44.049 | −9.538 | 51.207 | 1.00 | 43.03 | O |
| ATOM | 4252 | NE2 | GLN | P | 48 | 42.683 | −9.078 | 49.482 | 1.00 | 42.37 | N |
| ATOM | 4253 | N | TYR | P | 49 | 39.768 | −4.697 | 50.157 | 1.00 | 42.12 | N |
| ATOM | 4254 | CA | TYR | P | 49 | 38.826 | −4.098 | 49.217 | 1.00 | 41.96 | C |
| ATOM | 4255 | C | TYR | P | 49 | 38.897 | −2.578 | 49.196 | 1.00 | 42.26 | C |
| ATOM | 4256 | O | TYR | P | 49 | 38.676 | −1.964 | 48.154 | 1.00 | 42.33 | O |
| ATOM | 4257 | CB | TYR | P | 49 | 37.401 | −4.558 | 49.532 | 1.00 | 41.61 | C |
| ATOM | 4258 | CG | TYR | P | 49 | 37.168 | −6.040 | 49.326 | 1.00 | 40.82 | C |
| ATOM | 4259 | CD1 | TYR | P | 49 | 36.842 | −6.545 | 48.067 | 1.00 | 40.49 | C |
| ATOM | 4260 | CD2 | TYR | P | 49 | 37.270 | −6.935 | 50.388 | 1.00 | 40.04 | C |
| ATOM | 4261 | CE1 | TYR | P | 49 | 36.627 | −7.901 | 47.873 | 1.00 | 40.40 | C |
| ATOM | 4262 | CE2 | TYR | P | 49 | 37.057 | −8.295 | 50.204 | 1.00 | 40.00 | C |
| ATOM | 4263 | CZ | TYR | P | 49 | 36.737 | −8.770 | 48.945 | 1.00 | 40.32 | C |
| ATOM | 4264 | OH | TYR | P | 49 | 36.523 | −10.111 | 48.757 | 1.00 | 40.19 | O |
| ATOM | 4265 | N | PHE | P | 50 | 39.199 | −1.980 | 50.345 | 1.00 | 42.68 | N |
| ATOM | 4266 | CA | PHE | P | 50 | 39.362 | −0.533 | 50.450 | 1.00 | 43.16 | C |
| ATOM | 4267 | C | PHE | P | 50 | 40.832 | −0.134 | 50.542 | 1.00 | 43.71 | C |
| ATOM | 4268 | O | PHE | P | 50 | 41.627 | −0.789 | 51.230 | 1.00 | 43.82 | O |
| ATOM | 4269 | CB | PHE | P | 50 | 38.608 | 0.011 | 51.663 | 1.00 | 43.08 | C |
| ATOM | 4270 | CG | PHE | P | 50 | 37.116 | −0.103 | 51.555 | 1.00 | 43.16 | C |
| ATOM | 4271 | CD1 | PHE | P | 50 | 36.396 | 0.753 | 50.722 | 1.00 | 42.79 | C |
| ATOM | 4272 | CD2 | PHE | P | 50 | 36.423 | −1.057 | 52.296 | 1.00 | 43.14 | C |
| ATOM | 4273 | CE1 | PHE | P | 50 | 35.014 | 0.653 | 50.622 | 1.00 | 42.72 | C |
| ATOM | 4274 | CE2 | PHE | P | 50 | 35.031 | −1.160 | 52.203 | 1.00 | 42.96 | C |
| ATOM | 4275 | CZ | PHE | P | 50 | 34.329 | −0.303 | 51.365 | 1.00 | 42.67 | C |
| ATOM | 4276 | N | GLU | P | 51 | 41.179 | 0.950 | 49.851 | 1.00 | 44.22 | N |
| ATOM | 4277 | CA | GLU | P | 51 | 42.533 | 1.498 | 49.868 | 1.00 | 44.72 | C |
| ATOM | 4278 | C | GLU | P | 51 | 42.890 | 2.156 | 51.204 | 1.00 | 45.31 | C |
| ATOM | 4279 | O | GLU | P | 51 | 44.049 | 2.119 | 51.627 | 1.00 | 45.34 | O |
| ATOM | 4280 | CB | GLU | P | 51 | 42.718 | 2.502 | 48.728 | 1.00 | 44.60 | C |
| ATOM | 4281 | CG | GLU | P | 51 | 42.791 | 1.871 | 47.349 | 1.00 | 44.29 | C |
| ATOM | 4282 | CD | GLU | P | 51 | 42.460 | 2.851 | 46.239 | 1.00 | 43.92 | C |
| ATOM | 4283 | OE1 | GLU | P | 51 | 43.121 | 3.905 | 46.146 | 1.00 | 44.58 | O |
| ATOM | 4284 | OE2 | GLU | P | 51 | 41.539 | 2.565 | 45.451 | 1.00 | 43.15 | O |
| ATOM | 4285 | N | SER | P | 52 | 41.899 | 2.761 | 51.859 | 1.00 | 46.00 | N |
| ATOM | 4286 | CA | SER | P | 52 | 42.121 | 3.453 | 53.129 | 1.00 | 46.69 | C |
| ATOM | 4287 | C | SER | P | 52 | 40.850 | 3.597 | 53.968 | 1.00 | 47.24 | C |
| ATOM | 4288 | O | SER | P | 52 | 39.781 | 3.112 | 53.593 | 1.00 | 47.24 | O |
| ATOM | 4289 | CB | SER | P | 52 | 42.735 | 4.833 | 52.880 | 1.00 | 46.56 | C |
| ATOM | 4290 | OG | SER | P | 52 | 41.770 | 5.721 | 52.352 | 1.00 | 46.91 | O |
| ATOM | 4291 | N | MET | P | 53 | 40.997 | 4.256 | 55.116 | 1.00 | 48.09 | N |
| ATOM | 4292 | CA | MET | P | 53 | 39.882 | 4.619 | 55.994 | 1.00 | 48.96 | C |
| ATOM | 4293 | C | MET | P | 53 | 40.167 | 5.937 | 56.729 | 1.00 | 49.16 | C |
| ATOM | 4294 | O | MET | P | 53 | 41.304 | 6.423 | 56.739 | 1.00 | 49.20 | O |
| ATOM | 4295 | CB | MET | P | 53 | 39.560 | 3.496 | 56.996 | 1.00 | 48.81 | C |
| ATOM | 4296 | CG | MET | P | 53 | 40.710 | 3.079 | 57.901 | 1.00 | 49.21 | C |
| ATOM | 4297 | SD | MET | P | 53 | 40.168 | 2.600 | 59.559 | 1.00 | 49.86 | S |
| ATOM | 4298 | CE | MET | P | 53 | 40.401 | 4.142 | 60.453 | 1.00 | 49.21 | C |
| ATOM | 4299 | N | GLU | P | 54 | 39.126 | 6.513 | 57.328 | 1.00 | 49.57 | N |
| ATOM | 4300 | CA | GLU | P | 54 | 39.253 | 7.741 | 58.109 | 1.00 | 49.92 | C |
| ATOM | 4301 | C | GLU | P | 54 | 38.162 | 7.822 | 59.171 | 1.00 | 50.23 | C |
| ATOM | 4302 | O | GLU | P | 54 | 37.010 | 7.458 | 58.920 | 1.00 | 50.16 | O |
| ATOM | 4303 | CB | GLU | P | 54 | 39.198 | 8.975 | 57.200 | 1.00 | 49.79 | C |
| ATOM | 4304 | CG | GLU | P | 54 | 39.464 | 10.294 | 57.918 | 1.00 | 49.80 | C |
| ATOM | 4305 | CD | GLU | P | 54 | 39.464 | 11.500 | 56.991 | 1.00 | 50.08 | C |
| ATOM | 4306 | OE1 | GLU | P | 54 | 39.192 | 11.338 | 55.780 | 1.00 | 50.34 | O |
| ATOM | 4307 | OE2 | GLU | P | 54 | 39.740 | 12.618 | 57.482 | 1.00 | 50.07 | O |
| ATOM | 4308 | N | ASP | P | 55 | 38.543 | 8.294 | 60.357 | 1.00 | 50.61 | N |
| ATOM | 4309 | CA | ASP | P | 55 | 37.600 | 8.588 | 61.433 | 1.00 | 50.97 | C |
| ATOM | 4310 | C | ASP | P | 55 | 37.289 | 10.088 | 61.456 | 1.00 | 51.10 | C |
| ATOM | 4311 | O | ASP | P | 55 | 37.953 | 10.861 | 62.146 | 1.00 | 51.21 | O |
| ATOM | 4312 | CB | ASP | P | 55 | 38.164 | 8.128 | 62.781 | 1.00 | 51.00 | C |
| ATOM | 4313 | CG | ASP | P | 55 | 37.903 | 6.655 | 63.061 | 1.00 | 51.40 | C |
| ATOM | 4314 | OD1 | ASP | P | 55 | 36.720 | 6.253 | 63.194 | 1.00 | 51.67 | O |
| ATOM | 4315 | OD2 | ASP | P | 55 | 38.892 | 5.900 | 63.170 | 1.00 | 51.54 | O |
| ATOM | 4316 | N | VAL | P | 56 | 36.268 | 10.480 | 60.698 | 1.00 | 51.30 | N |
| ATOM | 4317 | CA | VAL | P | 56 | 35.936 | 11.886 | 60.462 | 1.00 | 51.53 | C |
| ATOM | 4318 | C | VAL | P | 56 | 35.292 | 12.572 | 61.672 | 1.00 | 51.83 | C |
| ATOM | 4319 | O | VAL | P | 56 | 34.317 | 12.073 | 62.236 | 1.00 | 51.74 | O |
| ATOM | 4320 | CB | VAL | P | 56 | 35.033 | 12.026 | 59.212 | 1.00 | 51.47 | C |
| ATOM | 4321 | CG1 | VAL | P | 56 | 34.579 | 13.463 | 59.012 | 1.00 | 51.45 | C |
| ATOM | 4322 | CG2 | VAL | P | 56 | 35.767 | 11.527 | 57.979 | 1.00 | 51.54 | C |
| ATOM | 4323 | N | GLU | P | 57 | 35.842 | 13.726 | 62.046 | 1.00 | 52.35 | N |
| ATOM | 4324 | CA | GLU | P | 57 | 35.389 | 14.482 | 63.218 | 1.00 | 52.93 | C |
| ATOM | 4325 | C | GLU | P | 57 | 34.301 | 15.500 | 62.870 | 1.00 | 53.36 | C |
| ATOM | 4326 | O | GLU | P | 57 | 34.443 | 16.265 | 61.915 | 1.00 | 53.46 | O |
| ATOM | 4327 | CB | GLU | P | 57 | 36.572 | 15.184 | 63.889 | 1.00 | 52.83 | C |
| ATOM | 4328 | CG | GLU | P | 57 | 37.717 | 14.256 | 64.268 | 1.00 | 52.76 | C |
| ATOM | 4329 | CD | GLU | P | 57 | 38.914 | 14.994 | 64.833 | 1.00 | 53.00 | C |
| ATOM | 4330 | OE1 | GLU | P | 57 | 39.319 | 16.031 | 64.260 | 1.00 | 52.82 | O |
| ATOM | 4331 | OE2 | GLU | P | 57 | 39.460 | 14.526 | 65.853 | 1.00 | 53.57 | O |
| ATOM | 4332 | N | VAL | P | 58 | 33.223 | 15.504 | 63.656 | 1.00 | 53.93 | N |
| ATOM | 4333 | CA | VAL | P | 58 | 32.055 | 16.364 | 63.410 | 1.00 | 54.44 | C |
| ATOM | 4334 | C | VAL | P | 58 | 31.515 | 16.951 | 64.721 | 1.00 | 54.87 | C |
| ATOM | 4335 | O | VAL | P | 58 | 31.272 | 16.217 | 65.681 | 1.00 | 54.87 | O |
| ATOM | 4336 | CB | VAL | P | 58 | 30.923 | 15.586 | 62.668 | 1.00 | 54.43 | C |
| ATOM | 4337 | CG1 | VAL | P | 58 | 29.613 | 16.373 | 62.665 | 1.00 | 54.33 | C |
| ATOM | 4338 | CG2 | VAL | P | 58 | 31.341 | 15.236 | 61.243 | 1.00 | 54.38 | C |
| ATOM | 4339 | N | GLU | P | 59 | 31.328 | 18.273 | 64.745 | 1.00 | 55.42 | N |
| ATOM | 4340 | CA | GLU | P | 59 | 30.838 | 18.992 | 65.932 | 1.00 | 55.97 | C |
| ATOM | 4341 | C | GLU | P | 59 | 29.403 | 19.511 | 65.755 | 1.00 | 56.27 | C |
| ATOM | 4342 | O | GLU | P | 59 | 28.995 | 19.858 | 64.642 | 1.00 | 56.42 | O |
| ATOM | 4343 | CB | GLU | P | 59 | 31.785 | 20.151 | 66.296 | 1.00 | 55.84 | C |
| ATOM | 4344 | CG | GLU | P | 59 | 31.930 | 21.239 | 65.212 | 1.00 | 55.94 | C |
| ATOM | 4345 | CD | GLU | P | 59 | 32.858 | 22.393 | 65.622 | 1.00 | 56.25 | C |
| ATOM | 4346 | OE1 | GLU | P | 59 | 33.001 | 22.682 | 66.874 | 1.00 | 56.40 | O |
| ATOM | 4347 | OE2 | GLU | P | 59 | 33.442 | 23.023 | 64.681 | 1.00 | 56.35 | O |
| ATOM | 4348 | N | ASN | P | 60 | 28.648 | 19.554 | 66.854 | 1.00 | 56.62 | N |
| ATOM | 4349 | CA | ASN | P | 60 | 27.288 | 20.129 | 66.869 | 1.00 | 56.80 | C |
| ATOM | 4350 | C | ASN | P | 60 | 26.969 | 20.960 | 68.126 | 1.00 | 56.95 | C |
| ATOM | 4351 | O | ASN | P | 60 | 27.634 | 21.969 | 68.385 | 1.00 | 56.93 | O |
| ATOM | 4352 | CB | ASN | P | 60 | 26.204 | 19.066 | 66.597 | 1.00 | 56.79 | C |
| ATOM | 4353 | CG | ASN | P | 60 | 26.529 | 17.701 | 67.202 | 1.00 | 56.95 | C |
| ATOM | 4354 | OD1 | ASN | P | 60 | 26.439 | 16.681 | 66.517 | 1.00 | 57.22 | O |
| ATOM | 4355 | ND2 | ASN | P | 60 | 26.893 | 17.675 | 68.485 | 1.00 | 57.10 | N |
| ATOM | 4356 | N | GLU | P | 61 | 25.962 | 20.537 | 68.895 | 1.00 | 57.11 | N |
| ATOM | 4357 | CA | GLU | P | 61 | 25.507 | 21.281 | 70.079 | 1.00 | 57.22 | C |
| ATOM | 4358 | C | GLU | P | 61 | 26.034 | 20.725 | 71.408 | 1.00 | 57.04 | C |
| ATOM | 4359 | O | GLU | P | 61 | 26.678 | 21.447 | 72.178 | 1.00 | 56.87 | O |
| ATOM | 4360 | CB | GLU | P | 61 | 23.975 | 21.381 | 70.099 | 1.00 | 57.36 | C |
| ATOM | 4361 | CG | GLU | P | 61 | 23.411 | 22.487 | 69.209 | 1.00 | 57.87 | C |
| ATOM | 4362 | CD | GLU | P | 61 | 22.076 | 22.121 | 68.568 | 1.00 | 58.41 | C |
| ATOM | 4363 | OE1 | GLU | P | 61 | 21.247 | 23.034 | 68.362 | 1.00 | 58.75 | O |
| ATOM | 4364 | OE2 | GLU | P | 61 | 21.856 | 20.927 | 68.262 | 1.00 | 58.41 | O |
| ATOM | 4365 | N | THR | P | 62 | 25.757 | 19.447 | 71.670 | 1.00 | 56.85 | N |
| ATOM | 4366 | CA | THR | P | 62 | 26.211 | 18.789 | 72.901 | 1.00 | 56.60 | C |
| ATOM | 4367 | C | THR | P | 62 | 27.712 | 18.465 | 72.875 | 1.00 | 56.21 | C |
| ATOM | 4368 | O | THR | P | 62 | 28.400 | 18.629 | 73.889 | 1.00 | 56.06 | O |
| ATOM | 4369 | CB | THR | P | 62 | 25.386 | 17.508 | 73.233 | 1.00 | 56.72 | C |
| ATOM | 4370 | OG1 | THR | P | 62 | 25.131 | 16.761 | 72.033 | 1.00 | 56.88 | O |
| ATOM | 4371 | CG2 | THR | P | 62 | 24.056 | 17.877 | 73.901 | 1.00 | 56.76 | C |
| ATOM | 4372 | N | GLY | P | 63 | 28.212 | 18.016 | 71.719 | 1.00 | 55.74 | N |
| ATOM | 4373 | CA | GLY | P | 63 | 29.633 | 17.692 | 71.579 | 1.00 | 55.02 | C |
| ATOM | 4374 | C | GLY | P | 63 | 30.132 | 17.347 | 70.184 | 1.00 | 54.45 | C |
| ATOM | 4375 | O | GLY | P | 63 | 29.722 | 17.954 | 69.190 | 1.00 | 54.31 | O |
| ATOM | 4376 | N | LYS | P | 64 | 31.023 | 16.356 | 70.137 | 1.00 | 53.89 | N |
| ATOM | 4377 | CA | LYS | P | 64 | 31.779 | 15.984 | 68.940 | 1.00 | 53.19 | C |
| ATOM | 4378 | C | LYS | P | 64 | 31.645 | 14.480 | 68.645 | 1.00 | 52.60 | C |
| ATOM | 4379 | O | LYS | P | 64 | 31.625 | 13.657 | 69.563 | 1.00 | 52.53 | O |
| ATOM | 4380 | CB | LYS | P | 64 | 33.254 | 16.401 | 69.123 | 1.00 | 53.26 | C |
| ATOM | 4381 | CG | LYS | P | 64 | 34.263 | 15.857 | 68.104 | 1.00 | 53.35 | C |
| ATOM | 4382 | CD | LYS | P | 64 | 34.850 | 14.515 | 68.552 | 1.00 | 53.26 | C |
| ATOM | 4383 | CE | LYS | P | 64 | 35.872 | 13.975 | 67.564 | 1.00 | 53.14 | C |
| ATOM | 4384 | NZ | LYS | P | 64 | 37.187 | 14.660 | 67.679 | 1.00 | 52.49 | N |
| ATOM | 4385 | N | ASP | P | 65 | 31.559 | 14.135 | 67.360 | 1.00 | 51.87 | N |
| ATOM | 4386 | CA | ASP | P | 65 | 31.389 | 12.745 | 66.933 | 1.00 | 51.15 | C |
| ATOM | 4387 | C | ASP | P | 65 | 32.444 | 12.304 | 65.920 | 1.00 | 50.60 | C |
| ATOM | 4388 | O | ASP | P | 65 | 32.965 | 13.118 | 65.156 | 1.00 | 50.64 | O |
| ATOM | 4389 | CB | ASP | P | 65 | 29.992 | 12.531 | 66.344 | 1.00 | 51.18 | C |
| ATOM | 4390 | CG | ASP | P | 65 | 28.880 | 12.975 | 67.281 | 1.00 | 51.35 | C |
| ATOM | 4391 | OD1 | ASP | P | 65 | 28.959 | 12.704 | 68.500 | 1.00 | 51.36 | O |
| ATOM | 4392 | OD2 | ASP | P | 65 | 27.913 | 13.591 | 66.786 | 1.00 | 51.61 | O |
| ATOM | 4393 | N | THR | P | 66 | 32.742 | 11.006 | 65.924 | 1.00 | 49.87 | N |
| ATOM | 4394 | CA | THR | P | 66 | 33.682 | 10.395 | 64.985 | 1.00 | 49.22 | C |
| ATOM | 4395 | C | THR | P | 66 | 32.952 | 9.346 | 64.146 | 1.00 | 48.81 | C |
| ATOM | 4396 | O | THR | P | 66 | 32.225 | 8.506 | 64.685 | 1.00 | 48.81 | O |
| ATOM | 4397 | CB | THR | P | 66 | 34.860 | 9.748 | 65.722 | 1.00 | 49.20 | C |
| ATOM | 4398 | OG1 | THR | P | 66 | 35.482 | 10.723 | 66.566 | 1.00 | 49.52 | O |
| ATOM | 4399 | CG2 | THR | P | 66 | 35.881 | 9.226 | 64.741 | 1.00 | 48.90 | C |
| ATOM | 4400 | N | PHE | P | 67 | 33.148 | 9.387 | 62.831 | 1.00 | 48.10 | N |
| ATOM | 4401 | CA | PHE | P | 67 | 32.250 | 8.660 | 61.948 | 1.00 | 47.45 | C |
| ATOM | 4402 | C | PHE | P | 67 | 32.758 | 7.367 | 61.308 | 1.00 | 47.30 | C |
| ATOM | 4403 | O | PHE | P | 67 | 32.061 | 6.350 | 61.345 | 1.00 | 47.33 | O |
| ATOM | 4404 | CB | PHE | P | 67 | 31.591 | 9.601 | 60.938 | 1.00 | 47.26 | C |
| ATOM | 4405 | CG | PHE | P | 67 | 30.327 | 10.228 | 61.456 | 1.00 | 46.78 | C |
| ATOM | 4406 | CD1 | PHE | P | 67 | 29.089 | 9.678 | 61.150 | 1.00 | 46.47 | C |
| ATOM | 4407 | CD2 | PHE | P | 67 | 30.374 | 11.348 | 62.278 | 1.00 | 46.80 | C |
| ATOM | 4408 | CE1 | PHE | P | 67 | 27.915 | 10.242 | 61.639 | 1.00 | 46.38 | C |
| ATOM | 4409 | CE2 | PHE | P | 67 | 29.201 | 11.924 | 62.771 | 1.00 | 46.72 | C |
| ATOM | 4410 | CZ | PHE | P | 67 | 27.971 | 11.368 | 62.450 | 1.00 | 46.50 | C |
| ATOM | 4411 | N | ARG | P | 68 | 33.957 | 7.394 | 60.739 | 1.00 | 46.83 | N |
| ATOM | 4412 | CA | ARG | P | 68 | 34.500 | 6.212 | 60.049 | 1.00 | 46.61 | C |
| ATOM | 4413 | C | ARG | P | 68 | 33.959 | 6.088 | 58.634 | 1.00 | 46.23 | C |
| ATOM | 4414 | O | ARG | P | 68 | 32.770 | 5.841 | 58.428 | 1.00 | 46.06 | O |
| ATOM | 4415 | CB | ARG | P | 68 | 34.240 | 4.902 | 60.825 | 1.00 | 46.49 | C |
| ATOM | 4416 | CG | ARG | P | 68 | 34.753 | 3.619 | 60.143 | 1.00 | 46.41 | C |
| ATOM | 4417 | CD | ARG | P | 68 | 36.282 | 3.522 | 60.156 | 1.00 | 45.24 | C |
| ATOM | 4418 | NE | ARG | P | 68 | 36.839 | 3.722 | 61.496 | 1.00 | 44.73 | N |
| ATOM | 4419 | CZ | ARG | P | 68 | 37.269 | 2.751 | 62.300 | 1.00 | 44.23 | C |
| ATOM | 4420 | NH1 | ARG | P | 68 | 37.225 | 1.480 | 61.923 | 1.00 | 44.12 | N |
| ATOM | 4421 | NH2 | ARG | P | 68 | 37.751 | 3.056 | 63.492 | 1.00 | 44.08 | N |
| ATOM | 4422 | N | VAL | P | 69 | 34.854 | 6.265 | 57.671 | 1.00 | 45.89 | N |
| ATOM | 4423 | CA | VAL | P | 69 | 34.549 | 6.035 | 56.271 | 1.00 | 45.63 | C |
| ATOM | 4424 | C | VAL | P | 69 | 35.614 | 5.129 | 55.677 | 1.00 | 45.49 | C |
| ATOM | 4425 | O | VAL | P | 69 | 36.774 | 5.177 | 56.084 | 1.00 | 45.34 | O |
| ATOM | 4426 | CB | VAL | P | 69 | 34.462 | 7.357 | 55.460 | 1.00 | 45.66 | C |
| ATOM | 4427 | CG1 | VAL | P | 69 | 33.137 | 8.056 | 55.716 | 1.00 | 45.73 | C |
| ATOM | 4428 | CG2 | VAL | P | 69 | 35.635 | 8.282 | 55.775 | 1.00 | 45.47 | C |
| ATOM | 4429 | N | TYR | P | 70 | 35.210 | 4.295 | 54.726 | 1.00 | 45.35 | N |
| ATOM | 4430 | CA | TYR | P | 70 | 36.149 | 3.462 | 53.985 | 1.00 | 45.23 | C |
| ATOM | 4431 | C | TYR | P | 70 | 36.244 | 3.958 | 52.546 | 1.00 | 45.02 | C |
| ATOM | 4432 | O | TYR | P | 70 | 35.237 | 4.043 | 51.840 | 1.00 | 44.92 | O |
| ATOM | 4433 | CB | TYR | P | 70 | 35.738 | 1.992 | 54.048 | 1.00 | 45.26 | C |
| ATOM | 4434 | CG | TYR | P | 70 | 35.752 | 1.410 | 55.445 | 1.00 | 45.39 | C |
| ATOM | 4435 | CD1 | TYR | P | 70 | 36.885 | 0.765 | 55.945 | 1.00 | 45.45 | C |
| ATOM | 4436 | CD2 | TYR | P | 70 | 34.627 | 1.499 | 56.268 | 1.00 | 45.56 | C |
| ATOM | 4437 | CE1 | TYR | P | 70 | 36.897 | 0.223 | 57.230 | 1.00 | 45.53 | C |
| ATOM | 4438 | CE2 | TYR | P | 70 | 34.626 | 0.963 | 57.548 | 1.00 | 45.62 | C |
| ATOM | 4439 | CZ | TYR | P | 70 | 35.761 | 0.326 | 58.025 | 1.00 | 45.67 | C |
| ATOM | 4440 | OH | TYR | P | 70 | 35.755 | −0.198 | 59.299 | 1.00 | 45.48 | O |
| ATOM | 4441 | N | LYS | P | 71 | 37.465 | 4.295 | 52.137 | 1.00 | 44.88 | N |
| ATOM | 4442 | CA | LYS | P | 71 | 37.726 | 4.977 | 50.868 | 1.00 | 44.75 | C |
| ATOM | 4443 | C | LYS | P | 71 | 38.448 | 4.095 | 49.853 | 1.00 | 44.89 | C |
| ATOM | 4444 | O | LYS | P | 71 | 39.109 | 3.117 | 50.215 | 1.00 | 45.01 | O |
| ATOM | 4445 | CB | LYS | P | 71 | 38.550 | 6.245 | 51.108 | 1.00 | 44.56 | C |
| ATOM | 4446 | CG | LYS | P | 71 | 37.800 | 7.360 | 51.816 | 1.00 | 44.37 | C |
| ATOM | 4447 | CD | LYS | P | 71 | 38.705 | 8.556 | 52.097 | 1.00 | 44.49 | C |
| ATOM | 4448 | CE | LYS | P | 71 | 39.656 | 8.290 | 53.259 | 1.00 | 44.18 | C |
| ATOM | 4449 | NZ | LYS | P | 71 | 40.519 | 9.461 | 53.556 | 1.00 | 43.78 | N |
| ATOM | 4450 | N | SER | P | 72 | 38.317 | 4.459 | 48.580 | 1.00 | 44.87 | N |
| ATOM | 4451 | CA | SER | P | 72 | 39.029 | 3.794 | 47.496 | 1.00 | 44.87 | C |
| ATOM | 4452 | C | SER | P | 72 | 38.915 | 4.628 | 46.224 | 1.00 | 44.95 | C |
| ATOM | 4453 | O | SER | P | 72 | 37.829 | 5.095 | 45.879 | 1.00 | 44.95 | O |
| ATOM | 4454 | CB | SER | P | 72 | 38.466 | 2.391 | 47.271 | 1.00 | 44.85 | C |
| ATOM | 4455 | OG | SER | P | 72 | 39.202 | 1.693 | 46.291 | 1.00 | 45.05 | O |
| ATOM | 4456 | N | GLY | P | 73 | 40.041 | 4.829 | 45.542 | 1.00 | 45.13 | N |
| ATOM | 4457 | CA | GLY | P | 73 | 40.067 | 5.571 | 44.274 | 1.00 | 45.16 | C |
| ATOM | 4458 | C | GLY | P | 73 | 40.625 | 6.972 | 44.418 | 1.00 | 45.09 | C |
| ATOM | 4459 | O | GLY | P | 73 | 40.649 | 7.520 | 45.519 | 1.00 | 45.05 | O |
| ATOM | 4460 | N | SER | P | 74 | 41.053 | 7.557 | 43.301 | 1.00 | 45.19 | N |
| ATOM | 4461 | CA | SER | P | 74 | 41.761 | 8.839 | 43.330 | 1.00 | 45.38 | C |
| ATOM | 4462 | C | SER | P | 74 | 41.204 | 9.920 | 42.399 | 1.00 | 45.54 | C |
| ATOM | 4463 | O | SER | P | 74 | 41.464 | 11.108 | 42.611 | 1.00 | 45.64 | O |
| ATOM | 4464 | CB | SER | P | 74 | 43.256 | 8.629 | 43.058 | 1.00 | 45.32 | C |
| ATOM | 4465 | OG | SER | P | 74 | 43.464 | 8.031 | 41.792 | 1.00 | 45.26 | O |
| ATOM | 4466 | N | GLU | P | 75 | 40.460 | 9.513 | 41.372 | 1.00 | 45.63 | N |
| ATOM | 4467 | CA | GLU | P | 75 | 39.867 | 10.451 | 40.406 | 1.00 | 45.90 | C |
| ATOM | 4468 | C | GLU | P | 75 | 38.403 | 10.112 | 40.113 | 1.00 | 45.94 | C |
| ATOM | 4469 | O | GLU | P | 75 | 37.869 | 9.134 | 40.647 | 1.00 | 46.07 | O |
| ATOM | 4470 | CB | GLU | P | 75 | 40.678 | 10.476 | 39.107 | 1.00 | 45.94 | C |
| ATOM | 4471 | CG | GLU | P | 75 | 42.016 | 11.194 | 39.218 | 1.00 | 46.94 | C |
| ATOM | 4472 | CD | GLU | P | 75 | 42.999 | 10.784 | 38.133 | 1.00 | 48.40 | C |
| ATOM | 4473 | OE1 | GLU | P | 75 | 42.577 | 10.130 | 37.152 | 1.00 | 49.26 | O |
| ATOM | 4474 | OE2 | GLU | P | 75 | 44.200 | 11.115 | 38.263 | 1.00 | 48.83 | O |
| ATOM | 4475 | N | GLY | P | 76 | 37.760 | 10.922 | 39.269 | 1.00 | 45.86 | N |
| ATOM | 4476 | CA | GLY | P | 76 | 36.339 | 10.756 | 38.939 | 1.00 | 45.50 | C |
| ATOM | 4477 | C | GLY | P | 76 | 35.413 | 11.161 | 40.075 | 1.00 | 45.36 | C |
| ATOM | 4478 | O | GLY | P | 76 | 35.878 | 11.626 | 41.118 | 1.00 | 45.48 | O |
| ATOM | 4479 | N | PRO | P | 77 | 34.092 | 10.980 | 39.889 | 1.00 | 45.19 | N |
| ATOM | 4480 | CA | PRO | P | 77 | 33.094 | 11.371 | 40.895 | 1.00 | 45.08 | C |
| ATOM | 4481 | C | PRO | P | 77 | 33.124 | 10.488 | 42.146 | 1.00 | 44.90 | C |
| ATOM | 4482 | O | PRO | P | 77 | 33.506 | 9.321 | 42.061 | 1.00 | 45.05 | O |
| ATOM | 4483 | CB | PRO | P | 77 | 31.764 | 11.181 | 40.157 | 1.00 | 45.13 | C |
| ATOM | 4484 | CG | PRO | P | 77 | 32.052 | 10.141 | 39.135 | 1.00 | 45.07 | C |
| ATOM | 4485 | CD | PRO | P | 77 | 33.466 | 10.379 | 38.697 | 1.00 | 45.11 | C |
| ATOM | 4486 | N | VAL | P | 78 | 32.726 | 11.044 | 43.288 | 1.00 | 44.69 | N |
| ATOM | 4487 | CA | VAL | P | 78 | 32.630 | 10.271 | 44.531 | 1.00 | 44.60 | C |
| ATOM | 4488 | C | VAL | P | 78 | 31.270 | 9.599 | 44.645 | 1.00 | 44.54 | C |
| ATOM | 4489 | O | VAL | P | 78 | 30.231 | 10.239 | 44.482 | 1.00 | 44.87 | O |
| ATOM | 4490 | CB | VAL | P | 78 | 32.878 | 11.134 | 45.798 | 1.00 | 44.68 | C |
| ATOM | 4491 | CG1 | VAL | P | 78 | 32.339 | 10.440 | 47.050 | 1.00 | 44.60 | C |
| ATOM | 4492 | CG2 | VAL | P | 78 | 34.360 | 11.445 | 45.964 | 1.00 | 44.35 | C |
| ATOM | 4493 | N | LEU | P | 79 | 31.286 | 8.301 | 44.919 | 1.00 | 44.26 | N |
| ATOM | 4494 | CA | LEU | P | 79 | 30.065 | 7.559 | 45.185 | 1.00 | 43.83 | C |
| ATOM | 4495 | C | LEU | P | 79 | 29.925 | 7.423 | 46.694 | 1.00 | 43.74 | C |
| ATOM | 4496 | O | LEU | P | 79 | 30.694 | 6.701 | 47.338 | 1.00 | 43.63 | O |
| ATOM | 4497 | CB | LEU | P | 79 | 30.124 | 6.189 | 44.513 | 1.00 | 43.70 | C |
| ATOM | 4498 | CG | LEU | P | 79 | 29.840 | 6.026 | 43.014 | 1.00 | 43.45 | C |
| ATOM | 4499 | CD1 | LEU | P | 79 | 30.267 | 7.205 | 42.142 | 1.00 | 43.14 | C |
| ATOM | 4500 | CD2 | LEU | P | 79 | 30.498 | 4.742 | 42.532 | 1.00 | 43.36 | C |
| ATOM | 4501 | N | LEU | P | 80 | 28.970 | 8.155 | 47.258 | 1.00 | 43.57 | N |
| ATOM | 4502 | CA | LEU | P | 80 | 28.731 | 8.136 | 48.695 | 1.00 | 43.44 | C |
| ATOM | 4503 | C | LEU | P | 80 | 27.728 | 7.035 | 48.994 | 1.00 | 43.22 | C |
| ATOM | 4504 | O | LEU | P | 80 | 26.594 | 7.084 | 48.519 | 1.00 | 43.55 | O |
| ATOM | 4505 | CB | LEU | P | 80 | 28.229 | 9.504 | 49.169 | 1.00 | 43.53 | C |
| ATOM | 4506 | CG | LEU | P | 80 | 27.981 | 9.791 | 50.653 | 1.00 | 43.75 | C |
| ATOM | 4507 | CD1 | LEU | P | 80 | 29.112 | 9.299 | 51.543 | 1.00 | 44.25 | C |
| ATOM | 4508 | CD2 | LEU | P | 80 | 27.777 | 11.280 | 50.851 | 1.00 | 43.49 | C |
| ATOM | 4509 | N | LEU | P | 81 | 28.157 | 6.041 | 49.769 | 1.00 | 42.89 | N |
| ATOM | 4510 | CA | LEU | P | 81 | 27.383 | 4.816 | 49.977 | 1.00 | 42.55 | C |
| ATOM | 4511 | C | LEU | P | 81 | 26.814 | 4.709 | 51.393 | 1.00 | 42.72 | C |
| ATOM | 4512 | O | LEU | P | 81 | 27.541 | 4.862 | 52.377 | 1.00 | 42.97 | O |
| ATOM | 4513 | CB | LEU | P | 81 | 28.245 | 3.591 | 49.659 | 1.00 | 42.39 | C |
| ATOM | 4514 | CG | LEU | P | 81 | 29.086 | 3.603 | 48.372 | 1.00 | 42.02 | C |
| ATOM | 4515 | CD1 | LEU | P | 81 | 29.934 | 2.351 | 48.255 | 1.00 | 40.77 | C |
| ATOM | 4516 | CD2 | LEU | P | 81 | 28.231 | 3.781 | 47.116 | 1.00 | 41.74 | C |
| ATOM | 4517 | N | LEU | P | 82 | 25.512 | 4.439 | 51.489 | 1.00 | 42.67 | N |
| ATOM | 4518 | CA | LEU | P | 82 | 24.819 | 4.354 | 52.778 | 1.00 | 42.22 | C |
| ATOM | 4519 | C | LEU | P | 82 | 24.126 | 3.004 | 52.961 | 1.00 | 42.08 | C |
| ATOM | 4520 | O | LEU | P | 82 | 23.156 | 2.690 | 52.267 | 1.00 | 42.24 | O |
| ATOM | 4521 | CB | LEU | P | 82 | 23.798 | 5.488 | 52.916 | 1.00 | 42.29 | C |
| ATOM | 4522 | CG | LEU | P | 82 | 24.183 | 6.920 | 52.517 | 1.00 | 42.12 | C |
| ATOM | 4523 | CD1 | LEU | P | 82 | 22.990 | 7.825 | 52.721 | 1.00 | 41.71 | C |
| ATOM | 4524 | CD2 | LEU | P | 82 | 25.389 | 7.448 | 53.296 | 1.00 | 41.63 | C |
| ATOM | 4525 | N | HIS | P | 83 | 24.631 | 2.230 | 53.918 | 1.00 | 41.70 | N |
| ATOM | 4526 | CA | HIS | P | 83 | 24.187 | 0.862 | 54.205 | 1.00 | 41.31 | C |
| ATOM | 4527 | C | HIS | P | 83 | 22.775 | 0.740 | 54.808 | 1.00 | 41.02 | C |
| ATOM | 4528 | O | HIS | P | 83 | 22.202 | 1.723 | 55.286 | 1.00 | 40.61 | O |
| ATOM | 4529 | CB | HIS | P | 83 | 25.191 | 0.220 | 55.164 | 1.00 | 41.44 | C |
| ATOM | 4530 | CG | HIS | P | 83 | 25.344 | 0.965 | 56.454 | 1.00 | 41.94 | C |
| ATOM | 4531 | ND1 | HIS | P | 83 | 24.470 | 0.814 | 57.511 | 1.00 | 42.54 | N |
| ATOM | 4532 | CD2 | HIS | P | 83 | 26.259 | 1.881 | 56.853 | 1.00 | 42.15 | C |
| ATOM | 4533 | CE1 | HIS | P | 83 | 24.843 | 1.599 | 58.505 | 1.00 | 42.74 | C |
| ATOM | 4534 | NE2 | HIS | P | 83 | 25.929 | 2.254 | 58.134 | 1.00 | 42.54 | N |
| ATOM | 4535 | N | GLY | P | 84 | 22.240 | −0.484 | 54.803 | 1.00 | 40.86 | N |
| ATOM | 4536 | CA | GLY | P | 84 | 20.967 | −0.789 | 55.457 | 1.00 | 40.73 | C |
| ATOM | 4537 | C | GLY | P | 84 | 21.056 | −0.779 | 56.980 | 1.00 | 40.79 | C |
| ATOM | 4538 | O | GLY | P | 84 | 22.138 | −0.600 | 57.552 | 1.00 | 40.48 | O |
| ATOM | 4539 | N | GLY | P | 85 | 19.911 | −0.972 | 57.637 | 1.00 | 40.80 | N |
| ATOM | 4540 | CA | GLY | P | 85 | 19.833 | −1.000 | 59.100 | 1.00 | 41.06 | C |
| ATOM | 4541 | C | GLY | P | 85 | 20.601 | −2.161 | 59.712 | 1.00 | 41.33 | C |
| ATOM | 4542 | O | GLY | P | 85 | 20.586 | −3.273 | 59.191 | 1.00 | 41.69 | O |
| ATOM | 4543 | N | GLY | P | 86 | 21.286 | −1.902 | 60.817 | 1.00 | 41.33 | N |
| ATOM | 4544 | CA | GLY | P | 86 | 22.085 | −2.921 | 61.483 | 1.00 | 41.35 | C |
| ATOM | 4545 | C | GLY | P | 86 | 23.311 | −3.382 | 60.722 | 1.00 | 41.29 | C |
| ATOM | 4546 | O | GLY | P | 86 | 23.947 | −4.360 | 61.111 | 1.00 | 41.20 | O |
| ATOM | 4547 | N | HIS | P | 87 | 23.637 | −2.690 | 59.635 | 1.00 | 41.48 | N |
| ATOM | 4548 | CA | HIS | P | 87 | 24.830 | −3.005 | 58.851 | 1.00 | 41.82 | C |
| ATOM | 4549 | C | HIS | P | 87 | 25.943 | −1.988 | 59.058 | 1.00 | 41.89 | C |
| ATOM | 4550 | O | HIS | P | 87 | 25.989 | −1.295 | 60.072 | 1.00 | 42.02 | O |
| ATOM | 4551 | CB | HIS | P | 87 | 24.496 | −3.085 | 57.370 | 1.00 | 41.86 | C |
| ATOM | 4552 | CG | HIS | P | 87 | 23.646 | −4.253 | 57.014 | 1.00 | 42.83 | C |
| ATOM | 4553 | ND1 | HIS | P | 87 | 24.163 | −5.515 | 56.825 | 1.00 | 44.16 | N |
| ATOM | 4554 | CD2 | HIS | P | 87 | 22.312 | −4.354 | 56.816 | 1.00 | 43.64 | C |
| ATOM | 4555 | CE1 | HIS | P | 87 | 23.182 | −6.346 | 56.524 | 1.00 | 45.17 | C |
| ATOM | 4556 | NE2 | HIS | P | 87 | 22.048 | −5.667 | 56.511 | 1.00 | 45.03 | N |
| ATOM | 4557 | N | SER | P | 88 | 26.834 | −1.913 | 58.076 | 1.00 | 41.96 | N |
| ATOM | 4558 | CA | SER | P | 88 | 27.996 | −1.048 | 58.122 | 1.00 | 42.01 | C |
| ATOM | 4559 | C | SER | P | 88 | 28.501 | −0.812 | 56.707 | 1.00 | 42.08 | C |
| ATOM | 4560 | O | SER | P | 88 | 28.057 | −1.465 | 55.756 | 1.00 | 42.17 | O |
| ATOM | 4561 | CB | SER | P | 88 | 29.095 | −1.700 | 58.959 | 1.00 | 42.14 | C |
| ATOM | 4562 | OG | SER | P | 88 | 29.359 | −3.015 | 58.500 | 1.00 | 42.26 | O |
| ATOM | 4563 | N | ALA | P | 89 | 29.443 | 0.117 | 56.579 | 1.00 | 42.05 | N |
| ATOM | 4564 | CA | ALA | P | 89 | 30.045 | 0.453 | 55.298 | 1.00 | 42.18 | C |
| ATOM | 4565 | C | ALA | P | 89 | 30.591 | −0.782 | 54.579 | 1.00 | 42.27 | C |
| ATOM | 4566 | O | ALA | P | 89 | 30.716 | −0.796 | 53.349 | 1.00 | 42.12 | O |
| ATOM | 4567 | CB | ALA | P | 89 | 31.148 | 1.475 | 55.502 | 1.00 | 42.02 | C |
| ATOM | 4568 | N | LEU | P | 90 | 30.900 | −1.814 | 55.362 | 1.00 | 42.44 | N |
| ATOM | 4569 | CA | LEU | P | 90 | 31.524 | −3.033 | 54.854 | 1.00 | 42.85 | C |
| ATOM | 4570 | C | LEU | P | 90 | 30.615 | −3.826 | 53.903 | 1.00 | 43.01 | C |
| ATOM | 4571 | O | LEU | P | 90 | 31.088 | −4.680 | 53.144 | 1.00 | 42.93 | O |
| ATOM | 4572 | CB | LEU | P | 90 | 32.008 | −3.910 | 56.017 | 1.00 | 42.83 | C |
| ATOM | 4573 | CG | LEU | P | 90 | 33.147 | −3.358 | 56.885 | 1.00 | 43.40 | C |
| ATOM | 4574 | CD1 | LEU | P | 90 | 33.322 | −4.166 | 58.167 | 1.00 | 43.07 | C |
| ATOM | 4575 | CD2 | LEU | P | 90 | 34.455 | −3.301 | 56.107 | 1.00 | 43.57 | C |
| ATOM | 4576 | N | SER | P | 91 | 29.317 | −3.530 | 53.940 | 1.00 | 43.11 | N |
| ATOM | 4577 | CA | SER | P | 91 | 28.366 | −4.105 | 52.998 | 1.00 | 43.36 | C |
| ATOM | 4578 | C | SER | P | 91 | 28.695 | −3.759 | 51.546 | 1.00 | 43.36 | C |
| ATOM | 4579 | O | SER | P | 91 | 28.274 | −4.459 | 50.629 | 1.00 | 43.49 | O |
| ATOM | 4580 | CB | SER | P | 91 | 26.957 | −3.640 | 53.335 | 1.00 | 43.23 | C |
| ATOM | 4581 | OG | SER | P | 91 | 26.564 | −4.167 | 54.584 | 1.00 | 44.38 | O |
| ATOM | 4582 | N | TRP | P | 92 | 29.452 | −2.684 | 51.351 | 1.00 | 43.39 | N |
| ATOM | 4583 | CA | TRP | P | 92 | 29.761 | −2.179 | 50.021 | 1.00 | 43.57 | C |
| ATOM | 4584 | C | TRP | P | 92 | 31.126 | −2.653 | 49.534 | 1.00 | 43.48 | C |
| ATOM | 4585 | O | TRP | P | 92 | 31.562 | −2.297 | 48.434 | 1.00 | 43.48 | O |
| ATOM | 4586 | CB | TRP | P | 92 | 29.711 | −0.645 | 50.017 | 1.00 | 43.82 | C |
| ATOM | 4587 | CG | TRP | P | 92 | 28.378 | −0.065 | 50.433 | 1.00 | 44.21 | C |
| ATOM | 4588 | CD1 | TRP | P | 92 | 28.027 | 0.370 | 51.682 | 1.00 | 44.48 | C |
| ATOM | 4589 | CD2 | TRP | P | 92 | 27.229 | 0.142 | 49.600 | 1.00 | 44.20 | C |
| ATOM | 4590 | NE1 | TRP | P | 92 | 26.731 | 0.831 | 51.679 | 1.00 | 44.80 | N |
| ATOM | 4591 | CE2 | TRP | P | 92 | 26.217 | 0.704 | 50.415 | 1.00 | 44.60 | C |
| ATOM | 4592 | CE3 | TRP | P | 92 | 26.954 | −0.091 | 48.246 | 1.00 | 43.75 | C |
| ATOM | 4593 | CZ2 | TRP | P | 92 | 24.950 | 1.032 | 49.921 | 1.00 | 44.16 | C |
| ATOM | 4594 | CZ3 | TRP | P | 92 | 25.694 | 0.237 | 47.756 | 1.00 | 43.96 | C |
| ATOM | 4595 | CH2 | TRP | P | 92 | 24.709 | 0.790 | 48.593 | 1.00 | 43.87 | C |
| ATOM | 4596 | N | ALA | P | 93 | 31.794 | −3.459 | 50.354 | 1.00 | 43.40 | N |
| ATOM | 4597 | CA | ALA | P | 93 | 33.175 | −3.854 | 50.093 | 1.00 | 43.33 | C |
| ATOM | 4598 | C | ALA | P | 93 | 33.360 | −4.445 | 48.699 | 1.00 | 43.36 | C |
| ATOM | 4599 | O | ALA | P | 93 | 34.183 | −3.959 | 47.915 | 1.00 | 43.24 | O |
| ATOM | 4600 | CB | ALA | P | 93 | 33.663 | −4.821 | 51.153 | 1.00 | 43.23 | C |
| ATOM | 4601 | N | VAL | P | 94 | 32.583 | −5.478 | 48.386 | 1.00 | 43.29 | N |
| ATOM | 4602 | CA | VAL | P | 94 | 32.759 | −6.186 | 47.123 | 1.00 | 43.32 | C |
| ATOM | 4603 | C | VAL | P | 94 | 32.354 | −5.304 | 45.951 | 1.00 | 43.07 | C |
| ATOM | 4604 | O | VAL | P | 94 | 33.102 | −5.177 | 44.986 | 1.00 | 43.10 | O |
| ATOM | 4605 | CB | VAL | P | 94 | 32.037 | −7.546 | 47.108 | 1.00 | 43.45 | C |
| ATOM | 4606 | CG1 | VAL | P | 94 | 32.322 | −8.290 | 45.803 | 1.00 | 44.00 | C |
| ATOM | 4607 | CG2 | VAL | P | 94 | 32.507 | −8.389 | 48.283 | 1.00 | 43.51 | C |
| ATOM | 4608 | N | PHE | P | 95 | 31.182 | −4.685 | 46.059 | 1.00 | 43.08 | N |
| ATOM | 4609 | CA | PHE | P | 95 | 30.728 | −3.671 | 45.105 | 1.00 | 42.84 | C |
| ATOM | 4610 | C | PHE | P | 95 | 31.864 | −2.726 | 44.704 | 1.00 | 42.63 | C |
| ATOM | 4611 | O | PHE | P | 95 | 32.156 | −2.560 | 43.518 | 1.00 | 42.40 | O |
| ATOM | 4612 | CB | PHE | P | 95 | 29.556 | −2.875 | 45.695 | 1.00 | 42.75 | C |
| ATOM | 4613 | CG | PHE | P | 95 | 29.174 | −1.668 | 44.887 | 1.00 | 42.88 | C |
| ATOM | 4614 | CD1 | PHE | P | 95 | 28.394 | −1.797 | 43.742 | 1.00 | 42.58 | C |
| ATOM | 4615 | CD2 | PHE | P | 95 | 29.598 | −0.400 | 45.265 | 1.00 | 43.22 | C |
| ATOM | 4616 | CE1 | PHE | P | 95 | 28.049 | −0.679 | 42.989 | 1.00 | 42.36 | C |
| ATOM | 4617 | CE2 | PHE | P | 95 | 29.252 | 0.724 | 44.512 | 1.00 | 42.92 | C |
| ATOM | 4618 | CZ | PHE | P | 95 | 28.479 | 0.581 | 43.377 | 1.00 | 42.28 | C |
| ATOM | 4619 | N | THR | P | 96 | 32.504 | −2.128 | 45.704 | 1.00 | 42.48 | N |
| ATOM | 4620 | CA | THR | P | 96 | 33.585 | −1.178 | 45.476 | 1.00 | 42.73 | C |
| ATOM | 4621 | C | THR | P | 96 | 34.660 | −1.748 | 44.539 | 1.00 | 42.77 | C |
| ATOM | 4622 | O | THR | P | 96 | 35.021 | −1.119 | 43.539 | 1.00 | 42.57 | O |
| ATOM | 4623 | CB | THR | P | 96 | 34.217 | −0.743 | 46.806 | 1.00 | 42.84 | C |
| ATOM | 4624 | OG1 | THR | P | 96 | 33.198 | −0.216 | 47.666 | 1.00 | 42.74 | O |
| ATOM | 4625 | CG2 | THR | P | 96 | 35.305 | 0.305 | 46.578 | 1.00 | 43.06 | C |
| ATOM | 4626 | N | ALA | P | 97 | 35.148 | −2.944 | 44.866 | 1.00 | 42.86 | N |
| ATOM | 4627 | CA | ALA | P | 97 | 36.142 | −3.640 | 44.050 | 1.00 | 42.86 | C |
| ATOM | 4628 | C | ALA | P | 97 | 35.678 | −3.810 | 42.594 | 1.00 | 43.01 | C |
| ATOM | 4629 | O | ALA | P | 97 | 36.460 | −3.606 | 41.653 | 1.00 | 42.81 | O |
| ATOM | 4630 | CB | ALA | P | 97 | 36.484 | −4.988 | 44.674 | 1.00 | 42.63 | C |
| ATOM | 4631 | N | ALA | P | 98 | 34.400 | −4.156 | 42.424 | 1.00 | 43.17 | N |
| ATOM | 4632 | CA | ALA | P | 98 | 33.819 | −4.397 | 41.107 | 1.00 | 43.29 | C |
| ATOM | 4633 | C | ALA | P | 98 | 33.671 | −3.111 | 40.303 | 1.00 | 43.62 | C |
| ATOM | 4634 | O | ALA | P | 98 | 33.930 | −3.100 | 39.097 | 1.00 | 43.79 | O |
| ATOM | 4635 | CB | ALA | P | 98 | 32.484 | −5.104 | 41.237 | 1.00 | 43.03 | C |
| ATOM | 4636 | N | ILE | P | 99 | 33.278 | −2.029 | 40.975 | 1.00 | 43.93 | N |
| ATOM | 4637 | CA | ILE | P | 99 | 32.978 | −0.765 | 40.298 | 1.00 | 44.09 | C |
| ATOM | 4638 | C | ILE | P | 99 | 34.238 | 0.028 | 39.918 | 1.00 | 44.56 | C |
| ATOM | 4639 | O | ILE | P | 99 | 34.303 | 0.613 | 38.835 | 1.00 | 44.61 | O |
| ATOM | 4640 | CB | ILE | P | 99 | 31.936 | 0.077 | 41.105 | 1.00 | 44.06 | C |
| ATOM | 4641 | CG1 | ILE | P | 99 | 31.045 | 0.911 | 40.166 | 1.00 | 44.10 | C |
| ATOM | 4642 | CG2 | ILE | P | 99 | 32.580 | 0.845 | 42.272 | 1.00 | 43.37 | C |
| ATOM | 4643 | CD1 | ILE | P | 99 | 31.522 | 2.301 | 39.890 | 1.00 | 44.49 | C |
| ATOM | 4644 | N | ILE | P | 100 | 35.230 | 0.027 | 40.808 | 1.00 | 44.98 | N |
| ATOM | 4645 | CA | ILE | P | 100 | 36.515 | 0.682 | 40.576 | 1.00 | 45.38 | C |
| ATOM | 4646 | C | ILE | P | 100 | 37.190 | 0.139 | 39.311 | 1.00 | 45.70 | C |
| ATOM | 4647 | O | ILE | P | 100 | 37.821 | 0.888 | 38.563 | 1.00 | 45.71 | O |
| ATOM | 4648 | CB | ILE | P | 100 | 37.433 | 0.555 | 41.841 | 1.00 | 45.55 | C |
| ATOM | 4649 | CG1 | ILE | P | 100 | 37.279 | 1.783 | 42.758 | 1.00 | 45.93 | C |
| ATOM | 4650 | CG2 | ILE | P | 100 | 38.908 | 0.264 | 41.490 | 1.00 | 45.35 | C |
| ATOM | 4651 | CD1 | ILE | P | 100 | 37.839 | 3.094 | 42.211 | 1.00 | 46.47 | C |
| ATOM | 4652 | N | SER | P | 101 | 37.029 | −1.159 | 39.067 | 1.00 | 46.13 | N |
| ATOM | 4653 | CA | SER | P | 101 | 37.591 | −1.796 | 37.879 | 1.00 | 46.48 | C |
| ATOM | 4654 | C | SER | P | 101 | 36.687 | −1.624 | 36.650 | 1.00 | 46.83 | C |
| ATOM | 4655 | O | SER | P | 101 | 36.903 | −2.274 | 35.621 | 1.00 | 47.08 | O |
| ATOM | 4656 | CB | SER | P | 101 | 37.883 | −3.278 | 38.144 | 1.00 | 46.34 | C |
| ATOM | 4657 | OG | SER | P | 101 | 36.690 | −3.985 | 38.422 | 1.00 | 46.02 | O |
| ATOM | 4658 | N | ARG | P | 102 | 35.686 | −0.750 | 36.760 | 1.00 | 47.05 | N |
| ATOM | 4659 | CA | ARG | P | 102 | 34.798 | −0.432 | 35.640 | 1.00 | 47.33 | C |
| ATOM | 4660 | C | ARG | P | 102 | 34.884 | 1.031 | 35.213 | 1.00 | 47.33 | C |
| ATOM | 4661 | O | ARG | P | 102 | 35.056 | 1.326 | 34.023 | 1.00 | 47.58 | O |
| ATOM | 4662 | CB | ARG | P | 102 | 33.351 | −0.809 | 35.956 | 1.00 | 47.43 | C |
| ATOM | 4663 | CG | ARG | P | 102 | 32.996 | −2.226 | 35.559 | 1.00 | 48.32 | C |
| ATOM | 4664 | CD | ARG | P | 102 | 31.526 | −2.534 | 35.812 | 1.00 | 49.38 | C |
| ATOM | 4665 | NE | ARG | P | 102 | 31.126 | −3.798 | 35.193 | 1.00 | 50.30 | N |
| ATOM | 4666 | CZ | ARG | P | 102 | 31.313 | −5.003 | 35.731 | 1.00 | 50.71 | C |
| ATOM | 4667 | NH1 | ARG | P | 102 | 31.906 | −5.142 | 36.915 | 1.00 | 50.05 | N |
| ATOM | 4668 | NH2 | ARG | P | 102 | 30.906 | −6.081 | 35.076 | 1.00 | 51.28 | N |
| ATOM | 4669 | N | VAL | P | 103 | 34.754 | 1.943 | 36.175 | 1.00 | 46.99 | N |
| ATOM | 4670 | CA | VAL | P | 103 | 34.874 | 3.375 | 35.894 | 1.00 | 46.60 | C |
| ATOM | 4671 | C | VAL | P | 103 | 35.838 | 4.073 | 36.846 | 1.00 | 46.41 | C |
| ATOM | 4672 | O | VAL | P | 103 | 36.076 | 3.608 | 37.960 | 1.00 | 46.44 | O |
| ATOM | 4673 | CB | VAL | P | 103 | 33.507 | 4.101 | 35.925 | 1.00 | 46.53 | C |
| ATOM | 4674 | CG1 | VAL | P | 103 | 32.690 | 3.768 | 34.691 | 1.00 | 46.47 | C |
| ATOM | 4675 | CG2 | VAL | P | 103 | 32.742 | 3.760 | 37.186 | 1.00 | 46.54 | C |
| ATOM | 4676 | N | GLN | P | 104 | 36.398 | 5.188 | 36.391 | 1.00 | 46.20 | N |
| ATOM | 4677 | CA | GLN | P | 104 | 37.237 | 6.021 | 37.242 | 1.00 | 45.90 | C |
| ATOM | 4678 | C | GLN | P | 104 | 36.332 | 6.822 | 38.173 | 1.00 | 45.48 | C |
| ATOM | 4679 | O | GLN | P | 104 | 35.753 | 7.841 | 37.787 | 1.00 | 45.51 | O |
| ATOM | 4680 | CB | GLN | P | 104 | 38.150 | 6.931 | 36.412 | 1.00 | 45.68 | C |
| ATOM | 4681 | CG | GLN | P | 104 | 39.088 | 6.180 | 35.485 | 1.00 | 45.78 | C |
| ATOM | 4682 | CD | GLN | P | 104 | 40.089 | 7.089 | 34.785 | 1.00 | 46.45 | C |
| ATOM | 4683 | OE1 | GLN | P | 104 | 41.066 | 7.553 | 35.388 | 1.00 | 46.91 | O |
| ATOM | 4684 | NE2 | GLN | P | 104 | 39.861 | 7.330 | 33.497 | 1.00 | 47.08 | N |
| ATOM | 4685 | N | CYS | P | 105 | 36.188 | 6.318 | 39.392 | 1.00 | 45.03 | N |
| ATOM | 4686 | CA | CYS | P | 105 | 35.397 | 6.978 | 40.415 | 1.00 | 44.76 | C |
| ATOM | 4687 | C | CYS | P | 105 | 36.113 | 6.910 | 41.758 | 1.00 | 44.52 | C |
| ATOM | 4688 | O | CYS | P | 105 | 37.144 | 6.246 | 41.897 | 1.00 | 44.56 | O |
| ATOM | 4689 | CB | CYS | P | 105 | 34.028 | 6.319 | 40.524 | 1.00 | 44.75 | C |
| ATOM | 4690 | SG | CYS | P | 105 | 34.129 | 4.540 | 40.777 | 1.00 | 45.28 | S |
| ATOM | 4691 | N | ARG | P | 106 | 35.575 | 7.627 | 42.736 | 1.00 | 44.10 | N |
| ATOM | 4692 | CA | ARG | P | 106 | 35.999 | 7.479 | 44.116 | 1.00 | 43.66 | C |
| ATOM | 4693 | C | ARG | P | 106 | 34.847 | 6.899 | 44.938 | 1.00 | 43.32 | C |
| ATOM | 4694 | O | ARG | P | 106 | 33.674 | 7.048 | 44.577 | 1.00 | 43.28 | O |
| ATOM | 4695 | CB | ARG | P | 106 | 36.476 | 8.814 | 44.683 | 1.00 | 43.66 | C |
| ATOM | 4696 | CG | ARG | P | 106 | 37.821 | 9.266 | 44.131 | 1.00 | 44.26 | C |
| ATOM | 4697 | CD | ARG | P | 106 | 38.306 | 10.541 | 44.802 | 1.00 | 44.98 | C |
| ATOM | 4698 | NE | ARG | P | 106 | 37.383 | 11.647 | 44.558 | 1.00 | 45.89 | N |
| ATOM | 4699 | CZ | ARG | P | 106 | 37.626 | 12.685 | 43.762 | 1.00 | 46.07 | C |
| ATOM | 4700 | NH1 | ARG | P | 106 | 38.783 | 12.797 | 43.120 | 1.00 | 46.04 | N |
| ATOM | 4701 | NH2 | ARG | P | 106 | 36.702 | 13.623 | 43.618 | 1.00 | 46.05 | N |
| ATOM | 4702 | N | ILE | P | 107 | 35.194 | 6.221 | 46.029 | 1.00 | 42.77 | N |
| ATOM | 4703 | CA | ILE | P | 107 | 34.219 | 5.575 | 46.899 | 1.00 | 42.16 | C |
| ATOM | 4704 | C | ILE | P | 107 | 34.349 | 6.111 | 48.324 | 1.00 | 42.02 | C |
| ATOM | 4705 | O | ILE | P | 107 | 35.448 | 6.162 | 48.888 | 1.00 | 41.96 | O |
| ATOM | 4706 | CB | ILE | P | 107 | 34.392 | 4.029 | 46.880 | 1.00 | 42.14 | C |
| ATOM | 4707 | CG1 | ILE | P | 107 | 34.013 | 3.455 | 45.505 | 1.00 | 41.89 | C |
| ATOM | 4708 | CG2 | ILE | P | 107 | 33.632 | 3.348 | 48.043 | 1.00 | 41.55 | C |
| ATOM | 4709 | CD1 | ILE | P | 107 | 32.517 | 3.297 | 45.251 | 1.00 | 41.70 | C |
| ATOM | 4710 | N | VAL | P | 108 | 33.220 | 6.536 | 48.883 | 1.00 | 41.64 | N |
| ATOM | 4711 | CA | VAL | P | 108 | 33.138 | 6.887 | 50.296 | 1.00 | 41.27 | C |
| ATOM | 4712 | C | VAL | P | 108 | 31.989 | 6.102 | 50.920 | 1.00 | 41.04 | C |
| ATOM | 4713 | O | VAL | P | 108 | 30.820 | 6.475 | 50.799 | 1.00 | 41.13 | O |
| ATOM | 4714 | CB | VAL | P | 108 | 32.969 | 8.411 | 50.515 | 1.00 | 41.36 | C |
| ATOM | 4715 | CG1 | VAL | P | 108 | 32.651 | 8.717 | 51.979 | 1.00 | 41.12 | C |
| ATOM | 4716 | CG2 | VAL | P | 108 | 34.231 | 9.161 | 50.068 | 1.00 | 41.02 | C |
| ATOM | 4717 | N | ALA | P | 109 | 32.339 | 4.991 | 51.559 | 1.00 | 40.58 | N |
| ATOM | 4718 | CA | ALA | P | 109 | 31.368 | 4.121 | 52.199 | 1.00 | 39.98 | C |
| ATOM | 4719 | C | ALA | P | 109 | 31.283 | 4.498 | 53.673 | 1.00 | 39.71 | C |
| ATOM | 4720 | O | ALA | P | 109 | 32.222 | 4.262 | 54.432 | 1.00 | 39.83 | O |
| ATOM | 4721 | CB | ALA | P | 109 | 31.786 | 2.670 | 52.028 | 1.00 | 39.92 | C |
| ATOM | 4722 | N | LEU | P | 110 | 30.161 | 5.088 | 54.073 | 1.00 | 39.33 | N |
| ATOM | 4723 | CA | LEU | P | 110 | 30.027 | 5.665 | 55.412 | 1.00 | 38.93 | C |
| ATOM | 4724 | C | LEU | P | 110 | 29.337 | 4.752 | 56.418 | 1.00 | 38.86 | C |
| ATOM | 4725 | O | LEU | P | 110 | 28.254 | 4.248 | 56.156 | 1.00 | 38.73 | O |
| ATOM | 4726 | CB | LEU | P | 110 | 29.290 | 7.006 | 55.323 | 1.00 | 38.89 | C |
| ATOM | 4727 | CG | LEU | P | 110 | 28.670 | 7.633 | 56.573 | 1.00 | 38.75 | C |
| ATOM | 4728 | CD1 | LEU | P | 110 | 29.737 | 8.074 | 57.566 | 1.00 | 39.62 | C |
| ATOM | 4729 | CD2 | LEU | P | 110 | 27.798 | 8.815 | 56.182 | 1.00 | 38.55 | C |
| ATOM | 4730 | N | ASP | P | 111 | 29.981 | 4.537 | 57.565 | 1.00 | 39.01 | N |
| ATOM | 4731 | CA | ASP | P | 111 | 29.331 | 3.912 | 58.724 | 1.00 | 39.15 | C |
| ATOM | 4732 | C | ASP | P | 111 | 28.383 | 4.946 | 59.335 | 1.00 | 39.16 | C |
| ATOM | 4733 | O | ASP | P | 111 | 28.838 | 5.943 | 59.883 | 1.00 | 39.49 | O |
| ATOM | 4734 | CB | ASP | P | 111 | 30.362 | 3.499 | 59.788 | 1.00 | 39.04 | C |
| ATOM | 4735 | CG | ASP | P | 111 | 30.926 | 2.094 | 59.581 | 1.00 | 39.19 | C |
| ATOM | 4736 | OD1 | ASP | P | 111 | 30.388 | 1.332 | 58.748 | 1.00 | 39.49 | O |
| ATOM | 4737 | OD2 | ASP | P | 111 | 31.914 | 1.750 | 60.281 | 1.00 | 38.32 | O |
| ATOM | 4738 | N | LEU | P | 112 | 27.078 | 4.719 | 59.235 | 1.00 | 39.05 | N |
| ATOM | 4739 | CA | LEU | P | 112 | 26.093 | 5.633 | 59.814 | 1.00 | 39.11 | C |
| ATOM | 4740 | C | LEU | P | 112 | 26.094 | 5.560 | 61.346 | 1.00 | 38.98 | C |
| ATOM | 4741 | O | LEU | P | 112 | 26.674 | 4.640 | 61.920 | 1.00 | 38.80 | O |
| ATOM | 4742 | CB | LEU | P | 112 | 24.696 | 5.350 | 59.244 | 1.00 | 39.29 | C |
| ATOM | 4743 | CG | LEU | P | 112 | 24.449 | 5.786 | 57.792 | 1.00 | 39.68 | C |
| ATOM | 4744 | CD1 | LEU | P | 112 | 23.441 | 4.880 | 57.085 | 1.00 | 39.26 | C |
| ATOM | 4745 | CD2 | LEU | P | 112 | 24.021 | 7.252 | 57.714 | 1.00 | 39.91 | C |
| ATOM | 4746 | N | ARG | P | 113 | 25.453 | 6.531 | 62.002 | 1.00 | 39.03 | N |
| ATOM | 4747 | CA | ARG | P | 113 | 25.470 | 6.618 | 63.470 | 1.00 | 39.17 | C |
| ATOM | 4748 | C | ARG | P | 113 | 24.934 | 5.353 | 64.125 | 1.00 | 39.25 | C |
| ATOM | 4749 | O | ARG | P | 113 | 24.036 | 4.697 | 63.589 | 1.00 | 39.25 | O |
| ATOM | 4750 | CB | ARG | P | 113 | 24.739 | 7.870 | 63.984 | 1.00 | 39.11 | C |
| ATOM | 4751 | CG | ARG | P | 113 | 23.236 | 7.734 | 64.185 | 1.00 | 39.19 | C |
| ATOM | 4752 | CD | ARG | P | 113 | 22.539 | 9.092 | 64.123 | 1.00 | 39.70 | C |
| ATOM | 4753 | NE | ARG | P | 113 | 22.537 | 9.805 | 65.399 | 1.00 | 39.83 | N |
| ATOM | 4754 | CZ | ARG | P | 113 | 23.375 | 10.788 | 65.733 | 1.00 | 40.08 | C |
| ATOM | 4755 | NH1 | ARG | P | 113 | 24.317 | 11.204 | 64.898 | 1.00 | 39.92 | N |
| ATOM | 4756 | NH2 | ARG | P | 113 | 23.272 | 11.361 | 66.924 | 1.00 | 40.05 | N |
| ATOM | 4757 | N | SER | P | 114 | 25.525 | 5.008 | 65.268 | 1.00 | 39.28 | N |
| ATOM | 4758 | CA | SER | P | 114 | 25.227 | 3.771 | 65.998 | 1.00 | 39.29 | C |
| ATOM | 4759 | C | SER | P | 114 | 25.657 | 2.499 | 65.255 | 1.00 | 39.27 | C |
| ATOM | 4760 | O | SER | P | 114 | 25.573 | 1.399 | 65.806 | 1.00 | 39.26 | O |
| ATOM | 4761 | CB | SER | P | 114 | 23.747 | 3.708 | 66.376 | 1.00 | 39.25 | C |
| ATOM | 4762 | OG | SER | P | 114 | 23.339 | 4.915 | 66.991 | 1.00 | 39.49 | O |
| ATOM | 4763 | N | HIS | P | 115 | 26.130 | 2.656 | 64.018 | 1.00 | 39.24 | N |
| ATOM | 4764 | CA | HIS | P | 115 | 26.560 | 1.526 | 63.193 | 1.00 | 39.27 | C |
| ATOM | 4765 | C | HIS | P | 115 | 28.076 | 1.473 | 63.025 | 1.00 | 39.45 | C |
| ATOM | 4766 | O | HIS | P | 115 | 28.765 | 2.483 | 63.189 | 1.00 | 39.36 | O |
| ATOM | 4767 | CB | HIS | P | 115 | 25.894 | 1.586 | 61.818 | 1.00 | 39.26 | C |
| ATOM | 4768 | CG | HIS | P | 115 | 24.412 | 1.381 | 61.855 | 1.00 | 38.95 | C |
| ATOM | 4769 | ND1 | HIS | P | 115 | 23.536 | 2.361 | 62.270 | 1.00 | 38.67 | N |
| ATOM | 4770 | CD2 | HIS | P | 115 | 23.652 | 0.310 | 61.530 | 1.00 | 38.41 | C |
| ATOM | 4771 | CE1 | HIS | P | 115 | 22.301 | 1.901 | 62.201 | 1.00 | 38.24 | C |
| ATOM | 4772 | NE2 | HIS | P | 115 | 22.344 | 0.660 | 61.753 | 1.00 | 38.27 | N |
| ATOM | 4773 | N | GLY | P | 116 | 28.581 | 0.287 | 62.696 | 1.00 | 39.70 | N |
| ATOM | 4774 | CA | GLY | P | 116 | 30.006 | 0.076 | 62.456 | 1.00 | 40.18 | C |
| ATOM | 4775 | C | GLY | P | 116 | 30.862 | 0.609 | 63.584 | 1.00 | 40.46 | C |
| ATOM | 4776 | O | GLY | P | 116 | 30.654 | 0.243 | 64.743 | 1.00 | 40.57 | O |
| ATOM | 4777 | N | GLU | P | 117 | 31.809 | 1.487 | 63.247 | 1.00 | 40.74 | N |
| ATOM | 4778 | CA | GLU | P | 117 | 32.707 | 2.083 | 64.248 | 1.00 | 41.03 | C |
| ATOM | 4779 | C | GLU | P | 117 | 32.441 | 3.574 | 64.486 | 1.00 | 41.34 | C |
| ATOM | 4780 | O | GLU | P | 117 | 33.342 | 4.312 | 64.880 | 1.00 | 41.48 | O |
| ATOM | 4781 | CB | GLU | P | 117 | 34.186 | 1.845 | 63.889 | 1.00 | 40.80 | C |
| ATOM | 4782 | CG | GLU | P | 117 | 34.629 | 0.375 | 63.872 | 1.00 | 40.27 | C |
| ATOM | 4783 | CD | GLU | P | 117 | 34.763 | −0.246 | 65.254 | 1.00 | 39.72 | C |
| ATOM | 4784 | OE1 | GLU | P | 117 | 34.924 | 0.489 | 66.251 | 1.00 | 39.47 | O |
| ATOM | 4785 | OE2 | GLU | P | 117 | 34.718 | −1.487 | 65.343 | 1.00 | 39.76 | O |
| ATOM | 4786 | N | THR | P | 118 | 31.200 | 4.001 | 64.267 | 1.00 | 41.77 | N |
| ATOM | 4787 | CA | THR | P | 118 | 30.810 | 5.398 | 64.437 | 1.00 | 42.24 | C |
| ATOM | 4788 | C | THR | P | 118 | 30.432 | 5.710 | 65.882 | 1.00 | 42.64 | C |
| ATOM | 4789 | O | THR | P | 118 | 29.381 | 5.296 | 66.374 | 1.00 | 42.46 | O |
| ATOM | 4790 | CB | THR | P | 118 | 29.650 | 5.769 | 63.496 | 1.00 | 42.24 | C |
| ATOM | 4791 | OG1 | THR | P | 118 | 30.053 | 5.541 | 62.147 | 1.00 | 42.09 | O |
| ATOM | 4792 | CG2 | THR | P | 118 | 29.268 | 7.225 | 63.643 | 1.00 | 42.61 | C |
| ATOM | 4793 | N | LYS | P | 119 | 31.304 | 6.455 | 66.548 | 1.00 | 43.52 | N |
| ATOM | 4794 | CA | LYS | P | 119 | 31.120 | 6.812 | 67.950 | 1.00 | 44.34 | C |
| ATOM | 4795 | C | LYS | P | 119 | 30.456 | 8.182 | 68.018 | 1.00 | 44.75 | C |
| ATOM | 4796 | O | LYS | P | 119 | 31.037 | 9.188 | 67.611 | 1.00 | 44.80 | O |
| ATOM | 4797 | CB | LYS | P | 119 | 32.460 | 6.837 | 68.697 | 1.00 | 44.25 | C |
| ATOM | 4798 | CG | LYS | P | 119 | 33.625 | 6.135 | 67.995 | 1.00 | 44.88 | C |
| ATOM | 4799 | CD | LYS | P | 119 | 33.653 | 4.624 | 68.228 | 1.00 | 45.90 | C |
| ATOM | 4800 | CE | LYS | P | 119 | 34.972 | 4.035 | 67.723 | 1.00 | 46.49 | C |
| ATOM | 4801 | NZ | LYS | P | 119 | 35.139 | 2.595 | 68.048 | 1.00 | 46.64 | N |
| ATOM | 4802 | N | VAL | P | 120 | 29.227 | 8.206 | 68.520 | 1.00 | 45.33 | N |
| ATOM | 4803 | CA | VAL | P | 120 | 28.444 | 9.429 | 68.592 | 1.00 | 45.89 | C |
| ATOM | 4804 | C | VAL | P | 120 | 28.040 | 9.694 | 70.041 | 1.00 | 46.38 | C |
| ATOM | 4805 | O | VAL | P | 120 | 27.670 | 8.765 | 70.764 | 1.00 | 46.63 | O |
| ATOM | 4806 | CB | VAL | P | 120 | 27.190 | 9.333 | 67.698 | 1.00 | 45.86 | C |
| ATOM | 4807 | CG1 | VAL | P | 120 | 26.351 | 10.591 | 67.805 | 1.00 | 46.29 | C |
| ATOM | 4808 | CG2 | VAL | P | 120 | 27.587 | 9.098 | 66.251 | 1.00 | 45.95 | C |
| ATOM | 4809 | N | LYS | P | 121 | 28.124 | 10.958 | 70.460 | 1.00 | 46.84 | N |
| ATOM | 4810 | CA | LYS | P | 121 | 27.711 | 11.374 | 71.808 | 1.00 | 47.12 | C |
| ATOM | 4811 | C | LYS | P | 121 | 26.264 | 10.948 | 72.099 | 1.00 | 47.38 | C |
| ATOM | 4812 | O | LYS | P | 121 | 25.923 | 10.613 | 73.239 | 1.00 | 47.54 | O |
| ATOM | 4813 | CB | LYS | P | 121 | 27.901 | 12.894 | 71.984 | 1.00 | 46.93 | C |
| ATOM | 4814 | CG | LYS | P | 121 | 27.158 | 13.547 | 73.164 | 1.00 | 47.01 | C |
| ATOM | 4815 | CD | LYS | P | 121 | 27.756 | 13.202 | 74.531 | 1.00 | 46.64 | C |
| ATOM | 4816 | CE | LYS | P | 121 | 26.966 | 13.854 | 75.659 | 1.00 | 46.16 | C |
| ATOM | 4817 | NZ | LYS | P | 121 | 27.477 | 13.444 | 76.991 | 1.00 | 45.57 | N |
| ATOM | 4818 | N | ASN | P | 122 | 25.436 | 10.943 | 71.054 | 1.00 | 47.48 | N |
| ATOM | 4819 | CA | ASN | P | 122 | 24.032 | 10.561 | 71.161 | 1.00 | 47.68 | C |
| ATOM | 4820 | C | ASN | P | 122 | 23.658 | 9.444 | 70.166 | 1.00 | 47.83 | C |
| ATOM | 4821 | O | ASN | P | 122 | 23.196 | 9.724 | 69.053 | 1.00 | 47.79 | O |
| ATOM | 4822 | CB | ASN | P | 122 | 23.150 | 11.801 | 70.975 | 1.00 | 47.75 | C |
| ATOM | 4823 | CG | ASN | P | 122 | 21.676 | 11.522 | 71.210 | 1.00 | 47.99 | C |
| ATOM | 4824 | OD1 | ASN | P | 122 | 21.307 | 10.550 | 71.870 | 1.00 | 48.43 | O |
| ATOM | 4825 | ND2 | ASN | P | 122 | 20.823 | 12.383 | 70.666 | 1.00 | 48.21 | N |
| ATOM | 4826 | N | PRO | P | 123 | 23.860 | 8.170 | 70.565 | 1.00 | 47.89 | N |
| ATOM | 4827 | CA | PRO | P | 123 | 23.622 | 7.061 | 69.649 | 1.00 | 47.95 | C |
| ATOM | 4828 | C | PRO | P | 123 | 22.190 | 6.520 | 69.704 | 1.00 | 48.07 | C |
| ATOM | 4829 | O | PRO | P | 123 | 21.886 | 5.522 | 69.048 | 1.00 | 48.05 | O |
| ATOM | 4830 | CB | PRO | P | 123 | 24.620 | 6.006 | 70.133 | 1.00 | 47.92 | C |
| ATOM | 4831 | CG | PRO | P | 123 | 24.769 | 6.268 | 71.600 | 1.00 | 47.84 | C |
| ATOM | 4832 | CD | PRO | P | 123 | 24.326 | 7.691 | 71.879 | 1.00 | 47.92 | C |
| ATOM | 4833 | N | GLU | P | 124 | 21.323 | 7.176 | 70.471 | 1.00 | 48.19 | N |
| ATOM | 4834 | CA | GLU | P | 124 | 19.936 | 6.734 | 70.612 | 1.00 | 48.48 | C |
| ATOM | 4835 | C | GLU | P | 124 | 18.991 | 7.386 | 69.580 | 1.00 | 48.56 | C |
| ATOM | 4836 | O | GLU | P | 124 | 17.973 | 6.797 | 69.199 | 1.00 | 48.61 | O |
| ATOM | 4837 | CB | GLU | P | 124 | 19.455 | 6.962 | 72.050 | 1.00 | 48.53 | C |
| ATOM | 4838 | CG | GLU | P | 124 | 18.273 | 6.096 | 72.473 | 1.00 | 49.03 | C |
| ATOM | 4839 | CD | GLU | P | 124 | 16.972 | 6.879 | 72.535 | 1.00 | 49.93 | C |
| ATOM | 4840 | OE1 | GLU | P | 124 | 16.750 | 7.575 | 73.552 | 1.00 | 49.80 | O |
| ATOM | 4841 | OE2 | GLU | P | 124 | 16.170 | 6.792 | 71.576 | 1.00 | 50.18 | O |
| ATOM | 4842 | N | ASP | P | 125 | 19.350 | 8.582 | 69.116 | 1.00 | 48.55 | N |
| ATOM | 4843 | CA | ASP | P | 125 | 18.543 | 9.331 | 68.147 | 1.00 | 48.48 | C |
| ATOM | 4844 | C | ASP | P | 125 | 18.817 | 8.915 | 66.696 | 1.00 | 48.34 | C |
| ATOM | 4845 | O | ASP | P | 125 | 19.804 | 9.347 | 66.090 | 1.00 | 48.34 | O |
| ATOM | 4846 | CB | ASP | P | 125 | 18.773 | 10.837 | 68.333 | 1.00 | 48.60 | C |
| ATOM | 4847 | CG | ASP | P | 125 | 18.039 | 11.682 | 67.306 | 1.00 | 48.98 | C |
| ATOM | 4848 | OD1 | ASP | P | 125 | 16.958 | 11.273 | 66.828 | 1.00 | 49.56 | O |
| ATOM | 4849 | OD2 | ASP | P | 125 | 18.547 | 12.773 | 66.981 | 1.00 | 49.47 | O |
| ATOM | 4850 | N | LEU | P | 126 | 17.930 | 8.087 | 66.143 | 1.00 | 48.11 | N |
| ATOM | 4851 | CA | LEU | P | 126 | 18.080 | 7.585 | 64.774 | 1.00 | 47.90 | C |
| ATOM | 4852 | C | LEU | P | 126 | 16.922 | 8.001 | 63.872 | 1.00 | 47.86 | C |
| ATOM | 4853 | O | LEU | P | 126 | 16.380 | 7.188 | 63.117 | 1.00 | 47.74 | O |
| ATOM | 4854 | CB | LEU | P | 126 | 18.253 | 6.067 | 64.771 | 1.00 | 47.79 | C |
| ATOM | 4855 | CG | LEU | P | 126 | 19.644 | 5.590 | 65.181 | 1.00 | 47.85 | C |
| ATOM | 4856 | CD1 | LEU | P | 126 | 19.547 | 4.505 | 66.235 | 1.00 | 48.35 | C |
| ATOM | 4857 | CD2 | LEU | P | 126 | 20.438 | 5.119 | 63.977 | 1.00 | 47.85 | C |
| ATOM | 4858 | N | SER | P | 127 | 16.556 | 9.276 | 63.958 | 1.00 | 47.70 | N |
| ATOM | 4859 | CA | SER | P | 127 | 15.526 | 9.842 | 63.106 | 1.00 | 47.63 | C |
| ATOM | 4860 | C | SER | P | 127 | 16.085 | 10.052 | 61.706 | 1.00 | 47.55 | C |
| ATOM | 4861 | O | SER | P | 127 | 17.304 | 10.168 | 61.528 | 1.00 | 47.60 | O |
| ATOM | 4862 | CB | SER | P | 127 | 15.022 | 11.166 | 63.685 | 1.00 | 47.73 | C |
| ATOM | 4863 | OG | SER | P | 127 | 16.073 | 12.109 | 63.795 | 1.00 | 47.93 | O |
| ATOM | 4864 | N | ALA | P | 128 | 15.188 | 10.091 | 60.721 | 1.00 | 47.33 | N |
| ATOM | 4865 | CA | ALA | P | 128 | 15.562 | 10.282 | 59.323 | 1.00 | 47.09 | C |
| ATOM | 4866 | C | ALA | P | 128 | 16.366 | 11.564 | 59.141 | 1.00 | 47.08 | C |
| ATOM | 4867 | O | ALA | P | 128 | 17.348 | 11.594 | 58.396 | 1.00 | 46.98 | O |
| ATOM | 4868 | CB | ALA | P | 128 | 14.320 | 10.302 | 58.441 | 1.00 | 46.99 | C |
| ATOM | 4869 | N | GLU | P | 129 | 15.950 | 12.611 | 59.849 | 1.00 | 47.05 | N |
| ATOM | 4870 | CA | GLU | P | 129 | 16.556 | 13.934 | 59.729 | 1.00 | 47.09 | C |
| ATOM | 4871 | C | GLU | P | 129 | 17.961 | 13.955 | 60.314 | 1.00 | 46.60 | C |
| ATOM | 4872 | O | GLU | P | 129 | 18.890 | 14.451 | 59.677 | 1.00 | 46.91 | O |
| ATOM | 4873 | CB | GLU | P | 129 | 15.691 | 15.011 | 60.401 | 1.00 | 47.30 | C |
| ATOM | 4874 | CG | GLU | P | 129 | 14.188 | 14.826 | 60.214 | 1.00 | 48.47 | C |
| ATOM | 4875 | CD | GLU | P | 129 | 13.608 | 13.810 | 61.185 | 1.00 | 49.69 | C |
| ATOM | 4876 | OE1 | GLU | P | 129 | 13.282 | 12.686 | 60.738 | 1.00 | 49.46 | O |
| ATOM | 4877 | OE2 | GLU | P | 129 | 13.506 | 14.131 | 62.394 | 1.00 | 50.24 | O |
| ATOM | 4878 | N | THR | P | 130 | 18.114 | 13.411 | 61.518 | 1.00 | 45.81 | N |
| ATOM | 4879 | CA | THR | P | 130 | 19.408 | 13.422 | 62.191 | 1.00 | 45.03 | C |
| ATOM | 4880 | C | THR | P | 130 | 20.461 | 12.661 | 61.382 | 1.00 | 44.76 | C |
| ATOM | 4881 | O | THR | P | 130 | 21.597 | 13.118 | 61.254 | 1.00 | 44.75 | O |
| ATOM | 4882 | CB | THR | P | 130 | 19.316 | 12.862 | 63.625 | 1.00 | 44.90 | C |
| ATOM | 4883 | OG1 | THR | P | 130 | 18.284 | 13.552 | 64.339 | 1.00 | 44.05 | O |
| ATOM | 4884 | CG2 | THR | P | 130 | 20.636 | 13.043 | 64.360 | 1.00 | 44.78 | C |
| ATOM | 4885 | N | MET | P | 131 | 20.070 | 11.520 | 60.822 | 1.00 | 44.31 | N |
| ATOM | 4886 | CA | MET | P | 131 | 21.000 | 10.661 | 60.096 | 1.00 | 43.89 | C |
| ATOM | 4887 | C | MET | P | 131 | 21.397 | 11.272 | 58.758 | 1.00 | 43.71 | C |
| ATOM | 4888 | O | MET | P | 131 | 22.511 | 11.055 | 58.283 | 1.00 | 43.66 | O |
| ATOM | 4889 | CB | MET | P | 131 | 20.406 | 9.265 | 59.895 | 1.00 | 43.90 | C |
| ATOM | 4890 | CG | MET | P | 131 | 20.241 | 8.468 | 61.181 | 1.00 | 43.68 | C |
| ATOM | 4891 | SD | MET | P | 131 | 19.645 | 6.783 | 60.915 | 1.00 | 43.92 | S |
| ATOM | 4892 | CE | MET | P | 131 | 21.114 | 5.975 | 60.280 | 1.00 | 43.28 | C |
| ATOM | 4893 | N | ALA | P | 132 | 20.476 | 12.030 | 58.161 | 1.00 | 43.53 | N |
| ATOM | 4894 | CA | ALA | P | 132 | 20.720 | 12.745 | 56.906 | 1.00 | 43.30 | C |
| ATOM | 4895 | C | ALA | P | 132 | 21.607 | 13.964 | 57.157 | 1.00 | 43.26 | C |
| ATOM | 4896 | O | ALA | P | 132 | 22.516 | 14.260 | 56.369 | 1.00 | 43.28 | O |
| ATOM | 4897 | CB | ALA | P | 132 | 19.404 | 13.165 | 56.275 | 1.00 | 43.21 | C |
| ATOM | 4898 | N | LYS | P | 133 | 21.325 | 14.658 | 58.261 | 1.00 | 43.01 | N |
| ATOM | 4899 | CA | LYS | P | 133 | 22.144 | 15.756 | 58.754 | 1.00 | 42.80 | C |
| ATOM | 4900 | C | LYS | P | 133 | 23.586 | 15.278 | 58.917 | 1.00 | 42.55 | C |
| ATOM | 4901 | O | LYS | P | 133 | 24.529 | 16.006 | 58.601 | 1.00 | 42.67 | O |
| ATOM | 4902 | CB | LYS | P | 133 | 21.591 | 16.255 | 60.090 | 1.00 | 42.74 | C |
| ATOM | 4903 | CG | LYS | P | 133 | 22.311 | 17.461 | 60.682 | 1.00 | 43.64 | C |
| ATOM | 4904 | CD | LYS | P | 133 | 21.509 | 18.756 | 60.549 | 1.00 | 45.11 | C |
| ATOM | 4905 | CE | LYS | P | 133 | 21.734 | 19.458 | 59.211 | 1.00 | 45.94 | C |
| ATOM | 4906 | NZ | LYS | P | 133 | 21.245 | 20.870 | 59.246 | 1.00 | 46.13 | N |
| ATOM | 4907 | N | ASP | P | 134 | 23.742 | 14.044 | 59.392 | 1.00 | 42.19 | N |
| ATOM | 4908 | CA | ASP | P | 134 | 25.057 | 13.452 | 59.610 | 1.00 | 41.89 | C |
| ATOM | 4909 | C | ASP | P | 134 | 25.806 | 13.273 | 58.309 | 1.00 | 41.77 | C |
| ATOM | 4910 | O | ASP | P | 134 | 26.978 | 13.647 | 58.214 | 1.00 | 41.90 | O |
| ATOM | 4911 | CB | ASP | P | 134 | 24.939 | 12.108 | 60.321 | 1.00 | 41.70 | C |
| ATOM | 4912 | CG | ASP | P | 134 | 24.549 | 12.249 | 61.775 | 1.00 | 41.66 | C |
| ATOM | 4913 | OD1 | ASP | P | 134 | 24.470 | 13.389 | 62.288 | 1.00 | 40.97 | O |
| ATOM | 4914 | OD2 | ASP | P | 134 | 24.318 | 11.202 | 62.407 | 1.00 | 41.92 | O |
| ATOM | 4915 | N | VAL | P | 135 | 25.121 | 12.706 | 57.317 | 1.00 | 41.43 | N |
| ATOM | 4916 | CA | VAL | P | 135 | 25.687 | 12.504 | 55.987 | 1.00 | 41.12 | C |
| ATOM | 4917 | C | VAL | P | 135 | 26.225 | 13.820 | 55.435 | 1.00 | 41.14 | C |
| ATOM | 4918 | O | VAL | P | 135 | 27.362 | 13.883 | 54.966 | 1.00 | 41.09 | O |
| ATOM | 4919 | CB | VAL | P | 135 | 24.649 | 11.920 | 55.003 | 1.00 | 41.08 | C |
| ATOM | 4920 | CG1 | VAL | P | 135 | 25.307 | 11.576 | 53.672 | 1.00 | 40.84 | C |
| ATOM | 4921 | CG2 | VAL | P | 135 | 23.989 | 10.695 | 55.593 | 1.00 | 40.77 | C |
| ATOM | 4922 | N | GLY | P | 136 | 25.405 | 14.868 | 55.511 | 1.00 | 41.14 | N |
| ATOM | 4923 | CA | GLY | P | 136 | 25.788 | 16.192 | 55.033 | 1.00 | 41.15 | C |
| ATOM | 4924 | C | GLY | P | 136 | 27.036 | 16.703 | 55.721 | 1.00 | 41.29 | C |
| ATOM | 4925 | O | GLY | P | 136 | 27.927 | 17.260 | 55.078 | 1.00 | 41.39 | O |
| ATOM | 4926 | N | ASN | P | 137 | 27.100 | 16.494 | 57.034 | 1.00 | 41.34 | N |
| ATOM | 4927 | CA | ASN | P | 137 | 28.208 | 16.978 | 57.849 | 1.00 | 41.34 | C |
| ATOM | 4928 | C | ASN | P | 137 | 29.524 | 16.265 | 57.579 | 1.00 | 41.33 | C |
| ATOM | 4929 | O | ASN | P | 137 | 30.592 | 16.876 | 57.655 | 1.00 | 41.26 | O |
| ATOM | 4930 | CB | ASN | P | 137 | 27.848 | 16.893 | 59.332 | 1.00 | 41.39 | C |
| ATOM | 4931 | CG | ASN | P | 137 | 26.854 | 17.958 | 59.751 | 1.00 | 41.42 | C |
| ATOM | 4932 | OD1 | ASN | P | 137 | 26.525 | 18.861 | 58.980 | 1.00 | 41.63 | O |
| ATOM | 4933 | ND2 | ASN | P | 137 | 26.368 | 17.859 | 60.979 | 1.00 | 41.74 | N |
| ATOM | 4934 | N | VAL | P | 138 | 29.436 | 14.975 | 57.264 | 1.00 | 41.39 | N |
| ATOM | 4935 | CA | VAL | P | 138 | 30.607 | 14.182 | 56.898 | 1.00 | 41.39 | C |
| ATOM | 4936 | C | VAL | P | 138 | 31.204 | 14.751 | 55.614 | 1.00 | 41.56 | C |
| ATOM | 4937 | O | VAL | P | 138 | 32.423 | 14.877 | 55.495 | 1.00 | 41.57 | O |
| ATOM | 4938 | CB | VAL | P | 138 | 30.259 | 12.680 | 56.729 | 1.00 | 41.26 | C |
| ATOM | 4939 | CG1 | VAL | P | 138 | 31.455 | 11.896 | 56.201 | 1.00 | 41.14 | C |
| ATOM | 4940 | CG2 | VAL | P | 138 | 29.788 | 12.087 | 58.050 | 1.00 | 41.17 | C |
| ATOM | 4941 | N | VAL | P | 139 | 30.332 | 15.113 | 54.675 | 1.00 | 41.74 | N |
| ATOM | 4942 | CA | VAL | P | 139 | 30.739 | 15.757 | 53.427 | 1.00 | 42.03 | C |
| ATOM | 4943 | C | VAL | P | 139 | 31.490 | 17.063 | 53.700 | 1.00 | 42.25 | C |
| ATOM | 4944 | O | VAL | P | 139 | 32.616 | 17.241 | 53.230 | 1.00 | 42.33 | O |
| ATOM | 4945 | CB | VAL | P | 139 | 29.524 | 16.006 | 52.492 | 1.00 | 42.23 | C |
| ATOM | 4946 | CG1 | VAL | P | 139 | 29.920 | 16.855 | 51.271 | 1.00 | 41.83 | C |
| ATOM | 4947 | CG2 | VAL | P | 139 | 28.889 | 14.673 | 52.065 | 1.00 | 41.86 | C |
| ATOM | 4948 | N | GLU | P | 140 | 30.878 | 17.963 | 54.471 | 1.00 | 42.41 | N |
| ATOM | 4949 | CA | GLU | P | 140 | 31.527 | 19.227 | 54.816 | 1.00 | 42.73 | C |
| ATOM | 4950 | C | GLU | P | 140 | 32.862 | 19.004 | 55.533 | 1.00 | 42.74 | C |
| ATOM | 4951 | O | GLU | P | 140 | 33.870 | 19.604 | 55.167 | 1.00 | 42.60 | O |
| ATOM | 4952 | CB | GLU | P | 140 | 30.608 | 20.123 | 55.654 | 1.00 | 42.80 | C |
| ATOM | 4953 | CG | GLU | P | 140 | 31.127 | 21.558 | 55.817 | 1.00 | 42.75 | C |
| ATOM | 4954 | CD | GLU | P | 140 | 30.431 | 22.332 | 56.930 | 1.00 | 43.03 | C |
| ATOM | 4955 | OE1 | GLU | P | 140 | 31.005 | 23.341 | 57.413 | 1.00 | 44.02 | O |
| ATOM | 4956 | OE2 | GLU | P | 140 | 29.312 | 21.940 | 57.325 | 1.00 | 43.09 | O |
| ATOM | 4957 | N | ALA | P | 141 | 32.863 | 18.128 | 56.535 | 1.00 | 42.88 | N |
| ATOM | 4958 | CA | ALA | P | 141 | 34.069 | 17.839 | 57.313 | 1.00 | 43.20 | C |
| ATOM | 4959 | C | ALA | P | 141 | 35.208 | 17.253 | 56.473 | 1.00 | 43.41 | C |
| ATOM | 4960 | O | ALA | P | 141 | 36.377 | 17.567 | 56.705 | 1.00 | 43.31 | O |
| ATOM | 4961 | CB | ALA | P | 141 | 33.742 | 16.925 | 58.486 | 1.00 | 43.19 | C |
| ATOM | 4962 | N | MET | P | 142 | 34.862 | 16.412 | 55.500 | 1.00 | 43.78 | N |
| ATOM | 4963 | CA | MET | P | 142 | 35.853 | 15.812 | 54.606 | 1.00 | 44.11 | C |
| ATOM | 4964 | C | MET | P | 142 | 36.361 | 16.797 | 53.558 | 1.00 | 44.38 | C |
| ATOM | 4965 | O | MET | P | 142 | 37.557 | 16.810 | 53.258 | 1.00 | 44.47 | O |
| ATOM | 4966 | CB | MET | P | 142 | 35.293 | 14.564 | 53.924 | 1.00 | 44.06 | C |
| ATOM | 4967 | CG | MET | P | 142 | 35.250 | 13.329 | 54.814 | 1.00 | 44.05 | C |
| ATOM | 4968 | SD | MET | P | 142 | 34.534 | 11.897 | 53.990 | 1.00 | 44.37 | S |
| ATOM | 4969 | CE | MET | P | 142 | 35.873 | 11.426 | 52.896 | 1.00 | 44.65 | C |
| ATOM | 4970 | N | TYR | P | 143 | 35.455 | 17.612 | 53.009 | 1.00 | 44.63 | N |
| ATOM | 4971 | CA | TYR | P | 143 | 35.808 | 18.603 | 51.982 | 1.00 | 44.93 | C |
| ATOM | 4972 | C | TYR | P | 143 | 35.327 | 20.004 | 52.358 | 1.00 | 45.22 | C |
| ATOM | 4973 | O | TYR | P | 143 | 36.130 | 20.868 | 52.706 | 1.00 | 45.12 | O |
| ATOM | 4974 | CB | TYR | P | 143 | 35.228 | 18.222 | 50.607 | 1.00 | 44.90 | C |
| ATOM | 4975 | CG | TYR | P | 143 | 34.978 | 16.747 | 50.395 | 1.00 | 44.52 | C |
| ATOM | 4976 | CD1 | TYR | P | 143 | 36.032 | 15.867 | 50.151 | 1.00 | 44.17 | C |
| ATOM | 4977 | CD2 | TYR | P | 143 | 33.681 | 16.234 | 50.426 | 1.00 | 44.18 | C |
| ATOM | 4978 | CE1 | TYR | P | 143 | 35.801 | 14.513 | 49.956 | 1.00 | 44.24 | C |
| ATOM | 4979 | CE2 | TYR | P | 143 | 33.440 | 14.885 | 50.234 | 1.00 | 43.73 | C |
| ATOM | 4980 | CZ | TYR | P | 143 | 34.503 | 14.032 | 49.998 | 1.00 | 44.29 | C |
| ATOM | 4981 | OH | TYR | P | 143 | 34.273 | 12.693 | 49.809 | 1.00 | 44.90 | O |
| ATOM | 4982 | N | GLY | P | 144 | 34.012 | 20.215 | 52.272 | 1.00 | 45.70 | N |
| ATOM | 4983 | CA | GLY | P | 144 | 33.389 | 21.507 | 52.569 | 1.00 | 46.05 | C |
| ATOM | 4984 | C | GLY | P | 144 | 33.687 | 22.555 | 51.519 | 1.00 | 46.26 | C |
| ATOM | 4985 | O | GLY | P | 144 | 32.817 | 22.928 | 50.729 | 1.00 | 46.18 | O |
| ATOM | 4986 | N | ASP | P | 145 | 34.934 | 23.021 | 51.521 | 1.00 | 46.60 | N |
| ATOM | 4987 | CA | ASP | P | 145 | 35.425 | 24.034 | 50.583 | 1.00 | 46.85 | C |
| ATOM | 4988 | C | ASP | P | 145 | 35.409 | 23.522 | 49.134 | 1.00 | 47.01 | C |
| ATOM | 4989 | O | ASP | P | 145 | 35.193 | 24.288 | 48.191 | 1.00 | 46.96 | O |
| ATOM | 4990 | CB | ASP | P | 145 | 36.850 | 24.445 | 50.969 | 1.00 | 46.83 | C |
| ATOM | 4991 | CG | ASP | P | 145 | 37.171 | 24.162 | 52.437 | 1.00 | 47.14 | C |
| ATOM | 4992 | OD1 | ASP | P | 145 | 36.642 | 24.870 | 53.323 | 1.00 | 47.41 | O |
| ATOM | 4993 | OD2 | ASP | P | 145 | 37.961 | 23.230 | 52.704 | 1.00 | 47.05 | O |
| ATOM | 4994 | N | LEU | P | 146 | 35.620 | 22.217 | 48.973 | 1.00 | 47.19 | N |
| ATOM | 4995 | CA | LEU | P | 146 | 35.786 | 21.611 | 47.660 | 1.00 | 47.25 | C |
| ATOM | 4996 | C | LEU | P | 146 | 34.973 | 20.319 | 47.550 | 1.00 | 47.12 | C |
| ATOM | 4997 | O | LEU | P | 146 | 35.552 | 19.237 | 47.466 | 1.00 | 47.27 | O |
| ATOM | 4998 | CB | LEU | P | 146 | 37.278 | 21.322 | 47.423 | 1.00 | 47.35 | C |
| ATOM | 4999 | CG | LEU | P | 146 | 37.893 | 21.268 | 46.019 | 1.00 | 47.65 | C |
| ATOM | 5000 | CD1 | LEU | P | 146 | 39.404 | 21.519 | 46.109 | 1.00 | 47.27 | C |
| ATOM | 5001 | CD2 | LEU | P | 146 | 37.586 | 19.953 | 45.267 | 1.00 | 47.49 | C |
| ATOM | 5002 | N | PRO | P | 147 | 33.629 | 20.419 | 47.555 | 1.00 | 46.99 | N |
| ATOM | 5003 | CA | PRO | P | 147 | 32.868 | 19.186 | 47.393 | 1.00 | 46.84 | C |
| ATOM | 5004 | C | PRO | P | 147 | 32.915 | 18.716 | 45.938 | 1.00 | 46.76 | C |
| ATOM | 5005 | O | PRO | P | 147 | 32.535 | 19.468 | 45.036 | 1.00 | 46.81 | O |
| ATOM | 5006 | CB | PRO | P | 147 | 31.450 | 19.580 | 47.812 | 1.00 | 46.78 | C |
| ATOM | 5007 | CG | PRO | P | 147 | 31.367 | 21.035 | 47.572 | 1.00 | 46.89 | C |
| ATOM | 5008 | CD | PRO | P | 147 | 32.754 | 21.599 | 47.698 | 1.00 | 47.06 | C |
| ATOM | 5009 | N | PRO | P | 148 | 33.408 | 17.484 | 45.706 | 1.00 | 46.60 | N |
| ATOM | 5010 | CA | PRO | P | 148 | 33.520 | 16.969 | 44.349 | 1.00 | 46.31 | C |
| ATOM | 5011 | C | PRO | P | 148 | 32.197 | 16.354 | 43.909 | 1.00 | 46.02 | C |
| ATOM | 5012 | O | PRO | P | 148 | 31.343 | 16.106 | 44.760 | 1.00 | 46.03 | O |
| ATOM | 5013 | CB | PRO | P | 148 | 34.596 | 15.891 | 44.482 | 1.00 | 46.39 | C |
| ATOM | 5014 | CG | PRO | P | 148 | 34.478 | 15.398 | 45.884 | 1.00 | 46.45 | C |
| ATOM | 5015 | CD | PRO | P | 148 | 33.875 | 16.507 | 46.710 | 1.00 | 46.61 | C |
| ATOM | 5016 | N | PRO | P | 149 | 32.010 | 16.130 | 42.589 | 1.00 | 45.74 | N |
| ATOM | 5017 | CA | PRO | P | 149 | 30.815 | 15.429 | 42.132 | 1.00 | 45.41 | C |
| ATOM | 5018 | C | PRO | P | 149 | 30.511 | 14.203 | 43.000 | 1.00 | 45.21 | C |
| ATOM | 5019 | O | PRO | P | 149 | 31.408 | 13.396 | 43.274 | 1.00 | 45.06 | O |
| ATOM | 5020 | CB | PRO | P | 149 | 31.189 | 15.018 | 40.710 | 1.00 | 45.42 | C |
| ATOM | 5021 | CG | PRO | P | 149 | 32.075 | 16.118 | 40.247 | 1.00 | 45.49 | C |
| ATOM | 5022 | CD | PRO | P | 149 | 32.870 | 16.532 | 41.457 | 1.00 | 45.70 | C |
| ATOM | 5023 | N | ILE | P | 150 | 29.262 | 14.100 | 43.454 | 1.00 | 44.95 | N |
| ATOM | 5024 | CA | ILE | P | 150 | 28.828 | 13.008 | 44.326 | 1.00 | 44.69 | C |
| ATOM | 5025 | C | ILE | P | 150 | 27.549 | 12.337 | 43.830 | 1.00 | 45.01 | C |
| ATOM | 5026 | O | ILE | P | 150 | 26.573 | 13.007 | 43.469 | 1.00 | 45.20 | O |
| ATOM | 5027 | CB | ILE | P | 150 | 28.653 | 13.481 | 45.789 | 1.00 | 44.51 | C |
| ATOM | 5028 | CG1 | ILE | P | 150 | 30.025 | 13.585 | 46.463 | 1.00 | 44.39 | C |
| ATOM | 5029 | CG2 | ILE | P | 150 | 27.732 | 12.529 | 46.582 | 1.00 | 43.92 | C |
| ATOM | 5030 | CD1 | ILE | P | 150 | 30.100 | 14.598 | 47.584 | 1.00 | 43.64 | C |
| ATOM | 5031 | N | MET | P | 151 | 27.574 | 11.007 | 43.802 | 1.00 | 44.92 | N |
| ATOM | 5032 | CA | MET | P | 151 | 26.380 | 10.220 | 43.577 | 1.00 | 45.01 | C |
| ATOM | 5033 | C | MET | P | 151 | 26.013 | 9.571 | 44.905 | 1.00 | 44.93 | C |
| ATOM | 5034 | O | MET | P | 151 | 26.863 | 8.951 | 45.550 | 1.00 | 45.11 | O |
| ATOM | 5035 | CB | MET | P | 151 | 26.656 | 9.143 | 42.535 | 1.00 | 44.89 | C |
| ATOM | 5036 | CG | MET | P | 151 | 25.557 | 8.979 | 41.512 | 1.00 | 45.22 | C |
| ATOM | 5037 | SD | MET | P | 151 | 25.414 | 7.287 | 40.917 | 1.00 | 45.76 | S |
| ATOM | 5038 | CE | MET | P | 151 | 23.886 | 6.787 | 41.699 | 1.00 | 46.29 | C |
| ATOM | 5039 | N | LEU | P | 152 | 24.766 | 9.737 | 45.333 | 1.00 | 44.82 | N |
| ATOM | 5040 | CA | LEU | P | 152 | 24.289 | 9.047 | 46.530 | 1.00 | 44.86 | C |
| ATOM | 5041 | C | LEU | P | 152 | 23.761 | 7.663 | 46.169 | 1.00 | 45.12 | C |
| ATOM | 5042 | O | LEU | P | 152 | 23.054 | 7.494 | 45.164 | 1.00 | 45.45 | O |
| ATOM | 5043 | CB | LEU | P | 152 | 23.225 | 9.863 | 47.273 | 1.00 | 44.66 | C |
| ATOM | 5044 | CG | LEU | P | 152 | 23.714 | 10.877 | 48.314 | 1.00 | 44.37 | C |
| ATOM | 5045 | CD1 | LEU | P | 152 | 22.569 | 11.748 | 48.790 | 1.00 | 44.19 | C |
| ATOM | 5046 | CD2 | LEU | P | 152 | 24.388 | 10.195 | 49.502 | 1.00 | 43.84 | C |
| ATOM | 5047 | N | ILE | P | 153 | 24.134 | 6.667 | 46.970 | 1.00 | 44.88 | N |
| ATOM | 5048 | CA | ILE | P | 153 | 23.664 | 5.301 | 46.763 | 1.00 | 44.50 | C |
| ATOM | 5049 | C | ILE | P | 153 | 23.306 | 4.715 | 48.118 | 1.00 | 44.57 | C |
| ATOM | 5050 | O | ILE | P | 153 | 24.174 | 4.516 | 48.976 | 1.00 | 44.44 | O |
| ATOM | 5051 | CB | ILE | P | 153 | 24.707 | 4.402 | 46.048 | 1.00 | 44.36 | C |
| ATOM | 5052 | CG1 | ILE | P | 153 | 25.207 | 5.052 | 44.754 | 1.00 | 44.21 | C |
| ATOM | 5053 | CG2 | ILE | P | 153 | 24.102 | 3.033 | 45.760 | 1.00 | 44.49 | C |
| ATOM | 5054 | CD1 | ILE | P | 153 | 26.201 | 4.216 | 43.957 | 1.00 | 44.32 | C |
| ATOM | 5055 | N | GLY | P | 154 | 22.020 | 4.447 | 48.308 | 1.00 | 44.52 | N |
| ATOM | 5056 | CA | GLY | P | 154 | 21.532 | 3.992 | 49.597 | 1.00 | 44.68 | C |
| ATOM | 5057 | C | GLY | P | 154 | 20.872 | 2.632 | 49.562 | 1.00 | 44.72 | C |
| ATOM | 5058 | O | GLY | P | 154 | 20.088 | 2.338 | 48.661 | 1.00 | 44.88 | O |
| ATOM | 5059 | N | HIS | P | 155 | 21.188 | 1.797 | 50.548 | 1.00 | 44.58 | N |
| ATOM | 5060 | CA | HIS | P | 155 | 20.523 | 0.512 | 50.674 | 1.00 | 44.52 | C |
| ATOM | 5061 | C | HIS | P | 155 | 19.531 | 0.578 | 51.817 | 1.00 | 44.41 | C |
| ATOM | 5062 | O | HIS | P | 155 | 19.843 | 1.109 | 52.876 | 1.00 | 44.32 | O |
| ATOM | 5063 | CB | HIS | P | 155 | 21.520 | −0.630 | 50.885 | 1.00 | 44.38 | C |
| ATOM | 5064 | CG | HIS | P | 155 | 20.870 | −1.968 | 51.072 | 1.00 | 44.27 | C |
| ATOM | 5065 | ND1 | HIS | P | 155 | 20.738 | −2.565 | 52.307 | 1.00 | 44.77 | N |
| ATOM | 5066 | CD2 | HIS | P | 155 | 20.302 | −2.816 | 50.184 | 1.00 | 44.24 | C |
| ATOM | 5067 | CE1 | HIS | P | 155 | 20.127 | −3.729 | 52.169 | 1.00 | 44.13 | C |
| ATOM | 5068 | NE2 | HIS | P | 155 | 19.852 | −3.906 | 50.891 | 1.00 | 43.73 | N |
| ATOM | 5069 | N | ALA | P | 156 | 18.336 | 0.036 | 51.578 | 1.00 | 44.32 | N |
| ATOM | 5070 | CA | ALA | P | 156 | 17.272 | −0.035 | 52.578 | 1.00 | 44.11 | C |
| ATOM | 5071 | C | ALA | P | 156 | 17.208 | 1.241 | 53.423 | 1.00 | 43.98 | C |
| ATOM | 5072 | O | ALA | P | 156 | 16.936 | 2.312 | 52.880 | 1.00 | 43.91 | O |
| ATOM | 5073 | CB | ALA | P | 156 | 17.424 | −1.280 | 53.436 | 1.00 | 44.17 | C |
| ATOM | 5074 | N | MET | P | 157 | 17.474 | 1.138 | 54.726 | 1.00 | 43.67 | N |
| ATOM | 5075 | CA | MET | P | 157 | 17.466 | 2.308 | 55.598 | 1.00 | 43.34 | C |
| ATOM | 5076 | C | MET | P | 157 | 18.290 | 3.436 | 54.992 | 1.00 | 43.51 | C |
| ATOM | 5077 | O | MET | P | 157 | 17.807 | 4.559 | 54.852 | 1.00 | 43.57 | O |
| ATOM | 5078 | CB | MET | P | 157 | 18.002 | 1.963 | 56.979 | 1.00 | 43.38 | C |
| ATOM | 5079 | CG | MET | P | 157 | 18.180 | 3.174 | 57.872 | 1.00 | 42.99 | C |
| ATOM | 5080 | SD | MET | P | 157 | 19.266 | 2.854 | 59.261 | 1.00 | 42.76 | S |
| ATOM | 5081 | CE | MET | P | 157 | 20.835 | 2.533 | 58.454 | 1.00 | 42.18 | C |
| ATOM | 5082 | N | GLY | P | 158 | 19.531 | 3.119 | 54.629 | 1.00 | 43.60 | N |
| ATOM | 5083 | CA | GLY | P | 158 | 20.415 | 4.044 | 53.923 | 1.00 | 43.70 | C |
| ATOM | 5084 | C | GLY | P | 158 | 19.861 | 4.564 | 52.610 | 1.00 | 43.65 | C |
| ATOM | 5085 | O | GLY | P | 158 | 20.292 | 5.611 | 52.128 | 1.00 | 43.56 | O |
| ATOM | 5086 | N | GLY | P | 159 | 18.917 | 3.827 | 52.027 | 1.00 | 43.81 | N |
| ATOM | 5087 | CA | GLY | P | 159 | 18.143 | 4.314 | 50.880 | 1.00 | 44.21 | C |
| ATOM | 5088 | C | GLY | P | 159 | 17.246 | 5.503 | 51.215 | 1.00 | 44.36 | C |
| ATOM | 5089 | O | GLY | P | 159 | 17.189 | 6.483 | 50.457 | 1.00 | 44.38 | O |
| ATOM | 5090 | N | ALA | P | 160 | 16.554 | 5.417 | 52.353 | 1.00 | 44.24 | N |
| ATOM | 5091 | CA | ALA | P | 160 | 15.672 | 6.485 | 52.820 | 1.00 | 44.46 | C |
| ATOM | 5092 | C | ALA | P | 160 | 16.454 | 7.728 | 53.252 | 1.00 | 44.80 | C |
| ATOM | 5093 | O | ALA | P | 160 | 15.995 | 8.867 | 53.083 | 1.00 | 44.80 | O |
| ATOM | 5094 | CB | ALA | P | 160 | 14.793 | 5.986 | 53.958 | 1.00 | 44.20 | C |
| ATOM | 5095 | N | ILE | P | 161 | 17.640 | 7.496 | 53.808 | 1.00 | 45.01 | N |
| ATOM | 5096 | CA | ILE | P | 161 | 18.481 | 8.568 | 54.319 | 1.00 | 44.96 | C |
| ATOM | 5097 | C | ILE | P | 161 | 19.092 | 9.322 | 53.148 | 1.00 | 44.89 | C |
| ATOM | 5098 | O | ILE | P | 161 | 19.099 | 10.554 | 53.136 | 1.00 | 44.64 | O |
| ATOM | 5099 | CB | ILE | P | 161 | 19.553 | 8.017 | 55.296 | 1.00 | 44.93 | C |
| ATOM | 5100 | CG1 | ILE | P | 161 | 18.884 | 7.319 | 56.488 | 1.00 | 45.09 | C |
| ATOM | 5101 | CG2 | ILE | P | 161 | 20.475 | 9.114 | 55.795 | 1.00 | 45.14 | C |
| ATOM | 5102 | CD1 | ILE | P | 161 | 17.671 | 8.052 | 57.076 | 1.00 | 44.43 | C |
| ATOM | 5103 | N | ALA | P | 162 | 19.568 | 8.568 | 52.157 | 1.00 | 45.09 | N |
| ATOM | 5104 | CA | ALA | P | 162 | 20.093 | 9.125 | 50.908 | 1.00 | 45.16 | C |
| ATOM | 5105 | C | ALA | P | 162 | 19.104 | 10.096 | 50.298 | 1.00 | 45.34 | C |
| ATOM | 5106 | O | ALA | P | 162 | 19.486 | 11.179 | 49.864 | 1.00 | 45.51 | O |
| ATOM | 5107 | CB | ALA | P | 162 | 20.411 | 8.017 | 49.925 | 1.00 | 44.98 | C |
| ATOM | 5108 | N | VAL | P | 163 | 17.832 | 9.704 | 50.282 | 1.00 | 45.56 | N |
| ATOM | 5109 | CA | VAL | P | 163 | 16.767 | 10.543 | 49.746 | 1.00 | 45.73 | C |
| ATOM | 5110 | C | VAL | P | 163 | 16.582 | 11.797 | 50.606 | 1.00 | 45.83 | C |
| ATOM | 5111 | O | VAL | P | 163 | 16.730 | 12.905 | 50.103 | 1.00 | 45.92 | O |
| ATOM | 5112 | CB | VAL | P | 163 | 15.449 | 9.737 | 49.538 | 1.00 | 45.89 | C |
| ATOM | 5113 | CG1 | VAL | P | 163 | 14.245 | 10.658 | 49.376 | 1.00 | 45.92 | C |
| ATOM | 5114 | CG2 | VAL | P | 163 | 15.584 | 8.817 | 48.323 | 1.00 | 45.38 | C |
| ATOM | 5115 | N | HIS | P | 164 | 16.310 | 11.622 | 51.898 | 1.00 | 46.12 | N |
| ATOM | 5116 | CA | HIS | P | 164 | 16.195 | 12.752 | 52.832 | 1.00 | 46.30 | C |
| ATOM | 5117 | C | HIS | P | 164 | 17.375 | 13.726 | 52.776 | 1.00 | 46.84 | C |
| ATOM | 5118 | O | HIS | P | 164 | 17.207 | 14.921 | 53.016 | 1.00 | 46.88 | O |
| ATOM | 5119 | CB | HIS | P | 164 | 15.997 | 12.248 | 54.258 | 1.00 | 46.04 | C |
| ATOM | 5120 | CG | HIS | P | 164 | 14.602 | 11.788 | 54.540 | 1.00 | 45.60 | C |
| ATOM | 5121 | ND1 | HIS | P | 164 | 13.719 | 12.518 | 55.305 | 1.00 | 44.64 | N |
| ATOM | 5122 | CD2 | HIS | P | 164 | 13.934 | 10.676 | 54.151 | 1.00 | 45.29 | C |
| ATOM | 5123 | CE1 | HIS | P | 164 | 12.568 | 11.874 | 55.379 | 1.00 | 44.79 | C |
| ATOM | 5124 | NE2 | HIS | P | 164 | 12.672 | 10.753 | 54.687 | 1.00 | 45.34 | N |
| ATOM | 5125 | N | THR | P | 165 | 18.560 | 13.209 | 52.457 | 1.00 | 47.56 | N |
| ATOM | 5126 | CA | THR | P | 165 | 19.757 | 14.028 | 52.274 | 1.00 | 48.17 | C |
| ATOM | 5127 | C | THR | P | 165 | 19.699 | 14.788 | 50.945 | 1.00 | 48.72 | C |
| ATOM | 5128 | O | THR | P | 165 | 19.992 | 15.990 | 50.892 | 1.00 | 48.91 | O |
| ATOM | 5129 | CB | THR | P | 165 | 21.042 | 13.167 | 52.318 | 1.00 | 48.24 | C |
| ATOM | 5130 | OG1 | THR | P | 165 | 21.054 | 12.368 | 53.509 | 1.00 | 48.27 | O |
| ATOM | 5131 | CG2 | THR | P | 165 | 22.297 | 14.048 | 52.283 | 1.00 | 48.41 | C |
| ATOM | 5132 | N | ALA | P | 166 | 19.330 | 14.080 | 49.878 | 1.00 | 49.07 | N |
| ATOM | 5133 | CA | ALA | P | 166 | 19.222 | 14.672 | 48.551 | 1.00 | 49.39 | C |
| ATOM | 5134 | C | ALA | P | 166 | 18.143 | 15.743 | 48.537 | 1.00 | 49.80 | C |
| ATOM | 5135 | O | ALA | P | 166 | 18.382 | 16.867 | 48.090 | 1.00 | 49.93 | O |
| ATOM | 5136 | CB | ALA | P | 166 | 18.915 | 13.601 | 47.519 | 1.00 | 49.37 | C |
| ATOM | 5137 | N | SER | P | 167 | 16.964 | 15.387 | 49.045 | 1.00 | 50.13 | N |
| ATOM | 5138 | CA | SER | P | 167 | 15.807 | 16.279 | 49.087 | 1.00 | 50.57 | C |
| ATOM | 5139 | C | SER | P | 167 | 16.088 | 17.615 | 49.783 | 1.00 | 50.86 | C |
| ATOM | 5140 | O | SER | P | 167 | 15.719 | 18.670 | 49.267 | 1.00 | 50.63 | O |
| ATOM | 5141 | CB | SER | P | 167 | 14.624 | 15.575 | 49.751 | 1.00 | 50.51 | C |
| ATOM | 5142 | OG | SER | P | 167 | 13.543 | 16.471 | 49.946 | 1.00 | 51.03 | O |
| ATOM | 5143 | N | SER | P | 168 | 16.740 | 17.557 | 50.946 | 1.00 | 51.45 | N |
| ATOM | 5144 | CA | SER | P | 168 | 17.110 | 18.757 | 51.712 | 1.00 | 52.01 | C |
| ATOM | 5145 | C | SER | P | 168 | 18.295 | 19.498 | 51.094 | 1.00 | 52.41 | C |
| ATOM | 5146 | O | SER | P | 168 | 18.758 | 20.510 | 51.629 | 1.00 | 52.36 | O |
| ATOM | 5147 | CB | SER | P | 168 | 17.433 | 18.387 | 53.160 | 1.00 | 51.90 | C |
| ATOM | 5148 | OG | SER | P | 168 | 16.252 | 18.080 | 53.874 | 1.00 | 52.11 | O |
| ATOM | 5149 | N | ASN | P | 169 | 18.767 | 18.980 | 49.961 | 1.00 | 52.94 | N |
| ATOM | 5150 | CA | ASN | P | 169 | 19.952 | 19.476 | 49.260 | 1.00 | 53.34 | C |
| ATOM | 5151 | C | ASN | P | 169 | 21.141 | 19.724 | 50.182 | 1.00 | 53.39 | C |
| ATOM | 5152 | O | ASN | P | 169 | 21.739 | 20.802 | 50.180 | 1.00 | 53.38 | O |
| ATOM | 5153 | CB | ASN | P | 169 | 19.631 | 20.703 | 48.406 | 1.00 | 53.45 | C |
| ATOM | 5154 | CG | ASN | P | 169 | 20.275 | 20.627 | 47.035 | 1.00 | 54.15 | C |
| ATOM | 5155 | OD1 | ASN | P | 169 | 19.729 | 20.017 | 46.107 | 1.00 | 53.50 | O |
| ATOM | 5156 | ND2 | ASN | P | 169 | 21.453 | 21.238 | 46.902 | 1.00 | 55.20 | N |
| ATOM | 5157 | N | LEU | P | 170 | 21.469 | 18.704 | 50.967 | 1.00 | 53.57 | N |
| ATOM | 5158 | CA | LEU | P | 170 | 22.568 | 18.780 | 51.911 | 1.00 | 53.86 | C |
| ATOM | 5159 | C | LEU | P | 170 | 23.909 | 18.518 | 51.232 | 1.00 | 54.11 | C |
| ATOM | 5160 | O | LEU | P | 170 | 24.955 | 18.927 | 51.740 | 1.00 | 54.27 | O |
| ATOM | 5161 | CB | LEU | P | 170 | 22.336 | 17.822 | 53.083 | 1.00 | 53.78 | C |
| ATOM | 5162 | CG | LEU | P | 170 | 21.186 | 18.193 | 54.029 | 1.00 | 53.67 | C |
| ATOM | 5163 | CD1 | LEU | P | 170 | 20.949 | 17.095 | 55.043 | 1.00 | 53.45 | C |
| ATOM | 5164 | CD2 | LEU | P | 170 | 21.444 | 19.522 | 54.739 | 1.00 | 53.79 | C |
| ATOM | 5165 | N | VAL | P | 171 | 23.874 | 17.844 | 50.086 | 1.00 | 54.34 | N |
| ATOM | 5166 | CA | VAL | P | 171 | 25.071 | 17.643 | 49.275 | 1.00 | 54.58 | C |
| ATOM | 5167 | C | VAL | P | 171 | 25.039 | 18.631 | 48.108 | 1.00 | 54.62 | C |
| ATOM | 5168 | O | VAL | P | 171 | 24.229 | 18.479 | 47.187 | 1.00 | 54.75 | O |
| ATOM | 5169 | CB | VAL | P | 171 | 25.191 | 16.193 | 48.746 | 1.00 | 54.73 | C |
| ATOM | 5170 | CG1 | VAL | P | 171 | 26.542 | 15.988 | 48.058 | 1.00 | 55.13 | C |
| ATOM | 5171 | CG2 | VAL | P | 171 | 25.013 | 15.181 | 49.874 | 1.00 | 54.84 | C |
| ATOM | 5172 | N | PRO | P | 172 | 25.923 | 19.647 | 48.141 | 1.00 | 54.57 | N |
| ATOM | 5173 | CA | PRO | P | 172 | 25.872 | 20.734 | 47.160 | 1.00 | 54.58 | C |
| ATOM | 5174 | C | PRO | P | 172 | 26.498 | 20.390 | 45.797 | 1.00 | 54.49 | C |
| ATOM | 5175 | O | PRO | P | 172 | 26.543 | 21.245 | 44.907 | 1.00 | 54.56 | O |
| ATOM | 5176 | CB | PRO | P | 172 | 26.656 | 21.857 | 47.849 | 1.00 | 54.55 | C |
| ATOM | 5177 | CG | PRO | P | 172 | 27.643 | 21.145 | 48.717 | 1.00 | 54.61 | C |
| ATOM | 5178 | CD | PRO | P | 172 | 27.027 | 19.823 | 49.104 | 1.00 | 54.64 | C |
| ATOM | 5179 | N | SER | P | 173 | 26.977 | 19.158 | 45.639 | 1.00 | 54.19 | N |
| ATOM | 5180 | CA | SER | P | 173 | 27.550 | 18.717 | 44.370 | 1.00 | 53.82 | C |
| ATOM | 5181 | C | SER | P | 173 | 26.857 | 17.458 | 43.854 | 1.00 | 53.56 | C |
| ATOM | 5182 | O | SER | P | 173 | 27.193 | 16.957 | 42.778 | 1.00 | 53.71 | O |
| ATOM | 5183 | CB | SER | P | 173 | 29.056 | 18.487 | 44.507 | 1.00 | 53.73 | C |
| ATOM | 5184 | OG | SER | P | 173 | 29.327 | 17.530 | 45.512 | 1.00 | 53.95 | O |
| ATOM | 5185 | N | LEU | P | 174 | 25.885 | 16.962 | 44.620 | 1.00 | 53.08 | N |
| ATOM | 5186 | CA | LEU | P | 174 | 25.137 | 15.750 | 44.272 | 1.00 | 52.57 | C |
| ATOM | 5187 | C | LEU | P | 174 | 24.594 | 15.789 | 42.847 | 1.00 | 52.17 | C |
| ATOM | 5188 | O | LEU | P | 174 | 23.822 | 16.682 | 42.502 | 1.00 | 52.29 | O |
| ATOM | 5189 | CB | LEU | P | 174 | 23.979 | 15.540 | 45.247 | 1.00 | 52.59 | C |
| ATOM | 5190 | CG | LEU | P | 174 | 23.152 | 14.280 | 44.999 | 1.00 | 52.50 | C |
| ATOM | 5191 | CD1 | LEU | P | 174 | 23.888 | 13.075 | 45.552 | 1.00 | 52.23 | C |
| ATOM | 5192 | CD2 | LEU | P | 174 | 21.778 | 14.410 | 45.621 | 1.00 | 51.85 | C |
| ATOM | 5193 | N | LEU | P | 175 | 24.994 | 14.818 | 42.031 | 1.00 | 51.55 | N |
| ATOM | 5194 | CA | LEU | P | 175 | 24.544 | 14.766 | 40.639 | 1.00 | 50.92 | C |
| ATOM | 5195 | C | LEU | P | 175 | 23.653 | 13.576 | 40.281 | 1.00 | 50.69 | C |
| ATOM | 5196 | O | LEU | P | 175 | 23.056 | 13.548 | 39.205 | 1.00 | 50.92 | O |
| ATOM | 5197 | CB | LEU | P | 175 | 25.715 | 14.906 | 39.652 | 1.00 | 50.92 | C |
| ATOM | 5198 | CG | LEU | P | 175 | 27.168 | 14.502 | 39.921 | 1.00 | 50.63 | C |
| ATOM | 5199 | CD1 | LEU | P | 175 | 27.371 | 12.999 | 39.964 | 1.00 | 49.97 | C |
| ATOM | 5200 | CD2 | LEU | P | 175 | 28.024 | 15.109 | 38.830 | 1.00 | 50.73 | C |
| ATOM | 5201 | N | GLY | P | 176 | 23.550 | 12.608 | 41.185 | 1.00 | 50.34 | N |
| ATOM | 5202 | CA | GLY | P | 176 | 22.704 | 11.434 | 40.967 | 1.00 | 49.87 | C |
| ATOM | 5203 | C | GLY | P | 176 | 22.269 | 10.776 | 42.263 | 1.00 | 49.52 | C |
| ATOM | 5204 | O | GLY | P | 176 | 22.827 | 11.055 | 43.324 | 1.00 | 49.65 | O |
| ATOM | 5205 | N | LEU | P | 177 | 21.268 | 9.904 | 42.181 | 1.00 | 49.08 | N |
| ATOM | 5206 | CA | LEU | P | 177 | 20.740 | 9.224 | 43.361 | 1.00 | 48.52 | C |
| ATOM | 5207 | C | LEU | P | 177 | 20.270 | 7.815 | 43.038 | 1.00 | 48.38 | C |
| ATOM | 5208 | O | LEU | P | 177 | 19.512 | 7.601 | 42.091 | 1.00 | 48.16 | O |
| ATOM | 5209 | CB | LEU | P | 177 | 19.599 | 10.032 | 43.987 | 1.00 | 48.36 | C |
| ATOM | 5210 | CG | LEU | P | 177 | 18.821 | 9.418 | 45.154 | 1.00 | 48.02 | C |
| ATOM | 5211 | CD1 | LEU | P | 177 | 19.589 | 9.525 | 46.454 | 1.00 | 47.41 | C |
| ATOM | 5212 | CD2 | LEU | P | 177 | 17.472 | 10.100 | 45.271 | 1.00 | 48.03 | C |
| ATOM | 5213 | N | CYS | P | 178 | 20.721 | 6.863 | 43.852 | 1.00 | 48.27 | N |
| ATOM | 5214 | CA | CYS | P | 178 | 20.366 | 5.456 | 43.703 | 1.00 | 47.99 | C |
| ATOM | 5215 | C | CYS | P | 178 | 19.789 | 4.882 | 44.998 | 1.00 | 47.85 | C |
| ATOM | 5216 | O | CYS | P | 178 | 20.382 | 4.986 | 46.069 | 1.00 | 47.82 | O |
| ATOM | 5217 | CB | CYS | P | 178 | 21.584 | 4.653 | 43.266 | 1.00 | 47.90 | C |
| ATOM | 5218 | SG | CYS | P | 178 | 21.236 | 2.959 | 42.844 | 1.00 | 48.09 | S |
| ATOM | 5219 | N | MET | P | 179 | 18.618 | 4.281 | 44.881 | 1.00 | 47.81 | N |
| ATOM | 5220 | CA | MET | P | 179 | 17.951 | 3.663 | 46.006 | 1.00 | 47.71 | C |
| ATOM | 5221 | C | MET | P | 179 | 17.918 | 2.160 | 45.752 | 1.00 | 47.28 | C |
| ATOM | 5222 | O | MET | P | 179 | 17.431 | 1.716 | 44.712 | 1.00 | 46.96 | O |
| ATOM | 5223 | CB | MET | P | 179 | 16.539 | 4.235 | 46.143 | 1.00 | 47.79 | C |
| ATOM | 5224 | CG | MET | P | 179 | 15.938 | 4.113 | 47.529 | 1.00 | 48.16 | C |
| ATOM | 5225 | SD | MET | P | 179 | 14.709 | 5.394 | 47.847 | 1.00 | 48.35 | S |
| ATOM | 5226 | CE | MET | P | 179 | 13.266 | 4.758 | 46.998 | 1.00 | 48.62 | C |
| ATOM | 5227 | N | ILE | P | 180 | 18.463 | 1.384 | 46.685 | 1.00 | 46.98 | N |
| ATOM | 5228 | CA | ILE | P | 180 | 18.533 | −0.060 | 46.511 | 1.00 | 46.73 | C |
| ATOM | 5229 | C | ILE | P | 180 | 17.538 | −0.771 | 47.412 | 1.00 | 46.58 | C |
| ATOM | 5230 | O | ILE | P | 180 | 17.625 | −0.686 | 48.642 | 1.00 | 46.48 | O |
| ATOM | 5231 | CB | ILE | P | 180 | 19.955 | −0.631 | 46.722 | 1.00 | 46.67 | C |
| ATOM | 5232 | CG1 | ILE | P | 180 | 20.911 | −0.099 | 45.659 | 1.00 | 46.49 | C |
| ATOM | 5233 | CG2 | ILE | P | 180 | 19.928 | −2.154 | 46.650 | 1.00 | 46.79 | C |
| ATOM | 5234 | CD1 | ILE | P | 180 | 22.308 | −0.701 | 45.732 | 1.00 | 46.78 | C |
| ATOM | 5235 | N | ASP | P | 181 | 16.591 | −1.447 | 46.761 | 1.00 | 46.32 | N |
| ATOM | 5236 | CA | ASP | P | 181 | 15.623 | −2.351 | 47.389 | 1.00 | 46.18 | C |
| ATOM | 5237 | C | ASP | P | 181 | 14.774 | −1.749 | 48.533 | 1.00 | 45.93 | C |
| ATOM | 5238 | O | ASP | P | 181 | 14.446 | −2.436 | 49.509 | 1.00 | 46.12 | O |
| ATOM | 5239 | CB | ASP | P | 181 | 16.325 | −3.648 | 47.822 | 1.00 | 46.19 | C |
| ATOM | 5240 | CG | ASP | P | 181 | 15.398 | −4.851 | 47.815 | 1.00 | 46.73 | C |
| ATOM | 5241 | OD1 | ASP | P | 181 | 14.676 | −5.070 | 46.811 | 1.00 | 47.31 | O |
| ATOM | 5242 | OD2 | ASP | P | 181 | 15.410 | −5.593 | 48.818 | 1.00 | 46.89 | O |
| ATOM | 5243 | N | VAL | P | 182 | 14.423 | −0.468 | 48.408 | 1.00 | 45.56 | N |
| ATOM | 5244 | CA | VAL | P | 182 | 13.448 | 0.156 | 49.311 | 1.00 | 45.19 | C |
| ATOM | 5245 | C | VAL | P | 182 | 12.373 | 0.871 | 48.533 | 1.00 | 44.80 | C |
| ATOM | 5246 | O | VAL | P | 182 | 12.661 | 1.725 | 47.695 | 1.00 | 44.86 | O |
| ATOM | 5247 | CB | VAL | P | 182 | 14.029 | 1.239 | 50.239 | 1.00 | 45.17 | C |
| ATOM | 5248 | CG1 | VAL | P | 182 | 13.508 | 1.034 | 51.656 | 1.00 | 45.25 | C |
| ATOM | 5249 | CG2 | VAL | P | 182 | 15.540 | 1.279 | 50.191 | 1.00 | 45.59 | C |
| ATOM | 5250 | N | VAL | P | 183 | 11.128 | 0.531 | 48.831 | 1.00 | 44.37 | N |
| ATOM | 5251 | CA | VAL | P | 183 | 9.987 | 1.268 | 48.311 | 1.00 | 43.69 | C |
| ATOM | 5252 | C | VAL | P | 183 | 9.000 | 1.466 | 49.460 | 1.00 | 43.20 | C |
| ATOM | 5253 | O | VAL | P | 183 | 8.763 | 0.552 | 50.241 | 1.00 | 43.14 | O |
| ATOM | 5254 | CB | VAL | P | 183 | 9.354 | 0.558 | 47.090 | 1.00 | 43.60 | C |
| ATOM | 5255 | CG1 | VAL | P | 183 | 8.100 | 1.281 | 46.622 | 1.00 | 43.61 | C |
| ATOM | 5256 | CG2 | VAL | P | 183 | 10.355 | 0.475 | 45.950 | 1.00 | 43.13 | C |
| ATOM | 5257 | N | GLU | P | 184 | 8.454 | 2.674 | 49.555 | 1.00 | 42.80 | N |
| ATOM | 5258 | CA | GLU | P | 184 | 7.600 | 3.090 | 50.664 | 1.00 | 42.54 | C |
| ATOM | 5259 | C | GLU | P | 184 | 6.455 | 2.150 | 50.995 | 1.00 | 42.37 | C |
| ATOM | 5260 | O | GLU | P | 184 | 6.404 | 1.595 | 52.093 | 1.00 | 42.17 | O |
| ATOM | 5261 | CB | GLU | P | 184 | 7.032 | 4.483 | 50.400 | 1.00 | 42.53 | C |
| ATOM | 5262 | CG | GLU | P | 184 | 5.954 | 4.883 | 51.386 | 1.00 | 42.19 | C |
| ATOM | 5263 | CD | GLU | P | 184 | 5.433 | 6.269 | 51.151 | 1.00 | 42.31 | C |
| ATOM | 5264 | OE1 | GLU | P | 184 | 5.809 | 6.893 | 50.136 | 1.00 | 41.46 | O |
| ATOM | 5265 | OE2 | GLU | P | 184 | 4.640 | 6.738 | 51.997 | 1.00 | 43.49 | O |
| ATOM | 5266 | N | GLY | P | 185 | 5.540 | 1.993 | 50.041 | 1.00 | 42.34 | N |
| ATOM | 5267 | CA | GLY | P | 185 | 4.320 | 1.220 | 50.238 | 1.00 | 42.40 | C |
| ATOM | 5268 | C | GLY | P | 185 | 4.556 | −0.178 | 50.762 | 1.00 | 42.45 | C |
| ATOM | 5269 | O | GLY | P | 185 | 3.846 | −0.640 | 51.655 | 1.00 | 42.38 | O |
| ATOM | 5270 | N | THR | P | 186 | 5.562 | −0.850 | 50.214 | 1.00 | 42.55 | N |
| ATOM | 5271 | CA | THR | P | 186 | 5.871 | −2.209 | 50.633 | 1.00 | 42.88 | C |
| ATOM | 5272 | C | THR | P | 186 | 6.632 | −2.259 | 51.963 | 1.00 | 43.08 | C |
| ATOM | 5273 | O | THR | P | 186 | 6.387 | −3.145 | 52.782 | 1.00 | 43.17 | O |
| ATOM | 5274 | CB | THR | P | 186 | 6.607 | −2.997 | 49.537 | 1.00 | 42.69 | C |
| ATOM | 5275 | OG1 | THR | P | 186 | 7.785 | −2.288 | 49.143 | 1.00 | 43.29 | O |
| ATOM | 5276 | CG2 | THR | P | 186 | 5.697 | −3.182 | 48.332 | 1.00 | 42.47 | C |
| ATOM | 5277 | N | ALA | P | 187 | 7.525 | −1.296 | 52.181 | 1.00 | 43.36 | N |
| ATOM | 5278 | CA | ALA | P | 187 | 8.257 | −1.188 | 53.440 | 1.00 | 43.91 | C |
| ATOM | 5279 | C | ALA | P | 187 | 7.316 | −0.915 | 54.614 | 1.00 | 44.40 | C |
| ATOM | 5280 | O | ALA | P | 187 | 7.474 | −1.481 | 55.697 | 1.00 | 44.40 | O |
| ATOM | 5281 | CB | ALA | P | 187 | 9.326 | −0.105 | 53.345 | 1.00 | 43.84 | C |
| ATOM | 5282 | N | MET | P | 188 | 6.337 | −0.043 | 54.384 | 1.00 | 45.13 | N |
| ATOM | 5283 | CA | MET | P | 188 | 5.322 | 0.281 | 55.377 | 1.00 | 45.63 | C |
| ATOM | 5284 | C | MET | P | 188 | 4.475 | −0.964 | 55.673 | 1.00 | 45.94 | C |
| ATOM | 5285 | O | MET | P | 188 | 4.130 | −1.237 | 56.826 | 1.00 | 45.88 | O |
| ATOM | 5286 | CB | MET | P | 188 | 4.456 | 1.435 | 54.873 | 1.00 | 45.60 | C |
| ATOM | 5287 | CG | MET | P | 188 | 4.121 | 2.493 | 55.925 | 1.00 | 46.77 | C |
| ATOM | 5288 | SD | MET | P | 188 | 5.436 | 3.712 | 56.204 | 1.00 | 48.69 | S |
| ATOM | 5289 | CE | MET | P | 188 | 6.153 | 3.119 | 57.724 | 1.00 | 48.22 | C |
| ATOM | 5290 | N | ASP | P | 189 | 4.173 | −1.727 | 54.622 | 1.00 | 46.36 | N |
| ATOM | 5291 | CA | ASP | P | 189 | 3.441 | −2.990 | 54.736 | 1.00 | 46.76 | C |
| ATOM | 5292 | C | ASP | P | 189 | 4.231 | −4.085 | 55.467 | 1.00 | 46.71 | C |
| ATOM | 5293 | O | ASP | P | 189 | 3.648 | −5.077 | 55.914 | 1.00 | 46.68 | O |
| ATOM | 5294 | CB | ASP | P | 189 | 3.026 | −3.496 | 53.343 | 1.00 | 47.03 | C |
| ATOM | 5295 | CG | ASP | P | 189 | 1.620 | −3.046 | 52.935 | 1.00 | 47.71 | C |
| ATOM | 5296 | OD1 | ASP | P | 189 | 0.757 | −2.870 | 53.823 | 1.00 | 48.39 | O |
| ATOM | 5297 | OD2 | ASP | P | 189 | 1.376 | −2.885 | 51.716 | 1.00 | 48.28 | O |
| ATOM | 5298 | N | ALA | P | 190 | 5.545 | −3.903 | 55.581 | 1.00 | 46.54 | N |
| ATOM | 5299 | CA | ALA | P | 190 | 6.424 | −4.931 | 56.123 | 1.00 | 46.59 | C |
| ATOM | 5300 | C | ALA | P | 190 | 6.807 | −4.701 | 57.581 | 1.00 | 46.83 | C |
| ATOM | 5301 | O | ALA | P | 190 | 7.360 | −5.590 | 58.228 | 1.00 | 46.69 | O |
| ATOM | 5302 | CB | ALA | P | 190 | 7.665 | −5.049 | 55.271 | 1.00 | 46.54 | C |
| ATOM | 5303 | N | LEU | P | 191 | 6.500 | −3.512 | 58.094 | 1.00 | 47.21 | N |
| ATOM | 5304 | CA | LEU | P | 191 | 6.937 | −3.096 | 59.428 | 1.00 | 47.58 | C |
| ATOM | 5305 | C | LEU | P | 191 | 6.865 | −4.187 | 60.503 | 1.00 | 47.83 | C |
| ATOM | 5306 | O | LEU | P | 191 | 7.873 | −4.498 | 61.140 | 1.00 | 48.03 | O |
| ATOM | 5307 | CB | LEU | P | 191 | 6.168 | −1.849 | 59.890 | 1.00 | 47.62 | C |
| ATOM | 5308 | CG | LEU | P | 191 | 6.395 | −0.482 | 59.232 | 1.00 | 47.40 | C |
| ATOM | 5309 | CD1 | LEU | P | 191 | 5.665 | 0.579 | 60.047 | 1.00 | 46.68 | C |
| ATOM | 5310 | CD2 | LEU | P | 191 | 7.882 | −0.126 | 59.079 | 1.00 | 47.10 | C |
| ATOM | 5311 | N | ASN | P | 192 | 5.680 | −4.767 | 60.688 | 1.00 | 47.94 | N |
| ATOM | 5312 | CA | ASN | P | 192 | 5.458 | −5.799 | 61.703 | 1.00 | 48.11 | C |
| ATOM | 5313 | C | ASN | P | 192 | 6.279 | −7.059 | 61.476 | 1.00 | 48.23 | C |
| ATOM | 5314 | O | ASN | P | 192 | 6.787 | −7.653 | 62.426 | 1.00 | 48.36 | O |
| ATOM | 5315 | CB | ASN | P | 192 | 3.981 | −6.174 | 61.777 | 1.00 | 48.10 | C |
| ATOM | 5316 | CG | ASN | P | 192 | 3.112 | −5.030 | 62.249 | 1.00 | 48.77 | C |
| ATOM | 5317 | OD1 | ASN | P | 192 | 2.608 | −5.050 | 63.368 | 1.00 | 49.62 | O |
| ATOM | 5318 | ND2 | ASN | P | 192 | 2.933 | −4.021 | 61.399 | 1.00 | 49.65 | N |
| ATOM | 5319 | N | SER | P | 193 | 6.407 | −7.460 | 60.215 | 1.00 | 48.38 | N |
| ATOM | 5320 | CA | SER | P | 193 | 7.108 | −8.693 | 59.868 | 1.00 | 48.43 | C |
| ATOM | 5321 | C | SER | P | 193 | 8.620 | −8.584 | 60.073 | 1.00 | 48.26 | C |
| ATOM | 5322 | O | SER | P | 193 | 9.262 | −9.570 | 60.432 | 1.00 | 48.50 | O |
| ATOM | 5323 | CB | SER | P | 193 | 6.780 | −9.134 | 58.435 | 1.00 | 48.48 | C |
| ATOM | 5324 | OG | SER | P | 193 | 7.715 | −8.609 | 57.506 | 1.00 | 49.35 | O |
| ATOM | 5325 | N | MET | P | 194 | 9.192 | −7.401 | 59.842 | 1.00 | 48.04 | N |
| ATOM | 5326 | CA | MET | P | 194 | 10.623 | −7.203 | 60.128 | 1.00 | 47.75 | C |
| ATOM | 5327 | C | MET | P | 194 | 10.896 | −6.939 | 61.602 | 1.00 | 47.26 | C |
| ATOM | 5328 | O | MET | P | 194 | 12.021 | −7.112 | 62.070 | 1.00 | 47.11 | O |
| ATOM | 5329 | CB | MET | P | 194 | 11.277 | −6.144 | 59.235 | 1.00 | 47.84 | C |
| ATOM | 5330 | CG | MET | P | 194 | 10.431 | −4.968 | 58.820 | 1.00 | 48.00 | C |
| ATOM | 5331 | SD | MET | P | 194 | 11.167 | −4.284 | 57.316 | 1.00 | 48.07 | S |
| ATOM | 5332 | CE | MET | P | 194 | 10.008 | −2.984 | 56.911 | 1.00 | 48.03 | C |
| ATOM | 5333 | N | GLN | P | 195 | 9.850 | −6.532 | 62.317 | 1.00 | 46.81 | N |
| ATOM | 5334 | CA | GLN | P | 195 | 9.843 | −6.507 | 63.773 | 1.00 | 46.26 | C |
| ATOM | 5335 | C | GLN | P | 195 | 9.991 | −7.951 | 64.265 | 1.00 | 45.76 | C |
| ATOM | 5336 | O | GLN | P | 195 | 10.780 | −8.220 | 65.168 | 1.00 | 45.45 | O |
| ATOM | 5337 | CB | GLN | P | 195 | 8.528 | −5.904 | 64.271 | 1.00 | 46.39 | C |
| ATOM | 5338 | CG | GLN | P | 195 | 8.671 | −4.706 | 65.189 | 1.00 | 47.05 | C |
| ATOM | 5339 | CD | GLN | P | 195 | 8.698 | −5.072 | 66.663 | 1.00 | 48.43 | C |
| ATOM | 5340 | OE1 | GLN | P | 195 | 7.720 | −4.855 | 67.385 | 1.00 | 48.87 | O |
| ATOM | 5341 | NE2 | GLN | P | 195 | 9.819 | −5.623 | 67.120 | 1.00 | 49.13 | N |
| ATOM | 5342 | N | ASN | P | 196 | 9.246 | −8.869 | 63.641 | 1.00 | 45.31 | N |
| ATOM | 5343 | CA | ASN | P | 196 | 9.317 | −10.305 | 63.940 | 1.00 | 45.03 | C |
| ATOM | 5344 | C | ASN | P | 196 | 10.676 | −10.942 | 63.642 | 1.00 | 44.93 | C |
| ATOM | 5345 | O | ASN | P | 196 | 11.188 | −11.728 | 64.442 | 1.00 | 44.72 | O |
| ATOM | 5346 | CB | ASN | P | 196 | 8.227 | −11.074 | 63.186 | 1.00 | 44.93 | C |
| ATOM | 5347 | CG | ASN | P | 196 | 6.841 | −10.832 | 63.739 | 1.00 | 45.06 | C |
| ATOM | 5348 | OD1 | ASN | P | 196 | 6.656 | −10.105 | 64.712 | 1.00 | 45.24 | O |
| ATOM | 5349 | ND2 | ASN | P | 196 | 5.850 | −11.443 | 63.109 | 1.00 | 45.47 | N |
| ATOM | 5350 | N | PHE | P | 197 | 11.250 | −10.619 | 62.485 | 1.00 | 44.95 | N |
| ATOM | 5351 | CA | PHE | P | 197 | 12.550 | −11.163 | 62.122 | 1.00 | 45.13 | C |
| ATOM | 5352 | C | PHE | P | 197 | 13.594 | −10.852 | 63.187 | 1.00 | 44.99 | C |
| ATOM | 5353 | O | PHE | P | 197 | 14.301 | −11.746 | 63.654 | 1.00 | 45.02 | O |
| ATOM | 5354 | CB | PHE | P | 197 | 13.039 | −10.636 | 60.766 | 1.00 | 45.40 | C |
| ATOM | 5355 | CG | PHE | P | 197 | 14.514 | −10.869 | 60.535 | 1.00 | 45.57 | C |
| ATOM | 5356 | CD1 | PHE | P | 197 | 14.988 | −12.134 | 60.186 | 1.00 | 46.09 | C |
| ATOM | 5357 | CD2 | PHE | P | 197 | 15.430 | −9.836 | 60.707 | 1.00 | 45.75 | C |
| ATOM | 5358 | CE1 | PHE | P | 197 | 16.352 | −12.360 | 59.997 | 1.00 | 46.27 | C |
| ATOM | 5359 | CE2 | PHE | P | 197 | 16.794 | −10.049 | 60.517 | 1.00 | 46.07 | C |
| ATOM | 5360 | CZ | PHE | P | 197 | 17.257 | −11.312 | 60.163 | 1.00 | 46.06 | C |
| ATOM | 5361 | N | LEU | P | 198 | 13.682 | −9.574 | 63.550 | 1.00 | 44.80 | N |
| ATOM | 5362 | CA | LEU | P | 198 | 14.699 | −9.092 | 64.473 | 1.00 | 44.47 | C |
| ATOM | 5363 | C | LEU | P | 198 | 14.649 | −9.802 | 65.811 | 1.00 | 44.09 | C |
| ATOM | 5364 | O | LEU | P | 198 | 15.654 | −10.350 | 66.248 | 1.00 | 44.14 | O |
| ATOM | 5365 | CB | LEU | P | 198 | 14.591 | −7.577 | 64.671 | 1.00 | 44.50 | C |
| ATOM | 5366 | CG | LEU | P | 198 | 15.061 | −6.665 | 63.537 | 1.00 | 44.85 | C |
| ATOM | 5367 | CD1 | LEU | P | 198 | 15.072 | −5.226 | 64.037 | 1.00 | 45.36 | C |
| ATOM | 5368 | CD2 | LEU | P | 198 | 16.439 | −7.061 | 62.982 | 1.00 | 44.90 | C |
| ATOM | 5369 | N | ARG | P | 199 | 13.487 | −9.813 | 66.457 | 1.00 | 43.77 | N |
| ATOM | 5370 | CA | ARG | P | 199 | 13.374 | −10.521 | 67.731 | 1.00 | 43.51 | C |
| ATOM | 5371 | C | ARG | P | 199 | 13.227 | −12.029 | 67.561 | 1.00 | 43.18 | C |
| ATOM | 5372 | O | ARG | P | 199 | 12.810 | −12.728 | 68.478 | 1.00 | 43.46 | O |
| ATOM | 5373 | CB | ARG | P | 199 | 12.315 | −9.910 | 68.664 | 1.00 | 43.51 | C |
| ATOM | 5374 | CG | ARG | P | 199 | 10.918 | −9.713 | 68.107 | 1.00 | 43.64 | C |
| ATOM | 5375 | CD | ARG | P | 199 | 10.098 | −8.877 | 69.089 | 1.00 | 43.74 | C |
| ATOM | 5376 | NE | ARG | P | 199 | 8.660 | −8.955 | 68.843 | 1.00 | 45.33 | N |
| ATOM | 5377 | CZ | ARG | P | 199 | 7.722 | −8.815 | 69.782 | 1.00 | 46.05 | C |
| ATOM | 5378 | NH1 | ARG | P | 199 | 8.059 | −8.601 | 71.052 | 1.00 | 46.22 | N |
| ATOM | 5379 | NH2 | ARG | P | 199 | 6.437 | −8.901 | 69.455 | 1.00 | 45.82 | N |
| ATOM | 5380 | N | GLY | P | 200 | 13.600 | −12.519 | 66.381 | 1.00 | 42.76 | N |
| ATOM | 5381 | CA | GLY | P | 200 | 13.756 | −13.946 | 66.123 | 1.00 | 42.07 | C |
| ATOM | 5382 | C | GLY | P | 200 | 15.220 | −14.309 | 65.949 | 1.00 | 41.71 | C |
| ATOM | 5383 | O | GLY | P | 200 | 15.616 | −15.437 | 66.239 | 1.00 | 41.71 | O |
| ATOM | 5384 | N | ARG | P | 201 | 16.020 | −13.346 | 65.486 | 1.00 | 41.40 | N |
| ATOM | 5385 | CA | ARG | P | 201 | 17.463 | −13.516 | 65.279 | 1.00 | 41.31 | C |
| ATOM | 5386 | C | ARG | P | 201 | 18.138 | −14.134 | 66.485 | 1.00 | 40.88 | C |
| ATOM | 5387 | O | ARG | P | 201 | 17.673 | −13.950 | 67.604 | 1.00 | 41.01 | O |
| ATOM | 5388 | CB | ARG | P | 201 | 18.148 | −12.168 | 65.048 | 1.00 | 41.49 | C |
| ATOM | 5389 | CG | ARG | P | 201 | 17.718 | −11.400 | 63.828 | 1.00 | 42.75 | C |
| ATOM | 5390 | CD | ARG | P | 201 | 18.750 | −10.330 | 63.483 | 1.00 | 44.99 | C |
| ATOM | 5391 | NE | ARG | P | 201 | 19.110 | −9.468 | 64.617 | 1.00 | 45.69 | N |
| ATOM | 5392 | CZ | ARG | P | 201 | 19.741 | −8.300 | 64.492 | 1.00 | 46.09 | C |
| ATOM | 5393 | NH1 | ARG | P | 201 | 20.081 | −7.850 | 63.286 | 1.00 | 46.63 | N |
| ATOM | 5394 | NH2 | ARG | P | 201 | 20.034 | −7.578 | 65.565 | 1.00 | 45.06 | N |
| ATOM | 5395 | N | PRO | P | 202 | 19.251 | −14.859 | 66.269 | 1.00 | 40.57 | N |
| ATOM | 5396 | CA | PRO | P | 202 | 20.062 | −15.267 | 67.413 | 1.00 | 40.42 | C |
| ATOM | 5397 | C | PRO | P | 202 | 20.540 | −14.040 | 68.196 | 1.00 | 40.45 | C |
| ATOM | 5398 | O | PRO | P | 202 | 20.791 | −12.988 | 67.600 | 1.00 | 40.18 | O |
| ATOM | 5399 | CB | PRO | P | 202 | 21.244 | −15.984 | 66.761 | 1.00 | 40.41 | C |
| ATOM | 5400 | CG | PRO | P | 202 | 20.723 | −16.437 | 65.436 | 1.00 | 40.09 | C |
| ATOM | 5401 | CD | PRO | P | 202 | 19.799 | −15.359 | 64.996 | 1.00 | 40.25 | C |
| ATOM | 5402 | N | LYS | P | 203 | 20.636 | −14.171 | 69.518 | 1.00 | 40.56 | N |
| ATOM | 5403 | CA | LYS | P | 203 | 21.057 | −13.060 | 70.374 | 1.00 | 40.74 | C |
| ATOM | 5404 | C | LYS | P | 203 | 22.572 | −12.850 | 70.372 | 1.00 | 40.71 | C |
| ATOM | 5405 | O | LYS | P | 203 | 23.040 | −11.711 | 70.385 | 1.00 | 40.90 | O |
| ATOM | 5406 | CB | LYS | P | 203 | 20.567 | −13.255 | 71.808 | 1.00 | 40.74 | C |
| ATOM | 5407 | CG | LYS | P | 203 | 19.078 | −13.050 | 72.012 | 1.00 | 40.92 | C |
| ATOM | 5408 | CD | LYS | P | 203 | 18.713 | −13.195 | 73.493 | 1.00 | 41.30 | C |
| ATOM | 5409 | CE | LYS | P | 203 | 17.212 | −13.412 | 73.700 | 1.00 | 42.86 | C |
| ATOM | 5410 | NZ | LYS | P | 203 | 16.368 | −12.435 | 72.926 | 1.00 | 43.26 | N |
| ATOM | 5411 | N | THR | P | 204 | 23.330 | −13.945 | 70.376 | 1.00 | 40.73 | N |
| ATOM | 5412 | CA | THR | P | 204 | 24.798 | −13.894 | 70.398 | 1.00 | 40.58 | C |
| ATOM | 5413 | C | THR | P | 204 | 25.396 | −14.882 | 69.400 | 1.00 | 40.72 | C |
| ATOM | 5414 | O | THR | P | 204 | 24.681 | −15.726 | 68.859 | 1.00 | 40.96 | O |
| ATOM | 5415 | CB | THR | P | 204 | 25.370 | −14.189 | 71.810 | 1.00 | 40.36 | C |
| ATOM | 5416 | OG1 | THR | P | 204 | 24.916 | −15.469 | 72.261 | 1.00 | 39.92 | O |
| ATOM | 5417 | CG2 | THR | P | 204 | 24.940 | −13.128 | 72.807 | 1.00 | 40.24 | C |
| ATOM | 5418 | N | PHE | P | 205 | 26.700 | −14.769 | 69.153 | 1.00 | 40.77 | N |
| ATOM | 5419 | CA | PHE | P | 205 | 27.421 | −15.727 | 68.310 | 1.00 | 40.81 | C |
| ATOM | 5420 | C | PHE | P | 205 | 28.672 | −16.236 | 69.001 | 1.00 | 40.85 | C |
| ATOM | 5421 | O | PHE | P | 205 | 29.437 | −15.444 | 69.563 | 1.00 | 40.71 | O |
| ATOM | 5422 | CB | PHE | P | 205 | 27.802 | −15.111 | 66.960 | 1.00 | 40.86 | C |
| ATOM | 5423 | CG | PHE | P | 205 | 26.634 | −14.835 | 66.076 | 1.00 | 40.74 | C |
| ATOM | 5424 | CD1 | PHE | P | 205 | 25.956 | −15.879 | 65.453 | 1.00 | 41.15 | C |
| ATOM | 5425 | CD2 | PHE | P | 205 | 26.206 | −13.532 | 65.866 | 1.00 | 40.90 | C |
| ATOM | 5426 | CE1 | PHE | P | 205 | 24.858 | −15.629 | 64.632 | 1.00 | 41.89 | C |
| ATOM | 5427 | CE2 | PHE | P | 205 | 25.111 | −13.264 | 65.043 | 1.00 | 41.69 | C |
| ATOM | 5428 | CZ | PHE | P | 205 | 24.434 | −14.313 | 64.423 | 1.00 | 41.77 | C |
| ATOM | 5429 | N | LYS | P | 206 | 28.875 | −17.554 | 68.946 | 1.00 | 40.94 | N |
| ATOM | 5430 | CA | LYS | P | 206 | 30.054 | −18.196 | 69.542 | 1.00 | 41.16 | C |
| ATOM | 5431 | C | LYS | P | 206 | 31.350 | −17.590 | 68.996 | 1.00 | 41.08 | C |
| ATOM | 5432 | O | LYS | P | 206 | 32.287 | −17.333 | 69.748 | 1.00 | 41.05 | O |
| ATOM | 5433 | CB | LYS | P | 206 | 30.033 | −19.714 | 69.314 | 1.00 | 41.15 | C |
| ATOM | 5434 | CG | LYS | P | 206 | 28.975 | −20.486 | 70.109 | 1.00 | 41.44 | C |
| ATOM | 5435 | CD | LYS | P | 206 | 28.850 | −21.921 | 69.594 | 1.00 | 41.54 | C |
| ATOM | 5436 | CE | LYS | P | 206 | 28.418 | −22.901 | 70.688 | 1.00 | 42.32 | C |
| ATOM | 5437 | NZ | LYS | P | 206 | 26.944 | −22.907 | 70.907 | 1.00 | 42.69 | N |
| ATOM | 5438 | N | SER | P | 207 | 31.382 | −17.353 | 67.686 | 1.00 | 41.12 | N |
| ATOM | 5439 | CA | SER | P | 207 | 32.526 | −16.736 | 67.027 | 1.00 | 41.12 | C |
| ATOM | 5440 | C | SER | P | 207 | 32.101 | −16.006 | 65.758 | 1.00 | 41.19 | C |
| ATOM | 5441 | O | SER | P | 207 | 31.037 | −16.271 | 65.198 | 1.00 | 41.17 | O |
| ATOM | 5442 | CB | SER | P | 207 | 33.574 | −17.788 | 66.675 | 1.00 | 41.11 | C |
| ATOM | 5443 | OG | SER | P | 207 | 33.123 | −18.601 | 65.608 | 1.00 | 41.28 | O |
| ATOM | 5444 | N | LEU | P | 208 | 32.959 | −15.092 | 65.317 | 1.00 | 41.29 | N |
| ATOM | 5445 | CA | LEU | P | 208 | 32.795 | −14.344 | 64.076 | 1.00 | 41.32 | C |
| ATOM | 5446 | C | LEU | P | 208 | 32.558 | −15.266 | 62.877 | 1.00 | 41.43 | C |
| ATOM | 5447 | O | LEU | P | 208 | 31.806 | −14.926 | 61.959 | 1.00 | 41.40 | O |
| ATOM | 5448 | CB | LEU | P | 208 | 34.043 | −13.492 | 63.857 | 1.00 | 41.21 | C |
| ATOM | 5449 | CG | LEU | P | 208 | 34.057 | −12.250 | 62.973 | 1.00 | 41.48 | C |
| ATOM | 5450 | CD1 | LEU | P | 208 | 32.803 | −11.394 | 63.126 | 1.00 | 41.37 | C |
| ATOM | 5451 | CD2 | LEU | P | 208 | 35.308 | −11.448 | 63.312 | 1.00 | 41.46 | C |
| ATOM | 5452 | N | GLU | P | 209 | 33.199 | −16.435 | 62.897 | 1.00 | 41.58 | N |
| ATOM | 5453 | CA | GLU | P | 209 | 32.968 | −17.476 | 61.894 | 1.00 | 41.71 | C |
| ATOM | 5454 | C | GLU | P | 209 | 31.503 | −17.930 | 61.858 | 1.00 | 41.68 | C |
| ATOM | 5455 | O | GLU | P | 209 | 30.907 | −18.023 | 60.784 | 1.00 | 41.68 | O |
| ATOM | 5456 | CB | GLU | P | 209 | 33.879 | −18.676 | 62.146 | 1.00 | 41.75 | C |
| ATOM | 5457 | CG | GLU | P | 209 | 35.294 | −18.516 | 61.612 | 1.00 | 42.17 | C |
| ATOM | 5458 | CD | GLU | P | 209 | 36.183 | −17.624 | 62.475 | 1.00 | 42.64 | C |
| ATOM | 5459 | OE1 | GLU | P | 209 | 35.856 | −17.381 | 63.664 | 1.00 | 42.30 | O |
| ATOM | 5460 | OE2 | GLU | P | 209 | 37.226 | −17.175 | 61.951 | 1.00 | 42.51 | O |
| ATOM | 5461 | N | ASN | P | 210 | 30.934 | −18.200 | 63.032 | 1.00 | 41.59 | N |
| ATOM | 5462 | CA | ASN | P | 210 | 29.530 | −18.602 | 63.144 | 1.00 | 41.40 | C |
| ATOM | 5463 | C | ASN | P | 210 | 28.575 | −17.516 | 62.682 | 1.00 | 41.08 | C |
| ATOM | 5464 | O | ASN | P | 210 | 27.525 | −17.820 | 62.117 | 1.00 | 41.35 | O |
| ATOM | 5465 | CB | ASN | P | 210 | 29.179 | −19.018 | 64.575 | 1.00 | 41.54 | C |
| ATOM | 5466 | CG | ASN | P | 210 | 29.931 | −20.257 | 65.035 | 1.00 | 41.77 | C |
| ATOM | 5467 | OD1 | ASN | P | 210 | 29.955 | −20.564 | 66.223 | 1.00 | 42.29 | O |
| ATOM | 5468 | ND2 | ASN | P | 210 | 30.544 | −20.976 | 64.098 | 1.00 | 42.51 | N |
| ATOM | 5469 | N | ALA | P | 211 | 28.936 | −16.257 | 62.921 | 1.00 | 40.52 | N |
| ATOM | 5470 | CA | ALA | P | 211 | 28.126 | −15.135 | 62.451 | 1.00 | 40.16 | C |
| ATOM | 5471 | C | ALA | P | 211 | 28.092 | −15.120 | 60.926 | 1.00 | 39.99 | C |
| ATOM | 5472 | O | ALA | P | 211 | 27.019 | −15.001 | 60.330 | 1.00 | 40.21 | O |
| ATOM | 5473 | CB | ALA | P | 211 | 28.645 | −13.819 | 62.998 | 1.00 | 40.03 | C |
| ATOM | 5474 | N | ILE | P | 212 | 29.268 | −15.266 | 60.307 | 1.00 | 39.66 | N |
| ATOM | 5475 | CA | ILE | P | 212 | 29.404 | −15.374 | 58.846 | 1.00 | 39.00 | C |
| ATOM | 5476 | C | ILE | P | 212 | 28.609 | −16.570 | 58.299 | 1.00 | 38.80 | C |
| ATOM | 5477 | O | ILE | P | 212 | 27.862 | −16.442 | 57.322 | 1.00 | 38.73 | O |
| ATOM | 5478 | CB | ILE | P | 212 | 30.905 | −15.439 | 58.424 | 1.00 | 38.89 | C |
| ATOM | 5479 | CG1 | ILE | P | 212 | 31.546 | −14.055 | 58.558 | 1.00 | 38.63 | C |
| ATOM | 5480 | CG2 | ILE | P | 212 | 31.070 | −15.964 | 56.997 | 1.00 | 38.51 | C |
| ATOM | 5481 | CD1 | ILE | P | 212 | 33.060 | −14.062 | 58.604 | 1.00 | 38.24 | C |
| ATOM | 5482 | N | GLU | P | 213 | 28.758 | −17.719 | 58.953 | 1.00 | 38.42 | N |
| ATOM | 5483 | CA | GLU | P | 213 | 28.062 | −18.936 | 58.560 | 1.00 | 38.15 | C |
| ATOM | 5484 | C | GLU | P | 213 | 26.552 | −18.751 | 58.639 | 1.00 | 37.80 | C |
| ATOM | 5485 | O | GLU | P | 213 | 25.828 | −19.090 | 57.691 | 1.00 | 38.05 | O |
| ATOM | 5486 | CB | GLU | P | 213 | 28.496 | −20.101 | 59.445 | 1.00 | 38.25 | C |
| ATOM | 5487 | CG | GLU | P | 213 | 28.002 | −21.452 | 58.977 | 1.00 | 39.18 | C |
| ATOM | 5488 | CD | GLU | P | 213 | 28.592 | −22.599 | 59.775 | 1.00 | 40.82 | C |
| ATOM | 5489 | OE1 | GLU | P | 213 | 29.391 | −22.344 | 60.704 | 1.00 | 41.77 | O |
| ATOM | 5490 | OE2 | GLU | P | 213 | 28.255 | −23.762 | 59.472 | 1.00 | 41.53 | O |
| ATOM | 5491 | N | TRP | P | 214 | 26.090 | −18.195 | 59.761 | 1.00 | 36.99 | N |
| ATOM | 5492 | CA | TRP | P | 214 | 24.667 | −17.968 | 59.988 | 1.00 | 36.09 | C |
| ATOM | 5493 | C | TRP | P | 214 | 24.043 | −17.054 | 58.934 | 1.00 | 36.11 | C |
| ATOM | 5494 | O | TRP | P | 214 | 22.905 | −17.269 | 58.530 | 1.00 | 35.96 | O |
| ATOM | 5495 | CB | TRP | P | 214 | 24.412 | −17.415 | 61.397 | 1.00 | 35.50 | C |
| ATOM | 5496 | CG | TRP | P | 214 | 23.006 | −16.917 | 61.567 | 1.00 | 34.63 | C |
| ATOM | 5497 | CD1 | TRP | P | 214 | 21.915 | −17.653 | 61.916 | 1.00 | 33.73 | C |
| ATOM | 5498 | CD2 | TRP | P | 214 | 22.538 | −15.582 | 61.351 | 1.00 | 33.83 | C |
| ATOM | 5499 | NE1 | TRP | P | 214 | 20.797 | −16.862 | 61.939 | 1.00 | 33.46 | N |
| ATOM | 5500 | CE2 | TRP | P | 214 | 21.150 | −15.584 | 61.598 | 1.00 | 33.63 | C |
| ATOM | 5501 | CE3 | TRP | P | 214 | 23.160 | −14.383 | 60.974 | 1.00 | 33.54 | C |
| ATOM | 5502 | CZ2 | TRP | P | 214 | 20.367 | −14.431 | 61.486 | 1.00 | 34.02 | C |
| ATOM | 5503 | CZ3 | TRP | P | 214 | 22.385 | −13.240 | 60.858 | 1.00 | 33.92 | C |
| ATOM | 5504 | CH2 | TRP | P | 214 | 21.001 | −13.272 | 61.118 | 1.00 | 34.34 | C |
| ATOM | 5505 | N | SER | P | 215 | 24.785 | −16.039 | 58.498 | 1.00 | 36.31 | N |
| ATOM | 5506 | CA | SER | P | 215 | 24.274 | −15.068 | 57.527 | 1.00 | 36.72 | C |
| ATOM | 5507 | C | SER | P | 215 | 23.991 | −15.697 | 56.156 | 1.00 | 36.89 | C |
| ATOM | 5508 | O | SER | P | 215 | 23.017 | −15.337 | 55.479 | 1.00 | 36.46 | O |
| ATOM | 5509 | CB | SER | P | 215 | 25.236 | −13.887 | 57.385 | 1.00 | 36.71 | C |
| ATOM | 5510 | OG | SER | P | 215 | 26.504 | −14.322 | 56.934 | 1.00 | 37.26 | O |
| ATOM | 5511 | N | VAL | P | 216 | 24.842 | −16.636 | 55.753 | 1.00 | 37.18 | N |
| ATOM | 5512 | CA | VAL | P | 216 | 24.607 | −17.348 | 54.510 | 1.00 | 37.67 | C |
| ATOM | 5513 | C | VAL | P | 216 | 23.520 | −18.391 | 54.722 | 1.00 | 37.93 | C |
| ATOM | 5514 | O | VAL | P | 216 | 22.581 | −18.461 | 53.931 | 1.00 | 38.13 | O |
| ATOM | 5515 | CB | VAL | P | 216 | 25.891 | −17.972 | 53.902 | 1.00 | 37.76 | C |
| ATOM | 5516 | CG1 | VAL | P | 216 | 25.555 | −18.735 | 52.625 | 1.00 | 38.13 | C |
| ATOM | 5517 | CG2 | VAL | P | 216 | 26.917 | −16.892 | 53.589 | 1.00 | 37.50 | C |
| ATOM | 5518 | N | LYS | P | 217 | 23.631 | −19.178 | 55.793 | 1.00 | 38.14 | N |
| ATOM | 5519 | CA | LYS | P | 217 | 22.638 | −20.224 | 56.070 | 1.00 | 38.46 | C |
| ATOM | 5520 | C | LYS | P | 217 | 21.221 | −19.698 | 56.307 | 1.00 | 38.66 | C |
| ATOM | 5521 | O | LYS | P | 217 | 20.256 | −20.312 | 55.861 | 1.00 | 38.85 | O |
| ATOM | 5522 | CB | LYS | P | 217 | 23.078 | −21.131 | 57.217 | 1.00 | 38.48 | C |
| ATOM | 5523 | CG | LYS | P | 217 | 24.057 | −22.221 | 56.798 | 1.00 | 38.72 | C |
| ATOM | 5524 | CD | LYS | P | 217 | 24.266 | −23.237 | 57.913 | 1.00 | 40.12 | C |
| ATOM | 5525 | CE | LYS | P | 217 | 25.491 | −24.114 | 57.654 | 1.00 | 40.86 | C |
| ATOM | 5526 | NZ | LYS | P | 217 | 25.699 | −25.151 | 58.713 | 1.00 | 40.98 | N |
| ATOM | 5527 | N | SER | P | 218 | 21.095 | −18.562 | 56.988 | 1.00 | 38.93 | N |
| ATOM | 5528 | CA | SER | P | 218 | 19.786 | −17.941 | 57.188 | 1.00 | 39.42 | C |
| ATOM | 5529 | C | SER | P | 218 | 19.253 | −17.286 | 55.908 | 1.00 | 39.80 | C |
| ATOM | 5530 | O | SER | P | 218 | 18.045 | −17.143 | 55.734 | 1.00 | 39.93 | O |
| ATOM | 5531 | CB | SER | P | 218 | 19.825 | −16.921 | 58.329 | 1.00 | 39.40 | C |
| ATOM | 5532 | OG | SER | P | 218 | 20.478 | −15.728 | 57.929 | 1.00 | 39.52 | O |
| ATOM | 5533 | N | GLY | P | 219 | 20.156 | −16.887 | 55.020 | 1.00 | 40.19 | N |
| ATOM | 5534 | CA | GLY | P | 219 | 19.766 | −16.279 | 53.761 | 1.00 | 40.69 | C |
| ATOM | 5535 | C | GLY | P | 219 | 19.829 | −14.764 | 53.775 | 1.00 | 41.26 | C |
| ATOM | 5536 | O | GLY | P | 219 | 19.382 | −14.118 | 52.830 | 1.00 | 41.28 | O |
| ATOM | 5537 | N | GLN | P | 220 | 20.381 | −14.191 | 54.841 | 1.00 | 41.69 | N |
| ATOM | 5538 | CA | GLN | P | 220 | 20.579 | −12.748 | 54.906 | 1.00 | 42.36 | C |
| ATOM | 5539 | C | GLN | P | 220 | 21.489 | −12.264 | 53.776 | 1.00 | 42.33 | C |
| ATOM | 5540 | O | GLN | P | 220 | 21.195 | −11.248 | 53.137 | 1.00 | 42.70 | O |
| ATOM | 5541 | CB | GLN | P | 220 | 21.143 | −12.328 | 56.266 | 1.00 | 42.39 | C |
| ATOM | 5542 | CG | GLN | P | 220 | 21.483 | −10.835 | 56.369 | 1.00 | 43.14 | C |
| ATOM | 5543 | CD | GLN | P | 220 | 22.299 | −10.489 | 57.609 | 1.00 | 43.33 | C |
| ATOM | 5544 | OE1 | GLN | P | 220 | 22.015 | −9.505 | 58.301 | 1.00 | 44.92 | O |
| ATOM | 5545 | NE2 | GLN | P | 220 | 23.314 | −11.296 | 57.896 | 1.00 | 44.18 | N |
| ATOM | 5546 | N | ILE | P | 221 | 22.583 | −12.986 | 53.534 | 1.00 | 42.26 | N |
| ATOM | 5547 | CA | ILE | P | 221 | 23.516 | −12.659 | 52.445 | 1.00 | 42.18 | C |
| ATOM | 5548 | C | ILE | P | 221 | 23.778 | −13.904 | 51.603 | 1.00 | 42.18 | C |
| ATOM | 5549 | O | ILE | P | 221 | 24.193 | −14.934 | 52.127 | 1.00 | 42.11 | O |
| ATOM | 5550 | CB | ILE | P | 221 | 24.861 | −12.092 | 52.979 | 1.00 | 42.20 | C |
| ATOM | 5551 | CG1 | ILE | P | 221 | 24.646 | −10.775 | 53.730 | 1.00 | 41.79 | C |
| ATOM | 5552 | CG2 | ILE | P | 221 | 25.857 | −11.879 | 51.838 | 1.00 | 42.36 | C |
| ATOM | 5553 | CD1 | ILE | P | 221 | 25.662 | −10.525 | 54.830 | 1.00 | 41.24 | C |
| ATOM | 5554 | N | ARG | P | 222 | 23.548 | −13.791 | 50.298 | 1.00 | 42.45 | N |
| ATOM | 5555 | CA | ARG | P | 222 | 23.661 | −14.924 | 49.371 | 1.00 | 42.72 | C |
| ATOM | 5556 | C | ARG | P | 222 | 25.053 | −15.056 | 48.743 | 1.00 | 42.80 | C |
| ATOM | 5557 | O | ARG | P | 222 | 25.289 | −15.947 | 47.928 | 1.00 | 42.95 | O |
| ATOM | 5558 | CB | ARG | P | 222 | 22.593 | −14.838 | 48.273 | 1.00 | 42.79 | C |
| ATOM | 5559 | CG | ARG | P | 222 | 21.215 | −14.405 | 48.777 | 1.00 | 43.97 | C |
| ATOM | 5560 | CD | ARG | P | 222 | 20.130 | −15.369 | 48.359 | 1.00 | 45.45 | C |
| ATOM | 5561 | NE | ARG | P | 222 | 19.621 | −15.093 | 47.022 | 1.00 | 46.63 | N |
| ATOM | 5562 | CZ | ARG | P | 222 | 18.345 | −14.840 | 46.739 | 1.00 | 47.58 | C |
| ATOM | 5563 | NH1 | ARG | P | 222 | 17.420 | −14.826 | 47.699 | 1.00 | 46.85 | N |
| ATOM | 5564 | NH2 | ARG | P | 222 | 17.993 | −14.607 | 45.481 | 1.00 | 48.21 | N |
| ATOM | 5565 | N | ASN | P | 223 | 25.970 | −14.172 | 49.132 | 1.00 | 42.77 | N |
| ATOM | 5566 | CA | ASN | P | 223 | 27.325 | −14.174 | 48.595 | 1.00 | 42.59 | C |
| ATOM | 5567 | C | ASN | P | 223 | 28.349 | −14.318 | 49.708 | 1.00 | 42.76 | C |
| ATOM | 5568 | O | ASN | P | 223 | 28.570 | −13.394 | 50.495 | 1.00 | 42.86 | O |
| ATOM | 5569 | CB | ASN | P | 223 | 27.571 | −12.901 | 47.776 | 1.00 | 42.52 | C |
| ATOM | 5570 | CG | ASN | P | 223 | 29.001 | −12.780 | 47.268 | 1.00 | 41.65 | C |
| ATOM | 5571 | OD1 | ASN | P | 223 | 29.752 | −13.756 | 47.220 | 1.00 | 40.48 | O |
| ATOM | 5572 | ND2 | ASN | P | 223 | 29.379 | −11.568 | 46.881 | 1.00 | 41.06 | N |
| ATOM | 5573 | N | LEU | P | 224 | 28.978 | −15.485 | 49.756 | 1.00 | 42.87 | N |
| ATOM | 5574 | CA | LEU | P | 224 | 29.903 | −15.825 | 50.829 | 1.00 | 42.98 | C |
| ATOM | 5575 | C | LEU | P | 224 | 31.111 | −14.886 | 50.883 | 1.00 | 43.06 | C |
| ATOM | 5576 | O | LEU | P | 224 | 31.558 | −14.511 | 51.969 | 1.00 | 43.02 | O |
| ATOM | 5577 | CB | LEU | P | 224 | 30.328 | −17.294 | 50.715 | 1.00 | 42.96 | C |
| ATOM | 5578 | CG | LEU | P | 224 | 31.052 | −17.959 | 51.891 | 1.00 | 43.32 | C |
| ATOM | 5579 | CD1 | LEU | P | 224 | 30.448 | −17.566 | 53.237 | 1.00 | 43.92 | C |
| ATOM | 5580 | CD2 | LEU | P | 224 | 31.080 | −19.480 | 51.730 | 1.00 | 43.04 | C |
| ATOM | 5581 | N | GLU | P | 225 | 31.616 | −14.487 | 49.715 | 1.00 | 43.15 | N |
| ATOM | 5582 | CA | GLU | P | 225 | 32.723 | −13.530 | 49.639 | 1.00 | 43.22 | C |
| ATOM | 5583 | C | GLU | P | 225 | 32.364 | −12.232 | 50.360 | 1.00 | 43.26 | C |
| ATOM | 5584 | O | GLU | P | 225 | 33.172 | −11.677 | 51.103 | 1.00 | 43.22 | O |
| ATOM | 5585 | CB | GLU | P | 225 | 33.096 | −13.239 | 48.183 | 1.00 | 43.08 | C |
| ATOM | 5586 | CG | GLU | P | 225 | 34.326 | −12.355 | 48.024 | 1.00 | 43.12 | C |
| ATOM | 5587 | CD | GLU | P | 225 | 34.593 | −11.946 | 46.585 | 1.00 | 43.30 | C |
| ATOM | 5588 | OE1 | GLU | P | 225 | 34.377 | −12.772 | 45.675 | 1.00 | 43.66 | O |
| ATOM | 5589 | OE2 | GLU | P | 225 | 35.029 | −10.795 | 46.360 | 1.00 | 43.40 | O |
| ATOM | 5590 | N | SER | P | 226 | 31.137 | −11.771 | 50.140 | 1.00 | 43.44 | N |
| ATOM | 5591 | CA | SER | P | 226 | 30.661 | −10.532 | 50.727 | 1.00 | 43.47 | C |
| ATOM | 5592 | C | SER | P | 226 | 30.360 | −10.680 | 52.211 | 1.00 | 43.66 | C |
| ATOM | 5593 | O | SER | P | 226 | 30.610 | −9.757 | 52.988 | 1.00 | 43.95 | O |
| ATOM | 5594 | CB | SER | P | 226 | 29.429 | −10.027 | 49.986 | 1.00 | 43.34 | C |
| ATOM | 5595 | OG | SER | P | 226 | 29.106 | −8.720 | 50.414 | 1.00 | 43.73 | O |
| ATOM | 5596 | N | ALA | P | 227 | 29.828 | −11.840 | 52.600 | 1.00 | 43.69 | N |
| ATOM | 5597 | CA | ALA | P | 227 | 29.513 | −12.123 | 54.004 | 1.00 | 43.55 | C |
| ATOM | 5598 | C | ALA | P | 227 | 30.775 | −12.125 | 54.860 | 1.00 | 43.61 | C |
| ATOM | 5599 | O | ALA | P | 227 | 30.779 | −11.588 | 55.965 | 1.00 | 43.56 | O |
| ATOM | 5600 | CB | ALA | P | 227 | 28.774 | −13.449 | 54.134 | 1.00 | 43.41 | C |
| ATOM | 5601 | N | ARG | P | 228 | 31.846 | −12.709 | 54.325 | 1.00 | 43.76 | N |
| ATOM | 5602 | CA | ARG | P | 228 | 33.119 | −12.833 | 55.038 | 1.00 | 43.94 | C |
| ATOM | 5603 | C | ARG | P | 228 | 33.761 | −11.490 | 55.393 | 1.00 | 44.04 | C |
| ATOM | 5604 | O | ARG | P | 228 | 34.562 | −11.409 | 56.330 | 1.00 | 44.13 | O |
| ATOM | 5605 | CB | ARG | P | 228 | 34.094 | −13.710 | 54.244 | 1.00 | 44.06 | C |
| ATOM | 5606 | CG | ARG | P | 228 | 33.772 | −15.200 | 54.324 | 1.00 | 44.23 | C |
| ATOM | 5607 | CD | ARG | P | 228 | 34.076 | −15.921 | 53.020 | 1.00 | 44.50 | C |
| ATOM | 5608 | NH | ARG | P | 228 | 35.280 | −16.735 | 53.090 | 1.00 | 44.42 | N |
| ATOM | 5609 | CZ | ARG | P | 228 | 35.466 | −17.858 | 52.401 | 1.00 | 44.96 | C |
| ATOM | 5610 | NE1 | ARG | P | 228 | 34.522 | −18.315 | 51.586 | 1.00 | 44.50 | N |
| ATOM | 5611 | NH2 | ARG | P | 228 | 36.599 | −18.536 | 52.534 | 1.00 | 45.62 | N |
| ATOM | 5612 | N | VAL | P | 229 | 33.397 | −10.441 | 54.657 | 1.00 | 44.08 | N |
| ATOM | 5613 | CA | VAL | P | 229 | 33.960 | −9.107 | 54.888 | 1.00 | 44.05 | C |
| ATOM | 5614 | C | VAL | P | 229 | 32.973 | −8.142 | 55.559 | 1.00 | 44.32 | C |
| ATOM | 5615 | O | VAL | P | 229 | 33.381 | −7.304 | 56.371 | 1.00 | 44.27 | O |
| ATOM | 5616 | CB | VAL | P | 229 | 34.615 | −8.507 | 53.593 | 1.00 | 43.87 | C |
| ATOM | 5617 | CG1 | VAL | P | 229 | 33.735 | −8.712 | 52.376 | 1.00 | 43.41 | C |
| ATOM | 5618 | CG2 | VAL | P | 229 | 34.993 | −7.035 | 53.776 | 1.00 | 43.41 | C |
| ATOM | 5619 | N | SER | P | 230 | 31.685 | −8.286 | 55.248 | 1.00 | 44.57 | N |
| ATOM | 5620 | CA | SER | P | 230 | 30.661 | −7.373 | 55.759 | 1.00 | 44.95 | C |
| ATOM | 5621 | C | SER | P | 230 | 30.159 | −7.696 | 57.172 | 1.00 | 45.33 | C |
| ATOM | 5622 | O | SER | P | 230 | 29.748 | −6.796 | 57.908 | 1.00 | 45.39 | O |
| ATOM | 5623 | CB | SER | P | 230 | 29.470 | −7.309 | 54.800 | 1.00 | 44.96 | C |
| ATOM | 5624 | OG | SER | P | 230 | 28.597 | −8.412 | 54.992 | 1.00 | 44.91 | O |
| ATOM | 5625 | N | MET | P | 231 | 30.181 | −8.971 | 57.545 | 1.00 | 45.80 | N |
| ATOM | 5626 | CA | MET | P | 231 | 29.546 | −9.409 | 58.788 | 1.00 | 46.34 | C |
| ATOM | 5627 | C | MET | P | 231 | 30.261 | −8.914 | 60.050 | 1.00 | 46.43 | C |
| ATOM | 5628 | O | MET | P | 231 | 29.677 | −8.902 | 61.136 | 1.00 | 46.15 | O |
| ATOM | 5629 | CB | MET | P | 231 | 29.401 | −10.932 | 58.810 | 1.00 | 46.69 | C |
| ATOM | 5630 | CG | MET | P | 231 | 27.977 | −11.418 | 59.077 | 1.00 | 47.75 | C |
| ATOM | 5631 | SD | MET | P | 231 | 27.401 | −11.097 | 60.763 | 1.00 | 50.94 | S |
| ATOM | 5632 | CE | MET | P | 231 | 25.759 | −11.808 | 60.706 | 1.00 | 48.83 | C |
| ATOM | 5633 | N | VAL | P | 232 | 31.515 | −8.487 | 59.900 | 1.00 | 46.67 | N |
| ATOM | 5634 | CA | VAL | P | 232 | 32.282 | −7.954 | 61.030 | 1.00 | 46.87 | C |
| ATOM | 5635 | C | VAL | P | 232 | 31.816 | −6.550 | 61.402 | 1.00 | 46.91 | C |
| ATOM | 5636 | O | VAL | P | 232 | 32.085 | −6.075 | 62.504 | 1.00 | 47.29 | O |
| ATOM | 5637 | CB | VAL | P | 232 | 33.834 | −7.998 | 60.813 | 1.00 | 46.92 | C |
| ATOM | 5638 | CG1 | VAL | P | 232 | 34.285 | −9.369 | 60.291 | 1.00 | 47.13 | C |
| ATOM | 5639 | CG2 | VAL | P | 232 | 34.303 | −6.895 | 59.887 | 1.00 | 46.96 | C |
| ATOM | 5640 | N | GLY | P | 233 | 31.115 | −5.893 | 60.486 | 1.00 | 46.86 | N |
| ATOM | 5641 | CA | GLY | P | 233 | 30.529 | −4.594 | 60.767 | 1.00 | 47.03 | C |
| ATOM | 5642 | C | GLY | P | 233 | 29.127 | −4.723 | 61.337 | 1.00 | 47.39 | C |
| ATOM | 5643 | O | GLY | P | 233 | 28.495 | −3.722 | 61.688 | 1.00 | 47.45 | O |
| ATOM | 5644 | N | GLN | P | 234 | 28.637 | −5.960 | 61.417 | 1.00 | 47.52 | N |
| ATOM | 5645 | CA | GLN | P | 234 | 27.320 | −6.245 | 61.976 | 1.00 | 47.74 | C |
| ATOM | 5646 | C | GLN | P | 234 | 27.383 | −6.583 | 63.465 | 1.00 | 47.87 | C |
| ATOM | 5647 | O | GLN | P | 234 | 26.413 | −6.389 | 64.197 | 1.00 | 47.60 | O |
| ATOM | 5648 | CB | GLN | P | 234 | 26.667 | −7.398 | 61.223 | 1.00 | 47.75 | C |
| ATOM | 5649 | CG | GLN | P | 234 | 26.001 | −6.996 | 59.926 | 1.00 | 48.68 | C |
| ATOM | 5650 | CD | GLN | P | 234 | 25.086 | −8.081 | 59.384 | 1.00 | 49.86 | C |
| ATOM | 5651 | OE1 | GLN | P | 234 | 24.264 | −8.643 | 60.113 | 1.00 | 50.32 | O |
| ATOM | 5652 | NE2 | GLN | P | 234 | 25.223 | −8.381 | 58.097 | 1.00 | 50.52 | N |
| ATOM | 5653 | N | VAL | P | 235 | 28.530 | −7.092 | 63.902 | 1.00 | 48.26 | N |
| ATOM | 5654 | CA | VAL | P | 235 | 28.692 | −7.565 | 65.271 | 1.00 | 48.76 | C |
| ATOM | 5655 | C | VAL | P | 235 | 29.986 | −7.074 | 65.933 | 1.00 | 49.31 | C |
| ATOM | 5656 | O | VAL | P | 235 | 30.981 | −6.801 | 65.255 | 1.00 | 49.13 | O |
| ATOM | 5657 | CB | VAL | P | 235 | 28.615 | −9.119 | 65.357 | 1.00 | 48.69 | C |
| ATOM | 5658 | CG1 | VAL | P | 235 | 27.187 | −9.602 | 65.174 | 1.00 | 48.18 | C |
| ATOM | 5659 | CG2 | VAL | P | 235 | 29.538 | −9.776 | 64.342 | 1.00 | 48.64 | C |
| ATOM | 5660 | N | LYS | P | 236 | 29.940 | −6.949 | 67.259 | 1.00 | 49.86 | N |
| ATOM | 5661 | CA | LYS | P | 236 | 31.103 | −6.617 | 68.077 | 1.00 | 50.64 | C |
| ATOM | 5662 | C | LYS | P | 236 | 31.314 | −7.746 | 69.090 | 1.00 | 51.05 | C |
| ATOM | 5663 | O | LYS | P | 236 | 30.572 | −8.731 | 69.064 | 1.00 | 51.22 | O |
| ATOM | 5664 | CB | LYS | P | 236 | 30.908 | −5.263 | 68.770 | 1.00 | 50.60 | C |
| ATOM | 5665 | CG | LYS | P | 236 | 29.755 | −5.209 | 69.773 | 1.00 | 50.92 | C |
| ATOM | 5666 | CD | LYS | P | 236 | 29.443 | −3.772 | 70.181 | 1.00 | 51.05 | C |
| ATOM | 5667 | CE | LYS | P | 236 | 28.188 | −3.681 | 71.052 | 1.00 | 51.62 | C |
| ATOM | 5668 | NZ | LYS | P | 236 | 28.425 | −4.142 | 72.453 | 1.00 | 51.26 | N |
| ATOM | 5669 | N | GLN | P | 237 | 32.306 | −7.615 | 69.975 | 1.00 | 51.56 | N |
| ATOM | 5670 | CA | GLN | P | 237 | 32.637 | −8.698 | 70.916 | 1.00 | 52.10 | C |
| ATOM | 5671 | C | GLN | P | 237 | 33.397 | −8.241 | 72.169 | 1.00 | 52.34 | C |
| ATOM | 5672 | O | GLN | P | 237 | 34.054 | −7.200 | 72.154 | 1.00 | 52.34 | O |
| ATOM | 5673 | CB | GLN | P | 237 | 33.399 | −9.816 | 70.179 | 1.00 | 52.20 | C |
| ATOM | 5674 | CG | GLN | P | 237 | 34.898 | −9.920 | 70.450 | 1.00 | 52.38 | C |
| ATOM | 5675 | CD | GLN | P | 237 | 35.217 | −11.002 | 71.472 | 1.00 | 53.04 | C |
| ATOM | 5676 | OE1 | GLN | P | 237 | 34.565 | −12.050 | 71.512 | 1.00 | 53.06 | O |
| ATOM | 5677 | NE2 | GLN | P | 237 | 36.223 | −10.753 | 72.303 | 1.00 | 53.36 | N |
| ATOM | 5678 | N | CYS | P | 238 | 33.297 | −9.025 | 73.245 | 1.00 | 52.75 | N |
| ATOM | 5679 | CA | CYS | P | 238 | 34.047 | −8.772 | 74.487 | 1.00 | 53.19 | C |
| ATOM | 5680 | C | CYS | P | 238 | 34.572 | −10.058 | 75.132 | 1.00 | 53.19 | C |
| ATOM | 5681 | O | CYS | P | 238 | 35.038 | −10.056 | 76.278 | 1.00 | 53.34 | O |
| ATOM | 5682 | CB | CYS | P | 238 | 33.194 | −7.993 | 75.491 | 1.00 | 53.21 | C |
| ATOM | 5683 | SG | CYS | P | 238 | 32.770 | −6.328 | 74.963 | 1.00 | 54.46 | S |
| ATOM | 5684 | N | LYS | P | 283 | 32.938 | −12.547 | 77.203 | 1.00 | 42.00 | N |
| ATOM | 5685 | CA | LYS | P | 283 | 31.695 | −12.297 | 76.475 | 1.00 | 42.15 | C |
| ATOM | 5686 | C | LYS | P | 283 | 31.763 | −12.818 | 75.039 | 1.00 | 42.07 | C |
| ATOM | 5687 | O | LYS | P | 283 | 32.785 | −12.663 | 74.371 | 1.00 | 42.26 | O |
| ATOM | 5688 | CB | LYS | P | 283 | 31.348 | −10.799 | 76.461 | 1.00 | 42.06 | C |
| ATOM | 5689 | CG | LYS | P | 283 | 30.804 | −10.244 | 77.772 | 1.00 | 42.26 | C |
| ATOM | 5690 | CD | LYS | P | 283 | 31.920 | −9.947 | 78.771 | 1.00 | 42.55 | C |
| ATOM | 5691 | CE | LYS | P | 283 | 31.383 | −9.882 | 80.188 | 1.00 | 42.29 | C |
| ATOM | 5692 | NZ | LYS | P | 283 | 32.470 | −9.663 | 81.174 | 1.00 | 41.99 | N |
| ATOM | 5693 | N | PRO | P | 284 | 30.675 | −13.450 | 74.564 | 1.00 | 41.96 | N |
| ATOM | 5694 | CA | PRO | P | 284 | 30.554 | −13.826 | 73.156 | 1.00 | 41.84 | C |
| ATOM | 5695 | C | PRO | P | 284 | 30.329 | −12.610 | 72.250 | 1.00 | 41.69 | C |
| ATOM | 5696 | O | PRO | P | 284 | 30.192 | −11.485 | 72.742 | 1.00 | 41.73 | O |
| ATOM | 5697 | CB | PRO | P | 284 | 29.310 | −14.730 | 73.140 | 1.00 | 41.88 | C |
| ATOM | 5698 | CG | PRO | P | 284 | 29.037 | −15.072 | 74.582 | 1.00 | 42.00 | C |
| ATOM | 5699 | CD | PRO | P | 284 | 29.504 | −13.884 | 75.343 | 1.00 | 41.98 | C |
| ATOM | 5700 | N | TYR | P | 285 | 30.306 | −12.847 | 70.938 | 1.00 | 41.41 | N |
| ATOM | 5701 | CA | TYR | P | 285 | 29.986 | −11.822 | 69.948 | 1.00 | 41.07 | C |
| ATOM | 5702 | C | TYR | P | 285 | 28.507 | −11.496 | 70.013 | 1.00 | 40.79 | C |
| ATOM | 5703 | O | TYR | P | 285 | 27.674 | −12.398 | 70.085 | 1.00 | 40.65 | O |
| ATOM | 5704 | CB | TYR | P | 285 | 30.286 | −12.326 | 68.536 | 1.00 | 41.31 | C |
| ATOM | 5705 | CG | TYR | P | 285 | 31.741 | −12.326 | 68.123 | 1.00 | 41.50 | C |
| ATOM | 5706 | CD1 | TYR | P | 285 | 32.649 | −13.221 | 68.687 | 1.00 | 41.50 | C |
| ATOM | 5707 | CD2 | TYR | P | 285 | 32.201 | −11.452 | 67.136 | 1.00 | 41.64 | C |
| ATOM | 5708 | CE1 | TYR | P | 285 | 33.982 | −13.230 | 68.297 | 1.00 | 41.56 | C |
| ATOM | 5709 | CE2 | TYR | P | 285 | 33.531 | −11.454 | 66.739 | 1.00 | 41.52 | C |
| ATOM | 5710 | CZ | TYR | P | 285 | 34.413 | −12.347 | 67.323 | 1.00 | 41.57 | C |
| ATOM | 5711 | OH | TYR | P | 285 | 35.729 | −12.358 | 66.936 | 1.00 | 41.67 | O |
| ATOM | 5712 | N | THR | P | 286 | 28.183 | −10.207 | 69.971 | 1.00 | 40.48 | N |
| ATOM | 5713 | CA | THR | P | 286 | 26.786 | −9.763 | 69.941 | 1.00 | 40.15 | C |
| ATOM | 5714 | C | THR | P | 286 | 26.545 | −8.685 | 68.872 | 1.00 | 39.77 | C |
| ATOM | 5715 | O | THR | P | 286 | 27.496 | −8.179 | 68.270 | 1.00 | 39.78 | O |
| ATOM | 5716 | CB | THR | P | 286 | 26.299 | −9.295 | 71.339 | 1.00 | 40.23 | C |
| ATOM | 5717 | OG1 | THR | P | 286 | 24.905 | −8.967 | 71.277 | 1.00 | 40.74 | O |
| ATOM | 5718 | CG2 | THR | P | 286 | 27.092 | −8.083 | 71.825 | 1.00 | 40.33 | C |
| ATOM | 5719 | N | TRP | P | 287 | 25.276 | −8.347 | 68.642 | 1.00 | 39.33 | N |
| ATOM | 5720 | CA | TRP | P | 287 | 24.894 | −7.356 | 67.632 | 1.00 | 38.98 | C |
| ATOM | 5721 | C | TRP | P | 287 | 25.393 | −5.956 | 67.955 | 1.00 | 39.12 | C |
| ATOM | 5722 | O | TRP | P | 287 | 25.176 | −5.440 | 69.053 | 1.00 | 39.29 | O |
| ATOM | 5723 | CB | TRP | P | 287 | 23.377 | −7.330 | 67.436 | 1.00 | 38.52 | C |
| ATOM | 5724 | CG | TRP | P | 287 | 22.812 | −8.603 | 66.868 | 1.00 | 38.12 | C |
| ATOM | 5725 | CD1 | TRP | P | 287 | 21.991 | −9.488 | 67.504 | 1.00 | 37.38 | C |
| ATOM | 5726 | CD2 | TRP | P | 287 | 23.033 | −9.133 | 65.551 | 1.00 | 37.42 | C |
| ATOM | 5727 | NE1 | TRP | P | 287 | 21.684 | −10.530 | 66.668 | 1.00 | 37.66 | N |
| ATOM | 5728 | CE2 | TRP | P | 287 | 22.310 | −10.338 | 65.463 | 1.00 | 37.60 | C |
| ATOM | 5729 | CE3 | TRP | P | 287 | 23.776 | −8.707 | 64.440 | 1.00 | 37.02 | C |
| ATOM | 5730 | CZ2 | TRP | P | 287 | 22.302 | −11.124 | 64.304 | 1.00 | 37.51 | C |
| ATOM | 5731 | CZ3 | TRP | P | 287 | 23.768 | −9.487 | 63.291 | 1.00 | 37.21 | C |
| ATOM | 5732 | CH2 | TRP | P | 287 | 23.036 | −10.682 | 63.234 | 1.00 | 37.33 | C |
| ATOM | 5733 | N | ARG | P | 288 | 26.061 | −5.351 | 66.980 | 1.00 | 39.36 | N |
| ATOM | 5734 | CA | ARG | P | 288 | 26.612 | −4.002 | 67.100 | 1.00 | 39.67 | C |
| ATOM | 5735 | C | ARG | P | 288 | 25.530 | −2.998 | 67.494 | 1.00 | 39.68 | C |
| ATOM | 5736 | O | ARG | P | 288 | 25.797 | −2.038 | 68.213 | 1.00 | 39.74 | O |
| ATOM | 5737 | CB | ARG | P | 288 | 27.259 | −3.595 | 65.771 | 1.00 | 39.78 | C |
| ATOM | 5738 | CG | ARG | P | 288 | 28.460 | −2.679 | 65.885 | 1.00 | 40.35 | C |
| ATOM | 5739 | CD | ARG | P | 288 | 29.371 | −2.822 | 64.658 | 1.00 | 41.37 | C |
| ATOM | 5740 | NE | ARG | P | 288 | 30.422 | −3.832 | 64.824 | 1.00 | 41.99 | N |
| ATOM | 5741 | CZ | ARG | P | 288 | 31.600 | −3.610 | 65.413 | 1.00 | 42.47 | C |
| ATOM | 5742 | NH1 | ARG | P | 288 | 31.896 | −2.411 | 65.907 | 1.00 | 41.61 | N |
| ATOM | 5743 | NH2 | ARG | P | 288 | 32.489 | −4.592 | 65.515 | 1.00 | 42.86 | N |
| ATOM | 5744 | N | ILE | P | 289 | 24.308 | −3.243 | 67.026 | 1.00 | 39.80 | N |
| ATOM | 5745 | CA | ILE | P | 289 | 23.163 | −2.396 | 67.339 | 1.00 | 39.84 | C |
| ATOM | 5746 | C | ILE | P | 289 | 21.896 | −3.223 | 67.597 | 1.00 | 40.06 | C |
| ATOM | 5747 | O | ILE | P | 289 | 21.464 | −3.996 | 66.744 | 1.00 | 40.02 | O |
| ATOM | 5748 | CB | ILE | P | 289 | 22.923 | −1.309 | 66.239 | 1.00 | 39.86 | C |
| ATOM | 5749 | CG1 | ILE | P | 289 | 21.720 | −0.425 | 66.601 | 1.00 | 39.51 | C |
| ATOM | 5750 | CG2 | ILE | P | 289 | 22.789 | −1.937 | 64.835 | 1.00 | 39.21 | C |
| ATOM | 5751 | CD1 | ILE | P | 289 | 21.706 | 0.918 | 65.905 | 1.00 | 38.93 | C |
| ATOM | 5752 | N | GLU | P | 290 | 21.323 | −3.064 | 68.788 | 1.00 | 40.35 | N |
| ATOM | 5753 | CA | GLU | P | 290 | 20.031 | −3.662 | 69.117 | 1.00 | 40.73 | C |
| ATOM | 5754 | C | GLU | P | 290 | 18.948 | −2.975 | 68.259 | 1.00 | 40.69 | C |
| ATOM | 5755 | O | GLU | P | 290 | 18.222 | −2.089 | 68.719 | 1.00 | 40.79 | O |
| ATOM | 5756 | CB | GLU | P | 290 | 19.754 | −3.538 | 70.623 | 1.00 | 40.35 | C |
| ATOM | 5757 | CG | GLU | P | 290 | 18.669 | −4.477 | 71.154 | 1.00 | 40.94 | C |
| ATOM | 5758 | CD | GLU | P | 290 | 18.607 | −4.544 | 72.690 | 1.00 | 41.60 | C |
| ATOM | 5759 | OE1 | GLU | P | 290 | 17.591 | −5.043 | 73.223 | 1.00 | 42.85 | O |
| ATOM | 5760 | OE2 | GLU | P | 290 | 19.563 | −4.107 | 73.374 | 1.00 | 42.44 | O |
| ATOM | 5761 | N | LEU | P | 291 | 18.862 | −3.401 | 67.002 | 1.00 | 40.71 | N |
| ATOM | 5762 | CA | LEU | P | 291 | 18.068 | −2.722 | 65.971 | 1.00 | 40.87 | C |
| ATOM | 5763 | C | LEU | P | 291 | 16.564 | −2.715 | 66.231 | 1.00 | 41.12 | C |
| ATOM | 5764 | O | LEU | P | 291 | 15.830 | −1.902 | 65.661 | 1.00 | 41.05 | O |
| ATOM | 5765 | CB | LEU | P | 291 | 18.352 | −3.348 | 64.604 | 1.00 | 40.73 | C |
| ATOM | 5766 | CG | LEU | P | 291 | 18.114 | −2.540 | 63.330 | 1.00 | 40.34 | C |
| ATOM | 5767 | CD1 | LEU | P | 291 | 18.926 | −1.257 | 63.300 | 1.00 | 39.34 | C |
| ATOM | 5768 | CD2 | LEU | P | 291 | 18.460 | −3.416 | 62.145 | 1.00 | 40.80 | C |
| ATOM | 5769 | N | ALA | P | 292 | 16.111 | −3.625 | 67.086 | 1.00 | 41.40 | N |
| ATOM | 5770 | CA | ALA | P | 292 | 14.708 | −3.693 | 67.449 | 1.00 | 41.54 | C |
| ATOM | 5771 | C | ALA | P | 292 | 14.300 | −2.425 | 68.190 | 1.00 | 41.66 | C |
| ATOM | 5772 | O | ALA | P | 292 | 13.135 | −2.042 | 68.159 | 1.00 | 41.91 | O |
| ATOM | 5773 | CB | ALA | P | 292 | 14.437 | −4.925 | 68.285 | 1.00 | 41.59 | C |
| ATOM | 5774 | N | LYS | P | 293 | 15.272 | −1.773 | 68.829 | 1.00 | 41.76 | N |
| ATOM | 5775 | CA | LYS | P | 293 | 15.045 | −0.529 | 69.571 | 1.00 | 42.12 | C |
| ATOM | 5776 | C | LYS | P | 293 | 14.629 | 0.616 | 68.661 | 1.00 | 41.97 | C |
| ATOM | 5777 | O | LYS | P | 293 | 13.921 | 1.525 | 69.085 | 1.00 | 41.91 | O |
| ATOM | 5778 | CB | LYS | P | 293 | 16.312 | −0.099 | 70.325 | 1.00 | 42.24 | C |
| ATOM | 5779 | CG | LYS | P | 293 | 16.700 | −0.941 | 71.539 | 1.00 | 42.49 | C |
| ATOM | 5780 | CD | LYS | P | 293 | 17.821 | −0.241 | 72.313 | 1.00 | 42.65 | C |
| ATOM | 5781 | CE | LYS | P | 293 | 18.285 | −1.036 | 73.533 | 1.00 | 43.40 | C |
| ATOM | 5782 | NZ | LYS | P | 293 | 17.297 | −1.021 | 74.649 | 1.00 | 43.46 | N |
| ATOM | 5783 | N | THR | P | 294 | 15.085 | 0.563 | 67.411 | 1.00 | 42.10 | N |
| ATOM | 5784 | CA | THR | P | 294 | 14.923 | 1.661 | 66.461 | 1.00 | 41.93 | C |
| ATOM | 5785 | C | THR | P | 294 | 13.544 | 1.684 | 65.807 | 1.00 | 42.29 | C |
| ATOM | 5786 | O | THR | P | 294 | 13.283 | 2.514 | 64.936 | 1.00 | 42.25 | O |
| ATOM | 5787 | CB | THR | P | 294 | 15.986 | 1.601 | 65.352 | 1.00 | 41.80 | C |
| ATOM | 5788 | OG1 | THR | P | 294 | 15.727 | 0.474 | 64.507 | 1.00 | 41.73 | O |
| ATOM | 5789 | CG2 | THR | P | 294 | 17.389 | 1.497 | 65.939 | 1.00 | 40.91 | C |
| ATOM | 5790 | N | GLU | P | 295 | 12.662 | 0.785 | 66.237 | 1.00 | 42.75 | N |
| ATOM | 5791 | CA | GLU | P | 295 | 11.324 | 0.674 | 65.659 | 1.00 | 43.25 | C |
| ATOM | 5792 | C | GLU | P | 295 | 10.545 | 1.995 | 65.636 | 1.00 | 43.15 | C |
| ATOM | 5793 | O | GLU | P | 295 | 9.792 | 2.256 | 64.694 | 1.00 | 43.12 | O |
| ATOM | 5794 | CB | GLU | P | 295 | 10.510 | −0.398 | 66.376 | 1.00 | 43.48 | C |
| ATOM | 5795 | CG | GLU | P | 295 | 9.493 | −1.070 | 65.459 | 1.00 | 44.89 | C |
| ATOM | 5796 | CD | GLU | P | 295 | 8.190 | −1.434 | 66.152 | 1.00 | 46.46 | C |
| ATOM | 5797 | OE1 | GLU | P | 295 | 8.222 | −1.875 | 67.332 | 1.00 | 46.98 | O |
| ATOM | 5798 | OE2 | GLU | P | 295 | 7.132 | −1.285 | 65.496 | 1.00 | 46.40 | O |
| ATOM | 5799 | N | LYS | P | 296 | 10.743 | 2.823 | 66.661 | 1.00 | 43.06 | N |
| ATOM | 5800 | CA | LYS | P | 296 | 10.063 | 4.117 | 66.768 | 1.00 | 43.05 | C |
| ATOM | 5801 | C | LYS | P | 296 | 10.403 | 5.103 | 65.642 | 1.00 | 43.10 | C |
| ATOM | 5802 | O | LYS | P | 296 | 9.634 | 6.030 | 65.378 | 1.00 | 43.24 | O |
| ATOM | 5803 | CB | LYS | P | 296 | 10.318 | 4.760 | 68.136 | 1.00 | 43.05 | C |
| ATOM | 5804 | CG | LYS | P | 296 | 11.782 | 5.088 | 68.440 | 1.00 | 43.13 | C |
| ATOM | 5805 | CD | LYS | P | 296 | 11.909 | 6.149 | 69.539 | 1.00 | 43.19 | C |
| ATOM | 5806 | CE | LYS | P | 296 | 11.792 | 5.564 | 70.945 | 1.00 | 43.54 | C |
| ATOM | 5807 | NZ | LYS | P | 296 | 13.056 | 4.905 | 71.387 | 1.00 | 43.67 | N |
| ATOM | 5808 | N | TYR | P | 297 | 11.540 | 4.901 | 64.978 | 1.00 | 43.01 | N |
| ATOM | 5809 | CA | TYR | P | 297 | 11.968 | 5.793 | 63.896 | 1.00 | 42.75 | C |
| ATOM | 5810 | C | TYR | P | 297 | 11.622 | 5.284 | 62.496 | 1.00 | 42.40 | C |
| ATOM | 5811 | O | TYR | P | 297 | 11.880 | 5.977 | 61.511 | 1.00 | 42.31 | O |
| ATOM | 5812 | CB | TYR | P | 297 | 13.472 | 6.091 | 63.996 | 1.00 | 42.93 | C |
| ATOM | 5813 | CG | TYR | P | 297 | 13.883 | 6.674 | 65.328 | 1.00 | 43.34 | C |
| ATOM | 5814 | CD1 | TYR | P | 297 | 13.513 | 7.971 | 65.686 | 1.00 | 43.53 | C |
| ATOM | 5815 | CD2 | TYR | P | 297 | 14.632 | 5.927 | 66.237 | 1.00 | 43.60 | C |
| ATOM | 5816 | CE1 | TYR | P | 297 | 13.876 | 8.511 | 66.915 | 1.00 | 43.58 | C |
| ATOM | 5817 | CE2 | TYR | P | 297 | 15.003 | 6.457 | 67.470 | 1.00 | 43.56 | C |
| ATOM | 5818 | CZ | TYR | P | 297 | 14.619 | 7.751 | 67.801 | 1.00 | 43.68 | C |
| ATOM | 5819 | OH | TYR | P | 297 | 14.973 | 8.290 | 69.020 | 1.00 | 43.72 | O |
| ATOM | 5820 | N | TRP | P | 298 | 11.028 | 4.092 | 62.410 | 1.00 | 42.02 | N |
| ATOM | 5821 | CA | TRP | P | 298 | 10.776 | 3.451 | 61.110 | 1.00 | 41.78 | C |
| ATOM | 5822 | C | TRP | P | 298 | 9.707 | 4.133 | 60.268 | 1.00 | 41.71 | C |
| ATOM | 5823 | O | TRP | P | 298 | 9.818 | 4.167 | 59.037 | 1.00 | 41.53 | O |
| ATOM | 5824 | CB | TRP | P | 298 | 10.426 | 1.972 | 61.254 | 1.00 | 41.56 | C |
| ATOM | 5825 | CG | TRP | P | 298 | 11.512 | 1.128 | 61.825 | 1.00 | 41.32 | C |
| ATOM | 5826 | CD1 | TRP | P | 298 | 12.784 | 1.515 | 62.148 | 1.00 | 40.92 | C |
| ATOM | 5827 | CD2 | TRP | P | 298 | 11.432 | −0.268 | 62.115 | 1.00 | 40.94 | C |
| ATOM | 5828 | NE1 | TRP | P | 298 | 13.490 | 0.449 | 62.643 | 1.00 | 40.93 | N |
| ATOM | 5829 | CE2 | TRP | P | 298 | 12.684 | −0.660 | 62.631 | 1.00 | 40.90 | C |
| ATOM | 5830 | CE3 | TRP | P | 298 | 10.417 | −1.227 | 61.995 | 1.00 | 40.63 | C |
| ATOM | 5831 | CZ2 | TRP | P | 298 | 12.948 | −1.971 | 63.031 | 1.00 | 41.30 | C |
| ATOM | 5832 | CZ3 | TRP | P | 298 | 10.681 | −2.529 | 62.390 | 1.00 | 41.00 | C |
| ATOM | 5833 | CH2 | TRP | P | 298 | 11.937 | −2.890 | 62.901 | 1.00 | 41.18 | C |
| ATOM | 5834 | N | ASP | P | 299 | 8.671 | 4.649 | 60.927 | 1.00 | 41.66 | N |
| ATOM | 5835 | CA | ASP | P | 299 | 7.628 | 5.407 | 60.239 | 1.00 | 41.75 | C |
| ATOM | 5836 | C | ASP | P | 299 | 8.195 | 6.662 | 59.576 | 1.00 | 41.72 | C |
| ATOM | 5837 | O | ASP | P | 299 | 7.889 | 6.949 | 58.414 | 1.00 | 42.02 | O |
| ATOM | 5838 | CB | ASP | P | 299 | 6.479 | 5.760 | 61.181 | 1.00 | 41.84 | C |
| ATOM | 5839 | CG | ASP | P | 299 | 5.299 | 6.358 | 60.448 | 1.00 | 42.08 | C |
| ATOM | 5840 | OD1 | ASP | P | 299 | 4.370 | 5.599 | 60.098 | 1.00 | 42.38 | O |
| ATOM | 5841 | OD2 | ASP | P | 299 | 5.312 | 7.580 | 60.197 | 1.00 | 42.52 | O |
| ATOM | 5842 | N | GLY | P | 300 | 9.039 | 7.380 | 60.311 | 1.00 | 41.55 | N |
| ATOM | 5843 | CA | GLY | P | 300 | 9.721 | 8.560 | 59.799 | 1.00 | 41.55 | C |
| ATOM | 5844 | C | GLY | P | 300 | 10.692 | 8.280 | 58.665 | 1.00 | 41.64 | C |
| ATOM | 5845 | O | GLY | P | 300 | 10.995 | 9.175 | 57.873 | 1.00 | 41.86 | O |
| ATOM | 5846 | N | TRP | P | 301 | 11.178 | 7.043 | 58.576 | 1.00 | 41.65 | N |
| ATOM | 5847 | CA | TRP | P | 301 | 12.111 | 6.663 | 57.510 | 1.00 | 41.55 | C |
| ATOM | 5848 | C | TRP | P | 301 | 11.424 | 6.501 | 56.162 | 1.00 | 41.49 | C |
| ATOM | 5849 | O | TRP | P | 301 | 11.834 | 7.124 | 55.181 | 1.00 | 41.83 | O |
| ATOM | 5850 | CB | TRP | P | 301 | 12.869 | 5.372 | 57.843 | 1.00 | 41.64 | C |
| ATOM | 5851 | CG | TRP | P | 301 | 13.851 | 5.460 | 58.979 | 1.00 | 41.69 | C |
| ATOM | 5852 | CD1 | TRP | P | 301 | 14.266 | 6.586 | 59.633 | 1.00 | 41.82 | C |
| ATOM | 5853 | CD2 | TRP | P | 301 | 14.572 | 4.371 | 59.563 | 1.00 | 41.68 | C |
| ATOM | 5854 | NE1 | TRP | P | 301 | 15.178 | 6.261 | 60.604 | 1.00 | 41.57 | N |
| ATOM | 5855 | CE2 | TRP | P | 301 | 15.388 | 4.908 | 60.583 | 1.00 | 41.55 | C |
| ATOM | 5856 | CE3 | TRP | P | 301 | 14.603 | 2.989 | 59.328 | 1.00 | 42.04 | C |
| ATOM | 5857 | CZ2 | TRP | P | 301 | 16.230 | 4.113 | 61.372 | 1.00 | 41.81 | C |
| ATOM | 5858 | CZ3 | TRP | P | 301 | 15.437 | 2.197 | 60.114 | 1.00 | 42.29 | C |
| ATOM | 5859 | CH2 | TRP | P | 301 | 16.239 | 2.766 | 61.129 | 1.00 | 41.99 | C |
| ATOM | 5860 | N | PHE | P | 302 | 10.384 | 5.666 | 56.115 | 1.00 | 41.25 | N |
| ATOM | 5861 | CA | PHE | P | 302 | 9.757 | 5.282 | 54.842 | 1.00 | 41.03 | C |
| ATOM | 5862 | C | PHE | P | 302 | 8.535 | 6.119 | 54.438 | 1.00 | 40.90 | C |
| ATOM | 5863 | O | PHE | P | 302 | 8.163 | 6.120 | 53.266 | 1.00 | 40.76 | O |
| ATOM | 5864 | CB | PHE | P | 302 | 9.380 | 3.791 | 54.820 | 1.00 | 41.03 | C |
| ATOM | 5865 | CG | PHE | P | 302 | 10.443 | 2.886 | 55.356 | 1.00 | 41.00 | C |
| ATOM | 5866 | CD1 | PHE | P | 302 | 11.553 | 2.557 | 54.587 | 1.00 | 41.37 | C |
| ATOM | 5867 | CD2 | PHE | P | 302 | 10.331 | 2.355 | 56.635 | 1.00 | 41.08 | C |
| ATOM | 5868 | CE1 | PHE | P | 302 | 12.542 | 1.721 | 55.094 | 1.00 | 41.58 | C |
| ATOM | 5869 | CE2 | PHE | P | 302 | 11.312 | 1.517 | 57.149 | 1.00 | 41.42 | C |
| ATOM | 5870 | CZ | PHE | P | 302 | 12.416 | 1.199 | 56.379 | 1.00 | 41.44 | C |
| ATOM | 5871 | N | ARG | P | 303 | 7.900 | 6.806 | 55.388 | 1.00 | 40.65 | N |
| ATOM | 5872 | CA | ARG | P | 303 | 6.704 | 7.578 | 55.048 | 1.00 | 40.46 | C |
| ATOM | 5873 | C | ARG | P | 303 | 7.017 | 8.595 | 53.957 | 1.00 | 40.36 | C |
| ATOM | 5874 | O | ARG | P | 303 | 8.064 | 9.232 | 53.980 | 1.00 | 40.47 | O |
| ATOM | 5875 | CB | ARG | P | 303 | 6.104 | 8.262 | 56.272 | 1.00 | 40.32 | C |
| ATOM | 5876 | CG | ARG | P | 303 | 4.683 | 8.730 | 56.048 | 1.00 | 40.06 | C |
| ATOM | 5877 | CD | ARG | P | 303 | 3.987 | 9.100 | 57.356 | 1.00 | 39.67 | C |
| ATOM | 5878 | NE | ARG | P | 303 | 3.348 | 7.959 | 58.009 | 1.00 | 39.11 | N |
| ATOM | 5879 | CZ | ARG | P | 303 | 2.235 | 7.366 | 57.580 | 1.00 | 38.99 | C |
| ATOM | 5880 | NH1 | ARG | P | 303 | 1.629 | 7.781 | 56.473 | 1.00 | 38.86 | N |
| ATOM | 5881 | NH2 | ARG | P | 303 | 1.727 | 6.347 | 58.257 | 1.00 | 38.53 | N |
| ATOM | 5882 | N | GLY | P | 304 | 6.119 | 8.702 | 52.982 | 1.00 | 40.31 | N |
| ATOM | 5883 | CA | GLY | P | 304 | 6.257 | 9.627 | 51.858 | 1.00 | 39.92 | C |
| ATOM | 5884 | C | GLY | P | 304 | 7.531 | 9.509 | 51.050 | 1.00 | 39.83 | C |
| ATOM | 5885 | O | GLY | P | 304 | 7.852 | 10.410 | 50.278 | 1.00 | 40.26 | O |
| ATOM | 5886 | N | LEU | P | 305 | 8.254 | 8.405 | 51.221 | 1.00 | 39.59 | N |
| ATOM | 5887 | CA | LEU | P | 305 | 9.552 | 8.198 | 50.574 | 1.00 | 39.54 | C |
| ATOM | 5888 | C | LEU | P | 305 | 9.454 | 8.098 | 49.044 | 1.00 | 39.69 | C |
| ATOM | 5889 | O | LEU | P | 305 | 10.368 | 8.511 | 48.326 | 1.00 | 39.54 | O |
| ATOM | 5890 | CB | LEU | P | 305 | 10.246 | 6.953 | 51.158 | 1.00 | 39.61 | C |
| ATOM | 5891 | CG | LEU | P | 305 | 11.635 | 6.506 | 50.676 | 1.00 | 39.61 | C |
| ATOM | 5892 | CD1 | LEU | P | 305 | 12.728 | 7.475 | 51.130 | 1.00 | 39.16 | C |
| ATOM | 5893 | CD2 | LEU | P | 305 | 11.937 | 5.090 | 51.145 | 1.00 | 39.51 | C |
| ATOM | 5894 | N | SER | P | 306 | 8.354 | 7.545 | 48.547 | 1.00 | 39.84 | N |
| ATOM | 5895 | CA | SER | P | 306 | 8.166 | 7.443 | 47.106 | 1.00 | 40.09 | C |
| ATOM | 5896 | C | SER | P | 306 | 8.002 | 8.830 | 46.485 | 1.00 | 40.30 | C |
| ATOM | 5897 | O | SER | P | 306 | 8.767 | 9.199 | 45.590 | 1.00 | 40.36 | O |
| ATOM | 5898 | CB | SER | P | 306 | 7.001 | 6.516 | 46.766 | 1.00 | 39.95 | C |
| ATOM | 5899 | OG | SER | P | 306 | 7.345 | 5.178 | 47.088 | 1.00 | 39.91 | O |
| ATOM | 5900 | N | ASN | P | 307 | 7.035 | 9.600 | 46.985 | 1.00 | 40.35 | N |
| ATOM | 5901 | CA | ASN | P | 307 | 6.834 | 10.976 | 46.540 | 1.00 | 40.44 | C |
| ATOM | 5902 | C | ASN | P | 307 | 8.067 | 11.847 | 46.742 | 1.00 | 40.63 | C |
| ATOM | 5903 | O | ASN | P | 307 | 8.360 | 12.720 | 45.924 | 1.00 | 40.82 | O |
| ATOM | 5904 | CB | ASN | P | 307 | 5.631 | 11.606 | 47.246 | 1.00 | 40.40 | C |
| ATOM | 5905 | CG | ASN | P | 307 | 4.315 | 11.308 | 46.543 | 1.00 | 40.24 | C |
| ATOM | 5906 | OD1 | ASN | P | 307 | 3.264 | 11.816 | 46.941 | 1.00 | 39.42 | O |
| ATOM | 5907 | ND2 | ASN | P | 307 | 4.367 | 10.488 | 45.488 | 1.00 | 39.66 | N |
| ATOM | 5908 | N | LEU | P | 308 | 8.785 | 11.604 | 47.835 | 1.00 | 40.79 | N |
| ATOM | 5909 | CA | LEU | P | 308 | 10.024 | 12.316 | 48.102 | 1.00 | 41.03 | C |
| ATOM | 5910 | C | LEU | P | 308 | 11.107 | 11.976 | 47.064 | 1.00 | 41.24 | C |
| ATOM | 5911 | O | LEU | P | 308 | 11.722 | 12.875 | 46.485 | 1.00 | 41.18 | O |
| ATOM | 5912 | CB | LEU | P | 308 | 10.510 | 12.042 | 49.529 | 1.00 | 40.78 | C |
| ATOM | 5913 | CG | LEU | P | 308 | 11.468 | 13.089 | 50.113 | 1.00 | 40.85 | C |
| ATOM | 5914 | CD1 | LEU | P | 308 | 10.896 | 14.511 | 50.033 | 1.00 | 40.21 | C |
| ATOM | 5915 | CD2 | LEU | P | 308 | 11.837 | 12.737 | 51.546 | 1.00 | 40.97 | C |
| ATOM | 5916 | N | PHE | P | 309 | 11.317 | 10.682 | 46.835 | 1.00 | 41.39 | N |
| ATOM | 5917 | CA | PHE | P | 309 | 12.267 | 10.192 | 45.843 | 1.00 | 41.92 | C |
| ATOM | 5918 | C | PHE | P | 309 | 11.975 | 10.800 | 44.470 | 1.00 | 42.21 | C |
| ATOM | 5919 | O | PHE | P | 309 | 12.880 | 11.280 | 43.787 | 1.00 | 42.23 | O |
| ATOM | 5920 | CB | PHE | P | 309 | 12.207 | 8.659 | 45.811 | 1.00 | 42.04 | C |
| ATOM | 5921 | CG | PHE | P | 309 | 12.936 | 8.020 | 44.655 | 1.00 | 42.64 | C |
| ATOM | 5922 | CD1 | PHE | P | 309 | 14.315 | 7.834 | 44.694 | 1.00 | 43.60 | C |
| ATOM | 5923 | CD2 | PHE | P | 309 | 12.231 | 7.551 | 43.546 | 1.00 | 42.98 | C |
| ATOM | 5924 | CE1 | PHE | P | 309 | 14.985 | 7.217 | 43.628 | 1.00 | 43.61 | C |
| ATOM | 5925 | CE2 | PHE | P | 309 | 12.892 | 6.934 | 42.479 | 1.00 | 42.35 | C |
| ATOM | 5926 | CZ | PHE | P | 309 | 14.264 | 6.766 | 42.520 | 1.00 | 42.72 | C |
| ATOM | 5927 | N | LEU | P | 310 | 10.695 | 10.804 | 44.100 | 1.00 | 42.47 | N |
| ATOM | 5928 | CA | LEU | P | 310 | 10.229 | 11.341 | 42.828 | 1.00 | 42.34 | C |
| ATOM | 5929 | C | LEU | P | 310 | 10.431 | 12.842 | 42.679 | 1.00 | 42.56 | C |
| ATOM | 5930 | O | LEU | P | 310 | 10.502 | 13.341 | 41.556 | 1.00 | 42.88 | O |
| ATOM | 5931 | CB | LEU | P | 310 | 8.749 | 11.011 | 42.624 | 1.00 | 42.25 | C |
| ATOM | 5932 | CG | LEU | P | 310 | 8.376 | 9.568 | 42.268 | 1.00 | 41.78 | C |
| ATOM | 5933 | CD1 | LEU | P | 310 | 6.885 | 9.372 | 42.374 | 1.00 | 40.54 | C |
| ATOM | 5934 | CD2 | LEU | P | 310 | 8.857 | 9.188 | 40.872 | 1.00 | 42.24 | C |
| ATOM | 5935 | N | SER | P | 311 | 10.525 | 13.560 | 43.796 | 1.00 | 42.70 | N |
| ATOM | 5936 | CA | SER | P | 311 | 10.625 | 15.024 | 43.756 | 1.00 | 42.89 | C |
| ATOM | 5937 | C | SER | P | 311 | 12.050 | 15.560 | 43.621 | 1.00 | 43.19 | C |
| ATOM | 5938 | O | SER | P | 311 | 12.236 | 16.761 | 43.433 | 1.00 | 43.17 | O |
| ATOM | 5939 | CB | SER | P | 311 | 9.937 | 15.656 | 44.972 | 1.00 | 42.85 | C |
| ATOM | 5940 | OG | SER | P | 311 | 10.755 | 15.601 | 46.125 | 1.00 | 42.81 | O |
| ATOM | 5941 | N | CYS | P | 312 | 13.047 | 14.681 | 43.720 | 1.00 | 43.66 | N |
| ATOM | 5942 | CA | CYS | P | 312 | 14.447 | 15.089 | 43.579 | 1.00 | 44.20 | C |
| ATOM | 5943 | C | CYS | P | 312 | 14.785 | 15.231 | 42.104 | 1.00 | 44.45 | C |
| ATOM | 5944 | O | CYS | P | 312 | 14.900 | 14.233 | 41.402 | 1.00 | 44.72 | O |
| ATOM | 5945 | CB | CYS | P | 312 | 15.387 | 14.082 | 44.233 | 1.00 | 44.13 | C |
| ATOM | 5946 | SG | CYS | P | 312 | 14.970 | 13.690 | 45.941 | 1.00 | 45.23 | S |
| ATOM | 5947 | N | PRO | P | 313 | 14.971 | 16.476 | 41.633 | 1.00 | 44.79 | N |
| ATOM | 5948 | CA | PRO | P | 313 | 15.036 | 16.740 | 40.190 | 1.00 | 44.92 | C |
| ATOM | 5949 | C | PRO | P | 313 | 16.401 | 16.394 | 39.585 | 1.00 | 44.86 | C |
| ATOM | 5950 | O | PRO | P | 313 | 16.982 | 17.193 | 38.841 | 1.00 | 45.12 | O |
| ATOM | 5951 | CB | PRO | P | 313 | 14.746 | 18.252 | 40.082 | 1.00 | 44.96 | C |
| ATOM | 5952 | CG | PRO | P | 313 | 14.691 | 18.787 | 41.518 | 1.00 | 45.03 | C |
| ATOM | 5953 | CD | PRO | P | 313 | 15.182 | 17.699 | 42.428 | 1.00 | 44.88 | C |
| ATOM | 5954 | N | ILE | P | 314 | 16.899 | 15.207 | 39.909 | 1.00 | 44.50 | N |
| ATOM | 5955 | CA | ILE | P | 314 | 18.199 | 14.753 | 39.434 | 1.00 | 44.16 | C |
| ATOM | 5956 | C | ILE | P | 314 | 18.055 | 13.326 | 38.921 | 1.00 | 43.95 | C |
| ATOM | 5957 | O | ILE | P | 314 | 17.133 | 12.617 | 39.338 | 1.00 | 43.95 | O |
| ATOM | 5958 | CB | ILE | P | 314 | 19.289 | 14.823 | 40.547 | 1.00 | 44.28 | C |
| ATOM | 5959 | CG1 | ILE | P | 314 | 18.876 | 14.016 | 41.782 | 1.00 | 43.82 | C |
| ATOM | 5960 | CG2 | ILE | P | 314 | 19.600 | 16.276 | 40.925 | 1.00 | 44.11 | C |
| ATOM | 5961 | CD1 | ILE | P | 314 | 20.045 | 13.561 | 42.598 | 1.00 | 43.50 | C |
| ATOM | 5962 | N | PRO | P | 315 | 18.952 | 12.898 | 38.009 | 1.00 | 43.68 | N |
| ATOM | 5963 | CA | PRO | P | 315 | 18.845 | 11.529 | 37.490 | 1.00 | 43.40 | C |
| ATOM | 5964 | C | PRO | P | 315 | 18.802 | 10.507 | 38.624 | 1.00 | 42.88 | C |
| ATOM | 5965 | O | PRO | P | 315 | 19.666 | 10.530 | 39.506 | 1.00 | 43.04 | O |
| ATOM | 5966 | CB | PRO | P | 315 | 20.120 | 11.370 | 36.653 | 1.00 | 43.40 | C |
| ATOM | 5967 | CG | PRO | P | 315 | 20.464 | 12.762 | 36.238 | 1.00 | 43.55 | C |
| ATOM | 5968 | CD | PRO | P | 315 | 20.085 | 13.625 | 37.406 | 1.00 | 43.65 | C |
| ATOM | 5969 | N | LYS | P | 316 | 17.785 | 9.650 | 38.608 | 1.00 | 42.23 | N |
| ATOM | 5970 | CA | LYS | P | 316 | 17.590 | 8.667 | 39.671 | 1.00 | 41.69 | C |
| ATOM | 5971 | C | LYS | P | 316 | 17.648 | 7.229 | 39.173 | 1.00 | 41.71 | C |
| ATOM | 5972 | O | LYS | P | 316 | 17.172 | 6.912 | 38.075 | 1.00 | 41.84 | O |
| ATOM | 5973 | CB | LYS | P | 316 | 16.269 | 8.904 | 40.408 | 1.00 | 41.64 | C |
| ATOM | 5974 | CG | LYS | P | 316 | 16.212 | 10.194 | 41.209 | 1.00 | 40.84 | C |
| ATOM | 5975 | CD | LYS | P | 316 | 14.836 | 10.405 | 41.828 | 1.00 | 40.90 | C |
| ATOM | 5976 | CE | LYS | P | 316 | 13.740 | 10.733 | 40.798 | 1.00 | 39.19 | C |
| ATOM | 5977 | NZ | LYS | P | 316 | 13.937 | 12.027 | 40.093 | 1.00 | 37.28 | N |
| ATOM | 5978 | N | LEU | P | 317 | 18.238 | 6.371 | 40.003 | 1.00 | 41.31 | N |
| ATOM | 5979 | CA | LEU | P | 317 | 18.282 | 4.936 | 39.769 | 1.00 | 40.89 | C |
| ATOM | 5980 | C | LEU | P | 317 | 17.635 | 4.192 | 40.945 | 1.00 | 40.70 | C |
| ATOM | 5981 | O | LEU | P | 317 | 17.835 | 4.554 | 42.105 | 1.00 | 40.52 | O |
| ATOM | 5982 | CB | LEU | P | 317 | 19.727 | 4.480 | 39.555 | 1.00 | 40.79 | C |
| ATOM | 5983 | CG | LEU | P | 317 | 20.000 | 3.018 | 39.177 | 1.00 | 40.99 | C |
| ATOM | 5984 | CD1 | LEU | P | 317 | 19.251 | 2.577 | 37.909 | 1.00 | 39.73 | C |
| ATOM | 5985 | CD2 | LEU | P | 317 | 21.504 | 2.794 | 39.027 | 1.00 | 41.01 | C |
| ATOM | 5986 | N | LEU | P | 318 | 16.837 | 3.174 | 40.627 | 1.00 | 40.47 | N |
| ATOM | 5987 | CA | LEU | P | 318 | 16.216 | 2.323 | 41.629 | 1.00 | 40.28 | C |
| ATOM | 5988 | C | LEU | P | 318 | 16.450 | 0.851 | 41.287 | 1.00 | 40.35 | C |
| ATOM | 5989 | O | LEU | P | 318 | 16.043 | 0.380 | 40.227 | 1.00 | 40.40 | O |
| ATOM | 5990 | CB | LEU | P | 318 | 14.721 | 2.627 | 41.747 | 1.00 | 40.16 | C |
| ATOM | 5991 | CG | LEU | P | 318 | 13.921 | 1.818 | 42.777 | 1.00 | 39.81 | C |
| ATOM | 5992 | CD1 | LEU | P | 318 | 14.032 | 2.411 | 44.165 | 1.00 | 39.14 | C |
| ATOM | 5993 | CD2 | LEU | P | 318 | 12.467 | 1.718 | 42.372 | 1.00 | 39.93 | C |
| ATOM | 5994 | N | LEU | P | 319 | 17.126 | 0.138 | 42.185 | 1.00 | 40.48 | N |
| ATOM | 5995 | CA | LEU | P | 319 | 17.423 | −1.281 | 41.988 | 1.00 | 40.55 | C |
| ATOM | 5996 | C | LEU | P | 319 | 16.619 | −2.141 | 42.960 | 1.00 | 40.87 | C |
| ATOM | 5997 | O | LEU | P | 319 | 16.629 | −1.901 | 44.172 | 1.00 | 40.93 | O |
| ATOM | 5998 | CB | LEU | P | 319 | 18.925 | −1.551 | 42.133 | 1.00 | 40.25 | C |
| ATOM | 5999 | CG | LEU | P | 319 | 19.910 | −0.749 | 41.266 | 1.00 | 40.15 | C |
| ATOM | 6000 | CD1 | LEU | P | 319 | 21.350 | −1.129 | 41.586 | 1.00 | 39.87 | C |
| ATOM | 6001 | CD2 | LEU | P | 319 | 19.658 | −0.911 | 39.779 | 1.00 | 38.93 | C |
| ATOM | 6002 | N | LEU | P | 320 | 15.911 | −3.131 | 42.416 | 1.00 | 41.16 | N |
| ATOM | 6003 | CA | LEU | P | 320 | 15.060 | −4.017 | 43.212 | 1.00 | 41.63 | C |
| ATOM | 6004 | C | LEU | P | 320 | 15.376 | −5.493 | 42.970 | 1.00 | 42.00 | C |
| ATOM | 6005 | O | LEU | P | 320 | 15.830 | −5.877 | 41.881 | 1.00 | 42.12 | O |
| ATOM | 6006 | CB | LEU | P | 320 | 13.576 | −3.772 | 42.905 | 1.00 | 41.46 | C |
| ATOM | 6007 | CG | LEU | P | 320 | 13.004 | −2.351 | 42.890 | 1.00 | 41.66 | C |
| ATOM | 6008 | CD1 | LEU | P | 320 | 11.572 | −2.374 | 42.322 | 1.00 | 41.28 | C |
| ATOM | 6009 | CD2 | LEU | P | 320 | 13.064 | −1.657 | 44.267 | 1.00 | 40.43 | C |
| ATOM | 6010 | N | ALA | P | 321 | 15.119 | −6.319 | 43.984 | 1.00 | 42.21 | N |
| ATOM | 6011 | CA | ALA | P | 321 | 15.198 | −7.769 | 43.825 | 1.00 | 42.42 | C |
| ATOM | 6012 | C | ALA | P | 321 | 13.976 | −8.320 | 43.084 | 1.00 | 42.50 | C |
| ATOM | 6013 | O | ALA | P | 321 | 13.969 | −9.478 | 42.685 | 1.00 | 42.86 | O |
| ATOM | 6014 | CB | ALA | P | 321 | 15.365 | −8.456 | 45.177 | 1.00 | 42.33 | C |
| ATOM | 6015 | N | GLY | P | 322 | 12.947 | −7.492 | 42.904 | 1.00 | 42.54 | N |
| ATOM | 6016 | CA | GLY | P | 322 | 11.731 | −7.905 | 42.197 | 1.00 | 42.55 | C |
| ATOM | 6017 | C | GLY | P | 322 | 10.697 | −6.805 | 42.021 | 1.00 | 42.46 | C |
| ATOM | 6018 | O | GLY | P | 322 | 10.623 | −5.884 | 42.831 | 1.00 | 42.51 | O |
| ATOM | 6019 | N | VAL | P | 323 | 9.891 | −6.902 | 40.964 | 1.00 | 42.34 | N |
| ATOM | 6020 | CA | VAL | P | 323 | 8.837 | −5.921 | 40.708 | 1.00 | 42.27 | C |
| ATOM | 6021 | C | VAL | P | 323 | 7.807 | −5.865 | 41.833 | 1.00 | 42.44 | C |
| ATOM | 6022 | O | VAL | P | 323 | 7.078 | −4.892 | 41.951 | 1.00 | 42.60 | O |
| ATOM | 6023 | CB | VAL | P | 323 | 8.105 | −6.161 | 39.366 | 1.00 | 42.14 | C |
| ATOM | 6024 | CG1 | VAL | P | 323 | 9.050 | −5.984 | 38.202 | 1.00 | 41.86 | C |
| ATOM | 6025 | CG2 | VAL | P | 323 | 7.434 | −7.531 | 39.338 | 1.00 | 42.14 | C |
| ATOM | 6026 | N | ASP | P | 324 | 7.761 | −6.912 | 42.653 | 1.00 | 42.74 | N |
| ATOM | 6027 | CA | ASP | P | 324 | 6.890 | −6.964 | 43.831 | 1.00 | 42.88 | C |
| ATOM | 6028 | C | ASP | P | 324 | 7.161 | −5.829 | 44.826 | 1.00 | 42.61 | C |
| ATOM | 6029 | O | ASP | P | 324 | 6.286 | −5.491 | 45.612 | 1.00 | 42.97 | O |
| ATOM | 6030 | CB | ASP | P | 324 | 7.003 | −8.330 | 44.539 | 1.00 | 42.97 | C |
| ATOM | 6031 | CG | ASP | P | 324 | 8.317 | −8.494 | 45.322 | 1.00 | 44.32 | C |
| ATOM | 6032 | OD1 | ASP | P | 324 | 9.326 | −7.828 | 44.982 | 1.00 | 45.99 | O |
| ATOM | 6033 | OD2 | ASP | P | 324 | 8.346 | −9.291 | 46.291 | 1.00 | 45.43 | O |
| ATOM | 6034 | N | ARG | P | 325 | 8.358 | −5.248 | 44.797 | 1.00 | 42.21 | N |
| ATOM | 6035 | CA | ARG | P | 325 | 8.678 | −4.151 | 45.714 | 1.00 | 42.57 | C |
| ATOM | 6036 | C | ARG | P | 325 | 7.873 | −2.880 | 45.447 | 1.00 | 42.30 | C |
| ATOM | 6037 | O | ARG | P | 325 | 7.667 | −2.085 | 46.364 | 1.00 | 42.49 | O |
| ATOM | 6038 | CB | ARG | P | 325 | 10.167 | −3.766 | 45.693 | 1.00 | 42.79 | C |
| ATOM | 6039 | CG | ARG | P | 325 | 11.185 | −4.887 | 45.669 | 1.00 | 44.41 | C |
| ATOM | 6040 | CD | ARG | P | 325 | 10.958 | −5.927 | 46.734 | 1.00 | 46.74 | C |
| ATOM | 6041 | NE | ARG | P | 325 | 12.221 | −6.446 | 47.239 | 1.00 | 48.19 | N |
| ATOM | 6042 | CZ | ARG | P | 325 | 12.402 | −7.689 | 47.665 | 1.00 | 49.38 | C |
| ATOM | 6043 | NH1 | ARG | P | 325 | 11.403 | −8.566 | 47.626 | 1.00 | 49.96 | N |
| ATOM | 6044 | NH2 | ARG | P | 325 | 13.593 | −8.055 | 48.118 | 1.00 | 49.84 | N |
| ATOM | 6045 | N | LEU | P | 326 | 7.448 | −2.675 | 44.200 | 1.00 | 41.79 | N |
| ATOM | 6046 | CA | LEU | P | 326 | 6.793 | −1.422 | 43.804 | 1.00 | 41.26 | C |
| ATOM | 6047 | C | LEU | P | 326 | 5.479 | −1.159 | 44.547 | 1.00 | 40.95 | C |
| ATOM | 6048 | O | LEU | P | 326 | 4.691 | −2.081 | 44.789 | 1.00 | 40.57 | O |
| ATOM | 6049 | CB | LEU | P | 326 | 6.558 | −1.371 | 42.290 | 1.00 | 41.12 | C |
| ATOM | 6050 | CG | LEU | P | 326 | 7.768 | −1.291 | 41.353 | 1.00 | 41.39 | C |
| ATOM | 6051 | CD1 | LEU | P | 326 | 7.354 | −1.694 | 39.958 | 1.00 | 41.93 | C |
| ATOM | 6052 | CD2 | LEU | P | 326 | 8.419 | 0.092 | 41.338 | 1.00 | 41.04 | C |
| ATOM | 6053 | N | ASP | P | 327 | 5.283 | 0.106 | 44.921 | 1.00 | 40.41 | N |
| ATOM | 6054 | CA | ASP | P | 327 | 4.027 | 0.592 | 45.477 | 1.00 | 39.97 | C |
| ATOM | 6055 | C | ASP | P | 327 | 3.293 | 1.410 | 44.418 | 1.00 | 39.70 | C |
| ATOM | 6056 | O | ASP | P | 327 | 3.795 | 1.562 | 43.304 | 1.00 | 39.62 | O |
| ATOM | 6057 | CB | ASP | P | 327 | 4.275 | 1.419 | 46.750 | 1.00 | 40.04 | C |
| ATOM | 6058 | CG | ASP | P | 327 | 5.088 | 2.707 | 46.501 | 1.00 | 40.37 | C |
| ATOM | 6059 | OD1 | ASP | P | 327 | 5.446 | 3.035 | 45.343 | 1.00 | 40.37 | O |
| ATOM | 6060 | OD2 | ASP | P | 327 | 5.375 | 3.399 | 47.499 | 1.00 | 39.76 | O |
| ATOM | 6061 | N | LYS | P | 328 | 2.120 | 1.937 | 44.764 | 1.00 | 39.39 | N |
| ATOM | 6062 | CA | LYS | P | 328 | 1.335 | 2.753 | 43.838 | 1.00 | 39.38 | C |
| ATOM | 6063 | C | LYS | P | 328 | 2.144 | 3.910 | 43.226 | 1.00 | 39.46 | C |
| ATOM | 6064 | O | LYS | P | 328 | 2.169 | 4.064 | 41.998 | 1.00 | 39.39 | O |
| ATOM | 6065 | CB | LYS | P | 328 | 0.073 | 3.291 | 44.524 | 1.00 | 39.13 | C |
| ATOM | 6066 | CG | LYS | P | 328 | −0.980 | 3.859 | 43.569 | 1.00 | 39.20 | C |
| ATOM | 6067 | CD | LYS | P | 328 | −2.141 | 4.541 | 44.298 | 1.00 | 39.79 | C |
| ATOM | 6068 | CE | LYS | P | 328 | −2.802 | 3.637 | 45.364 | 1.00 | 41.35 | C |
| ATOM | 6069 | NZ | LYS | P | 328 | −3.905 | 2.754 | 44.864 | 1.00 | 41.42 | N |
| ATOM | 6070 | N | ASP | P | 329 | 2.815 | 4.698 | 44.073 | 1.00 | 39.48 | N |
| ATOM | 6071 | CA | ASP | P | 329 | 3.412 | 5.971 | 43.639 | 1.00 | 39.54 | C |
| ATOM | 6072 | C | ASP | P | 329 | 4.631 | 5.815 | 42.735 | 1.00 | 39.64 | C |
| ATOM | 6073 | O | ASP | P | 329 | 4.810 | 6.596 | 41.795 | 1.00 | 39.92 | O |
| ATOM | 6074 | CB | ASP | P | 329 | 3.746 | 6.875 | 44.830 | 1.00 | 39.53 | C |
| ATOM | 6075 | CG | ASP | P | 329 | 2.505 | 7.469 | 45.491 | 1.00 | 39.77 | C |
| ATOM | 6076 | OD1 | ASP | P | 329 | 1.520 | 7.790 | 44.779 | 1.00 | 39.74 | O |
| ATOM | 6077 | OD2 | ASP | P | 329 | 2.525 | 7.633 | 46.732 | 1.00 | 39.69 | O |
| ATOM | 6078 | N | LEU | P | 330 | 5.466 | 4.816 | 43.011 | 1.00 | 39.40 | N |
| ATOM | 6079 | CA | LEU | P | 330 | 6.632 | 4.578 | 42.169 | 1.00 | 39.07 | C |
| ATOM | 6080 | C | LEU | P | 330 | 6.274 | 3.841 | 40.887 | 1.00 | 38.91 | C |
| ATOM | 6081 | O | LEU | P | 330 | 6.968 | 3.987 | 39.882 | 1.00 | 39.02 | O |
| ATOM | 6082 | CB | LEU | P | 330 | 7.749 | 3.853 | 42.926 | 1.00 | 38.94 | C |
| ATOM | 6083 | CG | LEU | P | 330 | 8.614 | 4.625 | 43.926 | 1.00 | 38.78 | C |
| ATOM | 6084 | CD1 | LEU | P | 330 | 9.956 | 3.928 | 44.092 | 1.00 | 38.21 | C |
| ATOM | 6085 | CD2 | LEU | P | 330 | 8.835 | 6.074 | 43.525 | 1.00 | 39.02 | C |
| ATOM | 6086 | N | THR | P | 331 | 5.204 | 3.048 | 40.924 | 1.00 | 38.72 | N |
| ATOM | 6087 | CA | THR | P | 331 | 4.699 | 2.388 | 39.718 | 1.00 | 38.70 | C |
| ATOM | 6088 | C | THR | P | 331 | 4.210 | 3.436 | 38.719 | 1.00 | 38.63 | C |
| ATOM | 6089 | O | THR | P | 331 | 4.654 | 3.448 | 37.574 | 1.00 | 38.50 | O |
| ATOM | 6090 | CB | THR | P | 331 | 3.574 | 1.383 | 40.029 | 1.00 | 38.71 | C |
| ATOM | 6091 | OG1 | THR | P | 331 | 4.075 | 0.370 | 40.902 | 1.00 | 39.11 | O |
| ATOM | 6092 | CG2 | THR | P | 331 | 3.069 | 0.718 | 38.760 | 1.00 | 38.36 | C |
| ATOM | 6093 | N | ILE | P | 332 | 3.322 | 4.323 | 39.165 | 1.00 | 38.47 | N |
| ATOM | 6094 | CA | ILE | P | 332 | 2.894 | 5.451 | 38.340 | 1.00 | 38.46 | C |
| ATOM | 6095 | C | ILE | P | 332 | 4.126 | 6.209 | 37.853 | 1.00 | 38.82 | C |
| ATOM | 6096 | O | ILE | P | 332 | 4.232 | 6.524 | 36.671 | 1.00 | 38.59 | O |
| ATOM | 6097 | CB | ILE | P | 332 | 1.919 | 6.403 | 39.095 | 1.00 | 38.30 | C |
| ATOM | 6098 | CG1 | ILE | P | 332 | 0.615 | 5.676 | 39.435 | 1.00 | 38.18 | C |
| ATOM | 6099 | CG2 | ILE | P | 332 | 1.600 | 7.648 | 38.258 | 1.00 | 37.59 | C |
| ATOM | 6100 | CD1 | ILE | P | 332 | −0.118 | 6.220 | 40.650 | 1.00 | 37.85 | C |
| ATOM | 6101 | N | GLY | P | 333 | 5.060 | 6.460 | 38.772 | 1.00 | 39.44 | N |
| ATOM | 6102 | CA | GLY | P | 333 | 6.278 | 7.215 | 38.491 | 1.00 | 40.21 | C |
| ATOM | 6103 | C | GLY | P | 333 | 7.163 | 6.591 | 37.433 | 1.00 | 40.99 | C |
| ATOM | 6104 | O | GLY | P | 333 | 7.661 | 7.285 | 36.542 | 1.00 | 41.00 | O |
| ATOM | 6105 | N | GLN | P | 334 | 7.339 | 5.275 | 37.507 | 1.00 | 41.60 | N |
| ATOM | 6106 | CA | GLN | P | 334 | 8.248 | 4.590 | 36.594 | 1.00 | 42.30 | C |
| ATOM | 6107 | C | GLN | P | 334 | 7.620 | 4.336 | 35.209 | 1.00 | 42.43 | C |
| ATOM | 6108 | O | GLN | P | 334 | 8.322 | 4.308 | 34.186 | 1.00 | 42.79 | O |
| ATOM | 6109 | CB | GLN | P | 334 | 8.818 | 3.331 | 37.257 | 1.00 | 42.27 | C |
| ATOM | 6110 | CG | GLN | P | 334 | 8.134 | 2.035 | 36.942 | 1.00 | 44.17 | C |
| ATOM | 6111 | CD | GLN | P | 334 | 8.909 | 1.241 | 35.911 | 1.00 | 46.67 | C |
| ATOM | 6112 | OE1 | GLN | P | 334 | 9.994 | 1.653 | 35.481 | 1.00 | 47.50 | O |
| ATOM | 6113 | NE2 | GLN | P | 334 | 8.363 | 0.097 | 35.508 | 1.00 | 47.21 | N |
| ATOM | 6114 | N | MET | P | 335 | 6.300 | 4.179 | 35.185 | 1.00 | 42.26 | N |
| ATOM | 6115 | CA | MET | P | 335 | 5.559 | 3.993 | 33.947 | 1.00 | 42.12 | C |
| ATOM | 6116 | C | MET | P | 335 | 5.408 | 5.320 | 33.219 | 1.00 | 42.10 | C |
| ATOM | 6117 | O | MET | P | 335 | 5.097 | 5.351 | 32.024 | 1.00 | 42.13 | O |
| ATOM | 6118 | CB | MET | P | 335 | 4.177 | 3.404 | 34.234 | 1.00 | 42.24 | C |
| ATOM | 6119 | CG | MET | P | 335 | 4.209 | 1.990 | 34.781 | 1.00 | 42.62 | C |
| ATOM | 6120 | SD | MET | P | 335 | 4.813 | 0.800 | 33.570 | 1.00 | 43.90 | S |
| ATOM | 6121 | CE | MET | P | 335 | 3.465 | 0.793 | 32.387 | 1.00 | 42.43 | C |
| ATOM | 6122 | N | GLN | P | 336 | 5.607 | 6.413 | 33.952 | 1.00 | 41.88 | N |
| ATOM | 6123 | CA | GLN | P | 336 | 5.624 | 7.744 | 33.359 | 1.00 | 41.70 | C |
| ATOM | 6124 | C | GLN | P | 336 | 7.050 | 8.127 | 32.984 | 1.00 | 41.71 | C |
| ATOM | 6125 | O | GLN | P | 336 | 7.306 | 9.248 | 32.550 | 1.00 | 41.79 | O |
| ATOM | 6126 | CB | GLN | P | 336 | 5.007 | 8.771 | 34.306 | 1.00 | 41.65 | C |
| ATOM | 6127 | CG | GLN | P | 336 | 3.495 | 8.687 | 34.360 | 1.00 | 41.64 | C |
| ATOM | 6128 | CD | GLN | P | 336 | 2.869 | 9.556 | 35.433 | 1.00 | 41.92 | C |
| ATOM | 6129 | OE1 | GLN | P | 336 | 1.715 | 9.950 | 35.309 | 1.00 | 42.46 | O |
| ATOM | 6130 | NE2 | GLN | P | 336 | 3.615 | 9.846 | 36.494 | 1.00 | 41.62 | N |
| ATOM | 6131 | N | GLY | P | 337 | 7.971 | 7.182 | 33.156 | 1.00 | 41.56 | N |
| ATOM | 6132 | CA | GLY | P | 337 | 9.355 | 7.355 | 32.742 | 1.00 | 41.41 | C |
| ATOM | 6133 | C | GLY | P | 337 | 10.095 | 8.440 | 33.495 | 1.00 | 41.19 | C |
| ATOM | 6134 | O | GLY | P | 337 | 10.867 | 9.194 | 32.904 | 1.00 | 41.30 | O |
| ATOM | 6135 | N | LYS | P | 338 | 9.876 | 8.508 | 34.803 | 1.00 | 40.89 | N |
| ATOM | 6136 | CA | LYS | P | 338 | 10.504 | 9.541 | 35.617 | 1.00 | 40.67 | C |
| ATOM | 6137 | C | LYS | P | 338 | 11.850 | 9.146 | 36.231 | 1.00 | 40.63 | C |
| ATOM | 6138 | O | LYS | P | 338 | 12.631 | 10.009 | 36.648 | 1.00 | 40.59 | O |
| ATOM | 6139 | CB | LYS | P | 338 | 9.529 | 10.074 | 36.660 | 1.00 | 40.37 | C |
| ATOM | 6140 | CG | LYS | P | 338 | 8.719 | 11.220 | 36.108 | 1.00 | 40.91 | C |
| ATOM | 6141 | CD | LYS | P | 338 | 7.818 | 11.844 | 37.138 | 1.00 | 41.89 | C |
| ATOM | 6142 | CE | LYS | P | 338 | 6.386 | 11.480 | 36.878 | 1.00 | 41.94 | C |
| ATOM | 6143 | NZ | LYS | P | 338 | 5.509 | 12.114 | 37.888 | 1.00 | 43.12 | N |
| ATOM | 6144 | N | PHE | P | 339 | 12.127 | 7.846 | 36.256 | 1.00 | 40.47 | N |
| ATOM | 6145 | CA | PHE | P | 339 | 13.384 | 7.347 | 36.780 | 1.00 | 40.47 | C |
| ATOM | 6146 | C | PHE | P | 339 | 13.744 | 6.032 | 36.130 | 1.00 | 40.49 | C |
| ATOM | 6147 | O | PHE | P | 339 | 13.027 | 5.566 | 35.270 | 1.00 | 40.38 | O |
| ATOM | 6148 | CB | PHE | P | 339 | 13.348 | 7.232 | 38.314 | 1.00 | 40.70 | C |
| ATOM | 6149 | CG | PHE | P | 339 | 12.297 | 6.289 | 38.849 | 1.00 | 40.38 | C |
| ATOM | 6150 | CD1 | PHE | P | 339 | 12.606 | 4.954 | 39.103 | 1.00 | 40.00 | C |
| ATOM | 6151 | CD2 | PHE | P | 339 | 11.017 | 6.747 | 39.140 | 1.00 | 40.09 | C |
| ATOM | 6152 | CE1 | PHE | P | 339 | 11.650 | 4.090 | 39.621 | 1.00 | 39.66 | C |
| ATOM | 6153 | CE2 | PHE | P | 339 | 10.054 | 5.891 | 39.655 | 1.00 | 40.13 | C |
| ATOM | 6154 | CZ | PHE | P | 339 | 10.374 | 4.560 | 39.900 | 1.00 | 40.28 | C |
| ATOM | 6155 | N | GLN | P | 340 | 14.877 | 5.458 | 36.524 | 1.00 | 41.18 | N |
| ATOM | 6156 | CA | GLN | P | 340 | 15.351 | 4.192 | 35.963 | 1.00 | 41.53 | C |
| ATOM | 6157 | C | GLN | P | 340 | 15.231 | 3.075 | 36.986 | 1.00 | 42.07 | C |
| ATOM | 6158 | O | GLN | P | 340 | 15.510 | 3.267 | 38.171 | 1.00 | 42.32 | O |
| ATOM | 6159 | CB | GLN | P | 340 | 16.801 | 4.305 | 35.487 | 1.00 | 41.46 | C |
| ATOM | 6160 | CG | GLN | P | 340 | 17.223 | 3.172 | 34.561 | 1.00 | 41.28 | C |
| ATOM | 6161 | CD | GLN | P | 340 | 18.715 | 3.125 | 34.285 | 1.00 | 40.94 | C |
| ATOM | 6162 | OE1 | GLN | P | 340 | 19.411 | 4.142 | 34.331 | 1.00 | 39.59 | O |
| ATOM | 6163 | NE2 | GLN | P | 340 | 19.212 | 1.930 | 33.989 | 1.00 | 39.61 | N |
| ATOM | 6164 | N | MET | P | 341 | 14.804 | 1.908 | 36.525 | 1.00 | 42.50 | N |
| ATOM | 6165 | CA | MET | P | 341 | 14.649 | 0.769 | 37.407 | 1.00 | 43.06 | C |
| ATOM | 6166 | C | MET | P | 341 | 15.313 | −0.446 | 36.788 | 1.00 | 43.22 | C |
| ATOM | 6167 | O | MET | P | 341 | 15.093 | −0.746 | 35.618 | 1.00 | 43.35 | O |
| ATOM | 6168 | CB | MET | P | 341 | 13.164 | 0.507 | 37.671 | 1.00 | 42.83 | C |
| ATOM | 6169 | CG | MET | P | 341 | 12.878 | −0.682 | 38.564 | 1.00 | 42.86 | C |
| ATOM | 6170 | SD | MET | P | 341 | 11.116 | −1.043 | 38.653 | 1.00 | 43.99 | S |
| ATOM | 6171 | CE | MET | P | 341 | 10.828 | −1.851 | 37.088 | 1.00 | 44.47 | C |
| ATOM | 6172 | N | GLN | P | 342 | 16.155 | −1.121 | 37.562 | 1.00 | 43.69 | N |
| ATOM | 6173 | CA | GLN | P | 342 | 16.624 | −2.449 | 37.178 | 1.00 | 44.21 | C |
| ATOM | 6174 | C | GLN | P | 342 | 16.169 | −3.440 | 38.229 | 1.00 | 44.35 | C |
| ATOM | 6175 | O | GLN | P | 342 | 16.174 | −3.145 | 39.427 | 1.00 | 44.22 | O |
| ATOM | 6176 | CB | GLN | P | 342 | 18.146 | −2.517 | 36.999 | 1.00 | 44.32 | C |
| ATOM | 6177 | CG | GLN | P | 342 | 18.756 | −1.493 | 36.026 | 1.00 | 45.44 | C |
| ATOM | 6178 | CD | GLN | P | 342 | 18.233 | −1.614 | 34.598 | 1.00 | 46.93 | C |
| ATOM | 6179 | OE1 | GLN | P | 342 | 17.980 | −0.603 | 33.929 | 1.00 | 47.04 | O |
| ATOM | 6180 | NE2 | GLN | P | 342 | 18.065 | −2.850 | 34.126 | 1.00 | 46.90 | N |
| ATOM | 6181 | N | VAL | P | 343 | 15.746 | −4.606 | 37.761 | 1.00 | 44.71 | N |
| ATOM | 6182 | CA | VAL | P | 343 | 15.333 | −5.689 | 38.632 | 1.00 | 45.10 | C |
| ATOM | 6183 | C | VAL | P | 343 | 16.389 | −6.792 | 38.553 | 1.00 | 45.60 | C |
| ATOM | 6184 | O | VAL | P | 343 | 16.854 | −7.148 | 37.464 | 1.00 | 45.57 | O |
| ATOM | 6185 | CB | VAL | P | 343 | 13.935 | −6.207 | 38.234 | 1.00 | 44.91 | C |
| ATOM | 6186 | CG1 | VAL | P | 343 | 13.620 | −7.531 | 38.901 | 1.00 | 44.86 | C |
| ATOM | 6187 | CG2 | VAL | P | 343 | 12.871 | −5.171 | 38.576 | 1.00 | 45.20 | C |
| ATOM | 6188 | N | LEU | P | 344 | 16.786 | −7.307 | 39.712 | 1.00 | 46.17 | N |
| ATOM | 6189 | CA | LEU | P | 344 | 17.761 | −8.384 | 39.774 | 1.00 | 46.96 | C |
| ATOM | 6190 | C | LEU | P | 344 | 17.202 | −9.537 | 40.608 | 1.00 | 47.49 | C |
| ATOM | 6191 | O | LEU | P | 344 | 17.545 | −9.684 | 41.785 | 1.00 | 47.77 | O |
| ATOM | 6192 | CB | LEU | P | 344 | 19.088 | −7.871 | 40.343 | 1.00 | 47.03 | C |
| ATOM | 6193 | CG | LEU | P | 344 | 19.771 | −6.702 | 39.618 | 1.00 | 47.44 | C |
| ATOM | 6194 | CD1 | LEU | P | 344 | 20.944 | −6.180 | 40.425 | 1.00 | 47.49 | C |
| ATOM | 6195 | CD2 | LEU | P | 344 | 20.219 | −7.094 | 38.202 | 1.00 | 48.53 | C |
| ATOM | 6196 | N | PRO | P | 345 | 16.349 | −10.379 | 39.992 | 1.00 | 47.88 | N |
| ATOM | 6197 | CA | PRO | P | 345 | 15.571 | −11.389 | 40.725 | 1.00 | 48.04 | C |
| ATOM | 6198 | C | PRO | P | 345 | 16.430 | −12.528 | 41.285 | 1.00 | 48.17 | C |
| ATOM | 6199 | O | PRO | P | 345 | 15.934 | −13.371 | 42.033 | 1.00 | 48.25 | O |
| ATOM | 6200 | CB | PRO | P | 345 | 14.600 | −11.911 | 39.664 | 1.00 | 48.12 | C |
| ATOM | 6201 | CG | PRO | P | 345 | 15.342 | −11.739 | 38.373 | 1.00 | 48.22 | C |
| ATOM | 6202 | CD | PRO | P | 345 | 16.108 | −10.449 | 38.538 | 1.00 | 47.93 | C |
| ATOM | 6203 | N | GLN | P | 346 | 17.706 | −12.539 | 40.919 | 1.00 | 48.32 | N |
| ATOM | 6204 | CA | GLN | P | 346 | 18.671 | −13.496 | 41.449 | 1.00 | 48.44 | C |
| ATOM | 6205 | C | GLN | P | 346 | 19.191 | −13.099 | 42.837 | 1.00 | 48.24 | C |
| ATOM | 6206 | O | GLN | P | 346 | 19.990 | −13.817 | 43.431 | 1.00 | 48.56 | O |
| ATOM | 6207 | CB | GLN | P | 346 | 19.827 | −13.702 | 40.452 | 1.00 | 48.62 | C |
| ATOM | 6208 | CG | GLN | P | 346 | 19.923 | −12.632 | 39.335 | 1.00 | 49.59 | C |
| ATOM | 6209 | CD | GLN | P | 346 | 20.912 | −11.496 | 39.629 | 1.00 | 50.63 | C |
| ATOM | 6210 | OE1 | GLN | P | 346 | 21.646 | −11.522 | 40.623 | 1.00 | 50.90 | O |
| ATOM | 6211 | NE2 | GLN | P | 346 | 20.941 | −10.498 | 38.745 | 1.00 | 50.78 | N |
| ATOM | 6212 | N | CYS | P | 347 | 18.716 | −11.972 | 43.358 | 1.00 | 47.98 | N |
| ATOM | 6213 | CA | CYS | P | 347 | 19.177 | −11.458 | 44.649 | 1.00 | 47.89 | C |
| ATOM | 6214 | C | CYS | P | 347 | 18.144 | −11.565 | 45.751 | 1.00 | 47.29 | C |
| ATOM | 6215 | O | CYS | P | 347 | 16.947 | −11.658 | 45.484 | 1.00 | 47.46 | O |
| ATOM | 6216 | CB | CYS | P | 347 | 19.575 | −9.996 | 44.517 | 1.00 | 47.95 | C |
| ATOM | 6217 | SG | CYS | P | 347 | 20.920 | −9.755 | 43.391 | 1.00 | 50.13 | S |
| ATOM | 6218 | N | GLY | P | 348 | 18.631 | −11.560 | 46.990 | 1.00 | 46.76 | N |
| ATOM | 6219 | CA | GLY | P | 348 | 17.791 | −11.386 | 48.173 | 1.00 | 45.93 | C |
| ATOM | 6220 | C | GLY | P | 348 | 17.690 | −9.897 | 48.431 | 1.00 | 45.42 | C |
| ATOM | 6221 | O | GLY | P | 348 | 17.782 | −9.100 | 47.493 | 1.00 | 45.69 | O |
| ATOM | 6222 | N | HIS | P | 349 | 17.531 | −9.514 | 49.695 | 1.00 | 44.73 | N |
| ATOM | 6223 | CA | HIS | P | 349 | 17.369 | −8.103 | 50.059 | 1.00 | 44.01 | C |
| ATOM | 6224 | C | HIS | P | 349 | 18.645 | −7.262 | 49.927 | 1.00 | 43.64 | C |
| ATOM | 6225 | O | HIS | P | 349 | 18.581 | −6.103 | 49.529 | 1.00 | 43.88 | O |
| ATOM | 6226 | CB | HIS | P | 349 | 16.770 | −7.959 | 51.465 | 1.00 | 44.03 | C |
| ATOM | 6227 | CG | HIS | P | 349 | 16.580 | −6.539 | 51.893 | 1.00 | 43.92 | C |
| ATOM | 6228 | ND1 | HIS | P | 349 | 15.680 | −5.692 | 51.283 | 1.00 | 44.13 | N |
| ATOM | 6229 | CD2 | HIS | P | 349 | 17.190 | −5.810 | 52.855 | 1.00 | 44.51 | C |
| ATOM | 6230 | CE1 | HIS | P | 349 | 15.742 | −4.502 | 51.851 | 1.00 | 44.34 | C |
| ATOM | 6231 | NE2 | HIS | P | 349 | 16.647 | −4.547 | 52.812 | 1.00 | 44.95 | N |
| ATOM | 6232 | N | ALA | P | 350 | 19.793 | −7.832 | 50.267 | 1.00 | 43.21 | N |
| ATOM | 6233 | CA | ALA | P | 350 | 21.059 | −7.119 | 50.144 | 1.00 | 42.92 | C |
| ATOM | 6234 | C | ALA | P | 350 | 21.648 | −7.314 | 48.744 | 1.00 | 42.80 | C |
| ATOM | 6235 | O | ALA | P | 350 | 22.624 | −8.051 | 48.570 | 1.00 | 42.68 | O |
| ATOM | 6236 | CB | ALA | P | 350 | 22.032 | −7.581 | 51.216 | 1.00 | 42.90 | C |
| ATOM | 6237 | N | VAL | P | 351 | 21.058 | −6.633 | 47.759 | 1.00 | 42.60 | N |
| ATOM | 6238 | CA | VAL | P | 351 | 21.323 | −6.910 | 46.339 | 1.00 | 42.51 | C |
| ATOM | 6239 | C | VAL | P | 351 | 22.752 | −6.553 | 45.927 | 1.00 | 42.66 | C |
| ATOM | 6240 | O | VAL | P | 351 | 23.349 | −7.221 | 45.086 | 1.00 | 42.61 | O |
| ATOM | 6241 | CB | VAL | P | 351 | 20.213 | −6.311 | 45.380 | 1.00 | 42.59 | C |
| ATOM | 6242 | CG1 | VAL | P | 351 | 19.006 | −5.824 | 46.181 | 1.00 | 42.14 | C |
| ATOM | 6243 | CG2 | VAL | P | 351 | 20.735 | −5.210 | 44.471 | 1.00 | 41.56 | C |
| ATOM | 6244 | N | HIS | P | 352 | 23.288 | −5.511 | 46.556 | 1.00 | 42.81 | N |
| ATOM | 6245 | CA | HIS | P | 352 | 24.668 | −5.073 | 46.365 | 1.00 | 42.98 | C |
| ATOM | 6246 | C | HIS | P | 352 | 25.665 | −6.066 | 46.959 | 1.00 | 43.28 | C |
| ATOM | 6247 | O | HIS | P | 352 | 26.806 | −6.151 | 46.503 | 1.00 | 43.49 | O |
| ATOM | 6248 | CB | HIS | P | 352 | 24.872 | −3.693 | 46.998 | 1.00 | 42.89 | C |
| ATOM | 6249 | CG | HIS | P | 352 | 24.536 | −3.643 | 48.459 | 1.00 | 42.66 | C |
| ATOM | 6250 | ND1 | HIS | P | 352 | 25.457 | −3.299 | 49.424 | 1.00 | 42.04 | N |
| ATOM | 6251 | CD2 | HIS | P | 352 | 23.383 | −3.909 | 49.119 | 1.00 | 42.08 | C |
| ATOM | 6252 | CE1 | HIS | P | 352 | 24.884 | −3.348 | 50.613 | 1.00 | 42.13 | C |
| ATOM | 6253 | NE2 | HIS | P | 352 | 23.627 | −3.718 | 50.456 | 1.00 | 42.00 | N |
| ATOM | 6254 | N | GLU | P | 353 | 25.237 | −6.802 | 47.981 | 1.00 | 43.54 | N |
| ATOM | 6255 | CA | GLU | P | 353 | 26.059 | −7.859 | 48.552 | 1.00 | 43.93 | C |
| ATOM | 6256 | C | GLU | P | 353 | 25.900 | −9.155 | 47.772 | 1.00 | 44.18 | C |
| ATOM | 6257 | O | GLU | P | 353 | 26.848 | −9.924 | 47.656 | 1.00 | 44.32 | O |
| ATOM | 6258 | CB | GLU | P | 353 | 25.721 | −8.101 | 50.025 | 1.00 | 44.03 | C |
| ATOM | 6259 | CG | GLU | P | 353 | 26.188 | −7.009 | 50.993 | 1.00 | 44.46 | C |
| ATOM | 6260 | CD | GLU | P | 353 | 26.565 | −7.562 | 52.363 | 1.00 | 44.90 | C |
| ATOM | 6261 | OE1 | GLU | P | 353 | 27.253 | −8.607 | 52.405 | 1.00 | 44.58 | O |
| ATOM | 6262 | OE2 | GLU | P | 353 | 26.186 | −6.951 | 53.395 | 1.00 | 45.41 | O |
| ATOM | 6263 | N | ASP | P | 354 | 24.703 | −9.395 | 47.240 | 1.00 | 44.54 | N |
| ATOM | 6264 | CA | ASP | P | 354 | 24.406 | −10.647 | 46.536 | 1.00 | 44.92 | C |
| ATOM | 6265 | C | ASP | P | 354 | 24.891 | −10.658 | 45.091 | 1.00 | 45.07 | C |
| ATOM | 6266 | O | ASP | P | 354 | 25.256 | −11.708 | 44.567 | 1.00 | 45.03 | O |
| ATOM | 6267 | CB | ASP | P | 354 | 22.909 | −10.962 | 46.585 | 1.00 | 44.90 | C |
| ATOM | 6268 | CG | ASP | P | 354 | 22.391 | −11.105 | 48.003 | 1.00 | 45.81 | C |
| ATOM | 6269 | OD1 | ASP | P | 354 | 23.167 | −11.511 | 48.890 | 1.00 | 46.78 | O |
| ATOM | 6270 | OD2 | ASP | P | 354 | 21.204 | −10.805 | 48.242 | 1.00 | 47.22 | O |
| ATOM | 6271 | N | ALA | P | 355 | 24.882 | −9.494 | 44.449 | 1.00 | 45.22 | N |
| ATOM | 6272 | CA | ALA | P | 355 | 25.304 | −9.385 | 43.058 | 1.00 | 45.54 | C |
| ATOM | 6273 | C | ALA | P | 355 | 26.090 | −8.099 | 42.843 | 1.00 | 45.93 | C |
| ATOM | 6274 | O | ALA | P | 355 | 25.619 | −7.193 | 42.147 | 1.00 | 45.80 | O |
| ATOM | 6275 | CB | ALA | P | 355 | 24.104 | −9.444 | 42.127 | 1.00 | 45.45 | C |
| ATOM | 6276 | N | PRO | P | 356 | 27.307 | −8.023 | 43.420 | 1.00 | 46.26 | N |
| ATOM | 6277 | CA | PRO | P | 356 | 28.091 | −6.793 | 43.392 | 1.00 | 46.46 | C |
| ATOM | 6278 | C | PRO | P | 356 | 28.537 | −6.499 | 41.970 | 1.00 | 46.76 | C |
| ATOM | 6279 | O | PRO | P | 356 | 28.631 | −5.337 | 41.570 | 1.00 | 46.81 | O |
| ATOM | 6280 | CB | PRO | P | 356 | 29.302 | −7.130 | 44.264 | 1.00 | 46.46 | C |
| ATOM | 6281 | CG | PRO | P | 356 | 28.938 | −8.381 | 44.992 | 1.00 | 46.27 | C |
| ATOM | 6282 | CD | PRO | P | 356 | 28.035 | −9.112 | 44.092 | 1.00 | 46.16 | C |
| ATOM | 6283 | N | ASP | P | 357 | 28.790 | −7.573 | 41.227 | 1.00 | 47.08 | N |
| ATOM | 6284 | CA | ASP | P | 357 | 29.136 | −7.539 | 39.818 | 1.00 | 47.35 | C |
| ATOM | 6285 | C | ASP | P | 357 | 28.143 | −6.693 | 39.023 | 1.00 | 47.07 | C |
| ATOM | 6286 | O | ASP | P | 357 | 28.520 | −5.712 | 38.385 | 1.00 | 47.02 | O |
| ATOM | 6287 | CB | ASP | P | 357 | 29.143 | −8.977 | 39.290 | 1.00 | 47.71 | C |
| ATOM | 6288 | CG | ASP | P | 357 | 30.077 | −9.168 | 38.111 | 1.00 | 49.17 | C |
| ATOM | 6289 | OD1 | ASP | P | 357 | 30.175 | −8.240 | 37.270 | 1.00 | 50.29 | O |
| ATOM | 6290 | OD2 | ASP | P | 357 | 30.705 | −10.257 | 38.022 | 1.00 | 50.16 | O |
| ATOM | 6291 | N | LYS | P | 358 | 26.871 | −7.066 | 39.096 | 1.00 | 47.03 | N |
| ATOM | 6292 | CA | LYS | P | 358 | 25.820 | −6.428 | 38.303 | 1.00 | 47.06 | C |
| ATOM | 6293 | C | LYS | P | 358 | 25.326 | −5.081 | 38.831 | 1.00 | 46.57 | C |
| ATOM | 6294 | O | LYS | P | 358 | 24.832 | −4.261 | 38.057 | 1.00 | 46.66 | O |
| ATOM | 6295 | CB | LYS | P | 358 | 24.643 | −7.387 | 38.087 | 1.00 | 47.37 | C |
| ATOM | 6296 | CG | LYS | P | 358 | 24.981 | −8.543 | 37.145 | 1.00 | 48.52 | C |
| ATOM | 6297 | CD | LYS | P | 358 | 23.770 | −8.969 | 36.332 | 1.00 | 50.86 | C |
| ATOM | 6298 | CE | LYS | P | 358 | 24.124 | −10.092 | 35.366 | 1.00 | 52.31 | C |
| ATOM | 6299 | NZ | LYS | P | 358 | 25.157 | −9.682 | 34.363 | 1.00 | 53.75 | N |
| ATOM | 6300 | N | VAL | P | 359 | 25.445 | −4.856 | 40.135 | 1.00 | 45.96 | N |
| ATOM | 6301 | CA | VAL | P | 359 | 25.136 | −3.553 | 40.708 | 1.00 | 45.45 | C |
| ATOM | 6302 | C | VAL | P | 359 | 26.189 | −2.541 | 40.238 | 1.00 | 45.29 | C |
| ATOM | 6303 | O | VAL | P | 359 | 25.854 | −1.408 | 39.880 | 1.00 | 45.26 | O |
| ATOM | 6304 | CB | VAL | P | 359 | 25.046 | −3.605 | 42.256 | 1.00 | 45.48 | C |
| ATOM | 6305 | CG1 | VAL | P | 359 | 24.779 | −2.224 | 42.839 | 1.00 | 45.00 | C |
| ATOM | 6306 | CG2 | VAL | P | 359 | 23.950 | −4.554 | 42.686 | 1.00 | 45.38 | C |
| ATOM | 6307 | N | ALA | P | 360 | 27.454 | −2.962 | 40.220 | 1.00 | 44.91 | N |
| ATOM | 6308 | CA | ALA | P | 360 | 28.536 | −2.129 | 39.696 | 1.00 | 44.51 | C |
| ATOM | 6309 | C | ALA | P | 360 | 28.267 | −1.779 | 38.240 | 1.00 | 44.43 | C |
| ATOM | 6310 | O | ALA | P | 360 | 28.373 | −0.618 | 37.846 | 1.00 | 44.40 | O |
| ATOM | 6311 | CB | ALA | P | 360 | 29.863 | −2.838 | 39.827 | 1.00 | 44.37 | C |
| ATOM | 6312 | N | GLU | P | 361 | 27.900 | −2.796 | 37.460 | 1.00 | 44.32 | N |
| ATOM | 6313 | CA | GLU | P | 361 | 27.589 | −2.657 | 36.037 | 1.00 | 44.08 | C |
| ATOM | 6314 | C | GLU | P | 361 | 26.460 | −1.647 | 35.760 | 1.00 | 43.61 | C |
| ATOM | 6315 | O | GLU | P | 361 | 26.539 | −0.864 | 34.804 | 1.00 | 43.62 | O |
| ATOM | 6316 | CB | GLU | P | 361 | 27.225 | −4.024 | 35.451 | 1.00 | 44.11 | C |
| ATOM | 6317 | CG | GLU | P | 361 | 27.398 | −4.121 | 33.946 | 1.00 | 45.76 | C |
| ATOM | 6318 | CD | GLU | P | 361 | 26.220 | −4.804 | 33.265 | 1.00 | 48.11 | C |
| ATOM | 6319 | OE1 | GLU | P | 361 | 26.359 | −5.984 | 32.857 | 1.00 | 48.79 | O |
| ATOM | 6320 | OE2 | GLU | P | 361 | 25.151 | −4.158 | 33.146 | 1.00 | 48.83 | O |
| ATOM | 6321 | N | ALA | P | 362 | 25.419 | −1.671 | 36.597 | 1.00 | 42.94 | N |
| ATOM | 6322 | CA | ALA | P | 362 | 24.247 | −0.809 | 36.420 | 1.00 | 42.15 | C |
| ATOM | 6323 | C | ALA | P | 362 | 24.586 | 0.645 | 36.711 | 1.00 | 41.71 | C |
| ATOM | 6324 | O | ALA | P | 362 | 24.215 | 1.541 | 35.939 | 1.00 | 41.70 | O |
| ATOM | 6325 | CB | ALA | P | 362 | 23.100 | −1.273 | 37.302 | 1.00 | 42.01 | C |
| ATOM | 6326 | N | VAL | P | 363 | 25.292 | 0.857 | 37.825 | 1.00 | 40.93 | N |
| ATOM | 6327 | CA | VAL | P | 363 | 25.735 | 2.182 | 38.250 | 1.00 | 40.13 | C |
| ATOM | 6328 | C | VAL | P | 363 | 26.793 | 2.706 | 37.282 | 1.00 | 39.72 | C |
| ATOM | 6329 | O | VAL | P | 363 | 26.853 | 3.903 | 37.017 | 1.00 | 39.54 | O |
| ATOM | 6330 | CB | VAL | P | 363 | 26.296 | 2.170 | 39.697 | 1.00 | 40.07 | C |
| ATOM | 6331 | CG1 | VAL | P | 363 | 26.762 | 3.565 | 40.115 | 1.00 | 40.27 | C |
| ATOM | 6332 | CG2 | VAL | P | 363 | 25.267 | 1.665 | 40.674 | 1.00 | 39.26 | C |
| ATOM | 6333 | N | ALA | P | 364 | 27.615 | 1.802 | 36.754 | 1.00 | 39.40 | N |
| ATOM | 6334 | CA | ALA | P | 364 | 28.644 | 2.165 | 35.774 | 1.00 | 39.15 | C |
| ATOM | 6335 | C | ALA | P | 364 | 28.006 | 2.695 | 34.499 | 1.00 | 38.78 | C |
| ATOM | 6336 | O | ALA | P | 364 | 28.325 | 3.795 | 34.051 | 1.00 | 38.92 | O |
| ATOM | 6337 | CB | ALA | P | 364 | 29.558 | 0.976 | 35.467 | 1.00 | 39.22 | C |
| ATOM | 6338 | N | THR | P | 365 | 27.092 | 1.914 | 33.933 | 1.00 | 38.31 | N |
| ATOM | 6339 | CA | THR | P | 365 | 26.336 | 2.335 | 32.760 | 1.00 | 37.83 | C |
| ATOM | 6340 | C | THR | P | 365 | 25.646 | 3.673 | 33.030 | 1.00 | 37.47 | C |
| ATOM | 6341 | O | THR | P | 365 | 25.784 | 4.609 | 32.254 | 1.00 | 37.81 | O |
| ATOM | 6342 | CB | THR | P | 365 | 25.319 | 1.254 | 32.339 | 1.00 | 37.82 | C |
| ATOM | 6343 | OG1 | THR | P | 365 | 26.021 | 0.038 | 32.051 | 1.00 | 37.41 | O |
| ATOM | 6344 | CG2 | THR | P | 365 | 24.526 | 1.687 | 31.109 | 1.00 | 37.58 | C |
| ATOM | 6345 | N | PHE | P | 366 | 24.936 | 3.760 | 34.145 | 1.00 | 36.94 | N |
| ATOM | 6346 | CA | PHE | P | 366 | 24.282 | 4.995 | 34.560 | 1.00 | 36.61 | C |
| ATOM | 6347 | C | PHE | P | 366 | 25.255 | 6.177 | 34.480 | 1.00 | 36.71 | C |
| ATOM | 6348 | O | PHE | P | 366 | 24.954 | 7.194 | 33.851 | 1.00 | 36.83 | O |
| ATOM | 6349 | CB | PHE | P | 366 | 23.725 | 4.825 | 35.984 | 1.00 | 36.22 | C |
| ATOM | 6350 | CG | PHE | P | 366 | 23.055 | 6.050 | 36.543 | 1.00 | 35.18 | C |
| ATOM | 6351 | CD1 | PHE | P | 366 | 21.689 | 6.254 | 36.360 | 1.00 | 34.38 | C |
| ATOM | 6352 | CD2 | PHE | P | 366 | 23.781 | 6.979 | 37.286 | 1.00 | 35.02 | C |
| ATOM | 6353 | CE1 | PHE | P | 366 | 21.055 | 7.375 | 36.882 | 1.00 | 34.19 | C |
| ATOM | 6354 | CE2 | PHE | P | 366 | 23.160 | 8.108 | 37.820 | 1.00 | 35.05 | C |
| ATOM | 6355 | CZ | PHE | P | 366 | 21.789 | 8.305 | 37.615 | 1.00 | 35.51 | C |
| ATOM | 6356 | N | LEU | P | 367 | 26.420 | 6.029 | 35.105 | 1.00 | 36.58 | N |
| ATOM | 6357 | CA | LEU | P | 367 | 27.410 | 7.099 | 35.150 | 1.00 | 36.67 | C |
| ATOM | 6358 | C | LEU | P | 367 | 27.928 | 7.497 | 33.762 | 1.00 | 36.95 | C |
| ATOM | 6359 | O | LEU | P | 367 | 27.985 | 8.690 | 33.445 | 1.00 | 36.96 | O |
| ATOM | 6360 | CB | LEU | P | 367 | 28.577 | 6.735 | 36.084 | 1.00 | 36.44 | C |
| ATOM | 6361 | CG | LEU | P | 367 | 28.313 | 6.624 | 37.596 | 1.00 | 35.59 | C |
| ATOM | 6362 | CD1 | LEU | P | 367 | 29.457 | 5.895 | 38.278 | 1.00 | 34.37 | C |
| ATOM | 6363 | CD2 | LEU | P | 367 | 28.054 | 7.970 | 38.267 | 1.00 | 34.48 | C |
| ATOM | 6364 | N | ILE | P | 368 | 28.294 | 6.510 | 32.940 | 1.00 | 37.09 | N |
| ATOM | 6365 | CA | ILE | P | 368 | 28.843 | 6.792 | 31.606 | 1.00 | 37.32 | C |
| ATOM | 6366 | C | ILE | P | 368 | 27.793 | 7.472 | 30.720 | 1.00 | 37.71 | C |
| ATOM | 6367 | O | ILE | P | 368 | 28.098 | 8.412 | 29.984 | 1.00 | 37.63 | O |
| ATOM | 6368 | CB | ILE | P | 368 | 29.432 | 5.523 | 30.917 | 1.00 | 37.31 | C |
| ATOM | 6369 | CG1 | ILE | P | 368 | 30.617 | 4.973 | 31.724 | 1.00 | 37.29 | C |
| ATOM | 6370 | CG2 | ILE | P | 368 | 29.884 | 5.836 | 29.477 | 1.00 | 36.67 | C |
| ATOM | 6371 | CD1 | ILE | P | 368 | 30.998 | 3.532 | 31.394 | 1.00 | 37.07 | C |
| ATOM | 6372 | N | ARG | P | 369 | 26.556 | 7.000 | 30.833 | 1.00 | 38.21 | N |
| ATOM | 6373 | CA | ARG | P | 369 | 25.440 | 7.471 | 30.026 | 1.00 | 38.77 | C |
| ATOM | 6374 | C | ARG | P | 369 | 25.081 | 8.940 | 30.307 | 1.00 | 39.23 | C |
| ATOM | 6375 | O | ARG | P | 369 | 24.772 | 9.694 | 29.384 | 1.00 | 39.37 | O |
| ATOM | 6376 | CB | ARG | P | 369 | 24.241 | 6.552 | 30.251 | 1.00 | 38.67 | C |
| ATOM | 6377 | CG | ARG | P | 369 | 23.193 | 6.601 | 29.176 | 1.00 | 39.39 | C |
| ATOM | 6378 | CD | ARG | P | 369 | 22.017 | 5.714 | 29.523 | 1.00 | 40.02 | C |
| ATOM | 6379 | NE | ARG | P | 369 | 20.785 | 6.237 | 28.945 | 1.00 | 41.00 | N |
| ATOM | 6380 | CZ | ARG | P | 369 | 19.825 | 6.851 | 29.630 | 1.00 | 41.39 | C |
| ATOM | 6381 | NH1 | ARG | P | 369 | 19.923 | 7.010 | 30.947 | 1.00 | 41.08 | N |
| ATOM | 6382 | NH2 | ARG | P | 369 | 18.746 | 7.287 | 28.991 | 1.00 | 41.84 | N |
| ATOM | 6383 | N | HIS | P | 370 | 25.135 | 9.343 | 31.574 | 1.00 | 39.78 | N |
| ATOM | 6384 | CA | HIS | P | 370 | 24.939 | 10.744 | 31.952 | 1.00 | 40.24 | C |
| ATOM | 6385 | C | HIS | P | 370 | 26.251 | 11.527 | 31.926 | 1.00 | 40.73 | C |
| ATOM | 6386 | O | HIS | P | 370 | 26.285 | 12.724 | 32.239 | 1.00 | 40.87 | O |
| ATOM | 6387 | CB | HIS | P | 370 | 24.306 | 10.838 | 33.336 | 1.00 | 40.12 | C |
| ATOM | 6388 | CG | HIS | P | 370 | 22.912 | 10.303 | 33.399 | 1.00 | 40.08 | C |
| ATOM | 6389 | ND1 | HIS | P | 370 | 21.799 | 11.112 | 33.298 | 1.00 | 40.27 | N |
| ATOM | 6390 | CD2 | HIS | P | 370 | 22.449 | 9.040 | 33.549 | 1.00 | 39.28 | C |
| ATOM | 6391 | CE1 | HIS | P | 370 | 20.710 | 10.370 | 33.387 | 1.00 | 39.81 | C |
| ATOM | 6392 | NE2 | HIS | P | 370 | 21.077 | 9.110 | 33.543 | 1.00 | 39.45 | N |
| ATOM | 6393 | N | ARG | P | 371 | 27.329 | 10.840 | 31.555 | 1.00 | 41.32 | N |
| ATOM | 6394 | CA | ARG | P | 371 | 28.662 | 11.443 | 31.435 | 1.00 | 41.83 | C |
| ATOM | 6395 | C | ARG | P | 371 | 29.130 | 12.054 | 32.758 | 1.00 | 41.90 | C |
| ATOM | 6396 | O | ARG | P | 371 | 29.774 | 13.101 | 32.774 | 1.00 | 41.98 | O |
| ATOM | 6397 | CB | ARG | P | 371 | 28.699 | 12.460 | 30.288 | 1.00 | 41.84 | C |
| ATOM | 6398 | CG | ARG | P | 371 | 28.343 | 11.859 | 28.932 | 1.00 | 42.94 | C |
| ATOM | 6399 | CD | ARG | P | 371 | 27.912 | 12.938 | 27.948 | 1.00 | 45.22 | C |
| ATOM | 6400 | NE | ARG | P | 371 | 28.418 | 12.712 | 26.591 | 1.00 | 46.59 | N |
| ATOM | 6401 | CZ | ARG | P | 371 | 29.707 | 12.767 | 26.242 | 1.00 | 47.13 | C |
| ATOM | 6402 | NH1 | ARG | P | 371 | 30.648 | 13.016 | 27.151 | 1.00 | 46.99 | N |
| ATOM | 6403 | NH2 | ARG | P | 371 | 30.062 | 12.558 | 24.979 | 1.00 | 47.25 | N |
| ATOM | 6404 | N | PHE | P | 372 | 28.783 | 11.382 | 33.857 | 1.00 | 42.00 | N |
| ATOM | 6405 | CA | PHE | P | 372 | 29.242 | 11.747 | 35.189 | 1.00 | 42.11 | C |
| ATOM | 6406 | C | PHE | P | 372 | 30.613 | 11.142 | 35.464 | 1.00 | 42.41 | C |
| ATOM | 6407 | O | PHE | P | 372 | 31.354 | 11.633 | 36.313 | 1.00 | 42.57 | O |
| ATOM | 6408 | CB | PHE | P | 372 | 28.268 | 11.255 | 36.260 | 1.00 | 42.12 | C |
| ATOM | 6409 | CG | PHE | P | 372 | 26.918 | 11.918 | 36.229 | 1.00 | 42.08 | C |
| ATOM | 6410 | CD1 | PHE | P | 372 | 26.770 | 13.237 | 35.806 | 1.00 | 41.78 | C |
| ATOM | 6411 | CD2 | PHE | P | 372 | 25.790 | 11.226 | 36.678 | 1.00 | 42.44 | C |
| ATOM | 6412 | CE1 | PHE | P | 372 | 25.514 | 13.849 | 35.793 | 1.00 | 41.88 | C |
| ATOM | 6413 | CE2 | PHE | P | 372 | 24.525 | 11.830 | 36.682 | 1.00 | 42.46 | C |
| ATOM | 6414 | CZ | PHE | P | 372 | 24.388 | 13.147 | 36.234 | 1.00 | 42.23 | C |
| ATOM | 6415 | N | ALA | P | 373 | 30.939 | 10.064 | 34.760 | 1.00 | 42.69 | N |
| ATOM | 6416 | CA | ALA | P | 373 | 32.229 | 9.404 | 34.925 | 1.00 | 43.03 | C |
| ATOM | 6417 | C | ALA | P | 373 | 32.794 | 8.944 | 33.584 | 1.00 | 43.35 | C |
| ATOM | 6418 | O | ALA | P | 373 | 32.149 | 9.086 | 32.549 | 1.00 | 43.41 | O |
| ATOM | 6419 | CB | ALA | P | 373 | 32.110 | 8.231 | 35.892 | 1.00 | 42.92 | C |
| ATOM | 6420 | N | GLU | P | 374 | 34.008 | 8.404 | 33.621 | 1.00 | 43.85 | N |
| ATOM | 6421 | CA | GLU | P | 374 | 34.682 | 7.874 | 32.444 | 1.00 | 44.28 | C |
| ATOM | 6422 | C | GLU | P | 374 | 34.946 | 6.383 | 32.656 | 1.00 | 44.43 | C |
| ATOM | 6423 | O | GLU | P | 374 | 35.264 | 5.966 | 33.773 | 1.00 | 44.43 | O |
| ATOM | 6424 | CB | GLU | P | 374 | 36.012 | 8.604 | 32.223 | 1.00 | 44.42 | C |
| ATOM | 6425 | CG | GLU | P | 374 | 35.891 | 10.110 | 32.045 | 1.00 | 45.23 | C |
| ATOM | 6426 | CD | GLU | P | 374 | 35.670 | 10.511 | 30.595 | 1.00 | 46.76 | C |
| ATOM | 6427 | OE1 | GLU | P | 374 | 36.621 | 10.386 | 29.795 | 1.00 | 46.57 | O |
| ATOM | 6428 | OE2 | GLU | P | 374 | 34.549 | 10.960 | 30.258 | 1.00 | 47.55 | O |
| ATOM | 6429 | N | PRO | P | 375 | 34.802 | 5.571 | 31.593 | 1.00 | 44.63 | N |
| ATOM | 6430 | CA | PRO | P | 375 | 35.146 | 4.145 | 31.675 | 1.00 | 44.88 | C |
| ATOM | 6431 | C | PRO | P | 375 | 36.662 | 3.916 | 31.706 | 1.00 | 45.08 | C |
| ATOM | 6432 | O | PRO | P | 375 | 37.422 | 4.816 | 31.343 | 1.00 | 45.22 | O |
| ATOM | 6433 | CB | PRO | P | 375 | 34.556 | 3.573 | 30.385 | 1.00 | 44.92 | C |
| ATOM | 6434 | CG | PRO | P | 375 | 34.561 | 4.722 | 29.433 | 1.00 | 44.85 | C |
| ATOM | 6435 | CD | PRO | P | 375 | 34.285 | 5.941 | 30.262 | 1.00 | 44.62 | C |
| ATOM | 6436 | N | ILE | P | 376 | 37.091 | 2.728 | 32.132 | 1.00 | 45.18 | N |
| ATOM | 6437 | CA | ILE | P | 376 | 38.515 | 2.369 | 32.144 | 1.00 | 45.32 | C |
| ATOM | 6438 | C | ILE | P | 376 | 39.065 | 2.207 | 30.718 | 1.00 | 45.44 | C |
| ATOM | 6439 | O | ILE | P | 376 | 40.121 | 2.740 | 30.372 | 1.00 | 45.46 | O |
| ATOM | 6440 | CB | ILE | P | 376 | 38.787 | 1.087 | 32.994 | 1.00 | 45.35 | C |
| ATOM | 6441 | CG1 | ILE | P | 376 | 38.425 | 1.315 | 34.474 | 1.00 | 45.33 | C |
| ATOM | 6442 | CG2 | ILE | P | 376 | 40.239 | 0.606 | 32.839 | 1.00 | 45.06 | C |
| ATOM | 6443 | CD1 | ILE | P | 376 | 39.297 | 2.342 | 35.224 | 1.00 | 44.53 | C |
| TER | 6444 | | ILE | P | 376 | | | | | | |
| HETATM | 6445 | O | HOH | A | 1002 | −12.171 | 1.038 | 13.804 | 1.00 | 22.46 | O |
| HETATM | 6446 | O | HOH | A | 1005 | −11.004 | −3.635 | 0.238 | 1.00 | 20.02 | O |
| HETATM | 6447 | O | HOH | A | 1014 | −34.526 | −3.343 | 2.446 | 1.00 | 39.70 | O |
| HETATM | 6448 | O | HOH | A | 1017 | −20.539 | −16.425 | 6.363 | 1.00 | 40.87 | O |
| HETATM | 6449 | O | HOH | A | 1020 | −11.079 | −9.308 | −6.941 | 1.00 | 55.56 | O |
| HETATM | 6450 | O | HOH | A | 1026 | −26.379 | −26.452 | −4.636 | 1.00 | 48.28 | O |
| HETATM | 6451 | O | HOH | A | 1034 | −16.997 | −1.802 | −2.885 | 1.00 | 34.28 | O |
| HETATM | 6452 | O | HOH | A | 1042 | −22.612 | −7.451 | 32.773 | 1.00 | 41.78 | O |
| HETATM | 6453 | O | HOH | A | 1043 | −42.916 | −12.284 | 7.205 | 1.00 | 47.46 | O |
| HETATM | 6454 | O | HOH | A | 1055 | −32.632 | −0.812 | 21.218 | 1.00 | 35.96 | O |
| HETATM | 6455 | O | HOH | A | 1056 | −17.001 | −6.346 | 40.914 | 1.00 | 49.92 | O |
| HETATM | 6456 | O | HOH | C | 1001 | −9.495 | −6.807 | 14.102 | 1.00 | 22.50 | O |
| HETATM | 6457 | O | HOH | C | 1006 | 13.601 | 16.897 | 25.354 | 1.00 | 22.57 | O |
| HETATM | 6458 | O | HOH | C | 1007 | 19.407 | 7.179 | 18.349 | 1.00 | 30.19 | O |
| HETATM | 6459 | O | HOH | C | 1009 | −6.142 | −9.065 | 34.079 | 1.00 | 37.59 | O |
| HETATM | 6460 | O | HOH | C | 1010 | 11.256 | −6.011 | 15.760 | 1.00 | 26.05 | O |
| HETATM | 6461 | O | HOH | C | 1011 | 7.120 | −9.704 | 31.696 | 1.00 | 14.73 | O |
| HETATM | 6462 | O | HOH | C | 1012 | −17.811 | −3.950 | 15.370 | 1.00 | 24.88 | O |
| HETATM | 6463 | O | HOH | C | 1013 | 2.458 | 23.281 | 16.202 | 1.00 | 38.46 | O |
| HETATM | 6464 | O | HOH | C | 1015 | 4.132 | −5.268 | 40.109 | 1.00 | 48.17 | O |
| HETATM | 6465 | O | HOH | C | 1016 | 6.139 | 1.725 | 30.812 | 1.00 | 31.90 | O |
| HETATM | 6466 | O | HOH | C | 1018 | 5.527 | 0.081 | 18.270 | 1.00 | 29.50 | O |
| HETATM | 6467 | O | HOH | C | 1021 | 8.283 | 20.403 | 24.294 | 1.00 | 37.91 | O |
| HETATM | 6468 | O | HOH | C | 1022 | −13.024 | 3.287 | 13.598 | 1.00 | 20.06 | O |
| HETATM | 6469 | O | HOH | C | 1023 | 13.178 | −3.317 | 25.126 | 1.00 | 25.09 | O |
| HETATM | 6470 | O | HOH | C | 1024 | 1.380 | −6.448 | 12.484 | 1.00 | 32.63 | O |
| HETATM | 6471 | O | HOH | C | 1025 | −6.685 | −3.027 | 9.517 | 1.00 | 24.13 | O |
| HETATM | 6472 | O | HOH | C | 1027 | −15.939 | −9.385 | 23.020 | 1.00 | 37.04 | O |
| HETATM | 6473 | O | HOH | C | 1028 | 13.436 | −2.802 | 34.192 | 1.00 | 24.99 | O |
| HETATM | 6474 | O | HOH | C | 1029 | −1.193 | −4.904 | 9.228 | 1.00 | 41.36 | O |
| HETATM | 6475 | O | HOH | C | 1030 | 7.527 | 12.935 | 29.586 | 1.00 | 24.83 | O |
| HETATM | 6476 | O | HOH | C | 1031 | 19.798 | 5.502 | 21.778 | 1.00 | 43.15 | O |
| HETATM | 6477 | O | HOH | C | 1032 | −5.005 | −2.884 | 11.682 | 1.00 | 27.11 | O |
| HETATM | 6478 | O | HOH | C | 1035 | −0.077 | −8.819 | 7.205 | 1.00 | 27.90 | O |
| HETATM | 6479 | O | HOH | C | 1036 | −18.203 | 4.591 | 18.733 | 1.00 | 32.74 | O |
| HETATM | 6480 | O | HOH | C | 1037 | 18.472 | 0.374 | 18.104 | 1.00 | 38.08 | O |
| HETATM | 6481 | O | HOH | C | 1038 | 14.270 | 1.543 | 33.255 | 1.00 | 41.09 | O |
| HETATM | 6482 | O | HOH | C | 1040 | 16.156 | −4.971 | 25.116 | 1.00 | 27.49 | O |
| HETATM | 6483 | O | HOH | C | 1045 | 19.948 | −6.436 | 19.143 | 1.00 | 21.90 | O |
| HETATM | 6484 | O | HOH | C | 1047 | 5.376 | −10.277 | 38.112 | 1.00 | 34.03 | O |
| HETATM | 6485 | O | HOH | C | 1048 | 12.836 | 10.742 | 15.126 | 1.00 | 23.44 | O |
| HETATM | 6486 | O | HOH | C | 1050 | 11.031 | −2.164 | 6.303 | 1.00 | 28.60 | O |
| HETATM | 6487 | O | HOH | C | 1051 | 16.539 | −2.140 | 25.817 | 1.00 | 33.46 | O |
| HETATM | 6488 | O | HOH | C | 1052 | 6.011 | 0.564 | 4.142 | 1.00 | 30.66 | O |
| HETATM | 6489 | O | HOH | C | 1054 | −0.983 | −10.487 | 33.712 | 1.00 | 24.54 | O |
| HETATM | 6490 | O | HOH | C | 1062 | −12.616 | −10.335 | 19.878 | 1.00 | 41.98 | O |
| HETATM | 6491 | O | HOH | P | 1003 | 24.585 | −21.013 | 71.049 | 1.00 | 47.54 | O |
| HETATM | 6492 | O | HOH | P | 1004 | 16.133 | −0.202 | 31.887 | 1.00 | 34.10 | O |
| HETATM | 6493 | O | HOH | P | 1008 | 15.403 | 10.863 | 37.480 | 1.00 | 17.57 | O |
| HETATM | 6494 | O | HOH | P | 1019 | 0.875 | 1.417 | 47.120 | 1.00 | 23.67 | O |
| HETATM | 6495 | O | HOH | P | 1033 | 15.221 | −4.700 | 34.498 | 1.00 | 23.71 | O |
| HETATM | 6496 | O | HOH | P | 1039 | 27.466 | 2.495 | 53.931 | 1.00 | 38.50 | O |
| HETATM | 6497 | O | HOH | P | 1041 | 11.675 | 12.564 | 38.600 | 1.00 | 32.43 | O |
| HETATM | 6498 | O | HOH | P | 1044 | 32.949 | −1.037 | 59.923 | 1.00 | 28.34 | O |
| HETATM | 6499 | O | HOH | P | 1046 | 16.779 | −20.012 | 53.349 | 1.00 | 50.68 | O |
| HETATM | 6500 | O | HOH | P | 1049 | 6.868 | −12.216 | 60.170 | 1.00 | 34.64 | O |
| HETATM | 6501 | O | HOH | P | 1053 | 7.738 | 3.825 | 63.688 | 1.00 | 32.73 | O |
| HETATM | 6502 | O | HOH | P | 1057 | 5.056 | 8.668 | 48.508 | 1.00 | 43.12 | O |
| HETATM | 6503 | O | HOH | P | 1058 | 11.587 | 1.730 | 69.042 | 1.00 | 32.04 | O |
| HETATM | 6504 | O | HOH | P | 1059 | 29.178 | −4.951 | 48.322 | 1.00 | 51.34 | O |
| HETATM | 6505 | O | HOH | P | 1060 | 24.458 | 8.600 | 60.529 | 1.00 | 43.50 | O |
| HETATM | 6506 | O | HOH | P | 1061 | 30.478 | −6.543 | 51.202 | 1.00 | 41.07 | O |

| | | | | | | | | | | | |
|---|---|---|---|---|---|---|---|---|---|---|---|
| MASTER | 413 | 0 | 0 | 47 | 29 | 0 | 0 | 6 6503 | 3 | 0 | 66 |
| END | | | | | | | | | | | |

What is claimed is:

1. A composition comprising a crystal of PME-1.

2. The composition of claim 1, wherein said crystal has space group P3₁21.

3. The composition of claim 2, wherein said crystal has unit cell dimensions, ±2%, of a=b=82.5 Å, c=90.8 Å, α=β=90°, γ=120°.

4. The composition of claim 4, wherein said crystal comprises one protein molecules in each asymmetric unit.

5. The composition of claim 1 wherein the crystal diffracts X-rays for a determination of structure coordinates to a resolution of a value equal to or less than about 5.0 angstroms.

6. The composition of claim 1 wherein said crystal has the structure defined by the coordinates of Appendix A.

7. The composition of claim 1, wherein a methionine of PME-1 is replaced with selenomethionine in the amino acid sequence of said PME-1.

8. The composition of claim 1, wherein PME-1 is complexed with PP2A, wherein said PP2A comprises an A subunit and a C subunit.

9. The composition of claim 8, wherein said crystal has space group C2.

10. The composition of claim 8, wherein said crystal has unit cell dimensions, ±2%, of a=129.3 Å, b=54.8 Å, c=125.2 Å, α=90°, β=111°, γ90°.

11. The composition of claim 10, wherein said unit cell comprises one complex per asymmetric unit.

12. The composition of claim 8 wherein the crystal diffracts X-rays for a determination of structure coordinates to a resolution of a value equal to or less than 5.0 angstroms.

13. The composition of claim 8, wherein said PP2A A subunit is AΔN.

14. The composition of claim 8 wherein said crystal has the structure defined by the coordinates of Appendix B.

15. A method for preparing a PME-1 modulating compound comprising:
applying a three-dimensional molecular modeling algorithm to the atomic coordinates of at least a portion of PME-1 alone or in complex with PP2A;
determining spatial coordinates of the at least a portion of PME-1 alone or in complex with PP2A;
electronically screening stored spatial coordinates of candidate compounds against the spatial coordinates of the at least a portion of PME-1 alone or in complex with PP2A;
identifying a compound that is substantially similar to the at least a portion of PME-1 alone or in complex with PP2A; and
synthesizing the identified compound.

16. The method of claim 15, further comprising identifying a candidate compound that deviates from the atomic coordinates of the at least a portion of PME-1 alone or in complex with PP2A by a root mean square deviation of less than about 10 angstroms.

17. The method of claim 15, further comprising testing the identified compound for binding at least a portion of PME-1 alone or in complex with PP2A.

18. The method of claim 15, further comprising testing the identified compound for inhibiting PME-1 and/or PP2A activity.

19. The method of claim 15, further comprising testing the identified compound to determine if it inhibits or enhances methylation, tyrosine phosphorylation, serine phosphorylation, threonine phosphorylation or a combination thereof modulated by PME-1 and/or PP2A.

20. The method of claim 15, wherein the step of electronically screening stored spatial coordinates further comprises identifying a compound that has a shape, a charge distribution, a size or a combination thereof substantially similar to a portion of PME-1 alone or in complex with PP2A.

21. The method of claim 15, wherein the at least a portion of PME-1 alone or in complex with PP2A comprises the interface between PME-1 and the catalytic subunit of PP2A.

22. The method of claim 15 wherein the identified compound interrupts the interface between PME-1 and PP2A.

23. The method of claim 15, wherein the identified compound binds to PME-1.

24. A pharmaceutical composition comprising:
an effective amount of a compound having a three-dimensional structure corresponding to atomic coordinates of at least a portion of PME-1; and
a pharmaceutically acceptable excipient or carrier.

25. The pharmaceutical composition of claim 24, wherein the compound further comprises a three-dimensional structure corresponding to atomic coordinates of at least a portion of PME-1 in complex with PP2A.

26. The pharmaceutical composition of claim 24, wherein the compound binds to PME-1.

27. A system for identifying PME-1 and/or PP2A modulators comprising:
a processor; and
a processor readable storage medium in communication with the processor readable storage medium comprising the atomic coordinates of at least a portion of PME-1 alone or in complex with PP2A.

28. The system of claim 27, wherein the processor readable storage medium further comprises one or more programming instructions for:
applying a three-dimensional modeling algorithm to the atomic coordinates of PME-1 alone or in complex with PP2A;
determining spatial coordinates of at least a portion of PME-1 alone or in complex with PP2A;
electronically screening spatial coordinates of candidate compounds with the spatial coordinates of the at least a portion of PME-1 alone or in complex with PP2A; and
identifying a candidate compound whose spatial coordinates are substantially similar to the spatial coordinates of the at least a portion of PME-1 alone or in complex with PP2A; or
identifying a candidate compound whose spatial coordinates are substantially complementary to the spatial coordinates of the at least a portion of PME-1 alone or in complex with PP2A.

29. The system of claim 28, wherein the one or more programming instructions for identifying a candidate compound whose spatial coordinates are substantially similar to the spatial coordinates of the at least a portion of PME-1 alone or in complex with PP2A comprise one or more programming instructions for identifying a compound that deviates from the spatial coordinates of the at least a portion of PME-1 alone or in complex with PP2A by a user defined threshold.

30. The system of claim 28, wherein the one or more programming instructions for identifying a compound whose spatial coordinates are substantially similar to the at least a portion of PME-1 alone or in complex with PP2A comprise one or more programming instructions for identifying a compound having one or more of:
a size within a user defined threshold;
a charge within a user defined threshold; or
a shape with a user defined threshold.

31. The system of claim 28, wherein the one or more programming instructions for electronically screening spatial coordinates of a candidate compound comprises one or more programming instructions for simulating binding of the candidate compound to PME-1 alone or in complex with PP2A.

32. The system of claim 27, further comprising an output device in communication with the processor.

33. The system of claim 27, wherein the processor readable storage medium further comprises one or more programming instructions for:
applying a three-dimensional modeling algorithm to the atomic coordinates of PP2A holoenzyme;
determining spatial coordinates of at least a portion of PME-1 alone or in complex with PP2A;
generating a visual signal and relaying the visual signal to the output device; and
electronically designing a compound that is substantially similar to the at least a portion of PME-1 alone or in complex with PP2A; or
electronically designing a compound that is substantially complementary to the at least a portion of PME-1 alone or in complex with PP2A.

34. A PME-1 and/or PP2A binding compound comprising a molecule having a three-dimensional structure corresponding to atomic coordinates derived from at least a portion of an atomic model of PME-1 alone or in complex with PP2A.

35. The compound of claim 34, wherein the molecule is an inhibitor of PME-1 or PP2A.

36. The compound of claim 34, wherein the molecule has a three-dimensional structure corresponding to atomic coordinates of at least a portion subunit C of PP2A bound to PME-1,
wherein the compound makes interactions with the catalytic (C) subunit of protein phosphatase 2A (PP2A) holoenzyme that correspond to at least a portion of the interactions observed between the catalytic (C) subunit of protein phosphatase 2A (PP2A) holoenzyme and PME-1.

37. The compound of claim 36, wherein the molecule binds protein phosphatase 2A (PP2A) at a binding site for PME-1.

38. The compound of claim 34, wherein the molecule is substantially complementary to a portion of PME-1.

39. The compound of claim 34, wherein the molecule is substantially complementary to a portion of the catalytic (C) subunit of protein phosphatase 2A (PP2A) holoenzyme.

40. The compound of claim 34, wherein the molecule inhibits access of substrate to the active site of PME1.

41. The compound of claim 36, wherein the molecule binds to at least a portion of PME-1 with a greater affinity than a naturally occurring substrate.

42. The compound of claim 34, wherein the molecule inhibits PME-1 catalyzed activity, protein phosphatase 2A (PP2A) catalyzed activity, or a combination thereof.

43. The compound of claim 34, further comprising a pharmaceutically acceptable excipient or carrier.

44. The compound of claim 34, wherein the molecule deviates from the atomic coordinates of the at least a portion of PME-1 alone or in complex with PP2A by a root mean square deviation of less than about 10 angstroms.

45. The compound of claim 34, wherein the molecule deviates from the atomic coordinates of the at least a portion of PME-1 alone or in complex with PP2A by a root mean square deviation of less than about 2 angstroms.


SEQUENCE LISTING (5 sequences)

SEQ ID NO: 1 - protein, 386 aa, homo sapien
MSALEKSMHLGRLPSRPPLPGSGGSQSGAKMRMGPGRKRDFSPVPWSQYFESMEDVEVENETGKDTFRVYKSGSEGPVLLLLHGGGHSALSWAVFTAAIISRVQCRIVALDLRSHGETKVKNPEDLSAETMAKDVGNVVEAMYGDLPPPIMLIGHSMGGAIAVHTASSNLVPSLLGLCMIDVVEGTAMDALNSMQNFLRGRPKTFKSLENAIEWSVKSGQIRNLESARVSMVGQVKQCEGITSPEGSKSIVEGIIEEEEEDEEGSESISKRKKEDDMETKKDHPYTWRIELAKTEKYWDGWFRGLSNLFLSCPIPKLLLLAGVDRLDKDLTIGQMQGKFQMQVLPQCGHAVHEDAPDKVAEAVATFLIRHRFAEPIGGFQCVFPGC

SEQ ID NO: 2 - protein, 309 aa, homo sapiens
MDEKVFTKELDQWIEQLNECKQLSESQVKSLCEKAKEILTKESNVQEVRCPVTVCGDVHGQFHDLMELFRIGGKSPDTNYLFMGDYVDRGYYSVETVTLLVALKVRYRERITILRGNHESRQITQVYGFYDECLRKYGNANVWKYFTDLFDYLPLTALVDGQIFCLHGGLSPSIDTLDHIRALDRLQEVPHEGPMCDLLWSDPDDRGGWGISPRGAGYTFGQDISETFNHANGLTLVSRAHQLVMEGYNWCHDRNVVTIFSAPNYCYRCGNQAAIMELDDTLKYSFLQFDPAPRRGEPHVTRRTPDYFL

SEQ ID NO: 3 - protein, 589 aa, homo sapiens
MAAADGDDSLYPIAVLIDELRNEDVQLRLNSIKKLSTIALALGVERTRSELLPFLTDTIYDEDEVLLALAEQLGTFTTLVGGPEYVHCLLPPLESLATVEETVVRDKAVESLRAISHEHSPSDLEAHFVPLVKRLAGGDWFTSRTSACGLFSVCYPRVSSAVKAELRQYFRNLCSDDTPMVRRAAASKLGEFAKVLELDNVKSEIIPMFSNLASDEQDSVRLLAVEACVNIAQLLPQEDLEALVMPTLRQAAEDKSWRVRYMVADKFTELQKAVGPEITKTDLVPAFQNLMKDCEAEVRAAASHKVKEFCENLSADCRENVIMSQILPCIKELVSDANQHVKSALASVIMGLSPILGKDNTIEHLLPLFLAQLKDECPEVRLNIISNLDCVNEVIGIRQLSQSLLPAIVELAEDAKWRVRLAIIEYMPLLAGQLGVEFFDEKLNSLCMAWLVDHVYAIREAATSNLKKLVEKFGKEWAHATIIPKVLAMSGDPNYLHRMTTLFCINVLSEVCGQDITTKHMLPTVLRMAGDPVANVRFNVAKSLQKIGPILDNSTLQSEVKPILEKLTQDQDVDVKYFAQEALTVLSLA

SEQ ID NO: 4 - protein, 436 aa, homo sapiens
MVVDAANSNGPFQPVVLLHIRDVPPADQEKLFIQKLRQCCVLFDFVSDPLSDLKWKEVKRAALSEMVEYITHNRNVITEPIYPEVVHMFAVNMFRTLPPSSNPTGAEFDPEEDEPTLEAAWPHLQLVYEFFLRFLESPDFQPNIAKKYIDQKFVLQLLELFDSEDPRERDFLKTTLHRIYGKFLGLRAYIRKQINNIFYRFIYETEHHNGIAELLEILGSIINGFALPLKEEHKIFLLKVLLPLHKVKSLSVYHPQLAYCVVQFLEKDSTLTEPVVMALLKYWPKTHSPKEVMFLNELEEILDVIEPSEFVKIMEPLFRQLAKCVSSPHFQVAERALYYWNNEYIMSLISDNAAKILPIMFPSLYRNSKTHWNKTIHGLIYNALKLFMEMNQKLFDDCTQQFKAEKLKEKLKMKEREEAWVKIENLAKANPQVLKK

SEQ ID NO: 5 - protein, 6 aa, homo sapiens
TPDYFL